SEQUENCE LISTING (2 sequences)

SEQ ID NO: 1 - DNA, 24 bp, Artificial Sequence
  Description of Artificial Sequence Synthetic probe
agcttcatttcccgtaaatcccta

SEQ ID NO: 2 - DNA, 18 bp, Artificial Sequence
  Description of Artificial Sequence Synthetic probe
agatttctaggaattcaa

(19) United States
(12) Patent Application Publication
(10) Pub. No.: US 2022/0289720 A1
(43) Pub. Date: Sep. 15, 2022

(54) ARYL SULFONAMIDES AS SMALL MOLECULE STAT3 INHIBITORS

(71) Applicant: UNIVERSITY OF HAWAII, Honolulu, HI (US)

(72) Inventors: James TURKSON, Honolulu, HI (US); Francisco LOPEZ-TAPIA, Honolulu, HI (US); Marcus TIUS, Honolulu, HI (US); Peibin YUE, Honolulu, HI (US); Christine BROTHERTON-PLEISS, Sunnyvale, CA (US); Wenzhen FU, Honolulu, HI (US)

(21) Appl. No.: 17/628,513

(22) PCT Filed: Jul. 22, 2020
(86) PCT No.: PCT/US2020/043037

§ 371 Date: Jan. 19, 2022

Related U.S. Application Data
(60) Provisional application No. 62/877,243, filed on Jul. 22, 2019.

(51) Int. Cl. C07D 403/12; C07D 401/14; C07D 401/12; C07D 205/04; C07D 213/81; C07D 403/14; C07C 317/36; C07D 237/32; C07D 405/14; A61P 35/00

(52) U.S. Cl. CPC C07D 403/12; C07D 401/14; C07D 401/12; C07D 205/04; C07D 213/81; C07D 403/14; C07C 317/36; C07D 237/32; C07D 405/14; A61P 35/00; C07C 2601/16

(57) ABSTRACT

The present disclosure provides pharmaceutical compositions comprising aryl sulfonamide Stat3 small molecule inhibitors and certain pharmaceutically acceptable salts thereof, and methods of their use for treating cancer.

### GOVERNMENT RIGHTS

This invention was made with government support under grant number R01 CA208851 awarded by the National Institutes of Health. The government has certain rights in the invention.

### FIELD

The present disclosure is generally related to potent aryl sulfonamide derivatized Stat3 small molecule inhibitors of Formulae I-V, and solvates, hydrates, and pharmaceutically acceptable salts thereof. The present disclosure also relates to pharmaceutical compositions containing the inhibitors and their use in the treatment or prevention of cancer, and other pathogenic conditions in which Stat3 activation is implicated. As an example, the disclosure provides methods and compositions for the treatment of cancer by modulating Stat3.

### CROSS REFERENCE TO THE RELATED APPLICATIONS

The present application claims the benefit of U.S. Provisional Patent Application Ser. No. 62/877,243, filed on Jul. 22, 2019, which is incorporated herein by reference in its entirety for all purposes.

### BACKGROUND

The following includes information that may be useful in understanding various aspects and embodiments of the present disclosure. It is not an admission that any of the information provided herein is prior art, or relevant, to the presently described or claimed inventions, or that any publication or document that is specifically or implicitly referenced is prior art.

The signal transducer and activator of transcription (Stat) family of cytoplasmic transcription factors have important roles in many cellular processes, including cell growth and differentiation, inflammation and immune responses. (Bromberg, et al., Breast Cancer Res. 2:86-90 (2000); Darnell, J., et al., Nat. Rev. Cancer 2:740-749 (2002)). STAT proteins are classically activated by tyrosine (Tyr) kinases, such as Janus kinases (JAKs) and Src family kinases, in response to the binding of cytokine and growth factors to their cognate receptors. The Tyr phosphorylation (pTyr) promotes dimerization between two activated STAT:STAT monomers through a reciprocal pTyr-Src homology SH2 domain interactions. Active STAT:STAT dimers translocate to the nucleus to induce gene transcription by binding to specific DNA-response elements in the promoters of target genes to regulate gene expression. By contrast, aberrantly-active Stat3, one of the Stat family members, has been implicated in many human tumors and represents an attractive target for drug discovery. The aberrant activation of Stat3 occurs in glioma, breast, prostate, ovarian, and many other human cancers, whereby it promotes malignant progression (Yu & Jove, Nat. Rev. Cancer 4:97-105 (2004)). Mechanisms by which constitutively-active Stat3 mediates tumorigenesis include dysregulation of gene expression that leads to uncontrolled growth and survival of tumor cells, enhanced tumor angiogenesis, and metastasis and the suppression of tumor immune surveillance (Yu & Jove (2004); Bromberg & Darnell, Oncogene 19:2468-2473 (2000); Bowman et al., Oncogene 19:2474-2488 (2000); Turkson J & Jove, Oncogene 19:6613-6626 (2000); Turkson, Expert Opin Ther Targets 8:409-422 (2004); Wang et al., Nat Med 10:48-54 (2004)).

Stat3 modulates mitochondrial functions and Stat3 crosstalk with other proteins, such as NF-κB, that promotes the malignant phenotype. Many human tumors harbor aberrantly-active signal transducer and activator of transcription Stat3 signaling.

### SUMMARY OF THE INVENTION

In one aspect, this invention relates to potent aryl sulfonamide derivatized Stat3 inhibitors, useful as cancer therapeutics. In some aspects, the compounds of this invention are useful for inhibiting malignant transformation, tumor development and progression.

In one aspect, this invention relates to compounds of Formula I, which selectively inhibit Stat3.

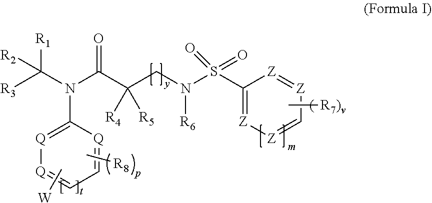

wherein R¹ is selected from aryl or a 5 or 6-membered aryl or heteroaryl, where the heteroatoms are one or more O, N, S(A)₂, where S is sulfur and A is selected from oxygen or an electron pair, the aryl or the 5 or 6-membered heteroaryl are optionally substituted with halogen, CF₃, C₁-C₆ alkyl, C₁-C₆ branched alkyl, aryl (which is optionally further substituted with 1-5 halogens), heteroaryl, C₃-C₇ cycloalkyl, C₃-C₇ cycloalkenyl, three- to six-membered heterocycle, three- to seven-membered saturated heterocycle, fused C₂-C₅ alkylene, where one or more CH₂ groups can be replaced with O, NR⁹, S(A)₂ where S is sulfur and A is selected from oxygen or an electron pair, the aryl or the 5 or 6-membered heteroaryl are optionally substituted naphthalene, optionally substituted indole, benzofuran, benzothiophene, benzimidazole; R² and R³ are independently selected from H or C₁-C₆ alkyl; where R² and R³ together can form a C₃-C₆ cycloalkane ring, where said C₃-C₆ cycloalkane ring can be substituted with 1 or more of C₁-C₆ alkyl, hydroxyl, NR⁹R¹⁰, or C₁-C₆ alkoxy; R⁴ is selected from H, C₁-C₆ alkyl, (CH₂)_{f}NR⁹R¹⁰, (CH₂)_{f}OR⁹, (CH₂)_{f}CO₂R⁹, (CH₂)_{f}CO₂NR⁹R¹⁰; R⁵ is selected from H, C₁-C₆ alkyl, (CH₂)_{f}NR⁹R¹⁰, (CH₂)_{f}OR⁹, (CH₂)_{f}CO₂R⁹, (CH₂)_{f}CO₂NR⁹R¹⁰, where R⁴ and R⁵ together can form a C₃-C₆ cycloalkane ring, where said C₃-C₆ cycloalkane ring can be substituted with 1 or more of C₁-C₆ alkyl, hydroxyl, NR⁹R¹⁰, or C₁-C₆ alkoxy, where one or more CH₂ groups can be replaced with O, NR⁹, S(A)₂ where S is sulfur and A is selected from oxygen or an electron pair; R⁶ is selected from C₁-C₆ alkyl; where R⁴ and R⁶ together can form a C₃-C₆ N-heterocycle ring, where said C₃-C₆ N-heterocycle ring can be substituted with 1 or more of C₁-C₆ alkyl, hydroxyl, NR⁹R¹⁰ and where one or more CH₂ groups of said C₁-C₆ alkyl can be replaced with O, NR⁹, S(A)₂ where S is sulfur and A is selected from oxygen or an electron pair; wherein R⁴ and R⁶ together can form an optionally substituted pyrrole ring, wherein one or more CH groups of said pyrrole ring can be replaced with O, N, S(A)₂, where S is sulfur and A is selected from oxygen or an electron pair; where N of said pyrrole ring can be replaced with C and R⁴ and R⁶ together can form aryl, heteroaryl, C₃-C₇ cycloalkyl or C₃-C₇ cycloalkenyl ring, where said aryl, heteroaryl, C₃-C₇ cycloalkyl or C₃-C₇ cycloalkenyl ring can be substituted with 1 or more of C₁-C₆ alkyl, hydroxyl, NR⁹R¹⁰ and where one or more CH₂ groups can be replaced with O, NR⁹, S(A)₂ where S is sulfur and A is selected from oxygen or an electron pair; each R⁷ is independently selected from C₁-C₆ alkyl, halogen, hydroxyl, CN, CF₃, C₁-C₆ alkyl- or dialkyl-amino, C₁-C₆ branched alkyl- or dialkylamino, or C₁-C₆ alkyl- or C₁-C₆ branched alkyl ether; each R⁸ is a substitution independently selected from one or more of H, C₁-C₆ alkyl, C₁-C₆ alkoxy, hydroxyl, halogen, OC(O)CH₃, NR⁹R¹⁰, CN, CF₃, CO₂R⁹, CO₂NR⁹R¹⁰, (CH₂)_{f}NR⁹R¹⁰, (CH₂)_{f}OR⁹, or (CH₂)_{f}CO₂R⁹; R⁹ is selected from H or C₁-C₆ alkyl; R¹⁰ is selected from H, C₁-C₆ alkyl; W is selected from H, CO₂H, tetrazole, benzyl, C(O)NHOR¹⁰ and CF₂OH; each instance of Q is independently selected from C, CH, N, O, or S; or where Q and R⁸ together can form a 5 or 6 membered lactone or lactam ring, or a heterocyclic ring; each instance of Z is independently selected from C, CH, N, O, or S; p is selected from 0 or 1, y is selected from 0 or 1, f is selected from 0 to 4; t is selected from 0 or 1, v is an integer selected from 1 to 5, m is selected from 0 or 1, and solvates, hydrates, or pharmaceutically acceptable salts thereof.

In one aspect, this invention relates to compounds of Formula II:

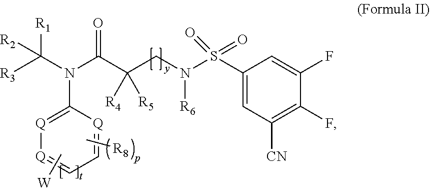

and prodrugs, solvates, hydrates, or pharmaceutically acceptable salts thereof.

In one aspect, this invention relates to compounds of Formula III:

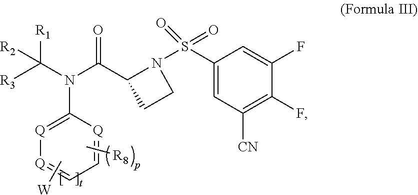

and prodrugs, solvates, hydrates, or pharmaceutically acceptable salts thereof. The compounds of Formula III have a core aryl sulfonamido azetidine structure.

In one aspect, this invention relates to compounds of Formula IV:

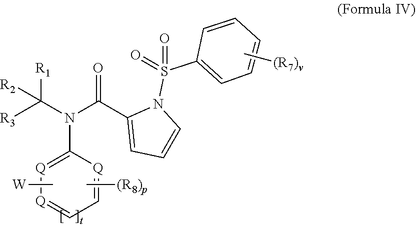

and prodrugs, solvates, hydrates, or pharmaceutically acceptable salts thereof.

In one aspect, this invention relates to compounds of Formula V:

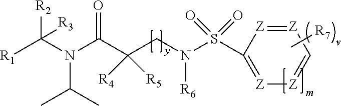

and prodrugs, solvates, hydrates, or pharmaceutically acceptable salts thereof.

In one aspect, this invention relates to compounds of Formula I where R⁵ is H.

In one aspect, this invention relates to compounds of Formula I where R⁴ and R⁶ together form a C₃-C₆ N-heterocycle ring, where said C₃-C₆ N-heterocycle ring can be substituted with 1 or more of C₁-C₆ alkyl, hydroxyl, NR⁹R¹⁰ and where one or more CH₂ groups of said C₁-C₆ alkyl can be replaced with O, NR⁹, S(A)₂ where S is sulfur and A is selected from oxygen or an electron pair; or where R⁴ and R⁶ form an optionally substituted pyrrole ring, wherein one or more CH groups of said pyrrole ring can be replaced with O, N, S(A)₂, where S is sulfur and A is selected from oxygen or an electron pair; where N of said pyrrole ring can be replaced with C and R⁴ and R⁶ can form aryl, heteroaryl, C₃-C₇ cycloalkyl or C₃-C₇ cycloalkenyl ring, where said aryl, heteroaryl, C₃-C₇ cycloalkyl or C₃-C₇ cycloalkenyl ring can be substituted with 1 or more of C₁-C₆ alkyl, hydroxyl, NR⁹R¹⁰ and where one or more CH₂ groups can be replaced with O, NR⁹, S(A)₂ where S is sulfur and A is selected from oxygen or an electron pair.

In one aspect, this invention relates to compounds of Formula I where R⁷ is independently selected from aryl or heteroaryl group where the aryl or heteroaryl group is substituted with 1-5 substituents independently selected from C₁-C₆ alkyl- or dialkyl-amino, C₁-C₆ branched alkyl- or dialkylamino, or C₁-C₆ alkyl- or C₁-C₆ branched alkyl ether, or halogen, or CN.

In one aspect, this invention relates to compounds of Formula I where W is H.

In one aspect, this invention relates to a compound having one of the following formulae:

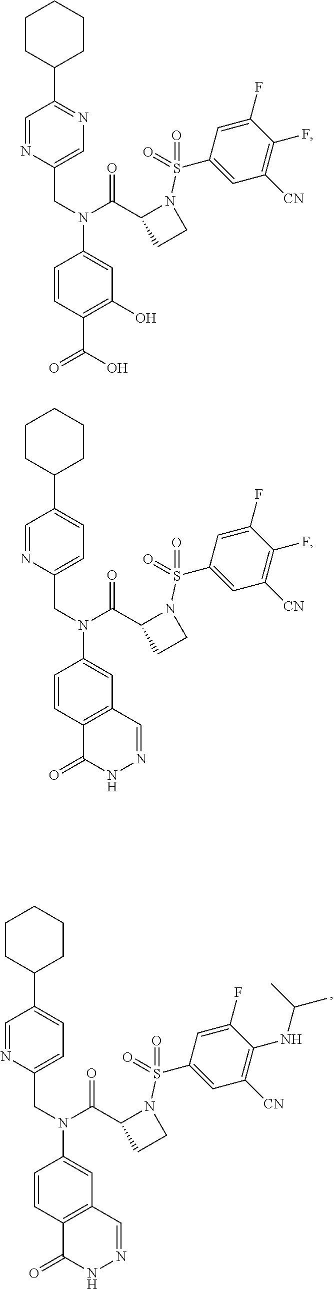

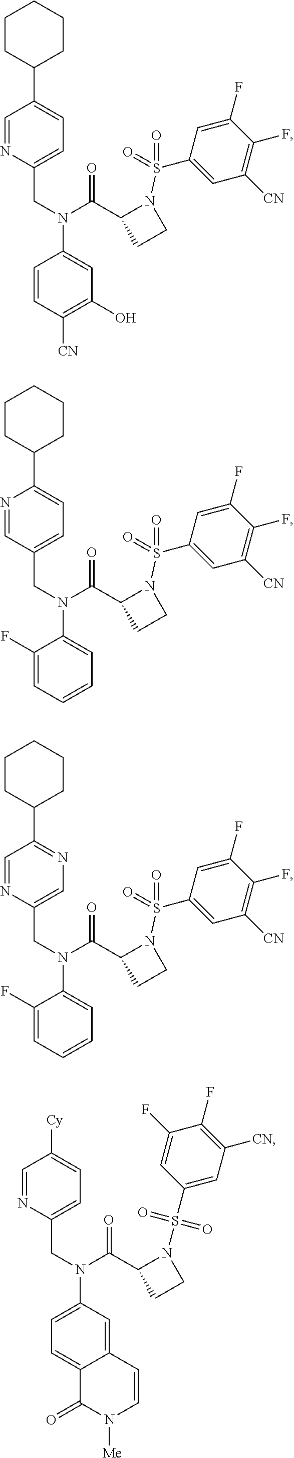

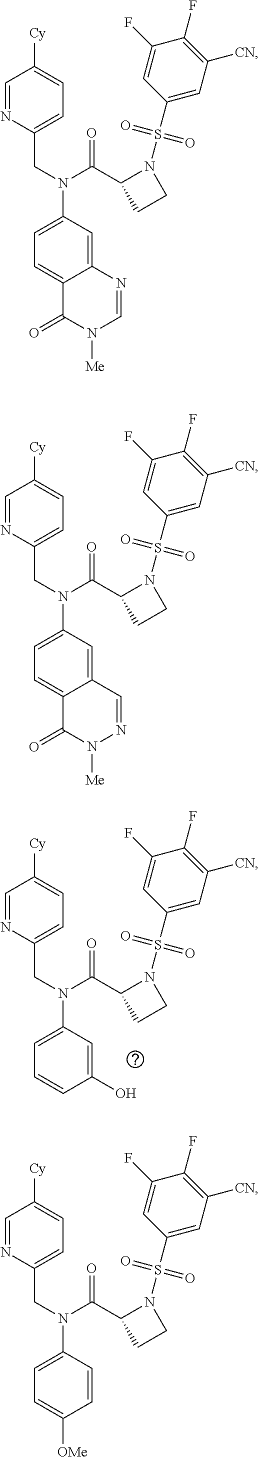

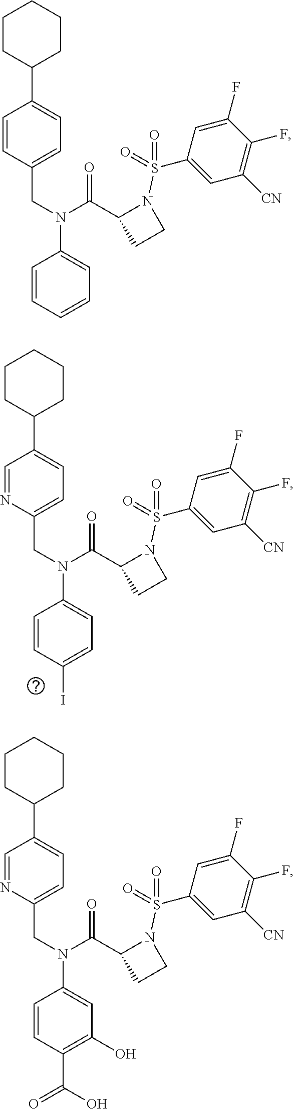

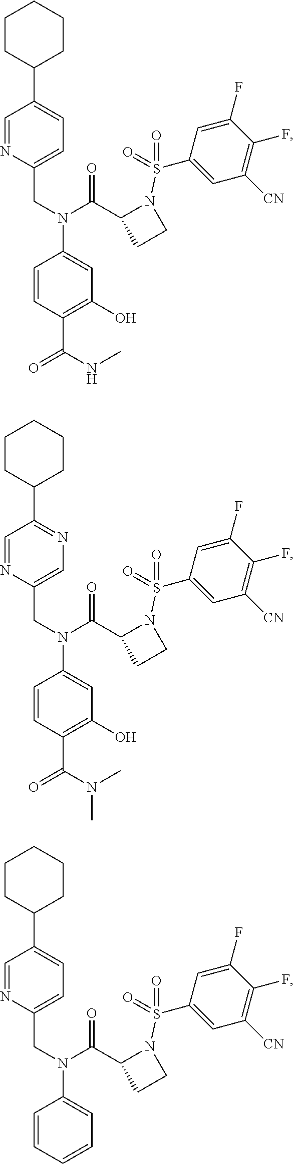

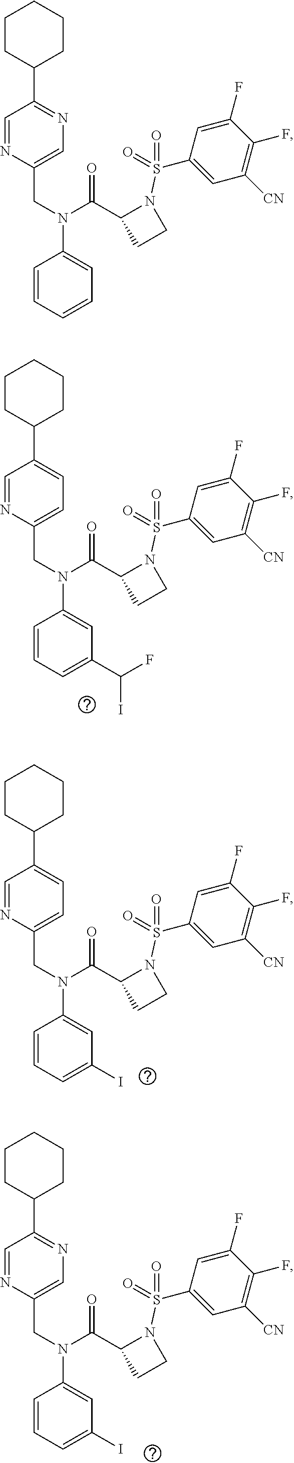

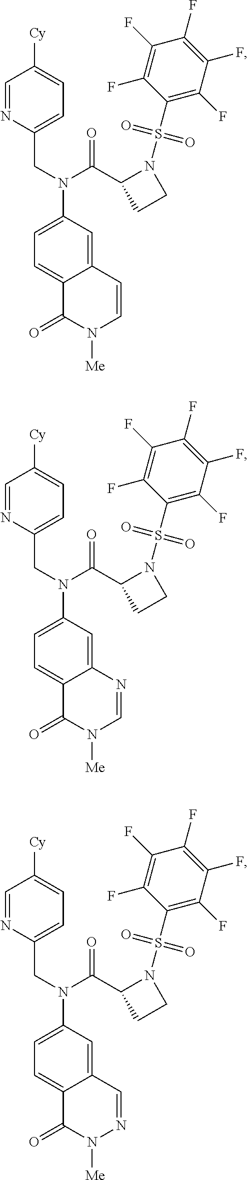

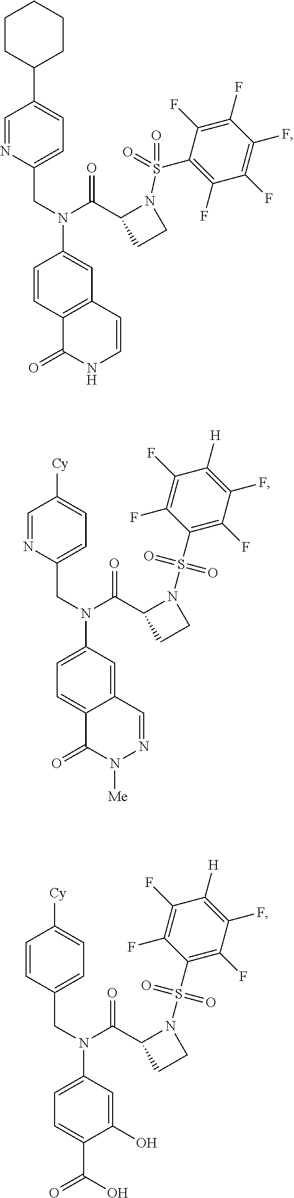

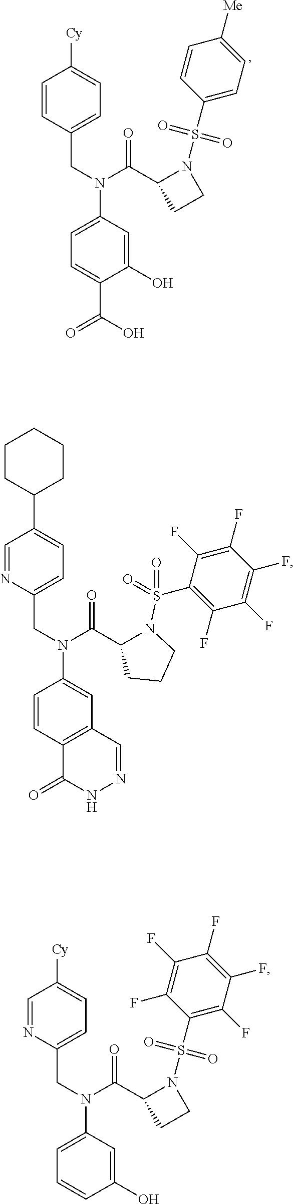

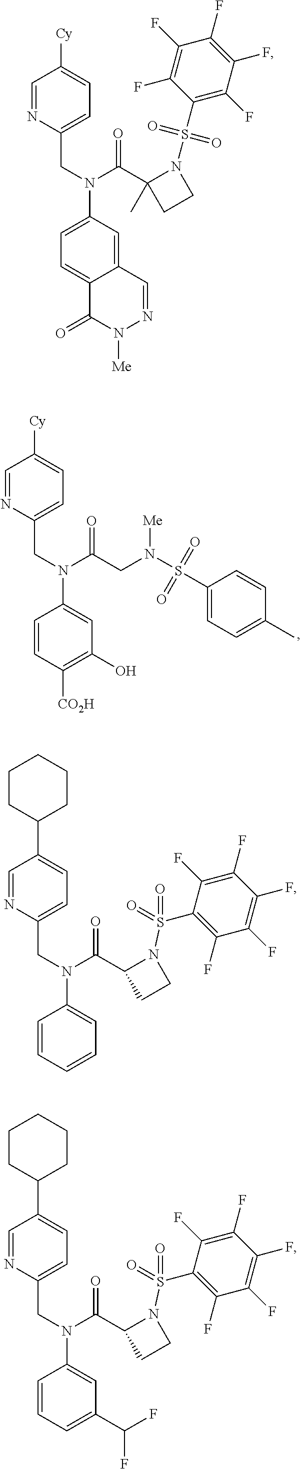

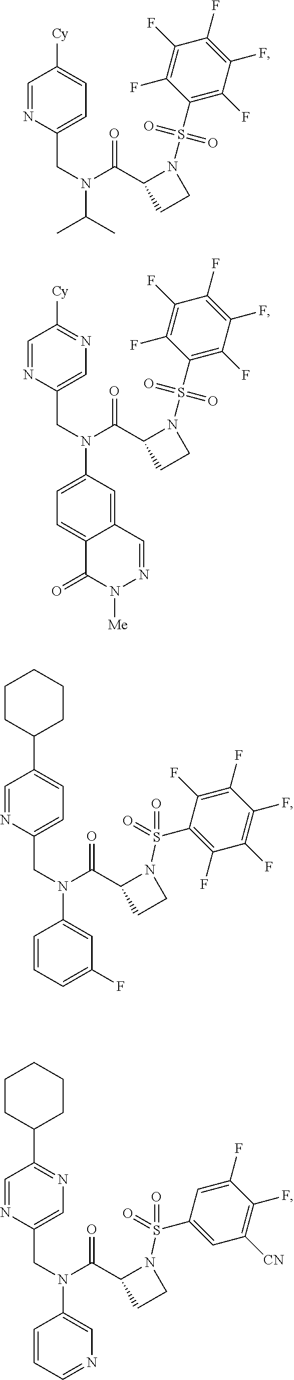

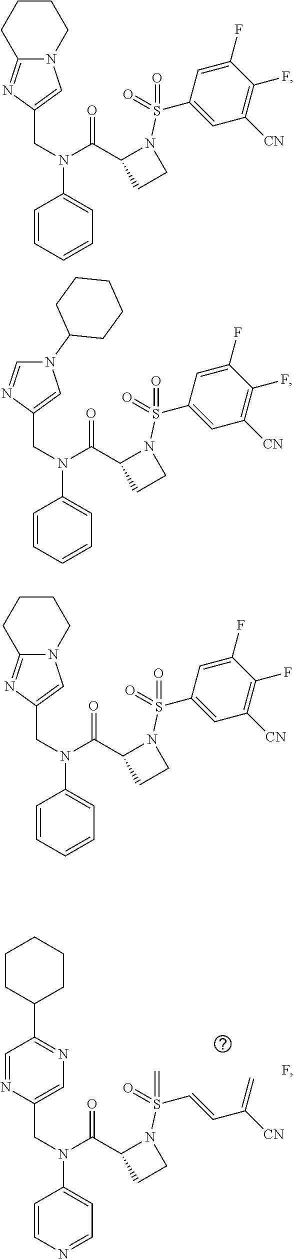

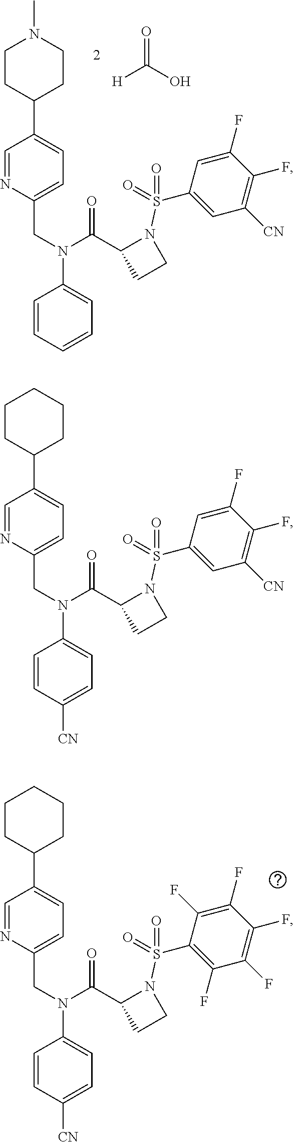

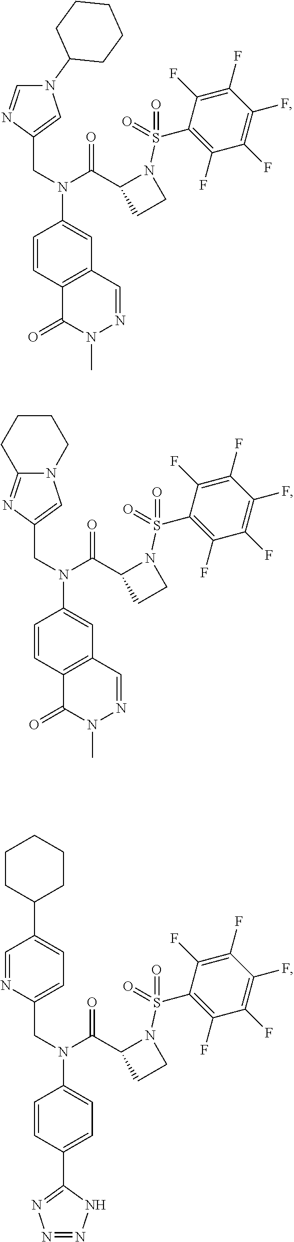

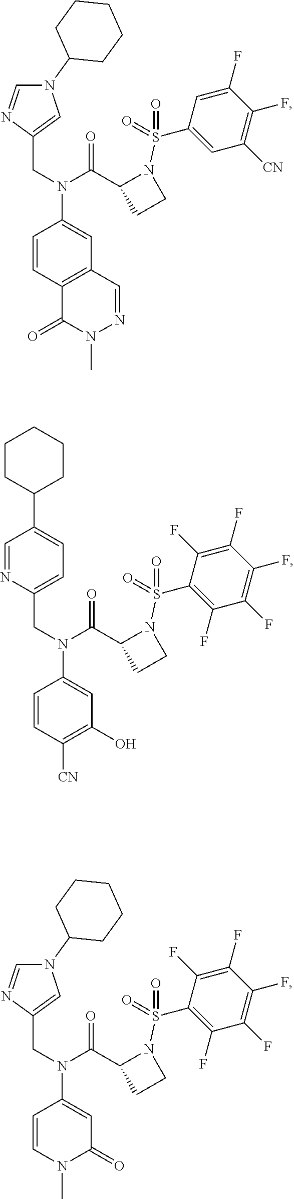

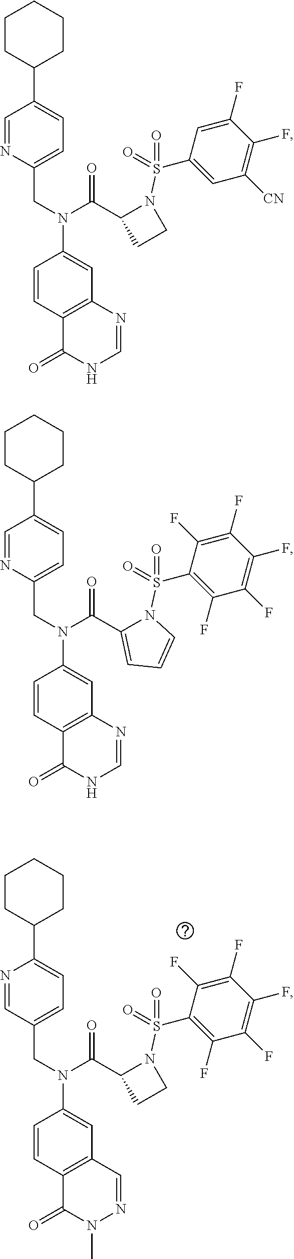

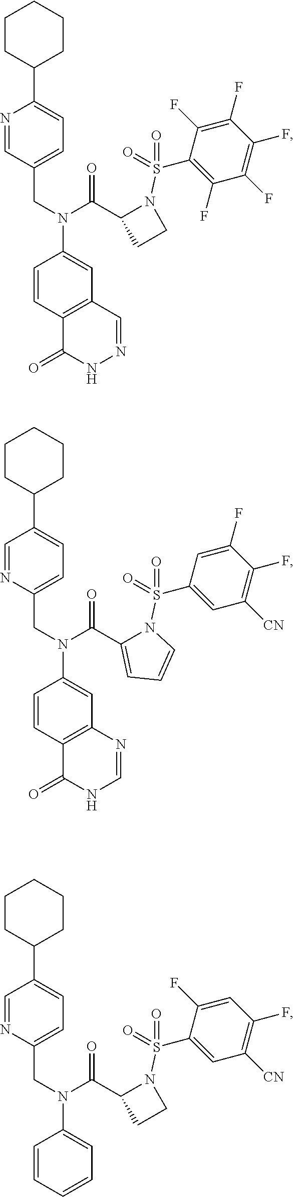

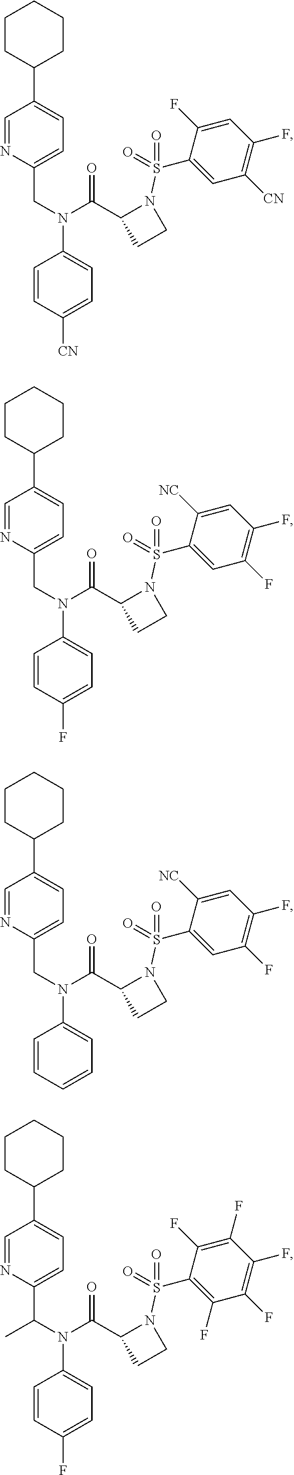

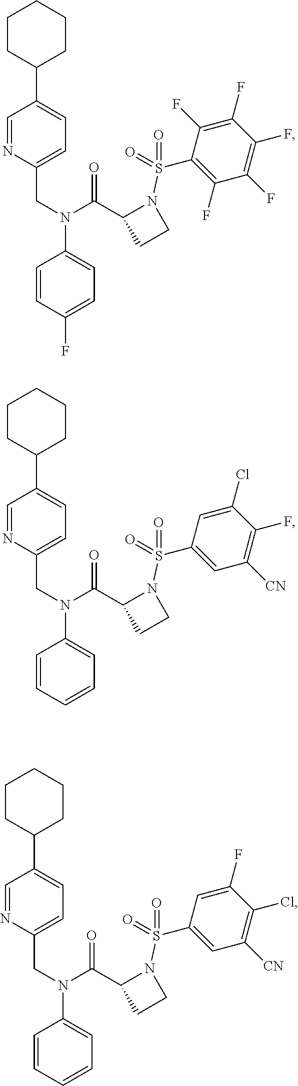

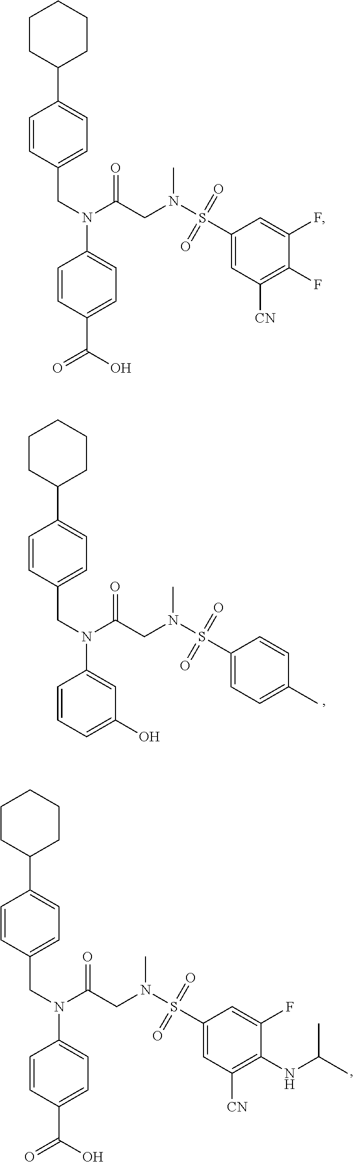

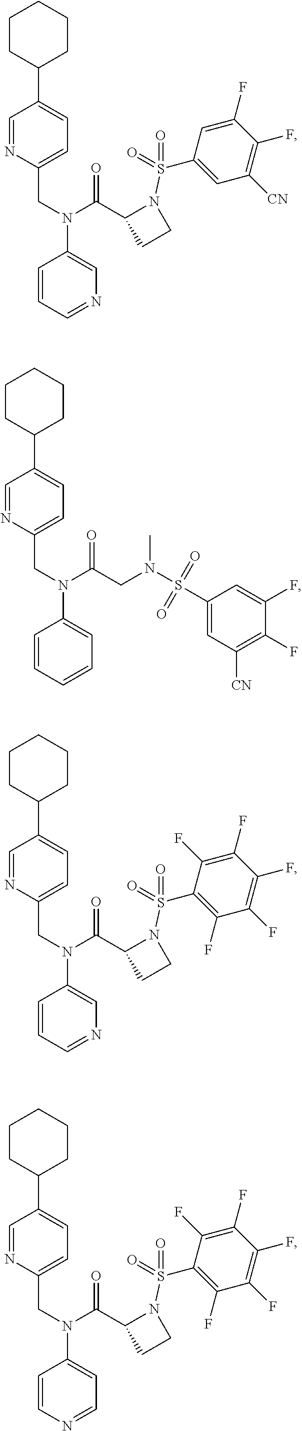

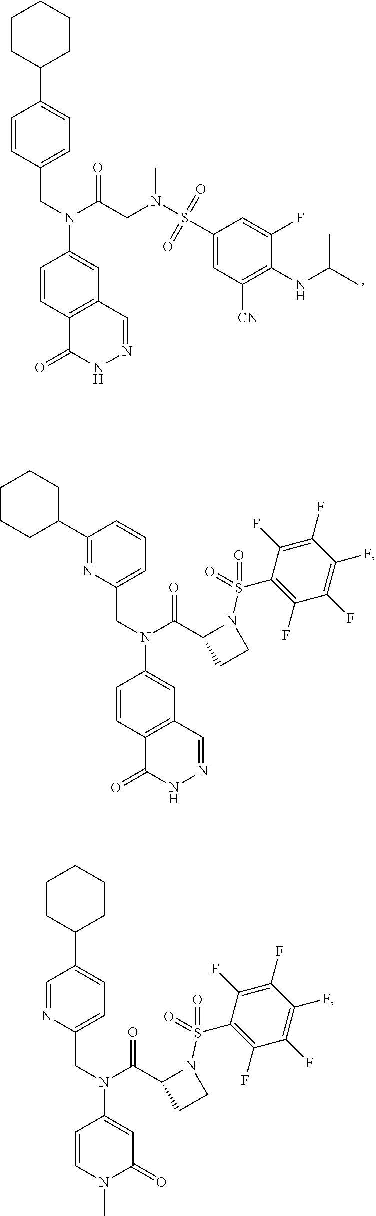

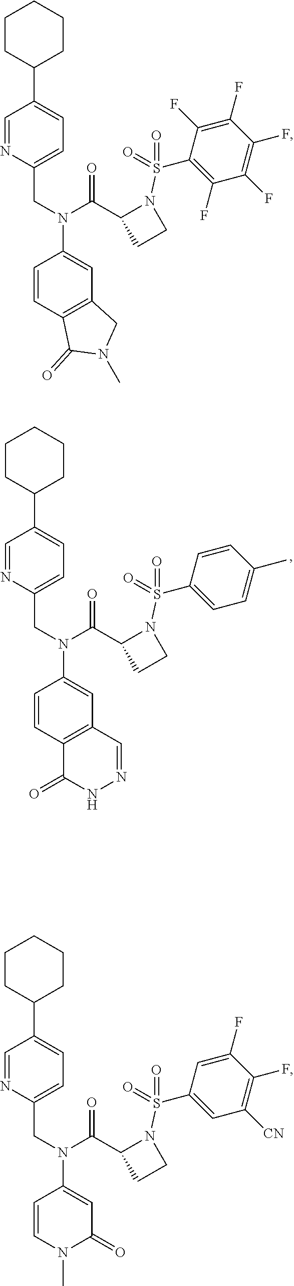

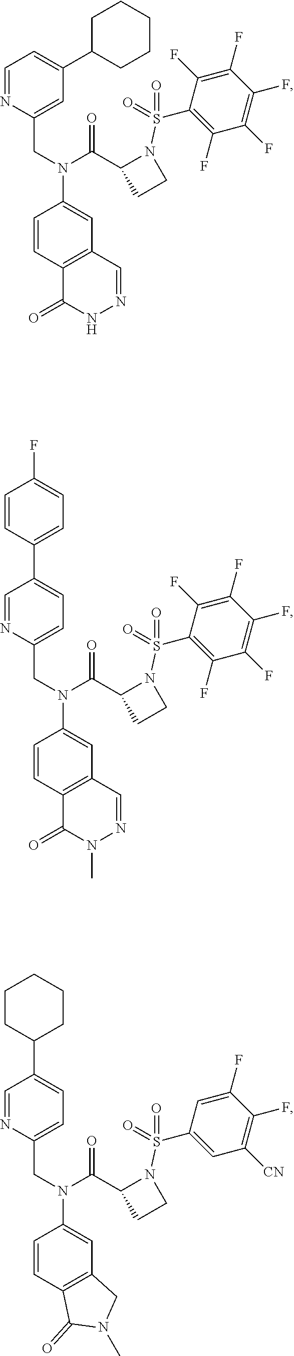

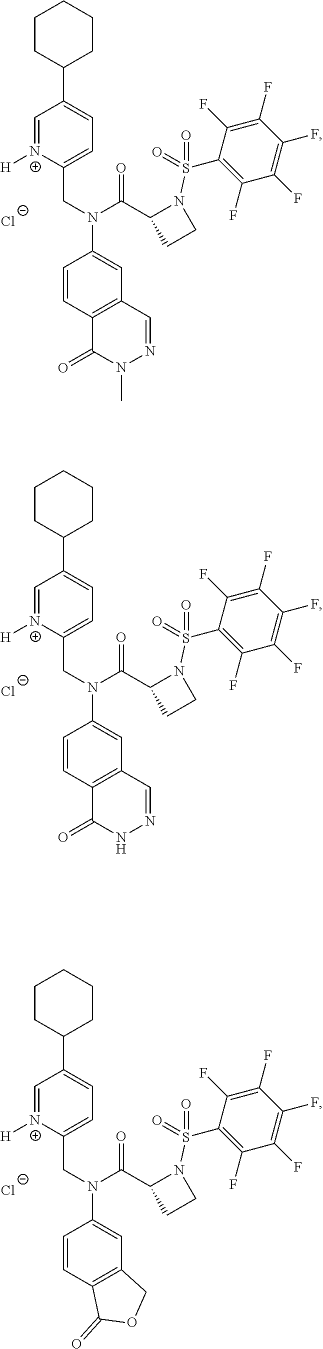

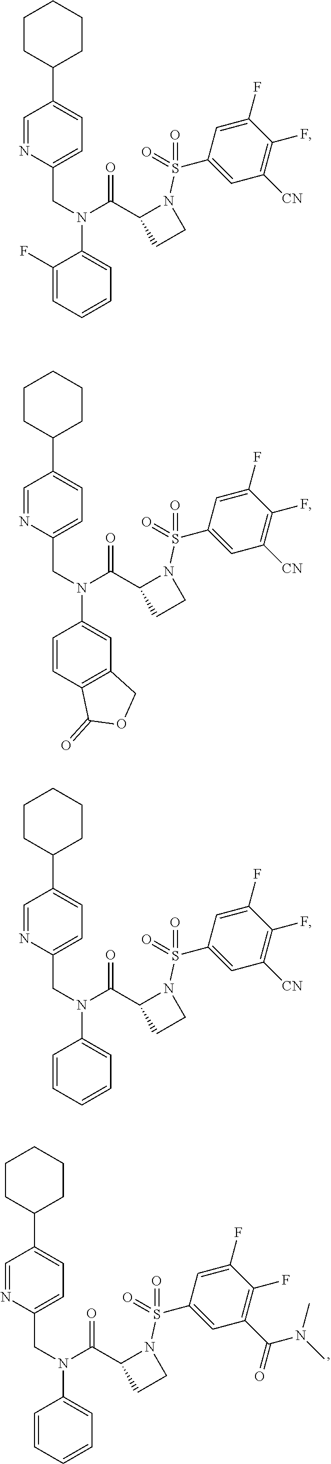

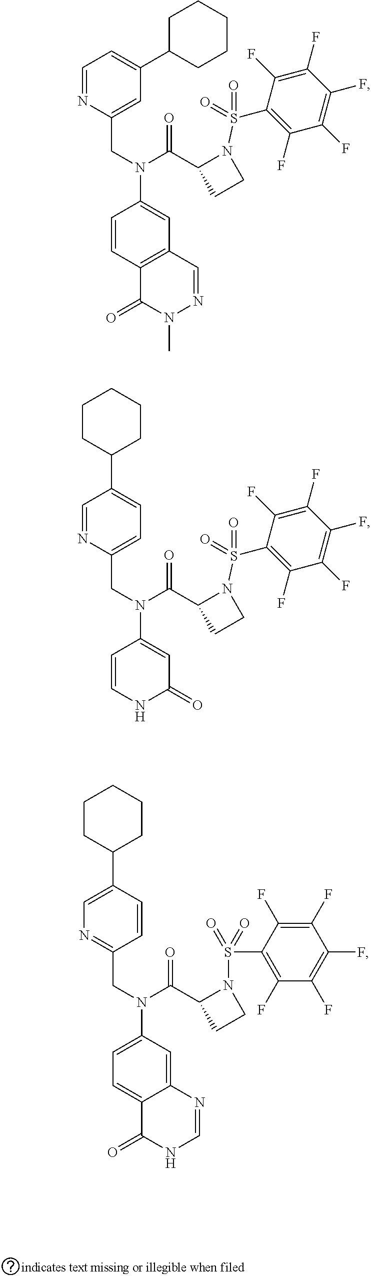

wherein Cy is cyclopropyl, and prodrugs, solvates, hydrates, or pharmaceutically acceptable salts thereof.

In one aspect, this invention relates to one of the following compounds:

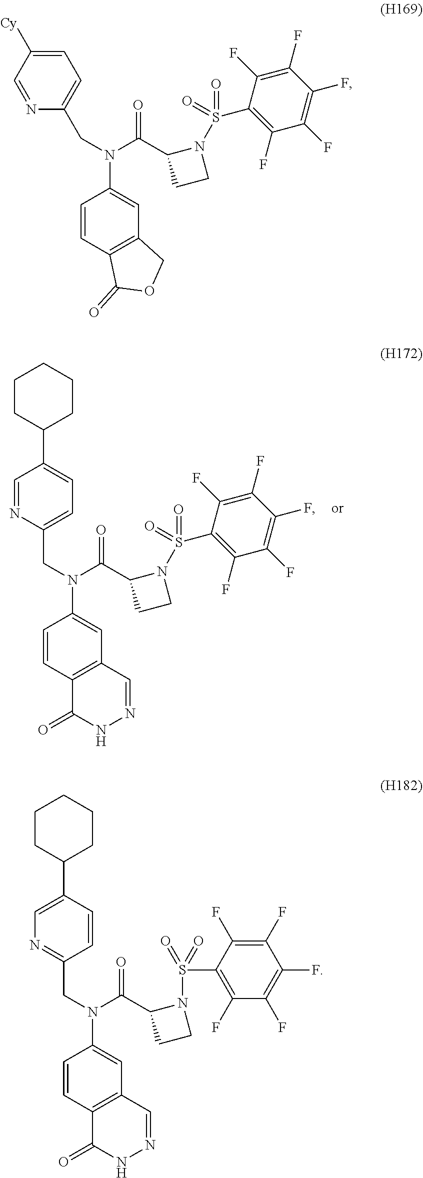

In one aspect, this invention relates to a composition comprising a therapeutically effective amount of a compound, solvate, hydrate, or pharmaceutically acceptable salt, of any compound of Formula I-V, including those of Examples 1-88, and a pharmaceutically acceptable excipient.

In one aspect, this invention relates to a composition for use in selectively treating tumor cells having a constituitively activated Stat3, comprising a therapeutically effective amount of any compound of Formula I-V, including those of Examples 1-88.

In one aspect, this invention relates to a compound of Formula I-V, including those of Examples 1-88 having a Stat3 DNA-binding activity as measured by electrophoretic mobility shift assay (EMSA) of less than 5 micromolar, preferably of less than 1 micromolar.

In one aspect, this invention relates to a method of administering a composition comprising a therapeutically effective amount of any compound of Formula I-V, including those of Examples 1-88 to a subject, wherein survival, growth or migration of a cell harboring abberantly active Stat3 is inhibited.

In one aspect, this invention relates to a method of treating cancer, comprising administering to a subject in need thereof, a therapeutically effective amount of a composition comprising a therapeutically effective amount of any compound of Formula I-V, including those of Examples 1-88. In some aspects, the effective dose of the composition ranges from about 0.05 mg/kg to about 5 g/kg, from about 0.08 mg/kg to about 0.5 mg/kg, from about 0.08 to about 0.24 mg/kg, or from about 0.24 to about 0.5 mg/kg, or from about 0.08 to 0.5 mg/kg. In some aspects, the one or more effective doses of the composition are administered orally, subcutaneously, intravenously, or intramuscularly. In some aspects, the cancer is a solid tumor, preferably a solid tumor which is selected from glioma, breast cancer, pancreatic cancer, lung cancer, prostate cancer, ovarian cancer, bladder cancer, head and neck cancer, thyroid cancer, brain cancer, skin cancer and kidney cancer. In some aspects, the cancer is selected from the group consisting of: medulloblastomas, cerebral menangiomas, malignant melanoma, multiple myeloma, lymphomas, including anaplastic large T cell lymphoma, sezary syndrome, EBV-related Burkitt's Lymphoma, HSV Saimiri-dependent (T Cell), cutaneous T cell lymphoma, mycosis fungoides, leukemia, including HTLV-I dependent leukemia, erythroleukemia, acute lymphocytic leukemia (ALL), chronic lymphocytic leukemia (CLL), acute myelogenous leukemia (AML), chronic myelogenous leukemia (CML), megakaryocytic leukemia, and large granula lymphocyte (LGL) leukemia, renal cell carcinoma, pancreatic adenocarcinoma, ovarian carcinoma, squamous cell carcinoma of the head and neck, and Hodgkin's Lymphoma.

In some aspects, this invention relates to the use of a compound of any compound of Formula I-V, including those of Examples 1-88 for the preparation of a medicament for the treatment of a condition selected from the group consisting of cancer, hyperplasia, or neoplasia.

In some aspects, any R group described herein can include or exclude the recited options.

In one aspect, the compounds of this invention inhibit Stat3. The compounds of this invention uniquely interact with three sub-pockets on the stat3:stat3 dimer interface, in contrast to other previously described Stat3 inhibitors, which interacts with only two sub-pockets. As a result of the unique and specific mechanism by which the aryl sulfonamide derivatized Stat3 inhibitors of this invention exert their effects, the compounds are more potent and less toxic. The compounds of this invention also surprisingly selectively bind the activated form of Stat3, consequently attenuating Stat3 functions in cancer cells. The compounds of this invention are useful, for example, for inhibiting cancer cell growth, survival, migration and/or metastasis.

In one aspect, this invention relates to compounds which preferentially inhibit Stat3 DNA-binding activity with IC₅₀'s of 10 μM or less. In one aspect, this invention relates to compounds which preferentially inhibit Stat3 DNA-binding activity with IC₅₀'s of 5 μM or less, preferably 1 μM or less, as measured by EMSA analysis. In one aspect, this invention relates to compositions and formulations useful for inhibiting cancer growth. In some aspects, the anti-cancer activity of the compounds is determined by the ability to inhibit growth of mouse xenografts of human breast and non-small cell lung cancers.

Dimerization of Stat3 occurs through SH2-phosphotyrosyl peptide interactions. See Shuai et al., Interferon activation of the transcription factor Stat91 involves dimerization through SH2-phosphotyrosyl peptide interactions (Cell, 76:821-828 (1994); Miklossy et al. Nat Rev Drug Discov 12:611-629 (2013); Turkson et al., Mol Cancer Ther 3:261-269 (2004); Turkson et al., J. Biol. Chem. 276:45443-45455 (2001); Siddiquee et al., Proc Natl Acad Sci USA. 104:7391-7396 (2007); Coleman et al., J Med Chem. 48(6661-70) (2005)).

In one aspect, the invention relates to the inventors' design of aryl sulfonamide derivatized Stat3 inhibitors which interfere with the dimerization between two monomers, and the inventors' recognition that this represents an attractive strategy to develop drugs that inhibit Stat3 activation and functions.

The present disclosure provides novel, selective aryl sulfonamide derivatized Stat3 inhibitors, and pharmaceutical formulations and kits comprising the inhibitors. The compounds and pharmaceutical formulations are useful as therapeutics for cancer and other conditions mediated by aberrantly active Stat3, a substrate for growth factor receptor tyrosine kinases, or cytoplasmic tyrosine kinases, including Janus kinases or the Src family kinases. In some aspects, the processes inhibited by the compounds and compositions of this invention include proliferation, survival, angiogenesis, migration/metastasis/invasion, and immunity.

The compounds of this invention are useful for inhibiting activities resulting from constitutive Stat3 activation, which include: a) stimulating proliferation by increasing the expression of c-Myc and/or cyclin D1/D2, and/or decreasing expression of p53; b) increasing survival by increasing the expression of survivin, Bcl-x/Bcl-2, Mc1-1 and/or Akt-2; stimulating angiogenesis by increasing expression of VEGF; and/or increasing migration/metastasis or invasion by increasing the expression MMP-2 or MMP-9.

In one aspect, the present disclosure provides the use of a compound of any of Formulae I-V including compounds of Examples 1-88 for the preparation of a medicament for the treatment of a condition selected from the group consisting of cancer, hyperplasia, autoimmune indications, and neoplasia. In one aspect, the tumor progression, including metastasis and/or growth is thereby inhibited and/or reduced. In one aspect, multi-drug resistance is thereby inhibited and/or reduced.

In another aspect the present disclosure provides a method of treating cancer comprising administering to a subject in need thereof a therapeutically effective amount of a pharmaceutical composition comprising a compound of any of Formulae I-V including compounds of Examples 1-88 whereby the cancer is treated, cancer progression is stopped or slowed, and/or Stat3 is inhibited.

According to one aspect of the present invention, there are provided novel compositions comprising compounds represented by Formulae I-V, including compounds of Examples 1-88, their pharmaceutically acceptable salts, and pharmaceutical compositions containing them, or mixture thereof.

In some aspects, this disclosure provides for a method of screening a compound of Formulae I-V for their inhibitory effects intracellular Stat3 signaling (Stat3-binding activity) in cancer cells, the method comprising:

(a) contacting solid tumor cancer cells with a compound of Formulae I-V to form contacted solid tumor cancer cells,

(b) isolating nuclear extracts from the contacted solid tumor cancer cells,

(c) contacting the isolated nuclear extracts with a labelled oligonucleotide probe to form a labeled isolated nuclear extracts,

(d) performing EMSA analysis on the labeled isolated nuclear extracts,

(e) identifying the compound of Formulae I-V which results in the lowest relative concentration of the labeled isolated nuclear extracts as the most potent Stat3 inhibiting compound.

In some aspects, the solid tumor cancer cells are Human breast cancer cells. In some aspects, the Human breast cancer cells are selected from MDA-MB-231 or MDA-MB-468 cell lines. In some aspects, the compound of Formulae I-V is present at a concentration ranging from 0.5 to 10 μM. In some aspects, the labelled oligonucleotide probe is labeled with ³²P at one or a plurality of phosphate groups within the oligonucleotide. In some aspects, the labelled oligonucleotide probe is labeled with a fluorophore in place of a 5′ or 3′ phosphate, hydroxyl, or hydrogen on a base of the oligonucleotide.

The inventions described and claimed herein have many attributes and embodiments, including, but not limited to, those set forth, or described, or referenced, in this Brief Summary. It is not intended to be all-inclusive and the inventions described and claimed herein are not limited to, or by the features or embodiments identified in, this Brief Summary, which is included for purposes of illustration only and not restriction. Additional embodiments may be disclosed in the Detailed Description below.

### BRIEF DESCRIPTION OF THE FIGURES

FIGS. 1A-1N. Show the effects of aryl sulfonamido azetidine compounds on cell viability in vitro relative to control (untreated cells). The cells lines used were those of melanoma cells harboring aberrantly-active Stat3 (MDA-MB-231, MDA-MB-468) and counterpart that does not (MCF-7 and MCF-10A). Cell numbers were counted by trypan blue exclusion/phase-contrast microscopy every 34 h, and cell growth curve was plotted against concentration from which IC₅₀ values were derived. Cells in 6-well plates were treated once with 0.05% DMSO, or at the indicated concentrations (0.5, 1, 2, 3, 4, 5 and 10 μM if not otherwise so indicated) of the test compounds. FIG. 1A shows the MDA-MB-231 cell viability as a percentatge of control (DMSO vehicle only) for the compounds H172 or H182 after 72 hr of exposure of the compounds to the cells. FIG. 1B shows the MCF-10A cell viability for the compounds H172 or H182 after 72 hr of exposure of the compounds to the cells. FIG. 1C shows the MDA-MB-468 cell viability for the compounds H172 or H182 after 72 hr of exposure of the compounds to the cells. FIG. 1D shows the summary of the relative IC50 values (in micromolar) against cell lines harboring Stat3 activity compared to the cell lines without active Stat3, indicating that the compounds of this disclosure were more potent to cells harboring active Stat3. FIG. 1E shows the MDA-MB-231 cell viability for representative azetidine compounds of the invention (H205, H206, H207, H208, H209, H210, and H211) at 0, 1, and 3 micromolar concentration. FIG. 1F shows the MDA-MB-231 cell viability for a first experiment using a representative azetidine compound, H230, of the invention. FIG. 1G shows the MDA-MB-231 cell viability for a second experiment using a representative azetidine compound, H230, and H235, of the invention. FIG. 1H shows the MDA-MB-231 cell viability for a representative azetidine compound, H226, of the invention. FIG. 1I shows the MDA-MB-231 cell viability for a representative azetidine compound, H257, of the invention. FIG. 1J shows the MDA-MB-231 cell viability for a representative azetidine compound, H258, of the invention. FIG. 1K shows the MDA-MB-231 cell viability for representative azetidine compounds, H242, H246, and H250, of the invention. FIG. 1L shows the MDA-MB-231 cell viability for representative azetidine compounds, H277, H281, H283, H284, H286, and H288, of the invention. FIG. 1M shows the MDA-MB-231 cell viability for representative azetidine compounds, H266 and H267, of the invention. FIG. 1N shows the summary of EC50 values for MDA-MB-231 and/or MDA-MB-468 cell viability for representative azetidine compounds, H251, H260, H274, and H275, of the invention. The compound names are correlated with the structures provided in the Examples in Table 1.

FIGS. 2A-2B. show the in vivo efficacy of an aryl sulfonamido azetidine compound (H182) on the growth of subcutaneous MDA-MB-231 tumor xenografts. FIG. 2A shows the mouse model tumor volume as a function of time dosed (10 mg/kg every other day) treatment compared to vehicle control. (5 mice per group). The subjects were administered intraperitoneally. The results demonstrate that an aryl sulfonamido azetidine compound (H182) is effective in reducing the tumor growth volume relative to vehicle control. FIG. 2B shows the body weight of treated vs. untreated mice were equivalent throughout the treatment regime, indicating that the aryl sulfonamido azetidine compound did not result in weight loss of the subjects.

FIGS. 3A-3D. show the effect of aryl sulfonamido compounds described herein on Stat3 DNA-binding activity in vitro. FIG. 3A shows the Electrophoretic mobility shift assay (EMSA) analysis for a subset of the compounds of Examples 1-88 (correlated in Table 1) of Stat3 DNA-binding activity in nuclear extracts of equal total protein containing activated Stat3 pre-incubated with 0-100 μM of the indicated compounds for 30 min at room temperature prior to incubation with the radiolabeled hSIE probe that binds Stat3. Positions of Stat3:DNA complexes in gel are labeled; control lanes (0) represent nuclear extracts pre-treated with 10% DMSO. Data are representative of 1-3 independent determinations. FIG. 3B shows the Electrophoretic mobility shift assay (EMSA) analysis for a separate subset of the compounds of Examples 1-88 (correlated in Table 1). FIG. 3C shows the Electrophoretic mobility shift assay (EMSA) analysis for a separate subset of the compounds of Examples 1-88 (correlated in Table 1). FIG. 3D shows the Electrophoretic mobility shift assay (EMSA) analysis for a separate subset of the compounds of Examples 1-88 (correlated in Table 1).

FIGS. 4A-4B show the synergistic effects of an aryl sulfonamido azetidine compound (H169) and clinically used chemotherapy drugs on cell viability. Human triple-negative breast cancer cell line MDA-MB-231 (5000 cells/well) were seeded in 96-well plates. On the next day, cells were first treated with H169 at 1 μM or DMSO (control, 0.5% (v/v)) for 6 hours prior to treatment with an increasing concentrations of cisplatin or docetaxel, and incubated for a total of 72 hours. Cell proliferation and viability were measured by CyQuant NF Cell Proliferation Assay kit (ThermoFisher Scientific). Cell viability was normalized to the percentage of the control groups. FIG. 4A shows the synergistic effect of docetaxel and H169, wherein the combination of docetaxel with H169 resulted in a cancer cell death than docetaxel alone. In addition, the inventors have observed that the combination of docetaxel and H169 results in a higher rate of tumor size reduction than docetaxel alone or H169 alone, further establishing the synergistic effects of the methods of the present invention. FIG. 4B shows the synergistic effect of cisplatin and H169, wherein the combination of docetaxel with H169 resulted in a cancer cell death than cisplatin alone.

FIG. 5 shows the induction of apoptosis of human breast cancer cells using an aryl sulfonamido azetidine compound (H169). Human breast cancer cells, MDA-MB-231, were treated in culture with 3 micromolar concentration of H169 for 0-24 hours, whole-cell lysates prepared, and samples of equal total protein were subjected to SDS/PAGE-Western blotting analysis probing for pYSTAT3, STAT3, full-length PARP, cleaved PARP, and tubulin. Positions of proteins in gel are shown; control (“Con”, no H169) lane represents whole-cell lysates prepared from 0.05% DMSO-treated cells. Data are representative of 2 independent determinations. The data show that while STAT3 is significantly inhibited in the treated cells after 24 hr exposure, the tubulin and PARP concentrations are not affected much, indicating the selective STAT3 inhibition of the aryl sulfonamido azetidine compound H169.

### DETAILED DESCRIPTION

This description of the exemplary embodiments is intended to be read in connection with the accompanying drawings, which are to be considered part of the entire written description. Additionally, the section headings used herein are for organizational purposes only, and are not to be construed as limiting the subject matter described.

The present disclosure relates generally to novel, potent and selective aryl sulfonamide derivatized Stat3 inhibitors. Constituitively activated Stat3 has been found to play a role in cancerous cells and the substantially faster proliferation, invasiveness and rate of cancerous cells compared to cells of the non-cancerous origin. In some embodiments, the selective Stat3 inhibitors of this invention can suppress cancer cell growth, proliferation, survival, angiogenesis, migration/invasion and/or immunity. The inhibition of Stat3 can be achieved by inhibiting dimerization of Stat3.

Stat3:Stat3 protein complexes are mediated through reciprocal pTyr705-SH2 domain interactions. Most drugs targeting Stat3 include a phosphoryl group to mimic pTyr705. While the phosphate functionality is regarded as being essential to targeting the SH2 domain, it is unsuitable for drug discovery as it suffers from poor cell permeability and metabolic degradation. As described herein, it was surprisingly found that the compounds of Formulae I-V including compounds of Examples 1-88 are highly potent aryl sulfonamide derivatized Stat3 inhibitors with micromolar and sub-micromolar potency against some of the most aggressive brain cancer cells identified to this date.

The prevalence of constitutively-active Stat3 in human tumors places an increasing importance on the discovery of suitable Stat3-inhibitors as novel anticancer drugs; however, although many Stat3 inhibiting modalities have been reported, no Stat3 small-molecule inhibitor drug has yet reached to the clinic (Miklossy et al., Nat Rev Drug Discov 12:611-629 (2013)). As described herein, compounds of Formulae I-V including compounds of Examples 1-88, exhibit Stat3-inhibitory potency in vitro. As described herein, the compounds also show antitumor cell responses to breast cancer cells at low micromolar concentrations.

Substantive evidence demonstrates that aberrant Stat3 activity promotes cancer cell growth and survival, and induces tumor angiogenesis and metastasis. Inhibitors of Stat3 activation promote antitumor cell effects, although many of these have low potencies (See Turkson et al., Mol Cancer Ther 3:261-269 (2004); Turkson et al., J. Biol. Chem. 276:45443-45455 (2001); Garcia et al., Oncogene 20:2499-2513 (2001); Catlett-Falcone et al., Immunity 10:105-115 (1999); Mora et al., Cancer Res 62:6659-66 (2002); Niu et al., Oncogene 21:2000-2008 (2002); Wei et al., Oncogene 22:319-29 (2003); Xie et al., Oncogene 23:3550-60 (2004)).

The present disclosure is based on the surprising discovery that certain structurally distinct analogs of previously reported Stat3 inhibitors had unexpected and potentiated therapeutic activity. It was further discovered that difluorocyano substituents on the aryl sulfonamidyl moiety further increased potency. Mechanistic insight into the biological effects of select compounds of the invention as a Stat3 inhibitor is provided by the evidence disclosed herein of suppression of the constitutive expression of genes regulated by Stat3 genes, including Bcl-2, Bcl-xL, Cyclin D1, c-Myc, and Survivin, which control cell growth and survival (Song et al., Proc Natl Acad Sci USA. 102:4700-5 (2005); Zhang et al., Proc Natl Acad Sci USA 109:9623-8 (2012); Catlett-Falcone et al., Immunity 10:105-115 (1999); Gritsko et al., Clin Cancer Res. 12:11-9 (2006)). The inventors have developed potent and physicochemically acceptable compounds with proper selectivity by utilizing a rational, computer-aided molecule optimization and chemical synthesis approach to furnish potent and drug-like compounds. The inventors surprisingly discovered that the compounds of the present Examples strongly inhibited Stat3 DNA-binding activity in vitro, with an IC₅₀ of some exemplified embodiments of the present invention exhibiting as low as 0.283 micromolar. Altogether the present study provides evidence for the inhibition of constitutively-active Stat3 in malignant cells that lead to antitumor cell effects against human breast cancer cells in vitro.

### Definitions

General terms used in formula can be defined as follows; however, the meaning stated should not be interpreted as limiting the scope of the term per se.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which comprise oxygen, nitrogen, sulfur, or phosphorous, atoms replacing one or more carbons of the hydrocarbon backbone. The term “aromatic-alkyl” includes alkyl groups substituted with one or more aryl groups. The term “lower alkyl” as used herein refers to 4 or fewer carbons.

The term “aryl” includes groups with aromaticity, including 5- and 6-membered single-ring aromatic groups that may include from zero to four heteroatoms, as well as multicyclic systems with at least one aromatic ring. Examples of aryl groups include benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, aryl (substituted or unsubstituted as described herein), phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. In some embodiments, an aryl group can be substituted with an aryl group which is substituted with 1-5 halogens. Aryl groups can also be fused, or bridged, with alicyclic or heterocyclic rings which are not aromatic, so as to form a multicyclic system (e.g., tetralin, methylenedioxyphenyl).

As used herein, the term “alkylene” refers to divalent saturated aliphatic groups and includes both straight chain and branched chain groups.

As used herein, the term “alkenylene” refers to divalent aliphatic groups having a double bond and includes both straight chain and branched chain groups.

As used herein, the designation “Cy” represents a cyclohexyl moiety. The designation “Cp” represents a cyclopentyl moiety.

As used herein, the number of carbon atoms is depicted as either the range of carbon atoms listed by number in subscript (e.g., “C₃₋₁₀”) or the range of carbon atoms listed by letter and number in subscript (e.g., “C₃-C₁₀”).

As used herein, “cycloalkyl” or “carbocyclic” refers to a radical of a non-aromatic cyclic hydrocarbon group having from 3 to 10 ring carbon atoms (“C₃₋₁₀ cycloalkyl” or “C₃-C₁₀ cycloalkyl”) and zero heteroatoms in the non-aromatic ring system. In some embodiments, a cycloalkyl group has 3 to 8 ring carbon atoms (“C₃₋₈ cycloalkyl”). In some embodiments, a cycloalkyl group has 3 to 7 ring carbon atoms (“C₃₋₇ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), heptanyl (C₇), octanyl (C₈), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), and the like. As the foregoing examples illustrate, in certain embodiments, the cycloalkyl group is either monocyclic (“monocyclic cycloalkyl”) or polycyclic (e.g., containing a fused or ring system such as a bicyclic system (“bicyclic cycloalkyl”) or tricyclic system (“tricyclic cycloalkyl”)) and can be saturated or can contain one or more carbon-carbon double or triple bonds. “Cycloalkyl” also includes ring systems wherein the cycloalkyl ring, as defined above, is fused with one or more aryl or heteroaryl groups wherein the point of attachment is on the cycloalkyl ring, and in such instances, the number of carbons continue to designate the number of carbons in the carbocyclic ring system. Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heteroaryl” refers to a radical of a 5-14 membered monocyclic or polycyclic (e.g., bicyclic, tricyclic) 4n+2 aromatic ring system (e.g., having 6, 10, or 14 electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom or the ring that does not contain a heteroatom.

As used herein, the term “heterocyclyl” or “heterocycle” refers to a radical of a 5-12 membered monocyclic or polycyclic ring system, having ring carbon atoms and 1-4 ring heteroatoms provided in the ring system, wherein each heteroatom is independently selected from nitrogen, oxygen and sulfur. Heterocycles can include or exclude pyrazines, pyridazines, pyrimidines, lactones, lactams, and combinations thereof (e.g., a lactam which is also a pyrimidine).

As understood from the above, alkyl, alkenyl, alkylenyl, cycloalkyl, cycloalkenyl, heterocyclyl, aryl, and heteroaryl groups, as defined herein, are, in certain embodiments, optionally substituted. Optionally substituted refers to a group which may be substituted or unsubstituted. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present present disclosure contemplates any and all such combinations in order to arrive at a stable compound. For purposes of this present disclosure, heteroatoms such as nitrogen may have hydrogen substituents and/or any suitable substituent as described herein which satisfy the valencies of the heteroatoms and results in the formation of a stable moiety.

As used herein, the term “halo” or “halogen” refers to fluorine (fluoro, —F), chlorine (chloro, —Cl), bromine (bromo, —Br), or iodine (iodo, —I).

The term “prodrug” as used herein refers to a modified compound of Formulae I-V wherein an amino, carboxylic acid, or hydroxy functional group is further connected to a promoiety. In some embodiments “promoiety” refers to a species acting as a protecting group which masks a functional group within an active agent, thereby converting the active agent into a pro-drug. Typically, the promoiety will be attached to the drug via bond(s) that are cleaved by enzymatic or non-enzymatic means in vivo, thereby converting the pro-drug into its active form. In some embodiments the promoiety may also be an active agent. In some embodiments the promoiety may be bound to a compound of Formulae I-V.

In some embodiments the promoiety may include or exclude C1-C4 carboxylic acids, C1-C4 alcohols, C1-C4 aldehydes, C1-C4 ketones, a single amino acid or a peptide. In some embodiments, the promoiety is a single amino acid which is optionally protected on its functional groups. Methods of forming prodrugs by coupling the aforementioned promoieties to compounds of Formulae I-V can be performed by using conventional ester, amide, or acetal formation methods which are well-understood in the art. As a non-limiting example, a carboxylic acid functional group on a compound of Formulae I-V can be reacted with ethanol in the presence of EDC for form an ester.

In some embodiments, the promoiety is a targeting species. In some aspects, the promoiety is a substrate for an influx or efflux transporters on the cell membrane, for example those described in Gaudana, R. et al. The AAPS Journal, 12:3, 348-360 (2012), herein incorporated by reference. The promoiety can be, for example, chemically-linked biotin. The promoiety can be, for example, chemically-linked D-serine.

As used herein, a “subject” refers to an animal that is the object of treatment, observation or experiment. “Animal” includes cold- and warm-blooded vertebrates and invertebrates, such as fish, shellfish, reptiles and, in particular, mammals “Mammal” includes, without limitation, mice; rats; rabbits; guinea pigs; dogs; cats; sheep; goats; cows; horses; primates, such as monkeys, chimpanzees, apes, and prenatal, pediatric, and adult humans.

As used herein, “preventing” or “protecting” means preventing in whole or in part, or ameliorating, or controlling.

As used herein, the term “treating” refers to both therapeutic treatment and prophylactic, or preventative, measures, or administering an agent suspected of having therapeutic potential. The term includes preventative (e.g., prophylactic) and palliative treatment.

The term “pharmaceutically effective amount,” as used herein, means an amount of active compound, or pharmaceutical agent, that elicits the biological, or medicinal, response in a tissue, system, animal, or human that is being sought, which includes alleviation or palliation of the symptoms of the disease being treated and/or an amount sufficient to have utility and provide desired therapeutic endpoint. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. In some embodiments, the drug is cytostatic and/or cytotoxic to prevent growth and/or kill existing cancer cells. For cancer therapy, efficacy can be measured, e.g., by assessing the time to disease progression and/or determining the response rate.

The term “pharmaceutically acceptable,” as used herein, means that the substance or composition must be compatible chemically and/or toxicologically, with the other ingredients comprising a formulation, and/or the mammal being treated therewith.

The term “cancer” refers to, or describes, the physiological condition in mammals that is characterized by unregulated cell growth and/or hyperproliferative activities. A “tumor” comprises one or more cancerous cells. Examples of cancer include, but are not limited to, carcinoma, lymphoma, blastoma, sarcoma, and leukemia or lymphoid malignancies. In one embodiment, the cancer is a solid tumor. More particular examples of such cancers include breast cancer, cervical cancer, ovarian cancer, bladder cancer, endometrial or uterine carcinoma, prostate cancer, glioma and other brain or spinal cord cancers, squamous cell cancer (e.g., epithelial squamous cell cancer), lung cancer, including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, hepatoma, colon cancer, rectal cancer, colorectal cancer, salivary gland carcinoma, kidney or renal cancer, vulval cancer, thyroid cancer, hepatic carcinoma, anal carcinoma, penile carcinoma, as well as head and neck cancer. In one embodiment, the treatment comprises treatment of solid tumors. In one embodiment, the tumors comprises sarcomas, carcinomas or lymphomas.

In some embodiments, the cancer can include or exclude: brain tumors, such as gliomas, medulloblastomas, cerebral menangiomas, pancreatic cancer, malignant melanoma, multiple myeloma, lymphomas, including anaplastic large T cell lymphoma, sezary syndrome, EBV-related Burkitt's Lymphoma, HSV Saimiri-dependent (T Cell), cutaneous T cell lymphoma, mycosis fungoides, leukemia, including HTLV-I dependent leukemia, erythroleukemia, acute lymphocytic leukemia (ALL), chronic lymphocytic leukemia (CLL), acute myelogenous leukemia (AML), chronic myelogenous leukemia (CML), megakaryocytic leukemia, and large granula lymphocyte (LGL) leukemia, thyroid cancer, brain cancer, skin cancer, lung cancer, and kidney cancer. In some embodiments the cancer can include or exclude renal cell carcinoma, pancreatic adenocarcinoma, ovarian carcinoma or Hodgkin Lymphoma.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytoxic/antitumor antibiotics, topoisomerase inhibitors, antibodies, photosensitizers, and kinase inhibitors. Chemotherapeutic agents include compounds used in “targeted therapy” and conventional chemotherapy. Examples of chemotherapeutic agents include: trastuzumab (HERCEPTIN®, Genentech), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), pemetrexed (ALIMTA®, Eli Lilly), temozolomide (4-methyl-5-oxo-2,3,4,6,8-pentazabicyclo[4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate, or inhibit, hormone action on tumors, such as anti-estrogens and selective estrogen receptor modulators (SERMs), including, e.g., tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, e.g., 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, e.g., PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, e.g., ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents, in combination with the compounds of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

A “metabolite” is a product produced through metabolism in the body of a specified compound, or salt thereof. Metabolites of a compound may be identified using tests such as those described herein. Such products may result e.g., from the oxidation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

The phrase “pharmaceutically acceptable salt” as used herein, refers to pharmaceutically acceptable organic, or inorganic, salts of a compound of the invention. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, and pamoate (i.e., 1,1′-methylene-bis(2-hydroxy-3-naphthoate)) salts. A pharmaceutically acceptable salt may involve the inclusion of another molecule, such as an acetate ion, a succinate ion, or other counter ion. In some embodiments, the counter ion is any organic, or inorganic, moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

In some embodiments, when the compound of the invention is a base, the desired pharmaceutically acceptable salt is prepared by any suitable method available in the art, e.g., treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

In some embodiments, when the compound of the invention is an acid, the desired pharmaceutically acceptable salt is prepared by any suitable method, e.g., treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A “solvate” refers to an association, or complex, of one or more solvent molecules and a compound of the invention. Examples of solvents that form solvates include, but are not limited to, water, isopropanol, ethanol, methanol, DMSO, ethylacetate, acetic acid, and ethanolamine

### Administration of Formulae I-V Compounds

In some embodiments, the Formulae I-V compounds of the invention, including compounds of Examples 1-88 are administered by any route appropriate to the condition to be treated. Suitable routes can include or exclude oral, parenteral (including subcutaneous, intramuscular, intravenous, intraarterial, intradermal, intrathecal and epidural), intraperitoneal (IP), transdermal, rectal, nasal, topical (including buccal and sublingual), vaginal, intrapulmonary and intranasal. In some embodiments, for local treatment, the compounds are administered by intratumor administration, including perfusing or otherwise contacting the tumor with the inhibitor. It will be appreciated that the preferred route may vary with, e.g., the condition of the recipient. In some embodiments, where the compound is administered orally, it is formulated as a pill, capsule, tablet, etc., with a pharmaceutically acceptable carrier or excipient. In some embodiments, where the compound is administered parenterally, it is formulated with a pharmaceutically acceptable parenteral vehicle, and in a unit dosage injectable form, as described herein.

All descriptions with respect to dosing, unless otherwise expressly stated, apply to the compounds of the invention, including compounds of Formulae I-V.

The compounds of Formulae I-V or prodrugs thereof of the invention can be dosed, administered or formulated as described herein.

As will be appreciated, the dose of compounds of Formulae I-V or prodrugs thereof administered, the period of administration, and the general administration regime may differ between subjects depending on such variables as the target site to which it is to be delivered, the severity of any symptoms of a subject to be treated, the type of disorder to be treated, size of unit dosage, the mode of administration chosen, and the age, sex and/or general health of a subject and other factors known to those of ordinary skill in the art.

Data obtained from cell culture assays and animal studies can be used in formulating a range of dosages for use in humans. The dosage may vary within this range depending upon the dosage form employed and the route of administration utilized. For any agent used in the method of the invention, the therapeutically effective dose can be estimated initially from cell culture assays. A dose may be formulated in cell cultures or animal models to achieve a cellular concentration range that includes the IC50 (i.e., the concentration of the test compound that achieves a half-maximal inhibition of symptoms) as determined in cell culture. Such information can be used to more accurately determine useful doses in humans. The exact formulation, route of administration and dosage can be chosen by the individual physician in view of the patient's condition. (See, e.g., Fingl et al., 1975, In: The Pharmacological Basis of Therapeutics, Ch. 1, p. 1). The dosage can be determined from the concentration of the amount administered, expected mass of the animal model tested (200-300 g per rat for adult Wistar rats), to determine the dose in units of mg/kg from concentration (micromolar) administered or amount (mg) administered.

In some embodiments, a dose to treat human patients is from about 1 mg to about 1000 mg of compound of Formulae I-V, including compounds of Examples 1-88. The dose is from about 1 mg, 2 mg, 2.5 mg, 4 mg, 5 mg, 7.5 mg, 10 mg, 12.5 mg, 15 mg, 17, 5 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg of a compound of Formulae I-V (including compounds of Examples 1-88), or any dose ranging between any two of those doses.

In some embodiments, a dose is administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. In some embodiments, for orally administered doses, the pill, capsule, or tablet is ingested daily or less frequently for a specified period of time. In some embodiments, the regimen is repeated for a number of cycles of therapy.

Pharmaceutical compositions are provided for single, combined, simultaneous, separate, sequential, or sustained administration. In one embodiment, a composition comprising one or more compounds of Formulae I-V or prodrugs thereof is administered at in or more desired doses at one or more times. In another embodiment, a composition comprising one or more compounds of Formulae I-V or prodrugs thereof is administered about the same time as a chemotherapeutic agent. When the two compositions are administered at different times, they may be administered within, for example, 30 minutes, 1 hour, 1 day, 1 week, or 1 month part, or any time interval between any two of the recited time periods. Doses may be administered QD, BID, TID, QID, or in weekly doses, e.g., QIW, BIW QW. They may also be administered PRN, and hora somni.

### Manufacture and Stability

In some embodiments, the formulations of this invention are substantially pure. By substantially pure is meant that the formulations comprise less than about 10%, 5%, or 1%, and preferably less than about 0.1%, of any impurity. In some embodiments the total impurities, including metabolities of the compounds of Formulae I-V will be not more than 15%. In some embodiments the total impurities, including metabolities of the compounds of Formulae I-V, will be not more than 12%. In some embodiments the total impurities, including metabolities of the compounds of Formulae I-V will be not more than 11%. In other embodiments the total impurities, including metabolities of compounds of Formulae I-V will be not more than 10%.

In some embodiments, the purity of the formulations of this invention may be measured using a method selected from anion exchange HPLC (AEX-HPLC) or mass spectrometry. Mass spectrometry may include LC/MS, or LC/MS/MS. In some embodiments, the method used to measure the impurity may comprise both AEX-HPLC and LC/MS.

Sterile compositions comprising the compounds of Formulae I-V or prodrugs thereof of this invention prepared using aseptic processing by dissolving the compound in the formulation vehicle. In one embodiment, the formulation may also be sterilized by filtration. Excipients used in the manufacture of of the formulations of this invention are widely used in pharmaceutical products and released to pharmacopeial standards.

### Methods of Treatment with Formulae I-V Compounds

Compounds of Formulae I-V, including compounds of Examples 1-88, are useful for treating hyperproliferative diseases, conditions and/or disorders including, but not limited to, cancer. Accordingly, an embodiment of this invention includes methods of treating, or preventing, diseases or conditions that can be treated or prevented by inhibiting Stat3. In one embodiment, the method comprises administering to a subject, in need thereof, a therapeutically effective amount of a compound of Formula I, or pharmaceutically acceptable salt thereof. In one embodiment, a human patient is treated with a compound of Formulae I-V, including compounds of Examples 1-88 and a pharmaceutically acceptable carrier, adjuvant, or vehicle, wherein said compound of Formulae I-V, including compounds of Examples 1-88, is present in an amount to treat cancer and/or detectably inhibit Stat3 activity.

In some embodiments, the methods of this inventions can treat Cancers which can include or exclude: glioma, glioblastoma, neuroblastoma, breast, ovary, cervix, prostate, testis, genitourinary tract, esophagus, larynx, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In some embodiments, compounds of Formulae I-V, including compounds of Examples 1-88, are useful for in vitro, in situ, and in vivo diagnosis or treatment of mammalian cells, organisms, or associated pathological conditions, such as hyperproliferative disease and/or cancer.

In some embodiments, compounds of Formulae I-V, including compounds of Examples 1-88, are useful for treating conditions of the brain and central nervous system which require transport across the blood-brain barrier. Certain compounds of Formulae I-V, including compounds of Examples 1-88, have favorable penetrant properties for delivery to the brain. In some embodiments, compounds of Formulae I-V, including compounds of Examples 1-88 are used to treat disorders of the brain which can include or exclude metastatic and primary brain tumors, such as glioblastoma and melanoma.

In some embodiments, compounds of Formulae I-V, including compounds of Examples 1-88 are useful for treating eye cancers by localized delivery to the eye. Certain compounds of Formulae I-V, including compounds of Examples 1-88 have favorable properties for delivery to, and uptake into, the eye. In some embodiments, selected compounds of Formulae I-V, including compounds of Examples 1-88 enhance efficacy and extend duration of response for treatment of wet AMD in combination with ranibizumab (LUCENTIS®, Genentech, Inc.) and bevacizumab (AVASTIN®, Genentech, Inc.).

Another embodiment of this invention includes a compound of this invention for use in the treatment of the diseases or conditions described herein in a subject, e.g., a human, suffering from such disease or condition. Also provided is the use of a compound of this invention in the preparation of a medicament for the treatment of the diseases and conditions described herein in a warm-blooded animal, such as a mammal, e.g. a human, suffering from such disorder.

### Pharmaceutical Formulation/Compositions and Uses

In order to use a compound of Formulae I-V, including compounds of Examples 1-88 for the therapeutic treatment (including prophylactic treatment) of mammals including humans, in some embodiments the compound is formulated in accordance with standard pharmaceutical practice as a pharmaceutical composition. According to this embodiment of the invention, there is provided a pharmaceutical composition comprising a compound of this invention in association with a pharmaceutically acceptable diluent or carrier.

In some embodiments, a formulation of the present invention is prepared by mixing a compound of Formulae I-V, and a carrier, diluent or excipient. Suitable carriers, diluents and excipients include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

In some embodiments, formulations of the present invention are prepared using dissolution and mixing procedures. For example, the bulk drug substance (i.e., compound of the present invention or stabilized form of the compound of Formulae I-V, including compounds of Examples 1-88 (e.g., complex with a cyclodextrin derivative or other complexation agent) is dissolved in a suitable solvent in the presence of one or more of the excipients described above. In some embodiments, the compound of the present invention is formulated into pharmaceutical dosage forms to provide an easily controllable dosage of the drug and to enable patient compliance with the prescribed regimen.

In some embodiments, the pharmaceutical composition (or formulation) for application is packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In some embodiments, pharmaceutical formulations of the compounds of the present invention are prepared for various routes and types of administration. In some embodiments, a compound of Formulae I-V, including compounds of Examples 1-88 having the desired degree of purity is mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. In some embodiments, formulation is conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compound of this invention for use herein is preferably sterile. In particular, formulations to be used for in vivo administration must be sterile. Such sterilization is readily accomplished by filtration through sterile filtration membranes.

In some embodiments, the compound is stored as a solid composition, a lyophilized formulation or as an aqueous solution (e.g. in saline).

In some embodiments, the pharmaceutical compositions of the invention comprising a compound of Formulae I-V, including compounds of Examples 1-88 is formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. In addition to the compounds and salt forms provided herein, the invention includes pharmaceutical compositions, including tablets, capsules, solutions, and suspensions for parenteral and oral delivery forms and formulations, comprising a pharmaceutically acceptable carrier and therapeutically effective amounts of one or more of the aryl sulfonamide derivatized Stat3 inhibitors herein provided. Stat3 inhibitor pharmaceutical compositions can include salts and hydrates.

In human and animal therapy for the treatment of cancer, for example in the treatment of cancer and other related disorders, diseases and conditions noted herein, the compounds and their crystal forms described and provided herein, their pharmaceutically acceptable salts, and pharmaceutically acceptable solvates of either entity, can be administered alone, but will generally be administered in admixture with a pharmaceutical carrier selected with regard to the intended route of administration and standard pharmaceutical practice. Preferably, they are administered orally in the form of tablets comprising pharmaceutically acceptable excipients, such as starch or lactose, or in capsules or ovules either alone or in admixture with excipients, or in the form of elixirs, solutions or suspensions comprising flavouring or colouring agents. They can also be injected parenterally, for example, intravenously, intramuscularly or subcutaneously. For parenteral administration, they are best used in the form of a sterile aqueous solution which may contain other substances, for example enough salts or monosaccharides to make the solution isotonic with blood. For buccal or sublingual administration they may be administered in the form of tablets or lozenges which can be formulated in a conventional manner

In some embodiments, the initial pharmaceutically effective amount of the compound of Formulae I-V, including compounds of Examples 1-88 administered parenterally per dose will be in the range of about 0.001-10 mg/kg, 0.001-0.01, or 0.01-1.0, or 1.0 to 10.0 or 10.0 to 100.0 mg/kg. In some embodiments, the amount of the compound of Formulae I-V, including compounds of Examples 1-88 administered parenterally per dose is about 0.05 to 5 mg/kg of patient body weight per day, with the initial range of compound used being 0.05 to 10 mg/kg/day. In some embodiments, a dose is about 1 mg to about 30.0 mg once, twice or four times a day of the compound. In some embodiments, the dose is about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.9, 3.0, 3.1, 3.2, 3.3, 3.4, 3.5, 3.6, 3.7, 3.8, 3.9, 4.0, 4.1, 4.5, 4.9 or about 5.0 mg/kg, or any range in between any two of the recited doses. In some embodiments the dose will be 0.08 mg/kg to about 0.5 mg/kg, from about 0.08 to about 0.24 mg/kg, or from about 0.24 to about 0.5 mg/kg. In some embodiments, the effective dose of the Stat3 inhibitor is given in one or more doses. In some embodiments, a therapeutically effective amount is selected from: 0.08, 0.24, or 0.5 mg/kg for each dose.

Acceptable diluents, carriers, excipients and stabilizers are nontoxic to recipients at the dosages and concentrations employed, and include saline and/or buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, e.g., by coacervation techniques or by interfacial polymerization, e.g., hydroxymethylcellulose or gelatin-microcapsules and poly-(methylmethacylate) microcapsules, respectively, in colloidal drug delivery systems (e.g., liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, sustained-release formulations of compounds of Formulae I-V, including compounds of Examples 1-88 are prepared. In some embodiments, sustained-release formulations can include or exclude semipermeable matrices of solid hydrophobic polymers comprising a compound of Formulae I-V, including compounds of Examples 1-88 which matrices are in the form of shaped articles, e.g., films, or microcapsules. In some embodiments, examples of sustained-release matrices can include or exclude polyesters, hydrogels (e.g., poly(2-hydroxyethyl-methacrylate), or poly(vinyl alcohol)), polylactides (U.S. Pat. No. 3,773,919 herein incorporated by reference), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations of this disclosure include those suitable for the administration routes detailed herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

In some embodiments, formulations of a compound of Formulae I-V, including compounds of Examples 1-88 suitable for oral administration are prepared as discrete units such as pills, capsules, cachets or tablets each comprising a predetermined amount of a compound of Formulae I-V, including compounds of Examples 1-88.

In some embodiments, compressed tablets are prepared by compressing in a suitable machine the active ingredient in a free-flowing form such as a powder or granules, optionally mixed with a binder, lubricant, inert diluent, preservative, surface active or dispersing agent. In some embodiments, molded tablets are made by molding in a suitable machine a mixture of the powdered active ingredient moistened with an inert liquid diluent. In some embodiments, the tablets are coated or scored and are formulated so as to provide slow or controlled release of the active ingredient therefrom.

In some embodiments, the formulations are prepared for oral use in the format which can include or exclude: tablets, troches, lozenges, aqueous or oil suspensions, dispersible powders or granules, emulsions, hard or soft capsules, e.g., gelatin capsules, syrups or elixirs. In some embodiments, formulations of compounds of Formulae I-V, including compounds of Examples 1-88 intended for oral use are prepared for the manufacture of pharmaceutical compositions and such compositions may contain one or more agents including sweetening agents, flavoring agents, coloring agents and preserving agents, in order to provide a palatable preparation. Tablets comprising the active ingredient in admixture with non-toxic pharmaceutically acceptable excipient which are suitable for manufacture of tablets are acceptable. These excipients can include or exclude inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. In some embodiments, tablets are uncoated or coated by techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, for treatment of the eye or other external tissues, e.g., mouth and skin, the formulations are applied as a topical ointment or cream comprising the active ingredient(s) in an amount of, e.g., 0.075 to 20% w/w. When formulated in an ointment, the active ingredients are employed with either a paraffinic or a water-miscible ointment base. In some embodiments, the active ingredients are formulated in a cream with an oil-in-water cream base.

In some embodiments, the aqueous phase of the cream base can include or exclude a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. In some embodiments, the topical formulations include a compound which enhances absorption or penetration of the active ingredient through the skin or other affected areas. Examples of such dermal penetration enhancers include dimethyl sulfoxide and related analogs.

In some embodiments, the oily phase of the emulsions of this invention is constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of Formulae I-V compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

In some embodiments, the pharmaceutical compositions of compounds of Formulae I-V, including compounds of Examples 1-88 are in the form of a sterile injectable preparation, such as a sterile injectable aqueous or oleaginous suspension. In some embodiments, the suspension is formulated according to methods using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol prepared as a lyophilized powder. In some embodiments, the acceptable vehicles and solvents that are employed can include or exclude: water, Ringer's solution (including Ringer's lactate solution), Hartmann's solution, Tyrode's solution, and isotonic sodium chloride solution. In some embodiments, sterile fixed oils are employed as a solvent or suspending medium. For this purpose any bland fixed oil is employed including synthetic mono- or diglycerides. In some embodiments, fatty acids such as oleic acid is used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). In some embodiments, the pharmaceutical composition is prepared to provide easily measurable amounts for administration. For example, an aqueous solution intended for intravenous infusion may contain from about 10 to 10,000 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, about 0.5 to 10% w/w, or about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

In some embodiments, formulations for rectal administration are presented as a suppository with a suitable base comprising, e.g. cocoa butter or a salicylate.

Formulations suitable for intrapulmonary or nasal administration have a particle size, e.g. in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. In some embodiments, formulations suitable for aerosol or dry powder administration are prepared and delivered with other therapeutic agents such as compounds heretofore used in the treatment or prophylaxis disorders as described herein.

In some embodiments, formulations suitable for vaginal administration are presented as pessaries, tampons, creams, gels, pastes, foams or spray formulations comprising the active ingredient and pharmaceutically acceptable carriers.

In some embodiments, the formulations are packaged in unit-dose or multi-dose containers, e.g. sealed ampoules and vials, and are stored in a freeze-dried (lyophilized) condition requiring only the addition of the sterile liquid carrier, e.g., water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

In some embodiments, the compounds of Formulae I-V, including compounds of Examples 1-88 are employed alone, or in combination with other therapeutic agents, for the treatment of a disease or disorder described herein, such as a hyperproliferative disorder (e.g., cancer). In certain embodiments, a compound of Formulae I-V, including compounds of Examples 1-88 is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of Formulae I-V, including compounds of Examples 1-88 such that they do not adversely affect each other. Such compounds are suitably present in combination in amounts that are effective for the purpose intended. In one embodiment, a composition of this invention comprises a compound of Formulae I-V, including compounds of Examples 1-88 in combination with a chemotherapeutic agent such as described herein.

In some embodiments, the combination therapy is administered as a simultaneous or sequential regimen. In some embodiments, when administered sequentially, the combination is administered in two or more administrations. The combined administration includes coadministration, using separate formulations or a single pharmaceutical formulation, and consecutive administration in either order, wherein preferably there is a time period while both (or all) active agents simultaneously exert their biological activities.

In some embodiments, suitable dosages for any of the above coadministered agents are those presently used and can be lowered due to the combined action (synergy) of the newly identified agent and other chemotherapeutic agents or treatments.

The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes, separate pills or capsules, or separate infusions. In general, during alternation therapy, an effective dosage of each active ingredient is administered sequentially, i.e., serially, whereas in combination therapy, effective dosages of two or more active ingredients are administered together.

In a particular embodiment of anti-cancer therapy, a compound of Formulae I-V, including compounds of Examples 1-88 or a stereoisomer, geometric isomer, tautomer, solvate, metabolite, or pharmaceutically acceptable salt or prodrug thereof, is combined with other chemotherapeutic, hormonal or antibody agents such as those described herein, as well as combined with surgical therapy and radiotherapy. Combination therapies according to the present invention thus comprise the administration of at least one compound of Formulae I-V, including compounds of Examples 1-88 or a solvate, metabolite, or pharmaceutically acceptable salt or prodrug thereof, and the use of at least one other cancer treatment method. The amounts of the compound(s) of Formulae I-V, including compounds of Examples 1-88 and the other pharmaceutically active chemotherapeutic agent(s) and the relative timings of administration will be selected in order to achieve the desired combined therapeutic effect.

### Metabolites of Formulae I-V Compounds

Also falling within the scope of this invention are the in vivo metabolic products of Formulae I-V, including compounds of Examples 1-88 described herein. Such products may result, e.g., from the condensation, oxidation, reduction, hydrolysis, amidation, deamidation, esterification, deesterification, enzymatic cleavage, and the like, of the administered compound. Accordingly, the invention includes metabolites of compounds of Formulae I-V, including compounds of Examples 1-88 including compounds produced by a process comprising contacting a compound of this invention with a mammal for a period of time sufficient to yield a metabolic product thereof.

In some embodiments, metabolite products are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined by an analytical chemistry method, e.g., by MS, LC/MS or NMR analysis. The metabolite products, so long as they are not otherwise found in vivo, may be useful in diagnostic assays for therapeutic dosing of the compounds of the invention.

### Articles of Manufacture/Kits

In another embodiment of the invention, an article of manufacture, or “kit,” containing materials useful for the treatment of the diseases and disorders described above is provided. The kit contains a composition comprising a compound of Formulae I-V, including compounds of Examples 1-88. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container may hold a compound of Formulae I-V, including compounds of Examples 1-88, or a composition thereof which is effective for treating the condition and may have a sterile access port (e.g., the container is an intravenous solution bag or a vial having a stopper pierceable by a hypodermic injection needle). At least one active agent in the composition is a compound of Formulae I-V, including any of the compounds of Examples 1-88. The label or package insert indicates that the composition is used for treating the condition of choice, such as cancer. In addition, the label or package insert may indicate that the patient to be treated is one having a disorder such as a hyperproliferative disorder. In one embodiment, the label or package inserts indicates that the composition comprising a compound of Formulae I-V, including any of the compounds of Examples 1-88, is used to treat a disorder resulting from abnormal cell growth. The label or package insert also indicates that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution (including Ringer's lactate solution), Tyrode's solution, Hellmann's solution, and dextrose solution. In some embodiments, the article of manufacture includes or excludes other buffers, diluents, filters, needles, and syringes.

The kit may further comprise directions for the administration of the compound of Formulae I-V, including compounds of Examples 1-88 and, if present, the second pharmaceutical formulation. For example, if the kit comprises a first composition comprising a compound of Formulae I-V, including compounds of Examples 1-88, and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formulae I-V, including compounds of Examples 1-88, such as tablets or capsules. Such a kit preferably includes a number of unit dosages. Such kits can include a card having the dosages oriented in the order of their intended use. An example of such a kit is a “blister pack.” Blister packs are well known in the packaging industry and are widely used for packaging pharmaceutical unit dosage forms. If desired, a memory aid can be provided, e.g. in the form of numbers, letters, or other markings or with a calendar insert, designating the days in the treatment schedule in which the dosages can be administered.

According to one embodiment, a kit may comprise (a) a first container with a compound of Formulae I-V, including compounds of Examples 1-88 contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution (including Ringer's lactate solution), Hellmann's solution, Tyrode's solution, and dextrose solution. It may further include other materials desirable from a commercial and user standpoint, including other buffers, diluents, filters, needles, and syringes.

In certain other embodiments wherein the kit comprises a composition of Formulae I-V, including compounds of Examples 1-88 and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

The invention includes an article of manufacture comprising packaging material containing one or more dosage forms containing a Stat3 inhibitor provided herein, wherein the packaging material has a label that indicates that the dosage form can be used for a subject having or suspected of having or predisposed to any of the diseases, disorders and/or conditions described or referenced herein. Such dosage forms include, for example, tablets, capsules, solutions and suspensions for parenteral and oral delivery forms and formulations.

In yet another aspect of this invention is a kit comprising (a) at least one Stat3 inhibitor described herein, or salt or crystal thereof, and a pharmaceutically acceptable carrier, excipient and/or additive in a unit dosage form, and (b) means for containing the unit form. Since the present invention has an aspect that relates to the treatment of the disease/conditions described herein with a combination of active ingredients, the invention also relates to combining separate pharmaceutical compositions in kit form. A kit may contain a pharmaceutical composition comprising a Stat3 inhibitor, or salt or crystal thereof, as provided herein, either alone or together with a second compound as described herein.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Synthesis of Stat3 Inhibitors

The reaction schemes in Examples 1-88 show exemplary reaction schemes for the preparation of selected Stat3 inhibitor compounds of this invention, which may include a Stat3 inhibitor salt.

### Compositions

In some embodiments, this disclosure provides for compositions comprising a compound of Formula I-V, including compounds of Examples 1-88, and a pharmaceutically acceptable excipient. In some embodiments, the pharmaceutically acceptable excipient can include or exclude organic acids or salts thereof.

Organic acids include both aliphatic and aromatic carboxylic acids and include, for example, aliphatic monocarboxylic acids, aliphatic dicarboxylic acids, aliphatic tricarboxylic acids, aromatic monocarboxylic acids, aromatic dicarboxylic acids, and aromatic tricarboxylic acids.

Aliphatic carboxylic acids may be saturated or unsaturated. Suitable aliphatic carboxylic acids include those having from 2 to about 10 carbon atoms.

Aliphatic monocarboxylic acids include saturated aliphatic monocarboxylic acids and unsaturated aliphatic monocarboxylic acids. Examples of saturated monocarboxylic acids include acetic acid, propronic acid, butyric acid, valeric acid, caproic acid, enanthic acid, caprylic acid, pelargonic acid, and caprynic acid. Examples of unsaturated aliphatic monocarboxylic acids include acrylic acid, propiolic acid, methacrylic acid, crotonic acid and isocrotonic acid.

Aliphatic dicarboxylic acids include saturated aliphatic dicarboxylic acids and unsaturated aliphatic dicarboxylic acids. Examples of saturated aliphatic dicarboxylic acids include oxalic acid, malonic acid, succinic acid, glutaric acid, adipic acid, pimelic acid, suberic acid, azelaic acid, and sebacic acid. Examples of unsaturated aliphatic dicarboxylic acids include maleic acid, fumaric acid, citraconic acid, mesaconic acid, itaconic acid and the like.

In certain aspects, crystalline aryl sulfonamide derivatized Stat3 inhibitors and salts thereof are described. These include crystalline Stat3 inhibitor maleate, Stat3 inhibitor fumarate, and Stat3 inhibitor succinate. Different Stat3 inhibitor crystals include those comprising the geometric structures, unit cell structures, and structural coordinates.

Also described are Stat3 inhibitor salts of high purity, methods for their preparation, and dosage forms including Stat3 inhibitor salts.

The pharmaceutical compositions may include, for example, one or more pharmaceutically acceptable excipients, carriers, and/or additives suitable for oral or parenteral administration.

The product formed by the described processes is substantially pure, that is, substantially free from any other compounds. Preferably, it contains less than 10% impurities, and more preferably, less than about 5% impurities, and even more preferably, less than about 1% impurities. The product thus formed is also preferably substantially pure, i.e., contains less than 10% impurity, more preferably less than 5% impurity, and still more preferably less than 1% impurity. The present invention also includes a substantially pure anhydrous crystalline form of Stat3 inhibitor disuccinate. The term “substantially pure” means that a sample of the relevant anhydrous crystalline form of Stat3 inhibitor disuccinate contains more than 90% of a single polymorphic form, preferably more than 95% of a single polymorphic form, and still more preferably more than 99% of a single polymorphic form.

### Doses

In some embodiments, a therapeutically effective amount of the compounds herein and their pharmaceutically acceptable salts and solvates, is from about 1 mg to about 1000 mg of Formulae I-V compounds including compounds of Examples 1-88. The dose is from about 1 mg, 2 mg, 2.5 mg, 4 mg, 5 mg, 7.5 mg, 10 mg, 12.5 mg, 15 mg, 17, 5 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg of a compound of Formulae I-V including compounds of Examples 1-88 or any dose ranging between any two of those doses.

In some embodiments, a dose is about 1 mg to about 30.0 mg once, twice or four times a day of the compound. In some embodiments, the dose is about 0.05, 0.06, 0.07, 0.08, 0.09, 0.1, 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.9, 3.0, 3.1, 3.2, 3.3, 3.4, 3.5, 3.6, 3.7, 3.8, 3.9, 4.0, 5.0, 6.0, 7.0, 8.0, 9.0, 10.0, 11.0, 12.0, 13.0, 14.0, 15.0, 16.0, 17.0, 18.0, 19.0, 20.0, 22.5, 25.0, 27.5, 30.0, 32.5, 35.0, 37.5, or about 40.0 mg/kg, or any range in between any two of the recited doses. In some embodiments the dose will be 0.08 mg/kg to about 0.5 mg/kg, from about 0.08 to about 0.24 mg/kg, or from about 0.24 to about 0.5 mg/kg. In some embodiments, the effective dose of the aryl sulfonamide derivatized Stat3 inhibitor is given in one or more doses. In some embodiments, the therapeutically is selected from: 0.08, 0.24, and 0.5 mg/kg for each dose.

In some embodiments, a daily dosage level of the compounds herein, and their pharmaceutically acceptable salts and solvates, is from about 1 mg to about 5 g per day, or up to about 50 g per day (in single or divided doses). Other therapeutically effective dose ranges include, for example, from about 5 mg to about 25 mg, from about 5 mg to about 15 mg, from about 4 mg to about 35 mg, from about 35 mg to about 50 mg, from about 50 mg to about 100 mg, from about 100 mg to about 200 mg, from about 200 mg to about 500 mg, or from about 500 mg to about 1000 mg per day. In some embodiments, the total dose is selected from: 1 mg BID, 2 mg BID, 3 mg BID, 4 mg BID, 5 mg BID, 6 mg BID, 7 mg BID, 8 mg BID, 9 mg BID, 10 mg BID, 20 mg BID, 30 mg BID, 40 mg BID, 50 mg BID, 60 mg BID, 70 mg BID, 80 mg BID, 90 mg BID, 100 mg BID, 110 mg BID, 120 mg BID, 130 mg BID, 140 mg BID, 150 mg BID, 160 mg BID, 170 mg BID, 180 mg BID, 190 mg BID, 200 mg BID, 250 mg BID, 300 mg BID, or any total dose range between any of the aforementioned dose values.

Compounds described herein, and their pharmaceutically acceptable salts and solvates, will also be effective at doses in the order of 1/10, 1/50, 1/100, 1/200, 1/300, 1/400, 1/500 and even 1/1000 of those described herein.

In some embodiments of the invention, a therapeutically effective amount is the amount effective to elicit a plasma concentration of the compounds provided herein, and their pharmaceutically acceptable salts and solvates, from about 0.01 mg/L to about 20 mg/L, about 0.01 mg/L to about 15 mg/L, about 0.1 mg/L to about 10 mg/L, about 0.5 mg/L to about 9 mg/L, about 1 mg/L to about 8 mg/L, about 2 mg/L to about 7 mg/L or about 3 mg/L to about 6 mg/L.

In some embodiments, the doses described herein are administered in a single dose or multiple doses. In some embodiments, the doses are administered once, twice, three, four or more times a day, or one, two, three, four, five, or six times per week.

The physician will determine the actual dosage which will be most suitable for an individual patient, and it will vary with the age, weight and response of the particular patient. The above dosages are exemplary of the average case; there can, of course, be individual instances where higher or lower dosage ranges are merited, and such are within the scope of this invention.

Generally, in humans, IP administration of the compounds of the invention is the preferred route. A preferred oral dosing regimen in cancer treatment for a typical man is from about 1 mg to about 1000 mg per day of compound when required. Preventative doses are lower, from about about 0.3-100 mg to about 1-50 mg per day.

For veterinary use, a compound provided herein, or a veterinarily acceptable salt thereof, or a veterinarily acceptable solvate of either entity, is administered as a suitably acceptable formulation.

Thus the invention provides a pharmaceutical composition comprising an aryl sulfonamide derivatized Stat3 inhibitor, which may include a Stat3 inhibitor salt compound provided herein, or a pharmaceutically acceptable salt thereof, or a pharmaceutically acceptable solvate of either entity, together with a pharmaceutically acceptable diluent or carrier.

It further provides a veterinary formulation comprising an aryl sulfonamide derivatized Stat3 inhibitor provided herein, or a veterinarily acceptable salt thereof, or a veterinarily acceptable solvate of either entity, together with a veterinarily acceptable diluent or carrier.

The invention also provides an aryl sulfonamide derivatized Stat3 inhibitor provided herein, or a pharmaceutically acceptable salt thereof, or a pharmaceutically acceptable solvate of either entity, or a pharmaceutical composition containing any of the foregoing, for use as a human medicament.

In addition, it provides an aryl sulfonamide derivatized Stat3 inhibitor compound provided herein, or a veterinarily acceptable salt thereof, or a veterinarily acceptable solvate of either entity, or a veterinary formulation containing any of the foregoing, for use as an animal medicament.

In yet another aspect, the invention provides the use of an aryl sulfonamide derivatized Stat3 inhibitor compound provided herein, or a pharmaceutically acceptable salt thereof, or a pharmaceutically acceptable solvate of either entity, for the manufacture of a human medicament for the curative or prophylactic treatment of a medical condition for which a Stat3 inhibitor is indicated.

It also provides the use of an aryl sulfonamide derivatized Stat3 inhibitor compound provided herein, or a veterinarily acceptable salt thereof, or a veterinarily acceptable solvate of either entity, for the manufacture of an animal medicament for the curative or prophylactic treatment of a medical condition for which a Stat3 inhibitor is indicated.

Moreover, the invention includes use of the compounds and compositions provided herein for methods for treating and/or preventing, in whole or in part, various diseases, disorders and conditions, including but not limited to hyperproliferative disease such as cancer.

The invention also includes pharmaceutical compositions, including tablets and capsules and other oral delivery forms and formulations, comprising a pharmaceutically acceptable carrier and therapeutically effective amounts of an aryl sulfonamide derivatized Stat3 inhibitor as provided herein.

The invention includes methods for the use of therapeutically effective amounts of a Stat3 inhibitor provided herein in the manufacture of a medicament. Such medicaments include, for example, tablets, capsules, solutions and suspensions for parenteral and oral delivery forms and formulations. Such medicaments include those for the treatment of a subject as disclosed herein.

The compounds of the invention, particularly aryl sulfonamide derivatized Stat3 inhibitor salts, and hydrates, for example, in the disclosed crystal form, may also be prepared with another anti-cancer agent.

Doses for such aryl sulfonamide derivatized Stat3 inhibitors, salts and/or solvates as provided herein are envisaged to be administered in a therapeutically effective amount, for example, to inhibit cancer, delay tumor progression, and/or to reduce multidrug resistance in a subject.

The invention includes a formulation comprising an aryl sulfonamide derivatized Stat3 inhibitor provided herein in amounts effective to reduce glutathione transport in the body of a subject. Such formulations include, for example, tablets, capsules, solutions and suspensions for parenteral and oral delivery forms and formulations.

### Methods of Administration of Stat3 Inhibitors

The present invention is based a surprising, and unexpected, discovery that the aryl sulfonamide derivatized Stat3 inhibitors of this invention are potent, selective inhibitors of Stat3 with anti-tumor activity. In addition, aspects of the present invention are based on the surprising discovery that the potent and selective Stat3 inhibitors of this invention have the ability to treat cancer, for example, to suppress, and/or prevent metastasis of cancer cells.

For the purpose of the current disclosure, the following definitions shall, in their entireties, be used to define technical terms, and to define the scope of the composition of matter for which protection is sought in the claims.

The instant disclosure provides methods of treatment by administration to a subject of one or more effective dose(s) of aryl sulfonamide derivatized Stat3 inhibitors for a duration to achieve the desired therapeutic effect. The subject is preferably a mammal, including, but not limited to, animals such as cows, pigs, horses, chickens, cats, dogs, etc., and is most preferably human.

In some embodiments, compositions comprising aryl sulfonamide derivatized Stat3 inhibitor compounds of the present invention are delivered in accordance with the methods of the invention, e.g., encapsulation in liposomes, microparticles or microcapsules. Methods of introduction include, but are not limited to, topical, subcutaneous, intradermal, intramuscular, intraperitoneal, intravenous, subcutaneous, intranasal, epidural, and oral routes. For treatment of certain cancers, topical, subcutaneous, intradermal, and systemic deliveries can be particularly efficacious.

In some embodiments, aryl sulfonamide derivatized Stat3 inhibitors are administered by any convenient route, for example by infusion or bolus injection, by absorption through epithelial or mucocutaneous linings (e.g., oral mucosa, rectal and intestinal mucosa). In some embodiments, the Stat3 inhibitors are administered together with other biologically active agents. In some embodiments, administration is systemic or local. In some embodiments, pharmaceutical compositions comprising a Stat3 inhibitor are introduced into the central nervous system by any suitable route, including intraventricular and intrathecal injection; intraventricular injection may be facilitated by an intraventricular catheter, for example, attached to a reservoir, such as an Ommaya reservoir. In some embodiments, pulmonary administration is employed, e.g., by use of an inhaler or nebulizer, and formulation with an aerosolizing agent. In some embodiments, pharmaceutical compositions comprising Stat3 inhibitor are administered locally to the area in need of treatment by topical application, by injection, by means of a catheter, by means of a suppository, or by means of an implant, said implant being of a porous, non-porous, or gelatinous material, including membranes, such as Silastic™ membranes, or fibers.

Still other modes of administration of aryl sulfonamide derivatized Stat3 inhibitors involve delivery in a controlled release system. In some embodiments, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In some embodiments, polymeric materials can be used, or a controlled release system is placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

### Forms and Dosages of Compositions Comprising Stat3 Inhibitors

As used herein, for cancer treatment, lyophilized formulation and liquid formulation suitable for injection are particularly efficacious. Suitable dosage forms of Stat3 inhibitors for use in embodiments of the present invention encompass physiologically/pharmaceutically acceptable carriers that are inherently non-toxic and non-therapeutic. Examples of such carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, P6N (Neumedicines, Pasadena, Ca.) and PEG. Carriers for topical or gel-based forms of Stat3 inhibitors include polysaccharides, such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Suitable examples of sustained-release preparations include semipermeable matrices of solid hydrophobic polymers containing the polypeptide, which matrices are in the form of shaped articles, e.g., films, or microcapsules. Examples of sustained-release matrices include polyesters, hydrogels (for example, poly(2-hydroxyethyl-methacrylate) as described by Langer et al., supra and Langer, supra, or poly(vinylalcohol), polylactides (U.S. Pat. No. 3,773,919, herein incorporated by reference), copolymers of L-glutamic acid and gamma-ethyl-L-glutamate (Sidman et al, supra), non-degradable ethylene-vinyl acetate (Langer et al., supra), degradable lactic acid-glycolic acid copolymers such as the Lupron Depot™ (injectable microspheres composed of lactic acid-glycolicacid copolymer and leuprolide acetate), and poly-D-(−)-3-hydroxybutyric acid. While polymers, such as ethylene-vinyl acetate and lactic acid-glycolic acid, enable release of molecules for over 100 days, certain hydrogels release proteins for shorter time periods. When encapsulated Stat3 inhibitors remain in the body for a long time, they may denature, or aggregate, as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

In the case of administrations over several days or longer, depending on the condition, the treatment is sustained until a desired suppression of disease symptoms occurs. The progress of this therapy is monitored by conventional techniques and assays.

Therapeutic formulations comprising aryl sulfonamide derivatized Stat3 inhibitors are prepared for storage by mixing Stat3 inhibitors, having the desired degree of purity, with optional physiologically acceptable carriers, excipients, or stabilizers (Remington's Pharmaceutical Sciences, 16th edition, Osol, A., Ed., (1980)), in the form of lyophilized cake, or aqueous solutions. Acceptable carriers, excipients, or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or non-ionic surfactants such as Tween®, Pluronics™ or polyethylene glycol (PEG).

The term “buffer,” as used herein, denotes a pharmaceutically acceptable excipient, which stabilizes the pH of a pharmaceutical preparation. Pharmaceutically acceptable buffers include, but are not limited to, histidine-buffers, citrate-buffers, succinate-buffers, acetate-buffers, phosphate-buffers, arginine-buffers, or mixtures thereof. The abovementioned buffers are generally used in an amount of about 1 mM to about 100 mM, of about 5 mM to about 50 mM and of about 10-20 mM. In some embodiments, the pH of the buffered solution is at least 4.0, at least 4.5, at least 5.0, at least 5.5 or at least 6.0. In some embodiments, the pH of the buffered solution is less than 7.5, less than 7.0, or less than 6.5. In some embodiments, the pH of the buffered solution is about 4.0 to about 7.5, about 5.5 to about 7.5, about 5.0 to about 6.5, and about 5.5 to about 6.5 with an acid or a base described herein, e.g. hydrochloric acid, acetic acid, phosphoric acid, sulfuric acid and citric acid, sodium hydroxide and potassium hydroxide. As used herein when describing pH, “about” means plus or minus 0.2 pH units.

As used herein, the term “surfactant” can include a pharmaceutically acceptable excipient which is used to protect protein formulations against mechanical stresses, like agitation and shearing. Examples of pharmaceutically acceptable surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Suitable surfactants include polyoxyethylenesorbitan-fatty acid esters such as polysorbate 20, (sold under the trademark Tween 20®) and polysorbate 80 (sold under the trademark Tween 80®). Suitable polyethylene-polypropylene copolymers are those sold under the names Pluronic® F68 or Poloxamer 188®. Suitable Polyoxyethylene alkyl ethers are those sold under the trademark Brij®. Suitable alkylphenolpolyoxyethylene esthers are sold under the tradename Triton-X. When polysorbate 20 (Tween 20®) and polysorbate 80 (Tween 80®) are used, they are generally used in a concentration range of about 0.001 to about 1%, of about 0.005 to about 0.2% and of about 0.01% to about 0.1% w/v (weight/volume).

As used herein, the term “stabilizer” can include a pharmaceutically acceptable excipient, which protects the active pharmaceutical ingredient and/or the formulation from chemical and/or physical degradation during manufacturing, storage and application. Stabilizers include, but are not limited to, sugars, amino acids, polyols, cyclodextrins (e.g. hydroxypropyl-beta-cyclodextrine, sulfobutylethyl-beta-cyclodextrin, beta-cyclodextrin), polyethylenglycols (e.g. PEG 3000, PEG 3350, PEG 4000, PEG 6000), albumin, human serum albumin (HSA), bovine serum albumin (BSA), salts (e.g., sodium chloride, magnesium chloride, calcium chloride), chelators (e.g., EDTA) as hereafter defined. In some embodiments, stabilizers are present in the formulation in an amount of about 10 to about 500 mM, an amount of about 10 to about 300 mM, or in an amount of about 100 mM to about 300 mM. In some embodiments, exemplary Stat3 inhibitors are dissolved in an appropriate pharmaceutical formulation, wherein it is stable.

In some embodiments, aryl sulfonamide derivatized Stat3 inhibitors are entrapped in microcapsules prepared by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are disclosed in Remington's Pharmaceutical Sciences, supra.

Aryl sulfonamide derivatized Stat3 inhibitors to be used for in vivo administration must be sterile. This is readily accomplished by filtration through sterile filtration membranes, prior to, or following, lyophilization and reconstitution. Stat3 inhibitors ordinarily will be stored in lyophilized form, or in solution. Therapeutic Stat3 inhibitors compositions generally are placed into a container having a sterile access port, for example, an intravenous solution bag, or vial, having a stopper pierceable by a hypodermic injection needle.

When applied topically, aryl sulfonamide derivatized Stat3 inhibitors is suitably combined with other ingredients, such as carriers and/or adjuvants. There are no limitations on the nature of such other ingredients, except that they must be physiologically acceptable and efficacious for their intended administration, and cannot degrade the activity of the active ingredients of the composition. Examples of suitable vehicles include ointments, creams, gels, or suspensions, with, or without, purified collagen. In some embodiments, the compositions are impregnated into articles which can include or exclude transdermal patches, plasters, and bandages, preferably in liquid or semi-liquid form.

In some embodiments, a gel formulation of aryl sulfonamide derivatized Stat3 inhibitor compound is formulated in a liquid composition by mixing the compound with an effective amount of a water-soluble polysaccharide, or synthetic polymer, such as PEG, to form a gel of the proper viscosity to be applied topically. In some embodiments, the polysaccharide can include or exclude cellulose derivatives, such as etherified cellulose derivatives, including alkyl celluloses, hydroxyalkyl celluloses, and alkylhydroxyalkyl celluloses (e.g., methylcellulose, hydroxyethyl cellulose, carboxymethyl cellulose, hydroxypropyl methylcellulose, and hydroxypropyl cellulose); starch and fractionated starch; agar; alginic acid and alginates; gum arabic; pullullan; agarose; carrageenan; dextrans; dextrins; fructans; inulin; mannans; xylans; arabinans; chitosans; glycogens; glucans; and synthetic biopolymers; as well as gums such as xanthan gum; guar gum; locust bean gum; gum arabic; tragacanth gum; and karaya gum; and derivatives and mixtures thereof. The preferred gelling agent herein is one that is inert to biological systems, nontoxic, simple to prepare, and not too runny or viscous, and will not destabilize the Stat3 inhibitor molecule held within it.

Preferably the polysaccharide is an etherified cellulose derivative, more preferably one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. Most preferred herein is methylcellulose.

In some embodiments, the polyethylene glycol useful for gelling is a mixture of low and high molecular weight PEGs to obtain the proper viscosity. For example, a mixture of a PEG of molecular weight 400-600 with one of molecular weight 1500 would be effective for this purpose, when mixed in the proper ratio to obtain a paste.

The term “water soluble,” as applied to the polysaccharides and PEGs, is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. In some embodiments, the cellulose derivatives are in the form of alkali metal salts, for example, the Li, Na, K, or Cs salts.

An effective amount of aryl sulfonamide derivatized Stat3 inhibitors to be employed therapeutically will depend, for example, upon the therapeutic objectives, the route of administration, and the condition of the patient. Accordingly, it will be necessary for the therapist to titer the dosage and modify the route of administration, as required to obtain the optimal therapeutic effect. Typically, the clinician will administer Stat3 inhibitors until a dosage is reached that achieves the desired effect. In certain embodiments, the appropriate dosing is determined based on an amount of Stat3 inhibitors administered per surface area of the affected region.

“Near the time of administration of the treatment” refers to the administration of Stat3 inhibitors at any reasonable time period, either before, and/or after the administration of the treatment, such as about one month, about three weeks, about two weeks, about one week, several days, about 120 hours, about 96 hours, about 72 hours, about 48 hours, about 24 hours, about 20 hours, several hours, about one hour or minutes. Near the time of administration of the treatment may also refer to either the simultaneous, or near simultaneous, administration of the treatment and aryl sulfonamide derivatized Stat3 inhibitors, i.e., within minutes to one day.

“Chemotherapy” refers to any therapy that includes natural or synthetic agents now known, or to be developed in the medical arts. Examples of chemotherapy include the numerous cancer drugs that are currently available. However, chemotherapy also includes any drug, natural or synthetic, that is intended to treat a disease state. In certain embodiments of the invention, chemotherapy may include the administration of several state of the art drugs intended to treat the disease state. Examples include combined chemotherapy with docetaxel, cisplatin, and 5-fluorouracil, for patients with locally advanced squamous cell carcinoma of the head (Tsukuda, M. et al., Int J Clin Oncol. 2004 June; 9 (3): 161-6), and fludarabine and bendamustine in refractory and relapsed indolent lymphoma (Konigsmann M, et al., Leuk Lymphoma. 2004; 45 (9): 1821-1827).

As used herein, exemplary sources of therapeutic or accidental ionizing radiation can include, for example, alpha, beta, gamma, x-ray, and neutron sources.

“Radiation therapy” refers to any therapy where any form of radiation is used to treat the disease state. The instruments that produce the radiation for the radiation therapy are either those instruments currently available, or to be available in the future.

“Chemoprotection or radioprotection” refers to protection from, or an apparent decrease in, the associated hematopoietic toxicity of a treatment intended to target the disease state.

“Solid tumors” generally refers to the presence of cancer of body tissues other than blood, bone marrow, or the lymphatic system. The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

### EXAMPLES

The invention is now described with reference to the following Examples. These Examples are provided for the purpose of illustration only, and the invention is not limited to these Examples, but rather encompasses all variations that are evident as a result of the teaching provided herein.

The Examples described herein demonstrate that the potent and selective aryl sulfonamide derivatized Stat3 inhibitors of Formulae I-V, including compounds of Examples 1-88 have efficacy for treating cancer and other proliferative diseases. Aspects and embodiments of the instant disclosure stem from the unexpected discovery that certain Stat3 inhibitor formulations have surprising, and unexpected, utility and efficacy when administered to a subject.

The therapeutically effective aryl sulfonamide derivatized Stat3 inhibitors of this invention are prepared according to the synthetic scheme outlined above. However, the invention is not limited to those methods. The compositions may also be prepared as described herein for structurally related compounds.

### Synthesis of Selected Compounds of the Present Invention.

General Methods for Chemistry. All reagents and solvents were purchased from commercial sources and used without further purification. All moisture sensitive reactions were performed under a static atmosphere of nitrogen or argon in oven dried glassware. Tetrahydrofuran (THF), dichloromethane (DCM), diethyl ether (Et₂O), toluene, dimethylformamide (DMF) used in the reactions were dried by being passed through a SPS system. Other anhydrous solvents were purchased from commercial sources. Thin layer chromatography (TLC) was performed on glass plates, 250-1000 μm. Flash column chromatography was performed on silica gel, 200-400 mesh. ¹H NMR spectra were obtained as CDCl₃, CD₃OD, or (CD₃)₂SO, solutions using an Agilent 300 MHz NMR spectrometer with a Agilent DD2 console, and chemical shifts were expressed in δ (ppm) using residual solvent (CDCl₃, 7.26 ppm; CD₃OD, 3.31 ppm; and (CD₃)₂SO, 2.50 ppm) as the reference standard. When peak multiplicities are reported, the following abbreviations are used: s (singlet), d (doublet), t (triplet), q (quartet), m (multiplet), br-s (broadened singlet), dd (doublet of doublets), dt (doublet of triplets). Coupling constants, when reported, are reported in hertz (Hz). All compounds were analyzed by LC/MS (liquid chromatography/mass spectrometry) using an Agilent Triple Quad 640 LC/MS. Ionization was generally achieved via electron spray (ESI) unless otherwise indicated. The LC fraction detection consisted of a variable wavelength detector and all tested compounds had purity greater than 95%. High resolution mass spectral (HRMS) data was obtained for all tested compounds using either and Agilent 6200 LC/MSD TOF or an Agilent 6545 Q-TOF LC/MS and reported exact masses were calculated based on an algorithm using MS (ESI) m/z for [M+H]⁺ and [M+Na]⁺ adducts and were within 5 ppm of the expected target mass. Chiral molecules were analyzed by chiral HPLC using Chiralpak AD-H or OD-H columns (4.6 mm×250 mm, UV detection at 254 or 261 nm), eluents used were hexane and i-PrOH.

### Commonly Used Abbreviations

Commonly used abbreviations include: acetic acid (AcOH), acetonitrile (MeCN, CH₃CN), azobisisobutyronitrile (AIBN), 2,2′-bis(diphenylphosphino)-1,1′-binaphthyl (BINAP), 4,5-Bis(diphenylphosphino)-9,9-dimethylxanthene (Xantphos), tert-butoxycarbonyl (Boc), benzyl (Bn), butyl (Bu), benzyloxycarbonyl (CBZ or Z), ceric ammonium nitrate (CAN), cyclohexyl (Cy), cyclopentyl (Cp), dibenzylideneacetone (dba), dichloroethane (DCE), dichloromethane (DCM), N-[(Dimethylamino)-1H-1,2,3-triazolo-[4,5-b]pyridin-1-ylmethylene]-N-methylmethanaminium hexafluorophosphate N-oxide (HATU), 2-Dicyclohexylphosphino-2′,6′-dimethoxybiphenyl (SPhos), diphenylphosphoryl azide (DPPA),di-iso-propylethylamine (DIPEA), methanesulfonyl chloride (MsCl), 4-N,N-dimethylaminopyridine (DMAP), N,N-dimethylformamide (DMF), dimethyl sulfoxide (DMSO), electrospray ionization (ESI), ethyl (Et), ethyl acetate (EtOAc, EA), 1-Ethyl-3-(3-dimethylaminopropyl)carbodiimide (EDCI), ethanol (EtOH), diethyl ether (Et₂O, ether), 9H-fluoren-9-yl)methoxy)carbonyl (Fmoc), high resolution mass spectrometry (HRMS), high pressure liquid chromatography (HPLC), lithium hexamethyldisilazane (LiHMDS), liquid chromatography-mass spectrometry (LCMS), methanol (MeOH), melting point (mp or MP), methyl (Me), mass spectrum (ms or MS), methylmagnesium bromide (MeMgBr), N-bromosuccinumude (NB S), N-methylmorpholine (NMM), N-methylpyrrolidone (NMP), palladium on carbon (Pd/C), phenyl (Ph), potassium hexamethyldisilazane (KHMDS), propyl (Pr), iso-propyl (i-Pr), room temperature (rt or RT), sodium hexamethyldisilazane (NaHMDS), triethylamine (TEA, or Et₃N), 2-(trimethylsilyl)ethoxymethyl (SEM), trifluoroacetic acid (TFA), trifluoroacetic anhydride (TFAA), thin layer chromatography (TLC), and tetrahydrofuran (THF), 2-methyltetrahydrofuan (MeTHF), (STAT) signal transducer and activator of transcription; (EMSA) electrophoretic mobility shift assay; (MAPK) mitogen-activated protein kinase; (ERK) extracellular signal-regulated kinases.

### Example 1

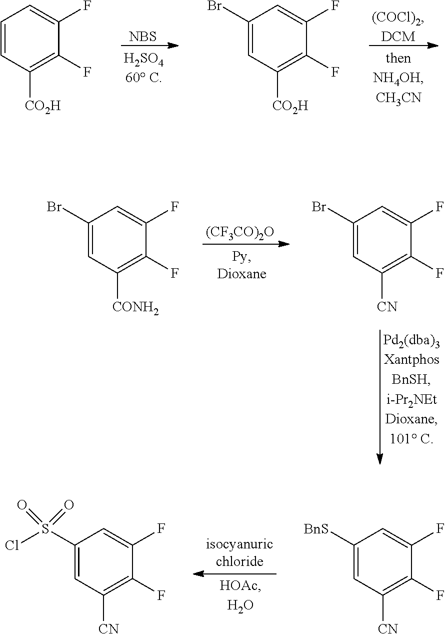

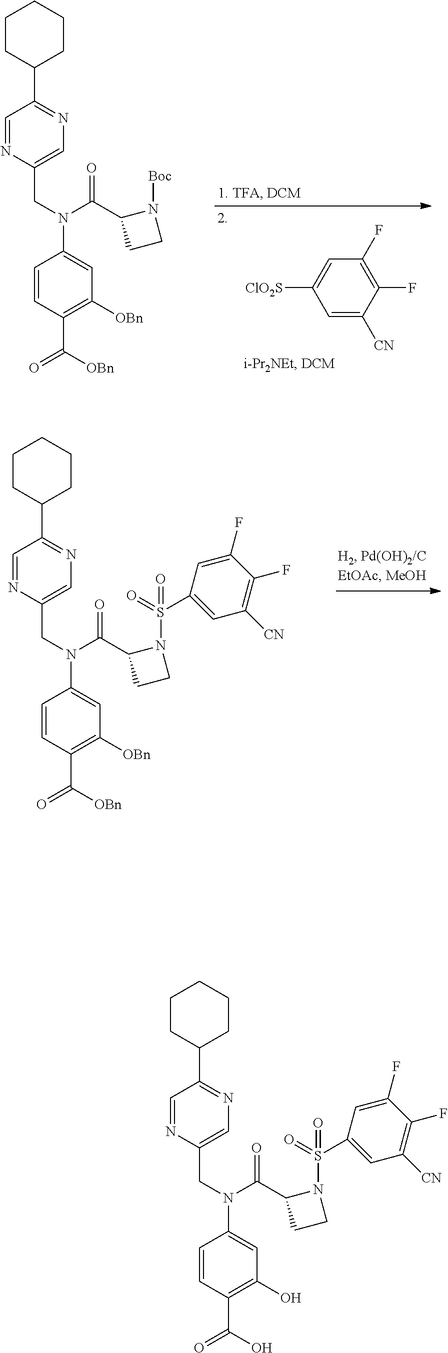 [SMILES: CC(C)(C)OC(=O)N1CC[C@@H]1C(=O)N(Cc1cnc(C2CCCCC2)cn1)c1ccc(C(=O)OCc2ccccc2)c(OCc2ccccc2)c1 ; N#Cc1cc(S(=O)(=O)Cl)cc(F)c1F ; N#Cc1cc(S(=O)(=O)N2CC[C@@H]2C(=O)N(Cc2cnc(C3CCCCC3)cn2)c2ccc(C(=O)OCc3ccccc3)c(OCc3ccccc3)c2)cc(F)c1F ; N#Cc1cc(S(=O)(=O)N2CC[C@@H]2C(=O)N(Cc2cnc(C3CCCCC3)cn2)c2ccc(C(=O)O)c(O)c2)cc(F)c1F]

### (R)-4-(1-((3-cyano-4,5-difluorophenyl)sulfonyl)-N-((5-cyclohexylpyrazin-2-yl)methyl)azetidine-2-carboxamido)-2-hydroxybenzoic acid

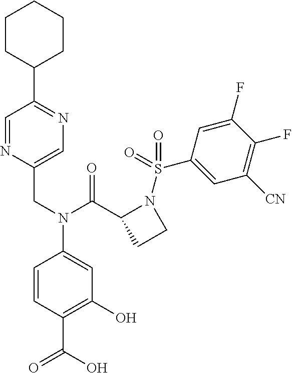 [SMILES: N#Cc1cc(S(=O)(=O)N2CC[C@@H]2C(=O)N(Cc2cnc(C3CCCCC3)cn2)c2ccc(C(=O)O)c(O)c2)cc(F)c1F]

Step 1. To 2,3-difluorobenzoic acid (7.0 g) was added concentrated H2SO4 (35.9 ml) and N-bromosuccinimide (8.26 g). The reaction mixture was heated with stirring at sixty degrees Celsius for three hours under argon. Reaction was then allowed to cool to room temperature and poured onto ice water. This mixture was allowed to stir at room temperature for five minutes, and then filtered. The solid was then washed with room temperature water. The solid was then dissolved in ethyl acetate and extracted with 3M sodium hydroxide (×2). The ethyl acetate layer can then be discarded, and the aqueous layer was then acidified with 3M HCl. The aqueous layer was extracted with ethyl acetate (×2). The combined extracts were washed with water, brine, dried over Na2SO4 and concentrated to obtain 5-bromo-2,3-difluorobenzoic acid (8.6 g, 82% yield). 1H NMR (300 MHz, CDCl3) δ 7.96 (ddd, J=5.5, 2.6, 2.1 1H), 7.61 (ddd, J=9.0, 6.4, 2.6 1H). 19F NMR (282 MHz, CDCl3) δ−132.54 (ddd, J=20.1, 9.0, 2.1 1F), −134.56 (ddd, J=20.1, 6.41, 5.5 1F).

Step 2. To a solution of 5-bromo-2,3-difluorobenzoic acid (7.8 g) in dichloromethane (60 ml) was added oxalyl chloride (4.25 ml) followed by dry DMF (4-6 drops) under argon. The mixture was allowed to stir at room temperature for 2.5 hours and then concentrated. Dry acetonitrile (30 ml) was added, and the solution was poured onto cold concentrated ammonium hydroxide (318 ml). The mixture was allowed to reach room temperature and then stirred for 15 minutes. Water was added to the mixture and then it was extracted with ethyl acetate (×2). The extracts were then washed with water, brine, dried over Na2SO4 and concentrated to obtain 5-bromo-2,3-difluorobenzamide (7.5 g, 97% yield). 1H NMR (300 MHz, CDCl3) δ 8.04 (ddd, J=5.8, 2.5, 2.1 1H), 7.53 (ddd, J=8.9, 6.9, 2.5 1H). 19F NMR (282 MHz, CDCl3) δ−133.98 (ddd, J=21.9, 8.9, 2.1 1F) −139.98 (m, 1F).

Step 3. To a solution of 5-bromo-2,3-difluorobenzamide (6.84 g) in dioxane (45 ml) was added anhydrous pyridine (4.71 ml). The solution was cooled on an ice-water bath, and trifluoroacetic anhydride (4.47 ml) was added. The reaction was allowed to reach room temperature and then stirred for four and a half hours. The mixture was poured onto water and extracted with ethyl acetate (×2). The organic extracts were then washed with sodium bicarbonate (×2). The combined organic extracts were then washed with water, brine, dried, and concentrated to obtain 5-bromo-2,3-difluorobenzonitrile (5.7 g, 90% yield) 1H NMR (300 MHz, CDCl3) δ 7.64 (ddd, J=9.1, 6.8, 2.3 1H) 7.58 (ddd, J=4.7, 2.3, 1.9 1H). 19F NMR (300 MHz, CDCl3) δ−130.54 (ddd, J=20.1, 9.1, 1.9 1F), −131.25 (ddd, J=20.1, 6.8, 4.7 1F).

Step 4. To 5-bromo-2,3-difluorobenzonitrile (9.0 g) was added Pd2(dba)3 (0.94 g) and Xantphos (1.18 g). The reaction vessel was then flushed with argon. To the solids was added dioxane (100 ml), followed by i-Pr2NEt (14.1 ml), and benzyl mercaptan (5 ml). The reaction mixture was allowed to stir at 101 degrees celsius for 19 hours. After cooling to room temperature, water was added, and the mixture was then extracted with ethyl acetate (×2). The combined organic extracts were washed with water, brine, dried and concentrated. Purification by column chromatography (1:3 hexane/toluene) gave 2,3-difluoro-5-(phenylthio)benzonitrile (65% Yield) 1H NMR (300 MHz, CDCl3) δ 7.3 (m, 2H), 4.13 (s, 2H). 19F NMR (282 MHz, CDCl3) δ−132.8 (ddd, J=20.4, 9.9, 1.4 1F), −132.97 (ddd, J=20.4, 7.4, 4.5 1F)

Step 5. To a solution of 2,3-difluoro-5-(phenylthio)benzonitrile (3.87 g) in HPLC acetonitrile (80 ml) was added acetic acid (3.87 ml) and HPLC water (1.93 ml). The mixture was cooled to zero degrees Celsius and isocyanuric chloride was added (6.88 g). The ice bath was removed and the reaction was stirred for one hour. Added water to the reaction and extracted with ethyl acetate (×2). The organic extracts were washed with pH 7 buffer, water, brine, dried, and concentrated. Purification by column chromatography (96:4 hexanes-ethyl acetate) gave 3-cyano-4,5-difluorobenzenesulfonyl chloride (2.5 g, 70% yield). 1H NMR (300 MHz, CDCl3) δ 8.17 (m, 2H) 19F NMR (282 MHz, CDCl3) δ−116.8 (ddd, J=19.7, 6.51, 4.7 1F), −126.29 (ddd, J=19.7, 8.9, 2.6 1F)

Step 6. tert-butyl (R)-2-((3-(benzyloxy)-4-((benzyloxy)carbonyl)phenyl)((5-cyclohexylpyrazin-2-yl)methyl)carbamoyl)azetidine-1-carboxylate (64.9 mg, 0.094 mmol) was dissolved in a mixed solvent [DCM (0.9 mL) and TFA (0.3 mL)] at room temperature. After stirring for 1 h, the solvent was removed in vacuo, and then water and EtOAc were added to the residue. The crude products were extracted with EtOAc (×3), and the combined organic extracts were washed with brine, dried (MgSO4), and concentrated in vacuo to obtain the crude benzyl (R)-2-(benzyloxy)-4-(N-((5-cyclohexylpyrazin-2-yl)methyl)azetidine-2-carboxamido)benzoate (61 mg).

Step 7. Crude benzyl (R)-2-(benzyloxy)-4-(N-((5-cyclohexylpyrazin-2-yl)methyl)azetidine-2-carboxamido)benzoate (61 mg) was dissolved in DCM (1.0 mL). After cooling to 0° C., N,N-Diisopropylethylamine (0.08 mL, 0.46 mmol) and 3-cyano-4,5-difluorobenzene-1-sulfonyl chloride (48.2 mg, 0.203 mmol) in DCM (1.0 mL) were added. After stirring for 1 h at 0° C., the reaction mixture was quenched by adding saturated NaHCO3 solution. The crude products were extracted with DCM (×3), and the combined organic extracts were washed with brine, dried (MgSO4), and concentrated in vacuo. The residue was purified by flash column chromatography (hexane/EtOAc=5/1 to 2/1) to afford benzyl (R)-2-(benzyloxy)-4-(1-((3-cyano-4,5-difluorophenyl)sulfonyl)-N-((5-cyclohexylpyrazin-2-yl)methyl)azetidine-2-carboxamido)benzoate. 1H NMR (300 MHz, CDCl3) δ 8.48 (s, 1H), 8.39 (s, 1H), 8.13-8.19 (m, 1H), 8.05-8.08 (m, 1H), 7.86 (d, J=8.1 Hz, 1H), 7.32-7.44 (m, 10H), 6.93 (d, J=1.8 Hz, 1H), 6.85 (dd, J=1.8, 8.1 Hz, 1H), 5.38 (s, 2H), 5.20 (d, J=12.3 Hz, 1H), 5.03-5.13 (m, 2H), 4.89-4.96 (m, 2H), 3.90-3.98 (m, 1H), 3.53-3.59 (m, 1H), 2.72-2.82 (m, 1H), 2.04-2.14 (m, 1H), 1.85-1.99 (m, 5H), 1.75-1.82 (m, 1H), 1.26-1.64 (m, 5H).

Step 8. To a stirred solution of benzyl (R)-2-(benzyloxy)-4-(1-((3-cyano-4,5-difluorophenyl)sulfonyl)-N-((5-cyclohexylpyrazin-2-yl)methyl)azetidine-2-carboxamido)benzoate (55.5 mg, 0.077 mmol) in THF (6 mL) and methanol (6 mL) under nitrogen was added 20% Pd(OH)2 on carbon (8 mg). The solution was placed under a hydrogen balloon and stirred for 16 hour. Additional 20% Pd(OH)2 on carbon (8 mg) was added and stirred 10 h. The solution was filtered through Celite®, washed with methanol and evaporated under reduced pressure. Purification by preparative thin layer chromatography (ethyl acetate/hexane/methanol 4:4:1) gave (R)-4-(1-((3-cyano-4,5-difluorophenyl)sulfonyl)-N-((5-cyclohexylpyrazin-2-yl)methyl)azetidine-2-carboxamido)-2-hydroxybenzoic acid (17% yield) as a white foam. 1H NMR (300 MHz, CDCl3) δ 11.20 (brs, 1H, OH), 8.95 (s, 1H), 8.46 (s, 1H), 8.13-8.19 (m, 1H), 8.06-8.08 (m, 1H), 7.74 (d, J=8.4 Hz, 1H), 6.70 (d, J=8.4 Hz, 1H), 6.65 (s, 1H), 5.20 (d, J=14.7 Hz, 1H), 4.95-5.03 (m, 2H), 3.95-4.03 (m, 1H), 3.63-3.70 (m, 1H), 2.82-2.93 (m, 1H), 2.26-2.37 (m, 1H), 1.26-2.07 (m, 11H).

### Examples 2 and 3

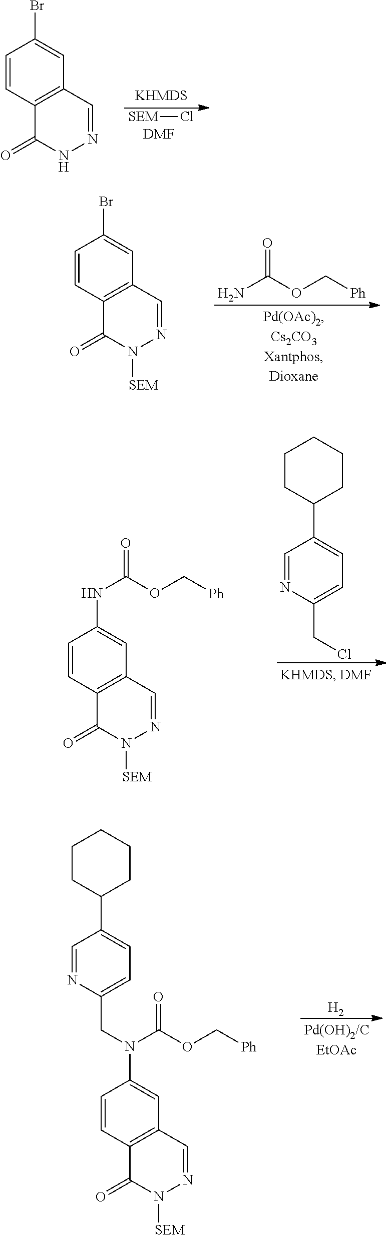

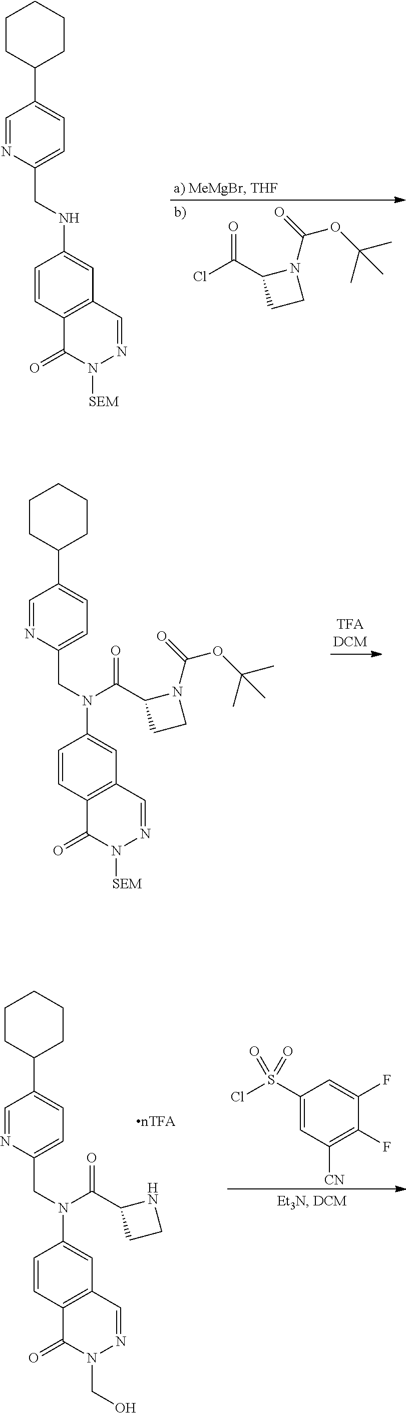

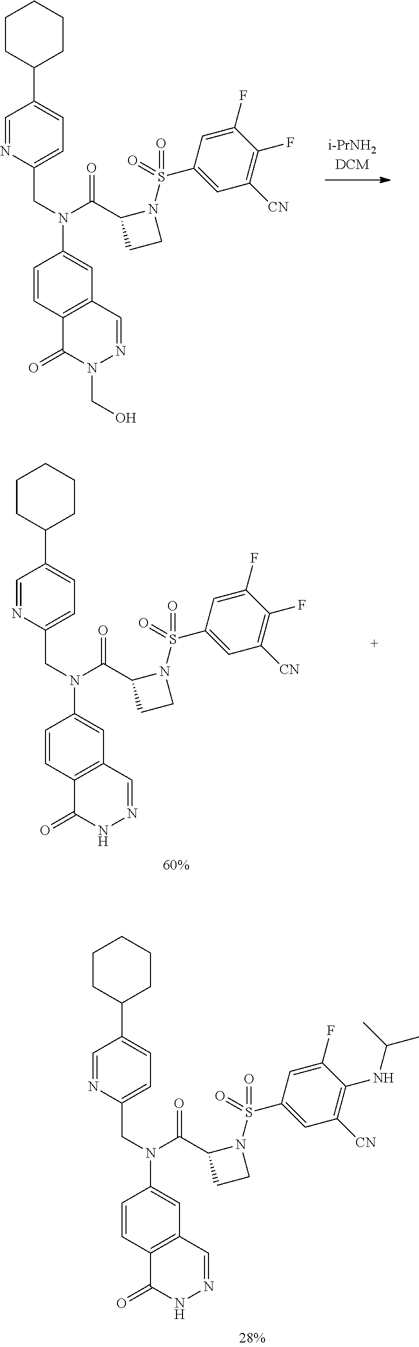 [SMILES: N#Cc1cc(S(=O)(=O)N2CC[C@@H]2C(=O)N(Cc2ccc(C3CCCCC3)cn2)c2ccc3c(=O)n(CO)ncc3c2)cc(F)c1F ; N#Cc1cc(S(=O)(=O)N2CC[C@@H]2C(=O)N(Cc2ccc(C3CCCCC3)cn2)c2ccc3c(=O)[nH]ncc3c2)cc(F)c1F ; CC(C)Nc1c(F)cc(S(=O)(=O)N2CC[C@@H]2C(=O)N(Cc2ccc(C3CCCCC3)cn2)c2ccc3c(=O)[nH]ncc3c2)cc1C#N]

### Example 2

### (R)-1-((3-cyano-4,5-difluorophenyl)sulfonyl)-N-((5-cyclohexylpyridin-2-yl)methyl)-N-(1-oxo-1,2-dihydroisoquinolin-6-yl)azetidine-2-carboxamide

### Example 3

### (R)-1-((3-cyano-5-fluoro-4-(isopropylamino)phenyl)sulfonyl)-N-((5-cyclohexylpyridin-2-yl)methyl)-N-(1-oxo-1,2-dihydrophthalazin-6-yl)azetidine-2-carboxamide

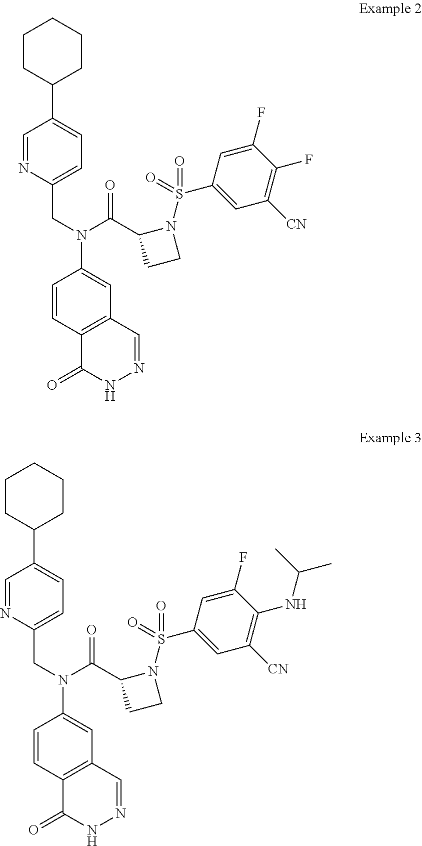 [SMILES: N#Cc1cc(S(=O)(=O)N2CC[C@@H]2C(=O)N(Cc2ccc(C3CCCCC3)cn2)c2ccc3c(=O)[nH]ncc3c2)cc(F)c1F ; CC(C)Nc1c(F)cc(S(=O)(=O)N2CC[C@@H]2C(=O)N(Cc2ccc(C3CCCCC3)cn2)c2ccc3c(=O)[nH]ncc3c2)cc1C#N]

Step 1. To a solution of 6-bromophthalazin-1(2H)-one (2.03 g, 9.02 mmol) in DMF (40 mL) was added at 0 degrees C. KHMDS (1M in THF, 10.8 mL, 10.8 mmol) under argon. After 10 minutes, SEM-C1 (1.92 mL, 10.8 mmol) was added under argon. The mixture was allowed to reach room temperature and stirred for 26 h. Cold saturated ammonium chloride was added. The mixture was extracted with ethyl acetate (3×). The mixture was washed with water (2×), brine, dried (Na2SO4) and concentrated to dryness to obtain 6-bromo-2-((2-(trimethylsilyl)ethoxy)methyl)phthalazin-1(2H)-one (3.15 g), which was used as crude for next step. 1H NMR (300 MHz, Chloroform-d) δ 8.37-8.29 (m, 1H), 8.12 (s, 1H), 7.93-7.85 (m, 2H), 5.57 (s, 2H), 3.84-3.62 (m, 1H), 1.10-0.89 (m, 1H), 0.01 (s, 9H).

Step 2. A mixture of crude 6-bromo-2-((2-(trimethylsilyl)ethoxy)methyl)phthalazin-1(2H)-one (2.93 g, 8.25 mmol), Xantphos (0.477 g, 0.825 mmol), benzyl carbamate (1.87 g, 12.39 mmol), palladium acetate (0.185 g, 0.825 mmol), and cesium carbonate (5.37 g, 16.48 mmol) was thoroughly flushed with argon. Dioxane (103 mL) was added under argon. The mixture was heated at 100 degrees C. (oil bath temperature) for 18 h. After cooling, the mixture was poured onto saturated ammonium chloride and filtered. The black solid was washed with ethyl acetate. The filtrate was extracted with ethyl acetate (2×). The extract was washed with brine, dried (Na2SO4) and concentrated. Purification by flash column chromatography (8:2 to 7:3 hexane/ethyl acetate) gave benzyl (1-oxo-2-((2-(trimethylsilyl)ethoxy)methyl)-1,2-dihydrophthalazin-6-yl)carbamate (1.62 g, 46% for 2 steps) as an off-white solid.

Step 3. To a solution of benzyl (1-oxo-2-((2-(trimethylsilyl)ethoxy)methyl)-1,2-dihydrophthalazin-6-yl)carbamate (894 mg, 2.1 mmol) in DMF (11.8 mL) was added at 0 degrees C. KHMDS (1.0 M in THF, 2.72 mL, 2.72 mmol) under argon. After 5-10 minutes, 2-(chloromethyl)-5-cyclohexylpyridine (1.0 M in toluene, 2.72 mL, 2.72 mmol) was added at 0 degrees C. The mixture was allowed to reach room temperature and stirred for 20 h. Cold saturated ammonium chloride was added, and the mixture was extracted with ethyl acetate (2×). The extract was washed with water, brine, dried (Na2SO4) and concentrated. Purification by flash column chromatography (7:3 to 6:4 hexane/ethyl acetate) gave benzyl ((5-cyclohexylpyridin-2-yl)methyl)(1-oxo-2-((2-(trimethylsilyl)ethoxy)methyl)-1,2-dihydrophthalazin-6-yl)carbamate (859 mg, 68% yield). 1H NMR (300 MHz, Chloroform-d) δ 8.43 (d, J=2.3 Hz, 1H), 8.37 (dd, J=8.6, 0.8 Hz, 1H), 8.08 (s, 1H), 7.82-7.68 (m, 1H), 7.53-7.43 (m, 1H), 7.43-7.12 (m, 7H), 5.69-5.45 (m, 2H), 5.23 (d, J=0.8 Hz, 2H), 5.10 (s, 2H), 3.88-3.56 (m, 2H), 2.54 (s, 1H), 1.84 (dd, J=27.6, 10.8 Hz, 7H), 1.40 (q, J=10.6 Hz, 4H), 1.08-0.68 (m, 2H), 0.0 (s, 9H).

Step 4. To a solution of benzyl ((5-cyclohexylpyridin-2-yl)methyl)(1-oxo-2-((2-(trimethylsilyl)ethoxy)methyl)-1,2-dihydrophthalazin-6-yl)carbamate (850 mg, 1.42 mmol) in ethyl acetate (8.2 mL) and methanol (8.2 mL) was added 10% Pd(OH)2/C (82.3 mg). A balloon filled with hydrogen was set up, and the mixture was stirred for 16 h. The mixture was filtered through celite, and the filtrate was evaporated. Purification by flash chromatography column (6:4 to 45:55 hexane/ethyl acetate) gave 6-(((5-cyclohexylpyridin-2-yl)methyl)amino)-2-((2-(trimethylsilyl)ethoxy)methyl)phthalazin-1(2H)-one as a white solid (515 mg, 78% yield). 1H NMR (300 MHz, Chloroform-d) δ 8.47 (d, J=2.2 Hz, 1H), 8.23 (d, J=8.7 Hz, 1H), 8.01 (d, J=0.8 Hz, 1H), 7.60-7.50 (m, 1H), 7.32-7.22 (m, 1H), 7.09 (ddd, J=8.8, 2.4, 0.9 Hz, 1H), 6.67 (d, J=2.3 Hz, 1H), 5.75 (bs, 1H), 5.54 (s, 2H), 4.53 (d, J=4.9 Hz, 2H), 3.79-3.67 (m, 2H), 2.64-2.49 (m, 1H), 2.02-1.73 (m, 5H), 1.58-1.22 (m, 5H), 1.06-0.86 (m, 2H), 0.00 (s, 9H).

Step 5a. Acid chloride preparation: To a solution of (R)-1-(tert-butoxycarbonyl)azetidine-2-carboxylic acid (202.4 mg, 1.01 mmol) in dichloromethane (2.3 mL) was added oxalyl chloride (0.11 mL, 1.31 mmol), followed by DMF (1 drop) under argon. The mixture was concentrated in vacuo. Dichloroethane (2×) was added, and the mixture was evaporated back in vacuo. After drying at high vacuum for 15 minutes, the acid chloride was dissolved in THF (4 mL).

Step 5b. To a solution of 6-(((5-cyclohexylpyridin-2-yl)methyl)amino)-2-((2-(trimethylsilyl)ethoxy)methyl)phthalazin-1(2H)-one (234 mg, 0.503 mmol) in THF (4 mL) was added at 0 degrees C. under argon methylmagnesium bromide (1.4 M in THF, 0.902 mL, 1.27 mmol). The mixture was stirred for 10 minutes at 0 degrees C., and then the solution of the acid chloride in THF was added at 0 degrees C. The mixture was allowed to reach room temperature, and stirred for 1 h. Cold saturated ammonium chloride was added, and the mixture was extracted with ethyl acetate (2×). The extract was washed with brine, dried (Na2SO4) and concentrated. Purification by flash column chromatography (45:55 hexane/ethyl acetate, then added 2% methanol) gave tert-butyl (R)-2-(((5-cyclohexylpyridin-2-yl)methyl)(1-oxo-2-((2-(trimethylsilyl)ethoxy)methyl)-1,2-di hydrophthalazin-6-yl)carbamoyl)azetidine-1-carboxylate as a white foam (201 mg, 62% yield). 1H NMR (300 MHz, Chloroform-d) δ 8.44 (d, J=8.5 Hz, 1H), 8.34 (s, 1H), 8.12 (t, J=0.8 Hz, 1H), 7.84-7.37 (m, 4H), 5.58 (s, 2H), 5.20-4.97 (m, 2H), 4.71-4.51 (m, 1H), 4.11 (q, J=7.9 Hz, 1H), 3.82-3.68 (m, 3H), 2.60-2.45 (m, 1H), 2.32-2.05 (m, 2H), 1.95-1.70 (m, 5H), 1.52-1.12 (m, 14H), 1.07-0.94 (m, 2H), 0.02 (s, 9H).

Step 6. To a solution of tert-butyl (R)-2-(((5-cyclohexylpyridin-2-yl)methyl)(1-oxo-2-((2-(trimethylsilyl)ethoxy)methyl)-1,2-di hydrophthalazin-6-yl)carbamoyl)azetidine-1-carboxylate (197 mg, 0.304 mmol) in dichloromethane (2 mL) was added trifluoroacetic acid (1 mL) under argon. The mixture was stirred for 2 h, then concentrated to dryness. Dichloroethane (2×) was added and the mixture was concentrated back to dryness to obtain (R)—N-((5-cyclohexylpyridin-2-yl)methyl)-N-(2-(hydroxymethyl)-1-oxo-1,2-dihydrophthalazin-6-yl)azetidine-2-carboxamide TFA salt as a thick oil (260 mg), and used as such for the next reaction. 1H NMR (300 MHz, DMSO-d6) (mixture of 2-hydroxymethyl and 2-deshydroxymethyl) δ 12.82 (bs, 0.7H), 9.23-8.84 (m, 2H), 8.47-8.22 (m, 2H), 8.01 (s, 1H), 7.90 (d, J=8.1 Hz, 1H), 7.73 (d, J=8.4 Hz, 1H), 7.43 (d, J=8.1 Hz, 1H), 5.41 (s, 1.4H, corresponds contribution of 2-hydroxymethyl), 5.30-4.86 (m, 3H), 4.22-3.85 (m, 2H), 2.65-2.32 (m, 3H), 1.98-1.60 (m, 5H), 1.56-1.06 (m, 5H).

Step 7. To a solution of (R)—N-((5-cyclohexylpyridin-2-yl)methyl)-N-(2-(hydroxymethyl)-1-oxo-1,2-dihydrophthalazin-6-yl)azetidine-2-carboxamide TFA salt (220 mg) in dichloromethane (20 mL) was added triethylamine (0.143 mL, 1.03 mmol) followed by powder 3-cyano-4,5-difluorobenzenesulfonyl chloride (69.6 mg, 0.29 mmol) under argon. The mixture was stirred at room temperature for 2.5 h, then washed with water, dried (Na2SO4) and concentrated. Purification by flash column chromatography (4:6 hexane/ethyl acetate with 2% methanol to 3:7 hexane/ethyl acetate with 4% methanol) gave (R)-1-((3-cyano-4,5-difluorophenyl)sulfonyl)-N-((5-cyclohexylpyridin-2-yl)methyl)-N-(2-(hydroxymethyl)-1-oxo-1,2-dihydrophthalazin-6-yl)azetidine-2-carboxamide (58 mg, 45% for two reactions). 1H NMR (300 MHz, Chloroform-d) (mixture of 2-hydroxymethyl and 2-deshydroxymethyl) δ 11.56-11.33 (bs, 0.7H), 8.47-8.32 (m, 2H), 8.22-8.03 (m, 3H), 7.72-7.50 (m, 3H), 7.44-7.25 (m, 1H), 5.65 (s, 1.4H, corresponds to contribution of 2-hydroxymethyl), 5.17-4.95 (m, 3H), 4.12-3.88 (m, 1H), 3.77-3.51 (m, 1H), 2.65-2.26 (m, 3H), 1.99-1.70 (m, 5H), 1.53-1.29 (m, 5H).

Step 8. To a solution of (R)-1-((3-cyano-4,5-difluorophenyl)sulfonyl)-N-((5-cyclohexylpyridin-2-yl)methyl)-N-(2-(hydroxymethyl)-1-oxo-1,2-dihydroisoquinolin-6-yl)azetidine-2-carboxamide (75.6 mg, 0.116 mmol) in dichloromethane (1.8 mL) was added at 0 degrees C. isopropylamine (0.019 mL, 0.22 mmol) under argon. The mixture was stirred at 0 degrees C. for 8 h. A solution of 10% HOAc/NaOAc in water (1.7 mL) was added at 0 degrees C., and the mixture was extracted with dichloromethane (2×). The extract was washed with water, dried (Na2SO4) and concentrated. Purification by preparative thin layer chromatography (3:7 hexane/ethyl acetate with 3% methanol) gave the product of Example 2: (R)-1-((3-cyano-4,5-difluorophenyl)sulfonyl)-N-((5-cyclohexylpyridin-2-yl)methyl)-N-(1-oxo-1,2-dihydroisoquinolin-6-yl)azetidine-2-carboxamide (43 mg, 60% yield) 1H NMR (300 MHz, Chloroform-d) δ 11.22 (bs, 1H), 8.50-8.32 (m, 2H), 8.23-8.03 (m, 2H), 7.70-7.49 (m, 3H), 7.49-7.37 (m, 1H), 7.35-7.25 (m, 1H), 5.25-4.88 (m, 3H), 4.16-3.93 (m, 1H), 3.80-3.56 (m, 1H), 2.71-2.25 (m, 2H), 2.11-1.56 (m, 6H), 1.55-1.14 (m, 5H); and the product of Example 3: (R)-1-((3-cyano-5-fluoro-4-(isopropylamino)phenyl)sulfonyl)-N-((5-cyclohexylpyridin-2-yl)methyl)-N-(1-oxo-1,2-dihydroisoquinolin-6-yl)azetidine-2-carboxamide (22 mg, 28% yield) 1H NMR (300 MHz, Chloroform-d) δ11.19 (bs, 1H), 8.47-8.32 (m, 2H), 8.22-8.01 (m, 2H), 7.80-7.32 (m, 5H), 5.25-4.97 (m, 2H), 4.81 (bs, 1H), 4.70 (dd, J=8.7, 4.5 Hz, 1H), 4.54-4.33 (m, 1H), 3.94-3.75 (m, 1H), 3.73-3.54 (m, 1H), 2.67-2.27 (m, 2H), 2.01-1.59 (m, 6H), 1.55-1.17 (m, 5H), 1.33 (d, J=6.1 Hz, 6H).

### Example 4

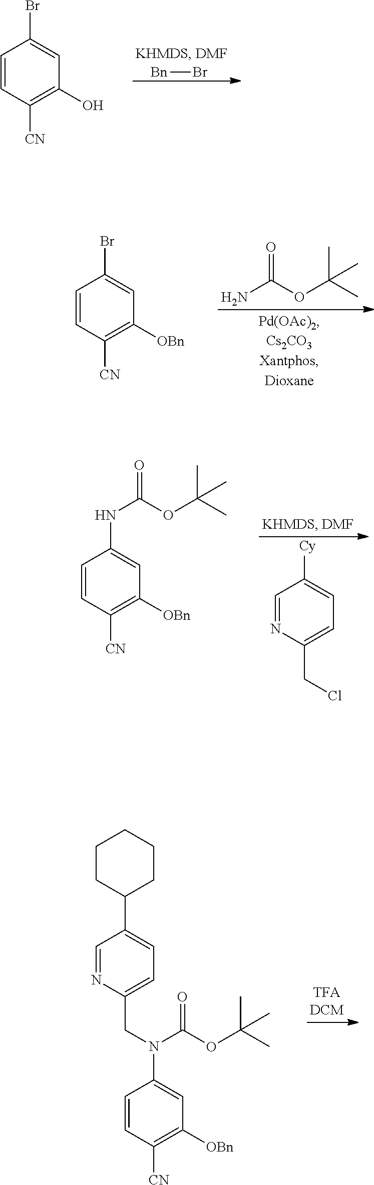

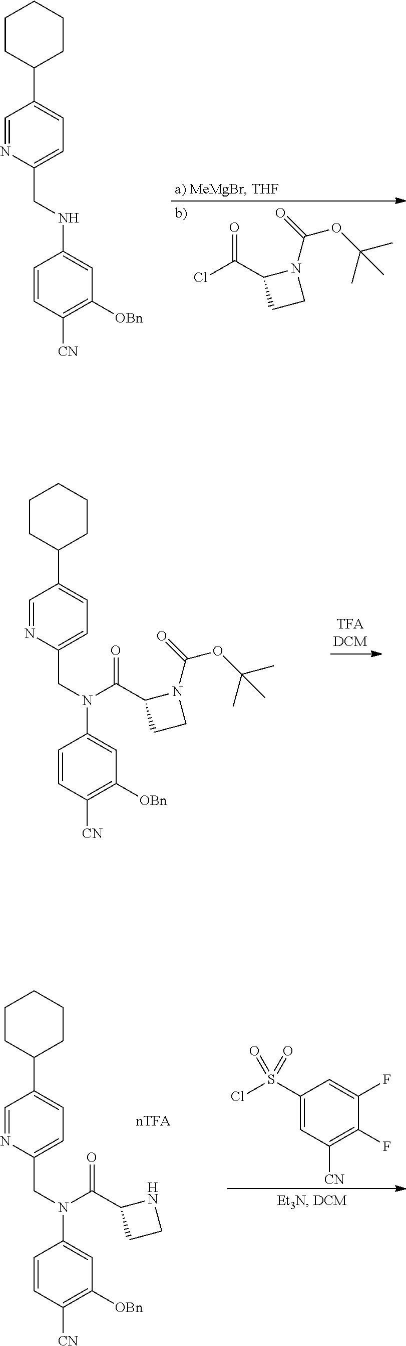

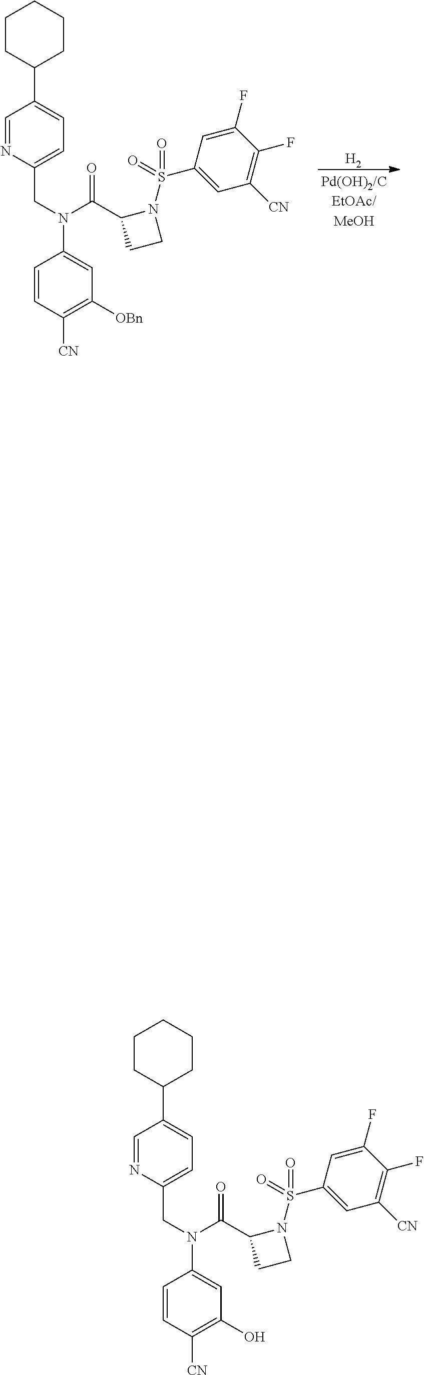 [SMILES: N#Cc1ccc(N(Cc2ccc(C3CCCCC3)cn2)C(=O)[C@H]2CCN2S(=O)(=O)c2cc(F)c(F)c(C#N)c2)cc1OCc1ccccc1 ; "H2" ; N#Cc1ccc(N(Cc2ccc(C3CCCCC3)cn2)C(=O)[C@H]2CCN2S(=O)(=O)c2cc(F)c(F)c(C#N)c2)cc1O]

### (R)—N-(4-Cyano-3-hydroxyphenyl)-1-((3-cyano-4,5-difluorophenyl)sulfonyl)-N-((5-cyclohexylpyridin-2-yl)methyl)azetidine-2-carboxamide

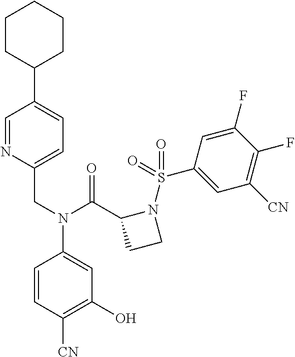 [SMILES: N#Cc1ccc(N(Cc2ccc(C3CCCCC3)cn2)C(=O)[C@H]2CCN2S(=O)(=O)c2cc(F)c(F)c(C#N)c2)cc1O]

Step 1. To a solution of 4-bromo-2-hydroxybenzonitrile (507 mg, 2.56 mmol) in DMF (5 mL) was added KHMDS (1M in THF, 3.07 mL, 3.07 mmol) at 0 degrees C. under argon. After 10 minutes, benzyl bromide (0.32 mL) was added dropwise at 0 degrees C. The mixture was allowed to reach room temperature and stirred for 5 h. The mixture was poured onto cold aqueous saturated ammonium chloride and extracted with ethyl acetate (3×). The extract was washed with water, brine, dried (Na2SO4) and concentrated. The crude solid was triturated with a mixture of dichloromethane (ca. 3 mL) and hexane (ca. 12 mL). The solid residue was mainly recovered starting material (ca. 128 mg). The filtrate was concentrated to dryness to obtain 2-(benzyloxy)-4-bromobenzonitrile as an off-white solid (493 mg, 67% yield). 1H NMR (300 MHz, Chloroform-d) δ 7.50-7.36 (m, 5H), 7.30-7.25 (m, 1H), 7.22-7.17 (m, 2H), 5.22 (s, 2H).

Step 2. A mixture of 2-(benzyloxy)-4-bromobenzonitrile (489 mg, 1.70 mmol), Xantphos (49.2 mg, 0.085 mmol), tert-buty carbamate (298 mg, 2.55 mmol), palladium acetate (19.1 mg, 0.085 mmol) and cesium carbonate (1.11 g, 3.39 mmol) was thoroughly flushed with argon. Dioxane (20 mL) was added through a septum, and the mixture was heated at 100 degrees C. (oil bath temperature) for 18 h. After cooling, aqueous saturated ammonium chloride was added, and the mixture was extracted with ethyl acetate (2×). The extract was washed with brine, dried (Na2SO4) and concentrated. Purification by flash column chromatography (3:7 hexane/ethyl acetate) gave tert-butyl (3-(benzyloxy)-4-cyanophenyl)carbamate (542 mg, 98% yield) as a pale yellow solid. 1H NMR (300 MHz, Chloroform-d) δ 7.59-7.30 (m, 6H), 6.84-6.69 (m, 2H), 5.21 (s, 2H), 1.54 (s, 9H).

Step 3. Preparation by a similar procedure to Example 2, step 3, starting from tert-butyl (3-(benzyloxy)-4-cyanophenyl)carbamate (535 mg, 1.65 mmol) to obtain tert-butyl (3-(benzyloxy)-4-cyanophenyl)((5-cyclohexylpyridin-2-yl)methyl)carbamate (775 mg, 94% yield) as a pale yellow oil. 1H NMR (300 MHz, Chloroform-d) δ 8.43 (d, J=2.1 Hz, 1H), 7.56-7.44 (m, 2H), 7.43-7.24 (m, 5H), 7.23-7.11 (m, 2H), 7.01 (dd, J=8.5, 1.9 Hz, 1H), 5.15 (s, 2H), 4.90 (s, 2H), 2.62-2.49 (s, 1H), 1.95-1.73 (m, 5H), 1.58-1.22 (m, 5H), 1.42 (s, 9H).

Step 4. To a solution of tert-butyl (3-(benzyloxy)-4-cyanophenyl)((5-cyclohexylpyridin-2-yl)methyl)carbamate (768 mg, 1.54 mmol) in dichloromethane (24 mL) was added at 0 degrees C. trifluoroacetic acid (8 mL) under argon. The mixture was stirred at 0 degrees C. for 6 h. Aqueous saturated sodium bicarbonate was added to pH 7-8. The mixture was extracted with dichloromethane (2×). The extract was dried (Na2SO4) and concentrated. Purification by flash column chromatography (75:25 to 6:4 hexane/ethyl acetate) gave 2-(benzyloxy)-4-(((5-cyclohexylpyridin-2-yl)methyl)amino)benzonitrile (495 mg, 81% yield) as colorless oil. 1H NMR (300 MHz, Chloroform-d) δ 8.44 (d, J=2.3 Hz, 1H), 7.57-7.24 (m, 7H), 7.19 (d, J=7.8 Hz, 1H), 6.31-6.17 (m, 2H), 5.51 (bs, 1H), 5.16 (s, 2H), 4.46-4.37 (m, 2H), 2.63-2.49 (m, 1H), 1.97-1.73 (m, 5H), 1.54-1.22 (m, 5H).

Step 5. Preparation by a similar procedure to Example 2, step 5, starting from 2-(benzyloxy)-4-(((5-cyclohexylpyridin-2-yl)methyl)amino)benzonitrile (479 mg, 1.21 mmol) to obtain tert-butyl (R)-2-((3-(benzyloxy)-4-cyanophenyl)((5-cyclohexylpyridin-2-yl)methyl)carbamoyl)azetidine-1-carboxylate (358 mg, 51% yield) as a light yellow foam. 1H NMR (300 MHz, Chloroform-d) δ 8.34 (s, 1H), 7.67-7.21 (m, 9H), 7.05-6.85 (m, 1H), 5.29-5.08 (m, 2H), 5.06-4.83 (m, 2H), 4.73-4.46 (m, 1H), 4.13-4.00 (m, 1H), 3.83-3.69 (m, 1H), 2.61-2.44 (m, 1H), 2.19-1.73 (m, 7H), 1.62-1.11 (m, 14H).

Step 6. To a solution of 2-(benzyloxy)-4-(((5-cyclohexylpyridin-2-yl)methyl)amino)benzonitrile (170 mg, 0.29 mmol) in dichloromethane (2 mL) was added trifluoroacetic acid (1 mL) under argon. The mixture was stirred for 2 h, and concentrated. Dichloroethane (2×ca. 3 mL) was added, and the mixture was concentrated to dryness to obtain crude (R)—N-(3-(benzyloxy)-4-cyanophenyl)-N-((5-cyclohexylpyridin-2-yl)methyl)azetidine-2-carb oxamide as TFA salt (274 mg), used as such for next reaction.

Step 7. To a solution of (R)—N-(3-(benzyloxy)-4-cyanophenyl)-N-((5-cyclohexylpyridin-2-yl)methyl)azetidine-2-carboxamide TFA salt (274 mg) in dichloromethane (6 mL) was added triethylamine (0.162 mL, 1.17 mmol) and stirred for 10 minutes. Powder 3-cyano-4,5-difluorobenzenesulfonyl chloride (98.7 mg, 0.31 mmol) was then added under argon. The mixture was stirred at room temperature for 2.5 h, then washed with water, dried (Na2SO4) and concentrated. Purification by flash column chromatography (6:4 hexane/ethyl acetate) gave (R)—N-(3-(benzyloxy)-4-cyanophenyl)-1-((3-cyano-4,5-difluorophenyl)sulfonyl)-N-((5-cyclohexylpyridin-2-yl)methyl)azetidine-2-carboxamide (170 mg, 85% for two steps) as pale yellow foam. 1H NMR (300 MHz, Chloroform-d) δ 8.39 (d, J=2.2 Hz, 1H), 8.21-8.03 (m, 2H), 7.66-7.48 (m, 2H), 7.45-7.32 (m, 5H), 7.26-7.15 (m, 1H), 7.03 (d, J=1.8 Hz, 1H), 6.91 (dd, J=8.2, 1.8 Hz, 1H), 5.25 (d, J=12.6 Hz, 1H), 5.14 (d, J=12.6 Hz, 1H), 5.05-4.84 (m, 1H), 4.98 (d, J=15.2 Hz, 1H), 4.84 (d, J=15.2 Hz, 1H), 4.02-3.90 (m, 1H), 3.64-3.50 (m, 1H), 2.62-2.46 (m, 1H), 2.20-1.99 (m, 2H), 1.95-1.73 (m, 5H), 1.52-1.17 (m, 5H).

Step 8. To a solution of (R)—N-(3-(benzyloxy)-4-cyanophenyl)-1-((3-cyano-4,5-difluorophenyl)sulfonyl)-N-((5-cyclohexylpyridin-2-yl)methyl)azetidine-2-carboxamide (165 mg, 0.24 mmol) in ethyl acetate (2.7 mL) and methanol (2.7 mL) was added 20% Pd(OH)₂ on carbon (50% water, 21.5 mg). A hydrogen atmosphere was set, and the mixture was stirred for 24 h. The mixture was filtered, and filtrate was concentrated. Purification by flash column chromatography (4:6 hexane ethyl acetate to 3:7 hexane/ethylacetate with 4% methanol) gave (R)—N-(4-cyano-3-hydroxyphenyl)-1-((3-cyano-4,5-difluorophenyl)sulfonyl)-N-((5-cyclohex ylpyridin-2-yl)methyl)azetidine-2-carboxamide (69 mg, 50% yield). 1H NMR (300 MHz, Chloroform-d) δ 8.30 (s, 1H), 8.18-7.99 (m, 2H), 7.76-7.69 (m, 2H), 7.56 (d, J=8.2 Hz, 1H), 6.64 (dd, J=8.2, 1.9 Hz, 1H), 6.55-6.47 (m, 1H), 5.23 (d, J=14.3 Hz, 1H), 4.95 (t, J=8.2 Hz, 1H), 4.79 (d, J=14.3 Hz, 1H), 4.00 (q, J=7.9 Hz, 1H), 3.75-3.62 (m, 1H), 2.61-2.42 (m, 1H), 2.41-2.20 (m, 1H), 2.03-1.55 (m, 6H), 1.53-1.02 (m, 5H).

### Example 5

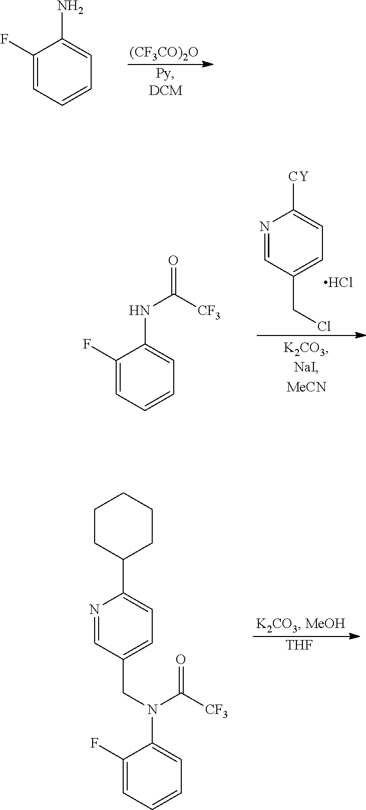

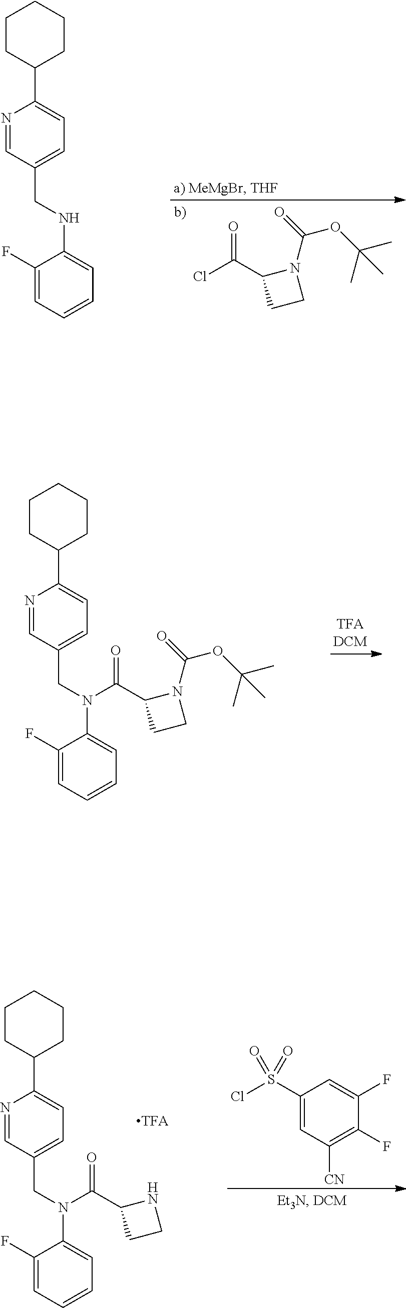

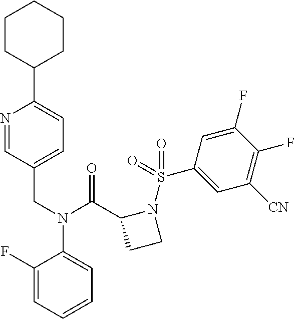 [SMILES: N#Cc1cc(S(=O)(=O)N2CC[C@@H]2C(=O)N(Cc2ccc(C3CCCCC3)nc2)c2ccccc2F)cc(F)c1F]

### (R)-1-((3-Cyano-4,5-difluorophenyl)sulfonyl)-N-((6-cyclohexylpyridin-3-yl)methyl)-N-(2-fluorophenyl)azetidine-2-carboxamide

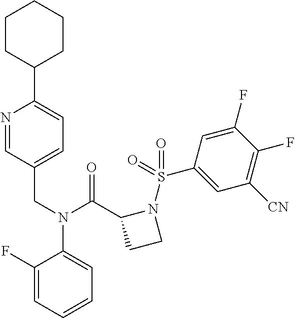 [SMILES: N#Cc1cc(S(=O)(=O)N2CC[C@@H]2C(=O)N(Cc2ccc(C3CCCCC3)nc2)c2ccccc2F)cc(F)c1F]

Step 1. To a solution of 2-fluoroaniline (1.01 g, 9.1 mmol) in dichloromethane (15 mL) was added at 0 degrees C. pyridine (1.61 mL) followed by trifluoroacetic anhydride (1.42 mL, 10.1 mmol) under argon. The mixture was allowed to reach room temperature and stirred for 1.5 h. The mixture was diluted with dichloromethane and washed with aqueous KHSO4/Na2SO4, saturated aqueous sodium bicarbonate, dried (Na2SO4) and concentrated. Purification by flash column chromatography (85:15 hexane/ethyl acetate) gave 2,2,2-trifluoro-N-(2-fluorophenyl)acetamide (1.49 g, 79% yield). 1H NMR (300 MHz, Chloroform-d) δ 8.35-8.23 (m, 1H), 8.19-8.02 (broad, 1H), 7.27-7.13 (m, 3H), 1.59 (s, 3H).

Step 2. To 2,2,2-trifluoro-N-(2-fluorophenyl)acetamide (374 mg, 1.81 mmol), 5-(chloromethyl)-2-cyclohexylpyridine hydrochloride (561 mg, 2.28 mmol) and sodium iodide (16 mg, 0.11 mmol) was added acetonitrile (23 mL) under argon. Potassium carbonate (749 mg, 5.42 mmol) was added, and the mixture was heated at 65 degrees C. (oil bath temperature) overnight. After cooling, saturated aqueous ammonium chloride was added, and the mixture was extracted with ethyl acetate (2×). The extract was washed with brine, dried (Na2SO4) and concentrated. Purification by flash column chromatography (9:1 to 7:3 hexane/ethyl acetate) gave N-((6-cyclohexylpyridin-3-yl)methyl)-2,2,2-trifluoro-N-(2-fluorophenyl)acetamide (353 mg, 51% yield) as a yellow oil. 1H NMR (300 MHz, Chloroform-d) δ 8.22 (d, J=2.0 Hz, 1H), 7.59 (dd, J=8.1, 2.4 Hz, 1H), 7.41 (dddd, J=8.4, 7.5, 5.0, 1.8 Hz, 1H), 7.23-7.05 (m, 3H), 7.01-6.88 (m, 1H), 5.25 (d, J=14.3 Hz, 1H), 4.48 (d, J=14.3 Hz, 1H), 2.69 (tt, J=11.6, 3.3 Hz, 1H), 2.05-1.69 (m, 5H), 1.59-1.32 (m, 5H).

Step 3. To a solution of N-((6-cyclohexylpyridin-3-yl)methyl)-2,2,2-trifluoro-N-(2-fluorophenyl)acetamide (450 mg, 1.18 mmol) in THF (6.1 mL) and methanol (7.4 mL) was added potassium carbonate (327 mg, 2.36 mmol) under argon. The mixture was stirred at room temperature for 2 h, then poured onto saturated aqueous ammonium chloride. The mixture was extracted with ethyl acetate (2×). The extract was washed with brine, dried (Na2SO4) and concentrated. Purification by flash column chromatography (8:2 hexane/ethyl acetate) gave N-((6-cyclohexylpyridin-3-yl)methyl)-2-fluoroaniline (243 mg, 72% yield). 1H NMR (300 MHz, Chloroform-d) δ 8.55 (d, J=2.0 Hz, 1H), 7.64 (dd, J=8.0, 2.4 Hz, 1H), 7.16 (d, J=8.0 Hz, 1H), 7.07-6.93 (m, 2H), 6.76-6.60 (m, 2H), 4.36 (s, 2H), 4.29 (bs, 1H), 2.72 (tt, J=11.6, 3.4 Hz, 1H), 2.02-1.71 (m, 5H), 1.59-1.31 (m, 5H).

Step 4. Preparation by a similar procedure to Example 2, step 5, starting from N-((6-cyclohexylpyridin-3-yl)methyl)-2-fluoroaniline (243 mg, 0.85 mmol) to obtain tert-butyl (R)-2-(((6-cyclohexylpyridin-3-yl)methyl)(2-fluorophenyl)carbamoyl)azetidine-1-carboxylat e (292 mg, 73% yield). 1H NMR (300 MHz, Chloroform-d) δ 8.29-8.17 (m, 1H), 7.76-7.59 (m, 1H), 7.40-7.30 (m, 1H), 7.23-6.74 (m, 4H), 5.31-5.06 (m, 1H), 4.69-4.39 (m, 2H), 4.25-3.84 (m, 1H), 3.83-3.63 (m, 1H), 2.78-2.58 (m, 1H), 2.56-2.00 (m, 2H), 1.97-1.67 (m, 5H), 1.61-1.18 (m, 5H), 1.43 (s, 9H).

Step 5. Preparation by a similar procedure to Example 4, step 6, starting from tert-butyl (R)-2-(((6-cyclohexylpyridin-3-yl)methyl)(2-fluorophenyl)carbamoyl)azetidine-1-carboxylat e (292 mg, 0.624 mmol) to obtain (R)—N-((6-cyclohexylpyridin-3-yl)methyl)-N-(2-fluorophenyl)azetidine-2-carboxamide (273 mg, 91%). 1H NMR (300 MHz, Chloroform-d) δ 8.31-8.23 (m, 1H), 7.61-7.53 (m, 1H), 7.52-7.39 (m, 1H), 7.26-7.11 (m, 3H), 7.05-6.87 (m, 1H), 5.26 (d, J=14.4 Hz, 1H), 5.12-4.92 (m, 1H), 4.50 and 5.06 (two doublets because of rotamers, J=14.4 Hz, 1H), 4.99 and 4.85 (two triplets because of rotamers, J=8.9 Hz, 1H), 4.71 and 4.50 (two doublets because of rotamers, J=14.4 Hz, 1H), 4.23-4.10 (m, 1H), 3.99-3.84 (m, 1H), 2.77-2.63 (m, 1H), 2.59-2.43 (m, 1H), 2.42-2.27 (m, 1H), 2.03-1.69 (m, 5H), 1.59-1.21 (m, 5H).

Step 6. Preparation by a similar procedure to Example 4, step 7, starting from (R)—N-((6-cyclohexylpyridin-3-yl)methyl)-N-(2-fluorophenyl)azetidine-2-carboxamide (273 mg, 0.566 mmol) to obtain (R)-1-((3-cyano-4,5-difluorophenyl)sulfonyl)-N-((6-cyclohexylpyridin-3-yl)methyl)-N-(2-fluorophenyl)azetidine-2-carboxamide (235 mg, 73% yield). 1H NMR (300 MHz, Chloroform-d) δ 8.21 (s, 1H), 8.26-7.99 (m, 2H), 7.64-7.51 (m, 1H), 7.49-7.36 (m, 1H), 7.32-6.82 (m, 4H), 5.23-5.02 (m, 1H), 4.92-4.76 (m, 1H), 4.60-4.47 (m, 1H), 4.10-3.90 (m, 1H), 3.74-3.61 (m, 1H), 2.76-2.70 (m, 1H), 2.46-2.13 (m, 2H), 2.01-1.69 (m, 5H), 1.58-1.27 (m, 5H).

### Example 6

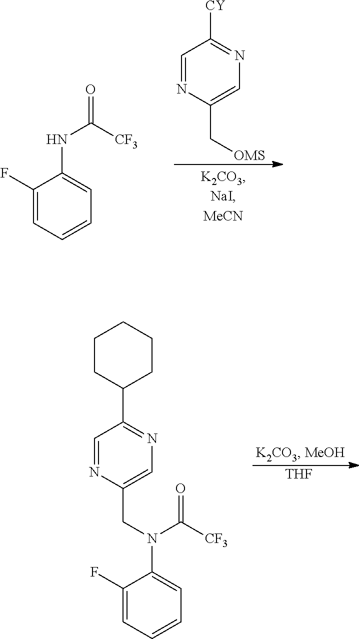

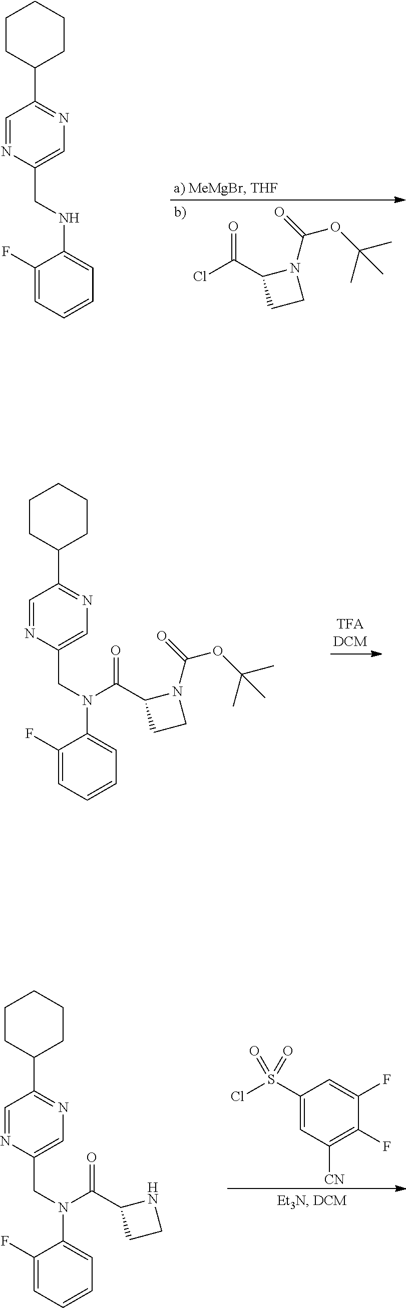

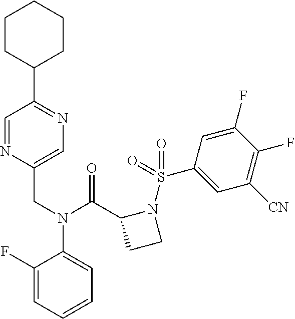 [SMILES: N#Cc1cc(S(=O)(=O)N2CC[C@@H]2C(=O)N(Cc2cnc(C3CCCCC3)cn2)c2ccccc2F)cc(F)c1F]

### (R)-1-((3-Cyano-4,5-difluorophenyl)sulfonyl)-N-((5-cyclohexylpyrazin-2-yl)methyl)-N-(2-fluorophenyl)azetidine-2-carboxamide

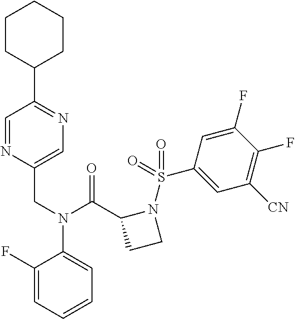 [SMILES: N#Cc1cc(S(=O)(=O)N2CC[C@@H]2C(=O)N(Cc2cnc(C3CCCCC3)cn2)c2ccccc2F)cc(F)c1F]

Step 1. To 2,2,2-trifluoro-N-(2-fluorophenyl)acetamide (36 mg, 0.174 mmol), (5-cyclohexylpyrazin-2-yl)methyl methanesulfonate (61 mg, 0.226 mmol) and sodium iodide (ca. 5.2 mg, 0.035 mmol) was added under argon acetonitrile (3 mL). Potassium carbonate (96.2 mg, 0.696 mmol) was added and the mixture was heated at 65 degrees C. for 22 h. After cooling, aqueous saturated ammonium chloride was added, and the mixture was extracted with ethyl acetate (2×). The extract was washed with brine, dried (Na2SO4), and concentrated. Purification by flash column chromatography (85:15 hexane/ethyl acetate) gave N-((5-cyclohexylpyrazin-2-yl)methyl)-2,2,2-trifluoro-N-(2-fluorophenyl)acetamide (67.5 mg, 99% yield) as a white solid. 1H NMR (300 MHz, Chloroform-d) δ 8.53 (d, J=1.5 Hz, 1H), 8.39 (d, J=1.5 Hz, 1H), 7.48-7.34 (m, 1H), 7.33-7.20 (m, 1H), 7.25-7.08 (m, 2H), 5.39 (d, J=14.9 Hz, 1H), 4.64 (d, J=14.9 Hz, 1H), 2.82-2.67 (m, 1H), 2.02-2.72 (m, 5H), 1.66-1.20 (m, 5H).

Step 2. To a solution of N-((5-cyclohexylpyrazin-2-yl)methyl)-2,2,2-trifluoro-N-(2-fluorophenyl)acetamide (63.5 mg, 0.166 mmol) in THF (0.9 mL) and methanol (1.0 mL) was added potassium carbonate (46 mg, 0.333 mL) under argon. The mixture was stirred at room temperature for 2 h. Aqueous saturated ammonium chloride was added, and the mixture was extracted with ethyl acetate (2×). The extract was washed with brine, dried (Na2SO4) and concentrated. Purification by flash column chromatography (85:15 hexane/ethyl acetate) gave N-((5-cyclohexylpyrazin-2-yl)methyl)-2-fluoroaniline (44.8 mg, 95% yield) as a light yellow solid. 1H NMR (300 MHz, Chloroform-d) δ 8.56 (d, J=1.5 Hz, 1H), 8.45 (d, J=1.5 Hz, 1H), 7.09-6.94 (m, 2H), 6.79-6.61 (m, 2H), 4.88 (bs, 1H), 4.52 (d, J=5.5 Hz, 2H), 2.85-2.68 (m, 1H), 2.02-1.71 (m, 5H), 1.68-1.21 (m, 5H).

Step 3. To a solution of N-((5-cyclohexylpyrazin-2-yl)methyl)-2-fluoroaniline (44.8 mg, 0.157 mmol) in THF (1.2 mL) was added at 0 degrees C. under argon methylmagnesium bromide (1.4 M in THF, 0.28 mL, 0.40 mmol). The mixture was stirred at 0 degrees C. for 15 minutes. A solution of tert-butyl (R)-2-(chlorocarbonyl)azetidine-1-carboxylate (freshly prepared from (R)-1-(tert-butoxycarbonyl)azetidine-2-carboxylic acid (65.8 mg, 0.327 mmol), according to Example 2, step 5a) in THF (1.2 mL) at 0 degrees C. The mixture was allowed to reach room temperature and stirred for 1 h. Cold aqueous saturated ammonium chloride was added and the mixture was extracted with ethyl acetate (2×). The extract was washed with brine, dried (Na2SO4) and concentrated. Purification by flash column chromatography (7:3 to 1:1 hexane/ethyl acetate) gave tert-butyl (R)-2-(((5-cyclohexylpyrazin-2-yl)methyl)(2-fluorophenyl)carbamoyl)azetidine-1-carboxylat e (58 mg, 79% yield) as a light yellow oil. 1H NMR (300 MHz, Chloroform-d) δ 8.73-8.57 (m, 1H), 8.36-8.28 (m, 1H), 7.44-7.30 (m, 1H), 7.25-7.03 (m, 3H), 4.61-4.40 (m, 1H), 4.21-3.97 (m, 1H), 3.76 (q, J=7.7 Hz, 1H), 2.80-2.64 (m, 1H), 2.27-2.07 (m, 2H), 1.96-1.22 (m, 10H), 1.43 (s, 9H).

Step 4. To a solution of tert-butyl (R)-2-(((5-cyclohexylpyrazin-2-yl)methyl)(2-fluorophenyl)carbamoyl)azetidine-1-carboxylate (55 mg, 0.117 mmol) in dichloromethane (1 mL) was added trifluoroacetic acid (0.5 mL) under argon. The mixture was stirred for 2 h, then concentrated to dryness. Dichloroethane (2×) was added and the mixture was concentrated back. Purification by flash column chromatography (20/15/0.1 DCM/MeOH/28% aq NH4OH) gave (R)—N-((5-cyclohexylpyrazin-2-yl)methyl)-N-(2-fluorophenyl)azetidine-2-carboxamide (41.5 mg, 96% yield). 1H NMR (300 MHz, Chloroform-d) δ 8.55 (s, 1H), 8.36 (s, 1H), 7.42-7.31 (m, 1H), 7.30-7.05 (m, 3H), 5.48-5.25 (m, 1H), 4.76-4.57 (m, 1H), 4.48-4.17 (m, 1H), 3.65 (q, J=8.3 Hz, 1H), 3.57-3.39 (m, 1H), 2.80-2.65 (m, 1H), 2.46-2.16 (m, 2H), 1.99-1.69 (m, 5H), 1.65-1.24 (m, 5H).

Step 5. To a solution of (R)—N-((5-cyclohexylpyrazin-2-yl)methyl)-N-(2-fluorophenyl)azetidine-2-carboxamide (41.5 mg, 0.113 mmol) in dichloromethane (1.5 mL) was added triethylamine (0.047 mL, 0.339 mmol) followed by powder 3-cyano-4,5-difluorobenzenesulfonyl chloride (38.1 mg, 0.119 mmol) under argon. The mixture was stirred at room temperature for 2.5 h, then washed with water, dried (Na2SO4) and concentrated. Purification by flash column chromatography (7:3 hexane/acetone) gave (R)-1-((3-cyano-4,5-difluorophenyl)sulfonyl)-N-((5-cyclohexylpyrazin-2-yl)methyl)-N-(2-fluorophenyl)azetidine-2-carboxamide (44 mg, 68% yield) as a white foam. 1H NMR (300 MHz, Chloroform-d) δ 8.56-8.46 (m, 1H), 8.40-8.32 (m, 1H), 8.26-8.12 (m, 1H), 8.11-7.99 (m, 1H), 7.49-7.11 (m, 4H), 5.38-5.23 (m, 1H), 5.01-4.82 (m, 1H), 4.78-4.52 (m, 1H), 4.09-3.92 (m, 1H), 3.75-3.59 (m, 1H), 2.81-2.67 (m, 1H), 2.48-2.23 (m, 1H), 2.0-1.73 (m, 6H), 1.68-1.22 (m, 5H).

### Example 7

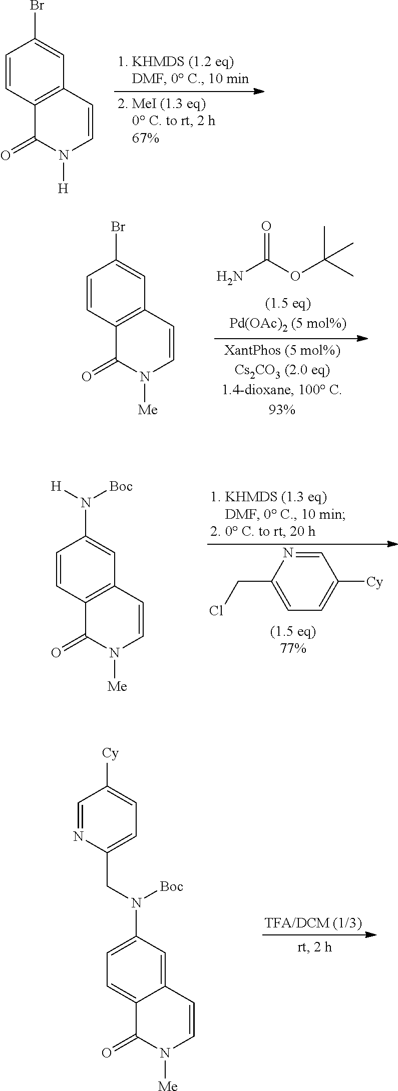

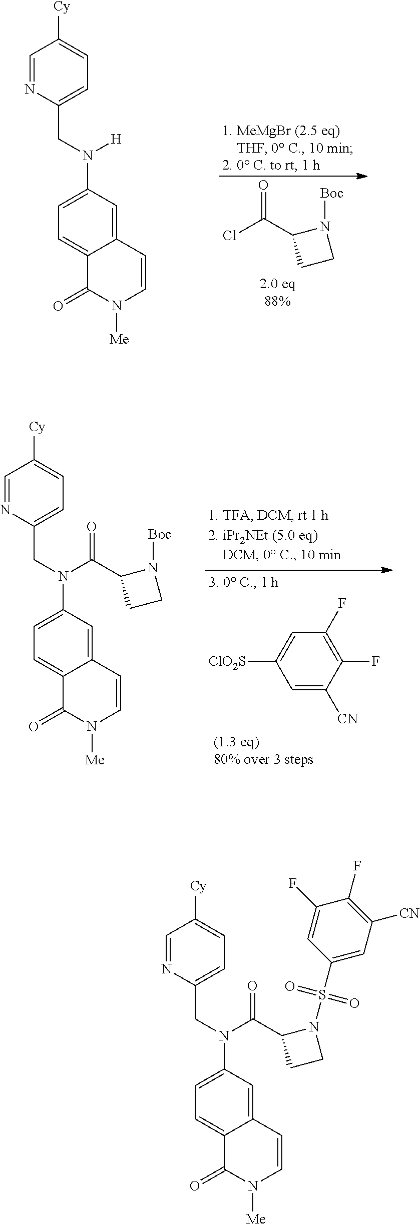

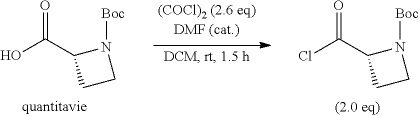 [SMILES: CC(C)(C)OC(=O)N1CC[C@@H]1C(=O)O ; CC(C)(C)OC(=O)N1CC[C@@H]1C(=O)Cl]

### (R)-1-((3-cyano-4,5-difluorophenyl)sulfonyl)-N-((5-cyclohexylpyridin-2-yl)methyl)-N-(2-methyl-1-oxo-1,2-dihydroisoquinolin-6-yl)azetidine-2-carboxamide

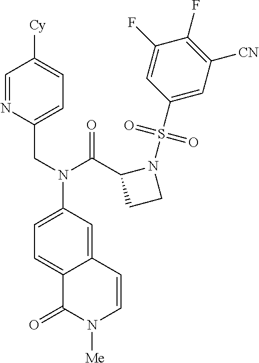

Step 1: To a solution of 6-bromoisoquinolin-1(2H)-one (1.00 g, 4.46 mmol, 1.0 equiv) in 20 mL DMF was added KHMDS (5.40 mL, 5.40 mmol, 1.2 equiv, 1.0 M in THF) at 0 degrees C. under Argon. After 10 min, MeI (0.36 mL, 5.80 mmol, 1.3 equiv) was added to the reaction mixture dropwise. The reaction was allowed to warm up to room temperature and stirred at room temperature for 2 h. Then the reaction was quenched with cold saturated NH4Cl aq. White precipitate was formed. The solid was filtered, washed with water and hexane, and dried under high vacuum to give 6-bromo-2-methylisoquinolin-1(2H)-one as white solid (705 mg, 67%). 1H NMR (300 MHz, CDCl3) δ 8.28 (d, J=8.6 Hz, 1H), 7.67 (d, J=1.9 Hz, 1H), 7.57 (dd, J=8.6, 1.9 Hz, 1H), 7.10 (d, J=7.3 Hz, 1H), 6.40 (d, J=7.3 Hz, 1H), 3.60 (s, 3H).

Step 2: 6-Bromo-2-methylisoquinolin-1(2H)-one (600 mg, 2.52 mmol, 1.0 equiv), tert-butyl carbamate (443 mg, 3.78 mmol, 1.5 equiv), and Cs2CO3 (1.64 g, 5.04 mmol, 2.0 equiv) were dissolved in 25 mL 1,4-dioxane. After 10 min, Pd(OAc)2 (28 mg, 0.13 mmol, 5 mol %) and XantPhos (73 mg, 0.13 mmol, 5 mol %) were added to the reaction under Argon. Then the reaction was heated at 100 degrees C. for 24 h. The reaction was quenched with water, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 2/1) to provide tert-butyl (2-methyl-1-oxo-1,2-dihydroisoquinolin-6-yl)carbamate as white solid (643 mg, 93%). 1H NMR (300 MHz, CDCl3) δ 8.36 (d, J=8.7 Hz, 1H), 7.84 (s, 1H), 7.24-7.16 (m, 1H), 7.04 (d, J=7.3 Hz, 1H), 6.79 (s, 1H), 6.45 (d, J=7.3 Hz, 1H), 3.59 (s, 3H), 1.54 (s, 9H).

Step 3: To a solution of tert-butyl (2-methyl-1-oxo-1,2-dihydroisoquinolin-6-yl)carbamate (686 mg, 2.50 mmol, 1.0 equiv) in 15 mL DMF was added KHMDS (3.5 mL, 3.50 mmol, 1.4 equiv, 1.0 M in THF) at 0 degrees C. dropwise under Argon. 10 min late, 2-(chloromethyl)-5-cyclohexylpyridine (8.0 mL, 4.00 mmol, 1.6 equiv, 0.5 M in toluene) was added to the reaction mixture. The reaction was allowed to warm up to room temperature and stirred at room temperature for 20 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 3/1) to provide tert-butyl ((5-cyclohexylpyridin-2-yl)methyl)(2-methyl-1-oxo-1,2-dihydroisoquinolin-6-yl)carbamate as light yellow oil (860 mg, 77%). 1H NMR (300 MHz, CDCl3) δ 8.40 (s, 1H), 8.32 (d, J=8.7 Hz, 1H), 7.67 (d, J=8.1 Hz, 1H), 7.47 (d, J=2.0 Hz, 1H), 7.43-7.35 (m, 2H), 7.04 (d, J=7.3 Hz, 1H), 6.42 (d, J=7.3 Hz, 1H), 5.17 (s, 2H), 3.57 (s, 3H), 2.56 (m, 1H), 1.82 (m, 6H), 1.46-1.34 (m, 13H).

Step 4: To a solution of tert-butyl ((5-cyclohexylpyridin-2-yl)methyl)(2-methyl-1-oxo-1,2-dihydroisoquinolin-6-yl)carbamate (750 mg, 1.67 mmol) in 20 mL DCM was added TFA (7.5 mL). the reaction was stirred at room temperature for 2 h. Then the reaction was concentrated under reduced pressure, then quenched with saturated NaHCO3 aq. to pH 8. The reaction was extracted with ethyl acetate (3×). The combined organic extracts were washed with saturated brine, dried over anhydrous sodium sulfate, and concentrated under reduced pressure. The resulting residue was dried under high vacuum to provide 6-(((5-cyclohexylpyridin-2-yl)methyl)amino)-2-methylisoquinolin-1(2H)-one as brown solid (580 mg, quantitative). 1H NMR (300 MHz, CDCl3) δ 8.45 (d, J=2.0 Hz, 1H), 8.20 (d, J=8.8 Hz, 1H), 7.67 (d, J=8.1 Hz, 1H), 7.40 (d, J=8.0 Hz, 1H), 6.95 (d, J=7.3 Hz, 1H), 6.84 (dd, J=8.8, 2.3 Hz, 1H), 6.53 (d, J=2.3 Hz, 1H), 6.28 (d, J=7.3 Hz, 1H), 5.61 (s, 1H), 4.62 (s, 2H), 3.53 (s, 3H), 2.57 (m, 1H), 2.01-1.73 (m, 6H), 1.50-1.35 (m, 4H).

Step 5a: To a solution of (R)-1-(tert-butoxycarbonyl)azetidine-2-carboxylic acid (173 mg, 0.86 mmol, 2.0 equiv) in 8 mL DCM was added DMF (1 drop, cat.) and oxalyl chloride (90 μL, 1.08 mmol, 2.5 equiv) dropwise under Argon. The reaction was stirred at room temperature for 1.5 h. Then the mixture was concentrated under reduced pressure, diluted with dry DCE and concentrated again. The resulting acid chloride was dried under high vacuum for 30 min and used directly for the next step.

Step 5b. To a solution of 6-(((5-cyclohexylpyridin-2-yl)methyl)amino)-2-methylisoquinolin-1(2H)-one (150 mg, 0.43 mmol, 1.0 equiv) in 4 mL THF was added MeMgBr (0.8 mL, 1.08 mmol, 2.5 equiv, 1.4 M in THF/toluene) at 0 degrees C. under Argon. 10 min later, a solution of the above acid chloride in 2 mL THF was added to the reaction. The reaction was allowed to warm up to room temperature and stirred for 1 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc/MeOH 1/2/5%) to provide tert-butyl (R)-2-(((5-cyclohexylpyridin-2-yl)methyl)(2-methyl-1-oxo-1,2-dihydroisoquinolin-6-yl)carbamoyl)azetidine-1-carboxylate as light yellow oil (197 mg, 86%). 1H NMR (300 MHz, CDCl3) δ 8.35 (d, J=8.4 Hz, 1H), 8.28 (s, 1H), 7.50-7.34 (m, 3H), 7.23 (s, 1H), 7.07 (d, J=7.3 Hz, 1H), 6.37 (d, J=7.4 Hz, 1H), 5.05 (s, 2H), 4.63-4.52 (m, 1H), 4.04-3.99 (m, 1H), 3.75-3.66 (m, 1H), 3.56 (s, 3H), 2.52-2.41 (m, 1H), 2.25-2.08 (m, 2H), 1.89-1.65 (m, 6H), 1.47-1.31 (m, 13H).

Step 6: To a solution of tert-butyl (R)-2-(((5-cyclohexylpyridin-2-yl)methyl)(2-methyl-1-oxo-1,2-dihydroisoquinolin-6-yl)carbamoyl)azetidine-1-carboxylate (190 mg, 0.36 mmol) in 6 mL DCM was added TFA (2 mL). the reaction was stirred at room temperature for 1 h. Then the reaction was concentrated under reduced pressure, diluted with dry DCE and concentrated again. The residue was dried under high vacuum for 30 min and used directly for the next step.

Step 7. To a solution of the above residue in 7 mL DCM was added DIPEA (0.3 mL, 1.79 mmol, 5.0 equiv) at 0 degrees C. under Argon. After 10 min, a solution of 3-cyano-4,5-difluorobenzenesulfonyl chloride (111 mg, 0.47 mmol, 1.3 equiv) in 2 mL DCM was added dropwise under Argon at 0 degrees C. The reaction was stirred at 0 degrees C. for 1 h. Then the reaction was quenched with saturated NH4Cl aq, extracted with DCM (3×). The combined organic extracts were washed with saturated brine, dried over anhydrous sodium sulfate, and concentrated under reduced pressure. The resulting residue was purified by flash chromatography (eluent: DCM/MeOH 80/1) to provide (R)-1-((3-cyano-4,5-difluorophenyl)sulfonyl)-N-((5-cyclohexylpyridin-2-yl)methyl)-N-(2-methyl-1-oxo-1,2-dihydroiso-quinolin-6-yl)azetidine-2-carboxamide as white solid (180 mg, 80%). 1H NMR (300 MHz, CDCl3) δ 8.43 (d, J=8.5 Hz, 1H), 8.37 (s, 1H), 8.23-8.12 (m, 1H), 8.06 (s, 1H), 7.52 (d, J=7.7 Hz, 1H), 7.41 (s, 1H), 7.29-7.19 (m, 2H), 7.15 (d, J=7.3 Hz, 1H), 6.45 (d, J=7.3 Hz, 1H), 5.03 (s, 2H), 5.03 (m, 1H), 4.03-3.91 (m, 1H), 3.62 (s, 3H), 3.62 (m, 1H), 3.53-3.44 (m, 1H), 2.60-2.46 (m, 1H), 2.45-2.29 (m, 1H), 1.92-1.77 (m, 6H), 1.50-1.35 (m, 4H). LRMS (ESI) m/z 632.3 [M+H]+; HRMS (ESI) m/z 632.2136 [M+H]+, 654.1952[M+Na]+; Purity 99%.

### Example 8

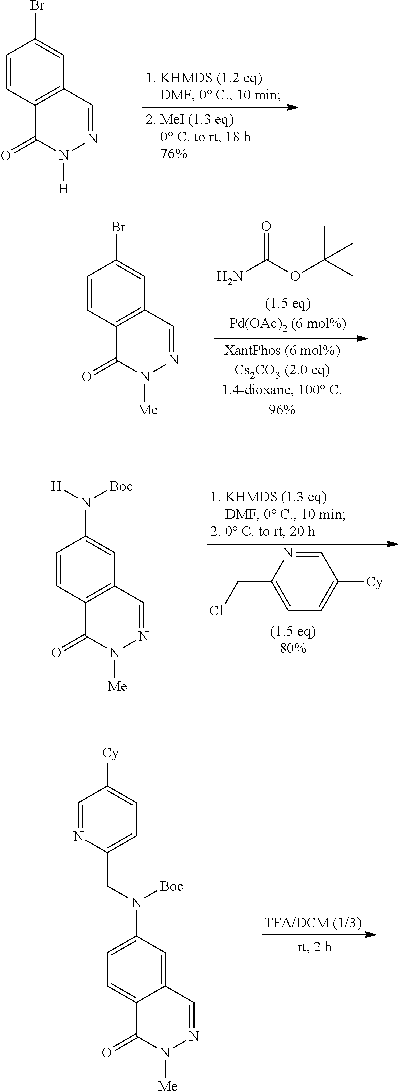

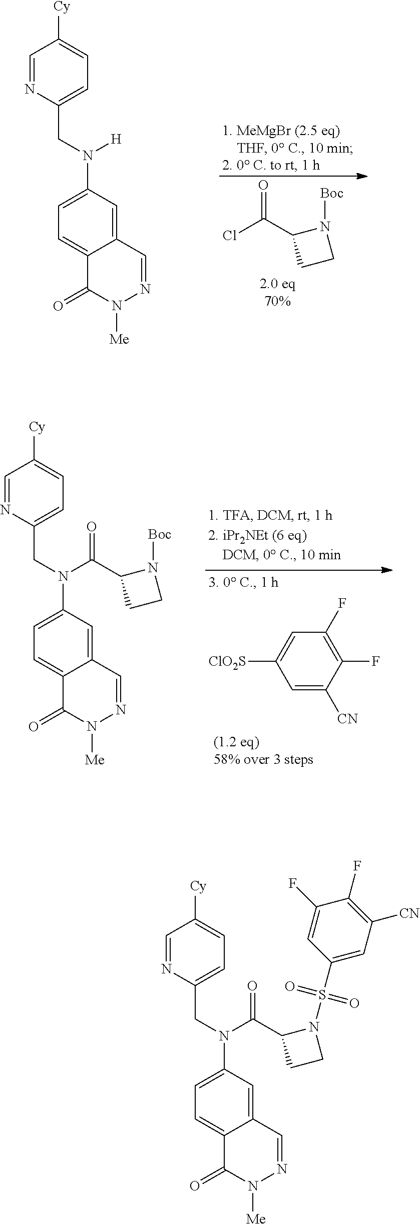

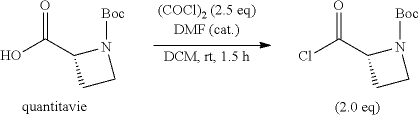 [SMILES: CC(C)(C)OC(=O)N1CC[C@@H]1C(=O)O ; CC(C)(C)OC(=O)N1CC[C@@H]1C(=O)Cl]

### (R)-1-((3-cyano-4,5-difluorophenyl)sulfonyl)-N-((5-cyclohexylpyridin-2-yl)methyl)-N-(3-methyl-4-oxo-3,4-dihydroquinazolin-7-yl)azetidine-2-carboxamide

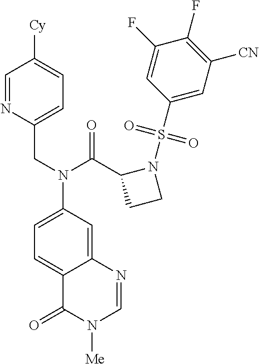

Step 1. Preparation by a similar procedure to Example 7, step 1, starting from 6-bromoisoquinolin-1(2H)-one to obtain 7-bromo-3-methylquinazolin-4(3H)-one (white solid, 76%). 1H NMR (300 MHz, CDCl3) δ 8.16 (d, J=8.5 Hz, 1H), 8.06 (s, 1H), 7.89 (d, J=1.8 Hz, 1H), 7.61 (dd, J=8.5, 1.9 Hz, 1H), 3.59 (s, 3H).

Step 2. Preparation by a similar procedure to Example 7, step 2, starting from 7-bromo-3-methylquinazolin-4(3H)-one to obtain tert-Butyl (3-methyl-4-oxo-3,4-dihydroquinazolin-7-yl)carbamate (white solid, 96%). 1H NMR (300 MHz, CDCl3) δ 8.54-8.44 (m, 1H), 8.27-8.18 (m, 1H), 7.78-7.67 (m, 2H), 7.01 (s, 1H), 3.64 (s, 3H), 1.54 (s, 9H).

Step 3. Preparation by a similar procedure to Example 7, step 3, starting from tert-Butyl (3-methyl-4-oxo-3,4-dihydroquinazolin-7-yl)carbamate to obtain tert-Butyl ((5-cyclohexylpyridin-2-yl)methyl)(3-methyl-4-oxo-3,4-dihydroquinazolin-7-yl)carbamate (yellow oil, 80%). 1H NMR (300 MHz, CDCl3) δ 8.44-8.40 (m, 1H), 8.26-8.20 (m, 1H), 8.03-7.99 (m, 1H), 7.63-7.53 (m, 3H), 7.32-7.28 (m, 1H), 5.10 (s, 2H), 3.57 (s, 3H), 2.61-2.49 (m, 1H), 1.95-1.71 (m, 6H), 1.47-1.35 (m, 13H).

Steps 4 and 5. Preparation by a similar procedure to Example 7, steps 4 and 5, starting from tert-Butyl ((5-cyclohexylpyridin-2-yl)methyl)(3-methyl-4-oxo-3,4-dihydroquinazolin-7-yl)carbamate to obtain tert-Butyl (R)-2-(((5-cyclohexylpyridin-2-yl)methyl)(3-methyl-4-oxo-3,4-dihydroquinazolin-7-yl)carbamoyl)azetidine-1-carboxylate (light yellow oil, 70%). 1H NMR (300 MHz, CDCl3) δ 8.35-8.24 (m, 2H), 8.04 (s, 1H), 7.60-7.33 (m, 4H), 5.08 (s, 2H), 4.69-4.55 (m, 1H), 4.09-4.03 (m, 1H), 3.79-3.70 (m, 1H), 3.59 (s, 3H), 2.54-2.44 (m, 1H), 2.27-2.12 (m, 2H), 1.91-1.70 (m, 6H), 1.49-1.33 (m, 13H).

Step 6. Preparation by a similar procedure to Example 7, step 6, starting from tert-Butyl (R)-2-(((5-cyclohexylpyridin-2-yl)methyl)(3-methyl-4-oxo-3,4-dihydroquinazolin-7-yl)carbamoyl)azetidine-1-carboxylate to obtain (R)-1-((3-cyano-4,5-difluorophenyl)sulfonyl)-N-((5-cyclohexylpyridin-2-yl)methyl)-N-(3-methyl-4-oxo-3,4-dihydroquinazolin-7-yl)azetidine-2-carboxamide (white solid, 58% over 3 steps). 1H NMR (300 MHz, CDCl3) δ 8.42-8.29 (m, 2H), 8.21-7.97 (m, 3H), 7.58 (s, 1H), 7.52 (d, J=7.7 Hz, 1H), 7.37 (d, J=8.3 Hz, 1H), 7.32-7.24 (m, 1H), 5.17-4.92 (m, 3H), 4.03-3.88 (m, 1H), 3.75-3.67 (m, 1H), 3.61 (s, 3H), 2.59-2.37 (m, 2H), 2.00-1.70 (m, 7H), 1.51-1.33 (m, 4H). LRMS (ESI) m/z 633.3 [M+H]+; HRMS (ESI) m/z 633.2093 [M+H]+, 655.1941 [M+Na]+; Purity 100%.

### Example 9

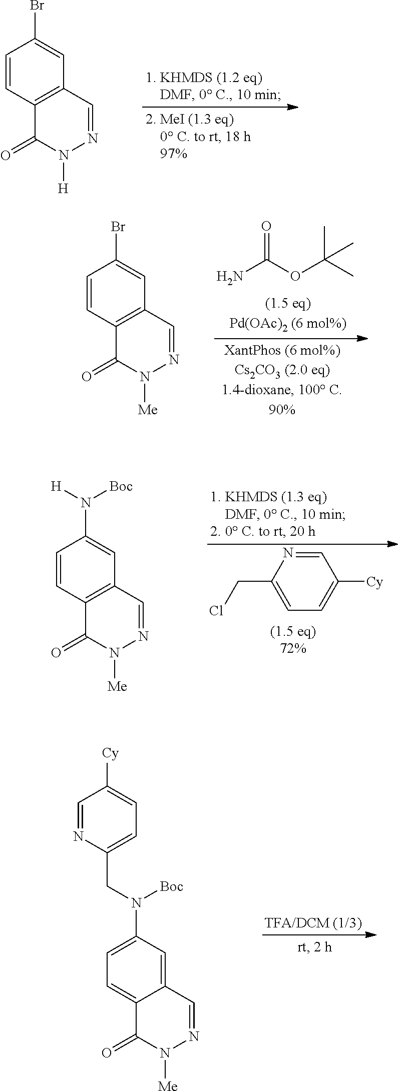

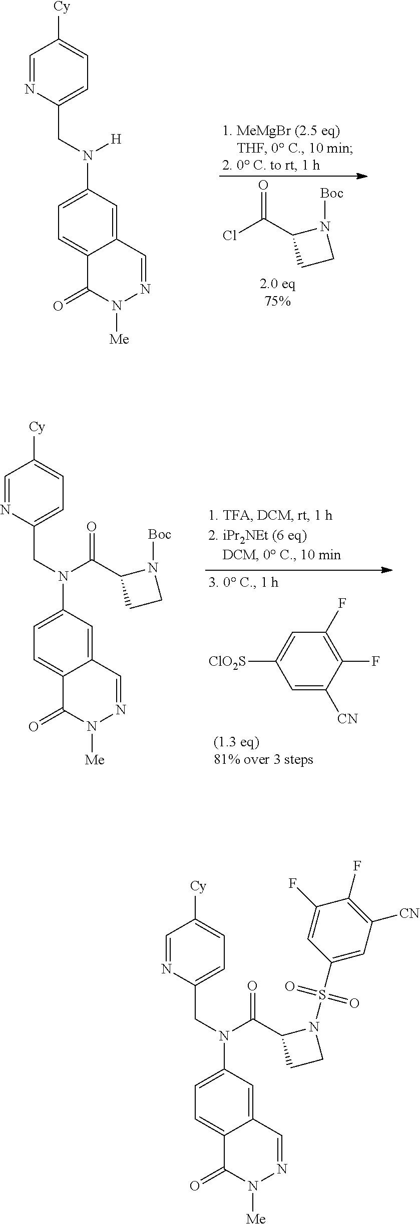

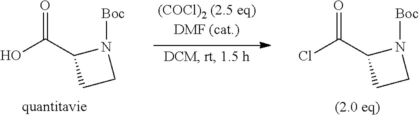 [SMILES: CC(C)(C)OC(=O)N1CC[C@@H]1C(=O)O ; CC(C)(C)OC(=O)N1CC[C@@H]1C(=O)Cl]

### (R)-1-((3-cyano-4,5-difluorophenyl)sulfonyl)-N-((5-cyclohexylpyridin-2-yl)methyl)-N-(2-methyl-1-oxo-1,2-dihydrophthalazin-6-yl)azetidine-2-carboxamide

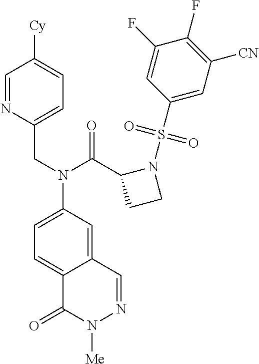

Step 1. Preparation by a similar procedure to Example 7, step 1, starting from 6-bromophthalazin-1(2H)-one to obtain 6-bromo-2-methylphthalazin-1(2H)-one (white solid, 97%). 1H NMR (300 MHz, CDCl3) δ 8.37-8.29 (m, 1H), 8.15-8.06 (m, 1H), 7.94-7.84 (m, 2H), 3.86 (s, 3H).

Step 2. Preparation by a similar procedure to Example 7, step 2, starting from 6-bromo-2-methylphthalazin-1(2H)-one to obtain tert-Butyl (2-methyl-1-oxo-1,2-dihydrophthalazin-6-yl)carbamate (white solid, 90%). 1H NMR (300 MHz, CDCl3) δ 8.32 (d, J=8.7 Hz, 1H), 8.22-8.02 (m, 2H), 7.44 (dd, J=8.7, 2.2 Hz, 1H), 7.05 (s, 1H), 3.83 (s, 3H), 1.53 (s, 9H).

Step 3. Preparation by a similar procedure to Example 7, step 3, starting from tert-Butyl (2-methyl-1-oxo-1,2-dihydrophthalazin-6-yl)carbamate to obtain tert-Butyl ((5-cyclohexylpyridin-2-yl)methyl)(2-methyl-1-oxo-1,2-dihydrophthalazin-6-yl)carbamate (yellow oil, 72%). 1H NMR (300 MHz, CDCl3) δ 8.42 (d, J=2.0 Hz, 1H), 8.32 (d, J=8.3 Hz, 1H), 8.06 (s, 1H), 7.74 (d, J=2.1 Hz, 1H), 7.71 (s, 1H), 7.60 (dd, J=8.0, 1.8 Hz, 1H), 7.30 (d, J=8.1 Hz, 1H), 5.11 (s, 2H), 3.82 (s, 3H), 2.61-2.51 (m, 1H), 1.99-1.71 (m, 6H), 1.52-1.35 (m, 13H).

Step 4. Preparation by a similar procedure to Example 7, step 4, starting from tert-Butyl ((5-cyclohexylpyridin-2-yl)methyl)(2-methyl-1-oxo-1,2-dihydrophthalazin-6-yl)carbamate to obtain 6-(((5-Cyclohexylpyridin-2-yl)methyl)amino)-2-methylphthalazin-1(2H)-one (light yellow solid, quantitative). 1H NMR (300 MHz, CDCl3) δ 8.47 (s, 1H), 8.25-8.16 (m, 1H), 8.00-7.94 (m, 1H), 7.76-7.67 (m, 1H), 7.42 (d, J=8.0 Hz, 1H), 7.14-7.08 (m, 1H), 6.68 (s, 1H), 5.99 (s, 1H), 4.64 (s, 2H), 3.80 (s, 3H), 2.67-2.54 (m, 1H), 1.94-1.74 (m, 6H), 1.47-1.38 (m, 4H).

Step 5. Preparation by a similar procedure to Example 7, step 5, starting from 6-(((5-Cyclohexylpyridin-2-yl)methyl)amino)-2-methylphthalazin-1(2H)-one to obtain tert-Butyl (R)-2-(((5-cyclohexylpyridin-2-yl)methyl)(2-methyl-1-oxo-1,2-dihydrophthalazin-6-yl)carbamoyl)azetidine-1-carboxylate (colorless oil, 75%). 1H NMR (300 MHz, CDCl3) δ 8.40 (d, J=8.3 Hz, 1H), 8.32 (s, 1H), 8.07 (s, 1H), 7.77-7.55 (m, 2H), 7.51 (dd, J=8.0, 2.1 Hz, 1H), 7.42 (d, J=8.2 Hz, 1H), 5.08 (s, 2H), 4.70-4.50 (m, 1H), 4.12-4.03 (m, 1H), 3.84 (s, 3H), 3.79-3.69 (m, 1H), 2.55-2.44 (m, 1H), 2.30-2.14 (m, 2H), 1.90-1.69 (m, 6H), 1.56-1.29 (m, 13H).

Step 6. Preparation by a similar procedure to Example 7, step 6 and 7, starting from tert-Butyl (R)-2-(((5-cyclohexylpyridin-2-yl)methyl)(2-methyl-1-oxo-1,2-dihydrophthalazin-6-yl)carbamoyl)azetidine-1-carboxylate to obtain (R)-1-((3-cyano-4,5-difluorophenyl)sulfonyl)-N-((5-cyclohexylpyridin-2-yl)methyl)-N-(2-methyl-1-oxo-1,2-dihydrophthalazin-6-yl)azetidine-2-carboxamide (white solid, 81%). 1H NMR (300 MHz, CDCl3) δ 8.45 (d, J=8.3 Hz, 1H), 8.38 (s, 1H), 8.22-8.01 (m, 3H), 7.66 (s, 1H), 7.57 (dd, J=22.0, 7.7 Hz, 2H), 7.28 (s, 1H), 5.16-4.90 (m, 3H), 4.07-3.95 (m, 1H), 3.87 (s, 3H), 3.71-3.59 (m, 1H), 2.62-2.46 (m, 1H), 2.45-2.30 (m, 1H), 2.00-1.71 (m, 7H), 1.49-1.34 (m, 4H). LRMS (ESI) m/z 633.3 [M+H]+; HRMS (ESI) m/z 633.2088 [M+H]+, 655.1908 [M+Na]+; Purity 100%.

### Example 10

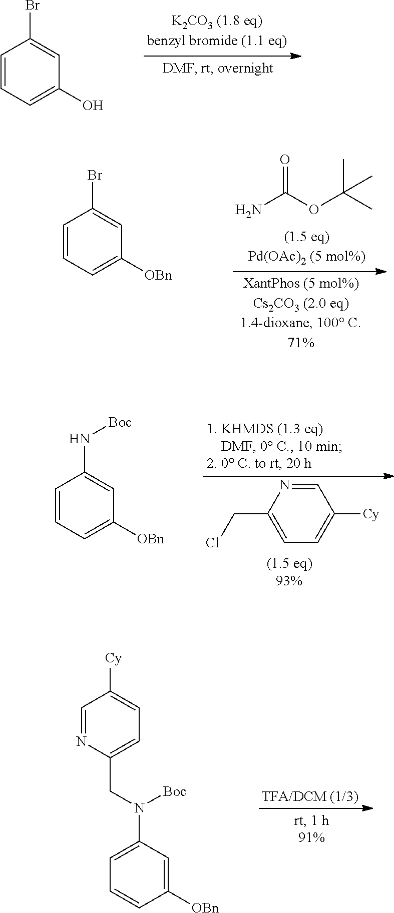

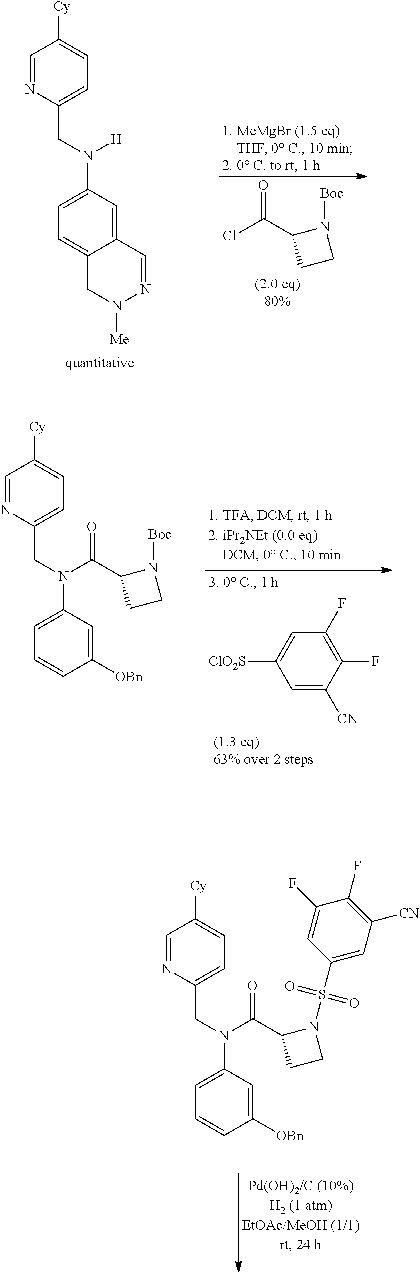

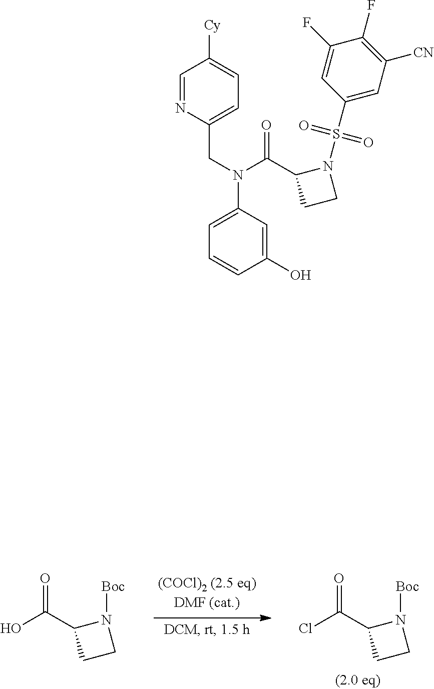

### (R)-1-((3-cyano-4,5-difluorophenyl)sulfonyl)-N-((5-cyclohexylpyridin-2-yl)methyl)-N-(3-hydroxyphenyl)azetidine-2-carboxamide

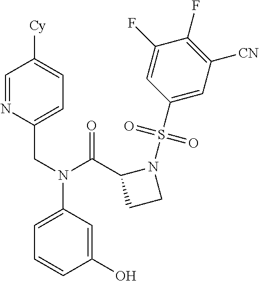

Step 1: To a solution of 3-bromophenol (2.0 g, 11.56 mmol, 1.00 equiv) and K2CO3 (2.4 g, 17.34 mmol, 1.5 equiv) in 15 mL DMF was added benzyl bromide (1.5 mL, 12.72 mmol, 1.1 equiv) under Argon. The reaction was stirred at room temperature for 24 h. Then the reaction was quenched with water, and extracted with EtOAc (3×). The combined organic extracts were washed with saturated NH4Cl aq and brine, dried over anhydrous sodium sulfate, and concentrated under reduced pressure. The residue was diluted with hexane and concentrated again under reduced pressure to obtain 1-(benzyloxy)-3-bromobenzene as white solid (3.1 g, quantitative). 1H NMR (300 MHz, CDCl3) δ 7.48-7.31 (m, 5H), 7.21-7.07 (m, 3H), 6.96-6.88 (m, 1H), 5.05 (s, 2H).

Step 2: 1-(benzyloxy)-3-bromobenzene (1.0 g, 3.80 mmol, 1.0 equiv), tert-butyl carbamate (668 mg, 5.70 mmol, 1.5 equiv), and Cs2CO3 (2.48 g, 7.60 mmol, 2.0 equiv) were dissolved in 40 mL 1,4-dioxane. After 10 min, Pd(OAc)2 (43 mg, 0.19 mmol, 5 mol %) and XantPhos (110 mg, 0.19 mmol, 5 mol %) were added to the reaction under Argon. Then the reaction was heated at 100 degrees C. for 24 h. The reaction was quenched with water, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 20/1) to provide tert-butyl (3-(benzyloxy)phenyl)carbamate as white solid (800 mg, 71%). 1H NMR (300 MHz, CDCl3) δ 7.50-7.30 (m, 5H), 7.25-7.15 (m, 2H), 6.91-6.84 (m, 1H), 6.72-6.64 (m, 1H), 6.49 (s, 1H), 5.08 (s, 2H), 1.54 (s, 9H).

Step 3: To a solution of tert-butyl (3-(benzyloxy)phenyl)carbamate (400 mg, 1.34 mmol, 1.0 equiv) in 7 mL DMF was added KHMDS (1.7 mL, 1.74 mmol, 1.3 equiv, 1.0 M in THF) at 0 degrees C. dropwise under Argon. 10 min late, 2-(chloromethyl)-5-cyclohexylpyridine (4.0 mL, 2.00 mmol, 1.5 equiv, 0.5 M in toluene) was added to the reaction mixture. The reaction was allowed to warm up to room temperature and stirred at room temperature for 20 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 10/1) to provide tert-butyl (3-(benzyloxy)phenyl)((5-cyclohexylpyridin-2-yl)methyl)carbamate as light yellow oil (680 mg, 93%). 1H NMR (300 MHz, CDCl3) δ 8.38 (d, J=2.0 Hz, 1H), 7.48 (dd, J=8.1, 2.2 Hz, 1H), 7.44-7.27 (m, 5H), 7.24 (d, J=8.0 Hz, 1H), 7.18 (t, J=8.1 Hz, 1H), 6.95 (s, 1H), 6.89 (d, J=9.0 Hz, 1H), 6.80-6.73 (m, 1H), 5.00 (s, 2H), 4.91 (s, 2H), 2.58-2.45 (m, 1H), 1.93-1.71 (m, 6H), 1.49-1.31 (m, 13H).

Step 4: To a solution of tert-butyl (3-(benzyloxy)phenyl)((5-cyclohexylpyridin-2-yl)methyl)carbamate (630 mg, 1.33 mmol) in 10 mL DCM was added TFA (5 mL). The reaction was stirred at room temperature for 1 h. Then the reaction was concentrated under reduced pressure, then quenched with saturated NaHCO3 aq. to pH 8. The reaction was extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 5/1) to provide 3-(benzyloxy)-N-((5-cyclohexylpyridin-2-yl)methyl)aniline as light yellow oil (451 mg, 91%). 1H NMR (300 MHz, CDCl3) δ 8.46 (d, J=1.9 Hz, 1H), 7.53-7.33 (m, 6H), 7.26 (d, J=8.1 Hz, 1H), 7.16-7.06 (m, 1H), 6.44-6.31 (m, 3H), 5.04 (s, 2H), 4.43 (s, 2H), 2.62-2.50 (m, 1H), 1.99-1.72 (m, 6H), 1.51-1.38 (m, 4H).

Step 5a. To a solution of (R)-1-(tert-butoxycarbonyl)azetidine-2-carboxylic acid (277 mg, 1.29 mmol, 2.0 equiv) in 10 mL DCM was added DMF (1 drop, cat.) and oxalyl chloride (0.14 mL, 1.61 mmol, 2.5 equiv) dropwise under Argon. The reaction was stirred at room temperature for 1.5 h. Then the mixture was concentrated under reduced pressure, diluted with dry DCE and concentrated again. The resulting acid chloride was dried under high vacuum for 30 min and used directly for the next step.

Step 5b. To a solution of 3-(benzyloxy)-N-((5-cyclohexylpyridin-2-yl)methyl)aniline (240 mg, 0.64 mmol, 1.0 equiv) in 6 mL THF was added MeMgBr (0.7 mL, 0.97 mmol, 1.5 equiv, 1.4 M in THF/toluene) at 0 degrees C. under Argon. 10 min later, a solution of the above acid chloride in 2 mL THF was added to the reaction. The reaction was allowed to warm up to room temperature and stirred for 1 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc/MeOH 10/1/2%) to provide tert-butyl (R)-2-((3-(benzyloxy)phenyl)((5-cyclohexylpyridin-2-yl)methyl)carbamoyl)azetidine-1-carboxylate as light yellow oil (285 mg, 80%). 1H NMR (300 MHz, CDCl3) δ 8.31 (s, 1H), 7.51-7.44 (m, 1H), 7.44-7.29 (m, 5H), 7.26-7.16 (m, 2H), 6.91 (d, J=6.8 Hz, 1H), 6.85-6.68 (m, 2H), 5.08-4.85 (m, 4H), 4.66-4.55 (m, 1H), 4.16-3.98 (m, 2H), 3.95-3.80 (m, 1H), 3.77-3.66 (m, 1H), 2.54-2.45 (m, 1H), 1.88-1.70 (m, 6H), 1.50-1.32 (m, 13H).

Step 6: To a solution of tert-butyl (R)-2-((3-(benzyloxy)phenyl)((5-cyclohexylpyridin-2-yl)methyl)carbamoyl)azetidine-1-carboxylate (268 mg, 0.48 mmol) in 5 mL DCM was added TFA (1.5 mL). the reaction was stirred at room temperature for 1 h. Then the reaction was concentrated under reduced pressure, diluted with dry DCE and concentrated again. The resulting free azetidine was dried under high vacuum for 30 min and used directly for the next step.

Step 7. To a solution of the above free azetidine in 8 mL DCM was added DIPEA (0.5 mL, 2.89 mmol, 6.0 equiv) at 0 degrees C. under Argon. After 10 min, a solution of 3-cyano-4,5-difluorobenzenesulfonyl chloride (149 mg, 0.63 mmol, 1.3 equiv) in 2 mL DCM was added dropwise under Argon at 0 degrees C. The reaction was stirred at 0 degrees C. for 1 h. Then the reaction was quenched with saturated NH4Cl aq, extracted with DCM (3×). The combined organic extracts were washed with saturated brine, dried over anhydrous sodium sulfate, and concentrated under reduced pressure. The resulting residue was purified by flash chromatography (eluent: hexane/EtOAc 1/1) to provide (R)—N-(3-(benzyloxy)phenyl)-1-((3-cyano-4,5-difluorophenyl)sulfonyl)-N-((5-cyclohexylpyridin-2-yl)methyl)azetidine-2-carboxamide as white gum (200 mg, 63% over 3 steps). 1H NMR (300 MHz, CDCl3) δ 8.35 (d, J=1.8 Hz, 1H), 8.23-8.12 (m, 1H), 8.08-7.97 (m, 1H), 7.50 (dd, J=8.0, 2.1 Hz, 1H), 7.43-7.27 (m, 5H), 7.26-7.22 (m, 2H), 6.98 (d, J=7.4 Hz, 1H), 6.80-6.67 (m, 2H), 5.11-4.88 (m, 5H), 3.99-3.85 (m, 1H), 3.69-3.55 (m, 1H), 2.57-2.44 (m, 1H), 2.30-2.17 (m, 1H), 1.94-1.66 (m, 7H), 1.49-1.33 (m, 4H).

Step 8. (R)—N-(3-(benzyloxy)phenyl)-1-((3-cyano-4,5-difluorophenyl)sulfonyl)-N-((5-cyclohexylpyridin-2-yl)methyl)azetidine-2-carboxamide (189 mg, 0.29 mmol) and Pd(OH)2/C (20 mg) were dissolved in EtOAc/MeOH (6 mL, 1/1) under hydrogen gas (1 atm). After 24 h, the catalyst was filtered off through a celite pad and washed with ethyl acetate. The combined solvent was concentrated under reduced pressure and the residue was purified by preparative TLC plates (eluent: hexane/EtOAc 1/2) to obtain (R)-1-((3-cyano-4,5-difluorophenyl)sulfonyl)-N-((5-cyclohexylpyridin-2-yl)methyl)-N-(3-hydroxyphenyl)azetidine-2-carboxamide as white solid (20 mg, 12%). 1H NMR (300 MHz, CD3OD) δ 8.29 (s, 1H), 8.06-7.97 (m, 1H), 7.97-7.91 (m, 1H), 7.72-7.64 (m, 1H), 7.40 (d, J=8.1 Hz, 1H), 7.25 (t, J=8.0 Hz, 1H), 6.84 (dd, J=8.2, 2.1 Hz, 1H), 6.66 (d, J=7.7 Hz, 1H), 6.63-6.57 (m, 1H), 5.07-4.91 (m, 2H), 4.69 (t, J=8.2 Hz, 1H), 3.82-3.70 (m, 2H), 2.63-2.52 (m, 1H), 2.46-2.34 (m, 1H), 1.98-1.69 (m, 7H), 1.50-1.39 (m, 4H). LRMS (ESI) m/z 567.2 [M+H]+; HRMS (ESI) m/z 567.1877 [M+H]+, 589.1698 [M+Na]+; Purity 97%.

### Example 11

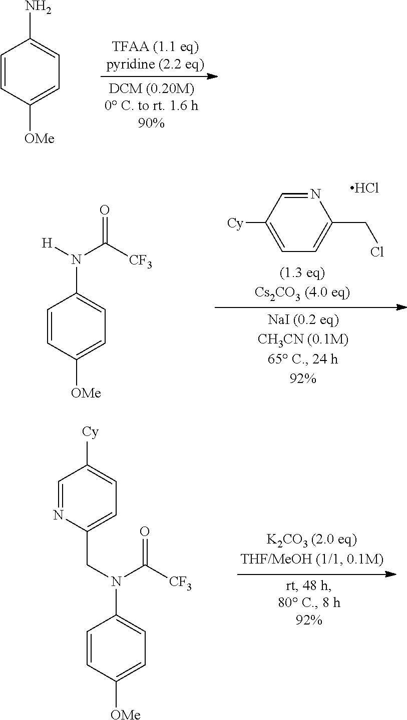

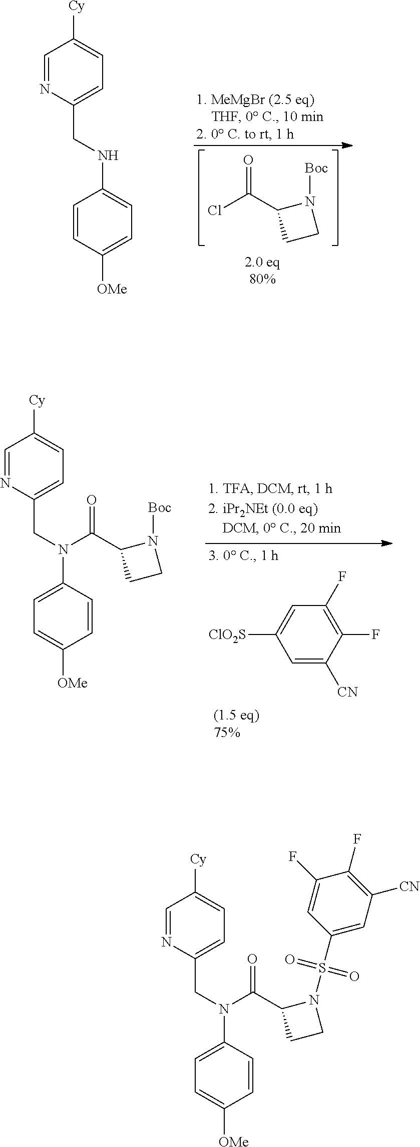

### (R)-1-((3-cyano-4,5-difluorophenyl)sulfonyl)-N-((5-cyclohexylpyridin-2-yl)methyl)-N-(4-methoxyphenyl)azetidine-2-carboxamide

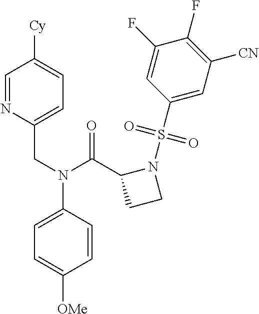

Step 1: To a solution of 4-methoxyaniline (2.0 g, 16.24 mmol, 1.00 equiv) in 60 mL DCM was added pyridine (2.8 mL, 35.73 mmol, 2.2 equiv) and TFAA (2.5 mL, 17.86 mmol, 1.1 equiv) at 0 degrees C. under Argon. The reaction was allowed to warm up to room temperature and stirred for 2 h. Then the reaction was quenched with 10% KHSO4/Na2SO4 buffer, and extracted with DCM (3×). The combined organic extracts were washed with saturated brine, dried over anhydrous sodium sulfate, and concentrated under reduced pressure. The residue was diluted with hexane and filtered to obtain the crude product as light red solid (3.2 g, 90%). 1H NMR (300 MHz, CDCl3) δ 7.86 (s, 1H), 7.47 (d, J=9.0 Hz, 2H), 6.91 (dd, J=7.0, 2.0 Hz, 2H), 3.81 (s, 3H). 19F NMR (282 MHz, CDCl3) δ−75.66.

Step 2: 2,2,2-trifluoro-N-(4-methoxyphenyl)acetamide (200 mg, 0.91 mmol, 1.0 equiv), 2-(chloromethyl)-5-cyclohexylpyridine hydrochloride (337 mg, 1.37 mmol, 1.5 equiv), Cs2CO3 (1.19 g, 3.65 mmol, 4.0 equiv), and NaI (27 mg, 0.18 mmol, 0.2 equiv) were dissolved in 10 mL MeCN under Argon. Then the reaction was heated at 65 degrees C. for 24 h. The reaction was quenched with water, extracted with ethyl acetate (3×). The combined organic extracts were washed with 10% KHSO4/Na2SO4 buffer and saturated brine, dried over anhydrous sodium sulfate, and concentrated under reduced pressure. The resulting residue was purified by flash chromatography (eluent: hexane/EtOAc 20/1) to provide N-((5-cyclohexylpyridin-2-yl)methyl)-2,2,2-trifluoro-N-(4-methoxyphenyl)acetamide as light yellow oil (329 mg, 92%).

Step 3: N-((5-cyclohexylpyridin-2-yl)methyl)-2,2,2-trifluoro-N-(4-methoxyphenyl)acetamide (446 mg, 1.14 mmol, 1.0 equiv) and K2CO3 (314 mg, 2.27 mmol, 2.0 equiv) in 5 mL THF and 5 mL MeOH. The reaction was stirred at room temperature for 48 h. Then the reaction was heated at 60 degrees C. for 8 h. The reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc/MeOH 8/1/2%) to provide N-((5-cyclohexylpyridin-2-yl)methyl)-4-methoxyaniline as light red oil (310 mg, 92%). 1H NMR (300 MHz, CDCl3) δ 8.42 (d, J=2.2 Hz, 1H), 7.47 (dd, J=8.1, 2.3 Hz, 1H), 7.27-7.24 (m, 1H), 6.82-6.73 (m, 2H), 6.68-6.60 (m, 2H), 4.37 (s, 2H), 3.74 (s, 3H), 2.58-2.47 (m, 1H), 1.92-1.72 (m, 6H), 1.46-1.35 (m, 4H).

Step 4a. To a solution of (R)-1-(tert-butoxycarbonyl)azetidine-2-carboxylic acid (407 mg, 2.02 mmol, 2.0 equiv) in 20 mL DCM was added DMF (2 drops, cat.) and oxalyl chloride (0.22 mL, 2.53 mmol, 2.5 equiv) dropwise under Argon. The reaction was stirred at room temperature for 1.5 h. Then the mixture was concentrated under reduced pressure, diluted with dry DCE and concentrated again. The resulting acid chloride was dried under high vacuum for 30 min and used directly for the next step.

Step 4b. To a solution of N-((5-cyclohexylpyridin-2-yl)methyl)-4-methoxyaniline (300 mg, 1.01 mmol, 1.0 equiv) in 6 mL THF was added MeMgBr (1.8 mL, 2.53 mmol, 2.5 equiv, 1.4 M in THF/toluene) at 0 degrees C. under Argon. 10 min later, a solution of the above acid chloride in 4 mL THF was added to the reaction. The reaction was allowed to warm up to room temperature and stirred for 1 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc/MeOH 10/1/2%) to provide tert-butyl (R)-2-(((5-cyclohexylpyridin-2-yl)methyl)(4-methoxyphenyl)carbamoyl)azetidine-1-carboxylate as yellow oil (386 mg, 80%). 1H NMR (300 MHz, CDCl3) δ 8.30 (s, 1H), 7.50 (dd, J=8.1, 2.1 Hz, 1H), 7.43 (d, J=8.0 Hz, 1H), 7.01 (s, 2H), 6.82 (d, J=8.6 Hz, 2H), 5.12-4.98 (m, 1H), 4.93 (s, 1H), 4.57 (s, 1H), 4.08-3.99 (m, 1H), 3.82-3.68 (m, 1H), 3.78 (s, 3H), 2.48 (m, 1H), 2.19-2.07 (m, 2H), 1.79 (m, 6H), 1.49-1.30 (m, 13H).

Step 5a. To a solution of tert-butyl (R)-2-(((5-cyclohexylpyridin-2-yl)methyl)(4-methoxyphenyl)carbamoyl)azetidine-1-carboxylate (365 mg, 0.76 mmol, 1.0 equiv) in 10 mL DCM was added TFA (3.5 mL). the reaction was stirred at room temperature for 1 h. Then the reaction was concentrated under reduced pressure, diluted with dry DCE and concentrated again. The resulting crude free azetidine was dried under high vacuum for 30 min and used directly for the next step.

Step 5b. To a solution of the above free azetidine in 8 mL DCM was added DIPEA (0.76 mL, 4.57 mmol, 6.0 equiv) at 0 degrees C. under Argon. After 10 min, a solution of 3-cyano-4,5-difluorobenzenesulfonyl chloride (271 mg, 1.14 mmol, 1.5 equiv) in 2 mL DCM was added dropwise under Argon at 0 degrees C. The reaction was stirred at 0 degrees C. for 1 h. Then the reaction was quenched with saturated NH4Cl aq, extracted with DCM (3×). The combined organic extracts were washed with saturated brine, dried over anhydrous sodium sulfate, and concentrated under reduced pressure. The resulting residue was purified by flash chromatography (eluent: hexane/EtOAc/MeOH 10/1/1%) to provide (R)-1-((3-cyano-4,5-difluorophenyl)sulfonyl)-N-((5-cyclohexylpyridin-2-yl)methyl)-N-(4-methoxyphenyl)azetidine-2-carboxamide as light yellow solid (330 mg, 75% over 3 steps). 1H NMR (300 MHz, CDCl3) δ 8.37-8.32 (m, 1H), 8.25-8.16 (m, 1H), 8.10-8.03 (m, 1H), 7.54-7.46 (m, 1H), 7.09-7.00 (m, 2H), 6.90-6.81 (m, 2H), 5.02-4.96 (m, 1H), 4.93 (s, 2H), 3.97 (dd, J=15.9, 8.1 Hz, 1H), 3.81 (s, 3H), 3.69-3.60 (m, 1H), 2.57-2.45 (m, 1H), 2.38-2.29 (m, 1H), 1.92-1.71 (m, 7H), 1.45-1.34 (m, 4H). 19F NMR (282 MHz, CDCl3) δ−123.02, −129.97. LRMS (ESI) m/z 581.2 [M+H]+; HRMS (ESI) m/z 581.2034 [M+H]+, 603.1854 [M+Na]+; Purity 100%.

### Example 12

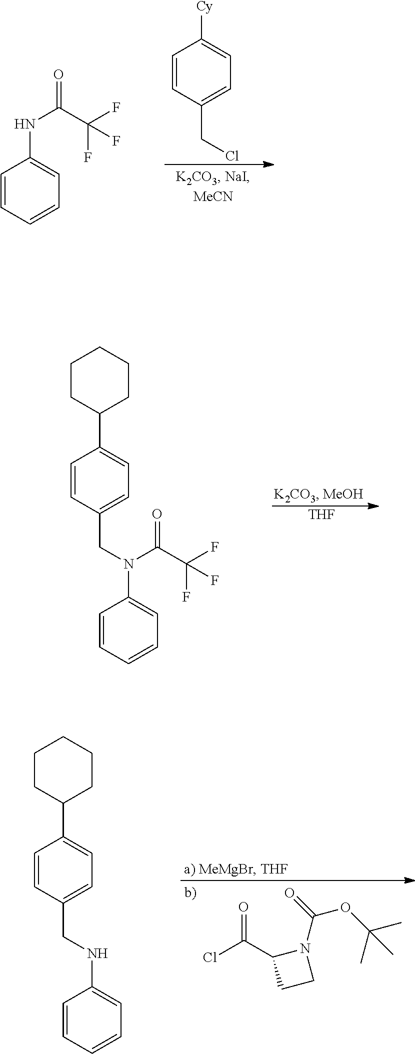

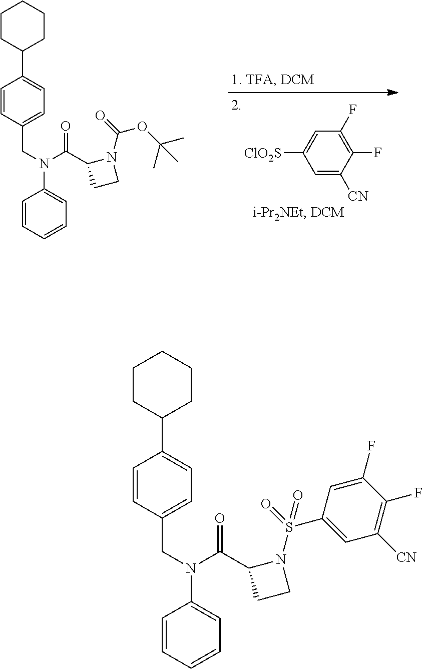 [SMILES: CC(C)(C)OC(=O)N1CC[C@@H]1C(=O)N(Cc1ccc(C2CCCCC2)cc1)c1ccccc1 ; N#Cc1cc(S(=O)(=O)Cl)cc(F)c1F ; N#Cc1cc(S(=O)(=O)N2CC[C@@H]2C(=O)N(Cc2ccc(C3CCCCC3)cc2)c2ccccc2)cc(F)c1F]

### (R)-1-((3-cyano-4,5-difluorophenyl)sulfonyl)-N-(4-cyclohexylbenzyl)-N-phenylazetidine-2-carboxamide

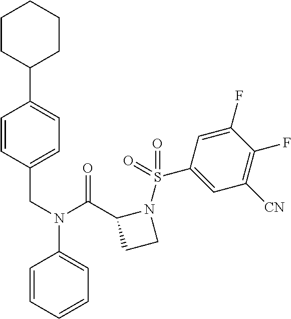 [SMILES: N#Cc1cc(S(=O)(=O)N2CC[C@@H]2C(=O)N(Cc2ccc(C3CCCCC3)cc2)c2ccccc2)cc(F)c1F]

Step 1. 2,2,2-trifluoro-N-phenylacetamide (500 mg, 2.64 mmol, 1.0 equiv), 1-(bromomethyl)-4-cyclohexylbenzene (802 mg, 3.17 mmol, 1.2 equiv), and K2CO3 (475 mg, 3.44 mmol, 1.3 equiv) were dissolved in 12 mL MeCN under Argon. Then the reaction was heated at 60 degrees C. for 3.5 h. The reaction was quenched with water, extracted with ethyl acetate (3×). The combined organic extracts were washed with aqueous 10% KHSO4/Na2SO4 buffer and saturated brine, dried over anhydrous sodium sulfate, and concentrated under reduced pressure. The resulting residue, N-(4-cyclohexylbenzyl)-2,2,2-trifluoro-N-phenylacetamide, was used direct for the next step.

Step 2. The above residue, N-(4-cyclohexylbenzyl)-2,2,2-trifluoro-N-phenylacetamide, and K2CO3 (730 mg, 5.28 mmol, 2.0 equiv) were dissolved in 10 mL THF and 10 mL MeOH. The reaction was stirred at room temperature for 2 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 200/1) to provide N-(4-cyclohexylbenzyl)aniline as colorless oil (560 mg, 80%). 1H NMR (300 MHz, CDCl3) δ 7.35-7.28 (m, 2H), 7.26-7.17 (m, 4H), 6.78-6.71 (m, 1H), 6.71-6.64 (m, 2H), 4.31 (s, 2H), 2.59-2.46 (m, 1H), 1.97-1.73 (m, 6H), 1.49-1.37 (m, 4H).

Step 3. To N-(4-cyclohexylbenzyl)aniline (255 mg, 0.96 mmol) in THF (7 mL) under Ar was added MeMgBr (1.4 M in THF, 2.5 eq) at 0 degrees C. The solution was stirred for fifteen minutes. Then tert-butyl (R)-2-(chlorocarbonyl)azetidine-1-carboxylate (1.92 mmol) in a solution of THF (7 mL) was added to the reaction mixture at 0 degrees C. and allowed to warm up to room temperature. This mixture was stirred at room temperature for one hour. Saturated ammonium chloride was added to the mixture, which was then extracted (×2) with EtOAc. The organic layers were combined and washed with brine, then dried over sodium sulfate. The dried organic layers were then concentrated. Column chromatography (20% EtOAc, 80% hexanes) afforded tert-butyl (R)-2-((4-cyclohexylbenzyl)(phenyl)carbamoyl)azetidine-1-carboxylate in 25% yield. 1H NMR (300 MHz, Chloroform-d) δ 7.33 (dt, J=6.7, 2.8 Hz, 3H), 7.22-6.82 (m, 6H), 5.21-4.31 (m, 1H), 4.23-3.82 (m, 2H), 3.84-3.56 (m, 2H), 2.58-2.36 (m, 1H), 2.24-1.94 (m, 2H), 1.93-1.64 (m, 5H), 1.54-1.16 (m, 14H).

Step 4. To tert-butyl (R)-2-((4-cyclohexylbenzyl)(phenyl)carbamoyl)azetidine-1-carboxylate (108 mg, 0.23 mmol) in DCM (5 ml) was added TFA (1.1 mL) under Ar, this reaction mixture was allowed to stir at room temperature for 2 hours. The reaction mixture was concentrated, re-dissolved in DCM (10 mL), and concentrated once more. This process was repeated and the crude oil was placed onto high vacuum for forty five minutes before being used directly in the following reaction.

Step 5. To crude (R)—N-(4-cyclohexylbenzyl)-N-phenylazetidine-2-carboxamide in a solution of DCM (5 mL) under Ar at 0 degrees C. was added DIPEA (6 eq) and stirred for fifteen minutes. To this mixture was added 3-cyano-4,5-difluorobenzenesulfonyl chloride (74.4 mg, 1.3 eq) in DCM (5 mL) at 0 degrees C. The reaction mixture was then allowed to warm to room temperature and stirred for two and a half hours Ammonium chloride was added and the mixture was extracted with EtOAc (×3). The combined organic extracts were washed with brine, dried, and concentrated. Purification by column chromatography (40% EtOAc, 60% hexanes) yielded (R)-1-((3-cyano-4,5-difluorophenyl)sulfonyl)-N-(4-cyclohexylbenzyl)-N-phenylazetidine-2-carboxamide in 41% yield over two steps. 1H NMR (300 MHz, Chloroform-d) δ 8.21 (ddd, J=9.1, 7.0, 2.2 Hz, 1H), 8.06 (dt, J=5.0, 1.9 Hz, 1H), 7.39 (m, 3H), 7.17-7.06 (m, 4H), 6.99 (m, 2H), 4.92-4.82 (m, 3H), 3.96 (m, 1H), 3.67 (m, 1H), 2.49 (m, 1H), 2.30 (m, 1H), 1.93-1.69 (m, 6H), 1.49-1.20 (m, 5H). HRMS (ESI+) m/z 550.1963 [M+H]+.

### Example 13

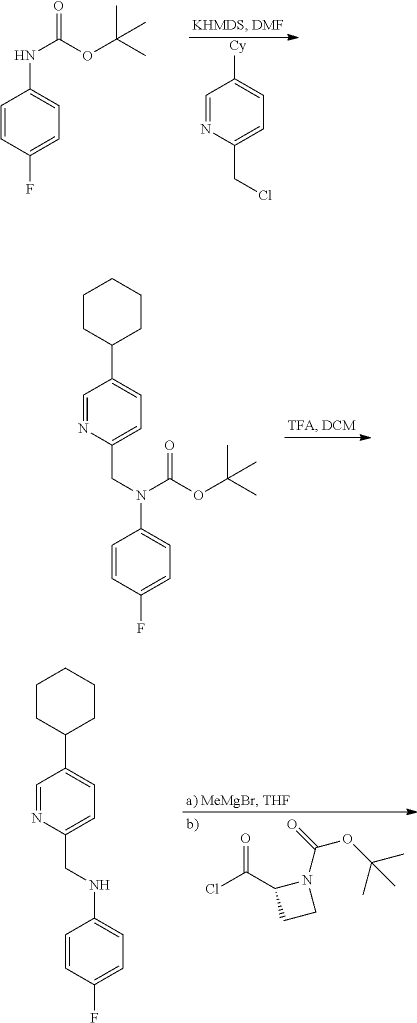

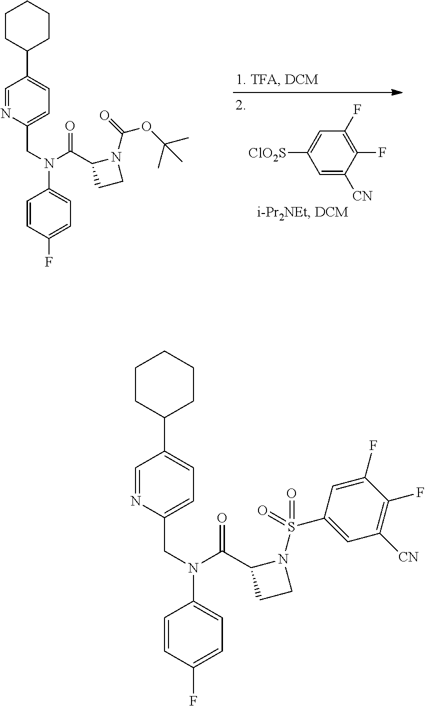 [SMILES: CC(C)(C)OC(=O)N1CC[C@@H]1C(=O)N(Cc1ccc(C2CCCCC2)cn1)c1ccc(F)cc1 ; N#Cc1cc(S(=O)(=O)Cl)cc(F)c1F ; N#Cc1cc(S(=O)(=O)N2CC[C@@H]2C(=O)N(Cc2ccc(C3CCCCC3)cn2)c2ccc(F)cc2)cc(F)c1F]

### (R)-1-((3-cyano-4,5-difluorophenyl)sulfonyl)-N-((5-cyclohexylpyridin-2-yl)methyl)-N-(4-fluorophenyl)azetidine-2-carboxamide

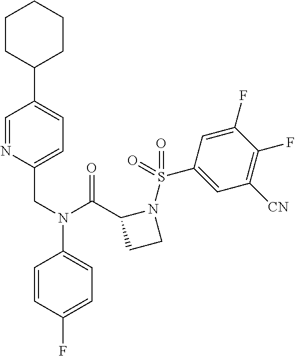 [SMILES: N#Cc1cc(S(=O)(=O)N2CC[C@@H]2C(=O)N(Cc2ccc(C3CCCCC3)cn2)c2ccc(F)cc2)cc(F)c1F]

Step 1. To a solution of tert-butyl (4-fluorophenyl)carbamate (500 mg, 2.37 mmol, 1.0 equiv) in 8 mL DMF was added KHMDS (5.4 mL, 5.40 mmol, 2.3 equiv, 1.0 M in THF) at 0 degrees C. dropwise under Argon. After 10 min, 2-(chloromethyl)-5-cyclohexylpyridine hydrochloride (758 mg, 3.07 mmol, 1.3 equiv) was added to the reaction mixture. The reaction was allowed to warm up to room temperature and stirred at room temperature for 20 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 20/1) to provide tert-butyl ((5-cyclohexylpyridin-2-yl)methyl)(4-fluorophenyl)carbamate as light yellow solid (370 mg, 41%). 1H NMR (300 MHz, CDCl3) δ 8.39 (s, 1H), 7.55-7.48 (m, 1H), 7.29-7.19 (m, 3H), 7.03-6.93 (m, 2H), 4.90 (s, 2H), 2.61-2.48 (m, 1H), 1.97-1.73 (m, 6H), 1.51-1.37 (m, 13H).

Step 2. Preparation by a similar procedure to Example 10, step 3, starting from tert-butyl ((5-cyclohexylpyridin-2-yl)methyl)(4-fluorophenyl)carbamate to obtain crude N-((5-cyclohexylpyridin-2-yl)methyl)-4-fluoroaniline, which taken as such to next step.

Step 3. To crude N-((5-cyclohexylpyridin-2-yl)methyl)-4-fluoroaniline (223 mg, 0.78 mol) in THF (6 mL) under Ar was added at 0 degrees C. MeMgBr (1.4 M in THF, 2.4 eq) and stirred for fifteen minutes. Following this tert-butyl (R)-2-(chlorocarbonyl)azetidine-1-carboxylate (1.56 mmol) dissolved in THF (6 mL) was added to the reaction mixture. The mixture was then allowed to reach room temperature and stirred for one hour. The reaction was quenched with ammonium chloride and then extracted with EtOAc (×3). The combined organic extracts were washed with brine, dried, and concentrated. Purification by column chromatography (20% EtoAc, 80% hexanes) yielded tert-butyl (R)-2-(((5-cyclohexylpyridin-2-yl)methyl)(4-fluorophenyl)carbamoyl)azetidine-1-carboxylate in 32% yield. 1H NMR (300 MHz, Chloroform-d) δ 8.30 (m, 1H), 7.51-6.91 (m, 6H), 4.54 (m, 1H), 4.21-3.66 (m, 4H), 2.46 (m, 2H), 2.17-1.70 (m, 8H), 1.46-1.18 (m, 12H). 19F NMR (282 MHz, Chloroform-d) δ−112.46-−113.27 (m).

Step 4. To tert-butyl (R)-2-(((5-cyclohexylpyridin-2-yl)methyl)(4-fluorophenyl)carbamoyl)azetidine-1-carboxylat e (118 mg, 0.25 mmol) in DCM (5 mL) under Ar was added TFA (3 eq). The reaction was allowed to stir at room temperature for two hours. The reaction mixture was concentrated, re-dissolved in DCM (10 mL), and concentrated once more. This process was repeated and the crude oil was placed onto high vacuum for forty five minutes before being used directly in the following reaction.

Step 5. To crude (R)—N-((5-cyclohexylpyridin-2-yl)methyl)-N-(4-fluorophenyl)azetidine-2-carboxamide from the previous reaction dissolved in DCM (5 ml) was added DIPEA (6 eq) at 0 degrees C. under Ar. The reaction mixture was allowed to stir for fifteen minutes. To this mixture was added 3-cyano-4,5-difluorobenzenesulfonyl chloride (77 mg, 1.3 eq) in DCM (5 mL) at 0 degrees C. The reaction mixture was then allowed to warm to room temperature and stirred for two and a half hours. Ammonium chloride was added and the mixture was extracted with EtOAc (×3). The combined organic extracts were washed with brine, dried, and concentrated. Purification by column chromatography (100% hexanes, 10% EtOAc, 20% EtOAc, 50% EtOAc) yielded (R)-1-((3-cyano-4,5-difluorophenyl)sulfonyl)-N-((5-cyclohexylpyridin-2-yl)methyl)-N-(4-fluorophenyl)azetidine-2-carboxamide in 31% yield over two steps. 1H NMR (300 MHz, Chloroform-d) δ 8.37 (d, J=2.3 Hz, 1H), 8.21 (ddd, J=9.1, 7.0, 2.2 Hz, 1H), 8.10 (dt, J=4.9, 1.9 Hz, 1H), 7.51 (dd, J=8.0, 2.3 Hz, 1H), 7.27-7.16 (m, 3H), 7.11-7.00 (m, 2H), 4.97 (m, 3H), 4.02 (dt, J=8.8, 7.4 Hz, 1H), 3.66 (m, 1H), 2.51 (m, 1H), 2.34 (m, 1H), 1.98-1.69 (m, 6H), 1.53-1.19 (m, 5H). 19F NMR (282 MHz, Chloroform-d) δ−72.84, −111.48 (tt, J=8.0, 4.8 Hz), −122.97 (ddd, J=19.8, 7.0, 4.9 Hz), −129.99 (ddd, J=19.8., 9.0, 1.7 Hz). HRMS (ESI+) m/z 569.182[M+H]+.

### Example 14

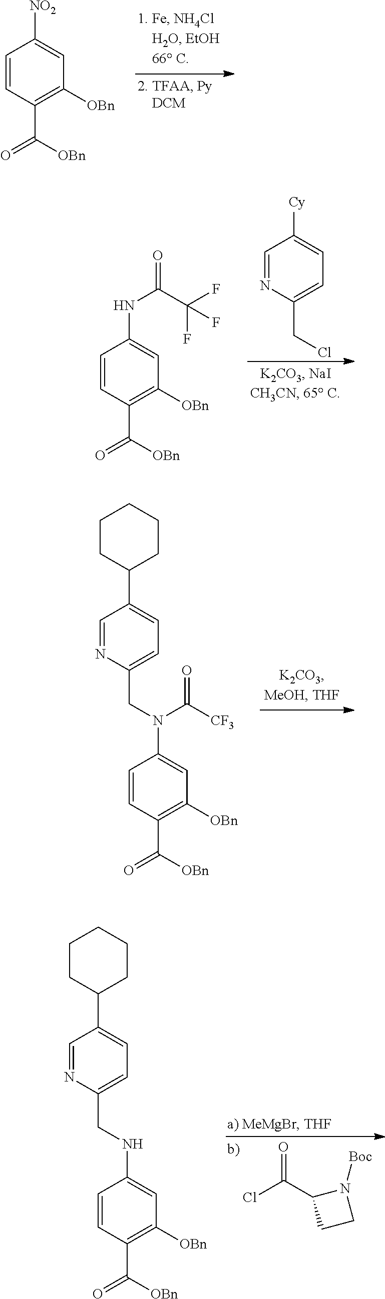

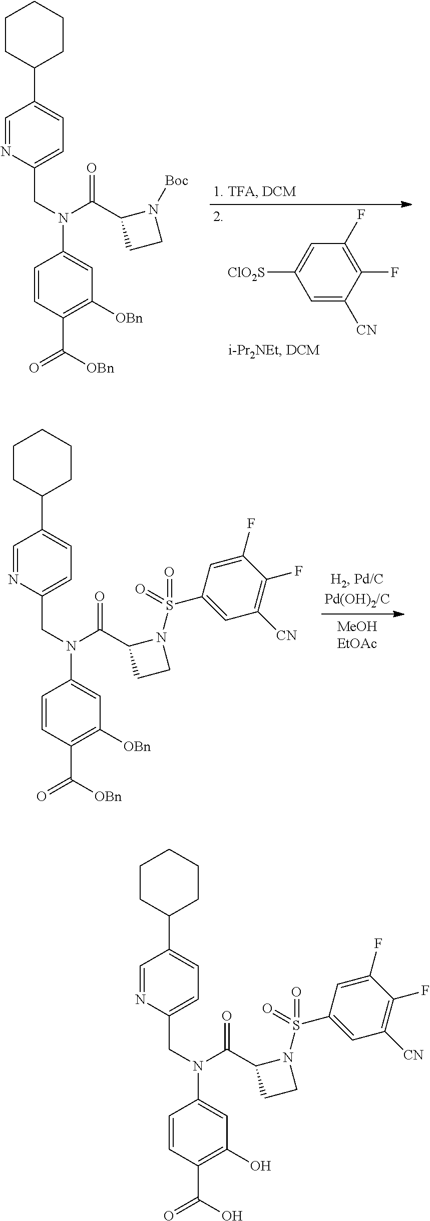 [SMILES: CC(C)(C)OC(=O)N1CC[C@@H]1C(=O)N(Cc1ccc(C2CCCCC2)cn1)c1ccc(C(=O)OCc2ccccc2)c(OCc2ccccc2)c1 ; N#Cc1cc(S(=O)(=O)Cl)cc(F)c1F ; N#Cc1cc(S(=O)(=O)N2CC[C@@H]2C(=O)N(Cc2ccc(C3CCCCC3)cn2)c2ccc(C(=O)OCc3ccccc3)c(OCc3ccccc3)c2)cc(F)c1F ; N#Cc1cc(S(=O)(=O)N2CC[C@@H]2C(=O)N(Cc2ccc(C3CCCCC3)cn2)c2ccc(C(=O)O)c(O)c2)cc(F)c1F]

### (R)-4-(1-((3-cyano-4,5-difluorophenyl)sulfonyl)-N-((5-cyclohexylpyridin-2-yl)methyl)azetidine-2-carboxamido)-2-hydroxybenzoic acid

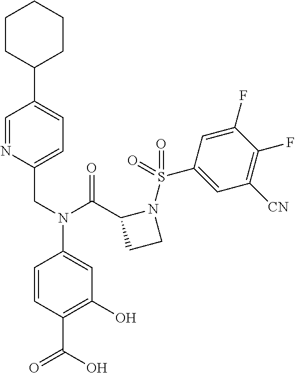 [SMILES: N#Cc1cc(S(=O)(=O)N2CC[C@@H]2C(=O)N(Cc2ccc(C3CCCCC3)cn2)c2ccc(C(=O)O)c(O)c2)cc(F)c1F]

Step 1. To benzyl 2-(benzyloxy)-4-nitrobenzoate (5.9 mmol) and ammonium chloride (60.4 mmol) were added ethanol (22 mL) and HPLC water (11 mL) under nitrogen. Iron powder (2.32 g, 41.5 at Eq) was added, and the mixture was stirred vigorously at 66 degrees C. overnight. After cooling, the mixture was filtered through celite. The cake was washed with EtOAc. Water was added to the filtrate, which was extracted with EtOAc (2×). The extract was washed with brine, dried (sodium sulfate) and concentrated to dryness to obtain benzyl 4-amino-2-(benzyloxy)benzoate. 1H NMR (500 MHz, Chloroform-d) δ 7.82 (d, J=8.3 Hz, 1H), 7.45-7.44 (m, 2H), 7.39-7.37 (m, 2H), 7.34-7.28 (m, 6H), 6.39-6.36 (m, 2H), 5.31 (s, 2H), 5.11 (s, 2H)

Step 2. Preparation by a similar procedure to Example 11, step 1, starting from benzyl 4-amino-2-(benzyloxy)benzoate to obtain benzyl 2-(benzyloxy)-4-(2,2,2-trifluoroacetamido)benzoate. 1H NMR (500 MHz, Chloroform-d) δ 8.22 (s, 1H), 7.90 (d, J=8.5 Hz, 1H), 7.62 (d, J=2.0 Hz, 1H), 7.46-7.28 (m, 10H), 7.00 (dd, J=8.5, 2.0 Hz, 1H), 5.34 (s, 2H), 5.15 (s, 2H).

Step 3. Preparation by a similar procedure to Example 5, step 2, starting from benzyl 2-(benzyloxy)-4-(2,2,2-trifluoroacetamido)benzoate to obtain benzyl 2-(benzyloxy)-4-(N-((5-cyclohexylpyridin-2-yl)methyl)-2,2,2-trifluoroacetamido)benzoate 1H NMR (300 MHz, Chloroform-d) δ 8.38 (d, J=2.3 Hz, 1H), 7.83 (d, J=8.2 Hz, 1H), 7.48 (dd, J=8.0, 2.3 Hz, 1H), 7.42-7.28 (m, 10H), 7.17 (d, J=8.0 Hz, 1H), 6.93 (d, J=1.9 Hz, 1H), 6.87 (dd, J=8.3, 1.8 Hz, 1H), 5.33 (s, 2H), 5.02 (s, 2H), 4.97 (s, 2H), 2.59-2.43 (m, 1H), 1.92-1.69 (m, 5H), 1.50-1.18 (m, 5H).

Step 4. Preparation by a similar procedure to Example 5, step 3, starting from benzyl 2-(benzyloxy)-4-(N-((5-cyclohexylpyridin-2-yl)methyl)-2,2,2-trifluoroacetamido)benzoate to obtain benzyl 2-(benzyloxy)-4-(((5-cyclohexylpyridin-2-yl)methyl)amino)benzoate 1H NMR (300 MHz, Chloroform-d) δ 8.43 (d, J=2.2 Hz, 1H), 7.85 (d, J=8.5 Hz, 1H), 7.57-7.27 (m, 11H), 7.18 (d, J=8.0 Hz, 1H), 6.34-6.16 (m, 2H), 5.38-5.27 (m, 3H), 5.11 (s, 2H), 4.42 (d, J=4.5 Hz, 2H), 2.61-2.46 (m, 1H), 1.95-1.70 (m, 5H), 1.51-1.20 (m, 5H).

Step 5. Preparation by a similar procedure to Example 2, step 5, starting from benzyl 2-(benzyloxy)-4-(((5-cyclohexylpyridin-2-yl)methyl)amino)benzoate to obtain tert-butyl (R)-2-((3-(benzyloxy)-4-((benzyloxy)carbonyl)phenyl)((5-cyclohexylpyridin-2-yl)methyl)carbamoyl)azetidine-1-carboxylate 1H NMR (300 MHz, Chloroform-d) δ 8.31 (d, J=2.0 Hz, 1H), 7.80 (d, J=8.3 Hz, 1H), 7.46 (dd, J=8.0, 2.3 Hz, 1H), 7.41-7.28 (m, 10H), 7.19-7.05 (m, 1H), 6.95-6.71 (m, 2H), 5.33 (s, 2H), 5.18-4.87 (m, 4H), 4.09-3.96 (m, 1H), 3.78-3.63 (m, 1H), 2.57-2.40 (m, 1H), 2.14-2.00 (m, 1H), 2.00-1.90 (m, 1H), 1.88-1.74 (m, 5H), 1.55-1.18 (m, 14H).

Step 6. Preparation by a similar procedure to Example 1, step 6, starting from tert-butyl (R)-2-((3-(benzyloxy)-4-((benzyloxy)carbonyl)phenyl)((5-cyclohexylpyridin-2-yl)methyl)carbamoyl)azetidine-1-carboxylate to obtain crude benzyl (R)-2-(benzyloxy)-4-(N-((5-cyclohexylpyridin-2-yl)methyl)azetidine-2-carboxamido)benzoat e, which was taken to next step.

Step 7. Preparation by a similar procedure to Example 1, step 7, starting from crude benzyl (R)-2-(benzyloxy)-4-(N-((5-cyclohexylpyridin-2-yl)methyl)azetidine-2-carboxamido)benzoate to obtain benzyl (R)-2-(benzyloxy)-4-(1-((3-cyano-4,5-difluorophenyl)sulfonyl)-N-((5-cyclohexylpyridin-2-yl)methyl)azetidine-2-carboxamido)benzoate 1H NMR (300 MHz, Chloroform-d) δ 8.36 (d, J=2.3 Hz, 1H), 8.21-8.11 (m, 1H), 8.09-8.03 (m, 1H), 7.82 (d, J=8.2 Hz, 1H), 7.48 (dd, J=8.0, 2.3 Hz, 1H), 7.43-7.28 (m, 10H), 7.19 (d, J=8.4 Hz, 1H), 6.88 (d, J=1.9 Hz, 1H), 6.80 (dd, J=8.2, 1.9 Hz, 1H), 5.42-4.74 (m, 7H), 3.99-3.83 (m, 1H), 3.63-3.44 (m, 1H), 2.57-2.44 (m, 1H), 2.15-1.99 (m, 1H), 1.95-1.66 (m, 6H), 1.50-1.14 (m, 5H).

Step 8. To a round-bottom flask equipped with a stir bar was added (R)-benzyl 2-(benzyloxy)-4-(1-cyano-4,5-difluorophenyl)sulfonyl)-N-((5-cyclohexylpyridin-2-yl)methyl)azetidine-2-carboxamido)benzoate (0.122 mmol) followed by 15% by weight 1:1 10% Pd/C: 20% Pd(OH)2/C (0.015 g each) and 1:1 ethyl acetate/methanol (2.2 mL). The mixture was exchanged with hydrogen gas three times before stirring at 25 degrees C. After 5 hours an additional 10% by weight Pd/C: Pd(OH)2/C (0.006 g each) was added and the flask was exchanged with hydrogen gas three times before being allowed to stir at 25□C. After stirring for 18 hours at 25 degrees C. the reaction was complete. The mixture was flushed through a celite plug with ethyl acetate containing 10% methanol. The filtrate was concentrated and the crude material was purified via prep-TLC. This process gave (R)-4-(1-((3-cyano-4,5-difluorophenyl)sulfonyl)-N-((5-cyclohexylpyridin-2-yl)methyl)azetidine-2-carboxamido)-2-hydroxybenzoic acid (50% yield) as a white solid. 1H NMR (300 MHz, DMSO-d6) δ 8.34 (d, J=2.2 Hz, 1H), 8.29-8.20 (m, 1H), 8.18-8.04 (m, 1H), 7.73-7.51 (m, 2H), 7.24 (d, J=8.1 Hz, 1H), 6.60-6.39 (m, 2H), 4.99-4.49 (m, 3H), 3.87-3.56 (m, 2H), 2.36-2.18 (m, 1H), 1.98-1.57 (m, 6H), 1.52-1.16 (m, 5H). HPLC purity: 100%, HRMS (ESI) m/z=611.1768 [M+H]+, HRMS (ESI+) calculated for C30H28F2N4O6S: 610.16976, found 610.16954.

### Example 15

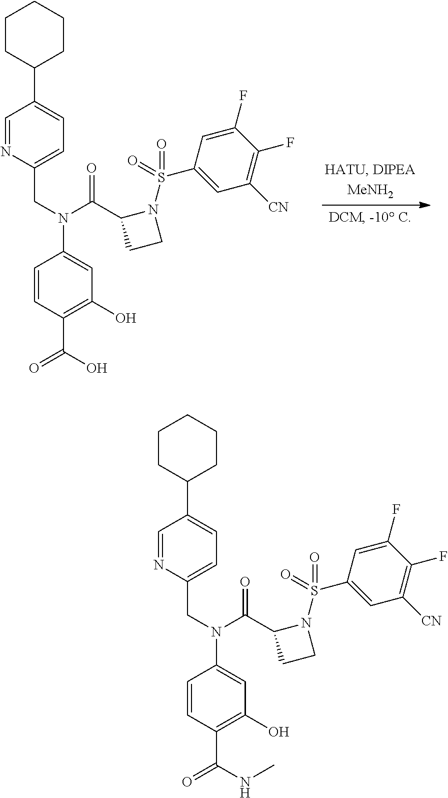 [SMILES: N#Cc1cc(S(=O)(=O)N2CC[C@@H]2C(=O)N(Cc2ccc(C3CCCCC3)cn2)c2ccc(C(=O)O)c(O)c2)cc(F)c1F ; CNC(=O)c1ccc(N(Cc2ccc(C3CCCCC3)cn2)C(=O)[C@H]2CCN2S(=O)(=O)c2cc(F)c(F)c(C#N)c2)cc1O]

### (R)-1-((3-cyano-4,5-difluorophenyl)sulfonyl)-N-((5-cyclohexylpyridin-2-yl)methyl)-N-(3-hydroxy-4-(methylcarbamoyl)phenyl)azetidine-2-carboxamide

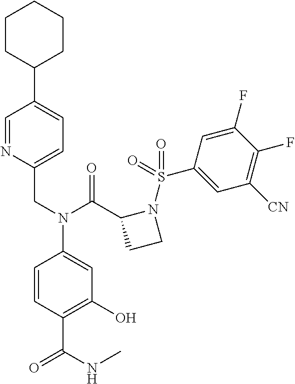 [SMILES: CNC(=O)c1ccc(N(Cc2ccc(C3CCCCC3)cn2)C(=O)[C@H]2CCN2S(=O)(=O)c2cc(F)c(F)c(C#N)c2)cc1O]

Step 1. To a round-bottom flask equipped with a stir bar was added (R)-benzyl 2-(benzyloxy)-4-(1-((3-cyano-4,5-difluorophenyl)sulfonyl)-N-((5-cyclohexylpyridin-2-yl)methyl)azetidine-2-carboxamido)benzoate (0.083 mmol) in DCM (2.3 mL). The mixture was allowed to stir at 0 degrees C. for 2 minutes before DIPEA (0.083 mmol) and HATU (0.083 mmol) were added. This solution was allowed to stir for 1.5 hours at 25 degrees C. before being cooled to −10 degrees C. After stirring for 2 minutes at −10 degrees C., methylamine (0.072 mmol) and DIPEA (0.083 mmol) were added and the solution was allowed to stir at −100 C. After stirring for 3 hours and 45 minutes at −10 degrees C. the reaction was complete. The reaction mixture was quenched with Sat. NH4Cl, extracted with DCM, dried with Na2SO4, and concentrated in vacuo. The crude product was purified via prep-TLC yielding (R)-1-((3-cyano-4,5-difluorophenyl)sulfonyl)-N-((5-cyclohexylpyridin-2-yl)methyl)-N-(3-hydroxy-4-(methylcarbamoyl)phenyl)azetidine-2-carboxamide (9% yield) as a white powder. 1H NMR (300 MHz, Chloroform-d) δ 12.64 (s, 1H), 8.34 (d, J=3.0 Hz, 1H), 8.18-8.06 (m, 1H), 8.04-7.94 (m, 1H), 7.50 (dd, J=8.1, 2.2 Hz, 1H), 7.41 (d, J=8.9 Hz, 1H), 7.24 (d, J=9.0 Hz, 1H), 6.80-6.69 (m, 2H), 6.61-6.52 (m, 1H), 5.09-4.81 (m, 3H), 4.03-3.81 (m, 1H), 3.78-3.59 (m, 1H), 3.01 (d, J=4.8 Hz, 3H), 2.56-2.44 (m, 1H), 2.43-2.29 (m, 1H), 2.08-1.92 (m, 1H), 1.91-1.58 (m, 5H), 1.52-1.28 (m, 5H). HPLC purity: 100%, HRMS (ESI) m/z=646.1933 [M+Na]+, HRMS (ESI+) calculated for C31H31F2N5O5S: 623.20408, found 623.2014.

### Example 16

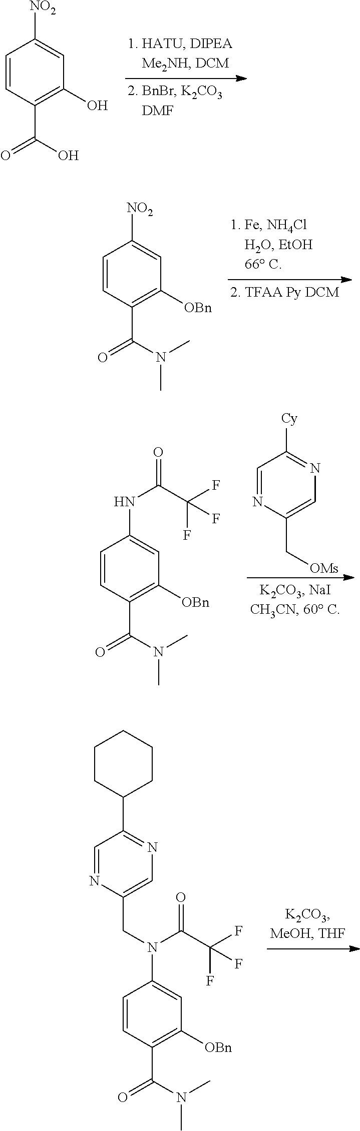

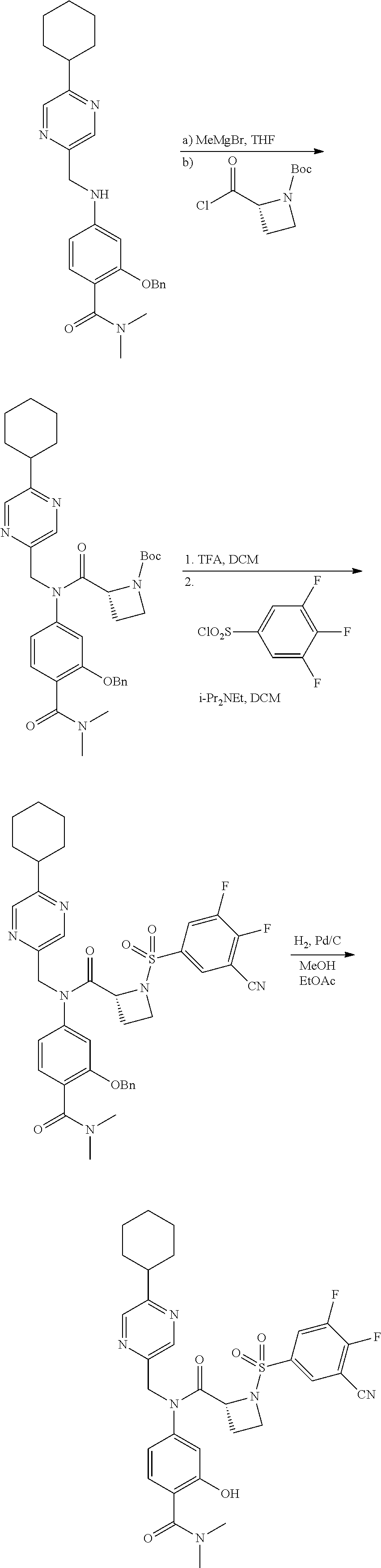 [SMILES: CN(C)C(=O)c1ccc(NCc2cnc(C3CCCCC3)cn2)cc1OCc1ccccc1 ; CC(C)(C)OC(=O)N1CC[C@@H]1C(=O)Cl ; CN(C)C(=O)c1ccc(N(Cc2cnc(C3CCCCC3)cn2)C(=O)[C@H]2CCN2C(=O)OC(C)(C)C)cc1OCc1ccccc1 ; O=S(=O)(Cl)c1cc(F)c(F)c(F)c1 ; CN(C)C(=O)c1ccc(N(Cc2cnc(C3CCCCC3)cn2)C(=O)[C@H]2CCN2S(=O)(=O)c2cc(F)c(F)c(C#N)c2)cc1OCc1ccccc1 ; CN(C)C(=O)c1ccc(N(Cc2cnc(C3CCCCC3)cn2)C(=O)[C@H]2CCN2S(=O)(=O)c2cc(F)c(F)c(C#N)c2)cc1O]

### (R)-1-((3-cyano-4,5-difluorophenyl)sulfonyl)-N-((5-cyclohexylpyrazin-2-yl)methyl)-N-(4-(dimethylcarbamoyl)-3-hydroxyphenyl)azetidine-2-carboxamide

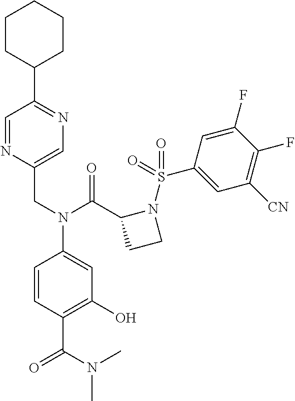 [SMILES: CN(C)C(=O)c1ccc(N(Cc2cnc(C3CCCCC3)cn2)C(=O)[C@H]2CCN2S(=O)(=O)c2cc(F)c(F)c(C#N)c2)cc1O]

Step 1. To a solution of 105 mg of 2-hydroxy-4-nitrobenzoic acid in 6 ml DCM at 0 degrees C., 0.9 eq. DIPEA and 1.0 eq. HATU were added. The mixture was allowed to warm to room temperature, and stirring for 1.5 h, then 1.5 eq. dimethyl amine was added, and the mixture was stirred overnight. After the reaction was completed, 5 ml H2O was added. The mixture was extracted with DCM 2 times, and the organic layer was washed with brine and dried over Na2SO4. The organic layer was concentrated under vacuum, and purified by column chromatography to obtain 2-hydroxy-N,N-dimethyl-4-nitrobenzamide (86 mg. 71% yield). 1H NMR (300 MHz, CDCl3) δ 10.15 (s, 1H), 7.85 (d, J=2.3 Hz, 1H), 7.73 (dd, J=8.5, 2.3 Hz, 1H), 7.49 (d, J=8.5 Hz, 1H), 3.21 (s, 6H).

Step 2. To a solution of methyl 2-hydroxy-4-nitrobenzoate (249 mg, 1.30 mmol) in DMF (6.5 mL) was added potassium carbonate (216 mg, 1.56 mmol) under nitrogen. The mixture was stirred for 10 minutes. Benzyl bromide (0.165 mL, 1.37 mmol) was added, and the mixture was stirred at room temperature for 5 hours. The mixture was poured onto cold water, and extracted with EtOAc (2×). The extract was washed with water (2×), brine, dried (sodium sulfate) and concentrated to dryness. Hexane trituration gave methyl 2-(benzyloxy)-4-nitrobenzoate as cream solid (329 mg, 88% yield). 1H NMR (300 MHz, Chloroform-d) δ 7.89 (dd, J=8.2, 2.0 Hz, 1H), 7.83 (d, J=2.0 Hz, 1H), 7.43 (d, J=8.3 Hz, 1H), 7.41-7.28 (m, 5H), 5.21 (s, 2H), 3.11 (s, 3H), 2.83 (s, 3H). HRMS (ESI) m/z=323.1000 [M+Na]+, HRMS (ESI+) calculated for C16H16N2O4: 300.11101, found 300.11088.

Step 3. Preparation by a similar procedure to Example 14, step 1, starting from methyl 2-(benzyloxy)-4-nitrobenzoate to obtain 4-amino-2-(benzyloxy)-N,N-dimethylbenzamide 1H NMR (300 MHz, Chloroform-d) δ 7.41-7.28 (m, 5H), 7.09 (d, J=8.1 Hz, 1H), 6.30 (dd, J=8.0, 2.0 Hz, 1H), 6.24 (d, J=2.0 Hz, 1H), 5.05 (s, 2H), 3.78 (s, 2H), 3.07 (s, 3H), 2.87 (s, 3H). HRMS (ESI) m/z=293.1260 [M+Na]+, HRMS (ESI+) calculated for C16H18N2O2: 270.13683, found 270.13692.

Step 4. Preparation by a similar procedure to Example 14, step 2, starting from 4-amino-2-(benzyloxy)-N,N-dimethylbenzamide to obtain 2-(benzyloxy)-N,N-dimethyl-4-(2,2,2-trifluoroacetamido)benzamide 1H NMR (300 MHz, Chloroform-d) δ 10.40 (s, 1H), 7.45-7.22 (m, 6H), 7.12-6.97 (m, 2H), 4.98-4.64 (m, 2H), 3.16 (s, 3H), 2.88 (s, 3H). HRMS (ESI) m/z=389.1082 [M+Na]+, HRMS (ESI+) calculated for C18H17F3N2O3: 366.11913, found 366.11929.

Step 5. Preparation by a similar procedure to Example 6, step 1, starting from 2-(benzyloxy)-N,N-dimethyl-4-(2,2,2-trifluoroacetamido)benzamide to obtain 2-(benzyloxy)-4-(N-((5-cyclohexylpyrazin-2-yl)methyl)-2,2,2-trifluoroacetamido)-N,N-dimethylbenzamide 1H NMR (300 MHz, Chloroform-d) δ 8.44 (d, J=1.5 Hz, 1H), 8.35 (d, J=1.5 Hz, 1H), 7.39-7.27 (m, 6H), 6.97-6.87 (m, 2H), 5.02 (s, 2H), 4.95 (s, 2H), 3.08 (s, 3H), 2.81 (s, 3H), 2.78-2.65 (m, 1H), 1.97-1.69 (m, 5H), 1.62-1.21 (m, 5H). 19F NMR (282 MHz, Chloroform-d) δ−67.03. HRMS (ESI) m/z=563.2224 [M+Na]+, HRMS (ESI+) calculated for C29H31F3N4O3: 540.23483, found 540.23295.

Step 6. Preparation by a similar procedure to Example 5, step 3, starting from 2-(benzyloxy)-4-(N-((5-cyclohexylpyrazin-2-yl)methyl)-2,2,2-trifluoroacetamido)-N,N-dimethylbenzamide to obtain 2-(benzyloxy)-4-(((5-cyclohexylpyrazin-2-yl)methyl)amino)-N,N-dimethylbenzamide 1H NMR (300 MHz, Chloroform-d) δ 8.48 (d, J=1.5 Hz, 1H), 8.39 (d, J=1.5 Hz, 1H), 7.38-7.23 (m, 5H), 7.11 (d, J=8.2 Hz, 1H), 6.28 (dd, J=8.2, 2.1 Hz, 1H), 6.24 (d, J=2.1 Hz, 1H), 5.02 (s, 2H), 4.91 (s, 1H), 4.41 (s, 2H), 3.05 (s, 3H), 2.86 (s, 3H), 2.80-2.65 (m, 1H), 1.97-1.69 (m, 5H), 1.64-1.17 (m, 5H). HRMS (ESI) m/z=467.2419[M+Na]+, HRMS (ESI+) calculated for C27H32N4O2: 444.25253, found 444.25256.

Step 7. Preparation by a similar procedure to Example 2, step 5, starting from 2-(benzyloxy)-4-(((5-cyclohexylpyrazin-2-yl)methyl)amino)-N,N-dimethylbenzamide to obtain tert-butyl (R)-2-((3-(benzyloxy)-4-(dimethylcarbamoyl)phenyl)((5-cyclohexylpyrazin-2-yl)methyl)carbamoyl)azetidine-1-carboxylate 1H NMR (300 MHz, Chloroform-d) δ 8.53 (s, 1H), 8.27 (d, J=1.5 Hz, 1H), 7.37-7.14 (m, 6H), 6.94-6.70 (m, 2H), 5.31-4.64 (m, 5H), 4.04-3.86 (m, 1H), 3.76-3.60 (m, 1H), 3.06 (s, 3H), 2.81 (s, 3H), 2.75-2.58 (m, 1H), 2.11-1.61 (m, 7H), 1.61-1.12 (m, 14H). HRMS (ESI) m/z=650.8310 [M+Na]+, HRMS (ESI+) calculated for C36H45N5O5: 627.34207, found 627.3418.

Step 8 and 9. Preparation by a similar procedure to Example 1, steps 6 and 7, starting from tert-butyl (R)-2-((3-(benzyloxy)-4-(dimethylcarbamoyl)phenyl)((5-cyclohexylpyrazin-2-yl)methyl)carbamoyl)azetidine-1-carboxylate to obtain (R)—N-(3-(benzyloxy)-4-(dimethylcarbamoyl)phenyl)-1-((3-cyano-4,5-difluorophenyl)sulfonyl)-N-((5-cyclohexylpyrazin-2-yl)methyl)azetidine-2-carboxamide. 1H NMR (300 MHz, Chloroform-d) δ 8.42 (d, J=1.5 Hz, 1H), 8.34 (d, J=1.5 Hz, 1H), 8.21-8.10 (m, 1H), 8.08-8.01 (m, 1H), 7.39-7.21 (m, 6H), 6.89-6.77 (m, 2H), 5.17-4.75 (m, 5H), 3.99-3.85 (m, 1H), 3.58-3.44 (m, 1H), 3.09 (s, 3H), 2.85 (s, 3H), 2.78-2.63 (m, 1H), 2.28-2.17 (m, 1H), 2.14-1.97 (m, 1H), 1.96-1.67 (m, 5H), 1.67-1.15 (m, 5H). 19F NMR (282 MHz, Chloroform-d) δ−122.64-−123.93 (m), −129.51-−130.79 (m). HRMS (ESI) m/z=751.252 [M+Na]+, HRMS (ESI+) calculated for C38H38F2N6O5S: 728.25925, found 728.26265.

Step 10. To a round-bottom flask containing a stir bar was added (R)—N-(3-(benzyloxy)-4-(dimethylcarbamoyl)phenyl)-1-((3-cyano-4,5-difluorophenyl) sulfonyl)-N-((5-cyclohexylpyrazin-2-yl)methyl)azetidine-2-carboxamide (0.213 mmol) in 1:4 ethyl acetate/methanol (8.8 mL). The mixture was exchanged with nitrogen gas three times before Pd/C (0.031 g) was added. The mixture was then exchanged with nitrogen gas three times before being exchanged with hydrogen gas three times and was then was allowed to stir at 25 degrees C. After 24 hours the reaction was complete, the mixture was flushed through a celite plug with ethyl acetate and concentrated in vacuo. Purification via pre-TLC yielded (R)-1-((3-cyano-4,5-difluorophenyl)sulfonyl)-N-((5-cyclohexylpyrazin-2-yl)methyl)-N-(4-(di methylcarbamoyl)-3-hydroxyphenyl)azetidine-2-carboxamide as a white solid in 44% yield. 1H NMR (300 MHz, Chloroform-d) δ 10.36 (s, 1H), 8.46 (d, J=1.5 Hz, 1H), 8.37 (d, J=1.5 Hz, 1H), 8.21-8.11 (m, 1H), 8.09-8.00 (m, 1H), 7.37 (d, J=8.3 Hz, 1H), 6.84 (d, J=2.1 Hz, 1H), 6.77 (dd, J=8.3, 2.2 Hz, 1H), 5.17-4.97 (m, 2H), 4.93-4.74 (m, 1H), 4.08-3.94 (m, 1H), 3.74-3.57 (m, 1H), 3.17 (s, 6H), 2.78-2.65 (m, 1H), 2.44-2.25 (m, 1H), 2.06-1.66 (m, 6H), 1.62-1.17 (m, 5H). 19F NMR (282 MHz, Chloroform-d) δ−122.85 (ddd, J=19.9, 7.1,

[symbol]

[symbol]

[symbol]

661.2015 [M+Na]+, HRMS (ESI+) calculated for C31H32F2N6O5S: 638.2123, found 638.21245.

### Example 17

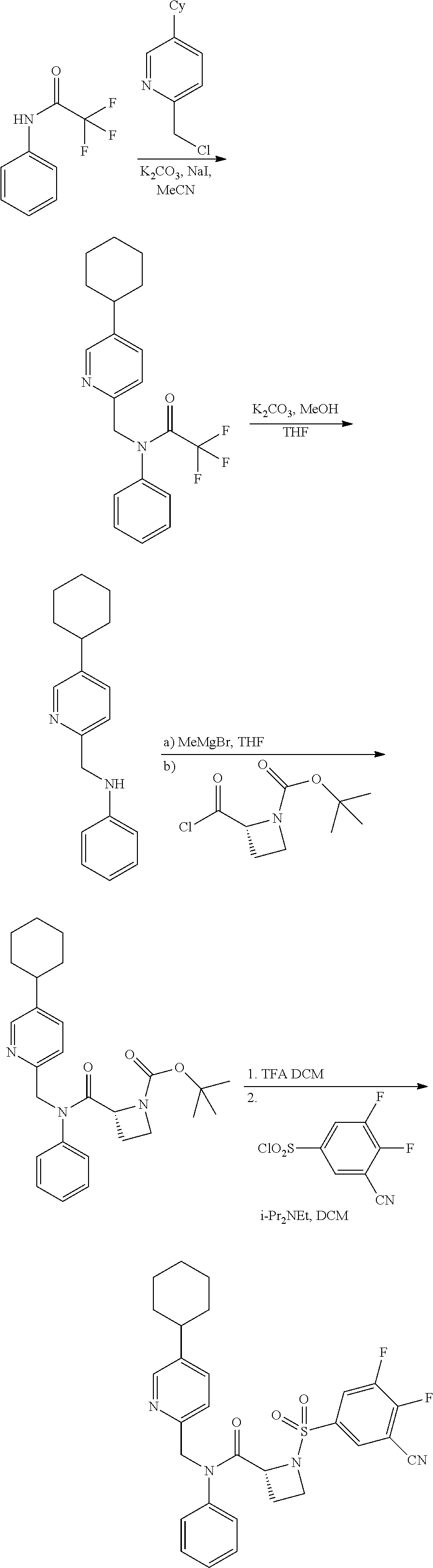

### (R)-1-((3-cyano-4,5-difluorophenyl)sulfonyl)-N-((5-cyclohexylpyridin-2-yl)methyl)-N-phenylazetidine-2-carboxamide

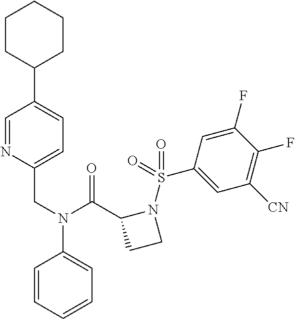 [SMILES: N#Cc1cc(S(=O)(=O)N2CC[C@@H]2C(=O)N(Cc2ccc(C3CCCCC3)cn2)c2ccccc2)cc(F)c1F]

Step 1. Preparation by a similar procedure to Example 5, step 2, starting from 2,2,2-trifluoro-N-phenylacetamide to obtain N-((5-cyclohexylpyridin-2-yl)methyl)-2,2,2-trifluoro-N-phenylacetamide. 1H NMR (300 MHz, Chloroform-d) δ 8.36 (d, J=2.3 Hz, 1H), 7.48 (dd, J=8.1, 2.3 Hz, 1H), 7.38-7.28 (m, 3H), 7.28-7.12 (m, 3H), 5.00 (s, 2H), 2.62-2.38 (m, 1H), 1.96-1.64 (m, 5H), 1.52-1.13 (m, 5H). 19F NMR (282 MHz, Chloroform-d) δ−66.95. HRMS (ESI) m/z=364.1718 [M+H]+, HRMS (ESI+) calculated for C20H21F3N2O: 362.1606, found 362.16056.

Step 2. Preparation by a similar procedure to Example 5, step 3, starting from N-((5-cyclohexylpyridin-2-yl)methyl)-2,2,2-trifluoro-N-phenylacetamide to obtain N-((5-cyclohexylpyridin-2-yl)methyl)aniline 1H NMR (300 MHz, Chloroform-d) δ 8.46 (d, J=2.3 Hz, 1H), 7.47 (dd, J=8.0, 2.3 Hz, 1H), 7.32-7.11 (m, 3H), 6.79-6.62 (m, 3H), 4.79 (s, 1H), 4.43 (s, 2H), 2.62-2.45 (m, 1H), 1.96-1.74 (m, 5H), 1.54-1.30 (m, 5H). HRMS (ESI) m/z=267.1854 [M+H]+, HRMS (ESI+) calculated for C18H22N2: 266.1783, found 266.1783.

Step 3. Preparation by a similar procedure to Example 2, step 5, starting from N-((5-cyclohexylpyridin-2-yl)methyl)aniline to obtain tert-butyl (R)-2-(((5-cyclohexylpyridin-2-yl)methyl)(phenyl)carbamoyl)azetidine-1-carboxylate, which was taken as such for next step.

Step 4. Preparation by a similar procedure to Example 1, step 6, starting from tert-butyl (R)-2-(((5-cyclohexylpyridin-2-yl)methyl)(phenyl)carbamoyl)azetidine-1-carboxylate to obtain (R)—N-((5-cyclohexylpyridin-2-yl)methyl)-N-phenylazetidine-2-carboxamide 1H NMR (300 MHz, Chloroform-d) δ 8.29 (d, J=2.2 Hz, 1H), 7.43 (dd, J=8.0, 2.3 Hz, 1H), 7.33-7.19 (m, 4H), 7.10-6.96 (m, 2H), 5.12-4.83 (m, 2H), 4.31-4.14 (m, 1H), 3.63-3.43 (m, 1H), 3.37-3.18 (m, 1H), 3.05 (s, 1H), 2.53-2.39 (m, 1H), 2.39-2.26 (m, 1H), 2.24-2.07 (m, 1H), 1.87-1.63 (m, 5H), 1.47-1.24 (m, 5H). HRMS (ESI) m/z=350.2228 [M+H]+, HRMS (ESI+) calculated for C22H27N3O: 349.21541, found 349.21558.

Step 5. Preparation by a similar procedure to Example 1, step 7, starting from (R)—N-((5-cyclohexylpyridin-2-yl)methyl)-N-phenylazetidine-2-carboxamide to obtain (R)-1-((3-cyano-4,5-difluorophenyl)sulfonyl)-N-((5-cyclohexylpyridin-2-yl)methyl)-N-phenylazetidine-2-carboxamide. 1H NMR (300 MHz, Chloroform-d) δ 8.33 (d, J=2.3 Hz, 1H), 8.22-8.11 (m, 1H), 8.07-7.96 (m, 1H), 7.48 (dd, J=8.0, 2.3 Hz, 1H), 7.41-7.32 (m, 3H), 7.25 (d, J=8.5 Hz, 1H), 7.19-7.11 (m, 2H), 5.03-4.87 (m, 3H), 4.02-3.84 (m, 1H), 3.72-3.57 (m, 1H), 2.56-2.42 (m, 1H), 2.41-2.24 (m, 1H), 1.92-1.66 (m, 6H), 1.49-1.25 (m, 5H). 19F NMR (282 MHz, Chloroform-d) δ−123.05 (ddd, J=20.0, 7.0, 5.0 Hz), −129.95 (ddd, J=20.0, 9.0, 1.8 Hz). HPLC Purity: 100%. HRMS (ESI) m/z=573.1740 [M+Na]+, HRMS (ESI+) calculated for C29H28F2N4O3S: 550.18502, found 550.1849.

### Example 18

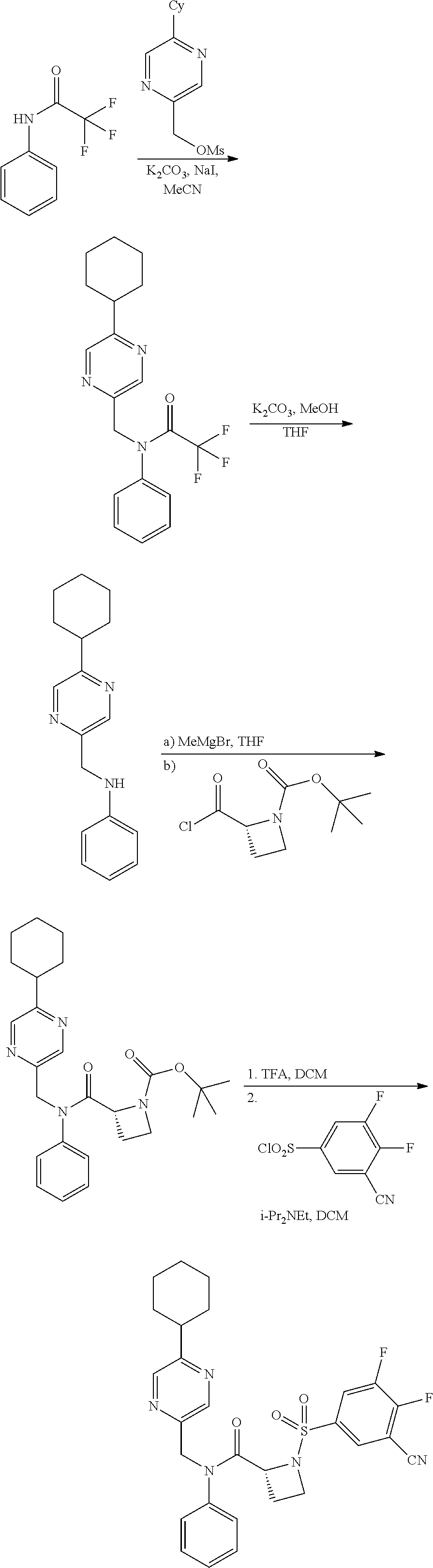

### (R)-1-((3-cyano-4,5-difluorophenyl)sulfonyl)-N-((5-cyclohexylpyrazin-2-yl)methyl)-N-phenylazetidine-2-carboxamide

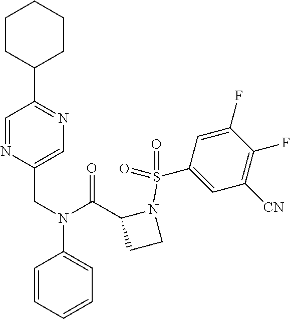 [SMILES: N#Cc1cc(S(=O)(=O)N2CC[C@@H]2C(=O)N(Cc2cnc(C3CCCCC3)cn2)c2ccccc2)cc(F)c1F]

Step 1. Preparation by a similar procedure to Example 6, step 1, starting from 2,2,2-trifluoro-N-phenylacetamide to obtain N-((5-cyclohexylpyrazin-2-yl)methyl)-2,2,2-trifluoro-N-phenylacetamide. 1H NMR (300 MHz, Chloroform-d) δ 8.44 (d, J=1.5 Hz, 1H), 8.37 (d, J=1.5 Hz, 1H), 7.39-7.28 (m, 3H), 7.27-7.18 (m, 2H), 4.99 (s, 2H), 2.77-2.63 (m, 1H), 1.95-1.66 (m, 5H), 1.61-1.17 (m, 5H). 19F NMR (282 MHz, Chloroform-d) δ−67.09. HRMS (ESI) m/z=364.1466 [M+Na]+, HRMS (ESI+) calculated for C19H20F3N3O: 363.15585, found 363.15622.

Step 2. Preparation by a similar procedure to Example 5, step 3, starting from N-((5-cyclohexylpyrazin-2-yl)methyl)-2,2,2-trifluoro-N-phenylacetamide to obtain N-((5-cyclohexylpyrazin-2-yl)methyl)aniline. 1H NMR (300 MHz, Chloroform-d) δ 8.53 (d, J=1.4 Hz, 1H), 8.41 (d, J=1.5 Hz, 1H), 7.24-7.12 (m, 2H), 6.79-6.57 (m, 3H), 4.71 (s, 1H), 4.44 (s, 2H), 2.84-2.63 (m, 1H), 2.01-1.70 (m, 5H), 1.66-1.18 (m, 5H). HRMS (ESI) m/z=268.1810 [M+H]+, HRMS (ESI+) calculated for C17H21N3: 267.17355, found 267.17373.

Step 3. Preparation by a similar procedure to Example 2, step 5, starting from N-((5-cyclohexylpyrazin-2-yl)methyl)aniline to obtain tert-butyl (R)-2-(((5-cyclohexylpyrazin-2-yl)methyl)(phenyl)carbamoyl)azetidine-1-carboxylate. 1H NMR (300 MHz, Chloroform-d) δ 8.47 (d, J=1.4 Hz, 1H), 8.32 (d, J=1.5 Hz, 1H), 7.35-7.23 (m, 3H), 7.13-7.04 (m, 2H), 5.16-4.79 (m, 2H), 4.29-4.14 (m, 1H), 3.61-3.44 (m, 1H), 3.37-3.24 (m, 1H), 3.21 (s, 1H), 2.78-2.58 (m, 1H), 2.44-2.26 (m, 1H), 2.26-2.14 (m, 1H), 1.93-1.64 (m, 5H), 1.60-1.13 (m, 5H). HRMS (ESI) m/z=373.1998 [M+Na]+, HRMS (ESI+) calculated for C21H26N4O: 350.21066, found 350.21009.

Step 4. Preparation by a similar procedure to Example 1, steps 6 and 7, starting from tert-butyl (R)-2-(((5-cyclohexylpyrazin-2-yl)methyl)(phenyl)carbamoyl)azetidine-1-carboxylate to obtain (R)-1-((3-cyano-4,5-difluorophenyl)sulfonyl)-N-((5-cyclohexylpyrazin-2-yl)methyl)-N-phenylazetidine-2-carboxamide. 1H NMR (300 MHz, Chloroform-d) δ 8.46 (d, J=1.5 Hz, 1H), 8.37 (d, J=1.5 Hz, 1H), 8.22-8.13 (m, 1H), 8.07-8.00 (m, 1H), 7.45-7.36 (m, 3H), 7.23-7.16 (m, 2H), 5.20-4.82 (m, 3H), 4.02-3.88 (m, 1H), 3.71-3.55 (m, 1H), 2.80-2.64 (m, 1H), 2.40-2.24 (m, 1H), 2.01-1.70 (m, 6H), 1.58-1.20 (m, 5H). 19F NMR (282 MHz, Chloroform-d) δ−122.82 (ddd, J=20.0, 7.0, 4.8 Hz), −129.83 (ddd, J=20.1, 8.8, 1.8 Hz). HPLC Purity=100%. HRMS (ESI) m/z=574.1698 [M+Na]+, HRMS (ESI+) calculated for C28H27F2N5O3S: 551.18027, found 551.18083.

### Example 19

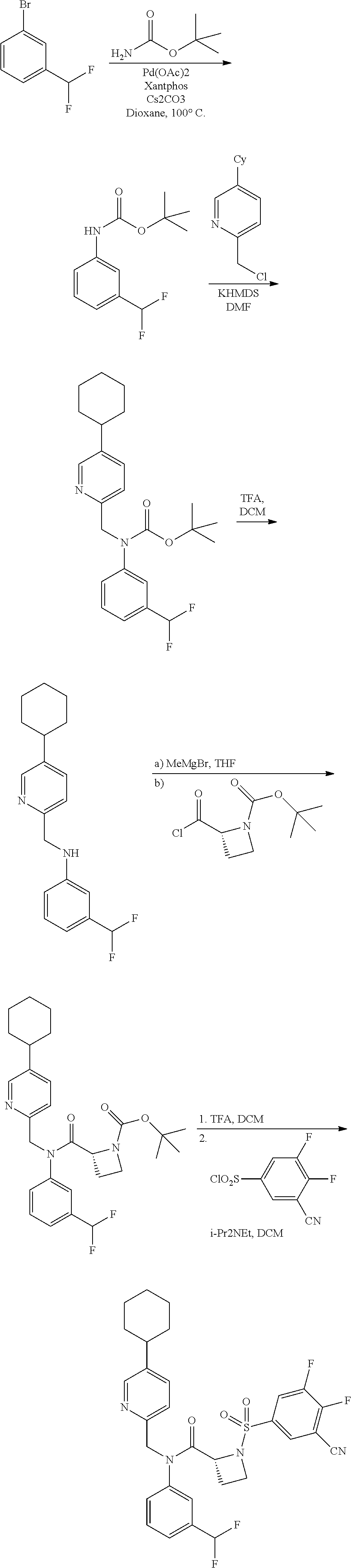

### (R)-1-((3-cyano-4,5-difluorophenyl)sulfonyl)-N-((5-cyclohexylpyridin-2-yl)methyl)-N-(3-(difluoromethyl)phenyl)azetidine-2-carboxamide

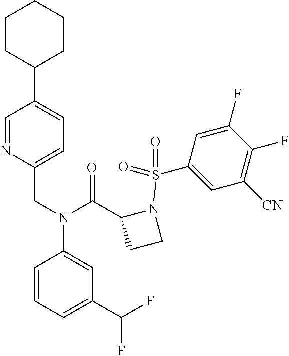 [SMILES: N#Cc1cc(S(=O)(=O)N2CC[C@@H]2C(=O)N(Cc2ccc(C3CCCCC3)cn2)c2cccc(C(F)F)c2)cc(F)c1F]

Step 1. Preparation by a similar procedure to Example 4, step 2, starting from 1-bromo-3-(difluoromethyl)benzene to obtain tert-butyl (3-(difluoromethyl)phenyl)carbamate. 1H NMR (300 MHz, Chloroform-d) δ 7.61 (s, 1H), 7.44-7.32 (m, 2H), 7.18 (d, J=6.8 Hz, 1H), 6.82-6.38 (m, 2H), 1.52 (s, 9H). 19F NMR (282 MHz, Chloroform-d) δ−110.81 (d, J=56.6 Hz).

Step 2. Preparation by a similar procedure to Example 2, step 3, starting from tert-butyl (3-(difluoromethyl)phenyl)carbamate to obtain tert-butyl ((5-cyclohexylpyridin-2-yl)methyl)(3-(difluoromethyl)phenyl)carbamate. 1H NMR (300 MHz, Chloroform-d) δ 8.37 (d, J=2.3 Hz, 1H), 7.52-7.43 (m, 2H), 7.42-7.19 (m, 4H), 6.56 (t, J=56.5 Hz, 1H), 4.92 (s, 2H), 2.57-2.43 (m, 1H), 1.91-1.67 (m, 5H), 1.51-1.14 (m, 14H). 19F NMR (282 MHz, Chloroform-d) δ−110.92 (d, J=56.3 Hz). HRMS (ESI) m/z=417.2354 [M+H]+, HRMS (ESI+) calculated for C24H30F2N2O2: 416.22753, found 416.22802.

Step 3. Preparation by a similar procedure to Example 4, step 4, starting from tert-butyl ((5-cyclohexylpyridin-2-yl)methyl)(3-(difluoromethyl)phenyl)carbamate to obtain N-((5-cyclohexylpyridin-2-yl)methyl)-3-(difluoromethyl)aniline 1H NMR (300 MHz, Chloroform-d) δ 8.44 (d, J=2.2 Hz, 1H), 7.47 (dd, J=8.0, 2.3 Hz, 1H), 7.29-7.15 (m, 2H), 6.89-6.30 (m, 4H), 5.08 (s, 1H), 4.41 (s, 2H), 2.59-2.45 (m, 1H), 1.95-1.69 (m, 5H), 1.53-1.28 (m, 5H). 19F NMR (282 MHz, Chloroform-d) δ−110.45 (d, J=56.7 Hz). HRMS (ESI) m/z=317.1825 [M+H]+, HRMS (ESI+) calculated for C19H22F2N2: 316.17511, found 316.17533.

Step 4. Preparation by a similar procedure to Example 2, step 5, starting from N-((5-cyclohexylpyridin-2-yl)methyl)-3-(difluoromethyl)aniline to obtain tert-butyl (R)-2-(((5-cyclohexylpyridin-2-yl)methyl) (3-(difluoromethyl)phenyl)carbamoyl)azetidine-1-carboxylate, which was taken as such to next step.

Step 5. Preparation by a similar procedure to Example 1, step 6, starting from tert-butyl (R)-2-(((5-cyclohexylpyridin-2-yl)methyl)(3-(difluoromethyl)phenyl)carbamoyl)azetidine-1-carboxylate to obtain (R)—N-((5-cyclohexylpyridin-2-yl)methyl)-N-(3-(difluoromethyl)phenyl)azetidine-2-carboxamide. 1H NMR (300 MHz, Chloroform-d) δ 8.40-8.20 (m, 1H), 7.59-7.10 (m, 7H), 6.55 (td, J=56.3, 4.6 Hz, 1H), 5.17-4.77 (m, 2H), 3.25-2.85 (m, 3H), 2.58-2.29 (m, 1H), 2.25-2.01 (m, 1H), 1.90-1.60 (m, 6H), 1.51-1.08 (m, 5H). 19F NMR (282 MHz, Chloroform-d) δ−110.65-−111.68 (m). HRMS (ESI) m/z=422.2017 [M+Na]+, HRMS (ESI+) calculated for C23H27F2N3O: 399.21222, found 399.21255.

Step 6. Preparation by a similar procedure to Example 1, step 7, starting from (R)—N-((5-cyclohexylpyridin-2-yl)methyl)-N-(3-(difluoromethyl)phenyl)azetidine-2-carboxamide to obtain (R)-1-((3-cyano-4,5-difluorophenyl)sulfonyl)-N-((5-cyclohexylpyridin-2-yl)methyl)-N-(3-(difluoromethyl)phenyl)azetidine-2-carboxamide. 1H NMR (300 MHz, Chloroform-d) δ 8.34 (d, J=2.2 Hz, 1H), 8.23-8.10 (m, 1H), 8.08-7.99 (m, 1H), 7.58-7.18 (m, 7H), 6.60 (t, J=56.1 Hz, 1H), 5.10-4.79 (m, 3H), 4.06-3.86 (m, 1H), 3.75-3.52 (m, 1H), 2.60-2.44 (m, 1H), 2.43-2.25 (m, 1H), 1.97-1.60 (m, 7H), 1.53-1.13 (m, 7H). 19F NMR (282 MHz, Chloroform-d) δ−111.30 (d, J=42.4 Hz), −111.50 (d, J=42.4 Hz), −123.02 (ddd, J=19.9, 7.1, 5.0 Hz), −129.77-−130.21 (m). HPLC Purity=100%. HRMS (ESI) m/z=623.1710 [M+Na]+, HRMS (ESI+) calculated for C30H28F4N4O3S: 600.18182, found 600.18192.

### Example 20

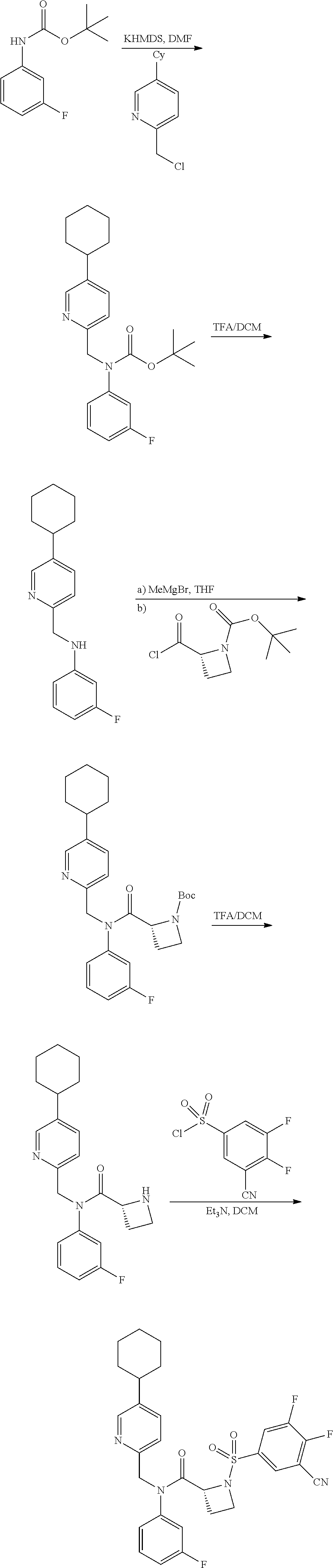

### (R)-1-((3-cyano-4,5-difluorophenyl)sulfonyl)-N-((5-cyclohexylpyridin-2-yl)methyl)-N-(3-fluorophenyl)azetidine-2-carboxamide

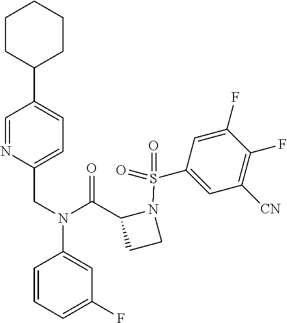 [SMILES: N#Cc1cc(S(=O)(=O)N2CC[C@@H]2C(=O)N(Cc2ccc(C3CCCCC3)cn2)c2cccc(F)c2)cc(F)c1F]

Step 1. Preparation by a similar procedure to Example 2, step 3, starting from tert-butyl (3-fluorophenyl)carbamate to obtain tert-butyl ((5-cyclohexylpyridin-2-yl)methyl)(3-fluorophenyl)carbamate. 1H NMR (300 MHz, Chloroform-d) δ 8.33 (d, J=2.2 Hz, 1H), 7.42 (dd, J=8.1, 2.3 Hz, 1H), 7.21-6.97 (m, 4H), 6.74 (tdd, J=8.2, 2.5, 1.0 Hz, 1H), 4.86 (s, 2H), 2.53-2.35 (m, 1H), 1.86-1.61 (m, 5H), 1.45-1.10 (m, 14H). 19F NMR (282 MHz, Chloroform-d) δ−112.20-−112.82 (m). HRMS (ESI) m/z=407.2109 [M+Na]+, HRMS (ESI+) calculated for C23H29FN2O2: 384.22131, found 384.22171.

Step 2. Preparation by a similar procedure to Example 4, step 4, starting from tert-butyl ((5-cyclohexylpyridin-2-yl)methyl)(3-fluorophenyl)carbamate to obtain N-((5-cyclohexylpyridin-2-yl)methyl)-3-fluoroaniline 1H NMR (300 MHz, Chloroform-d) δ 8.43 (d, J=2.3 Hz, 1H), 7.49 (dd, J=8.0, 2.3 Hz, 1H), 7.23 (d, J=8.0 Hz, 1H), 7.15-7.04 (m, 1H), 6.51-6.28 (m, 3H), 4.94 (s, 1H), 4.39 (s, 2H), 2.60-2.45 (m, 1H), 1.95-1.69 (m, 5H), 1.52-1.19 (m, 6H). 19F NMR (282 MHz, Chloroform-d) δ−112.97 (ddd, J=11.5, 8.8, 6.8 Hz). HRMS (ESI) m/z=285.1766 [M+H]+, HRMS (ESI+) calculated for C18H21FN2: 284.16888, found 284.16936.

Step 3. Preparation by a similar procedure to Example 2, step 5, starting from N-((5-cyclohexylpyridin-2-yl)methyl)-3-fluoroaniline to obtain tert-butyl (R)-2-(((5-cyclohexylpyridin-2-yl)methyl)(3-fluorophenyl)carbamoyl)azetidine-1-carboxylat e. 1H NMR (300 MHz, Chloroform-d) δ 8.30 (d, J=2.2 Hz, 1H), 7.47 (dd, J=8.0, 2.3 Hz, 1H), 7.43-7.25 (m, 2H), 7.09-6.83 (m, 3H), 5.11-4.81 (m, 2H), 4.68-4.45 (m, 1H), 4.14-3.96 (m, 1H), 3.82-3.64 (m, 1H), 2.57-2.40 (m, 1H), 2.23-2.03 (m, 2H), 1.92-1.64 (m, 5H), 1.52-1.10 (m, 14H). 19F NMR (282 MHz, Chloroform-d) δ−110.17-−110.60 (m).

Step 4 and 5. Preparation by a similar procedure to Example 1, steps 6 and 7, starting from tert-butyl (R)-2-(((5-cyclohexylpyridin-2-yl)methyl)(3-fluorophenyl)carbamoyl)azetidine-1-carboxylat e to obtain (R)-1-((3-cyano-4,5-difluorophenyl)sulfonyl)-N-((5-cyclohexylpyridin-2-yl)methyl)-N-(3-fluorophenyl)azetidine-2-carboxamide 1H NMR (300 MHz, Chloroform-d) δ 8.35 (d, J=2.3 Hz, 1H), 8.21-8.10 (m, 1H), 8.08-8.00 (m, 1H), 7.49 (dd, J=8.0, 2.3 Hz, 1H), 7.42-7.30 (m, 1H), 7.23 (d, J=8.0 Hz, 1H), 7.14-6.90 (m, 3H), 5.11-4.81 (m, 3H), 4.11-3.85 (m, 1H), 3.77-3.56 (m, 1H), 2.58-2.44 (m, 1H), 2.43-2.24 (m, 1H), 2.09-1.61 (m, 6H), 1.52-1.10 (m, 5H). 19F NMR (282 MHz, Chloroform-d) δ−108.76-−110.07 (m), −122.96 (ddd, J=19.9, 7.0, 4.9 Hz), −129.95 (ddd, J=19.6, 8.8, 1.8 Hz). [M+H]=569.1. HPLC purity=100%

### Example 21

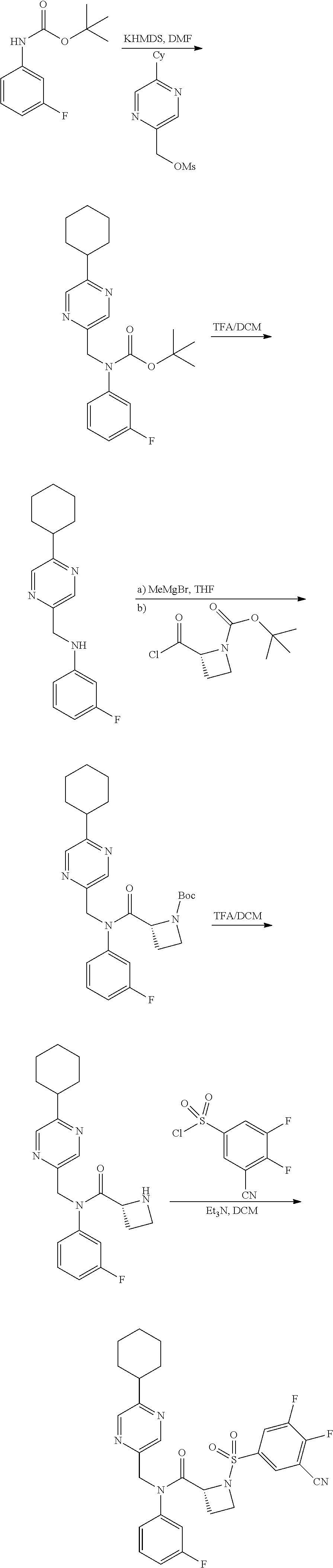

### (R)-1-((3-cyano-4,5-difluorophenyl)sulfonyl)-N-((5-cyclohexylpyrazin-2-yl)methyl)-N-(3-fluorophenyl)azetidine-2-carboxamide

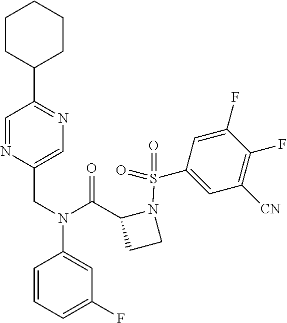 [SMILES: N#Cc1cc(S(=O)(=O)N2CC[C@@H]2C(=O)N(Cc2cnc(C3CCCCC3)cn2)c2cccc(F)c2)cc(F)c1F]

Step 1. Preparation by a similar procedure to Example 2, step 3, starting from tert-butyl (3-fluorophenyl)carbamate to obtain tert-butyl ((5-cyclohexylpyrazin-2-yl)methyl)(3-fluorophenyl)carbamate. 1H NMR (300 MHz, Chloroform-d) δ 8.47 (d, J=1.5 Hz, 1H), 8.34 (d, J=1.5 Hz, 1H), 7.24-6.98 (m, 3H), 6.86-6.73 (m, 1H), 4.88 (s, 2H), 2.77-2.59 (m, 1H), 1.97-1.08 (m, 19H). 19F NMR (282 MHz, Chloroform-d) δ−112.10-−112.39 (m).

Step 2. Preparation by a similar procedure to Example 4, step 4, starting from tert-butyl ((5-cyclohexylpyrazin-2-yl)methyl)(3-fluorophenyl)carbamate to obtain N-((5-cyclohexylpyrazin-2-yl)methyl)-3-fluoroaniline. 1H NMR (300 MHz, Chloroform-d) δ 8.49 (d, J=1.4 Hz, 1H), 8.39 (d, J=1.5 Hz, 1H), 7.13-6.99 (m, 1H), 6.48-6.28 (m, 3H), 4.89 (t, J=5.4 Hz, 1H), 4.39 (d, J=4.8 Hz, 2H), 2.81-2.62 (m, 1H), 2.02-1.14 (m, 10H). 19F NMR (282 MHz, Chloroform-d) δ−112.57 (ddd, J=11.3, 8.6, 6.7 Hz).

Step 3. Preparation by a similar procedure to Example 2, step 5, starting from N-((5-cyclohexylpyrazin-2-yl)methyl)-3-fluoroaniline to obtain tert-butyl (R)-2-(((5-cyclohexylpyrazin-2-yl)methyl)(3-fluorophenyl)carbamoyl)azetidine-1-carboxylat e. 1H NMR (300 MHz, Chloroform-d) δ 8.54 (s, 1H), 8.29 (s, 1H), 7.36-7.26 (m, 1H), 7.12-6.86 (m, 3H), 5.32-4.63 (m, 3H), 4.60-4.46 (m, 1H), 4.06-3.90 (m, 1H), 3.77-3.56 (m, 1H), 2.73-2.56 (m, 1H), 2.20-1.99 (m, 2H), 1.92-1.15 (m, 19H). 19F NMR (282 MHz, Chloroform-d) δ−109.68-−110.36 (m).

Step 4 and 5. Preparation by a similar procedure to Example 1, steps 6 and 7, starting from tert-butyl (R)-2-(((5-cyclohexylpyrazin-2-yl)methyl)(3-fluorophenyl)carbamoyl)azetidine-1-carboxylate to obtain (R)-1-((3-cyano-4,5-difluorophenyl)sulfonyl)-N-((5-cyclohexylpyrazin-2-yl)methyl)-N-(3-fluorophenyl)azetidine-2-carboxamide 1H NMR (300 MHz, Chloroform-d) δ 8.46 (s, 1H), 8.37 (s, 1H), 8.20-8.09 (m, 1H), 8.07-7.99 (m, 1H), 7.47-7.32 (m, 1H), 7.19-6.94 (m, 3H), 5.22-4.77 (m, 3H), 4.12-3.87 (m, 1H), 3.76-3.54 (m, 1H), 2.83-2.60 (m, 1H), 2.44-2.22 (m, 1H), 2.03-1.66 (m, 6H), 1.66-1.14 (m, 5H). 19F NMR (282 MHz, Chloroform-d) δ−108.78-−109.32 (m), −122.84 (ddd, J=20.0, 7.1, 4.9 Hz), −129.87 (ddd, J=20.0, 9.0, 1.8 Hz).

### Example 22

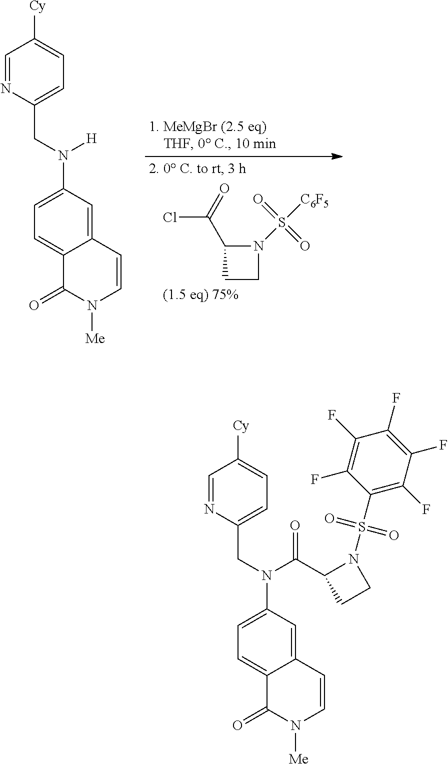

### (R)—N-((5-cyclohexylpyridin-2-yl)methyl)-N-(2-methyl-1-oxo-1,2-dihydroisoquinolin-6-yl)-1-((perfluorophenyl)sulfonyl)azetidine-2-carboxamide

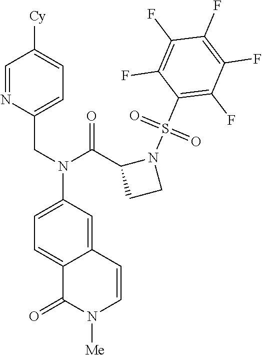

Step 1. To a solution of 6-(((5-cyclohexylpyridin-2-yl)methyl)amino)-2-methylisoquinolin-1(2H)-one (100 mg, 0.29 mmol, 1.00 equiv) in 3 mL THF was added MeMgBr (0.5 mL, 0.72 mmol, 2.5 equiv) at 0 degrees C. under Argon. After 10 min, (R)-1-((perfluorophenyl)sulfonyl)azetidine-2-carbonyl chloride (151 mg, 0.43 mmol, 1.5 equiv) was added to the reaction at 0 degrees C. under Argon. The reaction was allowed to warm up to room temperature and stirred for 3 h. Then the reaction was quenched with saturated NH4Cl aq, and extracted with EtOAc (3×). The combined organic extracts were washed with saturated brine, dried over anhydrous sodium sulfate, and concentrated under reduced pressure. The residue was purified by flash chromatography (eluent: DCM/MeOH 80/1) to provide (R)—N-((5-cyclohexylpyridin-2-yl)methyl)-N-(2-methyl-1-oxo-1,2-dihydroisoquinolin-6-yl)-1-((perfluorophenyl)sulfonyl)azetidine-2-carboxamide as light yellow solid (142 mg, 75%). 1H NMR (300 MHz, CDCl3) δ 8.37 (d, J=8.5 Hz, 1H), 8.30 (s, 1H), 7.50 (dd, J=7.9, 1.5 Hz, 1H), 7.34 (s, 1H), 7.24-7.07 (m, 3H), 6.40 (d, J=7.3 Hz, 1H), 5.13-4.83 (m, 3H), 4.13-3.97 (m, 2H), 3.58 (s, 3H), 2.58-2.41 (m, 1H), 2.38-2.20 (m, 1H), 1.99-1.54 (m, 7H), 1.47-1.29 (m, 4H). LRMS (ESI) m/z 661.3 [M+H]+; HRMS (ESI) m/z 661.1886 [M+H]+, 683.1670 [M+Na]+; Purity 100%.

### Example 23

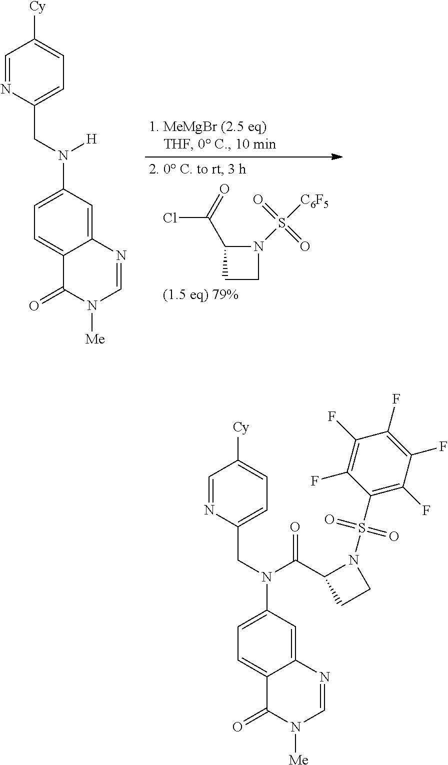

### (R)—N-((5-cyclohexylpyridin-2-yl)methyl)-N-(3-methyl-4-oxo-3,4-dihydroquinazolin-7-yl)-1-((perfluorophenyl)sulfonyl)azetidine-2-carboxamide

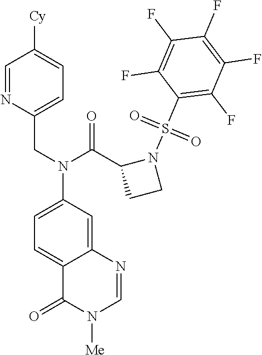

Step 1. To a solution of 7-(((5-cyclohexylpyridin-2-yl)methyl)amino)-3-methylquinazolin-4(3H)-one (100 mg, 0.29 mmol, 1.00 equiv) in 3 mL THF was added MeMgBr (0.5 mL, 0.72 mmol, 2.5 equiv) at 0 degrees C. under Argon. After 10 min, (R)-1-((perfluorophenyl)sulfonyl)azetidine-2-carbonyl chloride (151 mg, 0.43 mmol, 1.5 equiv) was added to the reaction at 0 degrees C. under Argon. The reaction was allowed to warm up to room temperature and stirred for 3 h. Then the reaction was quenched with saturated NH4Cl aq, and extracted with EtOAc (3×). The combined organic extracts were washed with saturated brine, dried over anhydrous sodium sulfate, and concentrated under reduced pressure. The residue was purified by flash chromatography (eluent: DCM/MeOH 80/1) to provide (R)—N-((5-cyclohexylpyridin-2-yl)methyl)-N-(3-methyl-4-oxo-3,4-dihydroquinazolin-7-yl)-1-((perfluorophenyl)sulfonyl)azetidine-2-carboxamide as light yellow solid (150 mg, 79%). 1H NMR (500 MHz, CDCl3) δ 8.38-8.25 (m, 2H), 8.06 (s, 1H), 7.60-7.53 (m, 1H), 7.51 (s, 1H), 7.32 (d, J=7.9 Hz, 1H), 7.26 (s, 1H), 5.10-4.92 (m, 3H), 4.16-4.00 (m, 2H), 3.59 (s, 3H), 2.57-2.44 (m, 1H), 2.44-2.28 (m, 1H), 2.07-1.92 (m, 1H), 1.91-1.71 (m, 6H), 1.45-1.35 (m, 4H). LRMS (ESI) m/z 662.3 [M+H]+; HRMS (ESI) m/z 662.1847 [M+H]+, 684.1734 [M+Na]+; Purity 100%.

### Example 24

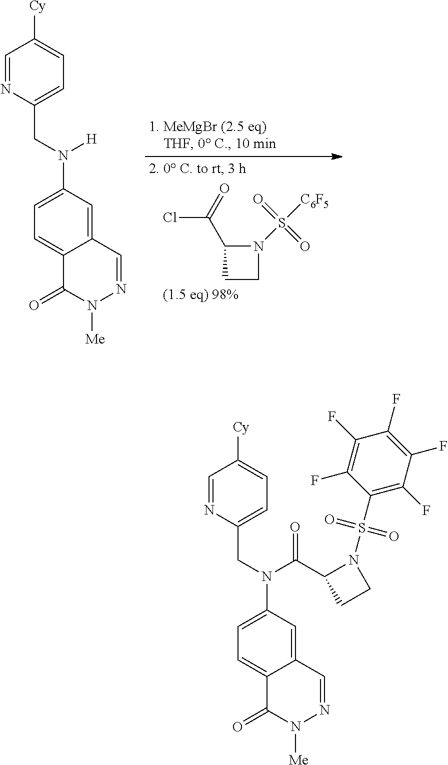

### (R)—N-((5-cyclohexylpyridin-2-yl)methyl)-N-(2-methyl-1-oxo-1,2-dihydrophthalazin-6-yl)-1-((perfluorophenyl)sulfonyl)azetidine-2-carboxamide

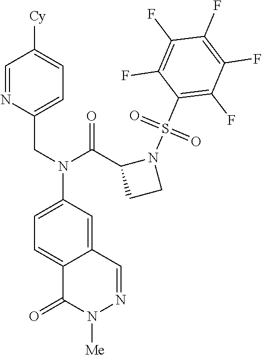

Step 1. To a solution of 6-(((5-cyclohexylpyridin-2-yl)methyl)amino)-2-methylphthalazin-1(2H)-one (100 mg, 0.29 mmol, 1.00 equiv) in 3 mL THF was added MeMgBr (0.5 mL, 0.72 mmol, 2.5 equiv) at 0 degrees C. under Argon. After 10 min, (R)-1-((perfluorophenyl)sulfonyl)azetidine-2-carbonyl chloride (151 mg, 0.43 mmol, 1.5 equiv) was added to the reaction at 0 degrees C. under Argon. The reaction was allowed to warm up to room temperature and stirred for 3 h. Then the reaction was quenched with saturated NH4Cl aq, and extracted with EtOAc (3×). The combined organic extracts were washed with saturated brine, dried over anhydrous sodium sulfate, and concentrated under reduced pressure. The residue was purified by flash chromatography (eluent: DCM/MeOH 100/1) to provide (R)—N-((5-cyclohexylpyridin-2-yl)methyl)-N-(2-methyl-1-oxo-1,2-dihydrophthalazin-6-yl)-1-((perfluorophenyl)sulfonyl)azetidine-2-carboxamide as light yellow solid (186 mg, 98%). 1H NMR (500 MHz, CDCl3) δ 8.41 (d, J=8.3 Hz, 1H), 8.32 (s, 1H), 8.08 (s, 1H), 7.61 (s, 1H), 7.57-7.49 (m, 2H), 7.23 (d, J=7.1 Hz, 1H), 5.04-4.90 (m, 3H), 4.10-4.00 (m, 2H), 3.83 (s, 3H), 2.55-2.46 (m, 1H), 2.37-2.27 (m, 1H), 2.01-1.89 (m, 1H), 1.89-1.64 (m, 6H), 1.42-1.34 (m, 4H). LRMS (ESI) m/z 662.3 [M+H]+; HRMS (ESI) m/z 662.1854 [M+H]+, 684.1675 [M+Na]+; Purity 100%.

### Example 25

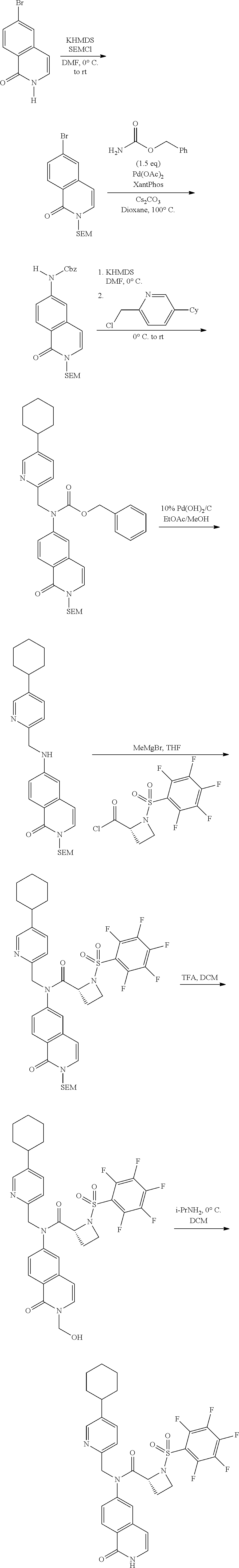

### (R)—N-((5-cyclohexylpyridin-2-yl)methyl)-N-(1-oxo-1,2-dihydroisoquinolin-6-yl)-1-((perfluorophenyl)sulfonyl)azetidine-2-carboxamide

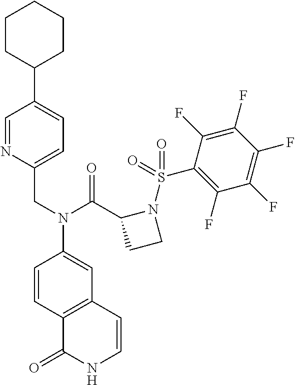 [SMILES: O=C([C@H]1CCN1S(=O)(=O)c1c(F)c(F)c(F)c(F)c1F)N(Cc1ccc(C2CCCCC2)cn1)c1ccc2c(=O)[nH]ccc2c1]

Step 1. Preparation by a similar procedure to Example 2, step 1, starting from 6-bromoisoquinolin-1(2H)-one to obtain 6-bromo-2-((2-(trimethylsilyl)ethoxy)methyl)isoquinolin-1(2H)-one (70% yield) as white solid. 1H NMR (300 MHz, CDCl3) δ 8.27 (d, J=8.5 Hz, 1H), 7.69-7.67 (m, 1H), 7.60-7.55 (m, 1H), 7.23 (d, J=7.5 Hz, 1H), 6.44 (d, J=7.4 Hz, 1H), 5.41 (s, 2H), 3.66-3.59 (m, 2H), 0.98-0.91 (m, 2H), −0.00-−0.03 (m, 9H).

Step 2. Preparation by a similar procedure to Example 2, step 2, starting from 6-bromo-2-((2-(trimethylsilyl)ethoxy)methyl)isoquinolin-1(2H)-one to obtain benzyl (1-oxo-2-((2-(trimethylsilyl)ethoxy)methyl)-1,2-dihydroisoquinolin-6-yl)carbamate (87% yield) as a white solid. 1H NMR (300 MHz, CDCl3) δ 8.36 (d, J=8.7 Hz, 1H), 7.83 (s, 1H), 7.48-7.37 (m, 5H), 7.31-7.28 (m, 1H), 7.19 (d, J=7.5 Hz, 1H), 7.11-6.98 (m, 1H), 6.49 (d, J=7.4 Hz, 1H), 5.42 (s, 2H), 5.25 (s, 2H), 3.77-3.58 (m, 2H), 1.04-0.89 (m, 2H), −0.01 (s, 9H).

Step 3. Preparation by a similar procedure to Example 2, step 3, starting from benzyl (1-oxo-2-((2-(trimethylsilyl)ethoxy)methyl)-1,2-dihydroisoquinolin-6-yl)carbamate to obtain benzyl ((5-cyclohexylpyridin-2-yl)methyl)(1-oxo-2-((2-(trimethylsilyl)ethoxy)methyl)-1,2-dihydroisoquinolin-6-yl)carbamate (87% yield) as light yellow oil. 1H NMR (300 MHz, Chloroform-d) δ 8.41 (d, J=2.3 Hz, 1H), 8.35 (d, J=8.8 Hz, 1H), 7.51-7.40 (m, 3H), 7.37-7.13 (m, 7H), 6.44 (d, J=7.5 Hz, 1H), 5.42 (s, 2H), 5.23 (s, 2H), 5.09 (s, 2H), 3.69-3.57 (m, 2H), 2.61-2.46 (m, 1H), 1.92-1.73 (m, 5H), 1.53-1.31 (m, 5H), 1.02-0.89 (m, 2H), −0.01 (s, 9H).

Step 4. Preparation by a similar procedure to Example 2, step 4, starting from benzyl ((5-cyclohexylpyridin-2-yl)methyl)(1-oxo-2-((2-(trimethylsilyl)ethoxy)methyl)-1,2-dihydroisoquinolin-6-yl)carbamate (1.55 g) to obtain 6-(((5-cyclohexylpyridin-2-yl)methyl)amino)-2-((2-(trimethylsilyl)ethoxy)methyl)isoquinolin-1(2H)-one (1.08 g, 90%). 1H NMR (300 MHz, Chloroform-d) δ 8.47 (d, J=2.3 Hz, 1H), 8.23 (d, J=8.8 Hz, 1H), 7.53 (dd, J=8.0, 2.3 Hz, 1H), 7.27 (m, 1H), 7.11 (d, J=7.4 Hz, 1H), 6.87 (dd, J=8.8, 2.3 Hz, 1H), 6.55 (d, J=2.3 Hz, 1H), 6.41-6.31 (m, 1H), 5.40 (m, 3H), 4.52 (d, J=5.1 Hz, 2H), 3.70-3.58 (m, 2H), 2.56 (s, 1H), 1.97-1.71 (m, 5H), 1.59-1.33 (m, 5H), 1.01-0.89 (m, 2H), −0.01 (s, 9H).

Step 5. To a solution of 6-(((5-cyclohexylpyridin-2-yl)methyl)amino)-2-((2-(trimethylsilyl)ethoxy)methyl)isoquinolin-1(2H)-one (304 mg, 0.656 mmol) in THF (5.2 mL) was added at 0 degrees C. methylmagnesium bromide (1.4 M in THF, 1.18 mL, 1.635 mmol) under argon. After 10 minutes at 0 degrees C., powder (R)-1-((perfluorophenyl)sulfonyl)azetidine-2-carbonyl chloride (344 mg, 0.984 mmol) was added at 0 degrees C. The mixture was allowed to reach room temperature and stirred for 1 hour. Cold saturated ammonium chloride was added, followed by water. The mixture was extracted with ethyl acetate (2×). The extract was washed with brine, dried (Na2SO4) and concentrated. Purification by flash column chromatography (6:4 to 4:6 hexane/ethyl acetate) gave (R)—N-((5-cyclohexylpyridin-2-yl)methyl)-N-(1-oxo-2-((2-(trimethylsilyl)ethoxy)methyl)-1,2-dihydroisoquinolin-6-yl)-1-((perfluorophenyl)sulfonyl)azetidine-2-carboxamide (186 mg, 37%). 1H NMR (300 MHz, Chloroform-d) δ 8.46-8.31 (m, 2H), 7.53 (dd, J=8.0, 2.4 Hz, 1H), 7.37 (d, J=2.1 Hz, 1H), 7.33-7.17 (m, 3H), 6.47 (d, J=7.4 Hz, 1H), 5.43 (s, 2H), 5.08-4.88 (m, 3H), 4.14-4.01 (m, 2H), 3.71-3.59 (m, 2H), 2.61-2.45 (m, 2H), 2.42-2.26 (m, 1H), 2.01-1.55 (m, 5H), 1.52-1.35 (m, 5H), 1.04-0.91 (m, 2H) 0.01 (s, 9H).

Step 6. To a solution of (R)—N-((5-cyclohexylpyridin-2-yl)methyl)-N-(1-oxo-2-((2-(trimethylsilyl)ethoxy)methyl)-1,2-dihydroisoquinolin-6-yl)-1-((perfluorophenyl)sulfonyl)azetidine-2-carboxamide (182 mg, 0.234 mmol) in dichloromethane (3.5 mL) was added trifluoroacetic acid (1.15 mL) under argon. The mixture was stirred for 1.5 hours. Additional dichloromethane was added, and the mixture was poured onto cold 10% aqueous sodium bicarbonate to pH 7-8, and extracted with dichloromethane (2×). The extract was washed with additional 10% aqueous sodium bicarbonate, dried (Na2SO4) and concentrated to dryness to obtain crude (R)—N-((5-cyclohexylpyridin-2-yl)methyl)-N-(2-(hydroxymethyl)-1-oxo-1,2-dihydroisoquinolin-6-yl)-1-((perfluorophenyl)sulfonyl)azetidine-2-carboxamide (138 mg, 87%). 1H NMR (300 MHz, Chloroform-d) δ 8.45-8.31 (m, 2H), 7.52 (d, J=8.0 Hz, 1H), 7.40 (s, 1H), 7.34-7.06 (m, 3H), 6.53-6.42 (m, 1H), 5.42 (s, 1.4 H, note: the integration is not up to 2H due to the presence of some des-hydroxymethyl product), 5.08-4.89 (m, 3H), 4.22-3.99 (m, 2H), 2.62-2.44 (m, 1H), 2.43-2.25 (m, 1H), 2.11-1.53 (m, 6H), 1.52-1.13 (m, 5H).

Step 7. To a solution of (R)—N-((5-cyclohexylpyridin-2-yl)methyl)-N-(2-(hydroxymethyl)-1-oxo-1,2-dihydroisoquinolin-6-yl)-1-((perfluorophenyl)sulfonyl)azetidine-2-carboxamide (66.2 mg, 0.098 mmol) in dichloromethane (1.6 mL) was added at 0 degrees C. isopropylamine (0.017 mL, 0.196 mmol). The mixture was stirred at 0 degrees C. for 9 hours. Aqueous 10% acetic acid/sodium acetate (1.5 mL) was added at 0 degrees C., and the mixture was extracted with dichloromethane (2×). The extract was washed with water, dried (Na2SO4) and concentrated. Purification by preparative TLC (4:6 hexane/ethyl acetate with 2% methanol) gave (R)—N-((5-cyclohexylpyridin-2-yl)methyl)-N-(1-oxo-1,2-dihydroisoquinolin-6-yl)-1-((perfluorophenyl)sulfonyl)azetidine-2-carboxamide (45 mg, 62% for 2 steps). 1H NMR (300 MHz, Chloroform-d) δ 11.57 (s, 1H), 8.41-8.31 (m, 2H), 7.52 (dd, J=8.1, 2.3 Hz, 1H), 7.40 (d, J=2.1 Hz, 1H), 7.31-7.18 (m, 2H), 7.17-7.07 (m, 1H), 6.47 (d, J=7.1 Hz, 1H), 5.09-4.89 (m, 3H), 4.20-3.98 (m, 2H), 2.59-2.44 (m, 1H), 2.42-2.24 (m, 1H), 2.02-1.69 (m, 6H), 1.51-1.29 (m, 5H).

### Example 26

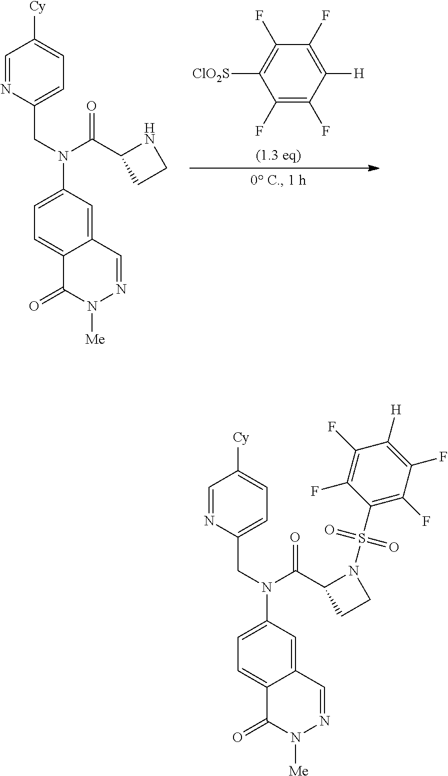

### (R)—N-((5-cyclohexylpyridin-2-yl)methyl)-N-(2-methyl-1-oxo-1,2-dihydrophthalazin-6-yl)-1-((2,3,5,6-tetrafluorophenyl)sulfonyl)azetidine-2-carboxamide

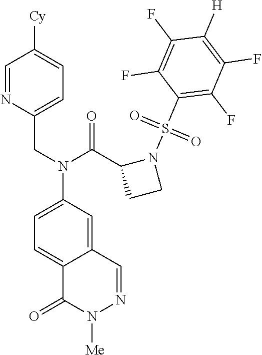

Step 1. Preparation by a similar procedure to Example 7, step 7, starting from (R)—N-((5-cyclohexylpyridin-2-yl)methyl)-N-(2-methyl-1-oxo-1,2-dihydrophthalazin-6-yl)azetidine-2-carboxamide to obtain (R)—N-((5-cyclohexylpyridin-2-yl)methyl)-N-(2-methyl-1-oxo-1,2-dihydrophthalazin-6-yl)-1-((2,3,5,6-tetrafluorophenyl)sulfonyl)azetidine-2-carboxamide (white solid, 58% over 3 steps). 1H NMR (300 MHz, CDCl3) δ 8.43 (d, J=8.4 Hz, 1H), 8.33 (s, 1H), 8.10 (s, 1H), 7.65-7.48 (m, 3H), 7.34-7.19 (m, 2H), 5.09-4.90 (m, 3H), 4.17-4.07 (m, 2H), 3.86 (s, 3H), 2.58-2.45 (m, 1H), 2.43-2.29 (m, 1H), 2.04-1.95 (m, 1H), 1.94-1.71 (m, 6H), 1.49-1.35 (m, 4H). 19F NMR (282 MHz, CDCl3) δ−136.26, −136.54. LRMS (ESI) m/z 644.3 [M+H]+; HRMS (ESI) m/z 644.1986 [M+H]+, 666.1807 [M+Na]+; Purity 100%.

### Example 27

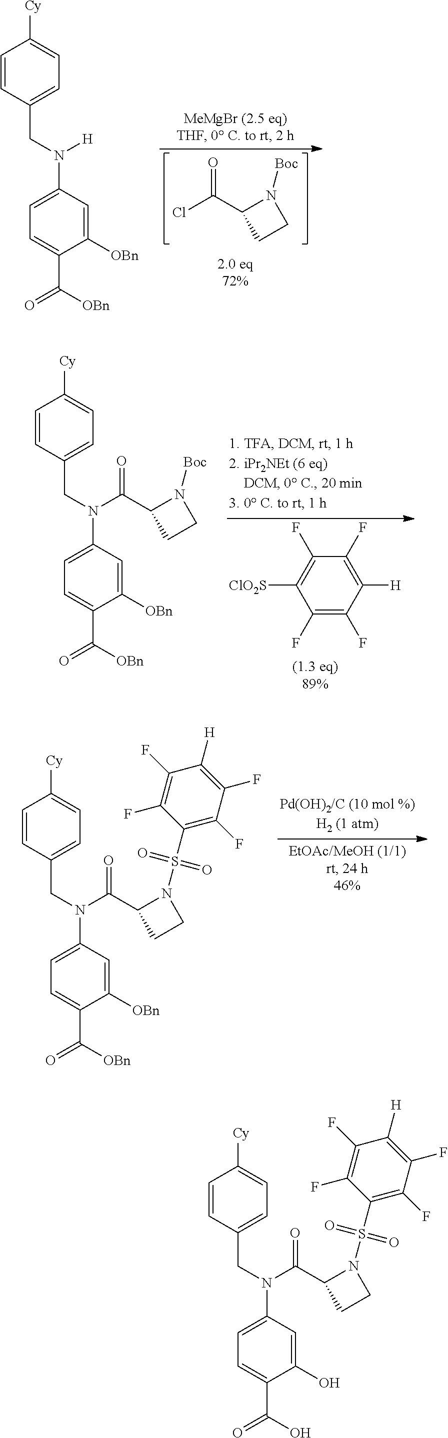

### (R)-4-(N-(4-cyclohexylbenzyl)-1-((2,3,5,6-tetrafluorophenyl)sulfonyl)azetidine-2-carboxamido)-2-hydroxybenzoic acid

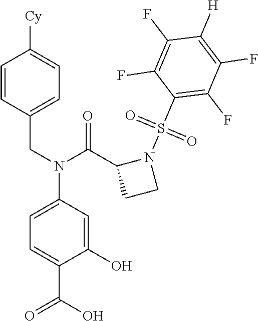

Step 1. To a solution of benzyl 2-(benzyloxy)-4-((4-cyclohexylbenzyl)amino)benzoate (1.0 g, 1.98 mmol, 1.0 equiv) in 20 mL THF was added MeMgBr (3.5 mL, 4.94 mmol, 2.5 equiv, 1.4 M in THF/toluene) at 0 degrees C. under Argon. 10 min later, a solution of tert-butyl (R)-2-(chlorocarbonyl)azetidine-1-carboxylate (2.0 equiv) in 4 mL THF was added to the reaction. The reaction was allowed to warm up to room temperature and stirred for 1 h. Then the reaction was quenched with saturated aqueous ammonium chloride, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 6/1) to provide tert-butyl (R)-2-((3-(benzyloxy)-4-((benzyloxy)carbonyl)phenyl)(4-cyclohexylbenzyl)carbamoyl) azetidine-1-carboxylate as white solid (973 mg, 72%). 1H NMR (300 MHz, CDCl3) δ 7.78 (d, J=8.2 Hz, 1H), 7.42-7.28 (m, 10H), 7.25 (s, 1H), 7.12-7.08 (m, 4H), 6.64 (d, J=7.8 Hz, 1H), 5.33 (s, 2H), 5.11-4.55 (m, 4H), 4.50-4.37 (m, 1H), 4.07-3.96 (m, 1H), 3.75-3.64 (m, 1H), 2.51-2.39 (m, 1H), 2.03-1.66 (m, 8H), 1.44-1.34 (m, 13H).

Step 2. To a solution of tert-butyl (R)-2-((3-(benzyloxy)-4-((benzyloxy)carbonyl)phenyl)(4-cyclohexylbenzyl)carbamoyl)azetidine-1-carboxylate (200 mg, 0.29 mmol, 1.00 equiv) in 3 mL DCM was added TFA (0.9 mL). The reaction was stirred at room temperature for 1 h. Then the reaction was concentrated under reduced pressure, diluted with dry DCE and concentrated again. The residue was dried under high vacuum for 30 min and used directly for the next step.

Step 3. To a solution of the step 2 above residue 2 in 5 mL DCM was added DIPEA (0.29 mL, 1.74 mmol, 6.0 equiv) at 0 degrees C. under Argon. After 10 min, a solution of 2,3,5,6-tetrafluorobenzenesulfonyl chloride (94 mg, 0.38 mmol, 1.3 equiv) in 1 mL DCM was added dropwise under Argon at 0 degrees C. The reaction was stirred at 0 degrees C. for 1 h. Then the reaction was quenched with saturated NH4Cl aq, extracted with DCM (3×). The combined organic extracts were washed with saturated brine, dried over anhydrous sodium sulfate, and concentrated under reduced pressure. The resulting residue was purified by flash chromatography (eluent: hexane/EtOAc 4/1) to provide benzyl (R)-2-(benzyloxy)-4-(N-(4-cyclohexylbenzyl)-1-((2,3,5,6-tetrafluorophenyl)sulfonyl)azetidine-2-carboxamido)benzoate as white solid (207 mg, 89% over 3 steps). 1H NMR (300 MHz, CDCl3) δ 7.81 (d, J=8.2 Hz, 1H), 7.46-7.30 (m, 10H), 7.16-7.10 (m, 2H), 7.09-6.96 (m, 3H), 6.66-6.59 (m, 1H), 6.45 (s, 1H), 5.36 (s, 2H), 5.08-4.78 (m, 3H), 4.74 (s, 2H), 4.09-3.95 (m, 2H), 2.52-2.43 (m, 1H), 2.06-1.98 (m, 1H), 1.88-1.72 (m, 6H), 1.71-1.62 (m, 1H), 1.43-1.34 (m, 4H).

Step 4. Benzyl (R)-2-(benzyloxy)-4-(N-(4-cyclohexylbenzyl)-1-((2,3,5,6-tetrafluorophenyl)sulfonyl) azetidine-2-carboxamido)benzoate (200 mg, 0.25 mmol) and Pd(OH)2/C (20 mg) were dissolved in EtOAc/MeOH (5 mL, 1/1) under hydrogen gas (1 atm). After 24 h, the catalyst was filtered off through a celite pad and washed with ethyl acetate. The combined solvent was concentrated under reduced pressure and the residue was purified by flash chromatography (eluent: DCM/MeOH 10/1) to obtain (R)-4-(N-(4-cyclohexylbenzyl)-1-((2,3,5,6-tetrafluorophenyl)sulfonyl) azetidine-2-carboxamido)-2-hydroxybenzoic acid as white solid (72 mg, 46%). 1H NMR (300 MHz, CD3OD) δ 7.87-7.63 (m, 2H), 7.20-6.97 (m, 4H), 6.55-6.36 (m, 2H), 4.74-4.65 (m, 1H), 3.98 (s, 2H), 2.52-2.40 (m, 1H), 2.35-2.21 (m, 1H), 2.09-1.95 (m, 1H), 1.91-1.66 (m, 5H), 1.51-1.15 (m, 6H). 19F NMR (282 MHz, CD3OD) δ−138.10, −138.83. LRMS (ESI) m/z 621.2 [M+H]+; HRMS (ESI) m/z 621.1681 [M+H]+, 643.1495[M+Na]+; Purity 100%.

### Example 28

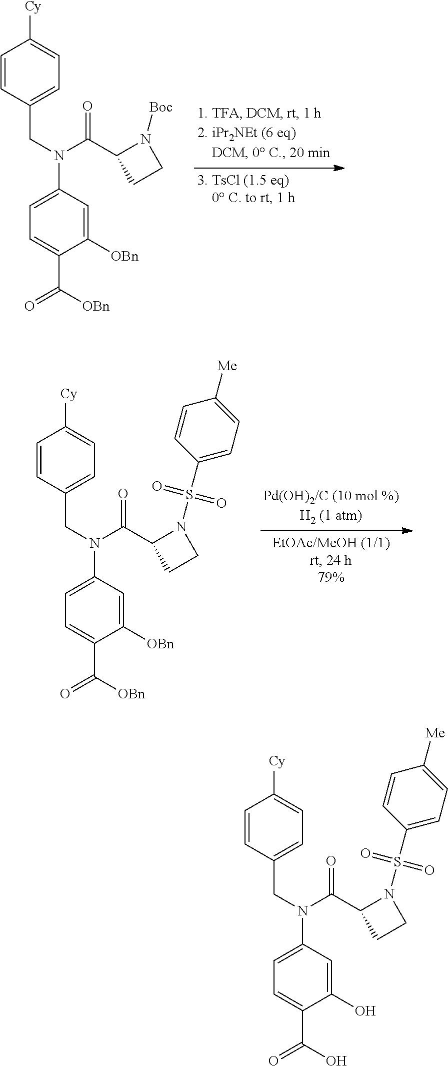

### (R)-4-(N-(4-cyclohexylbenzyl)-1-tosylazetidine-2-carboxamido)-2-hydroxybenzoic acid

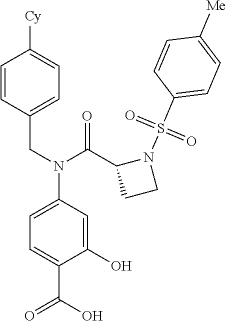

Step 1. To a solution of tert-butyl (R)-2-((3-(benzyloxy)-4-((benzyloxy)carbonyl)phenyl)(4-cyclohexylbenzyl)carbamoyl)azetidine-1-carboxylate (300 mg, 0.44 mmol, 1.00 equiv) in 4 mL DCM was added TFA (1.3 mL). The reaction was stirred at room temperature for 1 h. Then the reaction was concentrated under reduced pressure, diluted with dry DCE and concentrated again. The residue was dried under high vacuum for 30 min and used directly for the next step.

Step 2. To a solution of the above step 1 residue in 9 mL DCM was added DIPEA (0.43 mL, 2.61 mmol, 6.0 equiv) at 0 degrees C. under Argon. After 10 min, p-toluenesulfonyl chloride (124 mg, 0.65 mmol, 1.5 equiv) was added under Argon at 0 degrees C. The reaction was stirred at 0 degrees C. for 1 h. Then the reaction was quenched with saturated NH4Cl aq, extracted with DCM (3×). The combined organic extracts were washed with saturated brine, dried over anhydrous sodium sulfate, and concentrated under reduced pressure. The resulting residue was purified by flash chromatography (eluent: DCM/MeOH 200/1) to provide benzyl (R)-2-(benzyloxy)-4-(N-(4-cyclohexylbenzyl)-1-tosylazetidine-2-carboxamido)benzoate (230 mg), which contained some impurities and was used for the next step without further purification.

Step 3. Benzyl (R)-2-(benzyloxy)-4-(N-(4-cyclohexylbenzyl)-1-tosylazetidine-2-carboxamido)benzoate (200 mg) and Pd(OH)2/C (40 mg) were dissolved in EtOAc/MeOH (5 mL, 1/1) under hydrogen gas (1 atm). After 24 h, the catalyst was filtered off through a celite pad and washed with ethyl acetate. The combined solvent was concentrated under reduced pressure and the residue was purified by flash chromatography (eluent: DCM/MeOH 8/1) to obtain (R)-4-(N-(4-cyclohexylbenzyl)-1-tosylazetidine-2-carboxamido)-2-hydroxybenzoic acid as white solid (120 mg, 79%). 1H NMR (300 MHz, CD3OD) δ 7.89 (s, 1H), 7.57-7.40 (m, 2H), 7.38-7.21 (m, 2H), 7.19-7.04 (m, 4H), 6.53 (m, 2H), 4.88-4.61 (m, 3H), 4.36-4.22 (m, 1H), 3.78-3.65 (m, 1H), 3.53-3.41 (m, 1H), 2.54-2.41 (m, 1H), 2.35 (s, 3H), 1.95-1.57 (m, 6H), 1.52-1.33 (m, 4H). LRMS (ESI) m/z 563.2 [M+H]+; HRMS (ESI) m/z 563.2217 [M+H]+, 5852035+Na1+; Purity 100%.

### Example 29

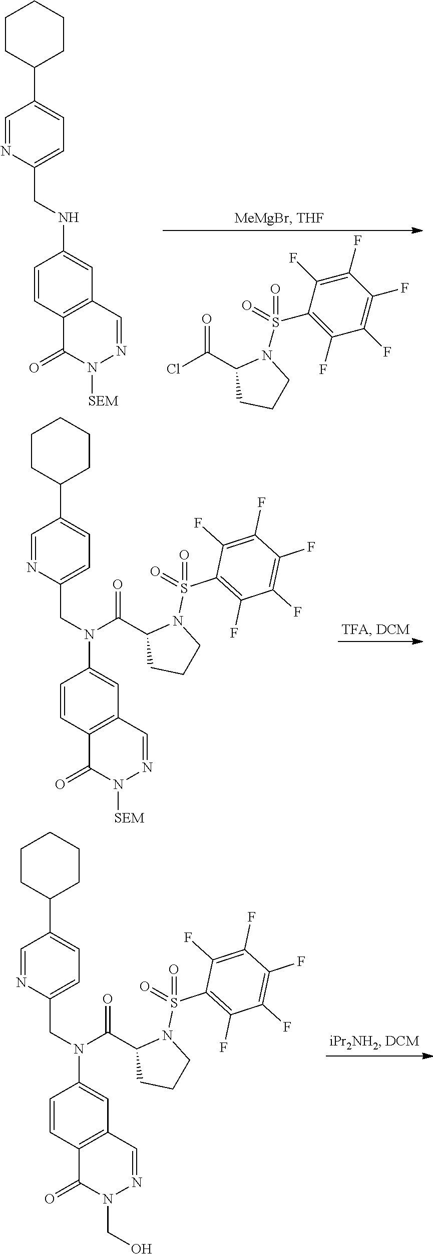

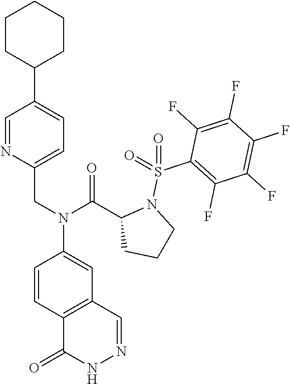 [SMILES: O=C([C@H]1CCCN1S(=O)(=O)c1c(F)c(F)c(F)c(F)c1F)N(Cc1ccc(C2CCCCC2)cn1)c1ccc2c(=O)[nH]ncc2c1]

### (R)—N-((5-cyclohexylpyridin-2-yl)methyl)-N-(1-oxo-1,2-dihydrophthalazin-6-yl)-1-((perfluorophenyl)sulfonyl)pyrrolidine-2-carboxamide

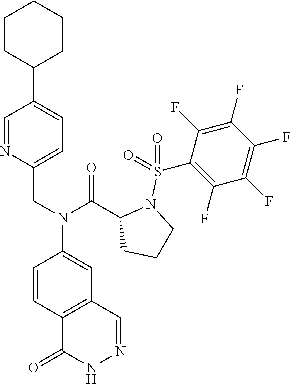 [SMILES: O=C([C@H]1CCCN1S(=O)(=O)c1c(F)c(F)c(F)c(F)c1F)N(Cc1ccc(C2CCCCC2)cn1)c1ccc2c(=O)[nH]ncc2c1]

Step 1. Preparation by a similar procedure to Example 25, step 5, starting from 6-(((5-cyclohexylpyridin-2-yl)methyl)amino)-2-((2-(trimethylsilyl)ethoxy)methyl)phthalazin-1(2H)-one (106 mg, 0.23 mmol) to obtain (R)—N-((5-cyclohexylpyridin-2-yl)methyl)-N-(1-oxo-2-((2-(trimethylsilyl)ethoxy)methyl)-1,2-dihydrophthalazin-6-yl)-1-((perfluorophenyl)sulfonyl)pyrrolidine-2-carboxamide (107 mg, 62% yield). 1H NMR (300 MHz, Chloroform-d) δ 8.49 (d, J=8.3 Hz, 1H), 8.34 (s, 1H), 8.17 (s, 1H), 7.82-7.65 (m, 2H), 7.56-7.46 (m, 1H), 7.25-7.17 (m, 1H), 5.59 (s, 2H), 5.09 (d, J=15.1 Hz, 1H), 4.80 (d, J=15.1 Hz, 1H), 4.65-4.52 (m, 1H), 3.81-3.57 (m, 4H), 2.62-2.43 (m, 1H), 2.16-1.70 (m, 9H), 1.49-1.20 (m, 5H), 1.08-0.95 (m, 2H), 0.02 (s, 9H).

Step 2. Preparation by a similar procedure to Example 25, step 6, starting from (R)—N-((5-cyclohexylpyridin-2-yl)methyl)-N-(1-oxo-2-((2-(trimethylsilyl)ethoxy)methyl)-1,2-dihydrophthalazin-6-yl)-1-((perfluorophenyl)sulfonyl)pyrrolidine-2-carboxamide (104 mg, 0.138 mmol) to obtain crude (R)—N-((5-cyclohexylpyridin-2-yl)methyl)-N-(2-(hydroxymethyl)-1-oxo-1,2-dihydrophthalazin-6-yl)-1-((perfluorophenyesulfonyl)pyrrolidine-2-carboxamide (crude 90 mg), taken as such to next step.

Step 3. Preparation by a similar procedure to Example 25, step 7, starting from crude (R)—N-((5-cyclohexylpyridin-2-yl)methyl)-N-(2-(hydroxymethyl)-1-oxo-1,2-dihydrophthalazin-6-yl)-1-((perfluorophenyl)sulfonyl)pyrrolidine-2-carboxamide (88 mg, 0.134 mmol) to obtain (R)—N-((5-cyclohexylpyridin-2-yl)methyl)-N-(1-oxo-1,2-dihydrophthalazin-6-yl)-1-((perfluorophenyl)sulfonyl)pyrrolidine-2-carboxamide 1H NMR (300 MHz, Chloroform-d) δ 12.07 (s, 1H), 8.50-8.36 (m, 1H), 8.38-8.29 (m, 1H), 8.19-7.98 (m, 1H), 7.78-7.65 (m, 1H), 7.62-7.46 (m, 2H), 7.41-7.19 (m, 1H), 5.23-4.75 (m, 2H), 4.62-4.38 (m, 1H), 3.75-3.58 (m, 2H), 2.64-2.44 (m, 1H), 2.21-1.68 (m, 9H), 1.52-1.16 (m, 5H).

### Example 30

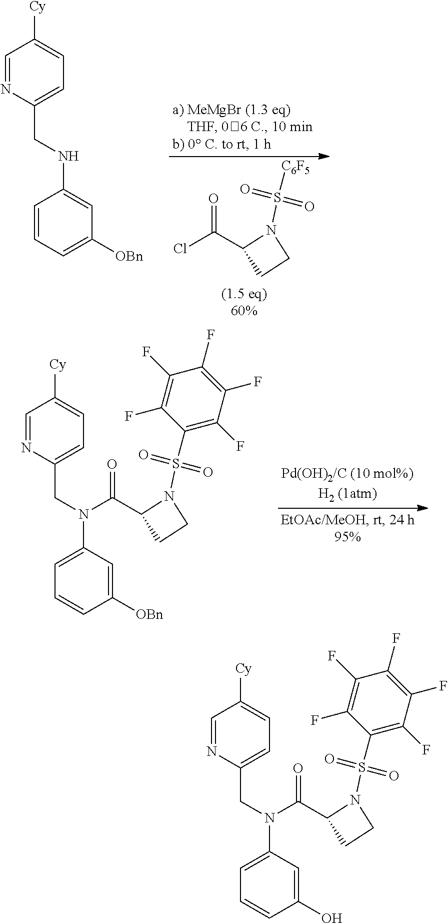

### (R)—N-((5-cyclohexylpyridin-2-yl)methyl)-N-(3-hydroxyphenyl)-1-((perfluoro phenyl)sulfon yl)azetidine-2-carboxamide

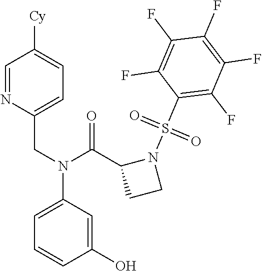

Step 1. To a solution of 3-(benzyloxy)-N-((5-cyclohexylpyridin-2-yl)methyl)aniline (180 mg, 0.48 mmol, 1.00 equiv) in 5 mL THF was added MeMgBr (0.5 mL, 0.63 mmol, 1.3 equiv) at 0 degrees C. under Argon. After 10 min, a solution of (R)-1-((perfluorophenyl)sulfonyl)azetidine-2-carbonyl chloride (253 mg, 0.72 mmol, 1.5 equiv) in 2 mL THF was added to the reaction at 0 degrees C. under Argon. The reaction was allowed to warm up to room temperature and stirred for 1 h. Then the reaction was quenched with saturated NH4Cl aq, and extracted with EtOAc (3×). The combined organic extracts were washed with saturated brine, dried over anhydrous sodium sulfate, and concentrated under reduced pressure. The residue was purified by flash chromatography (eluent: hexane/EtOAc 2/1) to provide (R)—N-(3-(benzyloxy)phenyl)-N-((5-cyclohexylpyridin-2-yl)methyl)-1-((perfluorophenyl) sulfonyl)azetidine-2-carboxamide as white solid (200 mg, 60%). 1H NMR (300 MHz, CDCl3) δ 8.33 (s, 1H), 7.49 (dd, J=8.0, 2.1 Hz, 1H), 7.44-7.29 (m, 5H), 7.25-7.14 (m, 2H), 6.95 (d, J=8.1 Hz, 1H), 6.75-6.66 (m, 2H), 5.06-4.77 (m, 5H), 4.17-4.04 (m, 1H), 4.03-3.95 (m, 1H), 2.55-2.45 (m, 1H), 2.24-2.13 (m, 1H), 1.95-1.69 (m, 7H), 1.45-1.33 (m, 4H).

Step 2. (R)—N-(3-(benzyloxy)phenyl)-N-((5-cyclohexylpyridin-2-yl)methyl)-1-((perfluorophenyl) sulfonyl)azetidine-2-carboxamide (200 mg) and Pd(OH)2/C (40 mg) were dissolved in EtOAc/MeOH (5 mL, 1/1) under hydrogen gas (1 atm). After 24 h, the catalyst was filtered off through a celite pad and washed with ethyl acetate. The combined solvent was concentrated under reduced pressure and the residue was purified by flash chromatography (eluent: DCM/MeOH 100/1) to obtain (R)—N-((5-cyclohexylpyridin-2-yl)methyl)-N-(3-hydroxyphenyl)-1-((perfluorophenyl) sulfonyl)azetidine-2-carboxamide as white solid (165 mg, 95%). 1H NMR (300 MHz, CD3Cl) 6 8.26 (s, 1H), 7.63 (d, J=7.9 Hz, 1H), 7.43 (d, J=7.9 Hz, 1H), 6.99-6.87 (m, 1H), 6.47 (s, 1H), 6.42 (d, J=7.9 Hz, 1H), 6.29 (d, J=7.5 Hz, 1H), 5.17 (d, J=14.7 Hz, 1H), 5.06-4.97 (m, 1H), 4.65 (d, J=14.6 Hz, 1H), 4.18-4.06 (m, 1H), 4.05-3.94 (m, 1H), 2.62-2.48 (m, 1H), 2.30-2.16 (m, 1H), 2.05-1.67 (m, 7H), 1.40 (t, J=10.2 Hz, 4H). LRMS (ESI) m/z 596.2 [M+H]+; HRMS (ESI) m/z 596.1662[M+H]+, 618.1484[M+Na]+; Purity 100%.

### Example 31

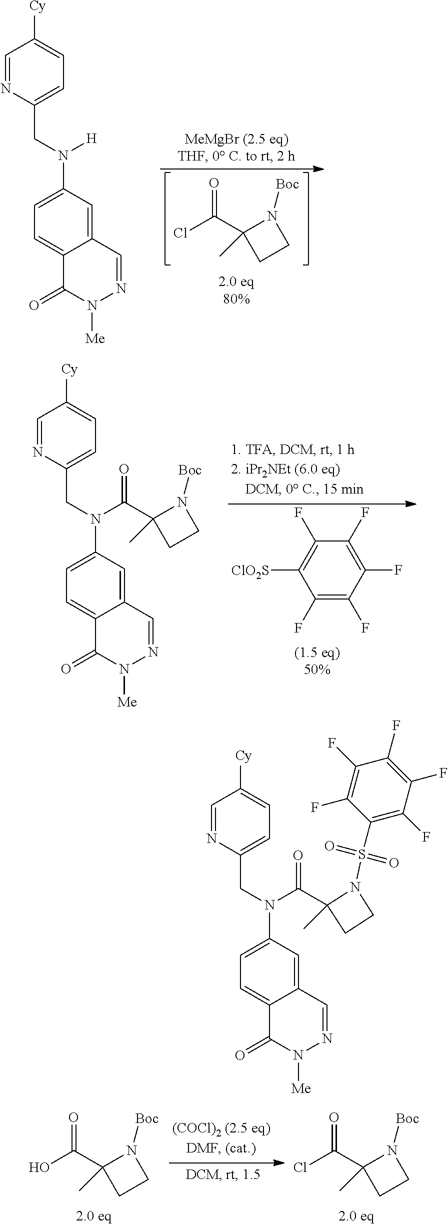

### N-((5-cyclohexylpyridin-2-yl)methyl)-2-methyl-N-(2-methyl-1-oxo-1,2-dihydrophthalazin-6-yl)-1-((perfluorophenyl)sulfonyl)azetidine-2-carboxamide

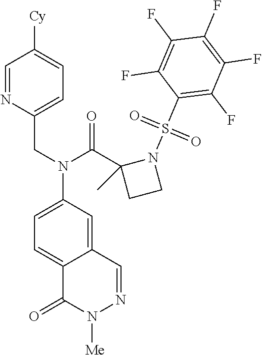

Step 1a. Preparation of the acid chloride: To a solution of 1-(tert-butoxycarbonyl)-2-methylazetidine-2-carboxylic acid (494 mg, 2.30 mmol, 2.0 equiv) in 20 mL DCM was added DMF (2 drops, cat.) and oxalyl chloride (0.24 mL, 2.87 mmol, 2.5 equiv) dropwise under Argon. The reaction was stirred at room temperature for 1.5 h. Then the mixture was concentrated under reduced pressure, diluted with dry DCE and concentrated again. The residue, tert-butyl 2-(chlorocarbonyl)azetidine-1-carboxylate, was dried under high vacuum for 30 min and used directly.

Step 1b. To a solution of 6-(((5-cyclohexylpyridin-2-yl)methyl)amino)-2-methylphthalazin-1(2H)-one (400 mg, 1.15 mmol, 1.0 equiv) in 10 mL THF was added MeMgBr (2.0 mL, 2.87 mmol, 2.5 equiv, 1.4 M in THF/toluene) at 0 degrees C. under Argon. 10 min later, a solution of tert-butyl 2-(chlorocarbonyl)azetidine-1-carboxylate in 5 mL THF was added to the reaction. The reaction was allowed to warm up to room temperature and stirred for 1 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/Acetone 7/2) to provide tert-butyl 2-(((5-cyclohexylpyridin-2-yl)methyl)(2-methyl-1-oxo-1,2-dihydrophthalazin-6-yl)carbamoyl)-2-methylazetidine-1-carboxylate as white solid (500 mg, 80%). 1H NMR (300 MHz, CDCl3) δ 8.40-8.25 (m, 2H), 8.02 (s, 1H), 7.61 (d, J=8.7 Hz, 1H), 7.57 (s, 1H), 7.49 (d, J=8.0 Hz, 1H), 7.34 (d, J=7.9 Hz, 1H), 5.00 (s, 2H), 3.83 (s, 3H), 3.61-3.47 (m, 1H), 3.10-2.92 (m, 1H), 2.80-2.63 (m, 1H), 2.61-2.45 (m, 2H), 1.91-1.55 (m, 10H), 1.50-1.29 (m, 9H), 1.19 (s, 3H).

Step 2. To a solution of tert-butyl 2-(((5-cyclohexylpyridin-2-yl)methyl)(2-methyl-1-oxo-1,2-dihydrophthalazin-6-yl)carbamoyl)-2-methylazetidine-1-carboxylate (153 mg, 0.28 mmol, 1.00 equiv) in 4.5 mL DCM was added TFA (1.5 mL). The reaction was stirred at room temperature for 1 h. Then the reaction was concentrated under reduced pressure, diluted with dry DCE and concentrated again. The residue was dried under high vacuum for 30 min and used directly for the next step.

Step 3. To a solution of the above Step 2 residue in 10 mL DCM was added DIPEA (0.28 mL, 1.68 mmol, 6.0 equiv) at 0 degrees C. under Argon. After 10 min, 2,3,4,5,6-pentafluorobenzenesulfonyl chloride (62 μL, 0.42 mmol, 1.5 equiv) was added dropwise under Argon at 0 degrees C. The reaction was stirred at 0 degrees C. for 1 h. Then the reaction was quenched with saturated NH4Cl aq, extracted with DCM (3×). The combined organic extracts were washed with saturated brine, dried over anhydrous sodium sulfate, and concentrated under reduced pressure. The resulting residue was purified by flash chromatography (eluent: DCM/MeOH 100/1) to provide N-((5-cyclohexylpyridin-2-yl)methyl)-2-methyl-N-(2-methyl-1-oxo-1,2-dihydrophthalazin-6-yl)-1-((perfluorophenyesulfonyl)azetidine-2-carboxamide as white solid (95 mg, 50% over 3 steps). 1H NMR (300 MHz, CDCl3) δ 8.38 (d, J=9.1 Hz, 1H), 8.34 (d, J=2.2 Hz, 1H), 8.04 (s, 1H), 7.59-7.53 (m, 2H), 7.44 (dd, J=8.0, 2.3 Hz, 1H), 7.07 (d, J=8.0 Hz, 1H), 4.95-4.77 (m, 2H), 4.15-4.10 (m, 1H), 3.92-3.86 (m, 1H), 3.83 (s, 3H), 2.71-2.61 (m, 1H), 2.56-2.46 (m, 1H), 1.88-1.78 (m, 7H), 1.56 (s, 3H), 1.44-1.34 (m, 4H). LRMS (ESI) m/z 676.2 [M+H]+; HRMS (ESI) m/z 676.2025[M+H]+, 698.1846 [M+Na]+; Purity 100%.

### Example 32

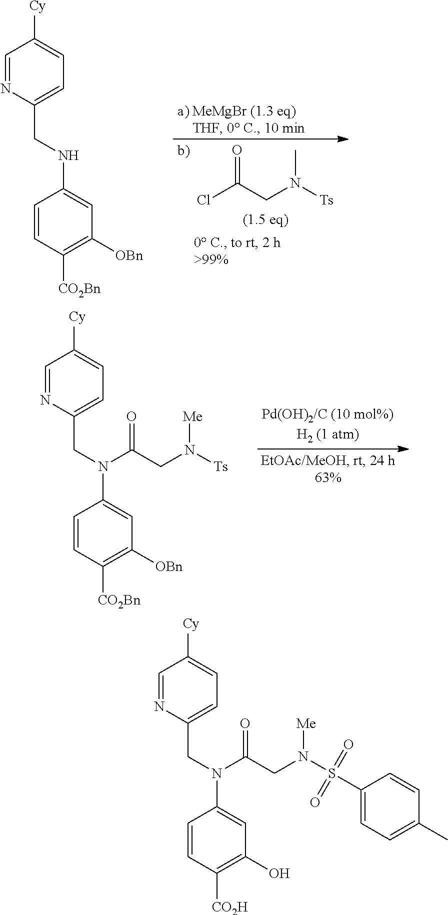

### 4-(N-((5-cyclohexylpyridin-2-yl)methyl)-2-((N,4-dimethylphenyl)sulfonamido)acetamido)-2-hydroxybenzoic acid

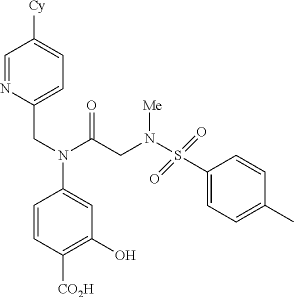

Step 1. To a solution of benzyl 2-(benzyloxy)-4-(((5-cyclohexylpyridin-2-yl)methyl)amino)benzoate (150 mg, 0.30 mmol, 1.00 equiv) in 3 mL THF was added MeMgBr (0.27 mL, 0.39 mmol, 1.3 equiv) at 0 degrees C. under Argon. After 10 min, a solution of N-methyl-N-tosylglycinoyl chloride (116 mg, 0.44 mmol, 1.5 equiv) in 2 mL THF was added to the reaction at 0 degrees C. under Argon. The reaction was allowed to warm up to room temperature and stirred for 1 h. Then the reaction was quenched with saturated NH4Cl aq, and extracted with EtOAc (3×). The combined organic extracts were washed with saturated brine, dried over anhydrous sodium sulfate, and concentrated under reduced pressure. The residue was purified by flash chromatography (eluent: hexane/EtOAc/MeOH 5/1/2%) to provide benzyl 2-(benzyloxy)-4-(N-((5-cyclohexylpyridin-2-yl)methyl)-2-((N,4-dimethylphenyl) sulfonamido)acetamido)benzoate as light yellow oil (224 mg, quantitative). 1H NMR (300 MHz, CDCl3) δ 8.33 (d, J=1.7 Hz, 1H), 7.84 (d, J=8.2 Hz, 1H), 7.61 (d, J=8.2 Hz, 2H), 7.47 (dd, J=8.1, 2.2 Hz, 1H), 7.42-7.27 (m, 10H), 7.23 (d, J=8.2 Hz, 2H), 7.18 (d, J=7.9 Hz, 1H), 6.89 (d, J=1.7 Hz, 1H), 6.81 (dd, J=8.2, 1.8 Hz, 1H), 5.34 (s, 2H), 5.07 (s, 2H), 4.92 (s, 2H), 3.75 (s, 2H), 2.81 (s, 3H), 2.52-2.47 (m, 1H), 2.38 (s, 3H), 1.80 (m, 6H), 1.43-1.33 (m, 4H).

Step 2. Benzyl 2-(benzyloxy)-4-(N-((5-cyclohexylpyridin-2-yl)methyl)-2-((N,4-dimethylphenyl) sulfonamido)acetamido)benzoate (220 mg) and Pd(OH)2/C (22 mg) were dissolved in EtOAc/MeOH (6 mL, 1/1) under hydrogen gas (1 atm). After 24 h, the catalyst was filtered off through a celite pad and washed with ethyl acetate. The combined solvent was concentrated under reduced pressure and the residue was purified by flash chromatography (eluent: DCM/MeOH 10/1) to obtain 4-(N-((5-cyclohexylpyridin-2-yl)methyl)-2-((N,4-dimethylphenyl)sulfonamido)acetamido)-2-hydroxybenzoic acid as light yellow solid (103 mg, 63%). 1H NMR (300 MHz, CD3Cl) δ 8.43 (s, 1H), 7.87-7.44 (m, 5H), 7.24-7.07 (m, 2H), 6.64-6.43 (m, 2H), 5.00 (s, 2H), 3.77 (s, 2H), 2.79 (s, 3H), 2.65-2.50 (m, 1H), 2.36 (s, 3H), 1.99-1.70 (m, 5H), 1.54-1.30 (m, 5H). LRMS (ESI) m/z 552.2 [M+H]+; HRMS (ESI) m/z 552.2147 [M+H]+, 574.1960 1M+Na1+; Purity 97%.

### Example 33

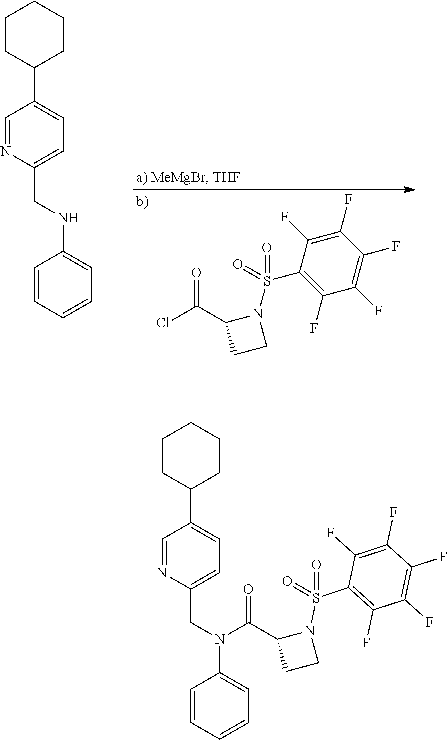 [SMILES: c1ccc(NCc2ccc(C3CCCCC3)cn2)cc1 ; O=C(Cl)[C@H]1CCN1S(=O)(=O)c1c(F)c(F)c(F)c(F)c1F ; O=C([C@H]1CCN1S(=O)(=O)c1c(F)c(F)c(F)c(F)c1F)N(Cc1ccc(C2CCCCC2)cn1)c1ccccc1]

### (R)—N-((5-cyclohexylpyridin-2-yl)methyl)-1-((perfluorophenyl)sulfonyl)-N-phenylazetidine-2-carboxamide

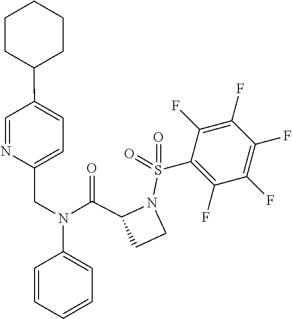 [SMILES: O=C([C@H]1CCN1S(=O)(=O)c1c(F)c(F)c(F)c(F)c1F)N(Cc1ccc(C2CCCCC2)cn1)c1ccccc1]

Step 1. Preparation by a similar procedure to Example 25, step 5, starting from N-((5-cyclohexylpyridin-2-yl)methyl)aniline (see Example 17) to obtain (R)—N-((5-cyclohexylpyridin-2-yl)methyl)-1-((perfluorophenyl)sulfonyl)-N-phenylazetidine-2-carboxamide 1H NMR (300 MHz, Chloroform-d) δ 8.31 (d, J=2.1 Hz, 1H), 7.48 (dd, J=7.9, 2.2 Hz, 1H), 7.40-7.31 (m, 3H), 7.20 (d, J=8.0 Hz, 1H), 7.16-7.08 (m, 2H), 5.07-4.90 (m, 2H), 4.88-4.77 (m, 1H), 4.19-3.94 (m, 2H), 2.57-2.41 (m, 1H), 2.38-2.19 (m, 1H), 2.01-1.71 (m, 6H), 1.52-1.12 (m, 5H). 19F NMR (282 MHz, Chloroform-d) δ−135.35-−136.42 (m), −146.47-−147.72 (m), −159.08-−160.33 (m). HPLC Purity: 99%. HRMS (ESI) m/z=602.1505 [M+Na]+, HRMS (ESI+) calculated for C28H26F5N3O3S: 579.1615, found 579.16139.

### Example 34

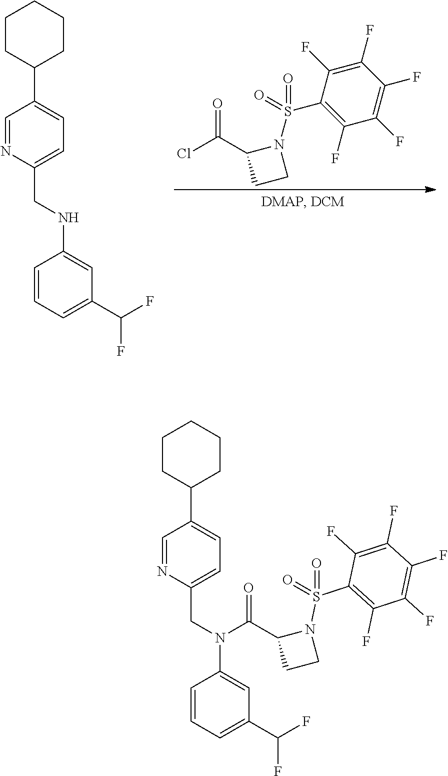 [SMILES: FC(F)c1cccc(NCc2ccc(C3CCCCC3)cn2)c1 ; O=C(Cl)[C@H]1CCN1S(=O)(=O)c1c(F)c(F)c(F)c(F)c1F ; O=C([C@H]1CCN1S(=O)(=O)c1c(F)c(F)c(F)c(F)c1F)N(Cc1ccc(C2CCCCC2)cn1)c1cccc(C(F)F)c1]

### (R)—N-((5-cyclohexylpyridin-2-yl)methyl)-N-(3-(difluoromethyl)phenyl)-1-((perfluorophenyl)sulfonyl)azetidine-2-carboxamide

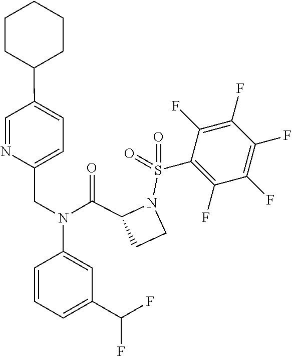 [SMILES: O=C([C@H]1CCN1S(=O)(=O)c1c(F)c(F)c(F)c(F)c1F)N(Cc1ccc(C2CCCCC2)cn1)c1cccc(C(F)F)c1]

Step 1. To a round-bottom flask equipped with a stir bar was added N-((5-cyclohexylpyridin-2-yl)methyl)-3-(difluoromethyl)aniline (see Example 19) (0.319 mmol) in DCM (3 mL) followed by addition of (R)-1-((perfluorophenyl)sulfonyl)azetidine-2-carbonyl chloride (0.351 mmol). The mixture was allowed to stir at 0 degrees C. for 2 minutes before DMAP (0.351 mmol) was added. After stirring for 24 hours at 25 degrees C., the mixture was quenched with water, extracted with dichloromethane, washed with brine, dried with Na2SO4, and concentrated in vacuo. Purification via column chromatography (3:1 hexanes: ethyl acetate) gave (R)—N-((5-cyclohexylpyridin-2-yl)methyl)-N-(3-(difluoromethyl)phenyl)-1-((perfluorophenyl) sulfonyl)azetidine-2-carboxamide (79% yield) as a white solid. 1H NMR (300 MHz, Chloroform-d) δ 8.30 (d, J=2.2 Hz, 1H), 7.53-7.39 (m, 3H), 7.35-7.12 (m, 3H), 6.58 (t, J=56.1 Hz, 1H), 4.97-4.82 (m, 3H), 4.15-3.97 (m, 2H), 2.57-2.40 (m, 1H), 2.38-2.22 (m, 1H), 2.03-1.68 (m, 6H), 1.47-1.14 (m, 5H). 19F NMR (282 MHz, Chloroform-d) δ−111.40 (d, J=56.2 Hz), −111.62 (d, J=56.0 Hz), −135.64-−136.40 (m), −146.66-−147.63 (m), −159.42-−160.30 (m). HPLC Purity=99%. HRMS (ESI) m/z=652.1476 [M+Na]+, HRMS (ESI+) calculated for C29H26F7N3O3S: 629.15831, found 629.15854.

### Example 35

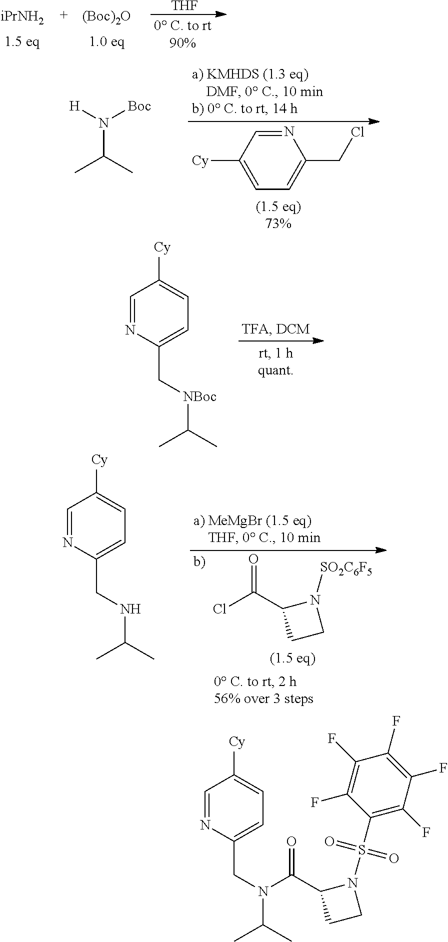

### (R)—N-((5-cyclohexylpyridin-2-yl)methyl)-N-isopropyl-1-((perfluorophenyl)sulfonyl)azetidine-2-carboxamide

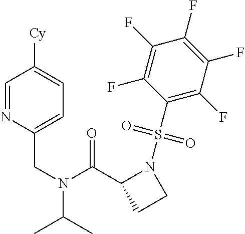

Step 1. To a solution of (Boc)20 (8.9 mL, 38.9 mmol, 1.0 equiv) in 70 mL THF at 0 degrees C. was added iPrNH2 (5 mL, 58.4 mmol, 1.5 equiv) slowly under Argon. The reaction was allowed to warm up to room temperature and stirred at room temperature overnight. Then the reaction was concentrated to obtain tert-butyl isopropylcarbamate as white solid (2.8 g, 90%). 1H NMR (300 MHz, CDCl3) δ 4.34 (s, 1H), 3.83-3.63 (m, 1H), 1.44 (s, 9H), 1.12 (d, J=6.5 Hz, 6H).

Step 2. To a solution of tert-butyl isopropylcarbamate (500 mg, 3.14 mmol, 1.0 equiv) in 15 mL DMF was added KHMDS (4.1 mL, 4.08 mmol, 1.3 equiv, 1.0 M in THF) at 0 degrees C. dropwise under Argon. 10 min late, 2-(chloromethyl)-5-cyclohexylpyridine (9.4 mL, 4.71 mmol, 1.5 equiv, 0.5 M in toluene) was added to the reaction mixture. The reaction was allowed to warm up to room temperature and stirred at room temperature for 20 h. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 10/1) to provide tert-butyl ((5-cyclohexylpyridin-2-yl)methyl)(isopropyl)carbamate as light yellow oil (760 mg, 73%). 1H NMR (300 MHz, CDCl3) δ 8.35 (s, 1H), 7.48 (d, J=8.1 Hz, 1H), 7.18 (d, J=7.7 Hz, 1H), 4.64-4.29 (m, 3H), 2.61-2.46 (m, 1H), 1.97-1.72 (m, 6H), 1.59-1.30 (m, 13H), 1.11 (d, J=6.4 Hz, 6H).

Step 3. To a solution of tert-butyl ((5-cyclohexylpyridin-2-yl)methyl)(isopropyl)carbamate (723 mg, 2.17 mmol) in 20 mL DCM was added TFA (6.5 mL). The reaction was stirred at room temperature for 1 h. Then the reaction was concentrated under reduced pressure, then quenched with saturated NaHCO3 aq. to pH 8. The reaction was extracted with ethyl acetate (3×). The combined organic extracts were washed with saturated brine, dried over anhydrous sodium sulfate, and concentrated under reduced pressure to provide N-((5-cyclohexylpyridin-2-yl)methyl)propan-2-amine as light yellow wet solid (510 mg, quantitative). 1H NMR (300 MHz, CDCl3) δ 8.39 (d, J=1.4 Hz, 1H), 7.50 (dd, J=8.0, 2.0 Hz, 1H), 7.39 (d, J=8.0 Hz, 1H), 5.63 (s, 1H), 4.05 (s, 2H), 3.16-3.01 (m, 1H), 2.58-2.42 (m, 1H), 1.95-1.67 (m, 6H), 1.43-1.33 (m, 4H), 1.28 (d, J=6.4 Hz, 6H).

Step 4. To a solution of N-((5-cyclohexylpyridin-2-yl)methyl)propan-2-amine (183 mg, 0.79 mmol, 1.00 equiv) in 8 mL THF was added MeMgBr (0.84 mL, 1.18 mmol, 1.5 equiv) at 0 degrees C. under Argon. After 10 min, a solution of (R)-1-((perfluorophenyl)sulfonyl)azetidine-2-carbonyl chloride (413 mg, 1.18 mmol, 1.5 equiv) in 2 mL THF was added to the reaction at 0 degrees C. under Argon. The reaction was allowed to warm up to room temperature and stirred for 1 h. Then the reaction was quenched with saturated NH4Cl aq, and extracted with EtOAc (3×). The combined organic extracts were washed with saturated brine, dried over anhydrous sodium sulfate, and concentrated under reduced pressure. The residue was purified by flash chromatography (eluent: hexane/EtOAc 1.5/1) to provide (R)—N-((5-cyclohexylpyridin-2-yl)methyl)-N-isopropyl-1-((perfluorophenyl)sulfonyl)azetidine-2-carboxamide as white solid (240 mg, 56%). There was two rotamers in the proton NMR. 1H NMR (300 MHz, CDCl3) δ 8.41-8.28 (8.38 (d, J=1.8 Hz), 8.30 (d, J=1.7 Hz), 1H), 7.57-7.41 (7.54 (dd, J=8.0, 2.1 Hz), 7.44 (dd, J=8.2, 2.2 Hz), 1H), 7.17-6.97 (7.14 (d, J=8.1 Hz), 7.00 (d, J=8.2 Hz), 1H), 5.47-5.10 (two multiples, 1H), 4.75-3.74 (m, 6H), 2.67-2.25 (m, 3H), 1.98-1.70 (m, 6H), 1.47-1.32 (m, 4H), 1.18-0.98 (1.14 (d, J=6.6 Hz), 1.04 (d, J=6.8 Hz), 6H). LRMS (ESI) m/z 546.2 [M+H]+; HRMS (ESI) m/z 546.1845 [M+H]+, 568.1822 [M+Na]+; Purity 98%.

### Example 36

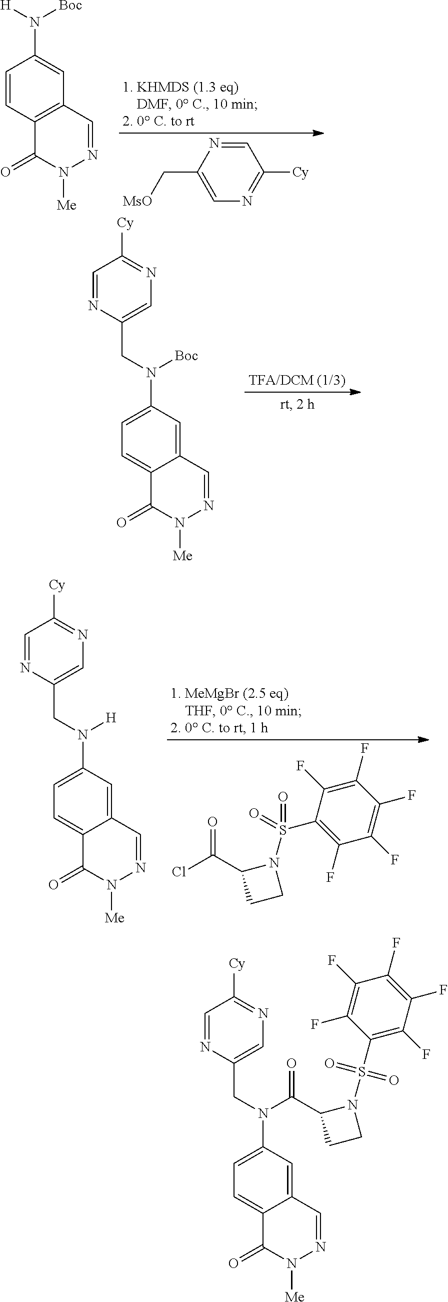

### (R)—N-((5-cyclohexylpyrazin-2-yl)methyl)-N-(2-methyl-1-oxo-1,2-dihydrophthalazin-6-yl)-1-((perfluorophenyl)sulfonyl)azetidine-2-carboxamide

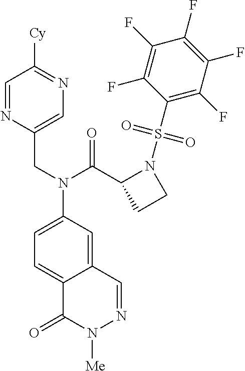

Step 1. To a round-bottom flask equipped with a stir bar was added tert-butyl (2-methyl-1-oxo-1,2-dihydrophthalazin-6-yl)carbamate (see Example 9) (0.977 mmol) in DMF (5.5 mL). The mixture was allowed to stir at 0 degrees C. for 2 minutes before KHMDS (1M in THF, 1.27 mmol) was added. The mixture was allowed to stir at 0 degrees C. for 10 minutes before (5-cyclohexylpyrazin-2-yl)methyl methanesulfonate (1.27 mmol) in DMF (2.5 mL) was added. After stirring for 15.5 hours at 25 degrees C., the reaction was complete. The mixture was quenched with Sat ammonium chloride, extracted with ethyl acetate, washed with brine, dried with Na2SO4, and concentrated in vacuo. Purification via flash chromatography (5:1 hexanes: acetone) gave tert-butyl ((5-cyclohexylpyrazin-2-yl)methyl)(2-methyl-1-oxo-1,2-dihydrophthalazin-6-yl)carbamate (85% yield). 1H NMR (300 MHz, Chloroform-d) δ 8.52 (d, J=1.5 Hz, 1H), 8.40 (d, J=1.5 Hz, 1H), 8.34 (d, J=8.7 Hz, 1H), 8.05 (d, J=0.7 Hz, 1H), 7.76 (dd, J=8.6, 2.2 Hz, 1H), 7.71 (d, J=2.1 Hz, 1H), 5.02 (s, 2H), 3.81 (s, 3H), 2.82-2.64 (m, 1H), 2.01-1.16 (m, 19H). HRMS (ESI) m/z=472.2316 [M+Na]+, HRMS (ESI+) calculated for C25H31N5O3: 449.24269, found 449.24284.

Step 2. Preparation by a similar procedure to Example 7, step 4, starting from tert-butyl ((5-cyclohexylpyrazin-2-yl)methyl)(2-methyl-1-oxo-1,2-dihydrophthalazin-6-yl)carbamate to obtain 6-(((5-cyclohexylpyrazin-2-yl)methyl)amino)-2-methylphthalazin-1(2H)-one. 1H NMR (300 MHz, Chloroform-d) δ 8.53 (d, J=1.5 Hz, 1H), 8.42 (d, J=1.5 Hz, 1H), 8.18 (d, J=8.8 Hz, 1H), 7.94 (s, 1H), 7.07 (dd, J=8.8, 2.4 Hz, 1H), 6.65 (d, J=2.4 Hz, 1H), 5.57 (t, J=5.2 Hz, 1H), 4.54 (d, J=5.2 Hz, 2H), 3.77 (s, 3H), 2.84-2.61 (m, 1H), 2.02-1.70 (m, 5H), 1.63-1.21 (m, 5H). HRMS (ESI) m/z=372.1791 [M+Na]+, HRMS (ESI+) calculated for C20H23N5O: 349.19026, found 349.19006.

Step 3. Preparation by a similar procedure to Example 25, step 5, starting from 6-(((5-cyclohexylpyrazin-2-yl)methyl)amino)-2-methylphthalazin-1(2H)-one to obtain (R)—N-((5-cyclohexylpyrazin-2-yl)methyl)-N-(2-methyl-1-oxo-1,2-dihydrophthalazin-6-yl)-1-((perfluorophenyl)sulfonyl)azetidine-2-carboxamide. 1H NMR (300 MHz, Chloroform-d) δ 8.48-8.40 (m, 2H), 8.35 (d, J=1.5 Hz, 1H), 8.10 (s, 1H), 7.67 (d, J=2.0 Hz, 1H), 7.61 (dd, J=8.4, 2.1 Hz, 1H), 5.14-4.75 (m, 3H), 4.16-3.94 (m, 2H), 3.84 (s, 3H), 2.79-2.57 (m, 1H), 2.40-2.19 (m, 1H), 2.01-1.20 (m, 11H). 19F NMR (282 MHz, Chloroform-d) δ−135.77-−136.06 (m), −146.29-−146.71 (m), −159.16-−159.47 (m). HPLC Purity=100%. HRMS (ESI) m/z=685.1653 [M+Na]+, HRMS (ESI+) calculated for C30H27F5N6O4S: 662.17347, found 662.17509.

### Example 37

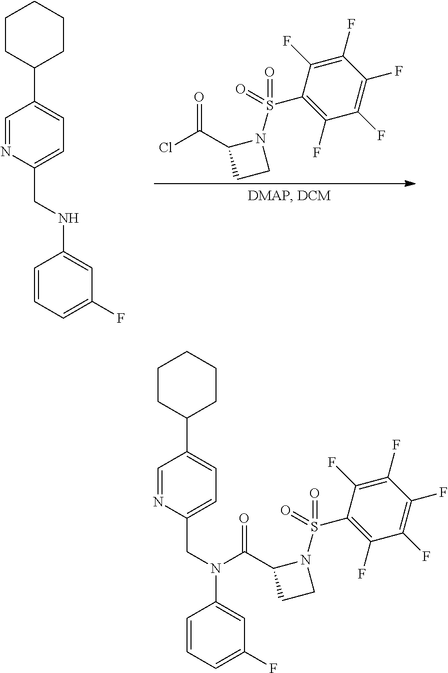 [SMILES: Fc1cccc(NCc2ccc(C3CCCCC3)cn2)c1 ; O=C(Cl)[C@H]1CCN1S(=O)(=O)c1c(F)c(F)c(F)c(F)c1F ; O=C([C@H]1CCN1S(=O)(=O)c1c(F)c(F)c(F)c(F)c1F)N(Cc1ccc(C2CCCCC2)cn1)c1cccc(F)c1]

### (R)—N-((5-cyclohexylpyridin-2-yl)methyl)-N-(3-fluorophenyl)-1-((perfluorophenyl)sulfonyl)azetidine-2-carboxamide

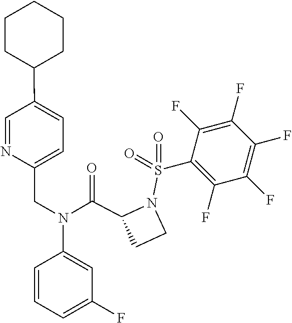 [SMILES: O=C([C@H]1CCN1S(=O)(=O)c1c(F)c(F)c(F)c(F)c1F)N(Cc1ccc(C2CCCCC2)cn1)c1cccc(F)c1]

Step 1. Preparation by a similar procedure to Example 34, step 1, starting from N-((5-cyclohexylpyridin-2-yl)methyl)-3-fluoroaniline (see Example 20) to obtain (R)—N-((5-cyclohexylpyridin-2-yl)methyl)-N-(3-fluorophenyl)-1-((perfluorophenyl)sulfonyl)azetidine-2-carboxamide (46% yield). 1H NMR (300 MHz, Chloroform-d) δ 8.32 (d, J=2.3 Hz, 1H), 7.48 (dd, J=8.1, 2.3 Hz, 1H), 7.39-7.29 (m, 1H), 7.17 (d, J=8.0 Hz, 1H), 7.11-7.01 (m, 1H), 7.00-6.88 (m, 2H), 5.07-4.96 (m, 1H), 4.94-4.80 (m, 2H), 4.20-4.09 (m, 1H), 4.09-3.99 (m, 1H), 2.57-2.43 (m, 1H), 2.39-2.23 (m, 1H), 2.10-1.93 (m, 1H), 1.92-1.71 (m, 5H), 1.51-1.28 (m, 5H). 19F NMR (282 MHz, Chloroform-d) δ−109.48-−109.80 (m), −135.69-−136.08 (m), −145.14-−152.12 (m), −156.90-−163.07 (m). HPLC Purity=100%; HRMS (ESI) m/z=620.1420 [M+Na]+, HRMS (ESI+) calculated.

### Example 38

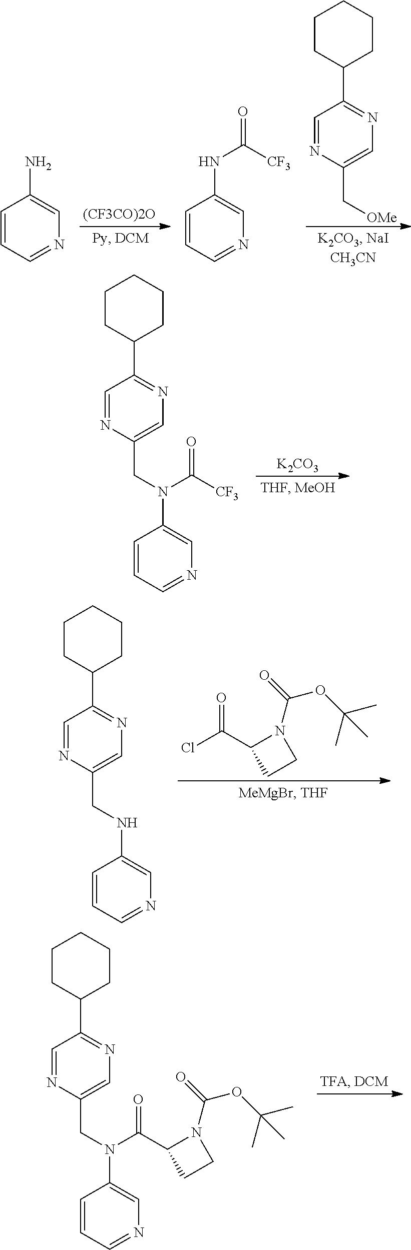

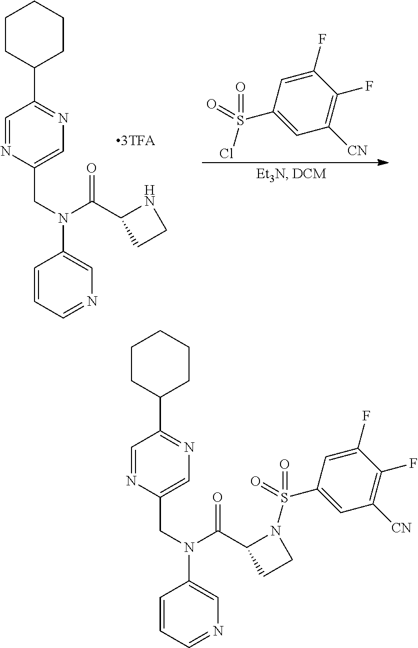 [SMILES: O=C([C@H]1CCN1)N(Cc1cnc(C2CCCCC2)cn1)c1cccnc1 ; N#Cc1cc(S(=O)(=O)Cl)cc(F)c1F ; N#Cc1cc(S(=O)(=O)N2CC[C@@H]2C(=O)N(Cc2cnc(C3CCCCC3)cn2)c2cccnc2)cc(F)c1F]

### (R)-1-((3-cyano-4,5-difluorophenyl)sulfonyl)-N-((5-cyclohexylpyrazin-2-yl)methyl)-N-(pyridin-3-yl)azetidine-2-carboxamide

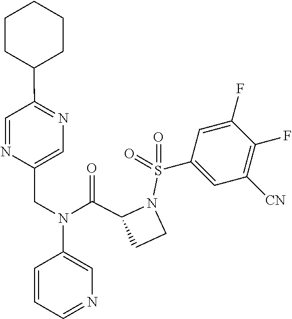 [SMILES: N#Cc1cc(S(=O)(=O)N2CC[C@@H]2C(=O)N(Cc2cnc(C3CCCCC3)cn2)c2cccnc2)cc(F)c1F]

Step 1. Preparation by a similar procedure to Example 5, step 1, starting from 3-aminopyridine (975 mg, 10.4 mmol) to obtain 2,2,2-trifluoro-N-(pyridin-3-yl)acetamide (900 mg, 46%) as white solid. 1H NMR (300 MHz, Chloroform-d) δ 8.70 (dd, J=2.7, 0.8 Hz, 1H), 8.52 (dd, J=4.8, 1.5 Hz, 1H), 8.31 (bs, 1H), 8.21 (ddd, J=8.4, 2.7, 1.5 Hz, 1H), 7.40 (ddt, J=8.4, 4.8, 0.8 Hz, 1H).

Step 2. To (5-cyclohexylpyrazin-2-yl)methyl methanesulfonate (671 mg, 2.48 mmol, for preparation see Example 6, step 1), 2,2,2-trifluoro-N-(pyridin-3-yl)acetamide (396 mg, 2.08 mmol) and sodium iodide (75 mg, 0.38 mmol) was added under argon acetonitrile (33 mL). Potassium carbonate (1.06 g, 7.67 mmol) was added and the mixture was heated at 65° C. for 22 hours. After cooling, aqueous ammonium chloride was added, and the mixture was extracted with EtOAc (2×). The extract was washed with brine, dried (Na2SO4) and concentrated to dryness to obtain crude N-((5-cyclohexylpyrazin-2-yl)methyl)-2,2,2-trifluoro-N-(pyridin-3-yl)acetamide (760 mg), which was taken as such to next step. 1H NMR (300 MHz, Chloroform-d) δ 9.61 (d, J=2.0 Hz, 1H), 8.66 (d, J=1.5 Hz, 1H), 8.45 (d, J=1.5 Hz, 1H), 8.44-8.36 (m, 1H), 8.01 (d, J=5.8 Hz, 1H), 7.66 (dd, J=8.6, 5.8 Hz, 1H), 5.53 (s, 2H), 2.88-2.73 (m, 1H), 2.01-1.73 (m, 5H), 1.67-1.21 (m, 5H).

Step 3. To a solution of crude N-((5-cyclohexylpyrazin-2-yl)methyl)-2,2,2-trifluoro-N-(pyridin-3-yl)acetamide (748.4 mg, 2.05 mmol) in THF (10.5 mL) and methanol (12 mL) was added potassium carbonate (569 mg) under argon. After stirring at room temperature for 2 hours, the mixture was poured onto aqueous ammonium chloride. The mixture was extracted with EtOAc (2×). The extract was washed with brine, dried (Na2SO4) and concentrated. Purification by flash column chromatography (hexane/acetone 1:1 to hexane/acetone 4:6 with 5% methanol) gave recovered starting material (204 mg) and desired N-((5-cyclohexylpyrazin-2-yl)methyl)pyridin-3-amine (70 mg) as a yellow solid. 1H NMR (300 MHz, Chloroform-d) δ 8.55 (d, J=1.5 Hz, 1H), 8.45 (d, J=1.5 Hz, 1H), 8.16 (d, J=2.9 Hz, 1H), 8.02 (dd, J=4.7, 1.4 Hz, 1H), 7.14 (ddd, J=8.3, 4.7, 0.7 Hz, 1H), 6.99 (ddd, J=8.3, 2.9, 1.4 Hz, 1H), 4.82 (br s, 1H), 4.49 (s, 2H), 2.85-2.69 (m, 1H), 2.01-1.73 (m, 5H), 1.69-1.21 (m, 5H).

Step 4. To a solution of N-((5-cyclohexylpyrazin-2-yl)methyl)pyridin-3-amine (53.9 mg, 0.201 mmol) in THF (1.5 mL) was added at 0° C. under argon methylmagnesium bromide (1.4 M, 0.36 mL). After stirring for 10 minutes at 0° C., a solution of tert-butyl (R)-2-(chlorocarbonyl)azetidine-1-carboxylate [prepared from (R)-1-(tert-butoxycarbonyl)azetidine-2-carboxylic acid (80.8 mg, 0.402 mmol), oxalyl chloride (0.044 mL), DMF (one drop) in DCM (2.4 mL); and after concentrating to dryness, the resulting acid chloride was dissolved in THF (1.5 mL) was added at 0° C. The mixture was allowed to reach room temperature and stirred for one hour. Cold aqueous ammonium chloride was added and the mixture was extracted with ethyl acetate (2×). The extract was washed with brine, dried (Na2SO4) and concentrated. Purification by flash column chromatography (1:1 hexane/acetone) gave tert-butyl (R)-2-(((5-cyclohexylpyrazin-2-yl)methyl)(pyridin-3-yl)carbamoyl)azetidine-1-carboxylate (46 mg, 51% yield) as a yellow oil. 1H NMR (300 MHz, Chloroform-d) δ 8.66-8.57 (m, 1H), 8.55-8.47 (m, 1H), 8.36 (s, 1H), 7.79-7.62 (m, 1H), 7.43-7.32 (m, 1H), 7.30-7.26 (m, 1H), 5.40-4.63 (m, 2H), 4.59-4.46 (s, 1H), 4.12-3.99 (m, 1H), 3.77 (q, J=7.4 Hz, 1H), 2.81-2.66 (m, 1H), 2.29-2.09 (m, 2H), 1.99-1.71 (m, 5H), 1.56-1.24 (m, 14H).

Step 5. To a solution of tert-butyl (R)-2-(((5-cyclohexylpyrazin-2-yl)methyl)(pyridin-3-yl)carbamoyl)azetidine-1-carboxylate (45.9 mg, 0.102 mmol) in DCM (1 mL) was added TFA (0.5 mL) under argon, After stirring for one hour at room temperature, the mixture was concentrated to dryness. During concentration dichloroethane (2×) was added to help remove TFA. The crude (R)—N-((5-cyclohexylpyrazin-2-yl)methyl)-N-(pyridin-3-yl)azetidine-2-carboxamide TFA salt (82 mg, approximate 3 TFA molecules are complexed) was taken as such for next step. 1H NMR (300 MHz, Chloroform-d) δ 8.86-8.73 (m, 1H), 8.68-8.61 (m, 1H), 8.59-8.53 (m, 1H), 8.21-8.09 (m, 1H), 7.79-7.69 (m, 1H), 7.31-7.23 (m, 1H), 5.30-5.07 (m, 2H), 4.92 (d, J=15.5 Hz, 1H), 4.27-4.08 (m, 1H), 4.06-3.92 (m, 1H), 2.91-2.74 (m, 1H), 2.72-2.57 (m, 1H), 2.53-2.34 (m, 1H), 2.02-1.74 (m, 5H), 1.64-1.21 (m, 5H).

Step 6. To a solution of crude (R)—N-((5-cyclohexylpyrazin-2-yl)methyl)-N-(pyridin-3-yl)azetidine-2-carboxamide TFA salt (68 mg, 0.098 mmol) in DCM (1.3 mL) were added triethylamine (0.062 mL, 0.441 mmol), followed by 3-cyano-4,5-difluorobenzenesulfonyl chloride (33 mg, 0.103 mmol) under argon. After stirring at room temperature for 2.5 h, water was added, and the mixture was extracted with DCM (1×). The extract was dried (Na2SO4) and concentrated. Purification by preparative TLC (7:3 hexane, acetone) gave (R)-1-((3-cyano-4,5-difluorophenyl)sulfonyl)-N-((5-cyclohexylpyrazin-2-yl)methyl)-N-(pyri din-3-yl)azetidine-2-carboxamide (44.5 mg) as a white foam. 1H NMR (300 MHz, Chloroform-d) δ 8.67 (dd, J=4.7, 1.5 Hz, 1H), 8.55-8.47 (m, 2H), 8.40 (d, J=1.5 Hz, 1H), 8.24-8.02 (m, 2H), 7.71 (dt, J=8.1, 1.8 Hz, 1H), 7.42 (dd, J=8.1, 4.7 Hz, 1H), 5.15 (d, J=15.4 Hz, 1H), 5.00-4.80 (m, 2H), 4.09-3.95 (m, 1H), 3.72-3.59 (m, 1H), 2.84-2.64 (m, 1H), 2.46-2.27 (m, 1H), 2.01-1.17 (m, 11H). 19F NMR (282 MHz, Chloroform-d) δ −122.65, −129.78.

### Example 39

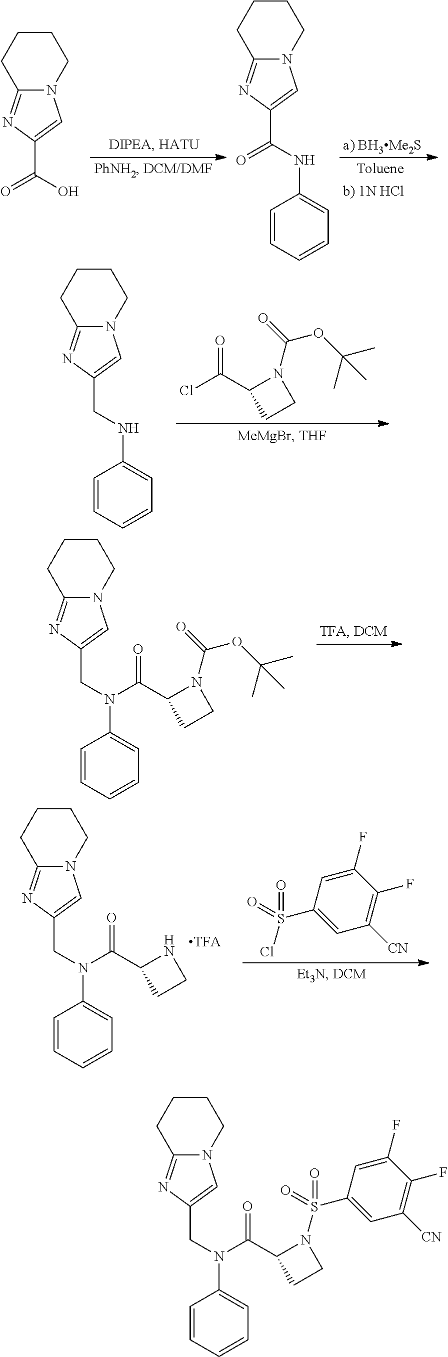

### (R)-1-((3-cyano-4,5-difluorophenyl)sulfonyl)-N-phenyl-N-((5,6,7,8-tetrahydro imidazo[1,2-a]pyridin-2-yl)methyl)azetidine-2-carboxamide

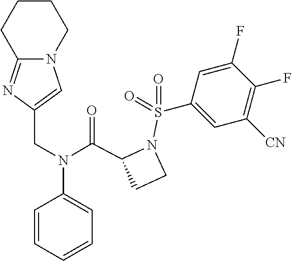 [SMILES: N#Cc1cc(S(=O)(=O)N2CC[C@@H]2C(=O)N(Cc2cn3c(n2)CCCC3)c2ccccc2)cc(F)c1F]

Step 1. A suspension of 5,6,7,8-tetrahydroimidazo[1,2-a]pyridine-2-carboxylic acid (208 mg, 1.25 mmol) in dichloromethane (8 mL) and DMF (8 mL) was sonicated for 5-10 minutes, then DIPEA (0.41 mL) was added. After stirring for 10 minutes, the mixture was cooled to 0° C. and HATU (516 mg, 1.36 mmol) was added. The mixture was allowed to reach room temperature and stirred for 1.5 hours. Aniline (0.127 mL, 1.39 mmol) was added, and the mixture was stirred for 20 hours. The mixture was poured onto aqueous sodium bicarbonate, and was extracted with dichloromethane (2×). The extract was washed with water (2×), dried (Na2SO4) and concentrated. Purification by flash column chromatography (1:1 hexane/acetone) gave N-phenyl-5,6,7,8-tetrahydroimidazo [1,2-a]pyridine-2-carboxamide (191 mg, 63% yield) of a white solid. 1H NMR (300 MHz, Chloroform-d) δ 8.89 (bs, 1H), 7.77-7.66 (m, 2H), 7.53 (s, 1H), 7.42-7.27 (m, 2H), 7.17-7.05 (m, 1H), 4.05 (t, J=5.6 Hz, 2H), 2.91 (t, J=6.1 Hz, 2H), 2.12-1.93 (m, 4H).

Step 2. To a solution of N-phenyl-5,6,7,8-tetrahydroimidazo[1,2-a]pyridine-2-carboxamide (187.3 mg, 0.776 mmol) in toluene (7.5 mL) was added borane dimethyl sulfide complex (0.297 mL, 3.13 mL) under argon. The mixture was heated at 110° C. (oil bath temperature) for 16 hours. After cooling, 1N HCl (20 mL) was added and heated at 100° C. for 30 minutes. The mixture was cooled to room temperature, and 1M KOH was added to basic pH. The mixture was extracted with dichloromethane (2×). The extract was washed with water, dried (Na2SO4), and concentrated. Purification by flash column chromatography (95:5 DCM/MeOH) gave N-((5,6,7,8-tetrahydroimidazo[1,2-a]pyridin-2-yl)methyl)aniline (89 mg, 50% yield) as a white solid. 1H NMR (300 MHz, Chloroform-d) δ 7.35-7.12 (m, 3H), 6.78-6.64 (m, 3H), 4.24 (s, 2H), 3.97-3.86 (m, 2H), 2.87 (t, J=6.0 Hz, 2H), 2.06-1.86 (m, 4H).

Step 3. Preparation by a similar procedure to Example 38, step 4, starting from N-((5,6,7,8-tetrahydroimidazo[1,2-a]pyridin-2-yl)methyl)aniline (83.8 mg, 0.369 mmol) to obtain tert-butyl (R)-2-(phenyl ((5,6,7,8-tetrahydroimidazo[1,2-a]pyridin-2-yl)methyl)carbamoyl)azetidine-1-carboxylate (80 mg, 53% yield) as a white foam. 1H NMR (300 MHz, Chloroform-d) δ 7.45-7.11 (m, 5H), 6.93 (s, 1H), 4.88 (d, J=14.7 Hz, 1H), 4.79-4.60 (m, 1H), 4.59-4.45 (m, 1H), 4.13-4.01 (m, 1H), 3.92 (t, J=5.8, 2H), 3.87-3.67 (m, 1H), 2.81 (t, J=6.0 Hz, 2H), 2.22-2.05 (m, 1H), 2.03-1.70 (m, 5H), 1.41 (s, 9H).

Step 4. Preparation by a similar procedure to Example 4, step 4, starting from tert-butyl (R)-2-(phenyl ((5,6,7,8-tetrahydroimidazo[1,2-a]pyridin-2-yl)methyl)carbamoyl)azetidine-1-carboxylate (77 mg, 0.188 mmol) to obtain (R)—N-phenyl-N-((5, 6,7,8-tetrahydroimidazo[1,2-a]pyridin-2-yl)methyl) azetidine-2-carboxamide TFA salt (74 mg, 93% yield). 1H NMR (300 MHz, Chloroform-d) δ 7.45-7.33 (m, 3H), 7.19 (m, 2H), 6.71 (bs, 1H), 4.95-4.58 (m, 3H), 3.90 (t, J=5.5 Hz, 2H), 3.82-3.57 (m, 2H), 2.91-2.74 (m, 2H), 2.56-2.38 (s, 1H), 2.03-1.82 (m, 5H).

Step 5. Preparation by a similar procedure to Example 6, step 5, starting from (R)—N-phenyl-N-((5, 6,7,8-tetrahydroimidazo[1,2-a]pyridin-2-yl)methyl)azetidine-2-carboxamide TFA salt (77 mg, 0.188 mmol) to obtain (R)-1-((3-cyano-4,5-difluorophenyl)sulfonyl)-N-phenyl-N-((5,6,7,8-tetrahydroimidazo[1,2-a]pyridin-2-yl)methyl)azetidine-2-carboxamide (74 mg, 93%) as a yellow foam. 1H NMR (300 MHz, Chloroform-d) δ 8.30-8.14 (m, 1H), 8.06 (d, J=4.9 Hz, 1H), 7.53-7.32 (m, 3H), 7.27-7.15 (m, 2H), 6.77 (s, 1H), 4.91 (t, J=8.4 Hz, 1H), 4.86-4.66 (m, 2H), 4.06-3.79 (m, 3H), 3.74-3.55 (m, 1H), 2.81 (t, J=6 Hz, 2H), 2.39-2.20 (m, 1H), 2.15-1.66 (m, 5H). 19F NMR (282 MHz, Chloroform-d) δ−123.09, −130.00.

### Example 40

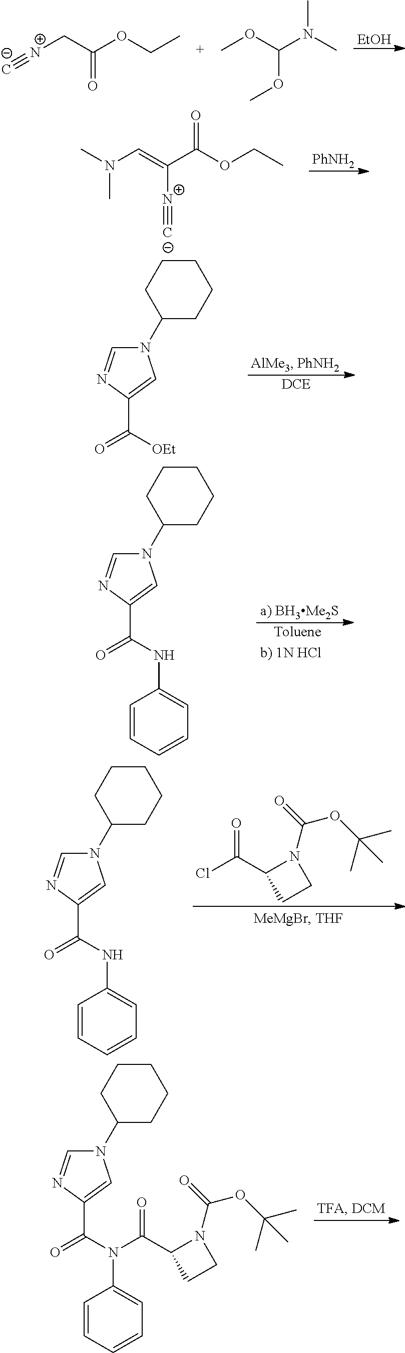

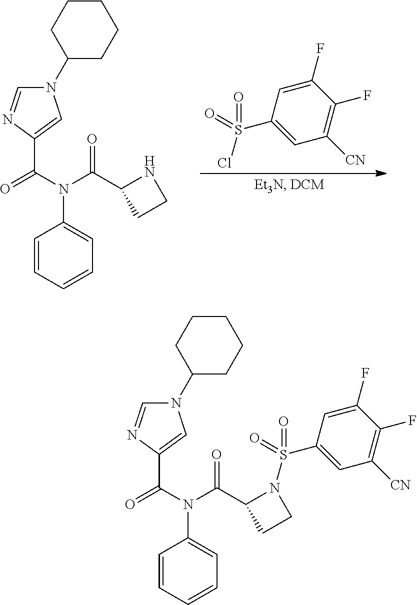 [SMILES: O=C(c1cn(C2CCCCC2)cn1)N(C(=O)[C@H]1CCN1)c1ccccc1 ; N#Cc1cc(S(=O)(=O)Cl)cc(F)c1F ; N#Cc1cc(S(=O)(=O)N2CC[C@@H]2C(=O)N(C(=O)c2cn(C3CCCCC3)cn2)c2ccccc2)cc(F)c1F]

### (R)-1-((3-cyano-4,5-difluorophenyl)sulfonyl)-N-((1-cyclohexyl-1H-imidazol-4-yl)methyl)-N-phenylazetidine-2-carboxamide

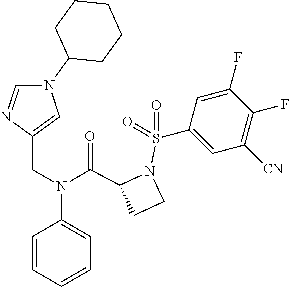 [SMILES: N#Cc1cc(S(=O)(=O)N2CC[C@@H]2C(=O)N(Cc2cn(C3CCCCC3)cn2)c2ccccc2)cc(F)c1F]

Step 1. To a solution of ethyl 2-isocyanoacetate (2.05 g, 18.1 mmol) in ethanol (18 mL) was added at 0° C. N,N-dimethylformamide dimethyl acetal (4.6 mL, 36.2 mmol) under argon. The mixture was allowed to reach room temperature, and stirred for 24 hours. The mixture was concentrated, and purified by flash column chromatography (8:2 hexane/ethyl acetate) to obtain ethyl 3-(dimethylamino)-2-isocyanoacrylate (1.46 g, 48% yield). 1H NMR (500 MHz, Chloroform-d) δ 7.20 (s, 1H), 4.23 (q, J=7.1 Hz, 2H), 3.47-2.97 (m, 6H), 1.32 (t, J=7.1 Hz, 3H).

Step 2. A mixture of ethyl 3-(dimethylamino)-2-isocyanoacrylate (1.41 g, 8.38 mmol) and cyclohexylamine (2.87 mL, 25.1 mmol) was heated under argon at 70° C. for 2 hours. After cooling to room temperature, ethyl acetate (ca. 100 mL) was added, and the mixture was washed with pH 2 buffer. The aqueous phase was extracted with EtOAc (1×). The combined organic phase was washed with brine, dried (Na2SO4), and concentrated. Purification by flash column chromatography (1:1 hexane/EtOAc to 3:7 hexane/EtOAc with 15% MeOH) gave ethyl 1-cyclohexyl-1H-imidazole-4-carboxylate (1.22 g, 66% yield). 1H NMR (500 MHz, Chloroform-d) δ 7.72 (s, 1H), 7.69 (s, 1H), 4.38 (q, J=7.1 Hz, 2H), 3.98 (tt, J=11.8, 3.8 Hz, 1H), 2.15 (d, J=12.6 Hz, 2H), 1.94 (d, J=13.9 Hz, 2H), 1.78 (d, J=13.4 Hz, 1H), 1.65 (qd, J=12.5, 3.5 Hz, 2H), 1.51-1.35 (m, 5H), 1.27 (qt, J=12.9, 3.6 Hz, 1H).

Step 3. To a solution of aniline (316 mg, 3.39 mmol) in 1,2-dichloroethane (3.4 mL) was added under argon 2M trimethylaluminum in toluene (3.4 mL, 6.8 mmol). The mixture was stirred at room temperature for 30 minutes. A solution of ethyl 1-cyclohexyl-1H-imidazole-4-carboxylate (502 mg, 2.26 mmol) in 1,2-dichloroethane (3.4 mL) was added, and the mixture was heated at 82° C. for 6 hours. After cooling, ice/water was added. The mixture was stirred for 10-15 minutes. 1M potassium hydroxide was added, and the mixture was extracted with dichloromethane (2×). The extract was washed with water, dried (Na2SO4) and concentrated. Purification by flash column chromatography (1:1 hexane/EtOAc) gave 1-cyclohexyl-N-phenyl-1H-imidazole-4-carboxamide (565 mg, 93% yield) as a white solid. 1H NMR (300 MHz, Chloroform-d) δ 8.95 (bs, 1H), 7.72 (dt, J=8.5, 1.2 Hz, 3H), 7.53 (d, J=1.5 Hz, 1H), 7.43-7.32 (m, 2H), 7.18-7.06 (m, 1H), 3.98 (tt, J=11.8, 3.8 Hz, 1H), 2.22-2.11 (m, 2H), 2.02-1.89 (m, 3H), 1.85-1.58 (m, 2H), 1.55-1.18 (m, 3H).

Step 4. Preparation by a similar procedure to Example 39, step 2, starting from 1-cyclohexyl-N-phenyl-1H-imidazole-4-carboxamide (557.6, 2.07 mmol) to obtain N-((1-cyclohexyl-1H-imidazol-4-yl)methyl)aniline (109 mg, 21% yield). 1H NMR (300 MHz, Chloroform-d) δ 7.53 (d, J=1.3 Hz, 1H), 7.33-7.13 (m, 2H), 6.90 (d, J=1.3 Hz, 1H), 6.79-6.64 (m, 3H), 4.26 (d, J=0.8 Hz, 2H), 3.88 (tt, J=12.0, 3.9 Hz, 1H), 2.16-2.07 (m, 2H), 1.97-1.86 (m, 2H), 1.82-1.71 (m, 1H), 1.70-1.53 (m, 3H), 1.50-1.32 (m, 2H), 1.32-1.20 (m, 1H).

Step 5. Preparation by a similar procedure to Example 38, step 4, starting from N-((1-cyclohexyl-1H-imidazol-4-yl)methyl)aniline (109 mg, 0.427 mmol) to obtain tert-butyl (R)-2-(((1-cyclohexyl-1H-imidazol-4-yl)methyl)(phenyl)carbamoyl)azetidine-1-carboxylate (183 mg, 98% yield). 1H NMR (300 MHz, Chloroform-d) δ 7.44-7.24 (m, 4H), 7.24-7.03 (m, 3H), 4.87 (d, J=14.9 Hz, 1H), 4.75 (q, J=14.9 Hz, 1H), 4.58-4.40 (m, 1H), 4.07-3.97 (m, 1H), 3.98-3.65 (m, 3H), 2.58-2.38 (m, 1H), 2.23-1.98 (m, 4H), 1.97-1.20 (m, 7H), 1.42 (s, 9H).

Step 6. Preparation by a similar procedure to Example 38, step 5, starting from tert-butyl (R)-2-(((1-cyclohexyl-1H-imidazol-4-yl)methyl)(phenyl)carbamoyl)azetidine-1-carboxylate (182.7 mg, 0.416 mmol) to obtain (R)—N-((1-cyclohexyl-1H-imidazol-4-yl)methyl)-N-phenylazetidine-2-carboxamide (110.9 mg, 79% yield). 1H NMR (300 MHz, Chloroform-d) δ 7.51 (d, J=1.4 Hz, 1H), 7.42-7.31 (m, 3H), 7.06 (dd, J=6.6, 3.1 Hz, 2H), 6.83 (d, J=1.4 Hz, 1H), 4.87 (d, J=14.6 Hz, 1H), 4.78 (d, J=14.6 Hz, 1H), 4.67 (dd, J=9.2, 7.2 Hz, 1H), 3.92-3.75 (m, 3H), 2.52-2.36 (m, 1H), 2.26-2.12 (m, 1H), 2.11-1.99 (m, 2H), 1.96-1.83 (m, 2H), 1.79-1.68 (m, 1H), 1.68-1.49 (m, 2H), 1.48-1.30 (m, 2H), 1.29-1.16 (m, 1H).

Step 7. Preparation by a similar procedure to Example 38, step 6, starting from (R)—N-((1-cyclohexyl-1H-imidazol-4-yl)methyl)-N-phenylazetidine-2-carboxamide TFA salt (110.9 mg, 0.245 mmol as free base) to obtain only the pure fraction from flash chromatography column (R)-1-((3-cyano-4,5-difluorophenyl)sulfonyl)-N-((1-cyclohexyl-1H-imidazol-4-yl)methyl)-N-phenylazetidine-2-carboxamide (33.4 mg, 25% yield). 1H NMR (300 MHz, Chloroform-d) δ 8.20 (ddd, J=9.2, 7.0, 2.2 Hz, 1H), 8.06 (dt, J=4.5, 2.2 Hz, 1H), 7.52-7.33 (m, 4H), 7.14 (dd, J=6.5, 2.9 Hz, 2H), 6.92 (s, 1H), 5.03-4.70 (m, 3H), 4.01-3.75 (m, 2H), 3.65 (ddd, J=10.9, 7.4, 4.0 Hz, 1H), 2.30 (p, J=8.4 Hz, 1H), 2.13-2.02 (m, 1H), 1.97-1.82 (m, 2H), 1.82-1.68 (m, 2H), 1.69-1.49 (m, 3H), 1.51-1.31 (m, 2H), 1.37-1.16 (m, 1H). 19F NMR (282 MHz, Chloroform-d) δ−123.09, −130.00.

### Example 41

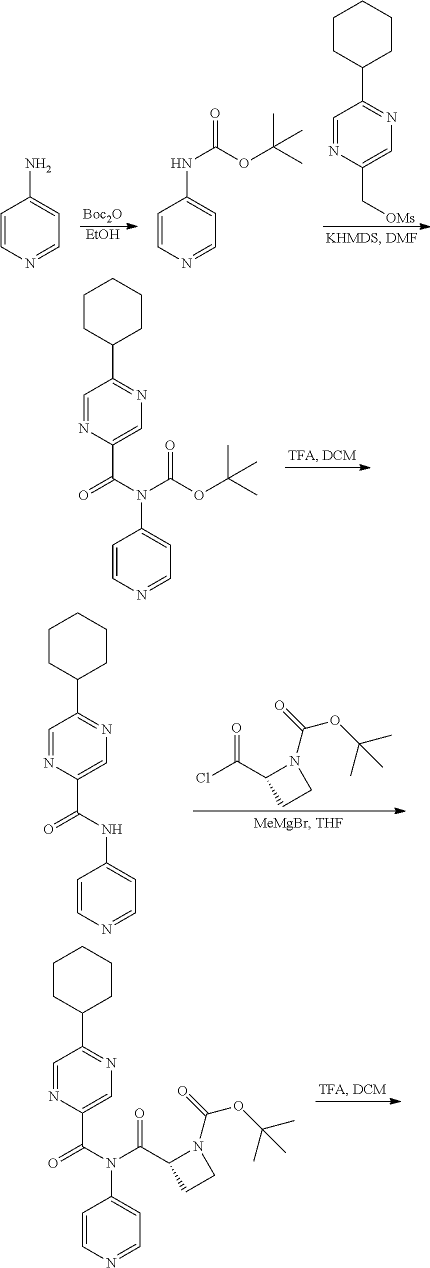

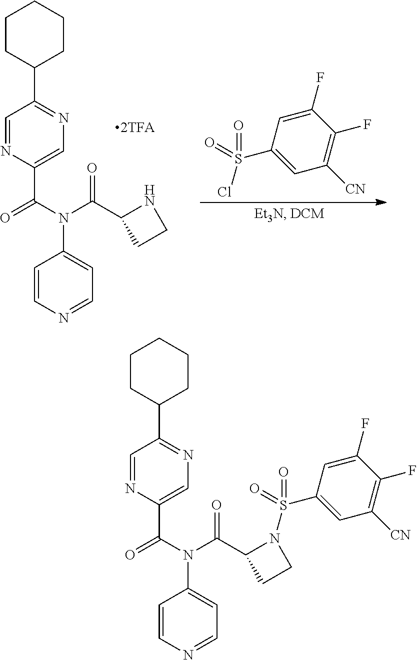 [SMILES: O=C(c1cnc(C2CCCCC2)cn1)N(C(=O)[C@H]1CCN1)c1ccncc1 ; N#Cc1cc(S(=O)(=O)Cl)cc(F)c1F ; N#Cc1cc(S(=O)(=O)N2CC[C@@H]2C(=O)N(C(=O)c2cnc(C3CCCCC3)cn2)c2ccncc2)cc(F)c1F]

### (R)-1-((3-cyano-4,5-difluorophenyl)sulfonyl)-N-((5-cyclohexylpyrazin-2-yl)methyl)-N-(pyridin-4-yl)azetidine-2-carboxamide

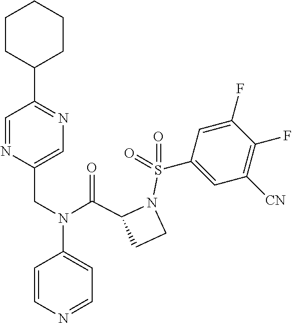 [SMILES: N#Cc1cc(S(=O)(=O)N2CC[C@@H]2C(=O)N(Cc2cnc(C3CCCCC3)cn2)c2ccncc2)cc(F)c1F]

Step 1. To a solution of pyridin-4-amine (300 mg, 3.19 mmol) in ethanol (7.7 mL) was added at 50° C. di-tert-butyl dicarbonate (1.83 mL, 8.0 mmol) under argon. The mixture was stirred at 50° C. for 20 h. The solvent was concentrated to dryness. Trituration with hexane gave tert-butyl pyridin-4-ylcarbamate (562 mg, 91% yield) as a white solid. 1H NMR (300 MHz, Chloroform-d) δ 8.51-8.41 (m, 2H), 7.37-7.24 (m, 2H), 6.73 (bs, 1H), 1.55 (s, 9H).

Step 2. Preparation by a similar procedure to Example 36, step 1, starting from tert-butyl pyridin-4-ylcarbamate (152.5 mg, 0.785 mmol) to obtain tert-butyl ((5-cyclohexylpyrazin-2-yl)methyl)(pyridin-4-yl)carbamate (104.8 mg, 36%). 1H NMR (300 MHz, Chloroform-d) δ 8.56-8.47 (m, 3H), 8.43 (d, J=1.5 Hz, 1H), 7.45-7.35 (m, 2H), 5.03 (s, 2H), 2.84-2.68 (m, 1H), 2.01-1.74 (m, 5H), 1.47 (s, 9H), 1.68-1.23 (m, 5H).

Step 3. Preparation by a similar procedure to Example 7, step 4, starting from tert-butyl pyridin-4-ylcarbamate (104 mg, 0.282 mmol) to obtain crude N-((5-cyclohexylpyrazin-2-yl)methyl)pyridin-4-amine (75.3 mg, as a ca. 1:1 mixture of free base and TFA salt by nmr). 1H NMR (300 MHz, Chloroform-d) δ 8.63-8.16 (m, 4H), 7.13-7.08 (m, 1H), 6.58 (d, J=5.5 Hz, 1H), 5.37 (s, 1H), 4.98 (s, 1H), 4.51 (d, J=5.1 Hz, 1H), 2.78 (t, J=11.8 Hz, 1H), 2.02-1.85 (m, 3H), 1.84-1.73 (m, 1H), 1.74-1.22 (m, 6H).

Step 4. Preparation by a similar procedure to Example 38, step 4, starting from crude N-((5-cyclohexylpyrazin-2-yl)methyl)pyridin-4-amine (73 mg, 0.231 mmol considering 1:1 mixture of free base and TFA salt, see above) to obtain tert-butyl (R)-2-(((5-cyclohexylpyrazin-2-yl)methyl)(pyridin-4-yl)carbamoyl)azetidine-1-carboxylate (24 mg, 25% yield). 1H NMR (300 MHz, Chloroform-d) δ 8.69-8.58 (m, 3H), 8.35 (d, J=1.5 Hz, 1H), 7.46-7.20 (m, 2H), 5.35-4.77 (m, 2H), 4.77-4.58 (m, 1H), 4.08 (q, J=7.8 Hz, 1H), 3.86-3.68 (m, 1H), 2.73 (tt, J=11.9, 3.4 Hz, 1H), 2.27-2.13 (m, 2H), 1.97-1.69 (m, 5H), 1.62-1.22 (m, 14H).

Step 5. Preparation by a similar procedure to Example 38, step 5, starting from tert-butyl (R)-2-(((5-cyclohexylpyrazin-2-yl)methyl)(pyridin-4-yl)carbamoyl)azetidine-1-carboxylate (24.4 mg, 0.054 mmol) to obtain (R)—N-((5-cyclohexylpyrazin-2-yl)methyl)-N-(pyridin-4-yl)azetidine-2-carboxamide TFA salt (33.3 mg) as a yellow oil. 1H NMR (300 MHz, Chloroform-d) δ 8.84-8.73 (m, 2H), 8.66-8.55 (m, 1H), 8.42 (s, 1H), 7.82-7.75 (bs, 1H), 7.73 (dd, J=5.7, 3.3 Hz, 1H), 7.55 (dd, J=5.7, 3.3 Hz, 1H), 5.19 (d, J=16.9 Hz, 1H), 5.03 (d, J=16.9 Hz, 1H), 4.32-3.88 (m, 2H), 2.84-2.43 (m, 3H), 1.99-1.02 (m, 10H).

Step 6. Preparation by a similar procedure to Example 38, step 6, starting from (R)—N-((5-cyclohexylpyrazin-2-yl)methyl)-N-(pyridin-4-yl)azetidine-2-carboxamide TFA salt (33.3 mg, 0.0575 mmol as free base complex with two TFA molecules) to obtain (R)-1-((3-cyano-4,5-difluorophenyl)sulfonyl)-N-((5-cyclohexylpyrazin-2-yl)methyl)-N-(pyri din-4-yl)azetidine-2-carboxamide (3.2 mg, 10% yield). 1H NMR (300 MHz, Chloroform-d) δ 8.76-8.67 (m, 2H), 8.51 (d, J=1.5 Hz, 1H), 8.40 (d, J=1.5 Hz, 1H), 8.22-8.03 (m, 2H), 7.33-7.22 (m, 2H), 5.18-5.06 (m, 2H), 4.90 (d, J=15.7 Hz, 1H), 4.08 (q, J=7.9 Hz, 1H), 3.74-3.60 (m, 1H), 2.76 (t, J=11.4 Hz, 1H), 2.41-2.26 (m, 1H), 2.08-1.83 (m, 4H), 1.82-1.19 (m, 7H). 19F NMR (282 MHz, Chloroform-d) δ−122.65, −129.78.

### Example 42

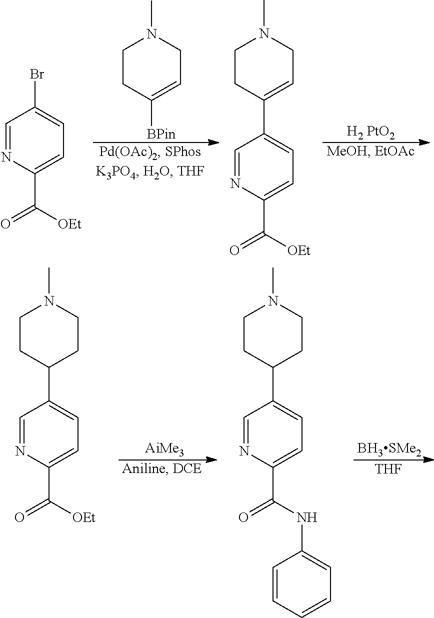

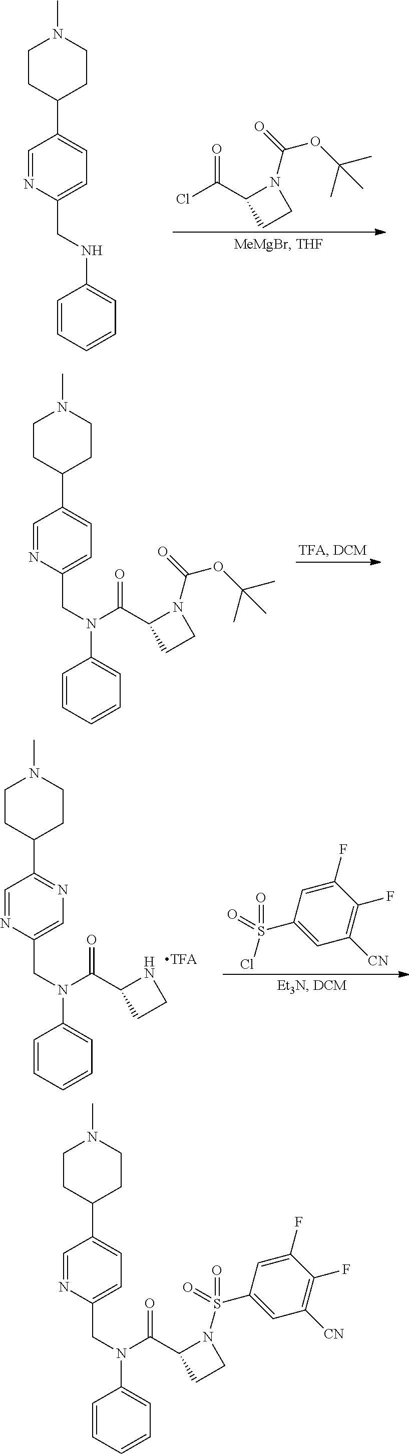

### (R)-1-((3-cyano-4,5-difluorophenyl)sulfonyl)-N-((5-(1-methylpiperidin-4-yl)pyridin-2-yl)methyl)-N-phenylazetidine-2-carboxamide

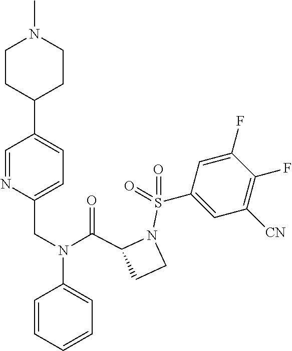 [SMILES: CN1CCC(c2ccc(CN(C(=O)[C@H]3CCN3S(=O)(=O)c3cc(F)c(F)c(C#N)c3)c3ccccc3)nc2)CC1]

Step 1. To ethyl 5-bromopicolinate (1.52 g, 7.02 mmol), under argon, were added palladium acetate (157.7 mg, 0.702 mmol), SPhos (576 mg, 1.404 mmol), 1-methyl-4-(4,4,5,5-tetramethyl-1,3,2-dioxaborolan-2-yl)-1,2,3,6-tetrahydropyridine (2.34 g, 10.53 mmol), potassium phosphate (2.98 g, 14.05 mmol) and deionized water (0.25 mL). The mixture was thoroughly flushed with argon. Dry THF (51 mL) was then added through a syringe. The mixture was stirred at 40° C. (oil bath temperature) for 20 hours. Water was added, and the mixture was extracted with ethyl acetate (2×). The extract was washed with brine, dried (Na2SO4), and concentrated. Purification by flash column chromatography (9:1 to 85:15 DCM/MeOH) gave ethyl 1′-methyl-1′,2′,3′,6′-tetrahydro-[3,4′-bipyridine]-6-carboxylate (420 mg, 26% yield). 1H NMR (300 MHz, Chloroform-d) δ 8.80 (dd, J=2.3, 0.8 Hz, 1H), 8.11 (dd, J=8.2, 0.8 Hz, 1H), 7.81 (dd, J=8.2, 2.3 Hz, 1H), 6.32-6.24 (m, 1H), 4.02 (s, 3H), 3.28-3.19 (m, 2H), 2.82-2.75 (m, 2H), 2.69-2.61 (m, 2H), 2.47 (s, 3H).

Step 2. To a solution of ethyl l′-methyl-1′,2′,3′,6′-tetrahydro-[3,4′-bipyridine]-6-carboxylate (418 mg, 1.8 mmol) in ethyl acetate (3.5 mL) and methanol (3.5 mL) was added platinum oxide (42 mg). A balloon filled with hydrogen was set up, and the mixture was stirred for 16 hours. After filtration and evaporation, the crude was purified by flash column chromatography (DCM/MeOH 85:15 with 5% Et3N) to obtain ethyl 5-(1-methylpiperidin-4-yl)picolinate (324 mg, 77% yield) as a light brown solid. 1H NMR (300 MHz, Chloroform-d) δ 8.64 (d, J=2.2 Hz, 1H), 8.11 (d, J=8.0, 1H), 7.72 (dd, J=8.0, 2.2 Hz, 1H), 4.02 (s, 3H), 3.13-3.02 (m, 2H), 2.73-2.58 (m, 1H), 2.41 (s, 3H), 2.24-2.10 (m, 2H), 1.96-1.86 (m, 4H).

Step 3. Preparation by a similar procedure to Example 40, step 3, starting from ethyl 5-(1-methylpiperidin-4-yl)picolinate (321 mg, 1.37 mmol) to obtain 5-(1-methylpiperidin-4-yl)-N-phenylpicolinamide (187 mg, 46% yield) as a light yellow solid. 1H NMR (300 MHz, Chloroform-d) δ 9.98 (bs, 1H), 8.52 (d, J=2.3 Hz, 1H), 8.26 (d, J=8.1 Hz, 1H), 7.85-7.74 (m, 3H), 7.45-7.36 (m, 2H), 7.20-7.12 (m, 1H), 3.30-3.18 (m, 2H), 2.80-2.66 (m, 1H), 2.53 (s, 3H), 2.44-2.30 (m, 2H), 2.24-2.05 (m, 2H), 2.02-1.89 (m, 2H).

Step 4. Preparation by a similar procedure to Example 39, step 2, starting from 5-(1-methylpiperidin-4-yl)-N-phenylpicolinamide (186 mg, 0.63 mmol) to obtain N-((5-(1-methylpiperidin-4-yl)pyridin-2-yl)methyl)aniline (31 mg, 18% yield). 1H NMR (300 MHz, Chloroform-d) δ 8.48 (d, J=2.3 Hz, 1H), 7.54 (dd, J=8.1, 2.4 Hz, 1H), 7.47-7.26 (m, 1H), 7.24-7.13 (m, 2H), 6.79-6.64 (m, 3H), 4.45 (s, 2H), 3.17-3.05 (m, 2H), 2.65-2.51 (m, 1H), 2.43 (s, 3H), 2.29-2.14 (m, 2H), 2.01-1.81 (m, 4H).

Step 5. Preparation by a similar procedure to Example 38, step 4, starting from N-((5-(1-methylpiperidin-4-yl)pyridin-2-yl)methyl)aniline (67 mg, 0.24 mmol) to obtain tert-butyl (R)-2-(((5-(1-methylpiperidin-4-yl)pyridin-2-yl)methyl)(phenyl)carbamoyl)azetidine-1-carboxylate (50 mg, 45% yield). 1H NMR (300 MHz, Chloroform-d) δ 8.40-8.33 (m, 1H), 7.59 (dd, J=8.1, 2.4 Hz, 1H), 7.50-7.43 (m, 1H), 7.40-7.30 (m, 3H), 7.24-7.12 (m, 2H), 5.04 (s, 2H), 4.64-4.55 (m, 1H), 3.89-3.68 (m, 2H), 3.36-3.23 (m, 4H), 3.06-2.96 (m, 1H), 2.57 (s, 3H), 2.76-1.82 (m, 6H), 1.44 (s, 9H). LRMS (ESI) m/z 465.3 [M+H]+

Step 6. Preparation by a similar procedure to Example 38, step 5, starting from tert-butyl (R)-2-(((5-(1-methylpiperidin-4-yl)pyridin-2-yl)methyl)(phenyl)carbamoyl)azetidine-1-carboxylate (31.1 mg, 0.067 mmol) to obtain, after purification by preparative TLC (DCM/MeOH 85:15), (R)—N-((5-(1-methylpiperidin-4-yl)pyridin-2-yl)methyl)-N-phenylazetidine-2-carboxamide TFA salt (68 mg) as an oil. 1H NMR (300 MHz, Chloroform-d) δ 12.20 (bs, 1H), 8.46 (s, 1H), 7.69-7.56 (m, 1H), 7.45-7.31 (m, 2H), 7.28-7.14 (m, 4H), 5.75-5.67 (m, 1H), 5.12-4.93 (m, 2H), 4.19-4.05 (m, 1H), 4.00-3.78 (m, 2H), 3.77-3.63 (m, 1H), 3.49-3.23 (m, 2H), 2.87 (s, 3H), 3.08-2.74 (m, 2H), 2.48-1.80 (m, 5H).

Step 7. Preparation by a similar procedure to Example 38, step 6, starting from (R)—N-((5-(1-methylpiperidin-4-yl)pyridin-2-yl)methyl)-N-phenylazetidine-2-carboxamide TFA salt (68 mg of TFA salt) to obtain after flash chromatography column purification (9:1 DCM/MeOH) (R)-1-((3-cyano-4,5-difluorophenyl)sulfonyl)-N-((5-(1-methylpiperidin-4-yl)pyridin-2-yl)methyl)-N-phenylazetidine-2-carboxamide (24 mg, from which a sample was further purified by reverse phase semi-prep C18 column (CH3CN/water gradient with 0.1% formic acid)). 1H NMR (300 MHz, Chloroform-d) δ 8.47-8.40 (m, 1H), 8.23-8.13 (m, 1H), 8.08-7.91 (m, 2H), 7.68-7.61 (m, 1H), 7.46-7.36 (m, 2H), 7.35-7.16 (m, 3H), 5.47-5.40 (m, 1H), 5.06-4.94 (m, 2H), 3.91-3.79 (m, 2H), 3.70-3.58 (m, 3H), 3.35-3.24 (m, 1H), 2.84 (s, 3H), 2.76-2.69 (m, 1H), 2.26-1.82 (m, 6H). LRMS (ESI) m/z 566.2 [M+H]+; HRMS (ESI) m/z 565.1953.

### Example 43

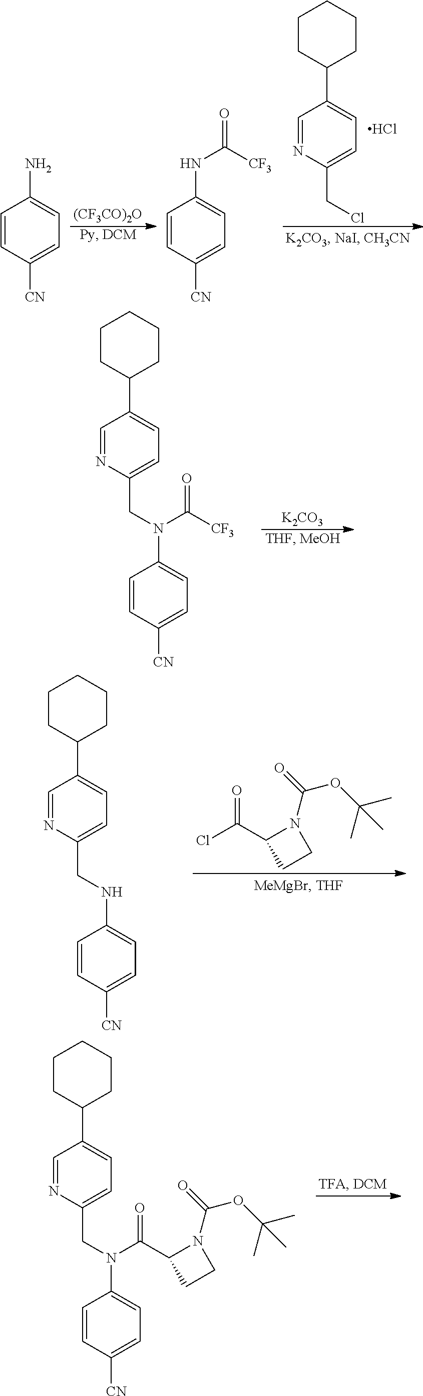

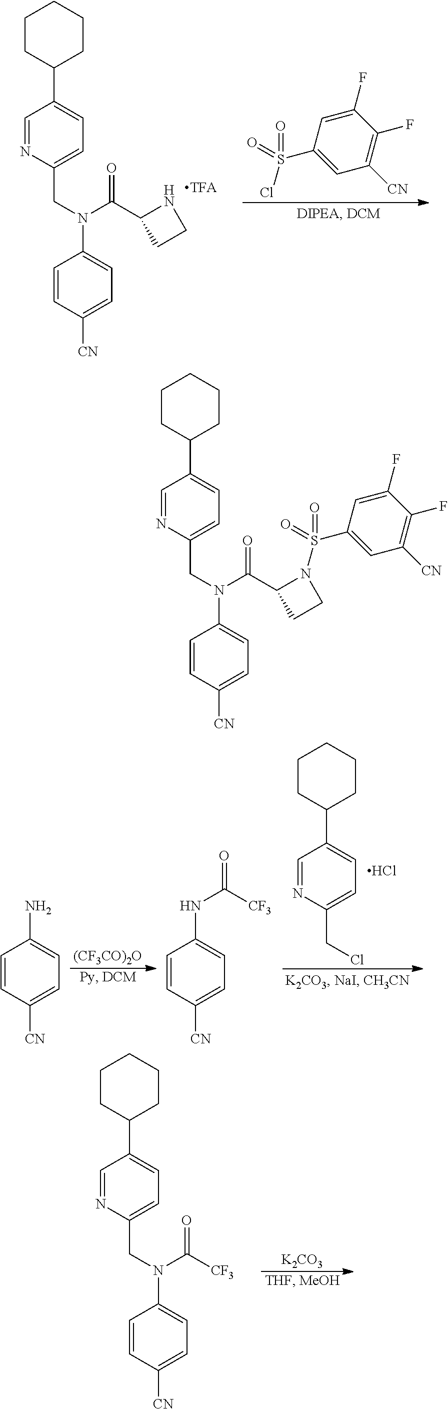

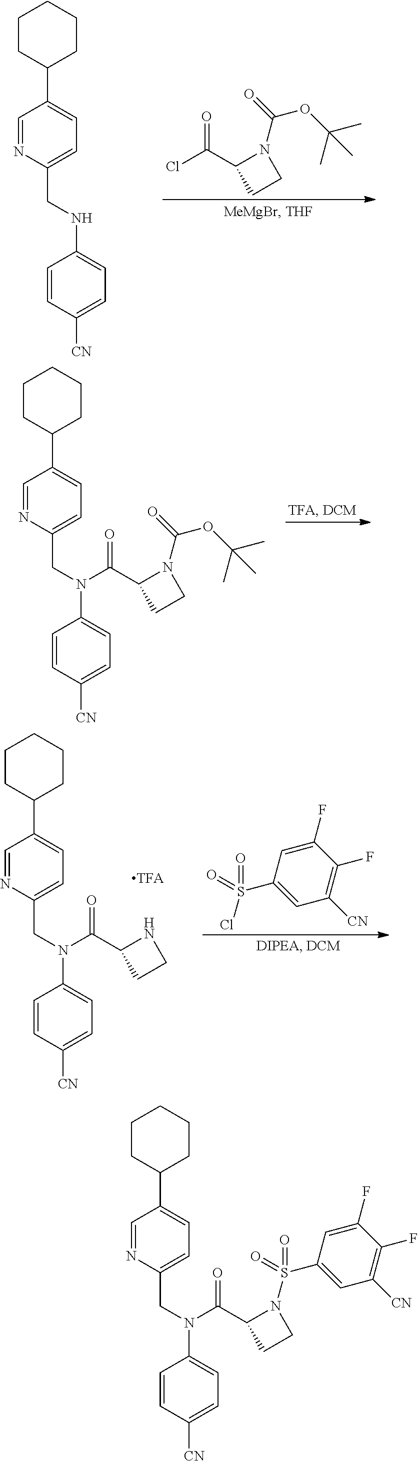

### (R)-1-((3-cyano-4,5-difluorophenyl)sulfonyl)-N-(4-cyanophenyl)-N-((5-cyclohexylpyridin-2-yl)methyl)azetidine-2-carboxamide

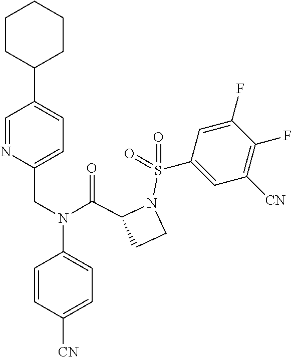 [SMILES: N#Cc1ccc(N(Cc2ccc(C3CCCCC3)cn2)C(=O)[C@H]2CCN2S(=O)(=O)c2cc(F)c(F)c(C#N)c2)cc1]

Step 1. Preparation by a similar procedure to Example 5, step 1, starting from 4-aminobenzonitrile (1.04 g, 8.8 mmol) to obtain N-(4-cyanophenyl)-2,2,2-trifluoroacetamide (873 mg, 46% yield) as a cream solid. 1H NMR (300 MHz, Chloroform-d) δ 8.29-8.16 (bs, 1H), 7.82-7.67 (m, 4H).

Step 2. To 2-(chloromethyl)-5-cyclohexylpyridine hydrochloride (434 mg, 1.76 mmol), N-(4-cyanophenyl)-2,2,2-trifluoroacetamide (299 mg, 1.40 mmol), sodium iodide (12.6 mg, 0.084 mmol) was added under argon acetonitrile (18 mL), followed by sodium carbonate (675.2 mg, 4.90 mmol). The mixture was heated at 65° C. for 18 hours. After cooling, aqueous ammonium chloride was added, and mixture was extracted with ethyl acetate (2×). The extract was washed with brine, dried (Na2SO4), and concentrated. Purification by flash column chromatography (6:4 hexane/ethyl acetate) gave N-(4-cyanophenyl)-N-((5-cyclohexylpyridin-2-yl)methyl)-2,2,2-trifluoroacetamide (458.2 mg, slightly contaminated with starting material). 1H NMR (300 MHz, Chloroform-d) δ 8.40 (d, J=2.2 Hz, 1H), 7.73-7.66 (m, 2H), 7.57 (dd, J=8.1, 2.2 Hz, 1H), 7.47-7.37 (m, 2H), 7.35-7.24 (m, 1H), 5.06 (s, 2H), 2.62-2.48 (m, 1H), 1.94-1.72 (m, 5H), 1.52-1.19 (m, 5H).

Step 3. Preparation by a similar procedure to Example 6, step 2, starting from N-(4-cyanophenyl)-N-((5-cyclohexylpyridin-2-yl)methyl)-2,2,2-trifluoroacetamide (458 mg, 1.18 mmol) to obtain 4-(((5-cyclohexylpyridin-2-yl)methyl)amino)benzonitrile (244 0.2 mg, 71% yield). 1H NMR (500 MHz, Chloroform-d) δ 8.47 (d, J=2.2 Hz, 1H), 7.74 (d, J=8.0 Hz, 1H), 7.47-7.38 (m, 3H), 6.71-6.62 (m, 2H), 5.87 (bs, 1H), 4.59 (s, 2H), 2.65-2.54 (m, 1H), 1.94-1.75 (m, 5H), 1.49-1.36 (m, 4H), 1.32-1.22 (m, 1H).

Step 4. Preparation by a similar procedure to Example 38, step 4, starting from 4-(((5-cyclohexylpyridin-2-yl)methyl)amino)benzonitrile (46.6 mg, 0.16 mmol) to obtain tert-butyl (R)-2-((4-cyanophenyl)((5-cyclohexylpyridin-2-yl)methyl)carbamoyl)azetidine-1-carboxylate (55.6 mg, 73% yield). 1H NMR (500 MHz, Chloroform-d) δ 8.35 (s, 1H), 7.67 (d, J=8.0 Hz, 2H), 7.63-7.33 (m, 4H), 5.23-4.94 (m, 2H), 4.67-4.53 (m, 1H), 4.16-4.06 (m, 1H), 3.79 (q, J=7.7 Hz, 1H), 2.58-2.48 (m, 1H), 2.25-2.13 (m, 2H), 1.92-1.73 (m, 5H), 1.51-1.20 (m, 14H).

Step 5. Preparation by a similar procedure to Example 38, step 5, starting from tert-butyl (R)-2-((4-cyanophenyl)((5-cyclohexylpyridin-2-yl)methyl)carbamoyl)azetidine-1-carboxylate (46.6 mg, 0.098 mmol) to obtain crude (R)—N-(4-cyanophenyl)-N-((5-cyclohexylpyridin-2-yl)methyl)azetidine-2-carboxamide (72.1 mg, complexed with about 3 TFA molecules). 1H NMR (500 MHz, Chloroform-d) δ 8.44-8.30 (m, 1H), 7.72-7.60 (m, 2H), 7.53-7.48 (m, 1H), 7.42-7.35 (m, 2H), 7.24-7.18 (m, 1H), 5.1-4.86 (m, 2H), 4.81-4.32 (m, 1H), 3.94-3.74 (m, 2H), 2.62-2.42 (m, 2H), 2.40-1.73 (m, 5H), 1.49-1.16 (m, 5H).

Step 6. Preparation by a similar procedure to Example 38, step 6, starting from crude (R)—N-(4-cyanophenyl)-N-((5-cyclohexylpyridin-2-yl)methyl)azetidine-2-carboxamide (72.1 mg, 0.10 mmol, if complexed with 3 TFA molecules) to obtain (R)-1-((3-cyano-4,5-difluorophenyl)sulfonyl)-N-(4-cyanophenyl)-N-((5-cyclohexylpyridin-2-yl)methyl)azetidine-2-carboxamide (55 mg, 95% yield). 1H NMR (500 MHz, Chloroform-d) δ 8.39 (s, 1H), 8.15 (ddd, J=8.0, 6.9, 2.2 Hz, 1H), 8.08 (m, 1H), 7.72 (d, J=8.1 Hz, 2H), 7.59 (d, J=7.7 Hz, 1H), 7.43 (d, J=8.1 Hz, 2H), 7.34-7.28 (m, 1H), 5.10-4.93 (m, 3H), 4.08-4.00 (m, 1H), 3.70-3.63 (m, 1H), 2.60-2.50 (s, 1H), 2.43-2.32 (m, 1H), 2.03-1.71 (m, 6H), 1.49-1.33 (m, 4H), 1.32-1.21 (m, 1H). 19F NMR (282 MHz, Chloroform-d) δ−122.58, −129.83.

### Example 44

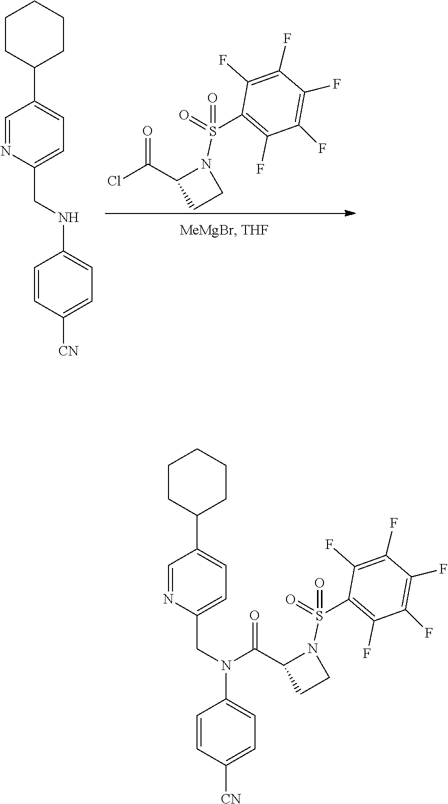 [SMILES: N#Cc1ccc(NCc2ccc(C3CCCCC3)cn2)cc1 ; O=C(Cl)[C@H]1CCN1S(=O)(=O)c1c(F)c(F)c(F)c(F)c1F ; N#Cc1ccc(N(Cc2ccc(C3CCCCC3)cn2)C(=O)[C@H]2CCN2S(=O)(=O)c2c(F)c(F)c(F)c(F)c2F)cc1]

### (R)—N-(4-cyanophenyl)-N-((5-cyclohexylpyridin-2-yl)methyl)-1-((perfluorophenyl)sulfonyl)azetidine-2-carboxamide

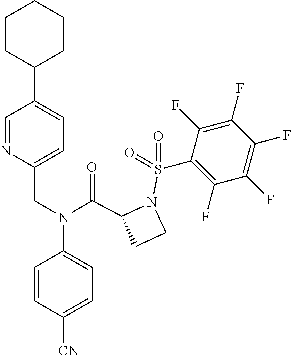 [SMILES: N#Cc1ccc(N(Cc2ccc(C3CCCCC3)cn2)C(=O)[C@H]2CCN2S(=O)(=O)c2c(F)c(F)c(F)c(F)c2F)cc1]

Step 1. Preparation by a similar procedure to Example 22, step 1, starting from 4-(((5-cyclohexylpyridin-2-yl)methyl)amino)benzonitrile (360 mg, 1.24 mmol) to obtain (R)—N-(4-cyanophenyl)-N-((5-cyclohexylpyridin-2-yl)methyl)-1-((perfluorophenyl)sulfonyl) azetidine-2-carboxamide (105.8 mg, 15% yield) as a white foam. 1H NMR (300 MHz, Chloroform-d) δ 8.34 (s, 1H), 7.70 (d, J=8.0 Hz, 2H), 7.56-7.46 (m, 1H), 7.38 (d, J=8.0 Hz, 2H), 7.19 (d, J=8.1 Hz, 1H), 5.07-4.81 (m, 3H), 4.23-4.02 (m, 2H), 2.59-2.43 (m, 1H), 2.41-2.25 (m, 1H), 2.13-1.96 (m, 1H), 1.94-1.73 (m, 5H), 1.50-1.21 (m, 5H). 19F NMR (282 MHz, Chloroform-d) δ−135.88, −146.68, −159.40.

### Example 45

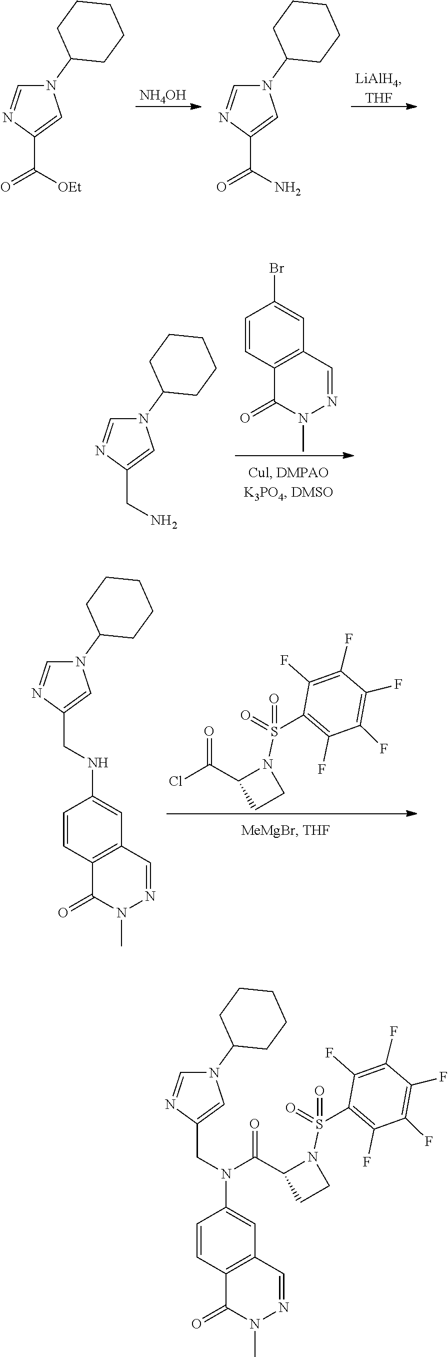

### (R)—N-((1-cyclohexyl-1H-imidazol-4-yl)methyl)-N-(2-methyl-1-oxo-1,2-dihydrophthalazin-6-yl)-1-((perfluorophenyl)sulfonyl)azetidine-2-carboxamide

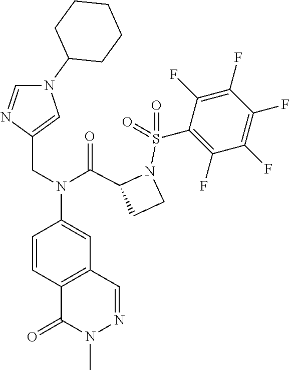 [SMILES: Cn1ncc2cc(N(Cc3cn(C4CCCCC4)cn3)C(=O)[C@H]3CCN3S(=O)(=O)c3c(F)c(F)c(F)c(F)c3F)ccc2c1=O]

Step 1. In a sealed tube, a mixture of ethyl 1-cyclohexyl-1H-imidazole-4-carboxylate (1.12 g, 5.03 mmol, see Example 140 for preparation) and concentrated ammonium hydroxide (38 mL) was heated at 90° C. for 22 hours under argon. After cooling, the mixture was diluted with brine and extracted with dichloromethane (3×). The extract was dried (Na2SO4) and concentrated to dryness to obtain crude 1-cyclohexyl-1H-imidazole-4-carboxamide (720 mg, 74% yield). 1H NMR (500 MHz, Chloroform-d) δ 7.70 (s, 1H), 7.61 (s, 1H), 7.19 (bs, 1H), 5.48 (bs, 1H), 4.03-3.93 (m, 1H), 2.21-1.86 (m, 4H), 1.81-1.74 (m, 1H), 1.64 (m, 2H), 1.51-1.34 (m, 2H), 1.33-1.21 (m, 1H).

Step 2. To a solution of crude 1-cyclohexyl-1H-imidazole-4-carboxamide (720 mg, 3.73 mmol) in THF (22 mL) was added at 0° C. 2M in THF LiAlH4 (3.73 mL, 7.46 mmol) under argon. The mixture was heated at 66° C. for 17 hours. The mixture was cooled to 0° C. and water, followed by 1M KOH were added. The mixture was extracted with DCM (2×). The extract was dried (NaSO4) and concentrated. Purification by flash column chromatography (85:15:1 DCM/MeOH/NH4OH) gave (1-cyclohexyl-1H-imidazol-4-yl)methanamine (357 mg, 54% yield) as a pale yellow oil. 1H NMR (500 MHz, Chloroform-d) δ 7.49 (s, 1H), 6.85 (s, 1H), 3.86 (tt, J=12.4, 3.6 Hz, 1H), 3.81 (s, 2H), 2.10 (dd, J=13.5, 3.6 Hz, 2H), 1.90 (dt, J=13.5, 3.6 Hz, 2H), 1.79-1.72 (m, 1H), 1.62 (qd, J=12.4, 3.6 Hz, 2H), 1.41 (qt, J=13.5, 3.6 Hz, 2H), 1.24 (qt, J=13.5, 3.6 Hz, 1H).

Step 3. See Yu Zhang, Xinye Yang, Qizheng Yao, and Dawei Ma Org. Lett. 2012, 14, 12, 3056-3059. Under argon, to (1-cyclohexyl-1H-imidazol-4-yl)methanamine (285 mg, 1.59 mmol) were added CuI (20.1 mg, 0.106 mmol), [2,6-dimethylphenyl)carbamoyl]formic acid (DMPAO, 40.4 mg, 0.212 mmol), 6-bromo-2-methylphthalazin-1(2H)-one (254 mg, 1.06 mmol) and K3PO4 (450 mg, 2.12 mmol). The mixture was thoroughly flushed with argon, and then dry DMSO (4 mL) was added. The mixture was heated at 92° C. (oil bath temperature) for 24 hours. After cooling, water was added, and the mixture was extracted with ethyl acetate (2×). The extract was washed with water, brine, dried (Na2SO4) and concentrated. Purification by flash column chromatography (85:15:1 DCM/MeOH/NH4OH) gave 6-(((1-cyclohexyl-1H-imidazol-4-yl)methyl)amino)-2-methylphthalazin-1(2H)-one (297.4 mg, 83% yield) as a yellow solid. 1H NMR (300 MHz, Chloroform-d) δ 8.19 (d, J=8.7 Hz, 1H), 7.98 (d, J=0.9 Hz, 1H), 7.55 (s, 1H), 7.03 (dd, J=8.7, 2.4, 0.9 Hz, 1H), 6.92 (s, 1H), 6.68 (d, J=2.4 Hz, 1H), 5.08 (bs, 1H), 4.36 (d, J=4.1 Hz, 2H), 3.96-3.83 (m, 1H), 3.81 (s, 3H), 2.21-1.71 (m, 5H), 1.70-1.16 (m, 5H).

Step 4. Preparation by a similar procedure to Example 22, step 1, starting from 6-(((1-cyclohexyl-1H-imidazol-4-yl)methyl)amino)-2-methylphthalazin-1(2H)-one (101.7 mg, 0.301 mmol) to obtain (R)—N-((1-cyclohexyl-1H-imidazol-4-yl)methyl)-N-(2-methyl-1-oxo-1,2-dihydrophthalazin-6-yl)-1-((perfluorophenyl)sulfonyl)azetidine-2-carboxamide (56 mg, 29% yield). 1H NMR (300 MHz, Chloroform-d) δ 8.42 (d, J=8.4 Hz, 1H), 8.12 (s, 1H), 7.59-7.43 (m, 3H), 6.88 (s, 1H), 4.96-4.74 (m, 3H), 4.16-3.95 (m, 2H), 3.87 (s, 3H), 3.93-3.76 (m, 1H), 2.36-2.21 (m, 1H), 2.14-1.71 (m, 6H), 1.70-1.12 (m, 5H). 19F NMR (282 MHz, Chloroform-d) δ−135.88, −146.83, −159.62.

### Example 46

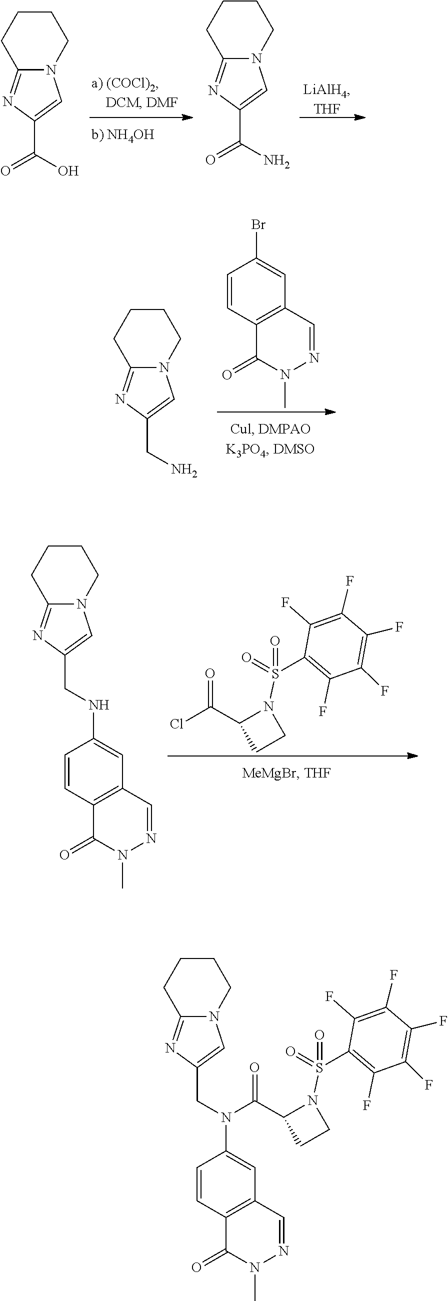

### (R)—N-(2-methyl-1-oxo-1,2-dihydrophthalazin-6-yl)-1-((perfluorophenyl)sulfonyl)-N-((5,6,7,8-tetrahydroimidazo[1,2-a]pyridin-2-yl)methyl)azetidine-2-carboxamide

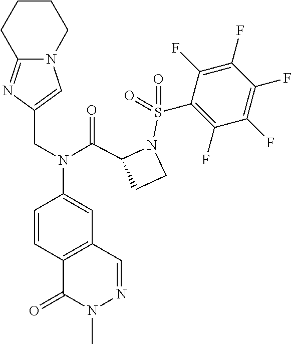 [SMILES: Cn1ncc2cc(N(Cc3cn4c(n3)CCCC4)C(=O)[C@H]3CCN3S(=O)(=O)c3c(F)c(F)c(F)c(F)c3F)ccc2c1=O]

Step 1. To a suspension of 5,6,7,8-tetrahydroimidazo[1,2-a]pyridine-2-carboxylic acid (505 mg, 3.04 mmol) in dichloromethane (26 mL) was added under argon oxalyl chloride (0.46 mL, 5.36 mmol), followed by DMF (one drop). The mixture was stirred for 23 h, and then concentrated to dryness. Dry acetonitrile (10 mL) was added under argon, and the mixture was cooled to 0° C. Concentrated ammonium hydroxide (10 mL) was added. The mixture was allowed to reach room temperature and stirred for 2.5 hours. The mixture was concentrated to small volume (ca. 1-1.5 mL). During concentration, acetonitrile was added (×3) to azeotrope water. To the resulting wet solid was added dichloromethane/methanol 95:5 (30 mL). After stirring for ca. 40 minutes, the phases were separated. The organic phase was dried (Na2SO4), and concentrated to dryness to obtain 5,6,7,8-tetrahydroimidazo[1,2-a]pyridine-2-carboxamide (318 mg, 63% yield) as a white solid. 1H NMR (300 MHz, DMSO-d6) δ 7.45 (s, 1H), 7.21 (bs, 1H), 6.98 (bs, 1H), 3.96 (t, J=5.6 Hz, 2H), 2.72 (t, J=6.0 Hz, 2H), 1.93-1.79 (m, 4H).

Step 2. Preparation by a similar procedure to Example 45, step 2, starting from 5,6,7,8-tetrahydroimidazo[1,2-a]pyridine-2-carboxamide (312 mg, 1.89 mmol) to obtain (5,6,7,8-tetrahydroimidazo[1,2-a]pyridin-2-yl)methanamine (168 mg, 59% yield). 1H NMR (300 MHz, Chloroform-d) δ 6.65 (s, 1H), 3.97-3.86 (m, 2H), 3.76 (s, 2H), 2.85 (t, J=6.2 Hz, 2H), 2.10-1.85 (m, 4H).

Step 3. Preparation by a similar procedure to Example 45, step 3, starting from (5,6,7,8-tetrahydroimidazo[1,2-a]pyridin-2-yl)methanamine (104.2 mg, 0.435 mmol) to obtain 2-methyl-6-(((5,6,7,8-tetrahydroimidazo[1,2-a]pyridin-2-yl)methyl)amino)phthalazin-1(2H)-one (41 mg, 30% yield) as a yellow oil. 1H NMR (300 MHz, Chloroform-d) δ 8.70 (bs, 1H), 8.13 (d, J=8.8 Hz, 1H), 7.95 (s, 1H), 7.04 (dd, J=8.8, 2.3 Hz, 1H), 6.74 (s, 1H), 6.64 (d, J=2.3 Hz, 1H), 4.32 (s, 2H), 3.96-3.84 (m, 2H), 3.78 (s, 3H), 2.86 (t, J=6.2 Hz, 2H), 2.02-1.84 (m, 4H).

Step 4. Preparation by a similar procedure to Example 22, step 1, starting from 2-methyl-6-(((5,6,7,8-tetrahydroimidazo[1,2-a]pyridin-2-yl)methyl)amino)phthalazin-1(2H)-one (39.5, 0.128 mmol) to obtain (R)—N-(2-methyl-1-oxo-1,2-dihydrophthalazin-6-yl)-1-((perfluorophenyl)sulfonyl)-N-((5,6,7,8-tetrahydroimidazo[1,2-a]pyridin-2-yl)methyl)azetidine-2-carboxamide (27.6 mg, 35% yield) as a white foam. 1H NMR (300 MHz, Chloroform-d) δ 8.44 (d, J=8.4 Hz, 1H), 8.13 (s, 1H), 7.63 (d, J=2.3 Hz, 1H), 7.55 (dd, J=8.4, 2.3 Hz, 1H), 6.68 (s, 1H), 5.06-4.65 (m, 3H), 4.23-4.01 (m, 2H), 3.88 (s, 3H), 3.96-3.84 (m, 2H), 2.85-2.74 (m, 2H), 2.37-2.21 (m, 1H), 2.04-1.80 (m, 5H). 19F NMR (282 MHz, Chloroform-d) δ−135.88, −146.83, −159.62.

### Example 47

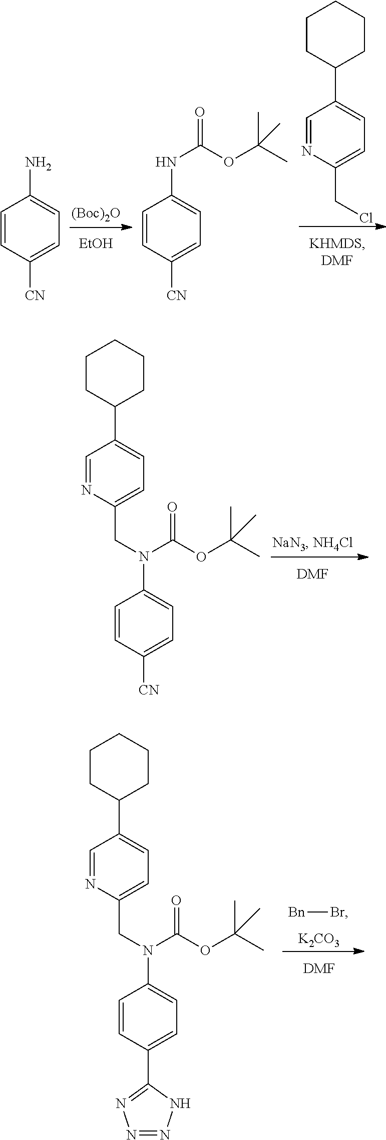

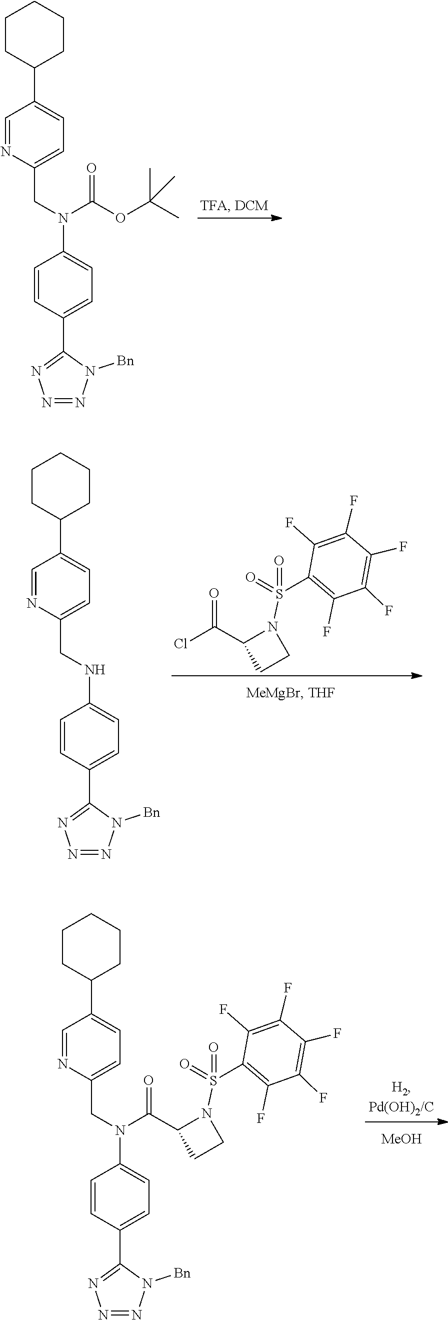

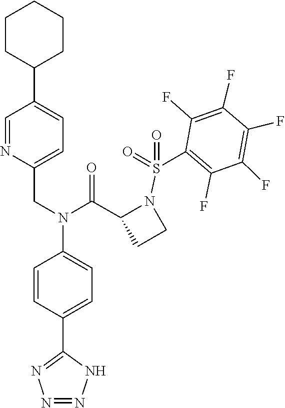 [SMILES: O=C([C@H]1CCN1S(=O)(=O)c1c(F)c(F)c(F)c(F)c1F)N(Cc1ccc(C2CCCCC2)cn1)c1ccc(-c2nnn[nH]2)cc1]

### (R)—N-(4-(1H-tetrazol-5-yl)phenyl)-N-((5-cyclohexylpyridin-2-yl)methyl)-1-((perfluorophenyl)sulfonyl)azetidine-2-carboxamide

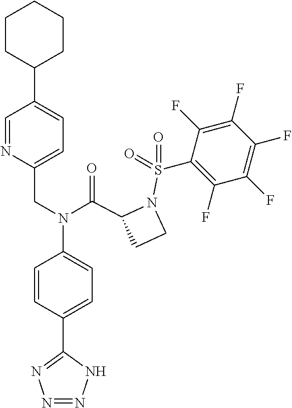 [SMILES: O=C([C@H]1CCN1S(=O)(=O)c1c(F)c(F)c(F)c(F)c1F)N(Cc1ccc(C2CCCCC2)cn1)c1ccc(-c2nnn[nH]2)cc1]

Step 1. To a solution of 4-aminobenzonitrile (1.5 g, 12.65 mmol) in ethanol (24 mL) was added di-tert-butyl dicarbonate (8.7 mL, 37.96 mmol) under argon. The mixture was heated at 72° C. for 42 h, and then concentrated. Purification by flash column chromatography (85:15 hexane/ethyl acetate) gave tert-butyl (4-cyanophenyl)carbamate (1.87 g, 68% yield). 1H NMR (300 MHz, Chloroform-d) δ 7.65-7.54 (m, 2H), 7.55-7.45 (m, 2H), 6.72 (s, 1H), 1.54 (s, 9H).

Step 2. Preparation by a similar procedure to Example 7, step 3, starting from tert-butyl (4-cyanophenyl)carbamate (903 mg, 4.14 mmol) to obtain tert-butyl (4-cyanophenyl)((5-cyclohexylpyridin-2-yl)methyl)carbamate (1.31 g, 81% yield) as a pale yellow solid. 1H NMR (300 MHz, Chloroform-d) δ 8.42 (d, J=2.3 Hz, 1H), 7.65-7.44 (m, 5H), 7.22-7.15 (m, 1H), 4.97 (s, 2H), 2.60-2.48 (m, 1H), 1.92-1.74 (m, 5H), 1.43 (s, 9H), 1.63-1.34 (m, 5H).

Step 3. To a solution of tert-butyl (4-cyanophenyl)((5-cyclohexylpyridin-2-yl)methyl)carbamate (395 mg, 1.0 mmol) in DMF (6 mL) were added sodium azide (165 mg, 2.5 mmol) and ammonium chloride (136 mg, 2.5 mmol) under argon. The mixture was heated at 140° C. with vigorous stirring for 18 hours. After cooling pH 2 buffer was added, and the mixture was extracted with ethyl acetate (2×). The extract was washed with water (2×), brine, dried (Na2SO4) and concentrated. Purification by flash column chromatography (1:1 hexane/EtOAc with 1% acetic acid) gave tert-butyl (4-(1H-tetrazol-5-yl)phenyl)((5-cyclohexylpyridin-2-yl)methyl)carbamate (294 mg, 67% yield). 1H NMR (300 MHz, Chloroform-d) δ 8.41 (d, J=2.2 Hz, 1H), 7.76-7.63 (m, 3H), 7.50 (d, J=8.1 Hz, 1H), 7.34-7.12 (m, 3H), 5.03 (s, 2H), 2.70-2.47 (m, 1H), 1.95-1.71 (m, 5H), 1.45 (s, 9H), 1.53-1.20 (m, 5H).

Step 4. To a solution of tert-butyl (4-(1H-tetrazol-5-yl)phenyl)((5-cyclohexylpyridin-2-yl)methyl)carbamate (288 mg, 0.66 mmol) in DMF (3.3 mL) was added potassium carbonate (97.7 mg, 0.729 mmol) under argon. After 10 minutes, benzyl bromide (0.077 mL, 0.63 mmol) was added. The mixture was stirred for 3 hours, then poured onto cold water. The mixture was extracted with ethyl acetate (2×). The extract was washed with water, brine, dried (Na2SO4) and concentrated. Purification by flash column chromatography (7:3 hexane/EtOAc) gave tert-butyl (4-(1-benzyl-1H-tetrazol-5-yl)phenyl)((5-cyclohexylpyridin-2-yl)methyl)carbamate (246 mg, 71% yield). 1H NMR (300 MHz, Chloroform-d) δ 8.40 (d, J=2.3 Hz, 1H), 8.12-8.00 (m, 2H), 7.52 (dd, J=8.1, 2.3 Hz, 1H), 7.47-7.33 (m, 6H), 7.34-7.21 (m, 2H), 5.81 (s, 2H), 5.00 (s, 2H), 2.60-2.46 (m, 1H), 1.92-1.71 (m, 5H), 1.43 (s, 9H), 1.50-1.19 (m, 5H).

Step 5. Preparation by a similar procedure to Example 7, step 4, starting from tert-butyl (4-(1-benzyl-1H-tetrazol-5-yl)phenyl)((5-cyclohexylpyridin-2-yl)methyl)carbamate (238 mg, 0.45 mmol) to obtain crude 4-(1-benzyl-1H-tetrazol-5-yl)-N-((5-cyclohexylpyridin-2-yl)methyl)aniline (175 mg, 91% yield). 1H NMR (300 MHz, Chloroform-d) δ 8.45 (d, J=2.3 Hz, 1H), 8.02-7.90 (m, 2H), 7.51 (dd, J=8.0, 2.3 Hz, 1H), 7.46-7.32 (m, 5H), 7.26 (d, J=8.0 Hz, 1H), 6.80-6.70 (m, 2H), 5.78 (s, 2H), 5.12 (bs, 1H), 4.48 (s, 2H), 2.62-2.48 (m, 1H), 1.97-1.73 (m, 5H), 1.53-1.20 (m, 5H).

Step 6. Preparation by a similar procedure to Example 22, step 1, starting from crude 4-(1-benzyl-1H-tetrazol-5-yl)-N-((5-cyclohexylpyridin-2-yl)methyl)aniline (172 mg, 0.41 mmol) to obtain (R)—N-(4-(1-benzyl-1H-tetrazol-5-yl)phenyl)-N-((5-cyclohexylpyridin-2-yl)methyl)-1-((perfluorophenyl)sulfonyl)azetidine-2-carboxamide (189 mg, 63% yield). 1H NMR (300 MHz, Chloroform-d) δ 8.33 (d, J=2.3 Hz, 1H), 8.14 (d, J=8.3 Hz, 2H), 7.51 (dd, J=8.0, 2.3 Hz, 1H), 7.56-7.35 (m, 6H), 7.34-7.15 (m, 2H), 5.83 (s, 2H), 5.10-4.84 (m, 3H), 4.19-3.98 (m, 2H), 2.57-2.45 (m, 1H), 2.40-2.27 (m, 1H), 2.03-1.71 (m, 6H), 1.53-1.20 (m, 5H). 19F NMR (282 MHz, Chloroform-d) δ−135.92, −147.00, −159.59.

Step 7. Preparation by a similar procedure to Example 1, step 8, starting from (R)—N-(4-(1-benzyl-1H-tetrazol-5-yl)phenyl)-N-((5-cyclohexylpyridin-2-yl)methyl)-1-((perfluorophenyl)sulfonyl)azetidine-2-carboxamide (171.5 mg, 0.232 mmol) to obtain (R)—N-(4-(1H-tetrazol-5-yl)phenyl)-N-((5-cyclohexylpyridin-2-yl)methyl)-1-((perfluorophenyl)sulfonyl)azetidine-2-carboxamide (46.5 mg, 31% yield). 1H NMR (300 MHz, Chloroform-d) δ 8.41 (s, 1H), 7.94-7.74 (m, 2H), 7.73-7.50 (m, 1H), 7.45-7.35 (m, 2H), 6.96-6.87 (m, 2H), 5.4-5.22 (m, 1H), 5.06-4.86 (m, 2H), 4.16-3.92 (m, 2H), 2.71-2.54 (m, 1H), 2.29-2.15 (m, 1H), 1.99-1.70 (m, 6H), 1.53-1.19 (m, 5H). 19F NMR (282 MHz, Chloroform-d) δ−135.92, −147.00, −159.59. 19F NMR (282 MHz, Chloroform-d) δ−135.66, −146.83, −159.62.

### Example 48

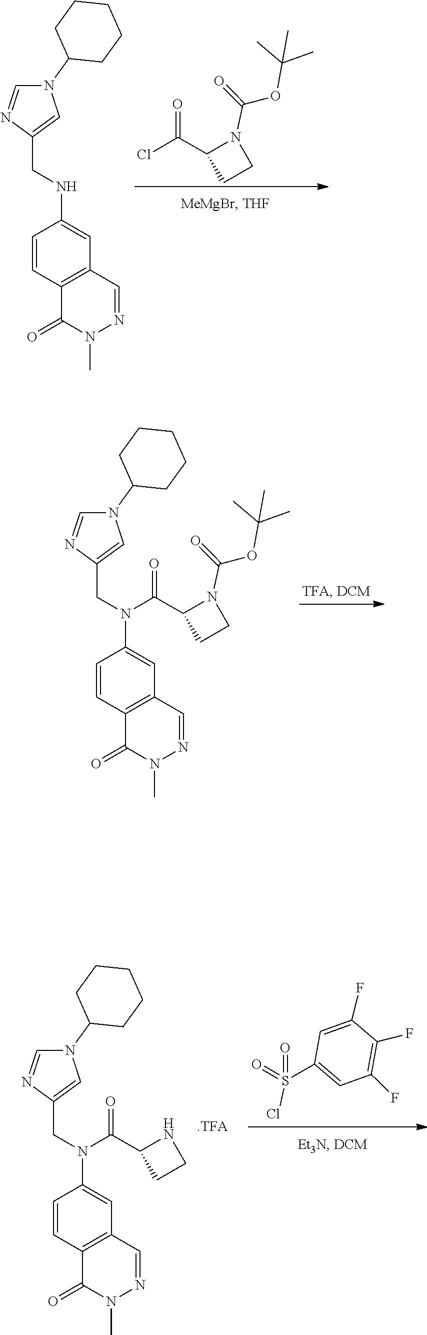 [SMILES: Cn1ncc2cc(NCc3cn(C4CCCCC4)cn3)ccc2c1=O ; CC(C)(C)OC(=O)N1CC[C@@H]1C(=O)Cl ; Cn1ncc2cc(N(Cc3cn(C4CCCCC4)cn3)C(=O)[C@H]3CCN3C(=O)OC(C)(C)C)ccc2c1=O ; Cn1ncc2cc(N(Cc3cn(C4CCCCC4)cn3)C(=O)[C@H]3CCN3)ccc2c1=O ; O=S(=O)(Cl)c1cc(F)c(F)c(F)c1]

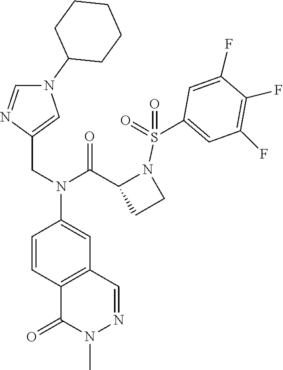 [SMILES: Cn1ncc2cc(N(Cc3cn(C4CCCCC4)cn3)C(=O)[C@H]3CCN3S(=O)(=O)c3cc(F)c(F)c(F)c3)ccc2c1=O]

### (R)-1-((3-cyano-4,5-difluorophenyl)sulfonyl)-N-((1-cyclohexyl-1H-imidazol-4-yl)methyl)-N-(2-methyl-1-oxo-1,2-dihydrophthalazin-6-yl)azetidine-2-carboxamide

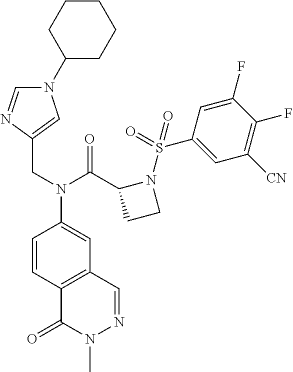 [SMILES: Cn1ncc2cc(N(Cc3cn(C4CCCCC4)cn3)C(=O)[C@H]3CCN3S(=O)(=O)c3cc(F)c(F)c(C#N)c3)ccc2c1=O]

Step 1. Preparation by a similar procedure to Example 38, step 4, starting from 6-(((1-cyclohexyl-1H-imidazol-4-yl)methyl)amino)-2-methylphthalazin-1(2H)-one (183 mg, 0.54 mmol) to obtain tert-butyl (R)-2-(((1-cyclohexyl-1H-imidazol-4-yl)methyl)(2-methyl-1-oxo-1,2-dihydrophthalazin-6-yl) carbamoyl)azetidine-1-carboxylate (235 mg, 83% yield) of a yellow foam. 1H NMR (300 MHz, Chloroform-d) δ 8.42 (d, J=8.6 Hz, 1H), 8.12 (s, 1H), 7.78-7.56 (m, 1H), 7.49 (s, 1H), 7.26-7.08 (m, 2H), 5.06-4.80 (m, 2H), 4.62-4.45 (m, 1H), 4.14-4.05 (m, 1H), 3.87 (s, 3H), 3.91-3.69 (m, 2H), 2.28-2.01 (m, 4H), 1.98-1.83 (m, 2H), 1.82-1.70 (m, 1H), 1.69-1.23 (m, 5H).

Step 2. Preparation by a similar procedure to Example 38, step 5, starting from tert-butyl (R)-2-(((1-cyclohexyl-1H-imidazol-4-yl)methyl)(2-methyl-1-oxo-1,2-dihydrophthalazin-6-yl) carbamoyl)azetidine-1-carboxylate to obtain (R)—N-((1-cyclohexyl-1H-imidazol-4-yl)methyl)-N-(2-methyl-1-oxo-1,2-dihydrophthalazin-6-yl)azetidine-2-carboxamide TFA salt (184.5 mg as TFA salt). 1H NMR (300 MHz, Chloroform-d) δ 8.44 (d, J=8.4 Hz, 1H), 8.13 (s, 1H), 7.63 (s, 1H), 7.56 (s, 1H), 7.44 (d, J=8.4 Hz, 1H), 6.72 (s, 1H), 5.19-5.05 (m, 1H), 4.98 (d, J=14.7 Hz, 1H), 4.80 (d, J=14.7 Hz, 1H), 4.05-3.94 (m, 1H), 3.87 (s, 3H), 3.92-3.73 (m, 2H), 2.73-2.19 (m, 2H), 2.12-1.64 (m, 5H), 1.63-1.11 (m, 5H).

Step 3. Preparation by a similar procedure to Example 38, step 6, starting from (R)—N-((1-cyclohexyl-1H-imidazol-4-yl)methyl)-N-(2-methyl-1-oxo-1,2-dihydrophthalazin-6-yl)azetidine-2-carboxamide TFA salt (184.5 mg, 0.439 mmol) to obtain (R)-1-((3-cyano-4,5-difluorophenyl)sulfonyl)-N-((1-cyclohexyl-1H-imidazol-4-yl)methyl)-N-(2-methyl-1-oxo-1,2-dihydrophthalazin-6-yl)azetidine-2-carboxamide (103 mg, 37% for two steps). 1H NMR (300 MHz, Chloroform-d) δ 8.44 (d, J=8.4 Hz, 1H), 8.26-7.99 (m, 3H), 7.63-7.45 (m, 3H), 6.93 (s, 1H), 5.42-5.01 (m, 1H), 4.94 (d, J=14.8 Hz, 2H), 4.82 (d, J=14.8 Hz, 1H), 3.87 (s, 3H), 4.05-3.76 (m, 2H), 3.70-3.54 (m, 1H), 2.40-2.22 (m, 1H), 2.15-1.67 (m, 6H), 1.66-1.13 (m, 5H). 19F NMR (282 MHz, Chloroform-d) δ−122.87, −130.00.

### Example 49

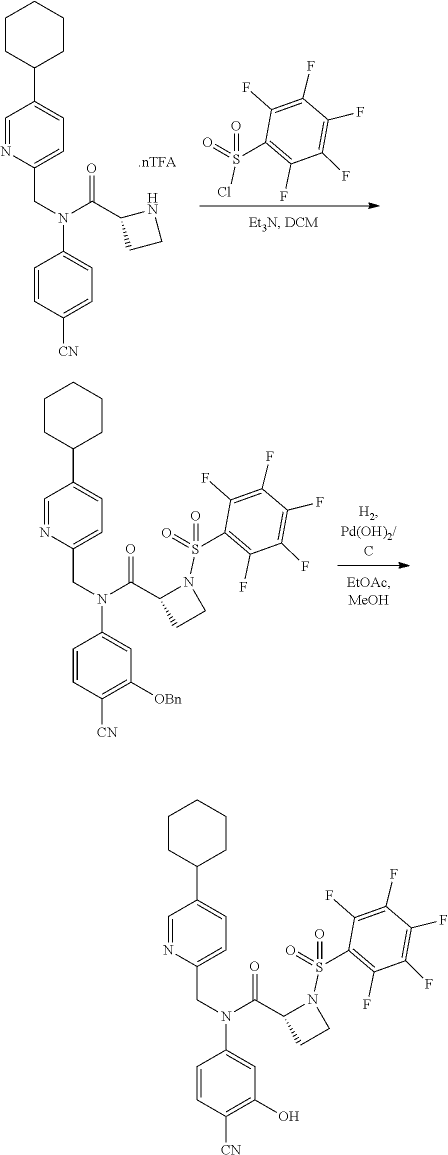

### (R)—N-(4-cyano-3-hydroxyphenyl)-N-((5-cyclohexylpyridin-2-yl)methyl)-1-((perfluorophenyl)sulfonyl)azetidine-2-carboxamide

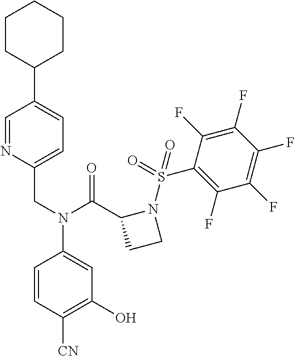 [SMILES: N#Cc1ccc(N(Cc2ccc(C3CCCCC3)cn2)C(=O)[C@H]2CCN2S(=O)(=O)c2c(F)c(F)c(F)c(F)c2F)cc1O]

Step 1. Preparation by a similar procedure to Example 38, step 6, starting from (R)—N-(3-(benzyloxy)-4-cyanophenyl)-N-((5-cyclohexylpyridin-2-yl)methyl)azetidine-2-carb oxamide (264 mg of TFA salt) to obtain (R)—N-(3-(benzyloxy)-4-cyanophenyl)-N-((5-cyclohexylpyridin-2-yl)methyl)-1-((perfluorophenyl)sulfonyl)azetidine-2-carboxamide (159 mg, 80% two steps, i.e., from t-Boc protected azetidine, see Example 4). 1H NMR (300 MHz, Chloroform-d) δ 10.13-9.91 (bs, 1H), 8.55 (s, 1H), 8.14 (d, J=7.9 Hz, 1H), 7.70-7.60 (m, 2H), 7.45-7.34 (m, 5H), 7.17 (s, 1H), 6.94 (d, J=7.9 Hz, 1H), 5.58 (d, J=16.3 Hz, 1H), 5.37-5.04 (m, 4H), 3.98-3.77 (m, 2H), 2.81-2.66 (m, 1H), 2.59-2.41 (m, 1H), 2.00-1.73 (m, 6H), 1.54-1.20 (m, 5H).

Step 2. Preparation by a similar procedure to Example 1, step 8, starting from (R)—N-(3-(benzyloxy)-4-cyanophenyl)-N-((5-cyclohexylpyridin-2-yl)methyl)-1-((perfluorophenyl)sulfonyl)azetidine-2-carboxamide (159 mg, 0.224 mmol) to obtain (R)—N-(4-cyano-3-hydroxyphenyl)-N-((5-cyclohexylpyridin-2-yl)methyl)-1-((perfluorophenyl)sulfonyl)azetidine-2-carboxamide (74 mg, 53%). 1H NMR (300 MHz, Chloroform-d) δ 8.27 (d, J=2.0 Hz, 1H), 7.73-7.63 (m, 1H), 7.62-7.51 (m, 1H), 7.31-7.15 (m, 1H), 6.65 (d, J=8.5 Hz, 1H), 6.51 (s, 1H), 5.20 (d, J=14.4 Hz, 1H), 4.96 (t, J=7.8 Hz, 1H), 4.69 (d, J=14.5 Hz, 1H), 4.22-4.03 (m, 2H), 2.60-2.45 (m, 1H), 2.32-2.16 (m, 1H), 2.14-2.02 (m, 1H), 1.91-1.67 (m, 5H), 1.50-1.16 (m, 5H). 19F NMR (282 MHz, Chloroform-d) δ−135.66, −146.46, −159.18.

### Example 50

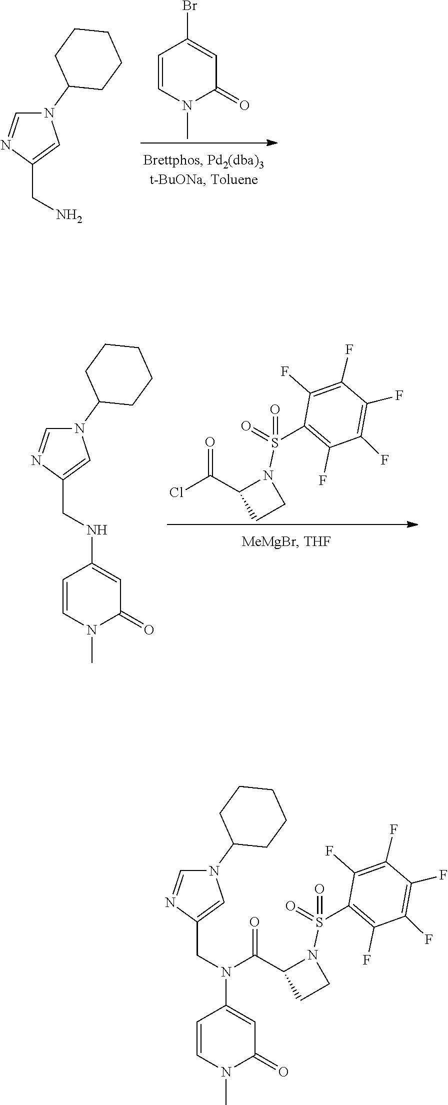

### (R)—N-((1-cyclohexyl-1H-imidazol-4-yl)methyl)-N-(1-methyl-2-oxo-1,2-dihydropyridin-4-yl)-1-((perfluorophenyl)sulfonyl)azetidine-2-carboxamide

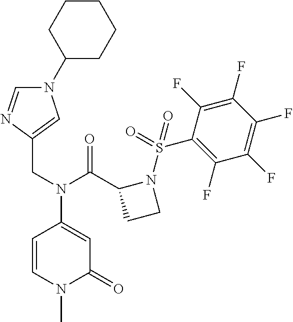 [SMILES: Cn1ccc(N(Cc2cn(C3CCCCC3)cn2)C(=O)[C@H]2CCN2S(=O)(=O)c2c(F)c(F)c(F)c(F)c2F)cc1=O]

Step 1. Under argon, to (1-cyclohexyl-1H-imidazol-4-yl)methanamine (484.6, 2.70 mmol) were added 4-bromo-1-methylpyridin-2(1H)-one (442 mg, 2.35 mmol), Brettphos (63 mg, 5 mol %) and Pd2(dba)3 (26.8 mg, 1.25 mol %). The mixture was thoroughly flushed with argon, and then toluene (30 mL) and 0.8M in THF sodium tert-butoxide (3.7 mL, 2.96 mmol) were added. The mixture was heated at 100° C. for 18 hours. After cooling, the mixture was poured onto cold aqueous ammonium chloride. The mixture was extracted with ethyl acetate (2×). The extract was washed with brine, dried (Na2SO4) and concentrated. Purification by flash column chromatography (85:15:1 DCM/MeOH/NH4OH) gave 4-(((1-cyclohexyl-1H-imidazol-4-yl)methyl)amino)-1-methylpyridin-2(1H)-one (192 mg, 24%). 1H NMR (300 MHz, Chloroform-d) δ 7.50 (d, J=1.4 Hz, 1H), 7.04-6.88 (m, 2H), 5.69-5.58 (m, 2H), 4.83-4.72 (m, 1H), 4.16 (d, J=6.1 Hz, 2H), 3.88 (tt, J=11.7, 3.9 Hz, 1H), 3.43 (s, 3H), 2.16-2.03 (m, 2H), 1.98-1.72 (m, 3H), 1.72-1.14 (m, 5H).

Step 2. Preparation by a similar procedure to Example 22, step 1, starting from 4-(((1-cyclohexyl-1H-imidazol-4-yl)methyl)amino)-1-methylpyridin-2(1H)-one (152 mg, 0.45 mmol) to obtain (R)—N-((1-cyclohexyl-1H-imidazol-4-yl)methyl)-N-(1-methyl-2-oxo-1,2-dihydropyridin-4-yl)-1-((perfluorophenyl)sulfonyl)azetidine-2-carboxamide (9 mg after collection of only pure fraction from column chromatography, and then preparative TLC (DCM/MeOH 96:4)). 1H NMR (300 MHz, Chloroform-d) δ 7.43 (s, 1H), 7.37-7.30 (m, 1H), 6.90 (s, 1H), 6.29-6.18 (m, 2H), 5.20-5.10 (m, 1H), 4.75 (d, J=15.2 Hz, 1H), 4.63 (d, J=15.2 Hz, 1H), 4.26-4.00 (m, 2H), 3.85 (tt, J=11.8, 3.8 Hz, 1H), 3.54 (s, 3H), 2.39-2.25 (m, 2H), 2.14-1.70 (m, 5H), 1.67-1.16 (m, 5H). 19F NMR (282 MHz, Chloroform-d) δ−135.88, −146.68, −159.62.

### Example 51

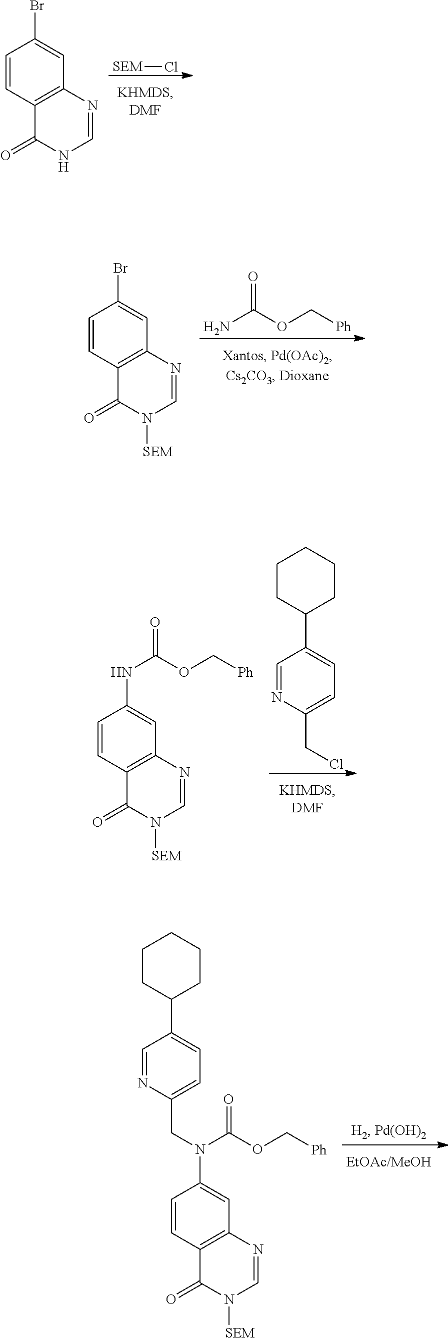

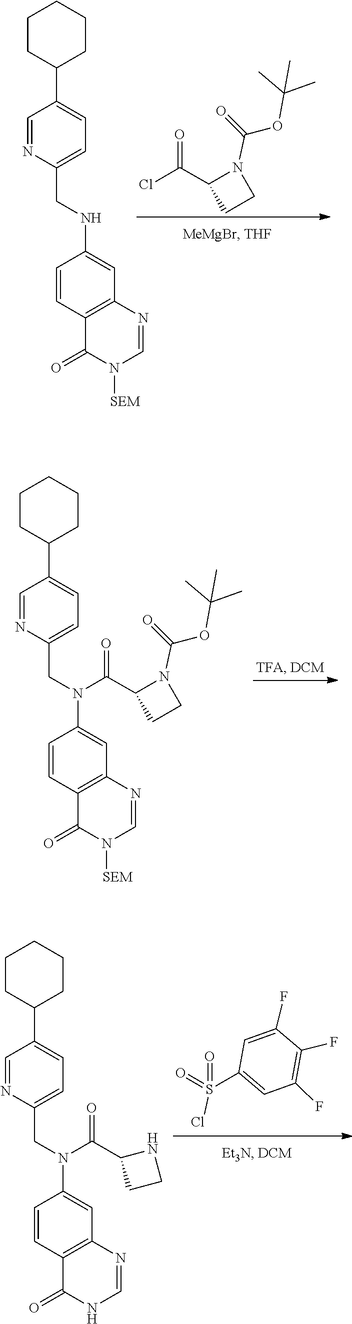 [SMILES: C[Si](C)(C)CCOCn1cnc2cc(NCc3ccc(C4CCCCC4)cn3)ccc2c1=O ; CC(C)(C)OC(=O)N1CC[C@@H]1C(=O)Cl ; CC(C)(C)OC(=O)N1CC[C@@H]1C(=O)N(Cc1ccc(C2CCCCC2)cn1)c1ccc2c(=O)n(COCC[Si](C)(C)C)cnc2c1 ; O=C([C@H]1CCN1)N(Cc1ccc(C2CCCCC2)cn1)c1ccc2c(=O)[nH]cnc2c1 ; O=S(=O)(Cl)c1cc(F)c(F)c(F)c1]

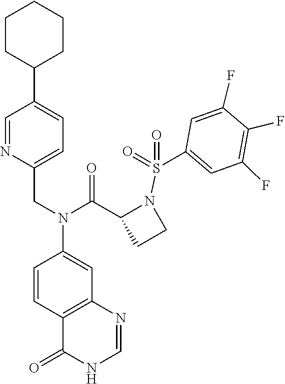 [SMILES: O=C([C@H]1CCN1S(=O)(=O)c1cc(F)c(F)c(F)c1)N(Cc1ccc(C2CCCCC2)cn1)c1ccc2c(=O)[nH]cnc2c1]

### (R)-1-((3-cyano-4,5-difluorophenyl)sulfonyl)-N-((5-cyclohexylpyridin-2-yl)methyl)-N-(4-oxo-3,4-dihydroquinazolin-7-yl)azetidine-2-carboxamide

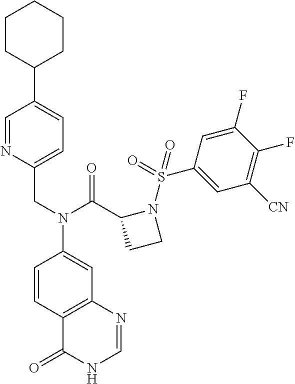 [SMILES: N#Cc1cc(S(=O)(=O)N2CC[C@@H]2C(=O)N(Cc2ccc(C3CCCCC3)cn2)c2ccc3c(=O)[nH]cnc3c2)cc(F)c1F]

Step 1. Preparation by a similar procedure to Example 2, step 1, starting from 7-bromoquinazolin-4(3H)-one (1.0 g, 4.46 mmol) to obtain 7-bromo-3-((2-(trimethylsilyl)ethoxy)methyl)quinazolin-4(3H)-one (755 mg, 48% yield; the rest of material was mainly starting material). 1H NMR (300 MHz, Chloroform-d) δ 8.21-8.15 (m, 2H), 7.92 (d, J=1.9 Hz, 1H), 7.65 (dd, J=8.6, 1.9 Hz, 1H), 5.44 (s, 2H), 3.75-3.62 (m, 2H), 1.04-0.92 (m, 2H), −0.01 (s, 9H).

Step 2. Preparation by a similar procedure to Example 2, step 2, starting from 7-bromo-3-((2-(trimethylsilyl)ethoxy)methyl)quinazolin-4(3H)-one (755 mg, 2.13 mmol) to obtain benzyl (4-oxo-3-((2-(trimethylsilyl)ethoxy)methyl)-3,4-dihydroquinazolin-7-yl)carbamate (533 mg, 59% yield). 1H NMR (300 MHz, Chloroform-d) δ 8.27 (d, J=8.7 Hz, 1H), 8.14 (s, 1H), 7.73 (d, J=2.2 Hz, 1H), 7.63 (dd, J=8.7, 2.2 Hz, 1H), 7.49-7.34 (m, 5H), 7.05 (bs, 1H), 5.43 (s, 2H), 5.26 (s, 2H), 3.75-3.62 (m, 2H), 1.04-0.91 (m, 2H), 0.00 (s, 9H).

Step 3. Preparation by a similar procedure to Example 2, step 3, starting from benzyl (4-oxo-3-((2-(trimethylsilyl)ethoxy)methyl)-3,4-dihydroquinazolin-7-yl)carbamate (530 mg, 1.24 mmol) to obtain benzyl ((5-cyclohexylpyridin-2-yl)methyl)(4-oxo-3-((2-(trimethylsilyl)ethoxy)methyl)-3,4-dihydroquinazolin-7-yl)carbamate (625 mg, 84% yield) as a yellow oil. 1H NMR (300 MHz, Chloroform-d) δ 8.42 (d, J=2.3 Hz, 1H), 8.25 (dd, J=8.7, 0.5 Hz, 1H), 8.13 (s, 1H), 7.69-7.55 (m, 2H), 7.46 (dd, J=8.1, 2.3 Hz, 1H), 7.37-7.14 (m, 6H), 5.43 (s, 2H), 5.23 (s, 2H), 5.10 (s, 2H), 3.76-3.61 (m, 2H), 2.60-2.46 (m, 1H), 1.97-1.70 (m, 5H), 1.52-1.27 (m, 5H), 1.03-0.91 (m, 2H), −0.01 (s, 9H).

Step 4. Preparation by a similar procedure to Example 2, step 4, starting from benzyl ((5-cyclohexylpyridin-2-yl)methyl)(4-oxo-3-((2-(trimethylsilyl)ethoxy)methyl)-3,4-dihydroquinazolin-7-yl)carbamate (624 mg, 1.04 mmol) to obtain 7-(((5-cyclohexylpyridin-2-yl)methyl)amino)-3-((2-(trimethylsilyl)ethoxy)methyl)quinazolin-4(3H)-one (417 mg, 86% yield) of a white solid. 1H NMR (300 MHz, Chloroform-d) δ 8.46 (d, J=2.3, 1H), 8.15-8.04 (m, 2H), 7.53 (dd, J=8.1, 2.3 Hz, 1H), 7.32-7.19 (m, 1H), 6.87 (dd, J=8.7, 2.3 Hz, 1H), 6.77 (d, J=2.3 Hz, 1H), 5.67 (bs, 1H), 5.40 (s, 2H), 4.53 (d, J=4.9 Hz, 2H), 3.73-3.61 (m, 2H), 2.63-2.49 (m, 1H), 1.97-1.59 (m, 5H), 1.53-1.21 (m, 5H), 1.02-0.88 (m, 2H), 0.00 (s, 9H).

Step 5. Preparation by a similar procedure to Example 38, step 4, starting from 7-(((5-cyclohexylpyridin-2-yl)methyl)amino)-3-((2-(trimethylsilyl)ethoxy)methyl)quinazolin-4(3H)-one (151.4 mg, 0.326 mmol) to obtain tert-butyl (R)-2-(((5-cyclohexylpyridin-2-yl)methyl)(4-oxo-3 (trimethylsilyl)ethoxy)methyl)-3,4-di hydroquinazolin-7-yl)carbamoyl)azetidine-1-carboxylate (179 mg, 85% yield) as a yellow oil. 1H NMR (300 MHz, Chloroform-d) δ 8.37-8.27 (m, 2H), 8.18 (s, 1H), 7.61-7.41 (m, 4H), 5.44 (s, 2H), 5.19-5.05 (m, 2H), 4.75-4.58 (m, 1H), 4.21-4.03 (m, 1H), 3.82-3.61 (m, 3H), 2.57-2.44 (m, 1H), 2.32-2.14 (m, 2H), 1.91-1.71 (m, 5H), 1.51-1.23 (m, 5H), 1.04-0.92 (m, 2H), 0.02 (s, 9H).

Step 6. Preparation by a similar procedure to Example 38, step 5, starting from tert-butyl (R)-2-(((5-cyclohexylpyridin-2-yl)methyl)(4-oxo-3 (trimethylsilyl)ethoxy)methyl)-3,4-di hydroquinazolin-7-yl)carbamoyl)azetidine-1-carboxylate (178.6 mg, 0.276 mmol) to obtain after flash column chromatography (80:20:1 DCM:MeOH:NH4OH) tert-butyl (R)-2-(((5-cyclohexylpyridin-2-yl)methyl)(4-oxo-3 (trimethylsilyl)ethoxy)methyl)-3,4-di hydroquinazolin-7-yl)carbamoyl)azetidine-1-carboxylate TFA salt (50 mg, 34% yield as TFA salt) as a white solid. 1H NMR (300 MHz, Chloroform-d) δ 8.38 (d, J=2.2 Hz, 1H), 8.16 (d, J=8.5 Hz, 1H), 7.85 (s, 1H), 7.61-7.51 (m, 1H), 7.50-7.37 (m, 2H), 7.30-7.23 (m, 1H), 5.10 (s, 2H), 4.60-4.49 (m, 1H), 3.71-3.60 (m, 1H), 3.53 (q, J=7.9 Hz, 1H), 2.62-2.45 (m, 2H), 2.43-2.27 (m, 1H), 1.94-1.74 (m, 5H), 1.51-1.32 (m, 5H).

Step 7. To a solution of tert-butyl (R)-2-(((5-cyclohexylpyridin-2-yl)methyl)(4-oxo-3-((2-(trimethylsilyl)ethoxy)methyl)-3,4-di hydroquinazolin-7-yl)carbamoyl)azetidine-1-carboxylate TFA salt (49.5 mg, 0.093 mmol as TFA salt) in DCM (1.6 mL) were added at 0° C. triethylamine (0.039 mL, 0.279 mmol) followed by 3-cyano-4,5-difluorobenzenesulfonyl chloride (20.8 mg, 0.065 mmol, 0.7 equiv) under argon. The mixture was stirred at 0° C. for 1 hour. Additional dichloromethane was added, and the mixture was washed with water, dried (Na2SO4), and concentrated. Purification by preparative TLC (95:5 DCM/MeOH) gave (R)-1-((3-cyano-4,5-difluorophenyl)sulfonyl)-N-((5-cyclohexylpyridin-2-yl)methyl)-N-(4-oxo-3,4-dihydroquinazolin-7-yl)azetidine-2-carboxamide (35 mg, 86% yield calculated based on the sulfonyl chloride as limiting reagent) as a white solid. 1H NMR (300 MHz, Chloroform-d) δ 10.90 (bs, 1H), 8.39 (s, 1H), 8.28 (d, J=8.5 Hz, 1H), 8.22-8.03 (m, 2H), 7.96 (s, 1H), 7.64 -7.54 (m, 2H), 7.46-7.31 (m, 2H), 5.16-4.95 (m, 3H), 4.07-3.93 (m, 1H), 3.76-3.62 (m, 1H), 2.63-2.49 (m, 1H), 2.48-2.33 (m, 1H), 2.03-1.72 (m, 6H), 1.52-1.31 (m, 5H). 19F NMR (282 MHz, Chloroform-d) δ−122.72, −129.86.

### Example 52

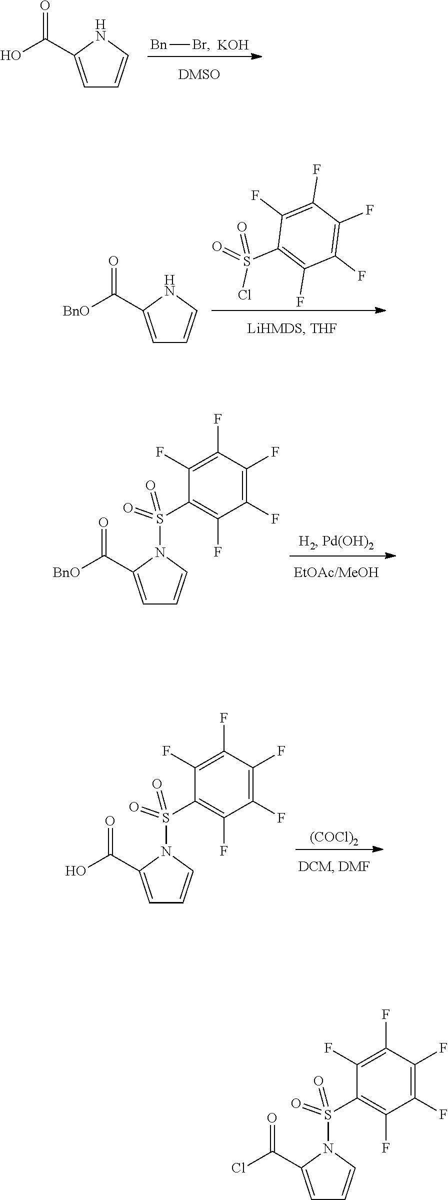

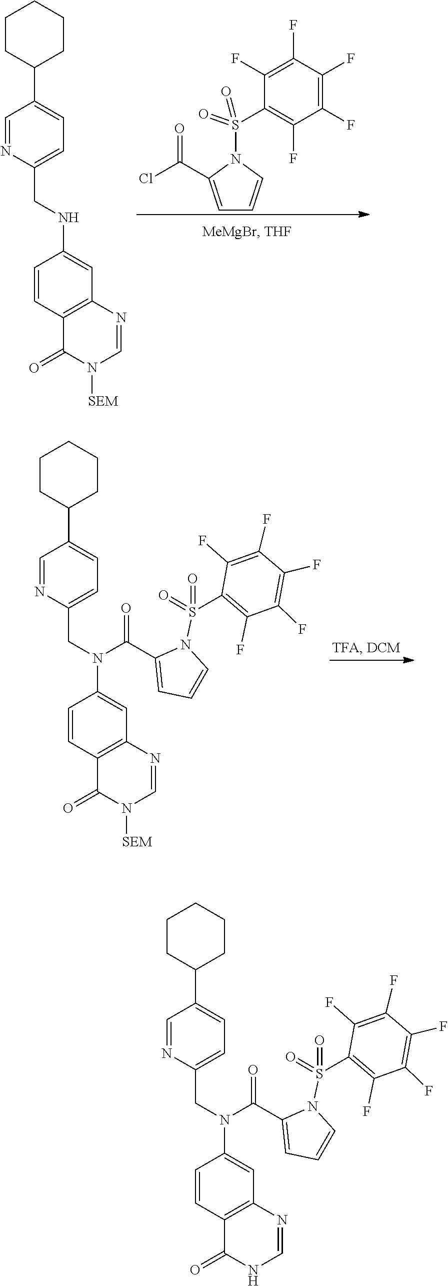 [SMILES: C[Si](C)(C)CCOCn1cnc2cc(NCc3ccc(C4CCCCC4)cn3)ccc2c1=O ; O=C(Cl)c1cccn1S(=O)(=O)c1c(F)c(F)c(F)c(F)c1F ; C[Si](C)(C)CCOCn1cnc2cc(N(Cc3ccc(C4CCCCC4)cn3)C(=O)c3cccn3S(=O)(=O)c3c(F)c(F)c(F)c(F)c3F)ccc2c1=O ; O=C(c1cccn1S(=O)(=O)c1c(F)c(F)c(F)c(F)c1F)N(Cc1ccc(C2CCCCC2)cn1)c1ccc2c(=O)[nH]cnc2c1]

### N-((5-cyclohexylpyridin-2-yl)methyl)-N-(4-oxo-3,4-dihydroquinazolin-7-yl)-1-((perfluorophenyl)sulfonyl)-1H-pyrrole-2-carboxamide

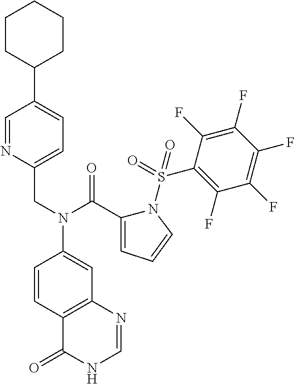 [SMILES: O=C(c1cccn1S(=O)(=O)c1c(F)c(F)c(F)c(F)c1F)N(Cc1ccc(C2CCCCC2)cn1)c1ccc2c(=O)[nH]cnc2c1]

Step 1. To a solution of 1H-pyrrole-2-carboxylic acid (1.15 g, 10.3 mmol) in DMSO (20 mL) was added potassium hydroxide (567 mg, 10.1 mmol) under argon. After attiring for 5-10 minutes, benzyl bromide (1.29 mL, 10.8 mmol) was added. The mixture was stirred at room temperature for 3 hours. The mixture was poured onto water, and was extracted with dichloromethane (2×). The extract was washed with water (2×), dried (Na2SO4), and concentrated. Purification by flash column chromatography (9:1 hexane/EtOAc) gave benzyl 1H-pyrrole-2-carboxylate (1.1 g, 52% yield) as a white solid. 1H NMR (300 MHz, Chloroform-d) δ 9.17 (bs, 1H), 7.50-7.29 (m, 5H), 7.03-6.91 (m, 2H), 6.33-6.23 (m, 1H), 5.33 (s, 2H).

Step 2. To a solution of benzyl 1H-pyrrole-2-carboxylate (200 mg, 0.99 mmol) in THF (5 mL) was added at 0° C. 1M LiHMDS in THF (1.49 mL, 1.49 mmol). After 5-10 minutes, perfluorobenzenesulfonyl chloride (0.16 mL, 1.09 mmol) was added at 0° C. The mixture was allowed to reach room temperature and stirred for 22 hours. Cold aqueous ammonium chloride was added, and the mixture was extracted with ethyl acetate (2×). The extract was washed with brine, dried (Na2SO4), and concentrated. Purification by flash column chromatography (95:5 hexane/EtOAc) gave benzyl 1-((perfluorophenyl)sulfonyl)-1H-pyrrole-2-carboxylate (175 mg, 41% yield). 1H NMR (300 MHz, Chloroform-d) δ 7.76-7.68 (m, 1H), 7.42-7.23 (m, 5H), 7.20-7.13 (m, 1H), 6.38 (t, J=3.5 Hz, 1H), 5.22 (s, 2H).

Step 3. Preparation by a similar procedure to Example 1, step 8, starting from benzyl 1-((perfluorophenyl)sulfonyl)-1H-pyrrole-2-carboxylate (169 mg, 0.39 mmol) to obtain crude 1-((perfluorophenyl)sulfonyl)-1H-pyrrole-2-carboxylic acid (129 mg, 96% yield) as a white solid. 1H NMR (300 MHz, Chloroform-d) δ 7.77 (s, 1H), 7.38-7.18 (m, 1H), 6.42 (t, J=3.6 Hz, 1H).

Step 4. Preparation by a similar procedure to Example 2, step 5a, starting from crude 1-((perfluorophenyl)sulfonyl)-1H-pyrrole-2-carboxylic acid (128.5 mg, 0.38 mmol) to obtain 1-((perfluorophenyl)sulfonyl)-1H-pyrrole-2-carbonyl chloride (134 mg, 99% yield). 1H NMR (300 MHz, Chloroform-d) δ 7.97-7.89 (m, 1H), 7.61-7.51 (m, 1H), 6.56-6.46 (m, 1H).

Step 5. Preparation by a similar procedure to Example 22, step 1, starting from 7-(((5-cyclohexylpyridin-2-yl)methyl)amino)-3-((2-(trimethylsilyl)ethoxy)methyl)quinazolin-4(3H)-one (84.9 mg, 0.182 mmol) and 1-((perfluorophenyl)sulfonyl)-1H-pyrrole-2-carbonyl chloride (131.4 mg, 0.365 mmol) to obtain

N-((5-cyclohexylpyridin-2-yl)methyl)-N-(4-oxo-3-((2-(trimethylsilyl)ethoxy)methyl)-3,4-dihydroquinazolin-7-yl)-1-((perfluorophenyl)sulfonyl)-1H-pyrrole-2-carboxamide (32 mg, 22% yield). 1H NMR (300 MHz, Chloroform-d) δ 8.45-8.28 (m, 1H), 8.20-8.11 (m, 2H), 7.67-7.63 (m, 1H), 7.61-7.54 (m, 1H), 7.50-7.45 (m, 1H), 7.44-7.42 (m, 1H), 7.41-7.34 (m, 1H), 6.07-6.00 (m, 1H), 5.99-5.94 (m, 1H), 5.39 (s, 2H), 5.23 (s, 2H), 3.73-3.60 (m, 2H), 2.57-2.45 (s, 1H), 1.93-1.71 (m, 6H), 1.50-1.31 (m, 4H), 1.02-0.83 (m, 2H), −0.07 (m, 9H). 19F NMR (282 MHz, Chloroform-d) δ−134.91, −143.73, −158.67 (m).

Step 6. To a solution of N-((5-cyclohexylpyridin-2-yl)methyl)-N-(4-oxo-3-((2-(trimethylsilyl)ethoxy)methyl)-3,4-dihydroquinazolin-7-yl)-1-((perfluorophenyl)sulfonyl)-1H-pyrrole-2-carboxamide (27.7 mg, 0.035 mmol) in dichloromethane (0.15 mL) was added trifluoroacetic acid (0.15 mL) under argon. The mixture was stirred for 2 hours. Additional dichloromethane was added, and then the mixture was poured onto cold aqueous sodium bicarbonate. The pH of aqueous phase was 7-8. The mixture was extracted with dichloromethane (2×). The extract was washed with additional aqueous sodium bicarbonate, dried (Na2SO4), and concentrated. Purification by preparative TLC (3:7 hexane/EtOAc with 5% MeOH) gave N-((5-cyclohexylpyridin-2-yl)methyl)-N-(4-oxo-3,4-dihydroquinazolin-7-yl)-1-((perfluorophenyl)sulfonyl)-1H-pyrrole-2-carboxamide (16.1 mg, 70% yield) as a white foam. 1H NMR (300 MHz, Chloroform-d) δ 10.99 (bs, 1H), 8.33 (s, 1H), 8.12 (d, J=8.6 Hz, 1H), 7.92 (s, 1H), 7.72-7.65 (m, 1H), 7.64-7.30 (m, 4H), 6.08-5.93 (m, 2H), 5.22 (s, 2H), 2.60-2.43 (m, 1H), 1.91-1.57 (m, 5H), 1.52-1.19 (m, 5H). 19F NMR (282 MHz, Chloroform-d) δ−134.87, −143.68, −158.52.

### Example 53

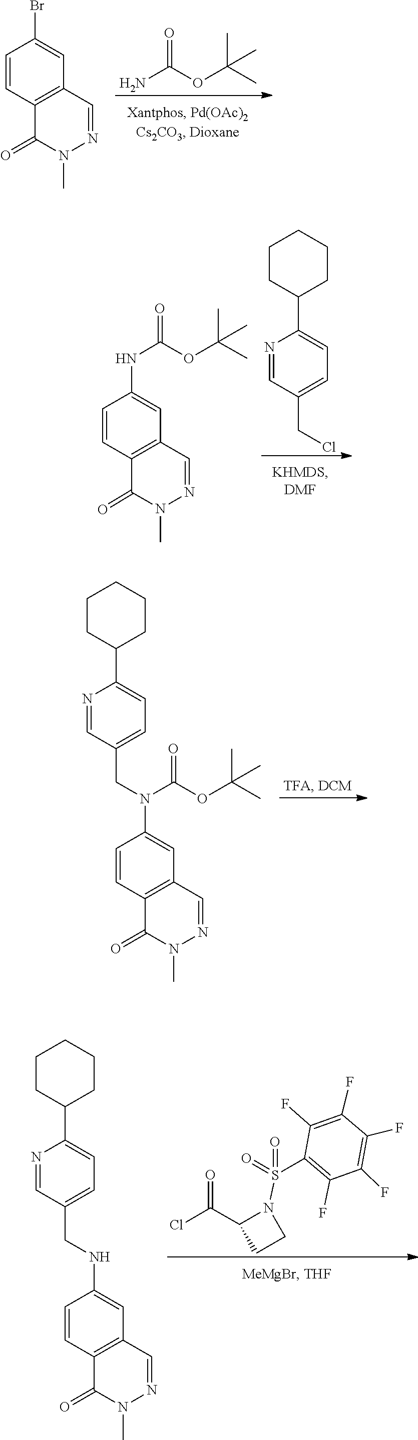

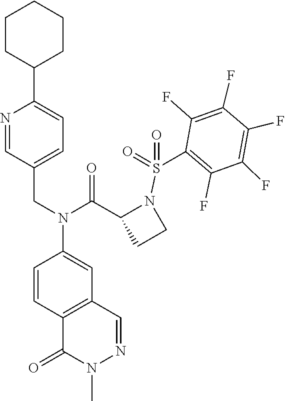 [SMILES: Cn1ncc2cc(N(Cc3ccc(C4CCCCC4)nc3)C(=O)[C@H]3CCN3S(=O)(=O)c3c(F)c(F)c(F)c(F)c3F)ccc2c1=O]

### (R)—N-((6-cyclohexylpyridin-3-yl)methyl)-N-(2-methyl-1-oxo-1,2-dihydrophthalazin-6-yl)-1-((perfluorophenyl)sulfonyl)azetidine-2-carboxamide

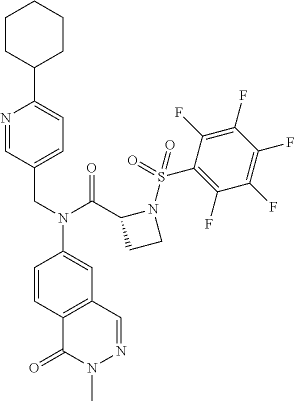 [SMILES: Cn1ncc2cc(N(Cc3ccc(C4CCCCC4)nc3)C(=O)[C@H]3CCN3S(=O)(=O)c3c(F)c(F)c(F)c(F)c3F)ccc2c1=O]

Step 1. Preparation by a similar procedure to Example 10, step 2, starting from 6-bromo-2-methylphthalazin-1(2H)-one (926 mg, 3.87 mmol) to obtain tert-butyl (2-methyl-1-oxo-1,2-dihydrophthalazin-6-yl)carbamate (916 mg, 86% yield) as a white solid. 1H NMR (300 MHz, Chloroform-d) δ 8.35 (d, J=8.7 Hz, 1H), 8.15-8.05 (m, 2H), 7.45 (dd, J=8.7, 2.3 Hz, 1H), 6.95 (bs, 1H), 3.86 (s, 3H), 1.56 (s, 9H).

Step 2. Preparation by a similar procedure to Example 7, step 3, starting from tert-butyl (2-methyl-1-oxo-1,2-dihydrophthalazin-6-yl)carbamate (300 mg, 1.09 mmol) and 5-(chloromethyl)-2-cyclohexylpyridine (3.2 mL of a 0.5M solution in toluene, 1.6 mmol) to obtain tert-butyl ((6-cyclohexylpyridin-3-yl)methyl)(2-methyl-1-oxo-1,2-dihydrophthalazin-6-yl)carbamate (492 mg, 99% yield) as a yellow oil. 1H NMR (300 MHz, Chloroform-d) δ 8.46-8.31 (m, 2H), 8.07 (d, J=0.7 Hz, 1H), 7.60 (dd, J=8.6, 2.2 Hz, 1H), 7.54-7.45 (m, 2H), 7.14 (d, J=8.1 Hz, 1H), 4.95 (s, 2H), 3.85 (s, 3H), 2.77-2.63 (m, 1H), 1.99-1.70 (m, 5H), 1.46 (s, 9H), 1.57-1.32 (m, 5H).

Step 3. Preparation by a similar procedure to Example 7, step 4, starting from tert-butyl ((6-cyclohexylpyridin-3-yl)methyl)(2-methyl-1-oxo-1,2-dihydrophthalazin-6-yl)carbamate (492 mg, 1/1 mmol) to obtain 6-(((6-cyclohexylpyridin-3-yl)methyl)amino)-2-methylphthalazin-1(2H)-one (322 mg, 84% yield). 1H NMR (300 MHz, Chloroform-d) δ 8.59 (dd, J=2.3, 0.9 Hz, 1H), 8.22 (dd, J=8.8, 0.6 Hz, 1H), 7.96 (s, 1H), 7.67 (dd, J=8.1, 2.3 Hz, 1H), 7.21 (d, J=8.1 Hz, 2H), 7.03 (ddd, J=8.8, 2.4, 0.6 Hz, 1H), 6.63 (d, J=2.4 Hz, 1H), 4.75 (bs, 1H), 4.46 (s, 2H), 3.80 (s, 3H), 2.82-2.67 (m, 1H), 2.00-1.70 (m, 5H), 1.61-1.25 (m, 5H).

Step 4. Preparation by a similar procedure to Example 22, step 1, starting from 6-(((6-cyclohexylpyridin-3-yl)methyl)amino)-2-methylphthalazin-1(2H)-one (322 mg, 0.925 mmol) to obtain after flash column chromatography (3:7 hexane/EtOAc with 3% MeOH) (R)—N-((6-cyclohexylpyridin-3-yl)methyl)-N-(2-methyl-1-oxo-1,2-dihydrophthalazin-6-yl)-1-((perfluorophenyl)sulfonyl)azetidine-2-carboxamide (260 mg, from which 40 mg were further purified by preparative TLC (3:7 hexane/EtOAc) to obtain 23 mg) as a white foam. 1H NMR (300 MHz, Chloroform-d) δ 8.46 (d, J=8.3 Hz, 1H), 8.17 (s, 1H), 8.07 (s, 1H), 7.49 (d, J=7.7 Hz, 1H), 7.43-7.32 (m, 2H), 7.13 (d, J=8.3 Hz, 1H), 5.04-4.87 (m, 2H), 4.80 (d, J=14.6 Hz, 1H), 4.22-3.97 (m, 2H), 3.88 (s, 3H), 2.77-2.56 (m, 1H), 2.39-2.17 (m, 1H), 2.09-1.74 (m, 6H), 1.57-1.16 (m, 5H). 19F NMR (282 MHz, Chloroform-d) δ−135.87, −146.28, −159.18.

### Example 54

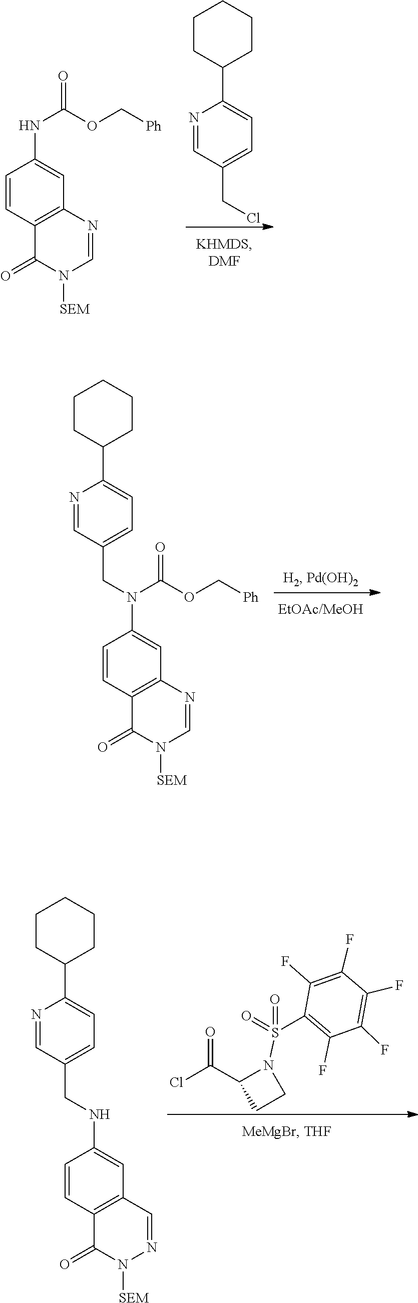

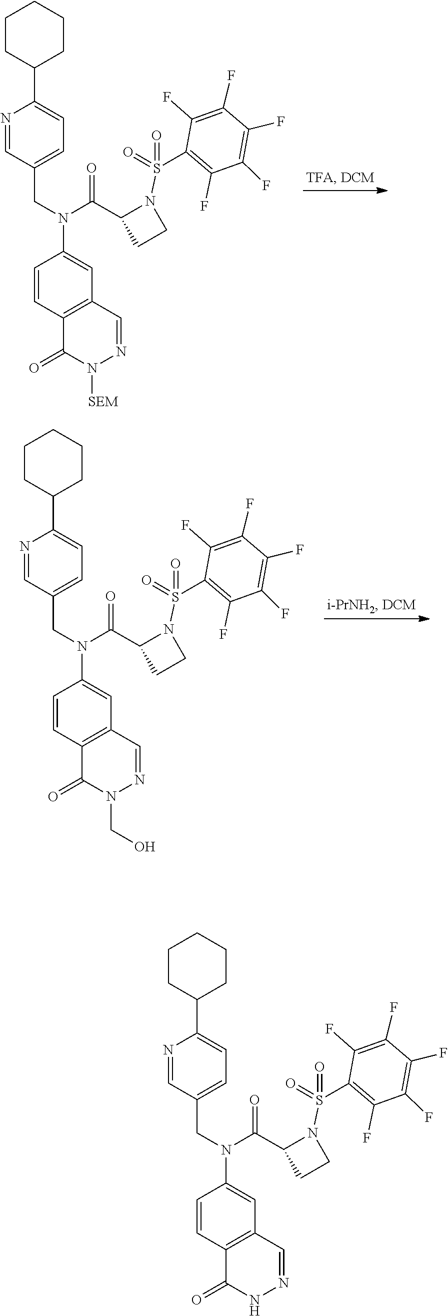

(R)—N-((6-cyclohexylpyridin-3-yl)methyl)-N-(1-oxo-1,2-dihydrophthalazin-6-yl)-1-((perfluorophenyesulfonyl)azetidine-2-carboxamide, also referred to herein as “H172”.

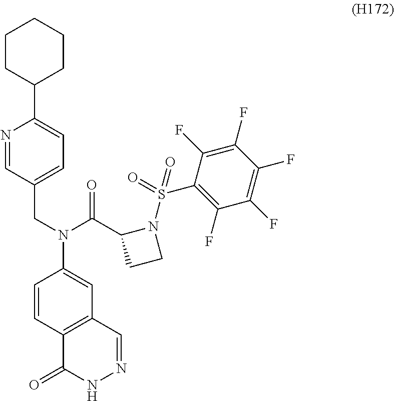 [SMILES: O=C([C@H]1CCN1S(=O)(=O)c1c(F)c(F)c(F)c(F)c1F)N(Cc1ccc(C2CCCCC2)nc1)c1ccc2c(=O)[nH]ncc2c1]

Step 1. Preparation by a similar procedure to Example 2, step 3, starting from benzyl (1-oxo-2-((2-(trimethylsilyl)ethoxy)methyl)-1,2-dihydrophthalazin-6-yl)carbamate (370 mg, 0.87 mmol) to obtain benzyl ((6-cyclohexylpyridin-3-yl)methyl)(1-oxo-2-((2-(trimethylsilyl)ethoxy)methyl)-1,2-dihydrophthalazin-6-yl)carbamate (444 mg, 85% yield). 1H NMR (300 MHz, Chloroform-d) δ 8.44-8.34 (m, 2H), 8.07 (s, 1H), 7.58 (dd, J=8.5, 2.1 Hz, 1H), 7.53-7.45 (m, 2H), 7.37-7.31 (m, 3H), 7.29-7.23 (m, 2H), 7.11 (d, J=8.1 Hz, 1H), 5.56 (s, 2H), 5.23 (s, 2H), 5.00 (s, 2H), 3.80-3.67 (m, 2H), 2.77-2.62 (m, 1H), 1.98-1.71 (m, 6H), 1.59-1.32 (m, 4H), 1.05-0.93 (m, 2H), 0.01 (s, 9H).

Step 2. Preparation by a similar procedure to Example 2, step 4, starting from benzyl ((6-cyclohexylpyridin-3-yl)methyl)(1-oxo-2-((2-(trimethylsilyl)ethoxy)methyl)-1,2-dihydrophthalazin-6-yl)carbamate (444 mg, 0.74 mmol) to obtain 6-(((6-cyclohexylpyridin-3-yl)methyl)amino)-2-((2-(trimethylsilyl)ethoxy)methyl)phthalazin-1(2H)-one (291 mg, 85% yield) as a yellow oil. 1H NMR (300 MHz, Chloroform-d) δ 8.59 (d, J=2.3 Hz, 1H), 8.23 (d, J=8.7 Hz, 1H), 8.00 (s, 1H), 7.65 (dd, J=8.1, 2.3 Hz, 1H), 7.20 (d, J=8.1, 1H), 7.02 (dd, J=8.7, 2.4 Hz, 1H), 6.64 (d, J=2.4 Hz, 1H), 5.54 (s, 2H), 4.77 (bs, 1H), 4.46 (d, J=3.9 Hz, 2H), 3.80-3.66 (m, 2H), 2.82-2.68 (m, 1H), 2.01-1.73 (m, 6H), 1.62-1.33 (m, 4H), 1.05-0.93 (m, 2H), 0.00 (s, 9H).

Step 3. Preparation by a similar procedure to Example 22, step 1, starting from 6-(((6-cyclohexylpyridin-3-yl)methyl)amino)-2-((2-(trimethylsilyl)ethoxy)methyl)phthalazin-1(2H)-one (291 mg, 0.627 mmol) to obtain (R)—N-((6-cyclohexylpyridin-3-yl)methyl)-N-(1-oxo-2-((2-(trimethylsilyl)ethoxy)methyl)-1,2-dihydrophthalazin-6-yl)-1-((perfluorophenyl)sulfonyl)azetidine-2-carboxamide (417 mg, 85% yield). 1H NMR (300 MHz, Chloroform-d) δ 8.48 (d, J=8.3 Hz, 1H), 8.18 (d, J=2.3 Hz, 1H), 8.10 (s, 1H), 7.51 (dd, J=8.1, 2.3 Hz, 1H), 7.44-7.33 (m, 2H), 7.15 (d, J=8.1 Hz, 1H), 5.58 (s, 2H), 4.96 (d, J=14.7 Hz, 1H), 5.02-4.87 (m, 1H), 4.81 (d, J=14.7 Hz, 1H), 4.18-4.01 (m, 2H), 3.82-3.70 (m, 2H), 2.74-2.62 (m, 1H), 2.41-2.24 (m, 1H), 2.01-1.32 (m, 11H), 1.08-0.95 (m, 2H), 0.02 (s, 9H).

Step 4. To a solution of (R)—N-((6-cyclohexylpyridin-3-yl)methyl)-N-(1-oxo-2-((2-(trimethylsilyl)ethoxy)methyl)-1,2-dihydrophthalazin-6-yl)-1-((perfluorophenyl)sulfonyl)azetidine-2-carboxamide (417 mg, 0.536 mmol) in dichloromethane (8 mL) was added trifluoroacetic acid (2.6 mL) under argon. The mixture was stirred for 2 h, and then was poured onto aqueous sodium bicarbonate (pH aqueous layer ca. 7-8). The mixture was extracted with DCM (2×). The extract was washed with aqueous sodium bicarbonate, dried (Na2SO4), and concentrated. Purification by flash column chromatography (3:7 hexane/EtOAc with 4% MeOH) gave (R)—N-((6-cyclohexylpyridin-3-yl)methyl)-N-(2-(hydroxymethyl)-1-oxo-1,2-dihydrophthalazin-6-yl)-1-((perfluorophenyl)sulfonyl)azetidine-2-carboxamide (312 mg, 86% yield). 1H NMR (300 MHz, Chloroform-d) δ 8.51-8.43 (m, 1H), 8.23-8.17 (m, 1H), 8.12 (s, 1H), 7.52 (dd, J=8.0, 2.5 Hz, 1H), 7.4-7.37 (m, 2H), 7.16 (d, J=8.0 Hz, 1H), 5.66 (s, 2H), 4.97 (d, J=14.8 Hz, 1H), 5.05-4.87 (m, 1H), 4.83 (d, J=14.8 Hz, 1H), 4.21-4.03 (m, 2H), 2.76-2.63 (m, 1H), 2.37-2.24 (m, 1H), 2.03-1.31 (m, 11H).

Step 5. To a solution of (R)—N-((6-cyclohexylpyridin-3-yl)methyl)-N-(2-(hydroxymethyl)-1-oxo-1,2-dihydrophthalazin-6-yl)-1-((perfluorophenyl)sulfonyl)azetidine-2-carboxamide (311.5 mg, 0.46 mmol) in dichloromethane (5 mL) was added at −10° C. isopropylamine (0.082 mL, 0.92 mmol) under argon. The mixture was stirred at 0° C. for 24 h. A solution of 10% HOAc/NaOAc in water (1.7 mL) was added at 0° C., and the mixture was extracted with dichloromethane (2×). The extract was washed with water, dried (Na2SO4) and concentrated. Purification by flash column chromatography (6:4 hexane/EtOAc) gave (R)—N-((6-cyclohexylpyridin-3-yl)methyl)-N-(1-oxo-1,2-dihydrophthalazin-6-yl)-1-((perfluorophenyl)sulfonyl)azetidine-2-carboxamide (209 mg, from which 41 mg were further purified by preparative TLC (6:4 hexane/EtOAc) to obtain 30 mg) as a white solid. 1H NMR (300 MHz, Chloroform-d) δ 10.42 (s, 1H), 8.45 (d, J=8.6 Hz, 1H), 8.18 (d, J=2.3 Hz, 1H), 8.13-8.03 (m, 1H), 7.51 (dd, J=8.0, 2.3 Hz, 1H), 7.45-7.37 (m, 2H), 7.15 (d, J=8.0 Hz, 1H), 5.02-4.76 (m, 3H), 4.19-4.03 (m, 2H), 2.67 (m, 1H), 2.38-2.23 (m, 1H), 2.02-1.30 (m, 11H). 19F NMR (282 MHz, Chloroform-d) δ−135.86, −146.21, −159.14.

(R)—N-((6-cyclohexylpyridin-3-yl)methyl)-N-(1-oxo-1,4-dihydrophthalazin-6-yl)-1-((perfluorophenyesulfonyl)azetidine-2-carboxamide, also referred to herein as “H182”

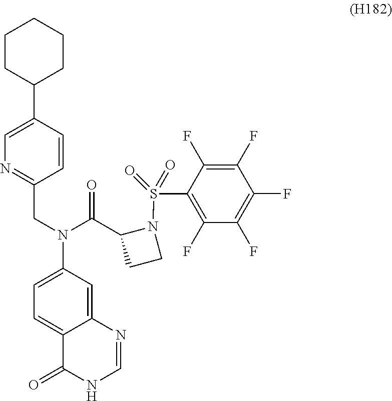 [SMILES: O=C([C@H]1CCN1S(=O)(=O)c1c(F)c(F)c(F)c(F)c1F)N(Cc1ccc(C2CCCCC2)cn1)c1ccc2c(=O)[nH]cnc2c1]

Compound H182 can be made by similar methods as those used to make H172, but starting from benzyl (1-oxo-2-((2-(trimethylsilyl)ethoxy)methyl)-1,4-dihydrophthalazin-6-yl)carbamate instead.

### Example 55

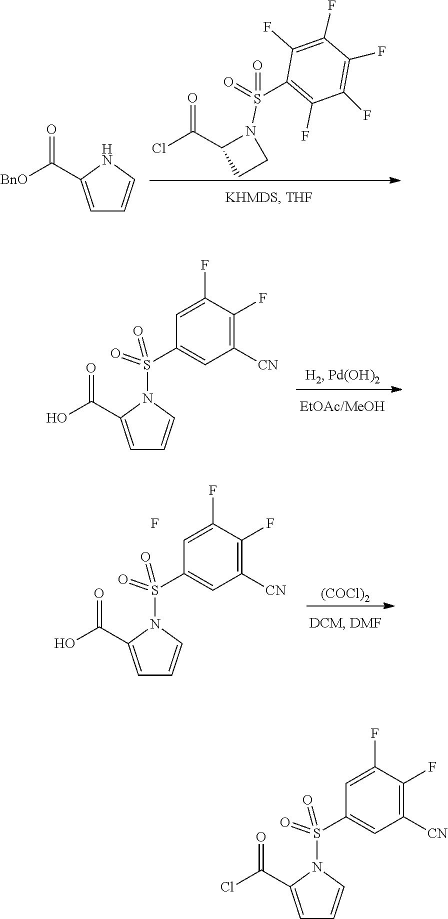

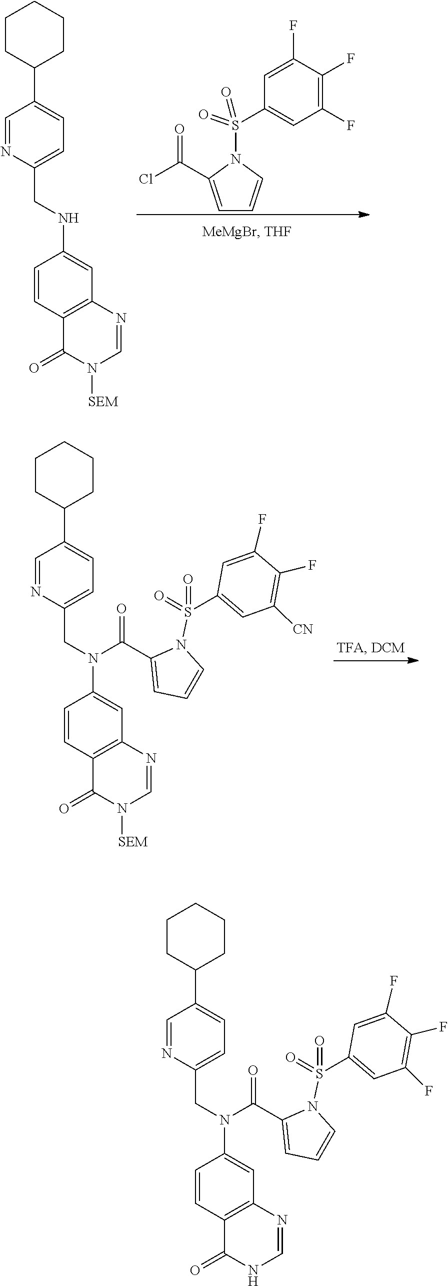 [SMILES: C[Si](C)(C)CCOCn1cnc2cc(NCc3ccc(C4CCCCC4)cn3)ccc2c1=O ; O=C(Cl)c1cccn1S(=O)(=O)c1cc(F)c(F)c(F)c1 ; C[Si](C)(C)CCOCn1cnc2cc(N(Cc3ccc(C4CCCCC4)cn3)C(=O)c3cccn3S(=O)(=O)c3cc(F)c(F)c(C#N)c3)ccc2c1=O ; O=C(c1cccn1S(=O)(=O)c1cc(F)c(F)c(F)c1)N(Cc1ccc(C2CCCCC2)cn1)c1ccc2c(=O)[nH]cnc2c1]

### 1-((3-cyano-4,5-difluorophenyl)sulfonyl)-N-((5-cyclohexylpyridin-2-yl)methyl)-N-(4-oxo-3,4-dihydroquinazolin-7-yl)-1H-pyrrole-2-carboxamide

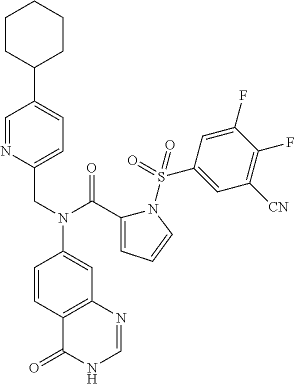 [SMILES: N#Cc1cc(S(=O)(=O)n2cccc2C(=O)N(Cc2ccc(C3CCCCC3)cn2)c2ccc3c(=O)[nH]cnc3c2)cc(F)c1F]

Step 1. To a solution of benzyl 1H-pyrrole-2-carboxylate (301 mg, 1.49 mmol) in THF (7.4 mL) was added at 0° C. 1M KHMDS in THF (2.2 mL, 2.2 mmol). After 5-10 minutes, 3-cyano-4,5-difluorobenzenesulfonyl chloride (525 mg, 1.64 mmol) was added at 0° C. The mixture was allowed to reach room temperature and stirred for 1.5 hours. Cold aqueous ammonium chloride was added, and the mixture was extracted with ethyl acetate (2×). The extract was washed with brine, dried (Na2SO4), and concentrated. Purification by flash column chromatography (90:10 hexane/EtOAc) gave benzyl 1-((3-cyano-4,5-difluorophenyl)sulfonyl)-1H-pyrrole-2-carboxylate (421 mg, 70% yield). 1H NMR (300 MHz, Chloroform-d) δ 8.21-8.02 (m, 2H), 7.71 (dd, J=3.3, 1.8 Hz, 1H), 7.46-7.31 (m, 5H), 7.18 (dd, J=3.7, 1.8 Hz, 1H), 6.46-6.37 (m, 1H), 5.21 (s, 2H).

Step 2. To a stirred solution of benzyl 1-((3-cyano-4,5-difluorophenyl)sulfonyl)-1H-pyrrole-2-carboxylate (421 mg, 1.05 mmol) in ethyl acetate (4.3 mL) and methanol (4.3 mL mL) under nitrogen was added 20% Pd(OH)2 on carbon 50% wet (43 mg). The solution was placed under a hydrogen balloon and stirred for only 3.5 hours. The solution was filtered through Celite®, washed with ethyl acetate and evaporated under reduced pressure. Purification by flash column chromatography (3:7 hexane/ethyl acetate) gave 1-((3-cyano-4,5-difluorophenyl)sulfonyl)-1H-pyrrole-2-carboxylic acid (169 mg, 52% yield) as a yellow solid. 1H NMR (300 MHz, Chloroform-d) δ 8.14-8.05 (m, 2H), 7.81-7.73 (m, 1H), 7.34-7.28 (m, 1H), 6.51-6.42 (m, 1H).

Step 3. Preparation by a similar procedure to Example 2, step 5a, starting from 1-((3-cyano-4,5-difluorophenyl)sulfonyl)-1H-pyrrole-2-carboxylic acid (220 mg, 0.705 mmol) to obtain 1-((3-cyano-4,5-difluorophenyl)sulfonyl)-1H-pyrrole-2-carbonyl chloride (257 mg, 100% yield). 1H NMR (300 MHz, Chloroform-d) δ 8.22-8.10 (m, 1H), 8.16-8.01 (m, 1H), 7.95-7.91 (m, 1H), 7.59-7.55 (m, 1H), 6.57-6.52 (m, 1H).

Step 4. Preparation by a similar procedure to Example 22, step 1, starting from 7-(((5-cyclohexylpyridin-2-yl)methyl)amino)-3-((2-(trimethylsilyl)ethoxy)methyl)quinazolin-4(3H)-one (163 mg, 0.35 mmol) and 1-((3-cyano-4,5-difluorophenyl)sulfonyl)-1H-pyrrole-2-carbonyl chloride (233 mg, 0.705 mmol) to obtain 1-((3-cyano-4,5-difluorophenyl)sulfonyl)-N-((5-cyclohexylpyridin-2-yl)methyl)-N-(4-oxo-3-((2-(trimethylsilyl)ethoxy)methyl)-3,4-dihydroquinazolin-7-yl)-1H-pyrrole-2-carboxamide (153 mg, 58% yield) as a yellow oil. 1H NMR (300 MHz, Chloroform-d) δ 8.42-8.26 (m, 2H), 8.25-8.10 (m, 3H), 7.67 (d, J=2.2 Hz, 1H), 7.57 (dd, J=8.1, 2.2 Hz, 1H), 7.49-7.36 (m, 2H), 7.27-7.24 (m, 1H), 6.12-6.00 (m, 2H), 5.40 (s, 2H), 5.27 (s, 2H), 3.73-3.61 (m, 2H), 2.59-2.46 (m, 1H), 1.96-1.71 (m, 6H), 1.52-1.31 (m, 4H), 1.03-0.90 (m, 2H), 0.00 (s, 9H).

Step 5. Preparation by a similar procedure to Example 52, step 6, starting from 1-((3-cyano-4,5-difluorophenyl)sulfonyl)-N-((5-cyclohexylpyridin-2-yl)methyl)-N-(4-oxo-3-((2-(trimethylsilyl)ethoxy)methyl)-3,4-dihydroquinazolin-7-yl)-1H-pyrrole-2-carboxamide (149.6 mg, 0.197 mmol) to obtain 1-((3-cyano-4,5-difluorophenyl)sulfonyl)-N-((5-cyclohexylpyridin-2-yl)methyl)-N-(4-oxo-3,4-dihydroquinazolin-7-yl)-1H-pyrrole-2-carboxamide (88 mg, 71% yield). 1H NMR (300 MHz, Chloroform-d) δ 11.06 (s, 1H), 8.42-8.35 (s, 1H), 8.34-8.26 (m, 1H), 8.24-8.17 (m, 1H), 8.16-8.10 (m, 1H), 7.97 (s, 1H), 7.70 (d, J=2.0 Hz, 1H), 7.59 (dd, J=7.9, 2.3 Hz, 1H), 7.50-7.43 (m, 1H), 7.42-7.36 (m, 1H), 7.31-7.23 (m, 1H), 6.12-5.99 (m, 2H), 5.27 (s, 2H), 2.60-2.46 (m, 1H), 1.98-1.71 (m, 5H), 1.51-1.23 (m, 5H). MS (ESI+) m/z 629.2 [1\4+H]+.

### Example 56

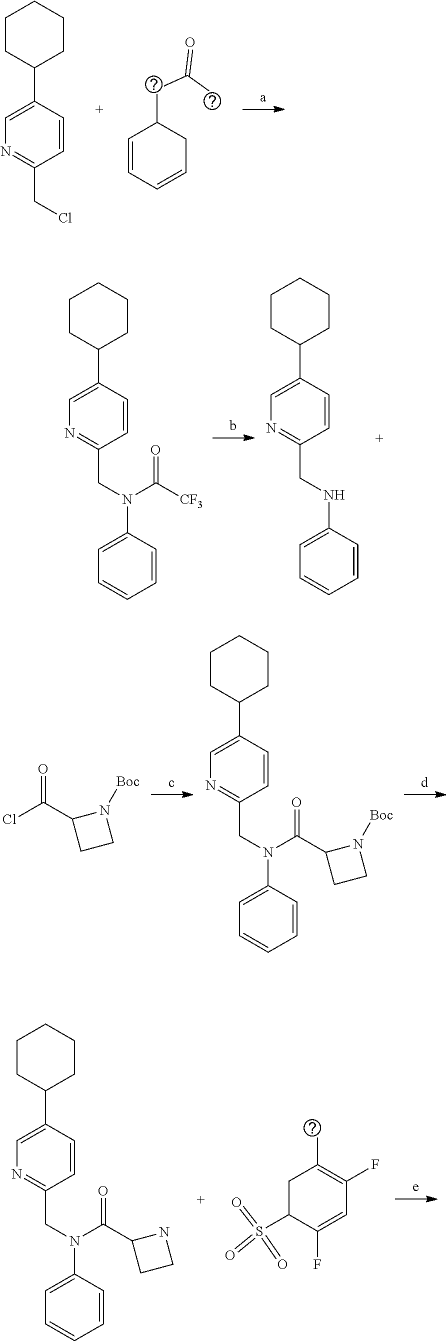

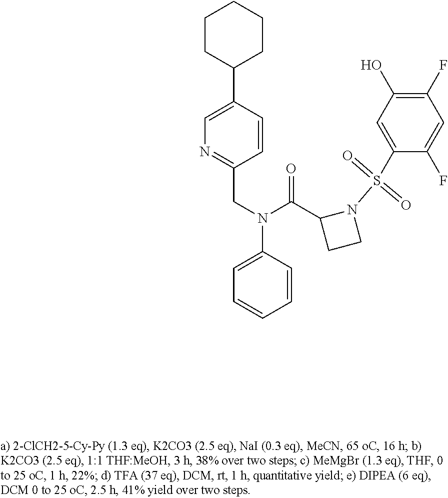 [SMILES: O=C(C1CCN1S(=O)(=O)c1cc(O)c(F)cc1F)N(Cc1ccc(C2CCCCC2)cn1)c1ccccc1]

### (R)-1-((5-cyano-2,4-difluorophenyl)sulfonyl)-N-((5-cyclohexylpyridin-2-yl)methyl)-N-phenylazetidine-2-carboxamide

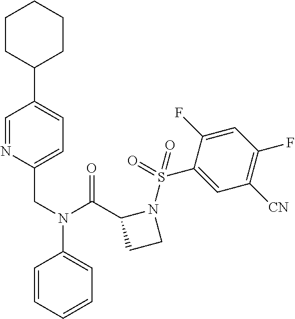 [SMILES: N#Cc1cc(S(=O)(=O)N2CC[C@@H]2C(=O)N(Cc2ccc(C3CCCCC3)cn2)c2ccccc2)c(F)cc1F]

Step 1. To 2,2,2-trifluoro-N-phenylacetamide (1.0 g) under argon was added NaI (236 mg) and K2CO3 (1.828 g). The solids were then dissolved in acetonitrile (35 ml). A 1M solution of (chloromethyl)-5-cyclohexylpyridine (6.8 ml) in toluene was added to the mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. N-((5-cyclohexylpyridin-2-yl)methyl)-2,2,2-trifluoro-N-phenylacetamide was used as a mixture of product and starting material for the next reaction.

Step 2. To crude N-((5-cyclohexylpyridin-2-yl)methyl)-2,2,2-trifluoro-N-phenylacetamide under argon was added K2CO3 (2.0 g) followed by THF (15 ml) and methanol (15 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave N-((5-cyclohexylpyridin-2-yl)methyl)aniline (524 mg, 38% yield over two steps). 1H NMR (300 MHz, CDCl3) δ 8.45 (d, J=2.3 Hz, 1H), 7.50 (dd, J=8.0, 2.3 Hz, 1H), 7.28 (t, J=1.6 Hz, 1H), 7.25-7.16 (m, 2H), 6.78-6.66 (m, 2H), 4.75 (s, 1H), 4.44 (d, J=4.7 Hz, 2H), 2.65-2.45 (m, 1H), 1.96-1.66 (m, 5H), 1.55-1.18 (m, 5H).

Step 3. To a stirred solution of N-((5-cyclohexylpyridin-2-yl)methyl)aniline (524 mg) in THF (8 mL) at 0° C. under argon was added a solution of 1.4M MeMgBr (2.0 mL) in 1:3 toluene:tetrahydrofuran. The reaction mixture was allowed to stir for fifteen minutes before tert-butyl (R)-2-(chlorocarbonyl)azetidine-1-carboxylate (865 mg) in THF (ml) was added. The ice bath was then removed and the reaction was allowed to reach room temperature. After two and a half hours saturated ammonium chloride solution was added. The reaction mixture was then extracted with ethyl acetate. The combined organic extracts were then washed with water, brine, and dried over Na2SO4. Purification by column chromatography (4:6 ethyl acetate/hexanes) gave tert-butyl (R)-2-(((5-cyclohexylpyridin-2-yl)methyl)(phenyl)carbamoyl)azetidine-1-carboxylate (190 mg, 22% yield). 1H NMR (300 MHz, CDCl3) δ 8.33 (d, J=2.1 Hz, 1H), 7.56-7.42 (m, 2H), 7.41-7.28 (m, 3H), 7.15 (d, J=14.8 Hz, 2H), 5.09 (d, J=15.0 Hz, 2H), 4.58 (s, 1H), 4.08 (td, J=8.8, 7.0 Hz, 1H), 3.75 (td, J=8.4, 5.7 Hz, 1H), 2.50 (s, 1H), 2.29-2.07 (m, 2H), 1.97-1.70 (m, 5H), 1.52-1.20 (m, 14H).

Step 4. To a solution of tert-butyl (R)-2-(((5-cyclohexylpyridin-2-yl)methyl)(phenyl)carbamoyl)azetidine-1-carboxylate (85 mg) in DCM (10 ml) under argon was added TFA (1 ml). The reaction mixture was stirred at room temperature for one hour. The reaction was then concentrated and the resulting solid ((R)—N-((5-cyclohexylpyridin-2-yl)methyl)-N-phenylazetidine-2-carboxamide TFA salt) was used directly in the next reaction.

Step 5. The solid ((R)—N-((5-cyclohexylpyridin-2-yl)methyl)-N-phenylazetidine-2-carboxamide TFA salt) was dissolved in DCM (5 ml), and DIPEA (0.193 ml) was added to the solution at 0° C. The reaction mixture was allowed to stir for fifteen minutes. 5-cyano-2,4-difluorobenzenesulfonyl chloride (CAS No. 1807241-08-2) (80 mg) in DCM (5 ml) was added. The ice bath was removed and the reaction was allowed to warm to room temperature. After two and a half hours saturated ammonium chloride solution was added. The reaction mixture was extracted with dichloromethane. The combined organic extracts were washed with water, brine, and dried over Na2SO4. Purification by column chromatography (1:1 ethyl acetate/hexanes) gave (R)-1-((2-cyano-4,5-difluorophenyl)sulfonyl)-N-((5-cyclohexylpyridin-2-yl)methyl)-N-phenylazetidine-2-carboxamide (42 mg, 41% yield over two steps) 1H NMR (300 MHz, CDCl3) δ 8.34 (d, J=2.3 Hz, 1H), 8.25 (t, J=7.1 Hz, 1H), 7.51 (dd, J=8.1, 2.3 Hz, 1H), 7.38 (dd, J=5.1, 1.9 Hz, 3H), 7.23-7.06 (m, 4H), 5.02-4.81 (m, 3H), 4.22-4.04 (m, 1H), 3.94 (td, J=8.1, 7.7, 4.6 Hz, 1H), 2.52 (s, 1H), 2.42-2.25 (m, 1H), 1.84 (p, J=12.8, 11.8 Hz, 6H), 1.51-1.32 (m, 5H). 19F NMR (282 MHz, CDCl3) δ−92.58 (dt, J=16.6, 8.3 Hz), −94.60-−94.83 (m). HRMS (ESI+) m/z 551.1931 [M+H]+100% purity by LCMS.

### Example 57

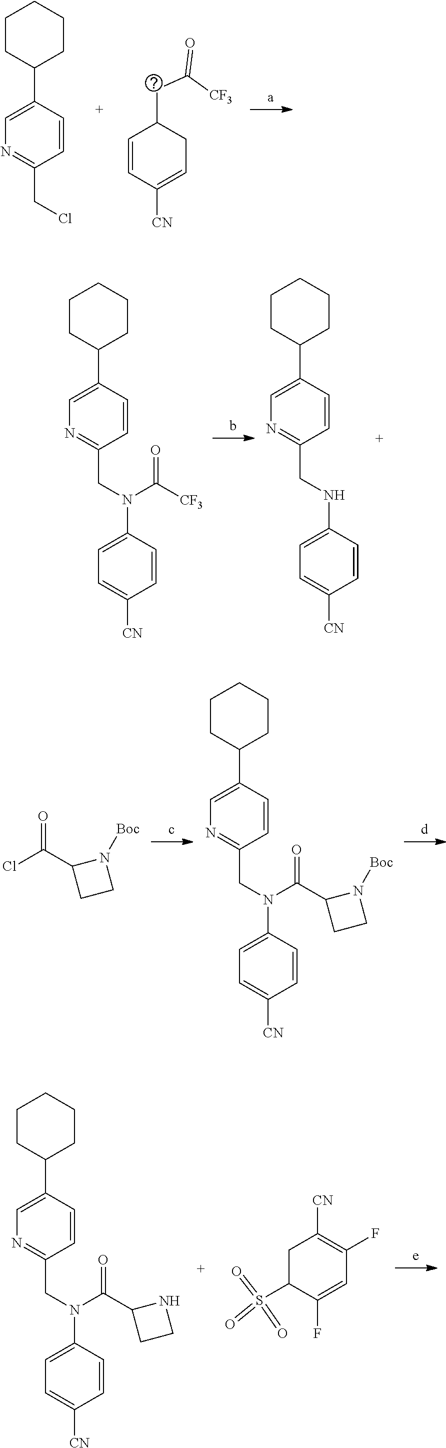

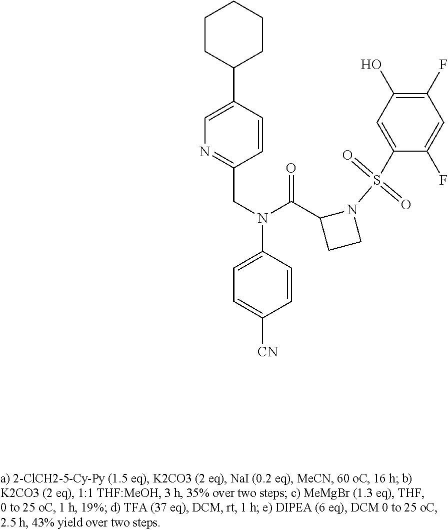 [SMILES: N#Cc1ccc(N(Cc2ccc(C3CCCCC3)cn2)C(=O)C2CCN2S(=O)(=O)c2cc(O)c(F)cc2F)cc1]

### (R)-1-((5-cyano-2,4-difluorophenyl)sulfonyl)-N-(4-cyanophenyl)-N-((5-cyclohexylpyridin-2-yl)methyl)azetidine-2-carboxamide

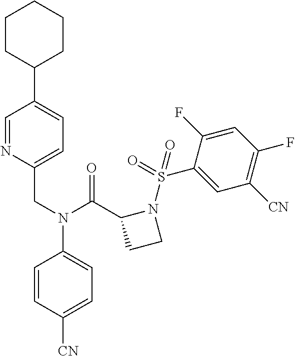 [SMILES: N#Cc1ccc(N(Cc2ccc(C3CCCCC3)cn2)C(=O)[C@H]2CCN2S(=O)(=O)c2cc(C#N)c(F)cc2F)cc1]

Step 1. To N-(4-cyanophenyl)-2,2,2-trifluoroacetamide (800 mg) under argon was added NaI (112 mg) and K2CO3 (1.03 g). The solids were then dissolved in acetonitrile (25 ml). A 1M solution of (chloromethyl)-5-cyclohexylpyridine (4.8 ml) in toluene was added to the reaction mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. Purification by column chromatography (2:8 ethyl acetate/hexanes) gave N-(4-cyanophenyl)-N-((5-cyclohexylpyridin-2-yl)methyl)-2,2,2-trifluoroacetamide as an impure mixture with starting material. 1H NMR (300 MHz, CDCl3) δ 8.39 (d, J=2.9 Hz, 1H), 7.82-7.65 (m, 2H), 7.52 (dd, J=8.0, 2.4 Hz, 1H), 7.48-7.37 (m, 2H), 7.34-7.16 (m, 1H), 5.01 (s, 2H), 2.54 (m, 1H), 1.72 (m, 6H), 1.39 (h, J=18.5, 15.9 Hz, 4H). 19F NMR (282 MHz, CDCl3) δ−66.90.

Step 2. To crude N-(4-cyanophenyl)-N-((5-cyclohexylpyridin-2-yl)methyl)-2,2,2-trifluoroacetamide (1.002 g) was added K2CO3 (714 mg) followed by THF (25 ml) and methanol (25 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave 4-(((5-cyclohexylpyridin-2-yl)methyl)amino)benzonitrile (374 mg, 35% yield over two steps). 1H NMR (300 MHz, CDCl3) δ 8.46 (dd, J=4.7, 2.3 Hz, 1H), 7.53 (dp, J=7.7, 2.3 Hz, 1H), 7.45 (dq, J=8.7, 2.3 Hz, 2H), 7.23 (dd, J=7.7, 5.0 Hz, 1H), 6.66 (dq, J=8.8, 2.3 Hz, 2H), 5.60-5.49 (m, 1H), 4.45 (t, J=4.8 Hz, 2H), 2.55 (d, J=10.7 Hz, 1H), 1.92-1.76 (m, 5H), 1.54-1.22 (m, 5H).

Step 3. To a stirred solution of 4-(((5-cyclohexylpyridin-2-yl)methyl)amino)benzonitrile in THF (10 ml) under argon at 0° C. was added a solution of 1.4M MeMgBr (1 ml) in 1:3 toluene:tetrahydrofuran. The reaction mixture was allowed to stir for fifteen minutes before tert-butyl (R)-2-(chlorocarbonyl)azetidine-1-carboxylate (653 mg) in THF (10 ml) was added. The ice bath was then removed and the reaction was allowed to reach room temperature. After two and a half hours saturated ammonium chloride solution was added to the reaction mixture. The reaction mixture was then extracted with ethyl acetate. The combined organic extracts were then washed with water, brine, and dried over Na2SO4. Purification by column chromatography (4:6 ethyl acetate/hexanes) gave tert-butyl (R)-2-((4-cyanophenyl)((5-cyclohexylpyridin-2-yl)methyl)carbamoyl)azetidine-1-carboxylate (66 mg, 9% yield). 1H NMR (300 MHz, CDCl3) δ 8.34 (d, J=4.9 Hz, 1H), 7.67 (d, J=7.6 Hz, 2H), 7.60-7.34 (m, 4H), 5.06 (s, 2H), 4.12 (dt, J=11.5, 6.9 Hz, 1H), 4.00-3.88 (m, 1H), 3.79 (t, J=7.3 Hz, 1H), 2.51 (s, 2H), 2.18 (d, J=7.5 Hz, 1H), 1.77 (d, J=12.6 Hz, 5H), 1.51-1.35 (m, 14H).

Step 4. To stirred solution of tert-butyl (R)-2-((4-cyanophenyl)((5-cyclohexylpyridin-2-yl)methyl)carbamoyl)azetidine-1-carboxylate in DCM (10 ml) under argon was added TFA (1 ml). The reaction mixture was stirred at room temperature for one hour. The reaction was then concentrated and the resulting solid ((R)—N-(4-cyanophenyl)-N-((5-cyclohexylpyridin-2-yl)methyl)azetidine-2-carboxamide TFA salt) was used directly in the next reaction.

Step 5. The solid ((R)—N-(4-cyanophenyl)-N-((5-cyclohexylpyridin-2-yl)methyl)azetidine-2-carboxamide TFA salt) from Step 4 was dissolved in DCM (5 ml) under argon, and DIPEA (0.152 ml) was added to the solution at 0° C. The reaction mixture was allowed to stir for fifteen minutes. 5-cyano-2,4-difluorobenzenesulfonyl chloride (CAS No. 1807241-08-2) (41 mg) in DCM (5 ml) was added to the reaction mixture. The ice bath was removed and the reaction was allowed to warm to room temperature. After two and a half hours saturated ammonium chloride solution was added. The reaction mixture was extracted with dichloromethane. The combined organic extracts were washed with water, brine, and dried over Na2SO4. Purification by column chromatography gave (1:1 ethyl acetate/hexanes) (R)-1-((5-cyano-2,4-difluorophenyl)sulfonyl)-N-(4-cyanophenyl)-N-((5-cyclohexylpyridin-2-yl)methyl)azetidine-2-carboxamide (34 mg, 43% yield over two steps) 1H NMR (300 MHz, CDCl3) δ 8.35 (s, 1H), 8.24 (t, J=7.1 Hz, 1H), 7.71 (d, J=8.2 Hz, 2H), 7.52 (dd, J=8.1, 2.3 Hz, 1H), 7.43-7.33 (m, 2H), 7.21-7.04 (m, 2H), 4.91 (d, J=4.8 Hz, 2H), 4.14 (q, J=7.2 Hz, 2H), 4.03-3.90 (m, 1H), 2.53 (m, 1H), 2.35 (m, 1H), 2.00 (m, 1H), 1.82 (dd, J=25.5, 10.7 Hz, 5H), 1.56-1.31 (m, 5H). 19F NMR (282 MHz, CDCl3) δ−117.12-−117.93 (m). HRMS (ESI+) m/z 576.1879[M+H]+0.100% purity by LCMS.

### Example 58

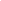

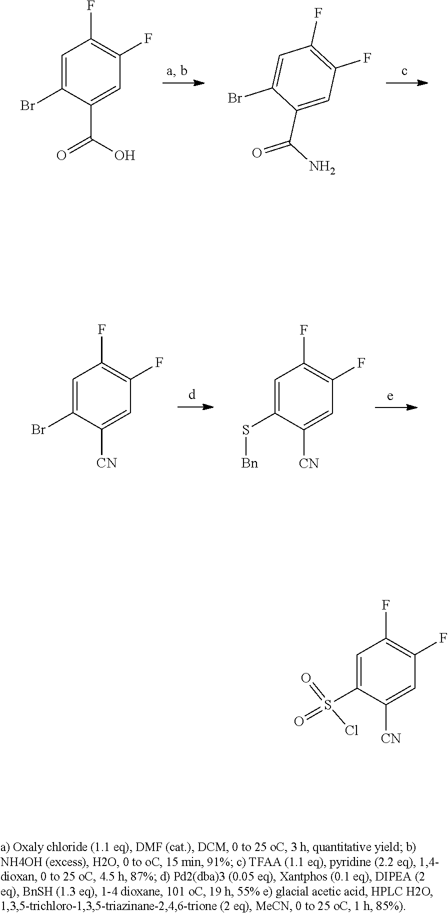 [SMILES: O=C(O)c1cc(F)c(F)cc1Br ; NC(=O)c1cc(F)c(F)cc1Br ; N#Cc1cc(F)c(F)cc1Br ; N#Cc1cc(F)c(F)cc1SCc1ccccc1 ; N#Cc1cc(F)c(F)cc1S(=O)(=O)Cl]

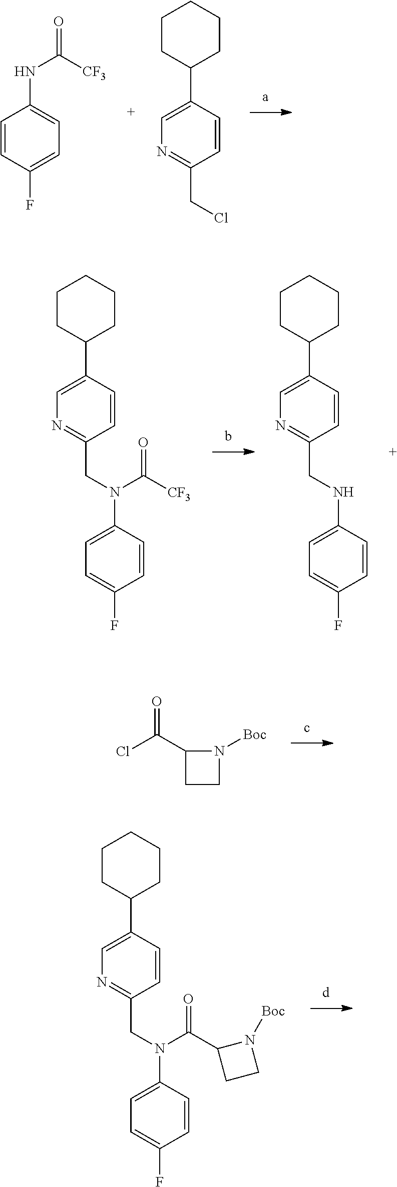 [SMILES: O=C(Nc1ccc(F)cc1)C(F)(F)F ; ClCc1ccc(C2CCCCC2)cn1 ; O=C(N(Cc1ccc(C2CCCCC2)cn1)c1ccc(F)cc1)C(F)(F)F ; Fc1ccc(NCc2ccc(C3CCCCC3)cn2)cc1 ; CC(C)(C)OC(=O)N1CCC1C(=O)Cl ; CC(C)(C)OC(=O)N1CCC1C(=O)N(Cc1ccc(C2CCCCC2)cn1)c1ccc(F)cc1]

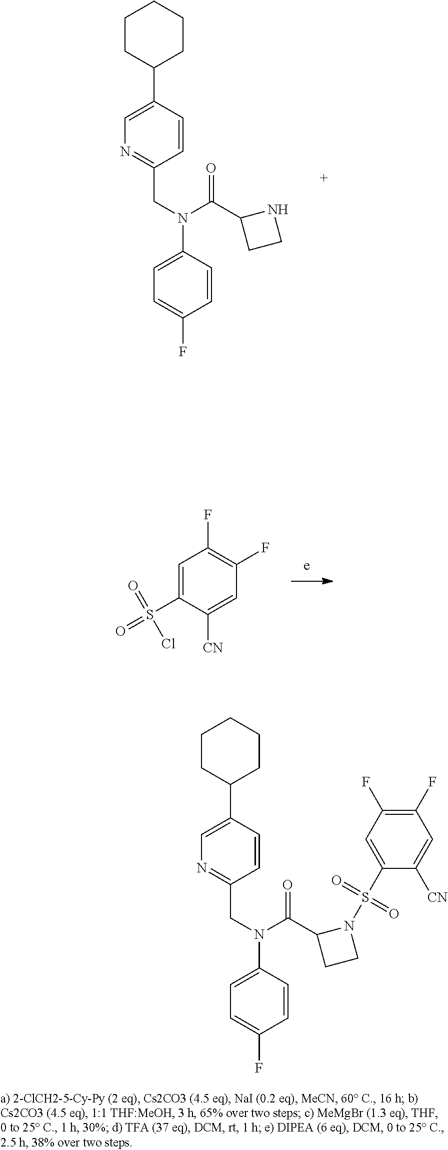 [SMILES: O=C(C1CCN1)N(Cc1ccc(C2CCCCC2)cn1)c1ccc(F)cc1 ; N#Cc1cc(F)c(F)cc1S(=O)(=O)Cl ; N#Cc1cc(F)c(F)cc1S(=O)(=O)N1CCC1C(=O)N(Cc1ccc(C2CCCCC2)cn1)c1ccc(F)cc1]

### (R)-1-((2-cyano-4,5-difluorophenyl)sulfonyl)-N-((5-cyclohexylpyridin-2-yl)methyl)-N-(4-fluorophenyl)azetidine-2-carboxamide

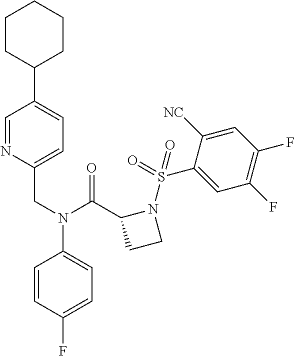 [SMILES: N#Cc1cc(F)c(F)cc1S(=O)(=O)N1CC[C@@H]1C(=O)N(Cc1ccc(C2CCCCC2)cn1)c1ccc(F)cc1]

Step 1. To a solution of 2-bromo-4,5-difluorobenzoic acid (2 g) in dichloromethane (30 ml) was added oxalyl chloride (1 ml) followed by dry DMF (1 drops) under argon. The mixture was allowed to stir at room temperature for 2.5 hours and then concentrated. Dry acetonitrile (20 ml) was added, and the solution was poured onto cold concentrated ammonium hydroxide (81 ml). The mixture was allowed to reach room temperature and then stirred for 15 minutes. Water was added to the mixture and then it was extracted with ethyl acetate. The extracts were then washed with water, brine, dried over Na2SO4 and concentrated to obtain 2-bromo-4,5-difluorobenzamide (1.802 g, 91% yield).

Step 2. To a solution of 2-bromo-4,5-difluorobenzamide (1.803 g) in dioxane (15 ml) was added anhydrous pyridine (1.20 ml). The solution was cooled in an ice-water bath, and trifluoroacetic anhydride (1.39 ml) was added. The reaction was allowed to reach room temperature and then stirred for four and a half hours. The mixture was poured onto water and extracted with ethyl acetate. The organic extracts were then washed with sodium bicarbonate. The combined organic extracts were then washed with water, brine, dried, and concentrated to obtain 2-bromo-4,5-difluorobenzonitrile (1.451 g, 87% yield).

Step 3. To 2-bromo-4,5-difluorobenzonitrile (1.15 g) was added Pd2(dba)3 (118 mg) and Xantphos (152 mg). The reaction vessel was then flushed with argon. To the solids was added dioxane (10 ml), followed by i-Pr2NEt (1.784 ml), and benzyl mercaptan (0.648 ml). The reaction mixture was allowed to stir at 101° C. for 19 hours. After cooling to room temperature, water was added, and the mixture was then extracted with ethyl acetate. The combined organic extracts were washed with water, brine, dried and concentrated. Purification by column chromatography (1:3 toluene:hexanes) gave 2-(benzylthio)-4,5-difluorobenzonitrile (737 mg, 55% Yield).

Step 4. To a solution of 2-(benzylthio)-4,5-difluorobenzonitrile (300 mg) in HPLC acetonitrile (5 ml) was added acetic acid (0.3 ml) and HPLC water (0.14 ml). The mixture was cooled to 0° C. and 1,3,5-trichloro-1,3,5-triazinane-2,4,6-trione was added (520 mg). The ice bath was removed and the reaction was stirred for one hour. Added water to the reaction and extracted with ethyl acetate. The organic extracts were washed with pH 7 buffer, water, brine, dried, and concentrated. Purification by column chromatography (96:4 hexanes:ethyl acetate) gave 2-cyano-4,5-difluorobenzenesulfonyl chloride (231 mg, 85% yield). 1H NMR (500 MHz, CDCl3) δ 8.15-8.09 (m, 1H), 7.86 (ddd, J=9.0, 6.6, 2.0 Hz, 1H). 19F NMR (471 MHz, CDCl3) δ−121.12 (dt, J=20.7, 7.6 Hz), −121.25 (dt, J=20.7, 7.7 Hz).

Step 5. To 2,2,2-trifluoro-N-(4-fluorophenyl)acetamide (500 mg) under argon was added NaI (72 mg) and Cs2CO3 (3.67 g). The solids were then dissolved in acetonitrile (20 ml). A 1M solution of 2-(chloromethyl)-5-cyclohexylpyridine (3.1 ml) in toluene was added to the reaction mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. Purification by column chromatography (2:8 ethyl acetate/hexanes) gave N-((5-cyclohexylpyridin-2-yl)methyl)-2,2,2-trifluoro-N-(4-fluorophenyl)acetamide (508 mg, 55% yield) 1H NMR (300 MHz, CDCl3) δ 8.39 (d, J=2.6 Hz, 1H), 7.72-7.41 (m, 2H), 7.28-6.90 (m, 4H), 4.99 (s, 2H), 2.54 (s, 1H), 1.95-1.19 (m, 10H).

Step 6. To N-((5-cyclohexylpyridin-2-yl)methyl)-2,2,2-trifluoro-N-(4-fluorophenyl)acetamide (508 mg) under argon was added K2CO3 (544 mg) followed by THF (10 ml) and methanol (10 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave N-((5-cyclohexylpyridin-2-yl)methyl)-4-fluoroaniline (0.443 g, 65% yield over two steps) as a green oil. 1H NMR (300 MHz, CDCl3) δ 8.45 (d, J=2.3 Hz, 1H), 7.55 (dd, J=8.0, 2.3 Hz, 1H), 7.30 (d, J=7.9 Hz, 1H), 6.96-6.82 (m, 2H), 6.69-6.54 (m, 2H), 4.42 (s, 2H), 4.15 (s, 1H), 2.63-2.46 (m, 1H), 1.96-1.74 (m, 5H), 1.49-1.22 (m, 5H).

Step 7. To a stirred solution of N-((5-cyclohexylpyridin-2-yl)methyl)-4-fluoroaniline (220 mg) in THF (6 ml) at 0° C. under argon was added a solution of 1.4M MeMgBr (1.45 ml) in 1:3 toluene:tetrahydrofuran. The reaction mixture was allowed to stir for fifteen minutes before tert-butyl (R)-2-(chlorocarbonyl)azetidine-1-carboxylate (344 mg) in THF (6 ml) was added. The ice bath was then removed and the reaction was allowed to reach room temperature. After two and a half hours saturated ammonium chloride solution was added to the reaction mixture. The reaction mixture was then extracted with ethyl acetate. The combined organic extracts were then washed with water, brine, and dried over Na2SO4. Purification by column chromatography (4:6 ethyl acetate/hexanes) gave tert-butyl (R)-2-(((5-cyclohexylpyridin-2-yl)methyl)(4-fluorophenyl)carbamoyl)azetidine-1-carboxylat e (220 mg, 30% yield). 1H NMR (300 MHz, CDCl3) δ 8.36 (dd, J=32.9, 2.2 Hz, 1H), 7.80-6.85 (m, 6H), 5.14-4.84 (m, 2H), 4.62-4.41 (m, 1H), 4.15-3.99 (m, 1H), 3.83-3.68 (m, 1H), 3.67-3.54 (m, 1H), 2.64-2.43 (m, 1H), 2.23-2.06 (m, 1H), 1.96-1.65 (m, 6H), 1.60-1.08 (m, 14H). 19F NMR (282 MHz, CDCl3) δ−112.92.

Step 8. To a solution of tert-butyl (R)-2-(((5-cyclohexylpyridin-2-yl)methyl)(4-fluorophenyl)carbamoyl)azetidine-1-carboxylat e (220 mg) in DCM (10 ml) under argon was added TFA (1 ml). The reaction mixture was stirred at room temperature for one hour. The reaction was then concentrated and the resulting solid ((R)—N-((5-cyclohexylpyridin-2-yl)methyl)-N-(4-fluorophenyl)azetidine-2-carboxamide TFA salt) was used directly in the next reaction.

Step 9. The solid ((R)—N-((5-cyclohexylpyridin-2-yl)methyl)-N-(4-fluorophenyl)azetidine-2-carboxamide TFA salt) from Step 4 was dissolved in DCM (5 ml), and DIPEA (0.5 ml) was added to the solution at 0° C. The reaction mixture was allowed to stir for fifteen minutes. 2-cyano-4,5-difluorobenzenesulfonyl chloride (145 mg) in DCM (5 ml) was added to the reaction mixture. The ice bath was removed and the reaction was allowed to warm to room temperature. After two and a half hours saturated ammonium chloride solution was added. The reaction mixture was extracted with dichloromethane. The combined organic extracts were washed with water, brine, and dried over Na2SO4. Purification by column chromatography (6:4 ethyl acetate:hexanes) gave (R)-1-((2-cyano-4,5-difluorophenyl)sulfonyl)-N-((5-cyclohexylpyridin-2-yl)methyl)-N-(4-fluorophenyl)azetidine-2-carboxamide (100 mg, 38% yield over two steps). 1H NMR (300 MHz, CDCl3) δ 8.34 (d, J=2.2 Hz, 1H), 7.97 (ddd, J=9.4, 7.4, 1.4 Hz, 1H), 7.71-7.63 (m, 1H), 7.53 (dd, J=8.1, 2.3 Hz, 1H), 7.25-7.14 (m, 3H), 7.11-7.02 (m, 2H), 5.08-4.98 (m, 1H), 4.96-4.83 (m, 2H), 4.25-4.09 (m, 2H), 3.99 (ddd, J=8.9, 7.2, 4.3 Hz, 1H), 2.52 (s, 1H), 2.41-2.26 (m, 1H), 1.93-1.74 (m, 6H), 1.49-1.31 (m, 5H). 19F NMR (282 MHz, CDCl3) δ −111.46, −124.57 (ddd, J=21.1, 9.4, 6.9 Hz), −127.85 (ddd, J=21.1, 9.0, 7.4 Hz). HRMS (ESI+) m/z 569.1839 [M+H]+0.100% purity by LCMS.

### Example 59

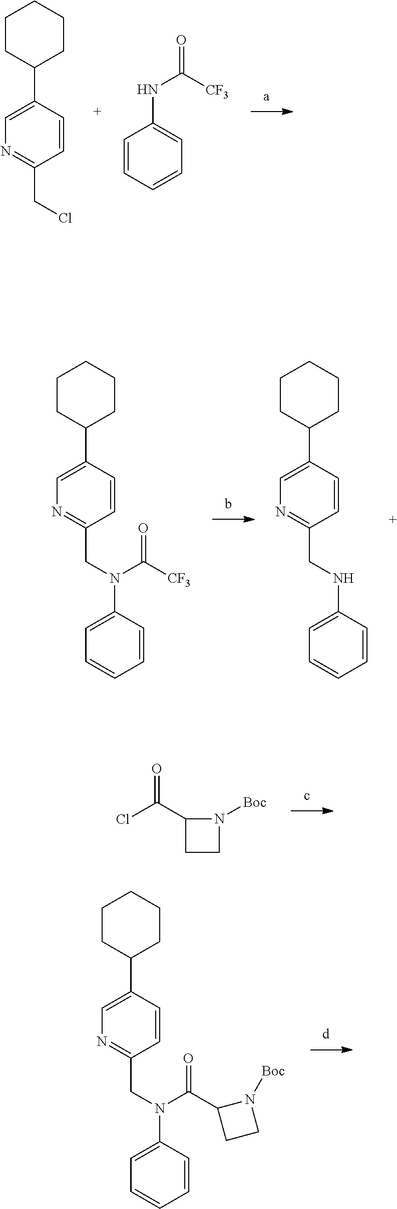 [SMILES: ClCc1ccc(C2CCCCC2)cn1 ; O=C(Nc1ccccc1)C(F)(F)F ; O=C(N(Cc1ccc(C2CCCCC2)cn1)c1ccccc1)C(F)(F)F ; c1ccc(NCc2ccc(C3CCCCC3)cn2)cc1 ; CC(C)(C)OC(=O)N1CCC1C(=O)Cl ; CC(C)(C)OC(=O)N1CCC1C(=O)N(Cc1ccc(C2CCCCC2)cn1)c1ccccc1]

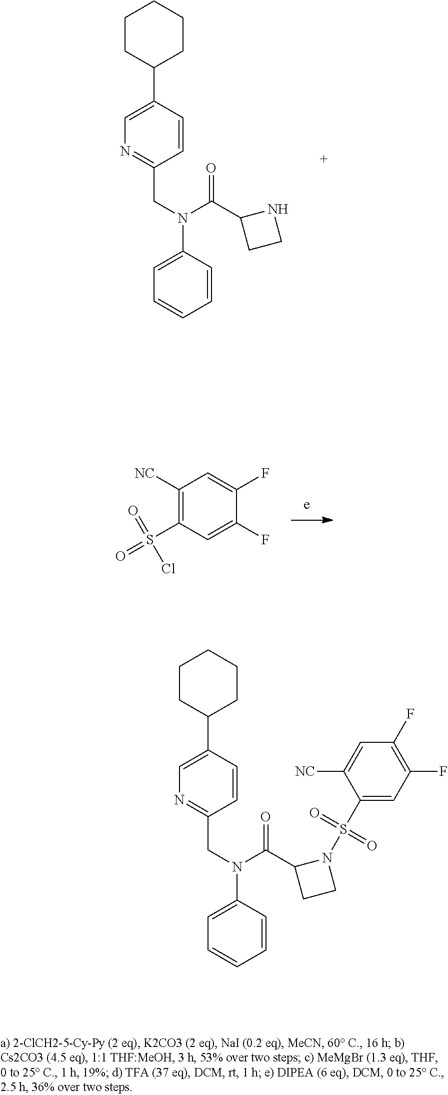 [SMILES: O=C(C1CCN1)N(Cc1ccc(C2CCCCC2)cn1)c1ccccc1 ; N#Cc1cc(F)c(F)cc1S(=O)(=O)Cl ; N#Cc1cc(F)c(F)cc1S(=O)(=O)N1CCC1C(=O)N(Cc1ccc(C2CCCCC2)cn1)c1ccccc1]

### (R)-1-((2-cyano-4,5-difluorophenyl)sulfonyl)-N-((5-cyclohexylpyridin-2-yl)methyl)-N-phenylazetidine-2-carboxamide

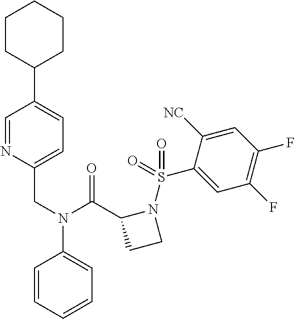 [SMILES: N#Cc1cc(F)c(F)cc1S(=O)(=O)N1CC[C@@H]1C(=O)N(Cc1ccc(C2CCCCC2)cn1)c1ccccc1]

Step 1. To 2,2,2-trifluoro-N-phenylacetamide (460 mg) under argon was added NaI (99 mg) and Cs2CO3 (3.5 g). The solids were then dissolved in acetonitrile (30 ml). A 1M solution of (chloromethyl)-5-cyclohexylpyridine (3.4 mL) in toluene was added to the reaction mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. Purification by column chromatography (2:8 ethyl acetate/hexanes) gave N-((5-cyclohexylpyridin-2-yl)methyl)-2,2,2-trifluoro-N-phenylacetamide (401 mg, 49% yield).

Step 2. To N-((5-cyclohexylpyridin-2-yl)methyl)-2,2,2-trifluoro-N-phenylacetamide (420 mg) under argon was added K2CO3 (326 mg) followed by THF (8 ml) and methanol (8 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave N-((5-cyclohexylpyridin-2-yl)methyl)aniline (0.333 g, 53% yield over two steps) as a green oil. 1H NMR (300 MHz, CDCl3) δ 8.45 (d, J=2.3 Hz, 1H), 7.50 (dd, J=8.0, 2.3 Hz, 1H), 7.28 (t, J=1.6 Hz, 1H), 7.25-7.16 (m, 2H), 6.78-6.66 (m, 2H), 4.75 (s, 1H), 4.44 (d, J=4.7 Hz, 2H), 2.65-2.45 (m, 1H), 1.96-1.66 (m, 5H), 1.55-1.18 (m, 5H).

Step 3. To a stirred solution of N-((5-cyclohexylpyridin-2-yl)methyl)aniline in THF (8 ml) at 0° C. under argon was added a solution of 1.4M MeMgBr (1.32 ml) in 1:3 toluene:tetrahydrofuran. The reaction mixture was allowed to stir for fifteen minutes before tert-butyl (R)-2-(chlorocarbonyl)azetidine-1-carboxylate (0.544 g) in THF (8 ml) was added. The ice bath was then removed and the reaction was allowed to reach room temperature. After two and a half hours saturated ammonium chloride solution was added to the reaction mixture. The reaction mixture was then extracted with ethyl acetate. The combined organic extracts were then washed with water, brine, and dried over Na2SO4. Purification by column chromatography (4:6 ethyl acetate/hexanes) gave tert-butyl (R)-2-(((5-cyclohexylpyridin-2-yl)methyl)(phenyl)carbamoyl)azetidine-1-carboxylate (102 mg, 19% yield). 1H NMR (300 MHz, CDCl3) δ 8.33 (d, J=2.1 Hz, 1H), 7.56-7.42 (m, 2H), 7.41-7.28 (m, 3H), 7.15 (d, J=14.8 Hz, 2H), 5.09 (d, J=15.0 Hz, 2H), 4.58 (s, 1H), 4.08 (td, J=8.8, 7.0 Hz, 1H), 3.75 (td, J=8.4, 5.7 Hz, 1H), 2.50 (s, 1H), 2.29-2.07 (m, 2H), 1.97-1.70 (m, 5H), 1.52-1.20 (m, 14H).

Step 4. To a solution of tert-butyl (R)-2-(((5-cyclohexylpyridin-2-yl)methyl)(phenyl)carbamoyl)azetidine-1-carboxylate (50 mg) in DCM (10 ml) under argon was added TFA (1 ml). The reaction mixture was stirred at room temperature for one hour. The reaction was then concentrated and the resulting solid ((R)—N-((5-cyclohexylpyridin-2-yl)methyl)-N-phenylazetidine-2-carboxamide TFA salt) was used directly in the next reaction.

Step 5. The solid ((R)—N-((5-cyclohexylpyridin-2-yl)methyl)-N-phenylazetidine-2-carboxamide TFA salt) was dissolved in DCM (5 ml), and DIPEA (0.1 ml) was added to the solution at 0° C. The reaction mixture was allowed to stir for fifteen minutes. 2-cyano-4,5-difluorobenzenesulfonyl chloride (28.4 mg) in DCM (5 ml) was added to the reaction mixture. The ice bath was removed and the reaction was allowed to warm to room temperature. After two and a half hours saturated ammonium chloride solution was added. The reaction mixture was extracted with dichloromethane. The combined organic extracts were washed with water, brine, and dried over Na2SO4. Purification by column chromatography (1:1 ethyl acetate:hexanes) gave (R)-1-((2-cyano-4,5-difluorophenyl)sulfonyl)-N-((5-cyclohexylpyridin-2-yl)methyl)-N-phenylazetidine-2-carboxamide (22 mg, 36% yield over two steps). 1H NMR (500 MHz, CDCl3) δ 8.35 (d, J=2.2 Hz, 1H), 7.96 (dd, J=9.3, 7.3 Hz, 1H), 7.71-7.61 (m, 2H), 7.44-7.33 (m, 4H), 7.18 (d, J=7.2 Hz, 2H), 5.19-4.93 (m, 3H), 4.17 (q, J=7.9 Hz, 1H), 4.09-3.91 (m, 1H), 2.55 (s, 1H), 2.39-2.29 (m, 1H), 1.93-1.77 (m, 6H), 1.49-1.34 (m, 5H). 19F NMR (471 MHz, CDCl3) δ−124.50 (m), −127.80 (m). HRMS (ESI+) m/z 551.1963 [M+H]+100% purity by LCMS.

### Example 60

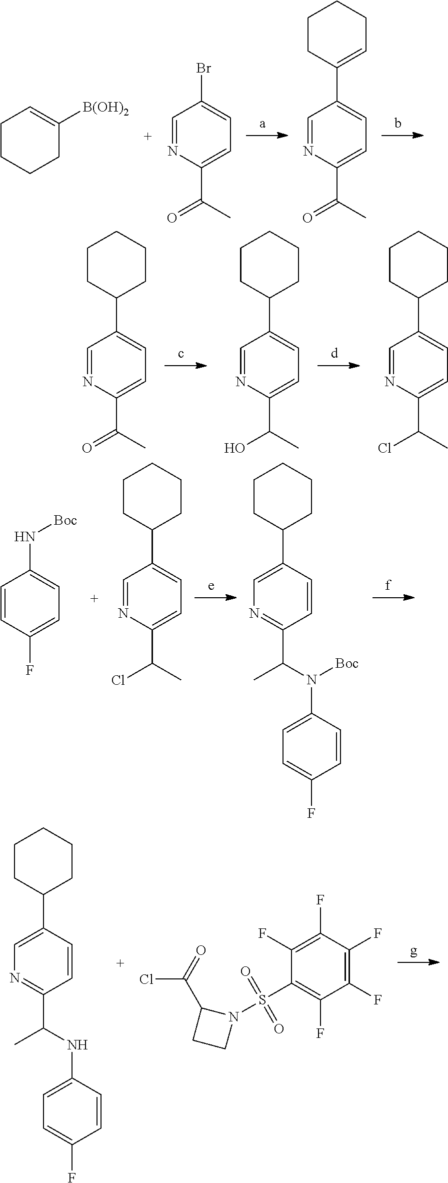

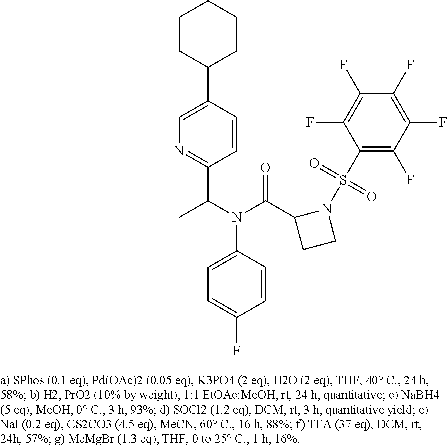 [SMILES: CC(c1ccc(C2CCCCC2)cn1)N(C(=O)C1CCN1S(=O)(=O)c1c(F)c(F)c(F)c(F)c1F)c1ccc(F)cc1]

### (2R)—N-(1-(5-cyclohexylpyridin-2-yl)ethyl)-N-(4-fluorophenyl)-1-((perfluorophenyl)sulfonyl)azetidine-2-carboxamide

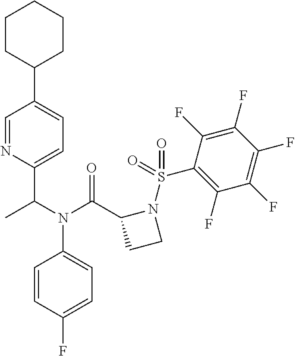 [SMILES: CC(c1ccc(C2CCCCC2)cn1)N(C(=O)[C@H]1CCN1S(=O)(=O)c1c(F)c(F)c(F)c(F)c1F)c1ccc(F)cc1]

Step 1. To 1-(5-bromopyridin-2-yl)ethan-1-one (650 mg) under argon was added cyclohex-1-en-1-ylboronic acid (756 mg) followed by potassium phosphate tribasic (1.272 g). The solids were then dissolved in THF (20 mL) followed by the addition of SPhos (123 mg) and Pd(OAc)2 (33 mg). The resulting mixture was heated with stirring to 40° C. for twenty four hours. The reaction mixture was then allowed to cool to room temperature. Water was added and the mixture was then extracted with ethyl acetate. The combined organic extracts were then washed with water, brine, and dried over Na2SO4. Purification via column chromatography (2:8 ethyl acetate/hexanes) gave 1-(5-(cyclohex-1-en-1-yl)pyridin-2-yl)ethan-1-one (380 mg, 58% yield). 1H NMR (300 MHz, CDCl3) δ 8.75 (d, J=2.3 Hz, 1H), 8.04 (dt, J=8.3, 0.6 Hz, 1H), 7.89-7.83 (m, 1H), 6.39 (tt, J=4.0, 1.7 Hz, 1H), 2.79 (d, J=0.5 Hz, 3H), 2.44 (dtd, J=7.9, 4.2, 2.2 Hz, 2H), 2.34-2.25 (m, 2H), 1.89-1.79 (m, 2H), 1.77-1.66 (m, 2H).

Step 2. To 1-(5-(cyclohex-1-en-1-yl)pyridin-2-yl)ethan-1-one (380 mg) was added Pt(IV)02 (40 mg), followed by EtOAc (10 ml) and methanol (10 ml). The round bottom was evacuated and charged with H2. The reaction was allowed to stir at room temperature for twenty-four hours. Ethyl acetate was added to the reaction mixture, and then filtered over celite. The celite was then washed with ethyl acetate. This gave 1-(5-cyclohexylpyridin-2-yl)ethan-1-one quantitatively.

Step 3. To 1-(5-cyclohexylpyridin-2-yl)ethan-1-one (390 mg) in MeOH (10 ml) under argon was added NaBH4 (363 mg) at 0° C. The reaction was allowed to warm to room temperature and stirred for three hours. Water was added to the reaction. The reaction mixture was extracted with ethyl acetate. The combined organic extracts were washed with water, brine, and dried over Na2SO4. Purification by column chromatography gave 1-(5-cyclohexylpyridin-2-yl)ethan-1-ol (366 mg, 93% yield). 1H NMR (300 MHz, CDCl3) δ 8.48 (s, 1H), 7.82 (d, J=8.2 Hz, 1H), 7.46 (d, J=8.1 Hz, 1H), 5.11 (q, J=6.7 Hz, 1H), 2.63 (s, 1H), 1.97-1.76 (m, 6H), 1.63-1.56 (m, 3H), 1.51-1.38 (m, 4H).

Step 4. To 1-(5-cyclohexylpyridin-2-yl)ethan-1-ol (358 mg) under argon was added DCM (10 ml) followed by thionyl chloride (0.13 ml). The reaction was allowed to stir at room temperature for three hours. Dichloromethane was added to the reaction mixture and washed with aqueous sodium bicarbonate. The organic layer was then washed with water, brine, and dried over Na2SO4. The organic layer was then concentrated under vacuum to give 2-(1-chloroethyl)-5-cyclohexylpyridine (quantitative yield) which was stored as a 1M solution in toluene under Ar. No data was collected due to the instability of the product.

Step 5. To tert-butyl (4-fluorophenyl)carbamate (277 mg) under argon was added NaI (40 mg) and Cs2CO3 (1.943 g). The solids were then dissolved in acetonitrile (13 ml). A 1M solution of 2-(1-chloroethyl)-5-cyclohexylpyridine (1.724 ml) in toluene was added to the reaction mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. Purification by column chromatography (2:8 ethyl acetate/hexanes) gave tert-butyl (1-(5-cyclohexylpyridin-2-yl)ethyl)(4-fluorophenyl)carbamate (449 mg, 88% yield) 1H NMR (300 MHz, CDCl3) δ 8.44 (dd, J=2.3, 1.2 Hz, 1H), 7.61 (dt, J=8.2, 2.0 Hz, 1H), 7.51-7.40 (m, 1H), 7.37-7.29 (m, 2H), 7.11-6.90 (m, 2H), 6.46 (s, 1H), 5.29-5.15 (m, 1H), 2.57 (s, 1H), 1.94-1.71 (m, 8H), 1.59-1.50 (m, 9H), 1.48-1.32 (m, 5H). 19F NMR (282 MHz, CDCl3) δ−120.16.

Step 6. To tert-butyl (1-(5-cyclohexylpyridin-2-yl)ethyl)(4-fluorophenyl)carbamate (60 mg) under argon in DCM (10 ml) was added TFA (0.6 ml). The reaction was quenched with aqueous ammonium chloride and the reaction mixture was extracted with dichloromethane. The combined organic extracts were then washed with water, brine, and dried over Na2SO4. Purification by column chromatography (2:8 ethyl acetate/hexanes) gave N-(1-(5-cyclohexylpyridin-2-yl)ethyl)-4-fluoroaniline (25 mg, 57% yield).

Step 7. To a solution of N-(1-(5-cyclohexylpyridin-2-yl)ethyl)-4-fluoroaniline (25 mg) in THF (3 ml) at 0° C. under argon was added a solution of 1.4M MeMgBr (0.08 ml) in 1:3 toluene:tetrahydrofuran. The reaction mixture was allowed to stir for fifteen minutes. (R)-1-((perfluorophenyl)sulfonyl)azetidine-2-carbonyl chloride (58 mg) in THF (3 ml) was added to the reaction mixture. The ice bath was removed and the reaction was allowed to warm to room temperature. After two and a half hours saturated ammonium chloride solution was added. The reaction mixture was extracted with ethyl acetate. The combined organic extracts were washed with water, brine, and dried over Na2SO4. Purification by column chromatography (1:1 ethyl acetate/hexanes) followed by purification by HPLC 1.8 ml/min flow rate 80:20 acetonitrile/water on a phenomenal column Luna 5u PFP 100A 250×10 mm with a PDA detector gave (2R)—N-(1-(5-cyclohexylpyridin-2-yl)ethyl)-N-(4-fluorophenyl)-1-((perfluorophenyl)sulfonyl) azetidine-2-carboxamide (3 mg, 6% yield). 1H NMR (300 MHz, CDCl3) δ 8.33 (dd, J=15.7, 2.3 Hz, 1H), 7.52 (dd, J=8.1, 2.3 Hz, 1H), 7.34-6.54 (m, 4H), 5.91 (p, J=7.1 Hz, 1H), 4.73 (dd, J=9.0, 7.0 Hz, 1H), 4.13 (dt, J=8.9, 7.1 Hz, 1H), 4.08-3.98 (m, 1H), 2.53 (s, 1H), 2.28 (ddt, J=10.9, 9.1, 7.1 Hz, 1H), 2.02-1.63 (m, 6H), 1.55-1.12 (m, 8H). 19F NMR (282 MHz, CDCl3) δ−111.50 (p, J=6.4 Hz), −135.72-−135.95 (m), −136.52 (d, J=8.1 Hz), −147.03 (tt, J=21.3, 6.4 Hz), −159.56-−159.85 (m). HRMS (ESI+) m/z 612.175 [M+H]+. 100% purity.

### Example 61

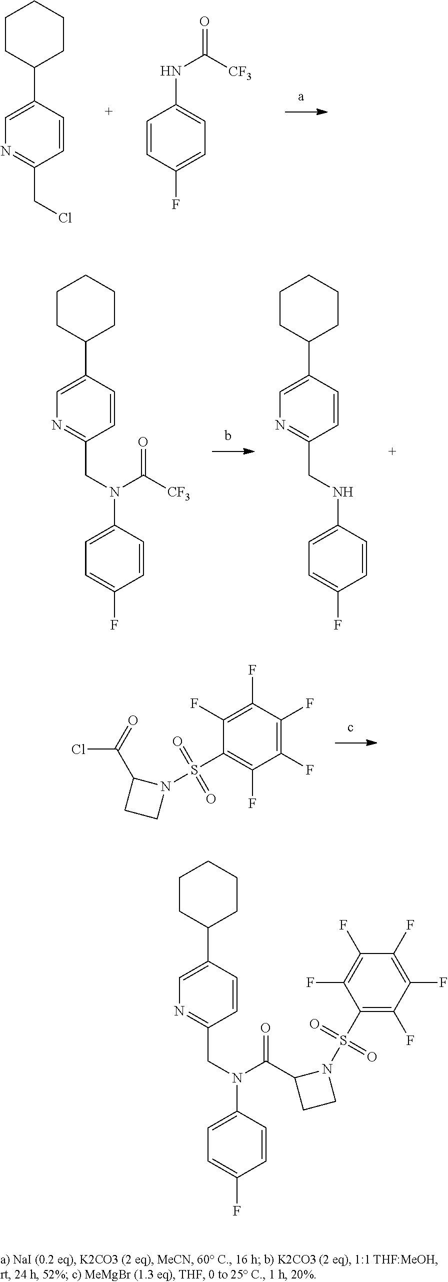 [SMILES: ClCc1ccc(C2CCCCC2)cn1 ; O=C(Nc1ccc(F)cc1)C(F)(F)F ; O=C(N(Cc1ccc(C2CCCCC2)cn1)c1ccc(F)cc1)C(F)(F)F ; Fc1ccc(NCc2ccc(C3CCCCC3)cn2)cc1 ; O=C(Cl)C1CCN1S(=O)(=O)c1c(F)c(F)c(F)c(F)c1F ; O=C(C1CCN1S(=O)(=O)c1c(F)c(F)c(F)c(F)c1F)N(Cc1ccc(C2CCCCC2)cn1)c1ccc(F)cc1]

### (R)—N-((5-cyclohexylpyridin-2-yl)methyl)-N-(4-fluorophenyl)-1-((perfluorophenyl)sulfonyl)azetidine-2-carboxamide

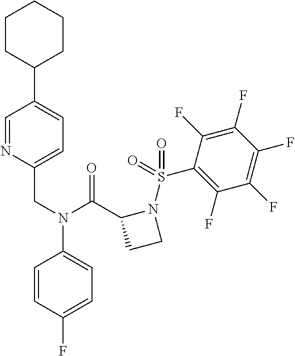 [SMILES: O=C([C@H]1CCN1S(=O)(=O)c1c(F)c(F)c(F)c(F)c1F)N(Cc1ccc(C2CCCCC2)cn1)c1ccc(F)cc1]

Step 1. To 2,2,2-trifluoro-N-(4-fluorophenyl)acetamide (0.724 g) was added NaI (mg) and Cs2CO3 (4.445 g). The solids were then dissolved in acetonitrile (40 ml). A 1M solution of 2-(1-chloroethyl)-5-cyclohexylpyridine (1.79 ml) in toluene was added to the reaction mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and dried over Na2SO4. Purification by column chromatography (2:8 ethyl acetate/hexanes) gave N-(1-(5-cyclohexylpyridin-2-yl)ethyl)-2,2,2-trifluoro-N-(4-fluorophenyl)acetamide. 1H NMR (300 MHz, CDCl3) δ 8.39 (d, J=2.6 Hz, 1H), 7.72-7.41 (m, 2H), 7.28-6.90 (m, 4H), 4.99 (s, 2H), 2.54 (s, 1H), 1.95-1.19 (m, 10H).

Step 2. To N-(1-(5-cyclohexylpyridin-2-yl)ethyl)-2,2,2-trifluoro-N-(4-fluorophenyl)acetamide (679 mg) was added K2CO3 (0.504 g) followed by THF (15 ml) and methanol (15 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave N-(1-(5-cyclohexylpyridin-2-yl)ethyl)-4-fluoroaniline (517 mg, 52% yield over two steps) as a green oil. 1H NMR (300 MHz, CDCl3) δ 8.45 (d, J=2.3 Hz, 1H), 7.55 (dd, J=8.0, 2.3 Hz, 1H), 7.30 (d, J=7.9 Hz, 1H), 6.96-6.82 (m, 2H), 6.69-6.54 (m, 2H), 4.42 (s, 2H), 4.15 (s, 1H), 2.63-2.46 (m, 1H), 1.96-1.74 (m, 5H), 1.49-1.22 (m, 5H).

Step 3. To a solution of N-(1-(5-cyclohexylpyridin-2-yl)ethyl)-4-fluoroaniline (88 mg) in THF (5 ml) at 0° C. under argon was added a solution of 1.4M MeMgBr (0.557 ml) in 1:3 toluene:tetrahydrofuran. The reaction mixture was allowed to stir for fifteen minutes. (R)-1-((perfluorophenyl)sulfonyl)azetidine-2-carbonyl chloride (141 mg) in THF (5 ml) was added to the reaction mixture. The ice bath was removed and the reaction was allowed to warm to room temperature. After two and a half hours saturated ammonium chloride solution was added. The reaction mixture was extracted with ethyl acetate. The combined organic extracts were washed with water, brine, and dried over Na2SO4. Purification by column chromatography (1:1 ethyl acetate/hexanes) gave (2R)—N-(1-(5-cyclohexylpyridin-2-yl)ethyl)-N-(4-fluorophenyl)-1-((perfluorophenyl)sulfonyl) azetidine-2-carboxamide (36 mg, 20% yield) 1H NMR (300 MHz, CDCl3) δ 8.35 (d, J=2.3 Hz, 1H), 7.57 (dd, J=8.1, 2.3 Hz, 1H), 7.34-6.99 (m, 5H), 5.14-4.69 (m, 3H), 4.23-3.96 (m, 2H), 2.53 (s, 1H), 2.32 (ddt, J=10.7, 9.0, 7.0 Hz, 1H), 2.06-1.76 (m, 6H), 1.57-1.21 (m, 5H). 19F NMR (282 MHz, CDCl3) δ−111.44, −135.61-−136.46 (m), −146.85 (t, J=21.0 Hz), −159.56 (tt, J=20.8, 6.0 Hz). HRMS (ESI+) m/z 598.156 [M+H]+. LCMS 99% purity.

### Example 62

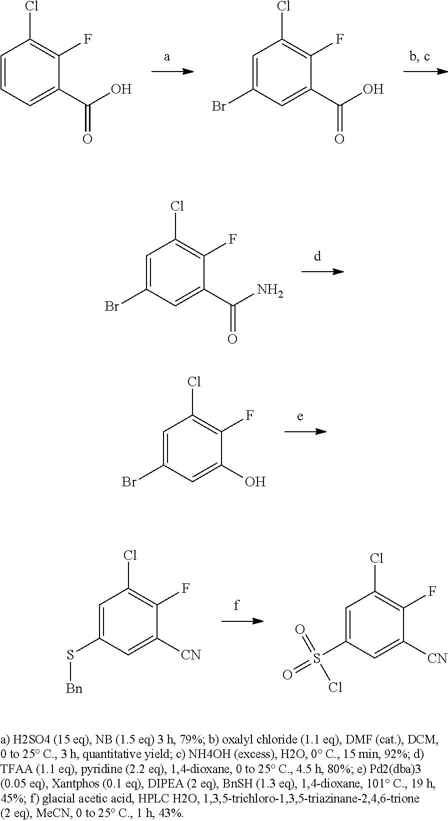 [SMILES: O=C(O)c1cccc(Cl)c1F ; O=C(O)c1cc(Br)cc(Cl)c1F ; NC(=O)c1cc(Br)cc(Cl)c1F ; Oc1cc(Br)cc(Cl)c1F ; N#Cc1cc(SCc2ccccc2)cc(Cl)c1F ; N#Cc1cc(S(=O)(=O)Cl)cc(Cl)c1F]

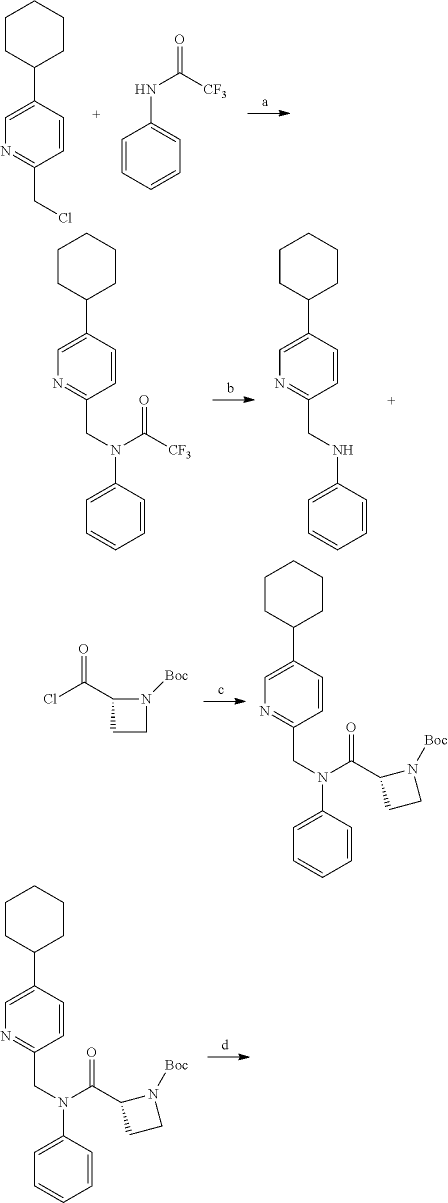

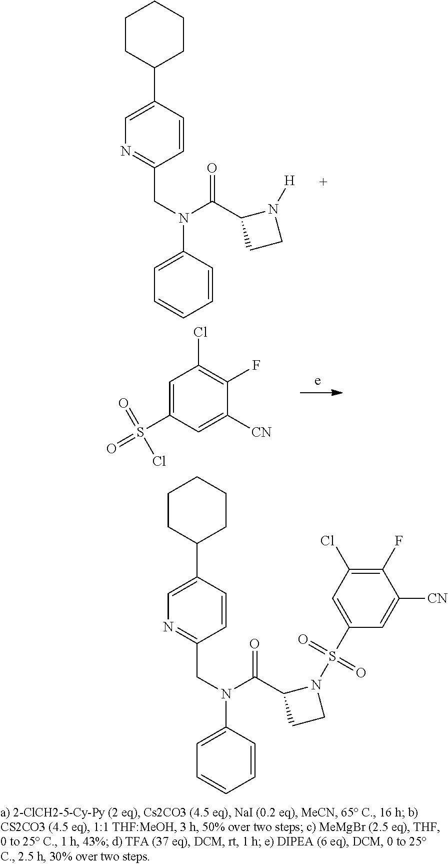

### (R)-1-((3-chloro-5-cyano-4-fluorophenyl)sulfonyl)-N-((5-cyclohexylpyridin-2-yl)methyl)-N-phenylazetidine-2-carboxamide

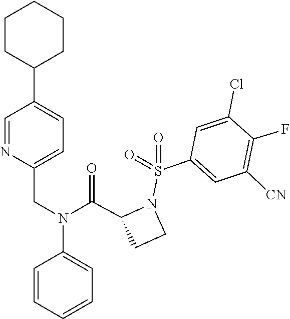 [SMILES: N#Cc1cc(S(=O)(=O)N2CC[C@@H]2C(=O)N(Cc2ccc(C3CCCCC3)cn2)c2ccccc2)cc(Cl)c1F]

Step 1. To 2-fluoro-3-chlorobenzoic acid (1.5 g) was added concentrated H2504 (7 ml) and N-bromosuccinimide (1.6 g). The reaction mixture was heated with stirring at 60° C. for three hours under argon. Reaction was then allowed to cool to room temperature and poured onto ice water. This mixture was allowed to stir at room temperature for five minutes, and then filtered. The solid was then washed with room temperature water. The solid was then dissolved in ethyl acetate and extracted with 3M sodium hydroxide. The ethyl acetate layer was then discarded, and the aqueous layer was then acidified with 3M HCl. The aqueous layer was extracted with ethyl acetate. The combined extracts were washed with water, brine, dried over Na2SO4 and concentrated to obtain 5-bromo-2-fluoro-3-chlorobenzoic acid (1.707 g, 79% yield). This product was obtained as a somewhat impure mixture resulting from non-complete regio-selectivity of the bromination. These isomers were separated at a later stage.

Step 2. To a solution of 5-bromo-2-fluoro-3-chlorobenzoic acid (3.165 g) in dichloromethane (40 ml) was added oxalyl chloride (1.6 ml) followed by dry DMF (5 drops) under argon. The mixture was allowed to stir at room temperature for 2.5 hours and then concentrated. Dry acetonitrile (30 ml) was added, and the solution was poured onto cold concentrated ammonium hydroxide (150 ml). The mixture was allowed to reach room temperature and then stirred for 15 minutes. Water was added to the mixture and then it was extracted with ethyl acetate. The extracts were then washed with water, brine, dried over Na2SO4 and concentrated to obtain crude 5-bromo-2-fluoro-3-chlorobenzamide (2.89 g, 92% yield).

Step 3. To a solution of 5-bromo-2-fluoro-3-chlorobenzamide (3.050 g) in dioxane (40 ml) was added anhydrous pyridine (1.962 ml). The solution was cooled on an ice-water bath, and trifluoroacetic anhydride (1.868 ml) was added. The reaction was allowed to reach room temperature and then stirred for four and a half hours. The mixture was poured onto water and extracted with ethyl acetate. The organic extracts were then washed with sodium bicarbonate. The combined organic extracts were then washed with water, brine, dried, and concentrated to obtain crude 5-bromo-2-fluoro-3-chlorobenzonitrile (2.258 g, 80% yield).

Step 4. To 5-bromo-2-fluoro-3-chlorobenzonitrile (640 mg) was added Pd2(dba)3 (62 mg) and Xantphos (78.9 mg). The reaction vessel was then flushed with argon. To the solids was added dioxane (10 ml), followed by i-Pr2NEt (0.935 ml), and benzyl mercaptan (0.33 ml). The reaction mixture was allowed to stir at 101° C. for 19 hours. After cooling to room temperature, water was added, and the mixture was then extracted with ethyl acetate. The combined organic extracts were washed with water, brine, dried and concentrated. Purification by column chromatography (1:3 toluene:hexanes) to resolve isomeric impurities gave pure 2-fluoro-3-chloro-5-(phenylthio)benzonitrile (340 mg, 45% Yield).

Step 5. To a solution of 2-fluoro-3-chloro-5-(phenylthio)benzonitrile (1.03 g) in HPLC acetonitrile (30 ml) was added acetic acid (0.968 ml) and HPLC water (0.484 ml). The mixture was cooled to 0° C. and 1,3,5-trichloro-1,3,5-triazinane-2,4,6-trione was added (1.726 g). The ice bath was removed and the reaction was stirred for one hour. Added water to the reaction and extracted with ethyl acetate. The organic extracts were washed with pH 7 buffer, water, brine, dried, and concentrated. Purification by column chromatography (96:4 hexanes:ethyl acetate) gave pure 3-chloro-5-cyano-4-fluorobenzenesulfonyl chloride (342 mg, 42% yield). 1H NMR (300 MHz, CDCl3) δ 8.37 (dd, J=6.1, 2.3 Hz, 1H), 8.27 (dd, J=5.0, 2.3 Hz, 1H). 19F NMR (282 MHz, CDCl3) δ−95.35 (t, J=5.6 Hz).

Step 6. To 2,2,2-trifluoro-N-phenylacetamide (631 mg) under argon was added NaI (mg) and Cs2CO3 (3.5 g). The solids were then dissolved in acetonitrile (30 ml). A 1M solution of (chloromethyl)-5-cyclohexylpyridine (ml) in toluene was added to the mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. Purification by column chromatography (2:8 ethyl acetate/hexanes) gave N-((5-cyclohexylpyridin-2-yl)methyl)-2,2,2-trifluoro-N-phenylacetamide contaminated with some SM (2,2,2-trifluoro-N-phenylacetamide) (667 mg), and taken as such to next step.

Step 7. To N-((5-cyclohexylpyridin-2-yl)methyl)-2,2,2-trifluoro-N-phenylacetamide (667 mg) under argon was added K2CO3 (517 mg) followed by THF (12 ml) and methanol (12 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave pure N-((5-cyclohexylpyridin-2-yl)methyl)aniline (440 mg, 50% yield over two steps). 1H NMR (300 MHz, CDCl3) δ 8.45 (d, J=2.3 Hz, 1H), 7.50 (dd, J=8.0, 2.3 Hz, 1H), 7.28 (t, J=1.6 Hz, 1H), 7.25-7.16 (m, 2H), 6.78-6.66 (m, 2H), 4.75 (s, 1H), 4.44 (d, J=4.7 Hz, 2H), 2.65-2.45 (m, 1H), 1.96-1.66 (m, 5H), 1.55-1.18 (m, 5H).

Step 8. To a stirred solution of N-((5-cyclohexylpyridin-2-yl)methyl)aniline (651 mg) in THF (15 ml) at 0° C. under argon was added a solution of 1.4M MeMgBr (3.11 ml) in 1:3 toluene:tetrahydrofuran. The reaction mixture was allowed to stir for fifteen minutes before tert-butyl (R)-2-(chlorocarbonyl)azetidine-1-carboxylate (709 mg) in THF (10 ml) was added. The ice bath was then removed and the reaction was allowed to reach room temperature. After two and a half hours saturated ammonium chloride solution was added. The reaction mixture was then extracted with ethyl acetate. The combined organic extracts were then washed with water, brine, and dried over Na2SO4. Purification by column chromatography (4:6 ethyl acetate/hexanes) gave tert-butyl (R)-2-(((5-cyclohexylpyridin-2-yl)methyl) (phenyl)carbamoyl)azetidine-1-carboxylate (312 mg, 43% yield). 1H NMR (300 MHz, CDCl3) δ 8.33 (d, J=2.1 Hz, 1H), 7.56-7.42 (m, 2H), 7.41-7.28 (m, 3H), 7.15 (d, J=14.8 Hz, 2H), 5.09 (d, J=15.0 Hz, 2H), 4.58 (s, 1H), 4.08 (td, J=8.8, 7.0 Hz, 1H), 3.75 (td, J=8.4, 5.7 Hz, 1H), 2.50 (s, 1H), 2.29-2.07 (m, 2H), 1.97-1.70 (m, 5H), 1.52-1.20 (m, 14H).

Step 9. To a solution of tert-butyl (R)-2-(((5-cyclohexylpyridin-2-yl)methyl)(phenyl)carbamoyl)azetidine-1-carboxylate in DCM (10 ml) under argon was added TFA (1 ml). The reaction mixture was stirred at room temperature for one hour. The reaction was then concentrated and the resulting solid ((R)—N-((5-cyclohexylpyridin-2-yl)methyl)-N-phenylazetidine-2-carboxamide TFA salt) was used directly in the next reaction.

Step 10. The solid ((R)—N-((5-cyclohexylpyridin-2-yl)methyl)-N-phenylazetidine-2-carboxamide TFA salt) from Step 4 was dissolved in DCM (10 ml), and DIPEA (0.7 ml) was added to the solution at 0° C. The reaction mixture was allowed to stir for fifteen minutes. 3-chloro-5-cyano-4-fluorobenzenesulfonyl chloride (200 mg) in DCM (10 ml) was added. The ice bath was removed and the reaction was allowed to warm to room temperature. After two and a half hours saturated ammonium chloride solution was added. The reaction mixture was extracted with dichloromethane. The combined organic extracts were washed with water, brine, and dried over Na2SO4. Purification by column chromatography (1:1 ethyl acetate/hexanes) gave (R)-1-((3-chloro-5-cyano-4-fluorophenyl)sulfonyl)-N-((5-cyclohexylpyridin-2-yl)methyl)-N-phenylazetidine-2-carboxamide (113 mg, 30% yield over two steps) 1H NMR (300 MHz, CDCl3) δ 8.41-8.28 (m, 2H), 8.18 (dd, J=5.2, 2.2 Hz, 1H), 7.56 (dd, J=8.0, 2.3 Hz, 1H), 7.40 (dd, J=5.0, 1.8 Hz, 3H), 7.35-7.30 (m, 1H), 7.23-7.11 (m, 2H), 5.10-4.92 (m, 3H), 3.96 (q, J=7.9 Hz, 1H), 3.69 (ddd, J=9.0, 7.0, 4.1 Hz, 1H), 2.53 (s, 1H), 2.45-2.29 (m, 1H), 1.98-1.78 (m, 6H), 1.51-1.26 (m, 5H). 19F NMR (282 MHz, CDCl3) δ−101.02 (t, J=6.0 Hz). LCMS (100% purity), (ESI) m/z 567.2 [M+H]+.

### Example 63

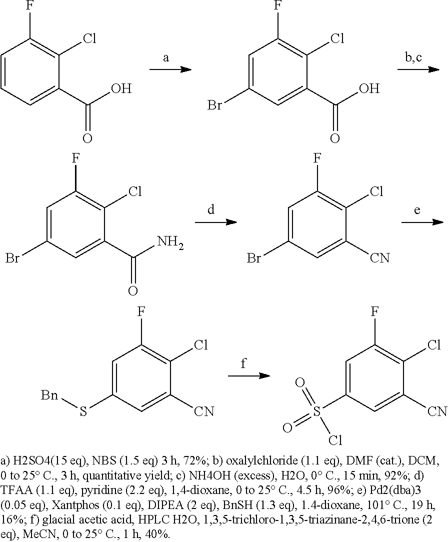

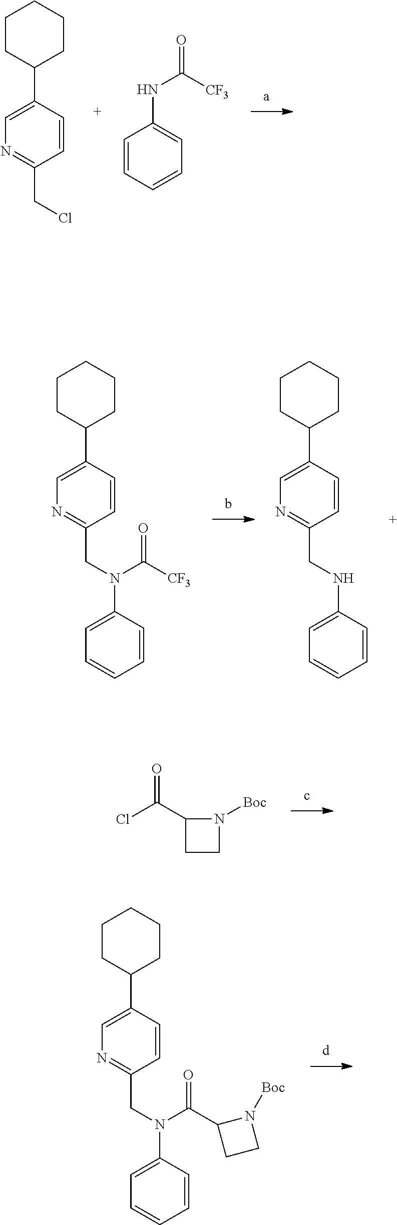 [SMILES: ClCc1ccc(C2CCCCC2)cn1 ; O=C(Nc1ccccc1)C(F)(F)F ; O=C(N(Cc1ccc(C2CCCCC2)cn1)c1ccccc1)C(F)(F)F ; c1ccc(NCc2ccc(C3CCCCC3)cn2)cc1 ; CC(C)(C)OC(=O)N1CCC1C(=O)Cl ; CC(C)(C)OC(=O)N1CCC1C(=O)N(Cc1ccc(C2CCCCC2)cn1)c1ccccc1]

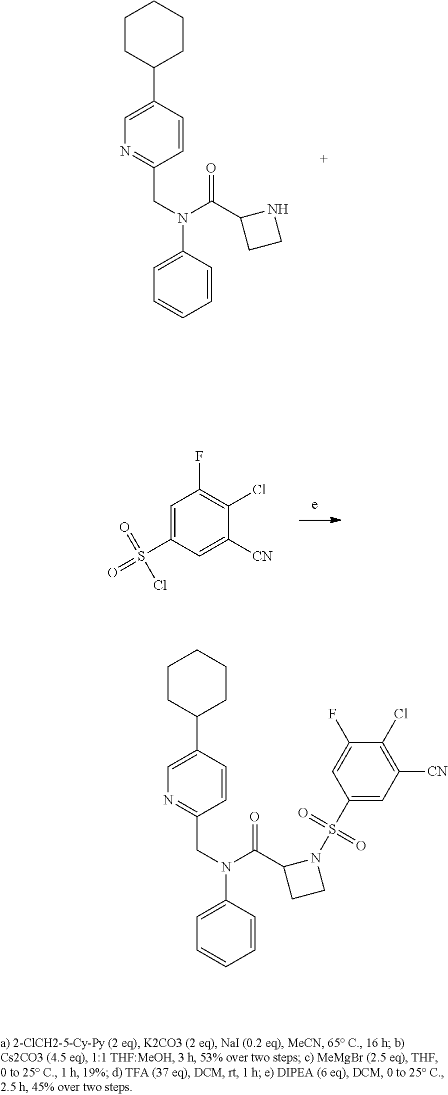 [SMILES: O=C(C1CCN1)N(Cc1ccc(C2CCCCC2)cn1)c1ccccc1 ; N#Cc1cc(S(=O)(=O)Cl)cc(F)c1Cl ; N#Cc1cc(S(=O)(=O)N2CCC2C(=O)N(Cc2ccc(C3CCCCC3)cn2)c2ccccc2)cc(F)c1Cl]

### (R)-1-((4-chloro-3-cyano-5-fluorophenyl)sulfonyl)-N-((5-cyclohexylpyridin-2-yl)methyl)-N-phenylazetidine-2-carboxamide

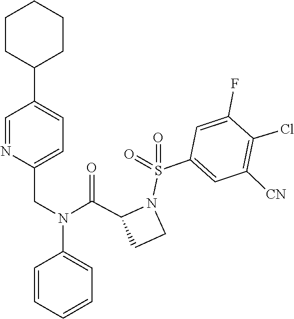 [SMILES: N#Cc1cc(S(=O)(=O)N2CC[C@@H]2C(=O)N(Cc2ccc(C3CCCCC3)cn2)c2ccccc2)cc(F)c1Cl]

Step 1. To 2-chloro-3-fluorobenzoic acid (3.08 g) was added concentrated H2SO4 (14 ml) and N-bromosuccinimide (4.709 g). The reaction mixture was heated with stirring at 60° C. for three hours under argon. Reaction was then allowed to cool to room temperature and poured onto ice water. This mixture was allowed to stir at room temperature for five minutes, and then filtered. The solid was then washed with room temperature water. The solid was then dissolved in ethyl acetate and extracted with 3M sodium hydroxide. The ethyl acetate layer was then discarded, and the aqueous layer was then acidified with 3M HCl. The aqueous layer was extracted with ethyl acetate. The combined extracts were washed with water, brine, dried over Na2SO4 and concentrated to obtain crude 5-bromo-2-chloro-3-fluorobenzoic acid contaminated with some of the other regioisomers (3.204 g, 72% yield). These isomers were separated at a later stage.

Step 2. To a solution of 5-bromo-2-chloro-3-fluorobenzoic acid (3.2 g) in dichloromethane (40 ml) was added oxalyl chloride (1.6 ml) followed by dry DMF (5 drops) under argon. The mixture was allowed to stir at room temperature for 2.5 hours and then concentrated. Dry acetonitrile (20 ml) was added, and the solution was poured onto cold concentrated ammonium hydroxide (150 ml). The mixture was allowed to reach room temperature and then stirred for 15 minutes. Water was added to the mixture and then it was extracted with ethyl acetate. The extracts were then washed with water, brine, dried over Na2SO4 and concentrated to obtain crude 5-bromo-2-chloro-3-fluorobenzamide (2.912 g, 92% yield).

Step 3. To a solution of 5-bromo-2-chloro-3-fluorobenzamide (2.912 g) in dioxane (50 ml) was added anhydrous pyridine (1.9 ml). The solution was cooled in an ice-water bath, and trifluoroacetic anhydride (1.8 ml) was added. The reaction was allowed to reach room temperature and then stirred for sixteen hours. The mixture was poured onto water and extracted with ethyl acetate. The organic extracts were then washed with sodium bicarbonate. The combined organic extracts were then washed with water, brine, dried, and concentrated to obtain crude 5-bromo-2-chloro-3-fluorobenzonitrile (2.595 g, 96% yield).

Step 4. To 5-bromo-2-chloro-3-fluorobenzonitrile (798 mg) was added Pd2(dba)3 (77 mg) and Xantphos (98 mg). The reaction vessel was then flushed with argon. To the solids was added dioxane (10 ml), followed by i-Pr2NEt (1.1 ml), and benzyl mercaptan (0.41 ml). The reaction mixture was allowed to stir at 101° C. for 19 hours. After cooling to room temperature, water was added, and the mixture was then extracted with ethyl acetate. The combined organic extracts were washed with water, brine, dried and concentrated. Purification by column chromatography (1:3 toluene:hexanes) to resolve isomeric impurities (see Step 1) gave pure 2-chloro-3-fluoro-5-(phenylthio)benzonitrile (155 mg, 16% yield).

Step 5. To a solution of 2-chloro-3-fluoro-5-(phenylthio)benzonitrile (160 mg) in HPLC acetonitrile (6 ml) was added acetic acid (0.150 ml) and HPLC water (0.72 ml). The mixture was cooled to 0° C. and 1,3,5-trichloro-1,3,5-triazinane-2,4,6-trione (260 mg). The ice bath was removed and the reaction was stirred for one hour. Added water to the reaction and extracted with ethyl acetate. The organic extracts were washed with pH 7 buffer, water, brine, dried, and concentrated. Purification by column chromatography (96:4 hexanes:ethyl acetate) gave pure 4-chloro-3-cyano-5-fluorobenzenesulfonyl chloride (60 mg, 40% yield). 1H NMR (300 MHz, CDCl3) δ 8.19-8.13 (m, 1H), 7.76-7.71 (m, 1H). 19F NMR (282 MHz, CDCl3) δ-116.62-−118.97 (m).

Step 6. To 2,2,2-trifluoro-N-phenylacetamide (460 mg) under argon was added NaI (mg) and Cs2CO3 (3.5 g). The solids were then dissolved in acetonitrile (30 ml). A 1M solution of (chloromethyl)-5-cyclohexylpyridine (3.4 ml) in toluene was added to the mixture which was then heated to 60° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. Purification by column chromatography (2:8 ethyl acetate/hexanes) gave N-((5-cyclohexylpyridin-2-yl)methyl)-2,2,2-trifluoro-N-phenylacetamide as an impure mixture with some starting material (2,2,2-trifluoro-N-phenylacetamide) (420 mg).

Step 7. To crude N-((5-cyclohexylpyridin-2-yl)methyl)-2,2,2-trifluoro-N-phenylacetamide (420 mg) under argon was added K2CO3 (326 mg) followed by THF (8 ml) and methanol (8 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave N-((5-cyclohexylpyridin-2-yl)methyl)aniline (333 mg, 53% yield over two steps). 1H NMR (300 MHz, CDCl3) δ 8.45 (d, J=2.3 Hz, 1H), 7.50 (dd, J=8.0, 2.3 Hz, 1H), 7.28 (t, J=1.6 Hz, 1H), 7.25-7.16 (m, 2H), 6.78-6.66 (m, 2H), 4.75 (s, 1H), 4.44 (d, J=4.7 Hz, 2H), 2.65-2.45 (m, 1H), 1.96-1.66 (m, 5H), 1.55-1.18 (m, 5H).

Step 8. To a stirred solution of N-((5-cyclohexylpyridin-2-yl)methyl)aniline (330 mg) in THF (8 ml) at 0° C. under argon was added a solution of 1.4M MeMgBr (1.32 ml) in 1:3 toluene:tetrahydrofuran. The reaction mixture was allowed to stir for fifteen minutes before tert-butyl (R)-2-(chlorocarbonyl)azetidine-1-carboxylate (544 mg) in THF (8 ml) was added. The ice bath was then removed and the reaction was allowed to reach room temperature. After two and a half hours saturated ammonium chloride solution was added. The reaction mixture was then extracted with ethyl acetate. The combined organic extracts were then washed with water, brine, and dried over Na2SO4. Purification by column chromatography (4:6 ethyl acetate/hexanes) gave tert-butyl (R)-2-(((5-cyclohexylpyridin-2-yl)methyl) (phenyl)carbamoyl)azetidine-1-carboxylate (102 mg, 19% yield). 1H NMR (300 MHz, CDCl3) δ 8.33 (d, J=2.1 Hz, 1H), 7.56-7.42 (m, 2H), 7.41-7.28 (m, 3H), 7.15 (d, J=14.8 Hz, 2H), 5.09 (d, J=15.0 Hz, 2H), 4.58 (s, 1H), 4.08 (td, J=8.8, 7.0 Hz, 1H), 3.75 (td, J=8.4, 5.7 Hz, 1H), 2.50 (s, 1H), 2.29-2.07 (m, 2H), 1.97-1.70 (m, 5H), 1.52-1.20 (m, 14H).

Step 9. To a solution of tert-butyl (R)-2-(((5-cyclohexylpyridin-2-yl)methyl)(phenyl)carbamoyl)azetidine-1-carboxylate in DCM (5 ml) under argon was added TFA (0.5 ml). The reaction mixture was stirred at room temperature for one hour. The reaction was then concentrated and the resulting solid ((R)—N-((5-cyclohexylpyridin-2-yl)methyl)-N-phenylazetidine-2-carboxamide TFA salt) was used directly in the next reaction.

Step 10. The solid ((R)—N-((5-cyclohexylpyridin-2-yl)methyl)-N-phenylazetidine-2-carboxamide TFA salt) was dissolved in DCM (5 ml), and DIPEA (0.105 ml) was added to the solution at 0° C. The reaction mixture was allowed to stir for fifteen minutes. 4-chloro-3-cyano-5-fluorobenzenesulfonyl chloride (30 mg) in DCM (5 ml) was added. The ice bath was removed and the reaction was allowed to warm to room temperature. After two and a half hours saturated ammonium chloride solution was added. The reaction mixture was extracted with dichloromethane. The combined organic extracts were washed with water, brine, and dried over Na2SO4. Purification by column chromatography (1:1 ethyl acetate/hexanes) gave (R)-1-((4-chloro-3-cyano-5-fluorophenyl)sulfonyl)-N-((5-cyclohexylpyridin-2-yl)methyl)-N-phenylazetidine-2-carboxamide (25 mg, 45% yield over two steps)1H NMR (500 MHz, CDCl3) δ 8.37 (d, J=2.3 Hz, 1H), 8.06 (d, J=5.0 Hz, 2H), 7.60 (d, J=8.3 Hz, 1H), 7.46-7.30 (m, 4H), 7.23-7.14 (m, 2H), 5.16-4.88 (m, 3H), 3.95 (q, J=8.0 Hz, 1H), 3.69 (td, J=8.0, 7.4, 4.0 Hz, 1H), 2.54 (s, 1H), 2.39 (p, J=8.5 Hz, 1H), 1.82 (dd, J=47.9, 10.9 Hz, 6H), 1.46-1.21 (m, 5H). 19F NMR (471 MHz, CDCl3) δ−106.79 (d, J=7.6 Hz). LCMS (100% purity), (ESI) m/z 567.2 [M+1-1]+.

### Example 64

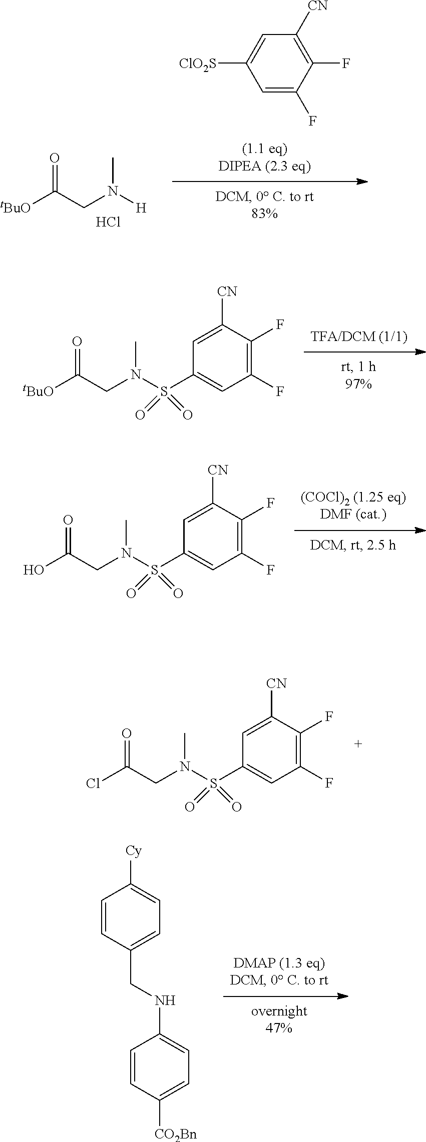

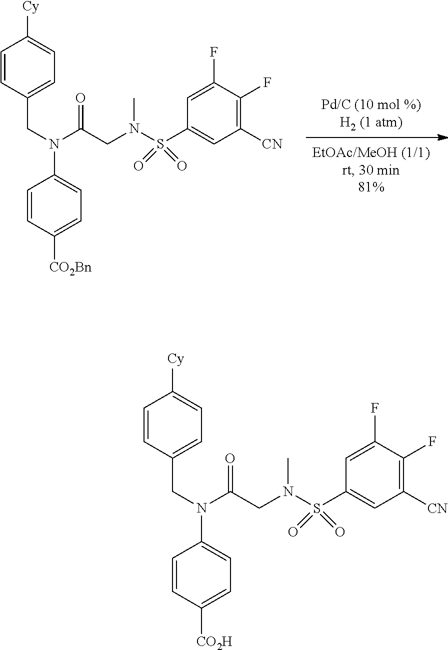

### 4-(2-((3-cyano-4,5-difluoro-N-methylphenyl)sulfonamido)-N-(4-cyclohexylbenzyl) acetamido)benzoic acid

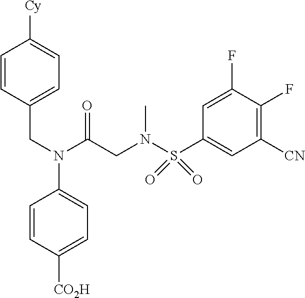

Step 1: To a solution of tert-butyl methylglycinate hydrochloride (500 mg, 2.75 mmol, 1.0 equiv) in 25 mL DCM was added DIPEA (1.1 mL, 6.33 mmol, 2.3 equiv) at 0° C. under Argon. After 5 minutes, 3-cyano-4,5-difluorobenzenesulfonyl chloride (719 mg, 3.0 mmol, 1.1 equiv) was added to the reaction at 0° C. under Argon. The reaction was allowed to warm up to room temperature and stirred at room temperature overnight. Then the reaction was quenched with saturated NH4Cl aqueous solution, extracted with DCM (3×). The combined organic extracts were washed with saturated brine, dried over anhydrous sodium sulfate, and concentrated under reduced pressure. The resulting residue was purified by flash chromatography (eluent: hexane/EtOAc 10/1) to provide tert-butyl N-((3-cyano-4,5-difluorophenyl)sulfonyl)-N-methylglycinate as white solid (950 mg, 83%). 1H NMR (300 MHz, CDCl3) δ 7.98-7.83 (m, 2H), 4.01 (s, 2H), 2.96 (s, 3H), 1.41 (s, 9H). 19F NMR (282 MHz, CDCl3) δ−123.05-−123.17 (m), −130.04-−130.14 (m).

Step 2: To a solution of tert-butyl N-((3-cyano-4,5-difluorophenyl)sulfonyl)-N-methylglycinate (850 mg, 2.45 mmol, 1.0 equiv) in 25 mL DCM was added TFA (8.5 mL) at room temperature under Argon. The reaction was stirred at room temperature for 3 h. Then the reaction was concentrated under reduced pressure, diluted with dry DCE and concentrated again. The residue was dried under high vacuum to provide N-((3-cyano-4,5-difluorophenyl)sulfonyl)-N-methylglycine as white solid (688 mg, 97%). 1H NMR (300 MHz, CD3OD) δ 8.21-8.08 (m, 2H), 4.10 (s, 2H), 2.94 (s, 3H). 19F NMR (282 MHz, CD3OD) δ−127.72-−127.84 (m), −133.90-−134.00 (m).

Step 3: To a solution of N-((3-cyano-4,5-difluorophenyl)sulfonyl)-N-methylglycine (584 mg, 2.01 mmol, 1.0 equiv) in 40 mL DCM was added oxalyl chloride (0.26 mL, 3.02 mmol, 1.5 equiv) and 1 drop of DMF at room temperature under Argon. After 2 h, the reaction was concentrated under reduced pressure, diluted with dry DCE and concentrated again. The residue was dried under high vacuum and used directly without further purification.

Step 4: To a solution of benzyl 4-((4-cyclohexylbenzyl)amino)benzoate (575 mg, 1.44 mmol, 1.0 equiv) and N-((3-cyano-4,5-difluorophenyl)sulfonyl)-N-methylglycinoyl chloride (1.88 mmol, 1.3 equiv) in 15 mL DCM was added DMAP (230 mg, 1.88 mmol, 1.3 equiv) at 0° C. under Argon. The reaction was allowed to warm up to room temperature and stirred at room temperature overnight. Then the reaction was quenched with water, extracted with DCM (3×). The combined organic extracts were washed with saturated brine, dried over anhydrous sodium sulfate, and concentrated under reduced pressure. The resulting residue was purified by flash chromatography (eluent: hexane/EtOAc 2/1) to provide benzyl 4-(2-((3-cyano-4,5-difluoro-N-methylphenyl)sulfonamido)-N-(4-cyclohexylbenzyl)acetamido) benzoate as white foam (455 mg, 47%). 1H NMR (300 MHz, CDCl3) δ 8.15-8.07 (m, 2H), 8.01-7.91 (m, 2H), 7.52-7.34 (m, 5H), 7.17-7.05 (m, 4H), 7.00 (d, J=7.8 Hz, 2H), 5.40 (s, 2H), 4.76 (s, 2H), 3.88 (s, 2H), 2.95 (s, 3H), 2.55-2.43 (m, 1H), 1.93-1.71 (m, 6H), 1.46-1.35 (m, 4H). 19F NMR (282 MHz, CDCl3) δ−123.15 (dt, J=19.8, 5.7 Hz), −130.27 (dd, J=20.0, 8.0 Hz).

Step 5: benzyl 4-(2-((3-cyano-4,5-difluoro-N-methylphenyl)sulfonamido)-N-(4-cyclohexylbenzyl)acetamido) benzoate (20 mg) and Pd/C (2 mg) were dissolved in EtOAc/MeOH (1 mL, 1/1) under hydrogen gas (1 atm). After 30 minutes, the catalyst was filtered off through a celite pad and washed with ethyl acetate. The combined solvent was concentrated under reduced pressure and the residue was purified by preparative TLC plates (eluent: DCM/MeOH=15/1) to obtain 4-(2-((3-cyano-4,5-difluoro-N-methylphenyl)sulfonamido)-N-(4-cyclohexylbenzyl)acetamido)benzoic acid as white solid (14 mg, 81%). 1H NMR (300 MHz, CD3OD) δ 8.16-7.94 (m, 4H), 7.24-7.09 (m, 4H), 7.02 (d, J=7.8 Hz, 2H), 4.79 (s, 2H), 3.93 (s, 2H), 2.93 (s, 3H), 2.47 (s, 1H), 1.97-1.55 (m, 6H), 1.52-1.36 (m, 4H). 19F NMR (282 MHz, CD3OD) δ−127.73 (dt, J=19.8, 5.7 Hz), −133.89 (dd, J=19.3, 9.3 Hz). LRMS (ESI) m/z 582.2 [M+H]+, 604.2 [M+Na]+; HRMS (ESI) m/z 582.1847 [M+H]+, 604.1679 [M+Na]+; Purity 100%.

### Example 65

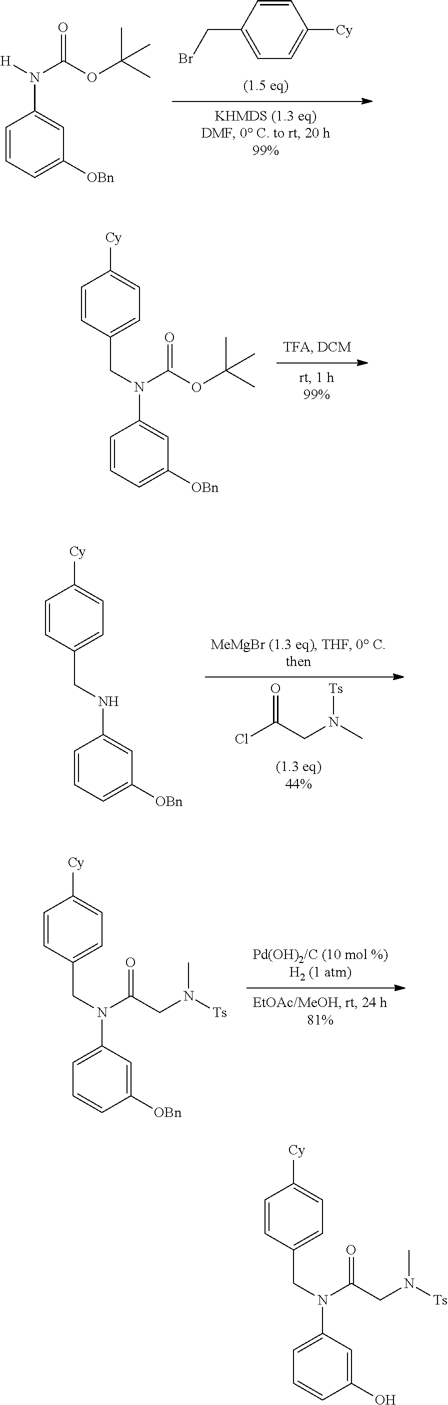

### N-(4-cyclohexylbenzyl)-24(N,4-dimethylphenyl)sulfonamido)-N-(3-hydroxyphenyl) acetamide

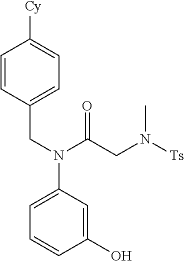

Step 1: To a solution of tert-butyl (3-(benzyloxy)phenyl)carbamate (350 mg, 1.17 mmol, 1.0 equiv) in 6 mL DMF was added KHMDS (1.4 mL, 1.40 mmol, 1.2 equiv, 1.0 M in THF) at 0° C. dropwise under Argon. 10 minutes later, 1-(bromomethyl)-4-cyclohexylbenzene (415 mg, 1.64 mmol, 1.4 equiv) was added to the reaction mixture. The reaction was allowed to warm up to room temperature and stirred at room temperature for 20 h. Then the reaction was quenched with saturated NH4Cl aqueous solution, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 50/1) to provide tert-butyl (3-(benzyloxy)phenyl)(4-cyclohexylbenzyl) carbamate as colorless oil (580 mg, 99%). 1H NMR (300 MHz, CDCl3) δ 7.42-7.28 (m, 5H), 7.21-7.09 (m, 5H), 6.82-6.73 (m, 3H), 4.96 (s, 2H), 4.76 (s, 2H), 2.53-2.41 (m, 1H), 1.94-1.65 (m, 6H), 1.41 (s, 9H), 1.45-1.32 (m, 4H).

Step 2: To a solution of tert-butyl (3-(benzyloxy)phenyl)(4-cyclohexylbenzyl) carbamate (550 mg, 1.17 mmol) in 10 mL DCM was added TFA (3.5 mL). The reaction was stirred at room temperature for 1 h. Then the reaction was concentrated under reduced pressure, then quenched with saturated NaHCO3 aqueous solution to pH 8. The reaction was extracted with DCM (3×). The combined organic extracts were washed with saturated brine, dried over anhydrous sodium sulfate, and concentrated under reduced pressure to obtain 3-(benzyloxy)-N-(4-cyclohexylbenzyl)aniline as light yellow oil (438 mg, 99%). 1H NMR (300 MHz, CDCl3) δ 7.64-7.29 (m, 7H), 7.29-7.20 (m, 2H), 7.20-7.08 (m, 1H), 6.47-6.27 (m, 3H), 5.07 (d, J=3.7 Hz, 2H), 4.31 (d, J=3.1 Hz, 2H), 3.94 (s, 1H), 2.64-2.48 (m, 1H), 2.02-1.76 (m, 5H), 1.59-1.31 (m, 5H).

Step 3: To a solution of 3-(benzyloxy)-N-(4-cyclohexylbenzyl)aniline (230 mg, 0.62 mmol, 1.0 equiv) in 6 mL THF was added MeMgBr (0.6 mL, 0.80 mmol, 1.3 equiv, 1.4 M in THF/toluene) at 0° C. under Argon. 10 minutes later, a solution of N-methyl-N-tosylglycinoyl chloride (243 mg, 0.93 mmol, 1.5 equiv) in 2 mL THF was added to the reaction. The reaction was allowed to warm up to room temperature and stirred for 30 minutes. Then the reaction was quenched with saturated NH4Cl aq, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 5/1) to provide N-(3-(benzyloxy)phenyl)-N-(4-cyclohexylbenzyl)-2-((N,4-dimethylphenyl)sulfonamido)acetamide as light yellow oil (162 mg, 44%). 1H NMR (300 MHz, CDCl3) δ 7.67 (d, J=8.2 Hz, 2H), 7.48-7.33 (m, 5H), 7.32-7.24 (m, 3H), 7.15-6.95 (m, 5H), 6.63 (d, J=7.9 Hz, 1H), 6.54 (s, 1H), 4.96 (s, 2H), 4.76 (s, 2H), 3.76 (s, 2H), 2.87 (s, 3H), 2.55-2.47 (m, 1H), 2.43 (s, 3H), 1.93-1.71 (m, 6H), 1.46-1.34 (m, 4H).

Step 4: N-(3-(benzyloxy)phenyl)-N-(4-cyclohexylbenzyl)-2-((N,4-dimethyl phenyl)sulfonamido)acetamide (160 mg) and Pd(OH)2/C (16 mg) were dissolved in EtOAc/MeOH (5 mL, 1/1) under hydrogen gas (1 atm). After 24 h, the catalyst was filtered off through a celite pad and washed with ethyl acetate. The combined solvent was concentrated under reduced pressure and the residue was purified by flash chromatography (eluent: Hexane/EtOAc 2/1) to obtain N-(4-cyclohexylbenzyl)-2-((N,4-dimethylphenyl)sulfonamido)-N-(3-hydroxyphenyl)acetamide as white foam (110 mg, 81%). 1H NMR (300 MHz, CD3OD) δ 7.64 (d, J=8.2 Hz, 2H), 7.27-7.13 (m, 3H), 7.13-7.00 (m, 4H), 6.87 (d, J=8.1 Hz, 1H), 6.66 (s, 1H), 6.50 (d, J=7.6 Hz, 1H), 4.78 (s, 2H), 3.81 (s, 2H), 2.86 (s, 3H), 2.52-2.42 (m, 1H), 2.40 (s, 3H), 1.92-1.64 (m, 5H), 1.47-1.18 (m, 5H). LRMS (ESI) m/z 507.2 [M+H]+, 529.2 [M+Na]+; HRMS (ESI) m/z 507.2328 [M+H]+, 529.2149 [M+Na]+; Purity 100%.

### Example 66

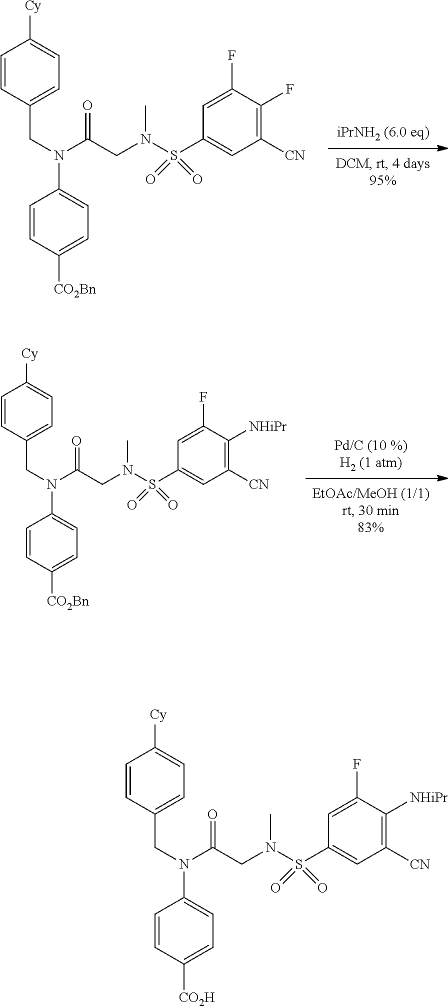

### 4-(2-((3-cyano-5-fluoro-4-(isopropylamino)-N-methylphenyl)sulfonamido)-N-(4-cyclohexylbenzyl)acetamido)benzoic acid

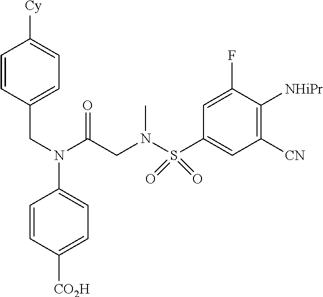

Step 1: To a solution of benzyl 4-(2-((3-cyano-4,5-difluoro-N-methylphenyl) sulfonamido)-N-(4-cyclohexylbenzyl)acetamido)benzoate (55 mg, 0.08 mmol, 1.0 equiv) in 3 mL DCM was added iPrNH2 (42 μL, 0.49 mmol, 6.0 equiv) at room temperature under Argon. The reaction was stirred at room temperature for 4 days. Then the reaction concentrated under reduced pressure. The resulting residue was purified by preparative TLC plates (eluent: hexane/EtOAc 2/1) to provide benzyl 4-(2-((3-cyano-5-fluoro-4-(isopropylamino)-N-methylphenyl)sulfonamido)-N-(4-cyclohexylbenzyl)acetamido)benzoate as colorless oil (55 mg, 95%). 1H NMR (300 MHz, CDCl3) δ 8.11-8.04 (m, 2H), 7.71-7.65 (m, 1H), 7.53 (dd, J=12.0, 2.1 Hz, 1H), 7.49-7.30 (m, 5H), 7.10 (t, J=8.3 Hz, 4H), 7.02 (d, J=8.1 Hz, 2H), 5.37 (s, 2H), 4.80 (s, 2H), 4.73-4.60 (m, 1H), 4.51-4.38 (m, 1H), 3.80 (s, 2H), 2.89 (s, 3H), 2.51-2.41 (m, 1H), 1.91-1.67 (m, 6H), 1.47-1.34 (m, 4H), 1.32 (d, J=6.3 Hz, 6H).

Step 2: Benzyl 4-(2-((3-cyano-5-fluoro-4-(isopropylamino)-N-methylphenyl) sulfonamido)-N-(4-cyclohexylbenzyl)acetamido)benzoate (83 mg) and Pd/C (8.3 mg) were dissolved in EtOAc/MeOH (5 mL, 1/1) under hydrogen gas (1 atm). After 30 minutes, the catalyst was filtered off through a celite pad and washed with ethyl acetate. The combined solvent was concentrated under reduced pressure and the residue was purified by preparative TLC plates (eluent: DCM/MeOH 10/1) to obtain 4-(2-((3-cyano-5-fluoro-4-(isopropylamino)-N-methylphenyl)sulfonamido)-N-(4-cyclohexylbenzyl)acetamido)benzoic acid as white solid (56 mg, 83%). 1H NMR (300 MHz, CD3OD) δ 7.64 (d, J=8.2 Hz, 2H), 7.27-7.13 (m, 3H), 7.13-7.00 (m, 4H), 6.87 (d, J=8.1 Hz, 1H), 6.66 (s, 1H), 6.50 (d, J=7.6 Hz, 1H), 4.78 (s, 2H), 3.81 (s, 2H), 2.86 (s, 3H), 2.52-2.42 (m, 1H), 2.40 (s, 3H), 1.92-1.64 (m, 5H), 1.47-1.18 (m, 5H). LRMS (ESI) m/z 621.2 [M+H]+, 643.2 [M+Na]+; HRMS (ESI) m/z 621.2547 [M+H]+, 643.2369 [M+Na]+; Purity 100%.

### Example 67

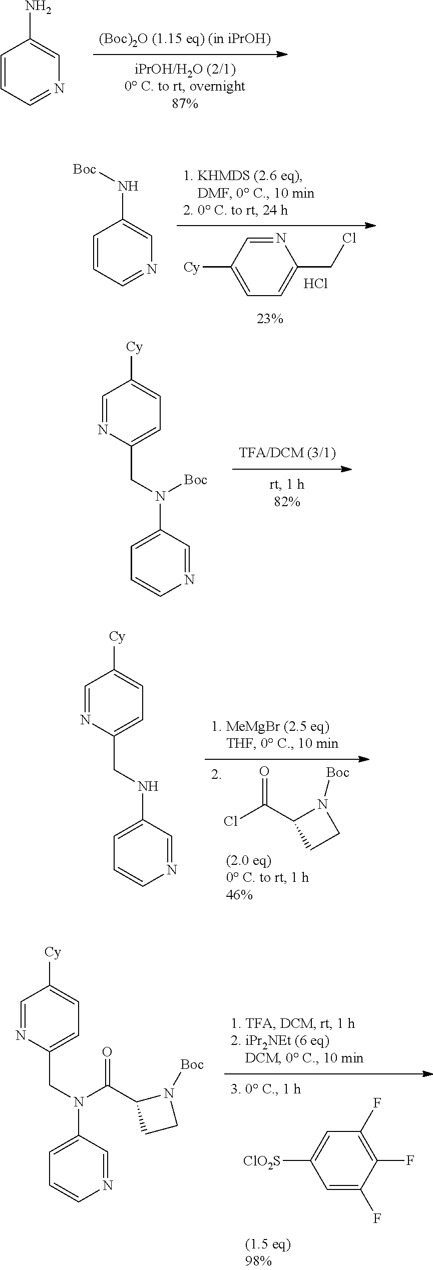

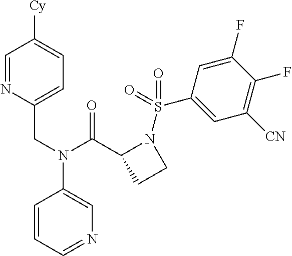

### (R)-1-((3-cyano-4,5-difluorophenyl)sulfonyl)-N-((5-cyclohexylpyridin-2-yl)methyl)-N-(pyridin-3-yl)azetidine-2-carboxamide

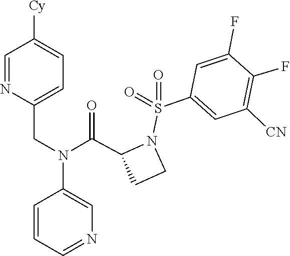

Step 1: To a solution of pyridin-3-amine (450 mg, 4.78 mmol, 1.0 equiv) in 3 mL iPrOH/H2O (2/1) was added the solution of (Boc)20 (1.26 mL, 5.50 mmol, 1.15 eq) in 1 mL iPrOH dropwise at 0° C. under Argon. Upon completion of the addition, the reaction was allowed to warm up to room temperature and stirred for overnight. Then iPrOH was removed under reduced vacuum. The residue was diluted with EtOAc, washed with water, saturated NaHCO3 aq and brine, dried over anhydrous sodium sulfate, and concentrated under reduced pressure. The resulting residue was purified by flash chromatography (eluent: hexane/EtOAc 1.5/1) to provide tert-butyl pyridin-3-ylcarbamate as white solid (808 mg, 87%). 1H NMR (300 MHz, CDCl3) δ 8.50 (d, J=2.3 Hz, 1H), 8.30 (dd, J=4.6, 1.1 Hz, 1H), 8.05 (d, J=6.2 Hz, 1H), 7.28 (dd, J=7.8, 5.2 Hz, 1H) 6.80 (s, 1H), 1.55 (s, 9H).

Step 2: To a solution of tert-butyl pyridin-3-ylcarbamate (200 mg, 1.03 mmol, 1.0 equiv) in 6 mL DMF was added KHMDS (2.7 mL, 2.67 mmol, 2.6 equiv, 1.0 M in THF) at 0° C. dropwise under Argon. 10 minutes late, 2-(chloromethyl)-5-cyclohexylpyridine hydrochloride (304.3 mg, 1.24 mmol, 1.2 equiv) was added to the reaction mixture. The reaction was allowed to warm up to room temperature and stirred at room temperature for 24 h. Then the reaction was quenched with saturated NH4Cl aqueous solution, extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone 10/1) to provide tert-butyl ((5-cyclohexylpyridin-2-yl)methyl)(pyridin-3-yl)carbamate as light yellow oil (86 mg, 23%). 1H NMR (300 MHz, CDCl3) δ 8.53 (s, 1H), 8.40-8.28 (m, 2H), 7.63 (d, J=7.8 Hz, 1H), 7.47 (dt, J=8.0, 1.9 Hz, 1H), 7.25-7.14 (m, 2H), 4.90 (d, J=2.1 Hz, 2H), 2.55-2.41 (m, 1H), 1.92-1.65 (m, 6H), 1.45-1.29 (m, 13H).

Step 3: To a solution of tert-butyl ((5-cyclohexylpyridin-2-yl)methyl)(pyridin-3-yl)carbamate (86 mg, 0.23 mmol) in 3 mL DCM was added TFA (1 mL). The reaction was stirred at room temperature for 1 h. Then the reaction was concentrated under reduced pressure, then quenched with saturated NaHCO3 aqueous solution to pH 8. The reaction was extracted with DCM (3×). The combined organic extracts were washed with saturated brine, dried over anhydrous sodium sulfate, and concentrated under reduced pressure. The resulting residue was purified by preparative TLC plates (eluent: hexane/acetone 2/1) to provide N-((5-cyclohexylpyridin-2-yl)methyl)pyridin-3-amine as white solid (52 mg, 83%). 1H NMR (500 MHz, CDCl3) δ 8.43 (d, J=1.8 Hz, 1H), 8.13 (d, J=2.7 Hz, 1H), 7.97 (dd, J=4.7, 1.1 Hz, 1H), 7.51 (dd, J=8.0, 2.2 Hz, 1H), 7.24 (d, J=8.0 Hz, 1H), 7.14 (dd, J=8.3, 4.7 Hz, 1H), 7.00 (dd, J=8.3, 1.6 Hz, 1H), 5.12 (s, 1H), 4.42 (d, J=3.1 Hz, 2H), 2.58-2.50 (m, 1H), 1.90-1.74 (m, 5H), 1.46-1.21 (m, 5H).

Step 4: To a solution of (R)-1-(tert-butoxycarbonyl)azetidine-2-carboxylic acid (77 mg, 0.38 mmol, 2.0 equiv) in 3 mL DCM was added DMF (1 drop, cat.) and oxalyl chloride (40 μL, 0.48 mmol, 2.5 equiv) dropwise under Argon. The reaction was stirred at room temperature for 1.5 h. Then the mixture was concentrated under reduced pressure, diluted with dry DCE and concentrated again. The residue was dried under high vacuum for 30 minutes and used directly for the next step.

To a solution of N-((5-cyclohexylpyridin-2-yl)methyl)pyridin-3-amine (51 mg, 0.19 mmol, 1.0 equiv) in 2 mL THF was added MeMgBr (0.34 mL, 0.48 mmol, 2.5 equiv, 1.4 M in THF/toluene) at 0° C. under Argon. 10 minutes later, a solution of the above residue in 2 mL THF was added to the reaction. The reaction was allowed to warm up to room temperature and stirred for 1 h. Then the reaction was quenched with saturated NH4Cl aqueous solution, extracted with ethyl acetate (3×). The combined organic extracts were washed with saturated brine, dried over anhydrous sodium sulfate, and concentrated under reduced pressure. The resulting residue was purified by preparative TLC plates (eluent: hexane/acetone 2/1) to provide tert-butyl (R)-2-(((5-cyclohexylpyridin-2-yl)methyl) (pyridin-3-yl)carbamoyl)azetidine-1-carboxylate as colorless oil (40 mg, 46%). 1H NMR (500 MHz, CDCl3) δ 8.53 (s, 1H), 8.41 (s, 1H), 8.29 (s, 1H), 7.62 (s, 1H), 7.47 (dd, J=8.0, 2.0 Hz, 1H), 7.39 (d, J=7.6 Hz, 1H), 7.30 (s, 1H), 5.12-4.80 (m, 2H), 4.48 (s, 1H), 4.09-4.00 (m, 1H), 3.86-3.59 (m, 1H), 2.53-2.43 (m, 1H), 2.21-2.12 (m, 2H), 1.86-1.70 (m, 5H), 1.57-1.28 (m, 14H).

Step 5: To a solution of tert-butyl (R)-2-(((5-cyclohexylpyridin-2-yl)methyl)(pyridin-3-yl)carbamoyl)azetidine-1-carboxylate (35 mg, 0.08 mmol) in 1 mL DCM was added TFA (0.3 mL). The reaction was stirred at room temperature for 1 h. Then the reaction was concentrated under reduced pressure, diluted with dry DCE and concentrated again. The residue was dried under high vacuum for 30 minutes and used directly for the next step.

To a solution of the above residue in 1 mL DCM was added DIPEA (77 μL, 0.47 mmol, 6.0 equiv) at 0° C. under Argon. After 10 minutes, a solution of 3-cyano-4,5-difluorobenzenesulfonyl chloride (28 mg, 0.12 mmol, 1.5 equiv) in 1 mL DCM was added dropwise under Argon at 0° C. The reaction was stirred at 0° C. for 1 h. Then the reaction was quenched with saturated NH4Cl aqueous solution, and extracted with DCM (3×). The combined organic extracts were washed with saturated brine, dried over anhydrous sodium sulfate, and concentrated under reduced pressure. The resulting residue was purified by preparative TLC plates (eluent: DCM/MeOH 10/1) to provide (R)-1-((3-cyano-4,5-difluorophenyl)sulfonyl)-N-((5-cyclohexylpyridin-2-yl)methyl)-N-(pyrid in-3-yl)azetidine-2-carboxamide as white solid (42 mg, 98%). 1H NMR (500 MHz, CDCl3) δ 8.64 (d, J=4.3 Hz, 1H), 8.46 (s, 1H), 8.37 (s, 1H), 8.21-8.14 (m, 1H), 8.11-8.05 (m, 1H), 7.67 (d, J=7.9 Hz, 1H), 7.53 (d, J=7.9 Hz, 1H), 7.38 (dd, J=7.9, 4.7 Hz, 1H), 7.28 (s, 1H), 5.08-4.88 (m, 3H), 4.07-3.96 (m, 1H), 3.70-3.64 (m, 1H), 2.59-2.47 (m, 1H), 2.42-2.34 (m, 1H), 1.96-1.74 (m, 7H), 1.44-1.36 (m, 4H). 19F NMR (471 MHz, CDCl3) δ−122.86 (dt, J=19.8, 5.2 Hz), −129.92 (dd, J=19.8, 8.9 Hz). LRMS (ESI) m/z 552.3 [M+H]+; HRMS (ESI) m/z 552.1866 [M+H]+, 574.1685 [M+Na]+; Purity 100%.

### Example 68

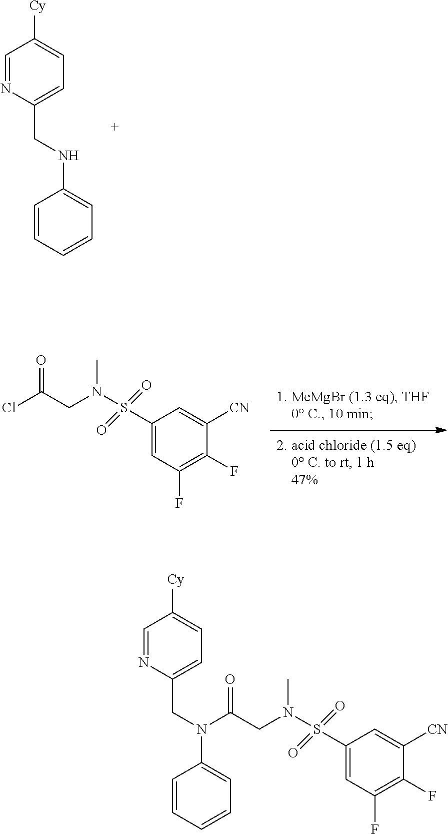

### 2-((3-cyano-4,5-difluoro-N-methylphenyl)sulfonamido)-N-((5-cyclohexylpyridin-2-yl)methyl)-N-phenylacetamide

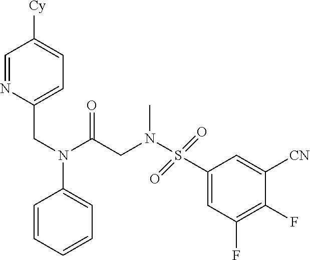

Step 1: To a solution of N-((5-cyclohexylpyridin-2-yl)methyl)aniline (50 mg, 0.19 mmol, 1.0 equiv) was added MeMgBr (0.17 mL, 0.24 mmol, 1.3 equiv, 1.4 M in THF/toluene) dropwise at 0° C. under Argon. After 10 minutes, the solution of N-((3-cyano-4,5-difluorophenyl)sulfonyl)-N-methylglycinoyl chloride (0.24 mmol, 1.3 equiv) in 1 mL THF was added at 0° C. under Argon. The reaction was allowed to warm up to room temperature and stirred at room temperature for 1 h. Then the reaction was quenched with water, extracted with EtOAc (3×). The combined organic extracts were washed with saturated brine, dried over anhydrous sodium sulfate, and concentrated under reduced pressure. The resulting residue was purified by preparative TLC plates (eluent: hexane/EtOAc 1/1) to provide 2-((3-cyano-4,5-difluoro-N-methylphenyl)sulfonamido)-N-((5-cyclohexylpyridin-2-yl)methy 1)-N-phenylacetamide as white solid (48 mg, 47%). 1H NMR (500 MHz, CDCl3) δ 8.38 (s, 1H), 8.01-7.88 (m, 2H), 7.52 (d, J=6.6 Hz, 1H), 7.46-7.33 (m, 3H), 7.19 (d, J=6.6 Hz, 2H), 7.15 (d, J=7.5 Hz, 1H), 4.90 (s, 2H), 3.96 (s, 2H), 2.94 (s, 3H), 2.62-2.45 (m, 1H), 1.89-1.74 (m, 5H), 1.44-1.22 (m, 5H). 19F NMR (471 MHz, CDCl3) δ−123.42 (dt, J=19.6, 5.0 Hz), −130.43 (dd, J=19.4, 8.7 Hz). LRMS (ESI) m/z 539.3 [M+H]+; HRMS (ESI) m/z 539.1915 [M+H]+, 561.1735 [M+Na]+; Purity 100%.

### Example 69

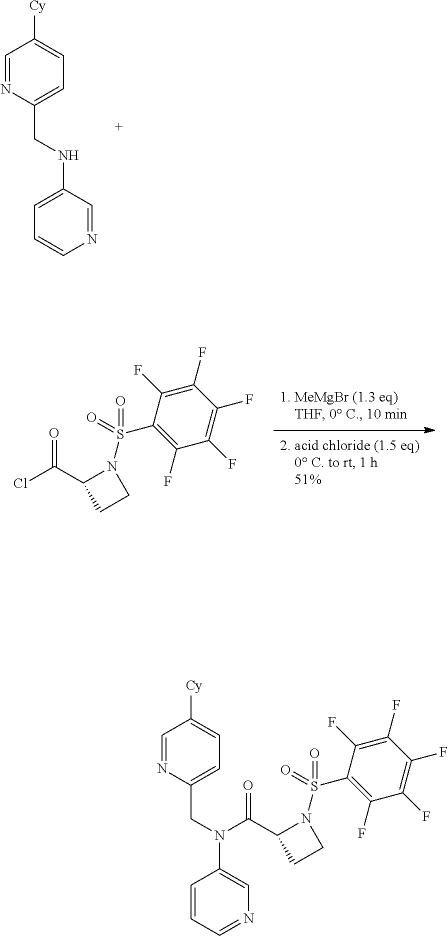

(R)—N-((5-cyclohexylpyridin-2-yl)methyl)-1-((perfluorophenyl)sulfonyl)-N-(pyridin-3-yl)azetidine-2-carboxamide

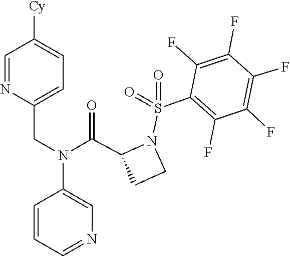

Step 1: To a solution of N-((5-cyclohexylpyridin-2-yl)methyl)pyridin-3-amine (50 mg, 0.19 mmol, 1.0 equiv) was added MeMgBr (0.17 mL, 0.24 mmol, 1.3 equiv, 1.4 M in THF/toluene) dropwise at 0° C. under Argon. After 10 minutes, (R)-1-((perfluorophenyl)sulfonyl)azetidine-2-carbonyl chloride (98 mg, 0.28 mmol, 1.5 equiv) was added at 0° C. under Argon. The reaction was allowed to warm up to room temperature and stirred at room temperature for 1 h. Then the reaction was quenched with water, extracted with EtOAc (3×). The combined organic extracts were washed with saturated brine, dried over anhydrous sodium sulfate, and concentrated under reduced pressure. The resulting residue was purified by flash chromatography (eluent: hexane/acetone 3/1) to provide (R)—N-((5-cyclohexylpyridin-2-yl)methyl)-1-((perfluorophenyl)sulfonyl)-N-(pyridin-3-yl)azetidine-2-carboxamide as light yellow gum (55 mg, 51%). 1H NMR (500 MHz, CDCl3) δ 8.59 (d, J=3.3 Hz, 1H), 8.39 (s, 1H), 8.32 (s, 1H), 7.61 (d, J=7.3 Hz, 1H), 7.51 (d, J=7.5 Hz, 1H), 7.39-7.32 (m, 1H), 7.20 (d, J=7.7 Hz, 1H), 4.96-4.86 (m, 3H), 4.17-4.09 (m, 1H), 4.07-4.01 (m, 1H), 2.56-2.45 (m, 1H), 2.38-2.28 (m, 1H), 2.03-1.94 (m, 1H), 1.92-1.71 (m, 6H), 1.43-1.33 (m, 4H). LRMS (ESI) m/z 581.3 [M+H]+; HRMS (ESI) m/z 581.1634 [M+H]+, 603.1453 [M+Na]+; Purity 100%.

### Example 70

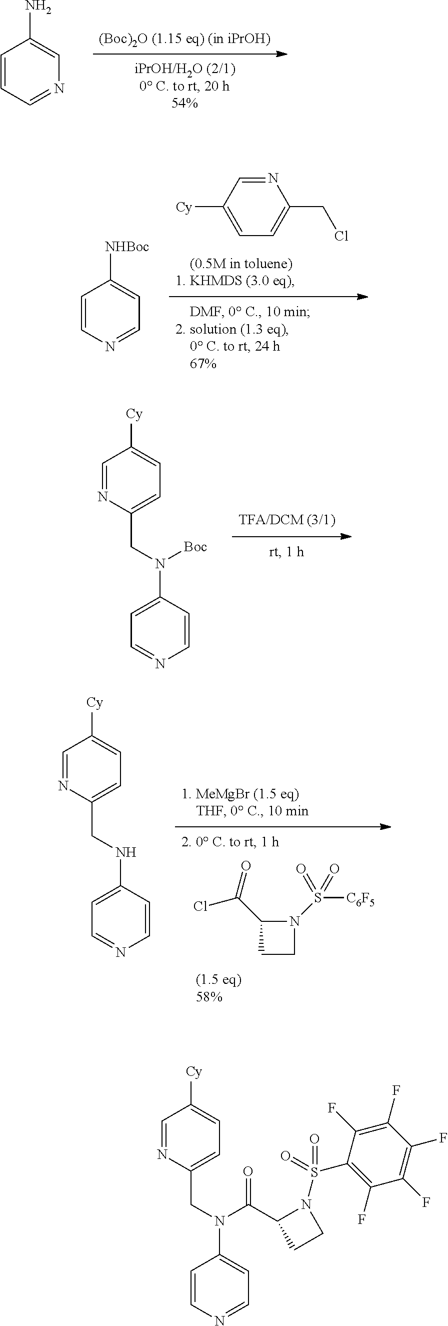

### (R)—N-((5-cyclohexylpyridin-2-yl)methyl)-1-((perfluorophenyl)sulfonyl)-N-(pyridin-4-yl)azetidine-2-carboxamide

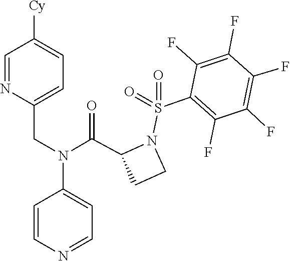

Step 1: To a solution of pyridin-4-amine (600 mg, 6.38 mmol, 1.0 equiv) in 6 mL iPrOH/H2O (2/1) at 0° C. was added a solution of (Boc)20 (1.68 mL, 7.33 mmol, 1.15 equiv) in 2 mL iPrOH dropwise. Then the reaction was allowed to warm up to room temperature and stirred at room temperature for 20 h. iPrOH was removed under reduced vacuum. The residue was diluted with EtOAc, washed with water, saturated NaHCO3 aqueous solution and brine, dried over anhydrous sodium sulfate, and concentrated under reduced pressure. The resulting residue was purified by flash chromatography (eluent: hexane/EtOAc/MeOH 1/1/5%) to provide tert-butyl pyridin-4-ylcarbamate as white solid (650 mg, 54%). 1H NMR (500 MHz, CDCl3) δ 8.43 (d, J=6.2 Hz, 2H), 7.33 (d, J=6.1 Hz, 2H), 7.04 (s, 1H), 1.52 (s, 9H).

Step 2: To a solution of tert-butyl pyridin-4-ylcarbamate (235 mg, 1.21 mmol, 1.0 equiv) in 12 mL DMF was added KHMDS (1.6 mL, 1.57 mmol, 1.3 equiv, 1.0 M in THF) at 0° C. dropwise under Argon. 10 minutes late, 2-(chloromethyl)-5-cyclohexylpyridine (3.2 mL, 1.57 mmol, 1.3 equiv, 0.5 M in toluene) was added to the reaction mixture. The reaction was allowed to warm up to room temperature and stirred at room temperature for 24 h. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone 5/1) to provide tert-butyl ((5-cyclohexylpyridin-2-yl)methyl)(pyridin-4-yl)carbamate as red oil (298 mg, 67%). 1H NMR (500 MHz, CDCl3) δ 8.44 (d, J=6.2 Hz, 2H), 8.40 (d, J=1.9 Hz, 1H), 7.48 (dd, J=8.0, 2.1 Hz, 1H), 7.33 (dd, J=5.0, 1.2 Hz, 2H), 7.13 (d, J=8.0 Hz, 1H), 4.98 (s, 2H), 2.88-2.27 (m, 1H), 2.05-1.69 (m, 5H), 1.62-1.28 (m, 14H).

Step 3: To a solution of tert-butyl ((5-cyclohexylpyridin-2-yl)methyl)(pyridin-4-yl)carbamate (30 mg, 0.08 mmol) in 1 mL DCM was added TFA (0.3 mL). The reaction was stirred at room temperature for 1 h. Then the reaction was concentrated under reduced pressure, then quenched with saturated Na2CO3 aqueous solution to pH 8. The reaction was extracted with DCM (3×). The combined organic extracts were washed with saturated brine, dried over anhydrous sodium sulfate, and concentrated under reduced pressure. The resulting residue was used directly for next step without further purification.

To a solution of the above residue (20 mg, 1.0 equiv) in 1 mL THF was added MeMgBr (80 μL, 0.11 mmol, 1.5 equiv, 1.4 M in THF/toluene) dropwise at 0° C. under Argon. After 10 minutes, (R)-1-((perfluorophenyl)sulfonyl)azetidine-2-carbonyl chloride (39 mg, 0.11 mmol, 1.5 equiv) was added at 0° C. under Argon. The reaction was allowed to warm up to room temperature and stirred at room temperature for 1 h. Then the reaction was quenched with water and extracted with EtOAc (3×). The combined organic extracts were washed with saturated brine, dried over anhydrous sodium sulfate, and concentrated under reduced pressure. The resulting residue was purified by preparative TLC plates (eluent: hexane/acetone 8/7) to provide (R)—N-((5-cyclohexylpyridin-2-yl)methyl)-1-((perfluorophenyl)sulfonyl)-N-(pyridin-4-yl)azetidine-2-carboxamide as light yellow gum (25 mg, 58%). 1H NMR (500 MHz, CDCl3) δ 8.63 (s, 2H), 8.34 (s, 1H), 7.50 (d, J=7.8 Hz, 1H), 7.25-7.10 (m, 3H), 5.21-5.06 (m, 1H), 4.91 (s, 2H), 4.26-4.13 (m, 1H), 4.12-4.03 (m, 1H), 2.60-2.46 (m, 1H), 2.39-2.30 (m, 1H), 2.16-2.05 (m, 1H), 1.91-1.72 (m, 5H), 1.55-1.31 (m, 5H). LRMS (ESI) m/z 581.2 [M+H]+; HRMS (ESI) m/z 581.1634 [M+H]+, 603.1450 [M+Na]+; Purity 100%.

### Example 71

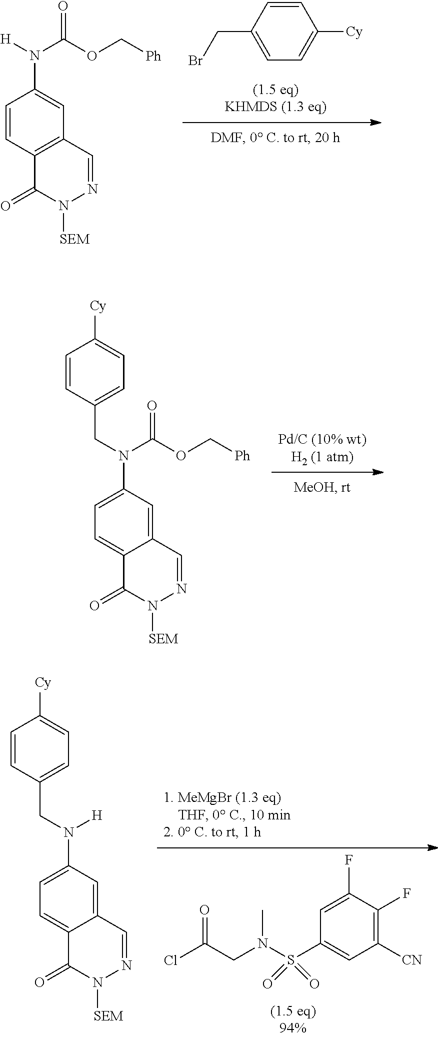

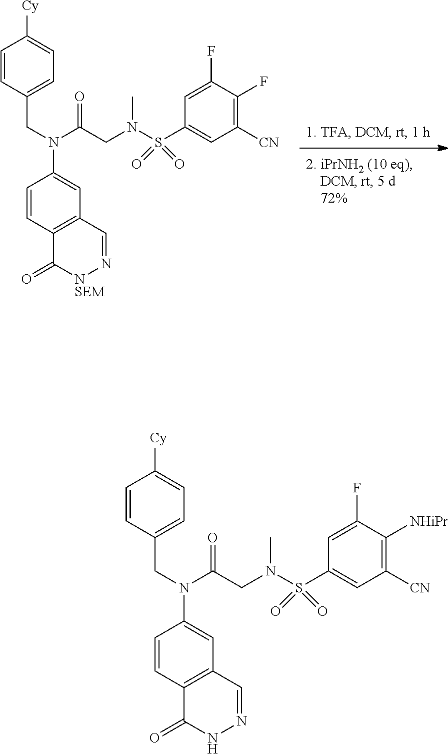

### 2-((3-cyano-5-fluoro-4-(isopropylamino)-N-methylphenyl)sulfonamido)-N-(4-cyclohexylbenzyl)-N-(1-oxo-1,2-dihydrophthalazin-6-yl)acetamide

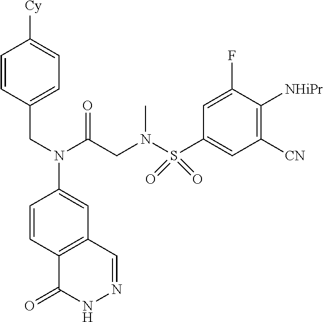

Step 1: To a solution of benzyl (1-oxo-2-((2-(trimethylsilyl)ethoxy)methyl)-1,2-dihydrophthalazin-6-yl)carbamate (500 mg, 1.17 mmol, 1.0 equiv) in 6 mL DMF was added KHMDS (1.4 mL, 1.41 mmol, 1.2 equiv, 1.0 M in THF) at 0° C. dropwise under Argon. 10 minutes late, 1-(bromomethyl)-4-cyclohexylbenzene (387 mg, 1.53 mmol, 1.3 equiv) was added to the reaction mixture. The reaction was allowed to warm up to room temperature and stirred at room temperature for 20 h. Then the reaction was quenched with saturated NH4Cl aq and extracted with ethyl acetate (3×). The combined organic extracts were washed with saturated brine, dried over anhydrous sodium sulfate, and concentrated under reduced pressure. The resulting residue was used directly for next step without further purification.

Step 2: The above residue and Pd/C (90 mg) were dissolved in 10 mL MeOH under hydrogen gas (1 atm). After 24 h, the catalyst was filtered off through a celite pad and washed with ethyl acetate. The combined solvent was concentrated under reduced pressure and the residue was purified through recrystallization (Hex/EtOAc 6/1) to obtain 6-((4-cyclohexylbenzyl)amino)-2-((2-(trimethylsilyl)ethoxy)methyl)phthalazin-1(2H)-one as white solid (300 mg, 55% over 2 steps). 1H NMR (500 MHz, CDCl3) δ 8.20 (d, J=8.7 Hz, 1H), 7.98 (s, 1H), 7.35-7.26 (m, 3H), 7.24-7.20 (m, 1H), 7.13-6.93 (m, 1H), 6.67 (s, 1H), 5.52 (s, 2H), 4.40 (s, 2H), 3.73-3.69 (m, 2H), 2.50 (s, 1H), 1.90-1.72 (m, 5H), 1.49-1.21 (m, 5H), 1.12-0.94 (m, 2H), −0.02 (s, 9H).

Step 3: To a solution of 6-((4-cyclohexylbenzyl)amino)-2-((2-(trimethylsilyl)ethoxy) methyl)phthalazin-1(2H)-one (100 mg, 0.22 mmol, 1.0 equiv) was added MeMgBr (0.2 mL, 0.29 mmol, 1.3 equiv, 1.4 M in THF/toluene) dropwise at 0° C. under Argon. After 10 minutes, the solution of N-((3-cyano-4,5-difluorophenyl)sulfonyl)-N-methylglycinoyl chloride (0.32 mmol, 1.5 equiv) in 1 mL THF was added at 0° C. under Argon. The reaction was allowed to warm up to room temperature and stirred at room temperature for 1 h. Then the reaction was quenched with water, extracted with EtOAc (3×). The combined organic extracts were washed with saturated brine, dried over anhydrous sodium sulfate, and concentrated under reduced pressure. The resulting residue was purified by flash chromatography (eluent: hexane/EtOAc 2.5/1) to provide 24(3-cyano-4,5-difluoro-N-methylphenyl)sulfonamido)-N-(4-cyclohexylbenzyl)-N-(1-oxo-2-((2-(trimethylsilyl)ethoxy) methyl)-1,2-dihydrophthalazin-6-yl)acetamide as light yellow oil (150 mg, 94%). 1H NMR (300 MHz, CDCl3) δ 8.47 (d, J=8.5 Hz, 1H), 8.09 (s, 1H), 8.04-7.86 (m, 2H), 7.47-7.29 (m, 2H), 7.21-7.11 (m, 2H), 6.98 (d, J=7.7 Hz, 2H), 5.56 (s, 2H), 4.82 (s, 2H), 3.90 (s, 2H), 3.74 (t, J=8.3 Hz, 2H), 2.93 (s, 3H), 2.55-2.43 (m, 1H), 1.90-1.70 (m, 5H), 1.52-1.30 (m, 5H), 0.99 (t, J=8.1 Hz, 2H), 0.01 (s, 9H).

Step 4: To a solution of 24(3-cyano-4,5-difluoro-N-methylphenyl) sulfonamido)-N-(4-cyclohexylbenzyl)-N-(1-oxo-2-((2-(trimethylsilyl)ethoxy) methyl)-1,2-dihydrophthalazin-6-yl)acetamide (30 mg, 0.10 mmol, 1.0 equiv) in 1 mL DCM was added 1 mL TFA. The reaction was stirred at room temperature for 1 h. Then the reaction was concentrated under reduced pressure and dried under high vacuum for 1 h. The resulting residue was used directly for next step without further purification.

To a solution of the above residue in 1 mL DCM was added iPrNH2 (67 μL, 0.39 mmol, 2.0 equiv) at 0° C. and stirred at 0° C. for 24 h. Then the reaction was concentrated under reduced pressure. The resulting residue was purified by preparative TLC plates (eluent: DCM/MeOH 10/1) to provide 2-((3-cyano-5-fluoro-4-(isopropylamino)-N-methylphenyl)sulfonamido)-N-(4-cyclohexylbenzyl)-N-(1-oxo-1,2-dihydrophthalazin-6-yl)acetamide as white foam (18 mg, 72%). 1H NMR (300 MHz, CDCl3) δ 9.92 (s, 1H), 8.42 (d, J=8.8 Hz, 1H), 8.06 (s, 1H), 7.66 (s, 1H), 7.56-7.33 (m, 3H), 7.14 (d, J=7.9 Hz, 2H), 7.03 (d, J=7.8 Hz, 2H), 4.88 (s, 2H), 4.68 (s, 1H), 4.52-4.40 (m, 1H), 3.83 (s, 2H), 2.87 (s, 3H), 2.54-2.43 (m, 1H), 1.94-1.68 (m, 5H), 1.46-1.14 (m, 11H). 19F NMR (282 MHz, CDCl3) δ−129.09. LRMS (ESI) m/z 645.3 [M+H]+, 667.3 [M+Na]+; HRMS (ESI) m/z 645.2656 [M+H]+, 667.2473 [M+Na]+; Purity 100%.

### Example 72

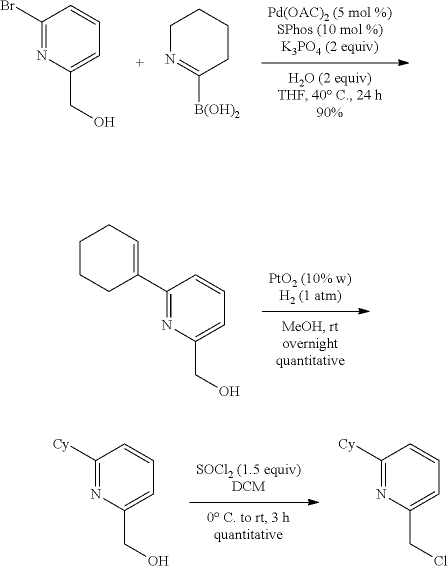

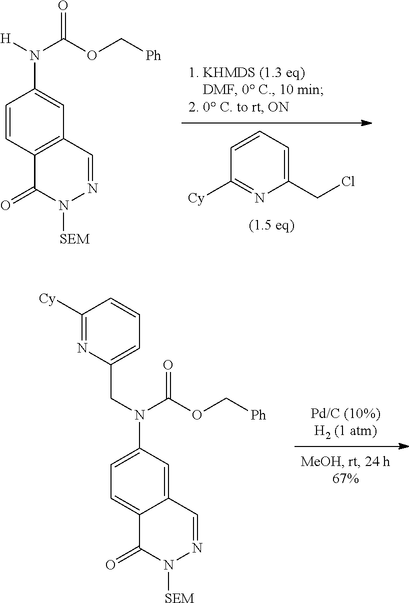

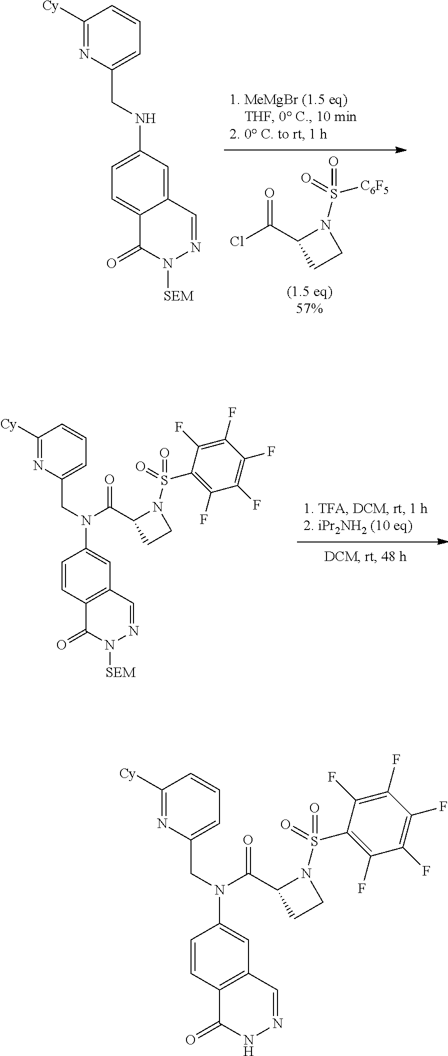

### (R)—N-((6-cyclohexylpyridin-2-yl)methyl)-N-(1-oxo-1,2-dihydrophthalazin-6-yl)-1-((perfluorophenyl)sulfonyl)azetidine-2-carboxamide

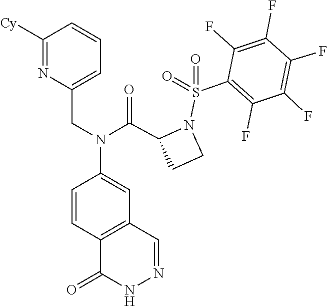

Step 1: (6-bromopyridin-2-yl)methanol (200 mg, 1.06 mmol, 1.0 equiv), cyclohex-1-en-1-ylboronic acid (201 mg, 1.60 mmol, 1.5 equiv), Pd(OAc)2 (12 mg, 0.05 mmol, 5 mol %), SPhos (44 mg, 0.11 mmol, 10 mol %), K3PO4 (452 mg, 2.13 mmol, 2.0 equiv) were dissolved in 5 mL THF and 38 μL H2O under Argon. The reaction was heated at 40° C. for 24 h. Then the reaction was quenched with water and extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 4/1) to provide (6-(cyclohex-1-en-1-yl)pyridin-2-yl)methanol as light yellow oil (180 mg, 90%). 1H NMR (300 MHz, CDCl3) δ 7.62 (t, J=7.8 Hz, 1H), 7.29 (s, 1H), 7.01 (d, J=7.6 Hz, 1H), 6.89-6.74 (m, 1H), 4.73 (s, 2H), 4.30 (s, 1H), 2.73-2.19 (m, 5H), 2.01-1.59 (m, 5H).

Step 2: (6-(cyclohex-1-en-1-yl)pyridin-2-yl)methanol (180 mg) and PtO2 (18 mg) were dissolved in EtOAc/MeOH (8 mL, 1/1) under hydrogen gas (1 atm). After 24 h, the catalyst was filtered off through a celite pad and washed with ethyl acetate. The combined solvent was concentrated under reduced pressure and dried under high vacuum for 1 h. The residue was used directly for next step without further purification. 4(6-cyclohexylpyridin-2-yl)methanol (light yellow oil, 187 mg, quantitative yield).

To the above residue in 5 mL DCM was added SOCl2 (89 μL, 1.22 mmol, 1.3 equiv) dropwise at 0° C. under Argon. Then the reaction was allowed to warm up to room temperature and stirred at room temperature for 3 h. Then the reaction was quenched with cold water and saturated NaHCO3 aqueous solution was added to adjust pH to 7-8. The mixture was extracted with DCM (3×). The combined organic extracts were washed with saturated brine, dried over anhydrous sodium sulfate, concentrated under reduced pressure, and dried under high vacuum for 30 minutes. The residue was used directly for next step without further purification.

To a solution of benzyl (1-oxo-2-((2-(trimethylsilyl)ethoxy)methyl)-1,2-dihydrophthalazin-6-yl)carbamate (307 mg, 0.72 mmol, 1.0 equiv) in 3 mL DMF was added KHMDS (0.94 mL, 0.94 mmol, 1.3 equiv, 1.0 M in THF) at 0° C. dropwise under Argon. 10 minutes late, the solution of the above residue in 1 mL DMF was added to the reaction mixture. The reaction was allowed to warm up to room temperature and stirred at room temperature for 20 h. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 4/1) to provide benzyl ((6-cyclohexylpyridin-2-yl)methyl)(1-oxo-2-((2-(trimethylsilyl)ethoxy)methyl)-1,2-dihydrophthalazin-6-yl)carbamate as light yellow oil (75 mg, 32% brsm). 1H NMR (300 MHz, CDCl3) δ 8.37 (d, J=8.6 Hz, 1H), 8.08 (s, 1H), 7.91-7.80 (m, 2H), 7.55 (t, J=7.7 Hz, 1H), 7.38-7.26 (m, 3H), 7.26-7.13 (m, 2H), 7.06 (dd, J=7.7, 1.8 Hz, 2H), 5.57 (s, 2H), 5.22 (s, 2H), 5.07 (s, 2H), 3.78-3.67 (m, 2H), 2.72-2.60 (m, 1H), 1.97-1.76 (m, 5H), 1.58-1.32 (m, 5H), 1.05-0.94 (m, 2H), −0.00 (s, 9H).

Step 3: benzyl ((6-cyclohexylpyridin-2-yl)methyl)(1-oxo-2-((2-(trimethylsilyl)ethoxy) methyl)-1,2-dihydrophthalazin-6-yl)carbamate (75 mg) and Pd/C (8 mg) were dissolved in 2 mL MeOH under hydrogen gas (1 atm). After 24 h, the catalyst was filtered off through a celite pad and washed with ethyl acetate. The combined solvent was concentrated under reduced pressure and the residue was purified by preparative TLC plates (Hex/EtOAc 2/1) to obtain 6-(((6-cyclohexylpyridin-2-yl)methyl)amino)-2-((2-(trimethylsilyl)ethoxy)methyl)phthalazin-1(2H)-one as white solid (39 mg, 67%). 1H NMR (300 MHz, CDCl3) δ 8.20 (d, J=8.7 Hz, 1H), 7.99 (s, 1H), 7.60 (t, J=7.7 Hz, 1H), 7.16-7.03 (m, 3H), 6.67 (d, J=2.2 Hz, 1H), 5.99 (s, 1H), 5.52 (s, 2H), 4.50 (d, J=4.5 Hz, 2H), 3.79-3.63 (m, 2H), 2.81-2.65 (m, 1H), 2.04-1.73 (m, 5H), 1.62-1.25 (m, 5H), 1.03-0.90 (m, 2H), −0.03 (s, 9H).

Step 4: To a solution of 6-(((6-cyclohexylpyridin-2-yl)methyl)amino)-2-((2-(trimethylsilyl)ethoxy) methyl)phthalazin-1(2H)-one as white solid (35 mg, 0.075 mmol, 1.0 equiv) in 1 mL THF was added MeMgBr (80 μL, 0.11 mmol, 1.5 equiv, 1.4 M in THF/toluene) dropwise at 0° C. under Argon. After 10 minutes, (R)-1-((perfluorophenyl)sulfonyl)azetidine-2-carbonyl chloride (34 mg, 0.098 mmol, 1.3 equiv) was added at 0° C. under Argon. The reaction was allowed to warm up to room temperature and stirred at room temperature for 1 h. Then the reaction was quenched with water and extracted with EtOAc (3×). The combined organic extracts were washed with saturated brine, dried over anhydrous sodium sulfate, and concentrated under reduced pressure. The resulting residue was purified by preparative TLC plates (eluent: hexane/acetone 3/1) to provide (R)—N-((6-cyclohexylpyridin-2-yl)methyl)-N-(1-oxo-2-((2-(trimethylsilyl)ethoxy)methyl)-1,2-dihydrophthalazin-6-yl)-1-((perfluorophenyl)sulfonyl)azetidine-2-carboxamide as light yellow oil (33 mg, 57%). 1H NMR (300 MHz, CDCl3) δ 8.44 (d, J=8.4 Hz, 1H), 8.09 (s, 1H), 7.72 (s, 1H), 7.64 (dd, J=8.4, 1.7 Hz, 1H), 7.57 (t, J=7.7 Hz, 1H), 7.20-6.97 (m, 2H), 5.55 (s, 2H), 5.22-4.78 (m, 3H), 4.21-3.98 (m, 2H), 3.81-3.65 (m, 2H), 2.70-2.53 (m, 1H), 2.41-2.22 (m, 1H), 2.08-1.93 (m, 1H), 1.91-1.68 (m, 5H), 1.53-1.18 (m, 5H), 1.07-0.90 (m, 2H), −0.01 (s, 9H).

Step 5: To a solution of (R)—N-((6-cyclohexylpyridin-2-yl)methyl)-N-(1-oxo-2-((2-(trimethylsilyl)ethoxy)methyl)-1,2-dihydrophthalazin-6-yl)-1-((perfluorophenyl)sulfonyl) azetidine-2-carboxamide (33 mg, 0.04 mmol, 1.0 equiv) in 1 mL DCM was added 1 mL TFA. The reaction was stirred at room temperature for 1 h. Then the reaction was concentrated under reduced pressure and dried under high vacuum for 1 h. The resulting residue was used directly for next step without further purification.

To a solution of the above residue in 1 mL DCM was added iPrNH2 (36 μL, 0.42 mmol, 10.0 equiv) at room temperature and stirred for 48 h. Then the reaction was concentrated under reduced pressure. The resulting residue was purified by preparative TLC plates (eluent: hexane/acetone 7/6) to provide (R)—N-((6-cyclohexylpyridin-2-yl)methyl)-N-(1-oxo-1,2-dihydrophthalazin-6-yl)-1-((perfluorophenyl)sulfonyl)azetidine-2-carboxamide as white solid (5 mg, 19%). 1H NMR (300 MHz, CDCl3) δ 10.15 (s, 1H), 8.41 (d, J=8.2 Hz, 1H), 8.08 (s, 1H), 7.82-7.52 (m, 3H), 7.06 (d, J=7.9 Hz, 2H), 5.03 (s, 2H), 4.96-4.84 (m, 1H), 4.19-3.97 (m, 2H), 3.68-3.52 (m, 1H), 2.41-2.24 (m, 1H), 2.06-1.93 (m, 1H), 1.93-1.69 (m, 5H), 1.54-1.25 (m, 5H). LRMS (ESI) m/z 648.2 [M+H]+; HRMS (ESI) m/z 648.1706 [M+H]+, 670.1518 [M+Na]+; Purity 98%.

### Example 73

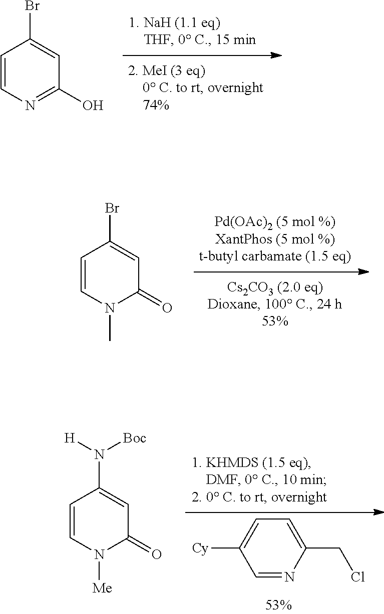

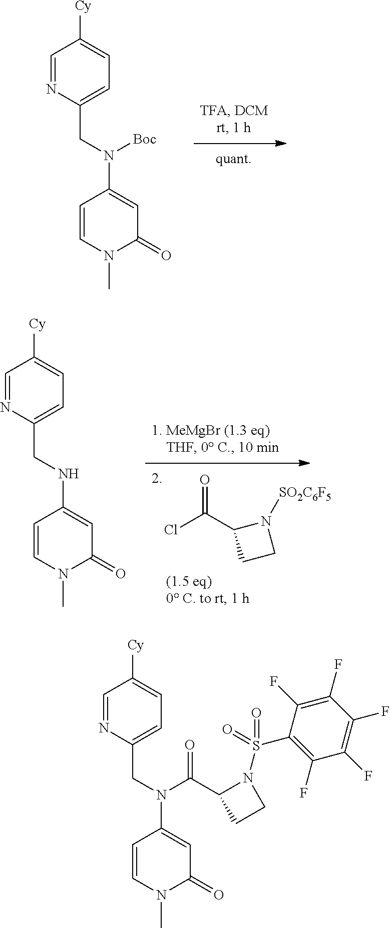

### (R)—N-((5-cyclohexylpyridin-2-yl)methyl)-N-(1-methyl-2-oxo-1,2-dihydropyridin-4-yl)-1-((perfluorophenyl)sulfonyl)azetidine-2-carboxamide

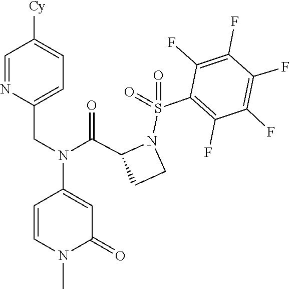

Step 1: To a solution of 4-bromopyridin-2-ol (1.0 g, 5.75 mmol, 1.0 equiv) in 25 mL THF was added NaH (253 mg, 6.33 mmol, 1.1 equiv, 60% in mineral oil) slowly at 0° C. under Argon. After 15 minutes, CH3I (1.1 mL, 17.3 mmol, 3.0 equiv) was added at 0° C. Then the reaction was allowed to warm up to room temperature and stirred for overnight. Then the reaction was quenched with saturated NH4Cl aqueous solution and extracted with ethyl acetate (3×). The combined organic extracts were washed with saturated brine, dried over anhydrous sodium sulfate, and concentrated under reduced pressure to obtain 4-bromo-1-methylpyridin-2(1H)-one as brown solid (800 mg, 74%). 1H NMR (300 MHz, CDCl3) δ 7.14 (d, J=7.2 Hz, 1H), 6.82 (d, J=2.0 Hz, 1H), 6.32 (dd, J=7.0, 2.0 Hz, 1H), 3.50 (s, 3H).

Step 2: 4-bromo-1-methylpyridin-2(1H)-one (500 mg, 2.67 mmol, 1.0 equiv), tert-butyl carbamate (467 mg, 3.99 mmol, 1.5 equiv), and Cs2CO3 (1.73 g, 5.32 mmol, 2.0 equiv) were dissolved in 15 mL 1,4-dioxane. After 10 minutes, Pd(OAc)2 (30 mg, 0.13 mmol, 5 mol %) and XantPhos (80 mg, 0.13 mmol, 5 mol %) were added to the reaction under Argon. Then the reaction was heated at 100° C. for 24 h. The reaction was quenched with water, extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone/MeOH 1/1/4%) to provide tert-butyl (1-methyl-2-oxo-1,2-dihydropyridin-4-yl)carbamate as white solid (315 mg, 53%). 1H NMR (300 MHz, CDCl3) δ 7.19 (d, J=7.4 Hz, 1H), 6.76-6.62 (m, 2H), 6.28 (s, 1H), 3.49 (s, 3H), 1.50 (s, 9H).

Step 3: To a solution of tert-butyl (1-methyl-2-oxo-1,2-dihydropyridin-4-yl)carbamate (310 mg, 1.38 mmol, 1.0 equiv) in 12 mL DMF was added KHMDS (2.1 mL, 2.10 mmol, 1.5 equiv, 1.0 M in THF) at 0° C. dropwise under Argon. 10 minutes late, 2-(chloromethyl)-5-cyclohexylpyridine (4.2 mL, 2.10 mmol, 1.5 equiv, 0.5 M in toluene) was added to the reaction mixture. The reaction was allowed to warm up to room temperature and stirred at room temperature for 24 h. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone 2/1) to provide tert-butyl ((5-cyclohexylpyridin-2-yl)methyl)(1-methyl-2-oxo-1,2-dihydropyridin-4-yl)carbamate as red foam (293 mg, 53%). 1H NMR (300 MHz, CDCl3) δ 8.39 (d, J=2.0 Hz, 1H), 7.46 (dd, J=8.1, 2.2 Hz, 1H), 7.14 (d, J=7.6 Hz, 1H), 7.07 (d, J=8.0 Hz, 1H), 6.60 (dd, J=7.5, 2.5 Hz, 1H), 6.27 (d, J=2.5 Hz, 1H), 4.91 (s, 2H), 3.47 (s, 3H), 2.59-2.45 (m, 1H), 1.99-1.76 (m, 6H), 1.59-1.30 (m, 13H).

Step 4: To a solution of tert-butyl ((5-cyclohexylpyridin-2-yl)methyl)(1-methyl-2-oxo-1,2-dihydropyridin-4-yl)carbamate (293 mg) in 6 mL DCM was added TFA (3 mL). The reaction was stirred at room temperature for 1 h. Then the reaction was concentrated under reduced pressure, then quenched with saturated NaHCO3 aqueous solution to pH 8. The reaction was extracted with DCM (3×). The combined organic extracts were washed with saturated brine, dried over anhydrous sodium sulfate, and concentrated under reduced pressure. The resulting residue was dried under high vacuum to obtain crude product 4-(((5-cyclohexylpyridin-2-yl)methyl)amino)-1-methylpyridin-2(1H)-one as light yellow foam (220 mg, quantitative yield), which was used directly for next step without further purification. 1H NMR (300 MHz, CDCl3) δ 8.42 (d, J=2.0 Hz, 1H), 7.52 (dd, J=8.0, 2.2 Hz, 1H), 7.21 (d, J=8.0 Hz, 1H), 7.03 (d, J=7.4 Hz, 1H), 5.75 (ddd, J=7.3, 2.5, 0.6 Hz, 1H), 5.59 (d, J=2.4 Hz, 1H), 5.49 (s, 1H), 4.36 (d, J=4.8 Hz, 2H), 3.44 (s, 3H), 2.61-2.48 (m, 1H), 1.97-1.80 (m, 5H), 1.53-1.32 (m, 5H).

Step 5: To a solution of 4-(((5-cyclohexylpyridin-2-yl)methyl)amino)-1-methylpyridin-2(1H)-one (80 mg, 0.27 mmol, 1.0 equiv) in 3 mL THF was added MeMgBr (0.25 mL, 0.35 mmol, 1.3 equiv, 1.4 M in THF/toluene) dropwise at 0° C. under Argon. After 10 minutes, (R)-1-((perfluorophenyl)sulfonyl)azetidine-2-carbonyl chloride (141 mg, 0.40 mmol, 1.5 equiv) was added at 0° C. under Argon. The reaction was allowed to warm up to room temperature and stirred at room temperature for 1 h. Then the reaction was quenched with water and extracted with EtOAc (3×). The combined organic extracts were washed with saturated brine, dried over anhydrous sodium sulfate, and concentrated under reduced pressure. The resulting residue was purified by preparative TLC plates (eluent: hexane/acetone 6/7) to provide (R)—N-((5-cyclohexylpyridin-2-yl)methyl)-N-(1-methyl-2-oxo-1,2-dihydropyridin-4-yl)-1-((perfluorophenyl)sulfonyl)azetidine-2-carboxamide as white foam (20 mg, 12%). 1H NMR (300 MHz, CDCl3) δ 8.32 (d, J=1.4 Hz, 1H), 7.48 (dd, J=8.0, 2.0 Hz, 1H), 7.32 (d, J=7.2 Hz, 1H), 7.13 (d, J=8.0 Hz, 1H), 6.26 (d, J=2.1 Hz, 1H), 6.21 (dd, J=7.2, 1.9 Hz, 1H), 5.21 (t, J=7.7 Hz, 1H), 4.84 (q, J=15.7 Hz, 2H), 4.25-4.15 (m, 1H), 4.13-4.04 (m, 1H), 3.52 (s, 3H), 2.57-2.44 (m, 1H), 2.44-2.28 (m, 2H), 1.92-1.75 (m, 6H), 1.50-1.28 (m, 4H). LRMS (ESI) m/z 611.3 [M+H]+; HRMS (ESI) m/z 611.1756 [M+H]+, 633.1568 [M+Na]+; Purity 100%.

### Example 74

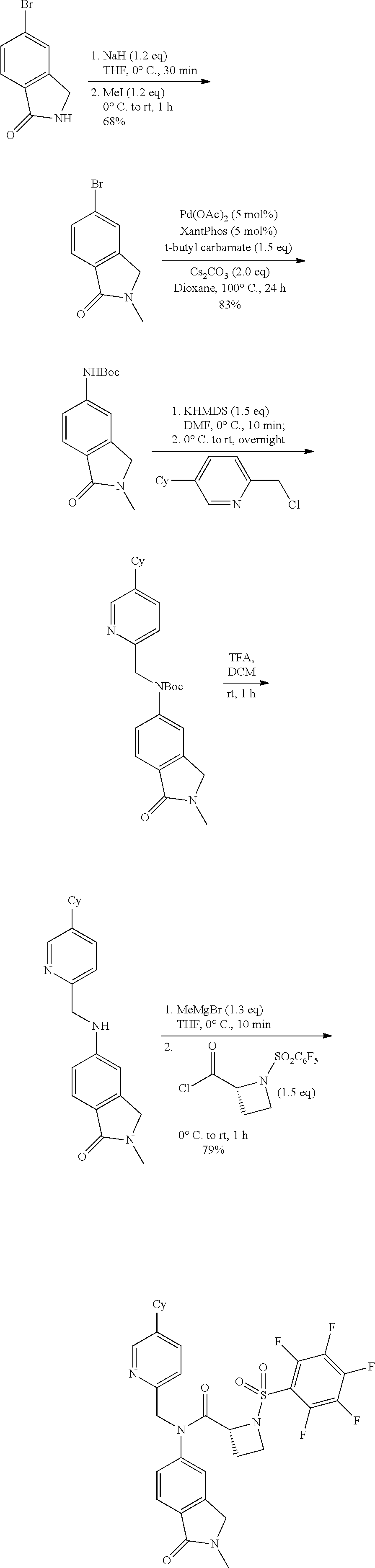

### (R)—N-((5-cyclohexylpyridin-2-yl)methyl)-N-(2-methyl-1-oxoisoindolin-5-yl)-1-((perfluorophenyl)sulfonyl)azetidine-2-carboxamide

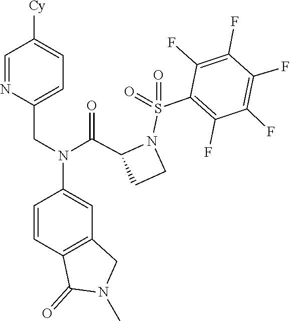

Step 1: To a solution of 5-bromoisoindolin-1-one (1.2 g, 5.66 mmol, 1.0 equiv) in 30 mL DMF was added NaH (272 mg, 6.79 mmol, 1.2 equiv, 60% in mineral oil) slowly at 0° C. under Argon. After 30 minutes, CH3I (0.42 mL, 6.79 mmol, 1.2 equiv) was added at 0° C. Then the reaction was allowed to warm up to room temperature and stirred for 1 h. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: DCM/acetone 10/1) to obtain 5-bromo-2-methylisoindolin-1-one as light yellow solid (870 mg, 68%). 1H NMR (300 MHz, CDCl3) δ 7.77-7.66 (m, 1H), 7.67-7.54 (m, 2H), 4.35 (s, 2H), 3.18 (s, 3H).

Step 2: 5-bromo-2-methylisoindolin-1-one (800 mg, 3.54 mmol, 1.0 equiv), tert-butyl carbamate (621 mg, 5.31 mmol, 1.5 equiv), and Cs2CO3 (2.31 g, 7.08 mmol, 2.0 equiv) were dissolved in 20 mL 1,4-dioxane. After 10 minutes, Pd(OAc)2 (40 mg, 0.18 mmol, 5 mol %) and XantPhos (104 mg, 0.18 mmol, 5 mol %) were added to the reaction under Argon. Then the reaction was heated at 100° C. for 24 h. The reaction was quenched with water, extracted with ethyl acetate (3×). The combined organic extracts were washed with saturated brine, dried over anhydrous sodium sulfate, and concentrated under reduced pressure. The resulting residue was purified by flash chromatography (eluent: DCM/acetone 5/1/) to provide tert-butyl (2-methyl-1-oxoisoindolin-5-yl)carbamate as white solid (767 mg, 83%).

Step 3: To a solution of tert-butyl (2-methyl-1-oxoisoindolin-5-yl)carbamate (500 mg, 1.91 mmol, 1.0 equiv) in 10 mL DMF was added KHMDS (2.5 mL, 2.48 mmol, 1.5 equiv, 1.0 M in THF) at 0° C. dropwise under Argon. 10 minutes late, 2-(chloromethyl)-5-cyclohexylpyridine (5.8 mL, 2.10 mmol, 1.5 equiv, 0.5 M in toluene) was added to the reaction mixture. The reaction was allowed to warm up to room temperature and stirred at room temperature for 24 h. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone 2/1) to provide tert-butyl ((5-cyclohexylpyridin-2-yl)methyl)(2-methyl-1-oxoisoindolin-5-yl)carbamate (583 mg) with less polar impurities as mixture, which was used directly for next step without further purification.

To a solution of the above residue (580 mg) in 12 mL DCM was added TFA (6 mL). The reaction was stirred at room temperature for 1 h. Then the reaction was concentrated under reduced pressure, then quenched with saturated NaHCO3 aqueous solution to pH 8. The reaction was extracted with DCM (3×). The combined organic extracts were washed with saturated brine, dried over anhydrous sodium sulfate, and concentrated under reduced pressure. The resulting residue was purified by flash chromatography (eluent: hexane/acetone 1/2) to provide 5-(((5-cyclohexylpyridin-2-yl)methyl)amino)-2-methylisoindolin-1-one (290 mg) with less polar impurities as mixture, which was further purified through recrystallization (hexane/acetone 2/1, 5 mL) to obtain pure product as yellow solid (137 mg, 21% over 2 steps). 1H NMR (300 MHz, CDCl3) δ 8.44 (d, J=2.2 Hz, 1H), 7.66-7.54 (m, 2H), 7.30 (d, J=8.2 Hz, 1H), 7.27 (d, J=1.8 Hz, 1H), 6.70 (dd, J=8.3, 2.1 Hz, 1H), 6.64 (s, 1H), 4.51 (s, 2H), 4.24 (s, 2H), 3.12 (s, 3H), 2.62-2.51 (m, 1H), 1.99-1.83 (m, 5H), 1.48-1.31 (m, 5H).

Step 4: To a solution of 5-(((5-cyclohexylpyridin-2-yl)methyl)amino)-2-methylisoindolin-1-one (20 mg, 0.06 mmol, 1.0 equiv) in 1 mL THF was added MeMgBr (55 μL, 0.08 mmol, 1.3 equiv, 1.4 M in THF/toluene) dropwise at 0° C. under Argon. After 10 minutes, (R)-1-((perfluorophenyl)sulfonyl)azetidine-2-carbonyl chloride (31 mg, 0.09 mmol, 1.5 equiv) was added at 0° C. under Argon. The reaction was allowed to warm up to room temperature and stirred at room temperature for 1 h. Then the reaction was quenched with water and extracted with EtOAc (3×). The combined organic extracts were washed with saturated brine, dried over anhydrous sodium sulfate, and concentrated under reduced pressure. The resulting residue was purified by flash chromatography (eluent: hexane/acetone 1.5/1) to provide (R)—N-((5-cyclohexylpyridin-2-yl)methyl)-N-(2-methyl-1-oxoisoindolin-5-yl)-1-((perfluorophenyl)sulfonyl) azetidine-2-carboxamide as yellow foam (30 mg, 79%). 1H NMR (300 MHz, CDCl3) δ 8.33 (s, 1H), 7.82 (d, J=7.9 Hz, 1H), 7.53 (d, J=8.1 Hz, 1H), 7.36 (s, 1H), 7.27-7.14 (m, 2H), 5.17-4.85 (m, 3H), 4.39 (s, 2H), 4.23-4.02 (m, 2H), 3.22 (s, 3H), 2.62-2.44 (m, 1H), 2.43-2.27 (m, 1H), 2.14-1.80 (m, 6H), 1.52-1.27 (m, 5H). LRMS (ESI) m/z 649.2 [M+H]+; HRMS (ESI) m/z 649.1937 [M+H]+; Purity 100%.

### Example 75

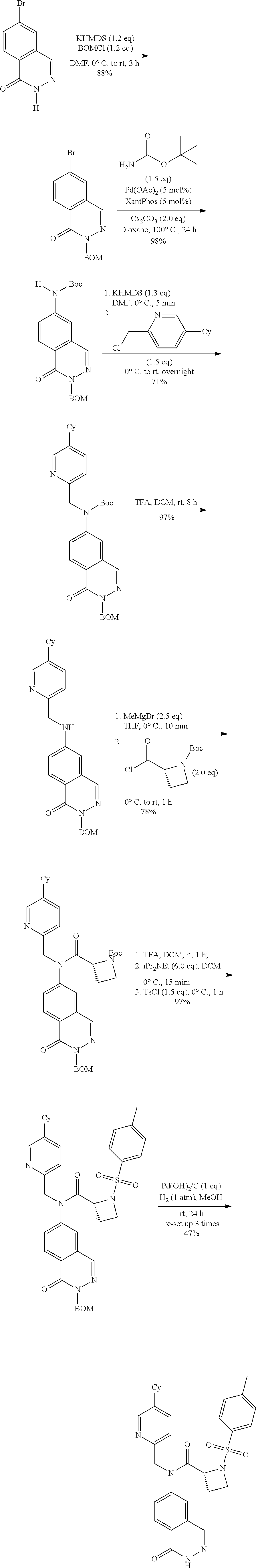

### (R)—N-((5-cyclohexylpyridin-2-yl)methyl)-N-(1-oxo-1,2-dihydrophthalazin-6-yl)-1-tosylazetidine-2-carboxamide

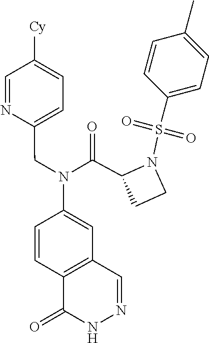

Step 1: To a solution of 6-bromophthalazin-1(2H)-one (612 mg, 2.72 mmol, 1.0 equiv) in 14 mL DMF was added KHMDS (3.3 mL, 3.26 mmol, 1.2 equiv, 1.0 M in THF) at 0° C. under Argon. After 10 minutes, BOMCl (3.3 mmol, 1.2 equiv) was added to the reaction mixture dropwise. The reaction was allowed to warm up to room temperature and stirred at room temperature for 3 h. Then the reaction was quenched with cold saturated NH4Cl aqueous solution and extracted with EtOAc (3×). The combined organic extracts were washed with saturated brine, dried over anhydrous sodium sulfate, and concentrated under reduced pressure. The resulting residue was purified by flash chromatography (eluent: hexane/EtOAc 3/1) to give 2-((benzyloxy)methyl)-6-bromophthalazin-1(2H)-one as white solid (836 mg, 88%). 1H NMR (300 MHz, CDCl3) δ 8.30 (d, J=8.4 Hz, 1H), 8.10 (s, 1H), 7.93-7.81 (m, 2H), 7.40-7.24 (m, 5H), 5.64 (s, 2H), 4.74 (s, 2H).

Step 2: 2-((benzyloxy)methyl)-6-bromophthalazin-1(2H)-one (814 mg, 2.36 mmol, 1.0 equiv), tert-butyl carbamate (415 mg, 3.55 mmol, 1.5 equiv), and Cs2CO3 (1.54 g, 4.72 mmol, 2.0 equiv) were dissolved in 30 mL 1,4-dioxane. After 10 minutes, Pd(OAc)2 (27 mg, 0.12 mmol, 5 mol %) and XantPhos (68 mg, 0.12 mmol, 5 mol %) were added to the reaction under Argon. Then the reaction was heated at 100° C. for 24 h. The reaction was quenched with water, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 2/3) to provide tert-butyl (2-((benzyloxy)methyl)-1-oxo-1,2-dihydrophthalazin-6-yl)carbamate as white solid (883 mg, 98%). 1H NMR (300 MHz, CDCl3) δ 8.33 (d, J=8.7 Hz, 1H), 8.12 (s, 1H), 8.05 (d, J=2.0 Hz, 1H), 7.44 (dd, J=8.7, 2.2 Hz, 1H), 7.40-7.35 (m, 2H), 7.33-7.23 (m, 3H), 6.94 (s, 1H), 5.64 (s, 2H), 4.74 (s, 2H), 1.55 (s, 9H).

Step 3: To a solution of tert-butyl (2-((benzyloxy)methyl)-1-oxo-1,2-dihydrophthalazin-6-yl)carbamate (305 mg, 0.80 mmol, 1.0 equiv) in 6 mL DMF was added KHMDS (1.0 mL, 1.04 mmol, 1.3 equiv, 1.0 M in THF) at 0° C. dropwise under Argon. 10 minutes later, 2-(chloromethyl)-5-cyclohexylpyridine (2.1 mL, 2.08 mmol, 1.3 equiv, 0.5 M in toluene) was added to the reaction mixture. The reaction was allowed to warm up to room temperature and stirred at room temperature for 20 h. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 11/9) to provide tert-butyl (2-((benzyloxy)methyl)-1-oxo-1,2-dihydrophthalazin-6-yl)((5-cyclohexyl pyridin-2-yl)methyl)carbamate as colorless oil (314 mg, 71%). 1H NMR (300 MHz, CDCl3) δ 8.44 (d, J=2.1 Hz, 1H), 8.36 (d, J=8.7 Hz, 1H), 8.11 (s, 1H), 7.79 (dd, J=8.7, 2.1 Hz, 1H), 7.73 (d, J=2.0 Hz, 1H), 7.54 (dd, J=8.1, 2.2 Hz, 1H), 7.42-7.21 (m, 6H), 5.65 (s, 2H), 5.06 (s, 2H), 4.74 (s, 2H), 2.62-2.50 (m, 1H), 1.96-1.74 (m, 6H), 1.51-1.34 (m, 13H).

Step 4: To a solution of tert-butyl (2-((benzyloxy)methyl)-1-oxo-1,2-dihydrophthalazin-6-yl)((5-cyclohexyl pyridin-2-yl)methyl)carbamate (308 mg, 0.56 mmol) in 9 mL DCM was added TFA (3 mL). The reaction was stirred at room temperature for 8 h. Then the reaction was concentrated under reduced pressure, then quenched with saturated NaHCO3 aqueous solution to pH 8. The reaction was extracted with ethyl acetate (3×). The combined organic extracts were washed with saturated brine, dried over anhydrous sodium sulfate, and concentrated under reduced pressure. The resulting residue was dried under high vacuum to provide 2-((benzyloxy)methyl)-6-(((5-cyclohexylpyridin-2-yl)methyl)amino)phthalazin-1(2H)-one as brown solid (245 mg, 97%), which was used directly for next step.

Step 5: To a solution of 2-((benzyloxy)methyl)-6-(((5-cyclohexylpyridin-2-yl)methyl)amino)phthalazin-1(2H)-one (200 mg, 0.44 mmol, 1.0 equiv) in 5 mL THF was added MeMgBr (0.79 mL, 1.10 mmol, 2.5 equiv, 1.4 M in THF/toluene) at 0° C. under Argon. 10 minutes later, a solution of tert-butyl (R)-2-(chlorocarbonyl)azetidine-1-carboxylate (0.88 mmol, 2.0 equiv) in 3 mL THF was added to the reaction. The reaction was allowed to warm up to room temperature and stirred for 1 h. Then the reaction was quenched with saturated NH4Cl aqueous solution, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 1/1) to provide tert-butyl (R)-2-((2-((benzyloxy)methyl)-1-oxo-1,2-dihydrophthalazin-6-yl)((5-cyclohexylpyridin-2-yl)methyl)carbamoyl)azetidine-1-carboxylate as light yellow solid (226 mg, 78%). 1H NMR (300 MHz, CDCl3) δ 8.44 (d, J=8.3 Hz, 1H), 8.35 (s, 1H), 8.13 (s, 1H), 7.82-7.63 (m, 2H), 7.55 (d, J=6.4 Hz, 1H), 7.48 (s, 1H), 7.42-7.35 (m, 2H), 7.35-7.29 (m, 2H), 7.27-7.21 (m, 1H), 5.66 (s, 2H), 5.13 (s, 2H), 4.75 (s, 2H), 4.70-4.57 (m, 1H), 4.13-4.06 (m, 1H), 3.85-3.73 (m, 1H), 2.58-2.47 (m, 1H), 2.31-2.14 (m, 2H), 1.96-1.68 (m, 6H), 1.57-1.33 (m, 13H).

Step 6: To a solution of tert-butyl (R)-2-((2-((benzyloxy)methyl)-1-oxo-1,2-dihydrophthalazin-6-yl)((5-cyclohexylpyridin-2-yl)methyl)carbamoyl)azetidine-1-carboxylate (96 mg, 0.15 mmol) in 2 mL DCM was added TFA (1 mL). The reaction was stirred at room temperature for 1 h. Then the reaction was concentrated under reduced pressure, diluted with dry DCE and concentrated again. The residue was dried under high vacuum for 30 minutes and used directly for the next step.

To a solution of the above residue in 3 mL DCM was added DIPEA (150 μL, 0.90 mmol, 6.0 equiv) at 0° C. under Argon. After 10 minutes, TsCl (44 mg, 0.23 mmol, 1.5 equiv) was added under Argon at 0° C. The reaction was stirred at 0° C. for 1 h. Then the reaction was quenched with saturated NH4Cl aqueous solution, and extracted with DCM (3×). The combined organic extracts were washed with saturated brine, dried over anhydrous sodium sulfate, and concentrated under reduced pressure. The resulting residue was purified by flash chromatography (eluent: DCM/MeOH 50/1) to provide (R)—N-(2-((benzyloxy)methyl)-1-oxo-1,2-dihydrophthalazin-6-yl)-N-((5-cyclohexylpyridin-2-yl)methyl)-1-tosylazetidine-2-carboxamide as white foam (100 mg, 97%). 1H NMR (300 MHz, CDCl3) δ 12.01 (s, 1H), 8.39 (s, 1H), 8.29 (d, J=8.4 Hz, 1H), 8.01 (s, 1H), 7.75-7.32 (m, 6H), 7.21 (d, J=7.9 Hz, 2H), 5.26-4.98 (m, 2H), 4.63 (s, 1H), 3.78-3.53 (m, 2H), 2.61-2.47 (m, 1H), 2.44-2.13 (m, 5H), 1.96-1.63 (m, 6H), 1.51-1.29 (m, 4H). LRMS (ESI) m/z 572.3 [M+H]+; HRMS (ESI) m/z 572.2340 [M+H]+, 594.2155 [M+Na]+; Purity 100%.

### Example 76

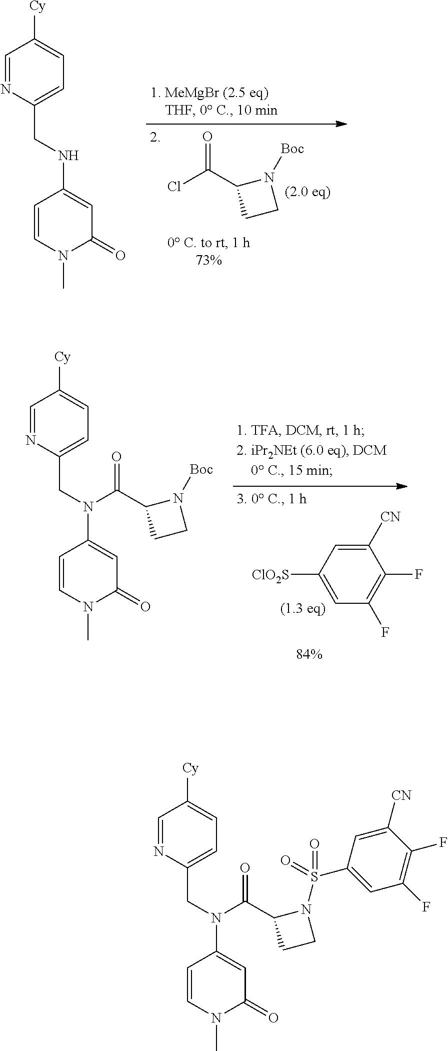

### (R)-1-((3-cyano-4,5-difluorophenyl)sulfonyl)-N-((5-cyclohexylpyridin-2-yl)methyl)-N-(1-methyl-2-oxo-1,2-dihydropyridin-4-yl)azetidine-2-carboxamide

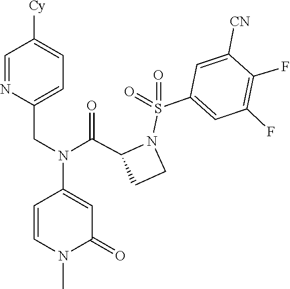

Step 1: To a solution of 4-(((5-cyclohexylpyridin-2-yl)methyl)amino)-1-methylpyridin-2(1H)-one (125 mg, 0.42 mmol, 1.0 equiv) in 5 mL THF was added MeMgBr (0.75 mL, 1.05 mmol, 2.5 equiv, 1.4 M in THF/toluene) at 0° C. under Argon. 10 minutes later, a solution of tert-butyl (R)-2-(chlorocarbonyl)azetidine-1-carboxylate (0.84 mmol, 2.0 equiv) in 3 mL THF was added to the reaction. The reaction was allowed to warm up to room temperature and stirred for 1 h. Then the reaction was quenched with saturated NH4Cl aqueous solution, extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone/MeOH 1/4/4%) to provide tert-butyl (R)-2-(((5-cyclohexylpyridin-2-yl)methyl)(1-methyl-2-oxo-1,2-dihydropyridin-4-yl)carbamoyl)azetidine-1-carboxylate as white foam (148 mg, 73%). 1H NMR (300 MHz, CDCl3) δ 8.32 (d, J=1.8 Hz, 1H), 7.55-7.42 (m, 1H), 7.41-7.26 (m, 2H), 6.51-6.21 (m, 2H), 5.18-4.73 (m, 3H), 4.22-4.03 (m, 1H), 3.93-3.71 (m, 1H), 3.51 (s, 3H), 2.59-2.42 (m, 1H), 2.43-2.28 (m, 1H), 2.27-2.18 (m, 1H), 1.99-1.68 (m, 6H), 1.57-1.26 (m, 11H).

Step 2: To a solution of tert-butyl (R)-2-(((5-cyclohexylpyridin-2-yl)methyl)(1-methyl-2-oxo-1,2-dihydropyridin-4-yl)carbamoyl)azetidine-1-carboxylate (143 mg, 0.30 mmol) in 5 mL DCM was added TFA (1.5 mL). The reaction was stirred at room temperature for 1 h. Then the reaction was concentrated under reduced pressure, diluted with dry DCE and concentrated again. The residue was dried under high vacuum for 30 minutes and used directly for the next step.

To a solution of the above residue in 4 mL DCM was added DIPEA (0.3 mL, 1.79 mmol, 6.0 equiv) at 0° C. under Argon. After 10 minutes, 3-cyano-4,5-difluorobenzenesulfonyl chloride (92 mg, 0.39 mmol, 1.3 equiv) was added dropwise under Argon at 0° C. The reaction was stirred at 0° C. for 1 h. Then the reaction was quenched with saturated NH4Cl aqueous solution, and extracted with DCM (3×). The combined organic extracts were washed with saturated brine, dried over anhydrous sodium sulfate, and concentrated under reduced pressure. The resulting residue was purified by flash chromatography (eluent: hexane/acetone/MeOH 1/4/4%) to provide (R)-1-((3-cyano-4,5-difluorophenyl)sulfonyl)-N-((5-cyclohexylpyridin-2-yl)methyl)-N-(1-methyl-2-oxo-1,2-dihydropyridin-4-yl)azetidine-2-carboxamide as white foam (146 mg, 84%). 1H NMR (300 MHz, CDCl3) δ 8.35 (d, J=1.3 Hz, 1H), 8.10 (ddd, J=9.1, 7.1, 2.0 Hz, 1H), 8.05-7.98 (m, 1H), 7.50 (dd, J=8.1, 2.0 Hz, 1H), 7.35 (d, J=7.4 Hz, 1H), 7.21 (d, J=8.1 Hz, 1H), 6.34-6.21 (m, 2H), 5.18 (t, J=8.0 Hz, 1H), 4.90 (q, J=17.6 Hz, 2H), 4.14-3.99 (m, 1H), 3.77-3.63 (m, 1H), 3.53 (s, 3H), 2.58-2.36 (m, 2H), 2.33-2.21 (m, 1H), 1.91-1.73 (m, 6H), 1.49-1.30 (m, 4H). LRMS (ESI) m/z 582.2 [M+H]+; HRMS (ESI) m/z 582.1985 [M+H]+, 604.1805 [M+Na]+; Purity 100%.

### Example 77

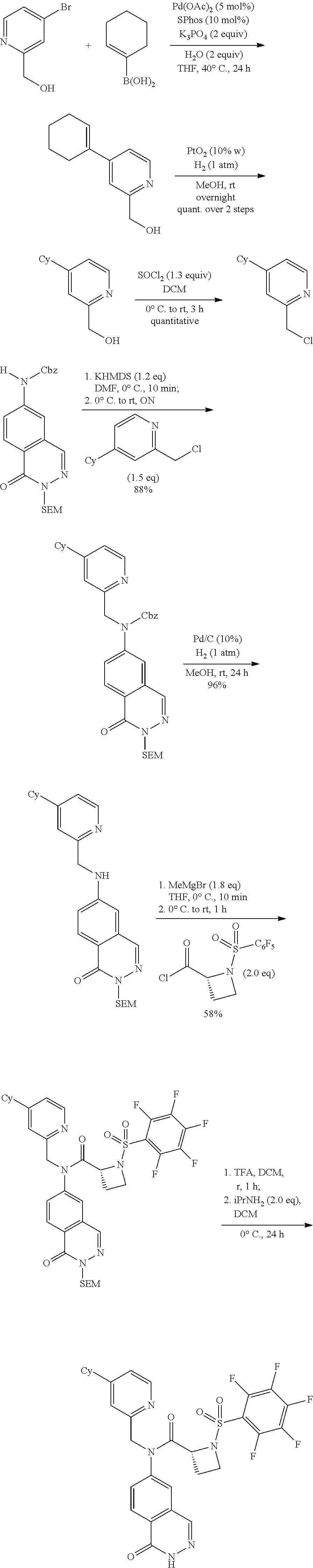

### (R)—N-((4-cyclohexylpyridin-2-yl)methyl)-N-(1-oxo-1,2-dihydrophthalazin-6-yl)-1-((perfluorophenyl)sulfonyl)azetidine-2-carboxamide

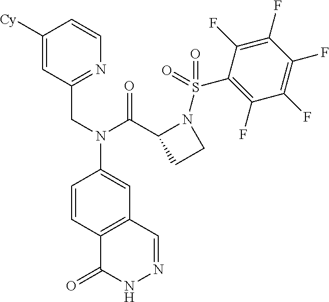

Preparation by a similar procedure to Example 72, except with (4-bromopyridin-2-yl)methanol as starting material in step 1.

Step 2: (4-cyclohexylpyridin-2-yl)methanol (quantitative yield over 2 steps, dark red oil). 1H NMR (300 MHz, CDCl3) δ 8.41 (d, J=5.0 Hz, 1H), 7.10 (s, 1H), 7.06 (d, J=5.1 Hz, 1H), 4.72 (s, 2H), 3.58 (s, 1H), 2.57-2.43 (m, 1H), 1.97-1.64 (m, 6H), 1.49-1.34 (m, 4H).

Step 3: Benzyl ((4-cyclohexylpyridin-2-yl)methyl)(1-oxo-2-((2-(trimethylsilyl)ethoxy) methyl)-1,2-dihydrophthalazin-6-yl)carbamate as light yellow oil (247 mg, 88%). 1H NMR (300 MHz, CDCl3) δ 8.42 (dd, J=5.1, 0.7 Hz, 1H), 8.39-8.30 (m, 1H), 8.07 (s, 1H), 7.81-7.67 (m, 2H), 7.36-7.19 (m, 5H), 7.13-7.01 (m, 2H), 5.54 (s, 2H), 5.22 (s, 2H), 5.12 (s, 2H), 3.77-3.65 (m, 2H), 2.47-2.37 (m, 1H), 1.91-1.70 (m, 5H), 1.46-1.26 (m, 5H), 1.03-0.92 (m, 2H), −0.02 (s, 9H).

Step 4: 6-(((4-cyclohexylpyridin-2-yl)methyl)amino)-2-((2-(trimethylsilyl)ethoxy) methyl)phthalazin-1(2H)-one as grey foam (178 mg, 96%). 1H NMR (300 MHz, CDCl3) δ 8.47 (d, J=5.1 Hz, 1H), 8.21 (d, J=8.8 Hz, 1H), 8.00 (s, 1H), 7.15 (s, 1H), 7.12-7.04 (m, 2H), 6.65 (d, J=2.2 Hz, 1H), 5.81 (s, 1H), 5.52 (s, 2H), 4.51 (s, 2H), 3.75-3.67 (m, 2H), 2.55-2.47 (m, 1H), 1.90-1.75 (m, 6H), 1.45-1.33 (m, 4H), 1.01-0.92 (m, 2H), −0.02 (s, 9H).

Step 5: (R)—N-((4-cyclohexylpyridin-2-yl)methyl)-N-(1-oxo-2-((2-(trimethylsilyl) ethoxy)methyl)-1,2-dihydrophthalazin-6-yl)-1-((perfluorophenyesulfonyl) azetidine-2-carboxamide as white foam (172 mg, 58%). 1H NMR (300 MHz, CDCl3) δ 8.51-8.31 (m, 2H), 8.18-8.09 (m, 1H), 7.68-7.51 (m, 2H), 7.22-7.03 (m, 2H), 5.55 (s, 2H), 5.16-4.89 (m, 3H), 4.20-3.97 (m, 2H), 3.85-3.63 (m, 2H), 2.58-2.46 (m, 1H), 2.42-2.26 (m, 1H), 1.84-1.72 (m, 7H), 1.50-1.32 (m, 4H), 1.06-0.94 (m, 2H), −0.01 (s, 9H).

Step 6: (R)—N-((4-cyclohexylpyridin-2-yl)methyl)-N-(1-oxo-1,2-dihydrophthalazin-6-yl)-1-((perfluorophenyl)sulfonyl)azetidine-2-carboxamide as white foam (75 mg, 53%). 1H NMR (300 MHz, CDCl3) δ 11.21 (s, 1H), 8.52-8.22 (m, 2H), 8.02 (s, 1H), 7.58 (s, 1H), 7.52-7.40 (m, 1H), 7.26-7.20 (m, 1H), 7.09 (d, J=4.8 Hz, 1H), 5.26-4.83 (m, 3H), 4.25-3.91 (m, 2H), 2.59-2.42 (m, 1H), 2.40-2.22 (m, 1H), 2.04-1.69 (m, 7H), 1.53-1.28 (m, 4H). LRMS (ESI) m/z 648.3 [M+H]+; HRMS (ESI) m/z 648.1705 [M+H]+, 670.1522 [M+Na]+; Purity 100%.

### Example 78

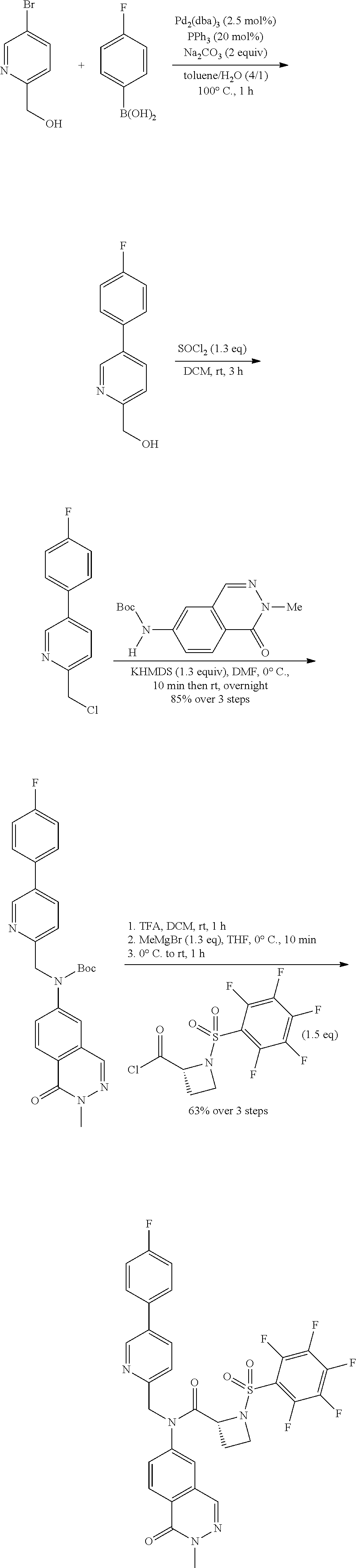

### (R)—N-((5-(4-fluorophenyl)pyridin-2-yl)methyl)-N-(2-methyl-1-oxo-1,2-dihydrophthalazin-6-yl)-1-((perfluorophenyl)sulfonyl)azetidine-2-carboxamide

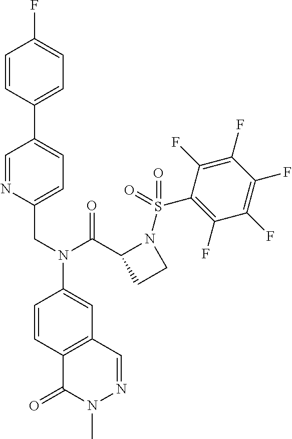 [SMILES: Cn1ncc2cc(N(Cc3ccc(-c4ccc(F)cc4)cn3)C(=O)[C@H]3CCN3S(=O)(=O)c3c(F)c(F)c(F)c(F)c3F)ccc2c1=O]

Step 1: (5-bromopyridin-2-yl)methanol (100 mg, 0.53 mmol, 1.0 equiv), (4-fluorophenyl)boronic acid (111 mg, 0.80 mmol, 1.5 equiv), Pd2(dba)3 (12 mg, 0.01 mmol, 2.5 mol %), PPh3 (28 mg, 0.11 mmol, 20 mol %), Na2CO3 (113 mg, 1.06 mmol, 2.0 equiv) were dissolved in 2 mL toluene and 0.5 mL water under Argon. The reaction was heated at 100° C. for 1 h. Then the reaction was quenched with water and extracted with ethyl acetate (3×). The combined organic extracts were washed with 25% NaOH aqueous solution, saturated brine, dried over anhydrous sodium sulfate, and concentrated under reduced pressure. The resulting residue was used directly for next step.

Step 2: To the above residue in 5 mL DCM was added SOCl2 (47 μL, 0.65 mmol, 1.3 equiv) dropwise at 0° C. under Argon. Then the reaction was allowed to warm up to room temperature and stirred at room temperature for 3 h. Then the reaction was quenched with cold water and saturated NaHCO3 aqueous solution was added to adjust pH to 7-8. The mixture was extracted with DCM (3×). The combined organic extracts were washed with saturated brine, dried over anhydrous sodium sulfate, concentrated under reduced pressure, and dried under high vacuum for 30 minutes. The residue was used directly for next step without further purification.

Step 3: To a solution of tert-butyl (2-methyl-1-oxo-1,2-dihydrophthalazin-6-yl)carbamate (69 mg, 0.25 mmol, 1.0 equiv) in 3 mL DMF was added KHMDS (0.3 mL, 0.33 mmol, 1.3 equiv, 1.0 M in THF) at 0° C. dropwise under Argon. 10 minutes late, the solution of the above residue in 2 mL toluene was added to the reaction mixture. The reaction was allowed to warm up to room temperature and stirred at room temperature overnight. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone 5/1) to provide tert-butyl ((5-(4-fluorophenyl)pyridin-2-yl)methyl)(2-methyl-1-oxo-1,2-dihydrophthalazin-6-yl)carbamate as white foam (98 mg, 85% over 3 steps). 1H NMR (300 MHz, CDCl3) δ 8.76 (dd, J=2.2, 0.5 Hz, 1H), 8.34 (d, J=8.7 Hz, 1H), 8.06 (d, J=0.4 Hz, 1H), 7.85 (dd, J=8.1, 2.4 Hz, 1H), 7.77 (dd, J=8.7, 2.2 Hz, 1H), 7.72 (d, J=2.0 Hz, 1H), 7.58-7.51 (m, 2H), 7.38 (d, J=8.2 Hz, 1H), 7.21-7.12 (m, 2H), 5.11 (s, 2H), 3.83 (s, 3H), 1.44 (s, 9H).

Step 4: To a solution of tert-butyl ((5-(4-fluorophenyl)pyridin-2-yl)methyl)(2-methyl-1-oxo-1,2-dihydrophthalazin-6-yl)carbamate as white foam (98 mg) in 3 mL DCM was added 1 mL TFA The reaction was stirred at room temperature for 1 h. Then the reaction was concentrated under reduced pressure, diluted with dry DCE and concentrated again. The residue was dried under high vacuum for 30 minutes and used directly for the next step.

Step 5: To a solution of the above residue in 2 mL THF was added MeMgBr (0.2 mL, 0.28 mmol, 1.3 equiv, 1.4 M in THF/toluene) dropwise at 0° C. under Argon. After 10 minutes, (R)-1-((perfluorophenyl)sulfonyl)azetidine-2-carbonyl chloride (112 mg, 0.32 mmol, 1.5 equiv) was added at 0° C. under Argon. The reaction was allowed to warm up to room temperature and stirred at room temperature for 1 h. Then the reaction was quenched with water and extracted with EtOAc (3×). The combined organic extracts were washed with saturated brine, dried over anhydrous sodium sulfate, and concentrated under reduced pressure. The resulting residue was purified by flash chromatography (eluent: hexane/acetone 3/1) to provide (R)—N-((5-(4-fluorophenyl)pyridin-2-yl)methyl)-N-(2-methyl-1-oxo-1,2-dihydrophthalazin-6-yl)-1-((perfluorophenyesulfonyl)azetidine-2-carboxamide as light yellow foam (90 mg, 63% over 3 steps). 1H NMR (300 MHz, CDCl3) δ 8.67 (s, 1H), 8.44 (d, J=8.4 Hz, 1H), 8.11 (s, 1H), 7.88-7.79 (m, 1H), 7.66 (s, 1H), 7.61 (d, J=8.5 Hz, 1H), 7.56-7.47 (m, 2H), 7.37 (d, J=7.9 Hz, 1H), 7.22-7.11 (m, 2H), 5.15-4.92 (m, 3H), 4.15-4.02 (m, 2H), 3.84 (s, 3H), 2.40-2.28 (m, 1H), 2.02-1.89 (m, 1H). LRMS (ESI) m/z 674.2 [M+H]+; HRMS (ESI) m/z 674.1296 [M+H]+, 696.1110 [M+Na]+; Purity 100%.

### Example 79

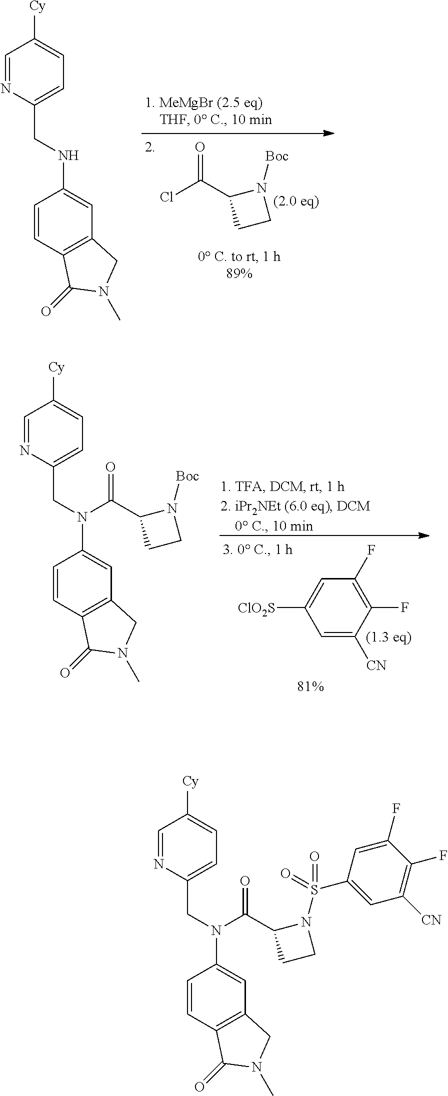

### (R)-1-((3-cyano-4,5-difluorophenyl)sulfonyl)-N-((5-cyclohexylpyridin-2-yl)methyl)-N-(2-methyl-1-oxoisoindolin-5-yl)azetidine-2-carboxamide

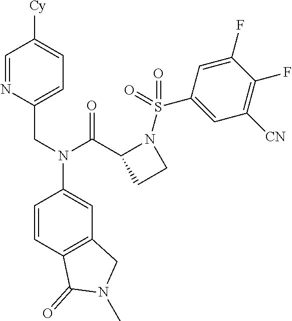

Step 1: To a solution of 5-(((5-cyclohexylpyridin-2-yl)methyl)amino)-2-methylisoindolin-1-one (100 mg, 0.30 mmol, 1.0 equiv) in 3 mL THF was added MeMgBr (0.5 mL, 0.75 mmol, 2.5 equiv, 1.4 M in THF/toluene) at 0° C. under Argon. 10 minutes later, a solution of tert-butyl (R)-2-(chlorocarbonyl)azetidine-1-carboxylate (0.60 mmol, 2.0 equiv) in 3 mL THF was added to the reaction. The reaction was allowed to warm up to room temperature and stirred for 1 h. Then the reaction was quenched with saturated NH4Cl aqueous solution, extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone 1/1) to provide tert-butyl (R)-2-(((5-cyclohexylpyridin-2-yl)methyl)(2-methyl-1-oxoisoindolin-5-yl)carbamoyl)azetidine-1-carboxylate as light yellow foam (138 mg, 89%). 1H NMR (300 MHz, CDCl3) δ 8.30 (s, 1H), 7.78 (d, J=7.8 Hz, 1H), 7.64-7.26 (m, 4H), 5.06 (s, 2H), 4.66-4.49 (m, 1H), 4.35 (s, 2H), 4.15-4.01 (m, 1H), 3.85-3.69 (m, 1H), 3.19 (s, 3H), 2.58-2.43 (m, 1H), 2.21-2.06 (m, 2H), 2.00-1.56 (m, 9H), 1.56-1.30 (m, 10H).

Step 2: To a solution of tert-butyl (R)-2-(((5-cyclohexylpyridin-2-yl)methyl)(2-methyl-1-oxoisoindolin-5-yl)carbamoyl)azetidine-1-carboxylate (133 mg, 0.26 mmol) in 5 mL DCM was added TFA (1.5 mL). The reaction was stirred at room temperature for 1 h. Then the reaction was concentrated under reduced pressure, diluted with dry DCE and concentrated again. The residue was dried under high vacuum for 30 minutes and used directly for the next step.

Step 3: To a solution of the above residue in 4 mL DCM was added DIPEA (0.25 mL, 1.54 mmol, 6.0 equiv) at 0° C. under Argon. After 10 minutes, 3-cyano-4,5-difluorobenzenesulfonyl chloride (79 mg, 0.39 mmol, 1.3 equiv) was added dropwise under Argon at 0° C. The reaction was stirred at 0° C. for 1 h. Then the reaction was quenched with saturated NH4Cl aqueous solution, and extracted with DCM (3×). The combined organic extracts were washed with saturated brine, dried over anhydrous sodium sulfate, and concentrated under reduced pressure. The resulting residue was purified by flash chromatography (eluent: hexane/acetone 1/2) to provide (R)-1-((3-cyano-4,5-difluorophenyl)sulfonyl)-N-((5-cyclohexylpyridin-2-yl)methyl)-N-(2-methyl-1-oxoisoindolin-5-yl)azetidine-2-carboxamide as light yellow foam (128 mg, 81%). 1H NMR (300 MHz, CDCl3) δ 8.32 (d, J=1.7 Hz, 1H), 8.16 (ddd, J=9.0, 7.0, 2.1 Hz, 1H), 8.10-8.01 (m, 1H), 7.79 (d, J=8.0 Hz, 1H), 7.49 (dd, J=8.0, 2.2 Hz, 1H), 7.36 (s, 1H), 7.26-7.17 (m, 2H), 5.07-4.86 (m, 3H), 4.45-4.28 (m, 2H), 4.03-3.91 (m, 1H), 3.67-3.57 (m, 1H), 3.18 (s, 3H), 2.55-2.43 (m, 1H), 2.38-2.17 (m, 2H), 1.90-1.69 (m, 6H), 1.46-1.31 (m, 4H).

LRMS (ESI) m/z 620.4 [M+H]+; HRMS (ESI) m/z 620.2146 [M+H]+, 642.1962 [M+Na]+; Purity 99%.

### Example 80

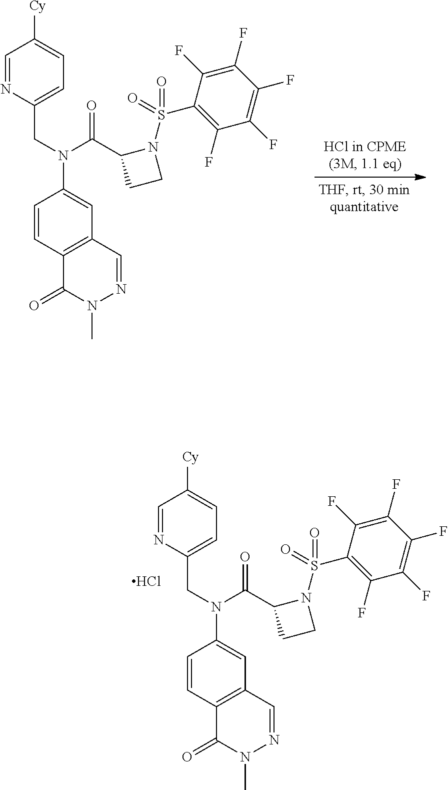

### (R)—N-((5-cyclohexylpyridin-2-yl)methyl)-N-(2-methyl-1-oxo-1,2-dihydrophthalazin-6-yl)-1-((perfluorophenyl)sulfonyl)azetidine-2-carboxamide hydrochloride

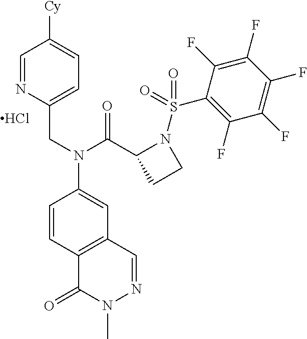

Step 1: To a solution of (R)—N-((5-cyclohexylpyridin-2-yl)methyl)-N-(2-methyl-1-oxo-1,2-dihydrophthalazin-6-yl)-1-((perfluorophenyl)sulfonyl)azetidine-2-carboxamide (120 mg, 0.18 mmol, 1.0 equiv) in 2 mL THF was added HCl (66.5 μL, 0.20 mmol, 1.1 equiv, 3 M in CPME) at room temperature under Argon. 30 minutes later, the solvent was removed under reduced pressure. The residue was dried under high vacuum overnight to provide (R)—N-((5-cyclohexylpyridin-2-yl)methyl)-N-(2-methyl-1-oxo-1,2-dihydrophthalazin-6-yl)-1-((perfluorophenyl)sulfonyl)azetidine-2-carboxamide hydrochloride as white solid (120 mg, quantitative yield). 1H NMR (300 MHz, CDCl3) δ 8.44 (s, broad, 2H), 8.17 (s, broad, 2H), 7.97-7.71 (m, 2H), 7.61 (s, broad, 1H), 5.69-5.26 (m, 2H), 5.01 (s, broad, 1H), 4.18-4.03 (m, 2H), 3.84 (s, 3H), 2.69 (s, broad, 1H), 2.48 (s, broad, 1H), 2.17-1.73 (m, 7H), 1.55-1.31 (m, 4H). HRMS (ESI) m/z 662.1865 [M+H]+, 684.1678 [M+Na]+; Purity 100%.

### Example 81

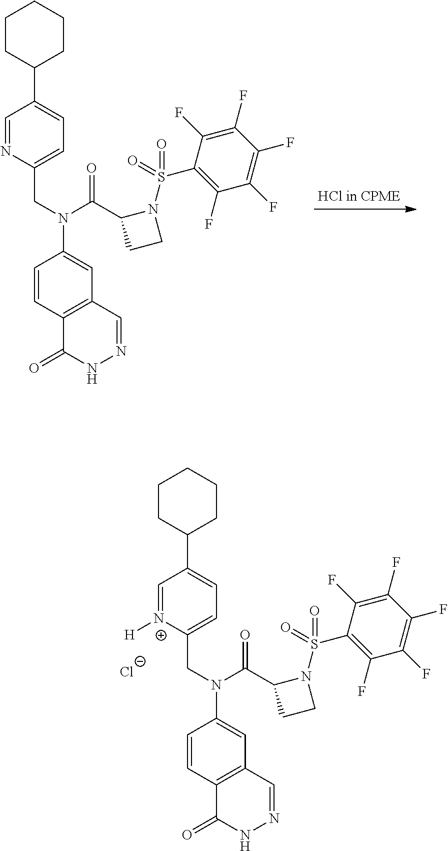 [SMILES: O=C([C@H]1CCN1S(=O)(=O)c1c(F)c(F)c(F)c(F)c1F)N(Cc1ccc(C2CCCCC2)cn1)c1ccc2c(=O)[nH]ncc2c1 ; [Cl-] ; O=C([C@H]1CCN1S(=O)(=O)c1c(F)c(F)c(F)c(F)c1F)N(Cc1ccc(C2CCCCC2)c[nH+]1)c1ccc2c(=O)[nH]ncc2c1]

### (R)-5-cyclohexyl-2-((N-(1-oxo-1,2-dihydrophthalazin-6-yl)-1-((perfluorophenyl)sulfonyl)azetidine-2-carboxamido)methyl)pyridin-1-ium chloride

Step 1. To a solution of (R)—N-((5-cyclohexylpyridin-2-yl)methyl)-N-(1-oxo-1,2-dihydrophthalazin-6-yl)-1-((perfluorophenyl)sulfonyl)azetidine-2-carboxamide (81.9 mg, 0.126 mmol) in THF (1.5 mL) was added a 3M HCl in CPME solution (0.046 mL, 0.139 mmol). The mixture was stirred for 30 minutes, and then concentrated to dryness (84 mg, 100% pure by HPLC). HRMS (ESI) m/z 648.1714 [M+H]+, 670.1528 [M+Na]+.

### Example 82

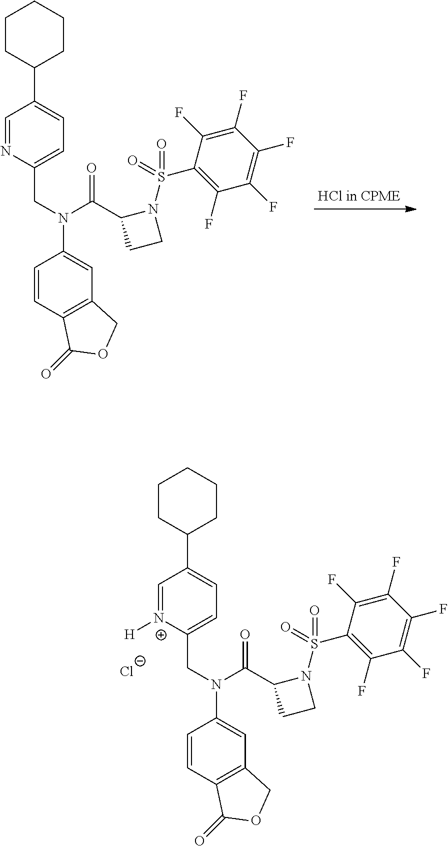 [SMILES: O=C1OCc2cc(N(Cc3ccc(C4CCCCC4)cn3)C(=O)[C@H]3CCN3S(=O)(=O)c3c(F)c(F)c(F)c(F)c3F)ccc21 ; [Cl-] ; O=C1OCc2cc(N(Cc3ccc(C4CCCCC4)c[nH+]3)C(=O)[C@H]3CCN3S(=O)(=O)c3c(F)c(F)c(F)c(F)c3F)ccc21]

### (R)-5-cyclohexyl-2-4N-(1-oxo-1,3-dihydroisobenzofuran-5-yl)-1-((perfluorophenyl)sulfonyl)azetidine-2-carboxamido)methyl)pyridin-1-ium chloride

Step 1. Preparation by a similar procedure to Example 82, step 1, starting from (R)—N-((5-cyclohexylpyridin-2-yl)methyl)-N-(1-oxo-1,3-dihydroisobenzofuran-5-yl)-1-((perfluorophenyl)sulfonyl)azetidine-2-carboxamide (113 mg, 0.178 mmol) to obtain (R)-5-cyclohexyl-2-((N-(1-oxo-1,3-dihydroisobenzofuran-5-yl)-1-((perfluorophenyl)sulfonyl) azetidine-2-carboxamido)methyl)pyridin-1-ium chloride (101 mg, 100% by HPLC). HRMS (ESI) m/z 636.1593 [M+H]+, 658. 1404 [M+Na]+.

### Example 83

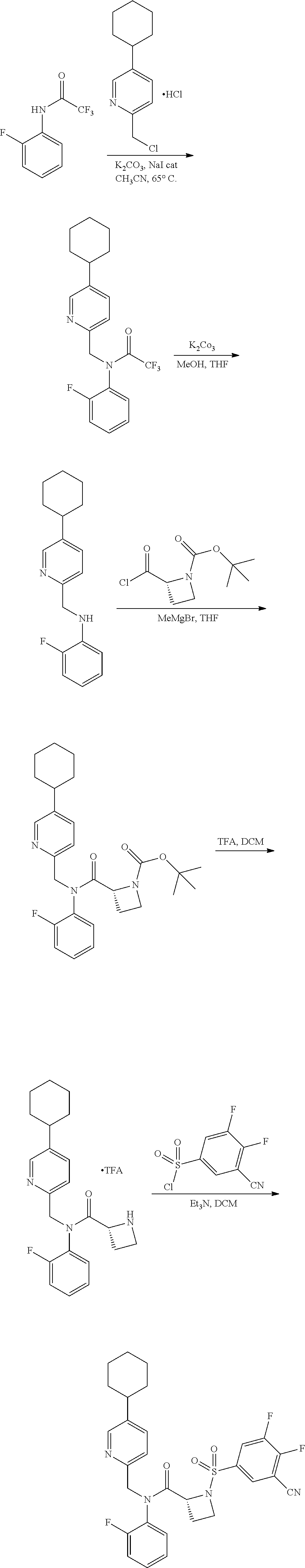

### (R)-1-((3-cyano-4,5-difluorophenyl)sulfonyl)-N-((5-cyclohexylpyridin-2-yl)methyl)-N-(2-fluorophenyl)azetidine-2-carboxamide

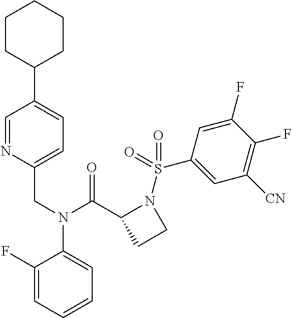 [SMILES: N#Cc1cc(S(=O)(=O)N2CC[C@@H]2C(=O)N(Cc2ccc(C3CCCCC3)cn2)c2ccccc2F)cc(F)c1F]

Step 1. Preparation by a similar procedure to Example 5, step 2, starting from 2,2,2-trifluoro-N-(2-fluorophenyl)acetamide (187 mg, 0.90 mmol) and 2-(chloromethyl)-5-cyclohexylpyridine hydrochloride (280 mg, 1.13 mmol) to obtain N-((5-cyclohexylpyridin-2-yl)methyl)-2,2,2-trifluoro-N-(2-fluorophenyl)acetamide (285 mg, 83% yield).

Step 2. Preparation by a similar procedure to Example 6, step 2, starting from N-((5-cyclohexylpyridin-2-yl)methyl)-2,2,2-trifluoro-N-(2-fluorophenyl)acetamide (285 mg, 0.75 mmol) to obtain N-((5-cyclohexylpyridin-2-yl)methyl)-2-fluoroaniline (153 mg, 72% yield).

Step 3. Preparation by a similar procedure to Example 38, step 4, starting from N-((5-cyclohexylpyridin-2-yl)methyl)-2-fluoroaniline (250 mg, 0.88 mmol) to obtain tert-butyl (R)-2-(((5-cyclohexylpyridin-2-yl)methyl)(2-fluorophenyl)carbamoyl)azetidine-1-carboxylate (170 mg, 41% yield).

Step 4. Preparation by a similar procedure to Example 38, step 5, starting from tert-butyl (R)-2-(((5-cyclohexylpyridin-2-yl)methyl)(2-fluorophenyl)carbamoyl)azetidine-1-carboxylat e (170 mg, 0.36 mmol) to obtain (R)—N-((5-cyclohexylpyridin-2-yl)methyl)-N-(2-fluorophenyl)azetidine-2-carboxamide (155 mg, 92% yield).

Step 5. Preparation by a similar procedure to Example 38, step 6, starting from (R)—N-((5-cyclohexylpyridin-2-yl)methyl)-N-(2-fluorophenyl)azetidine-2-carboxamide (155 mg, 0.33 mmol) to obtain (R)-1-((3-cyano-4,5-difluorophenyl)sulfonyl)-N-((5-cyclohexylpyridin-2-yl)methyl)-N-(2-fluorophenyl)azetidine-2-carboxamide (148 mg, 78% yield). LRMS (ESI) m/z 569.3 [M+H]+

### Example 84

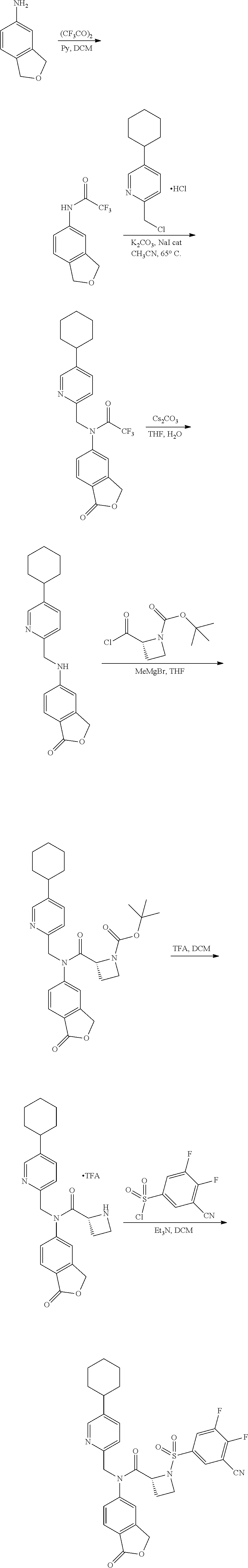

### (R)-1-((3-cyano-4,5-difluorophenyl)sulfonyl)-N-((5-cyclohexylpyridin-2-yl)methyl)-N-(1-oxo-1,3-dihydroisobenzofuran-5-yl)azetidine-2-carboxamide

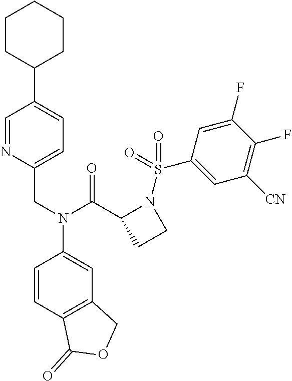 [SMILES: N#Cc1cc(S(=O)(=O)N2CC[C@@H]2C(=O)N(Cc2ccc(C3CCCCC3)cn2)c2ccc3c(c2)COC3=O)cc(F)c1F]

Step 1. Preparation by a similar procedure to Example 5, step 1, starting from 1,3-dihydroisobenzofuran-5-amine (147 mg, 1.09 mmol) to obtain N-(1,3-dihydroisobenzofuran-5-yl)-2,2,2-trifluoroacetamide (206 mg, 82% yield).

Step 2. Preparation by a similar procedure to Example 5, step 2, starting from N-(1,3-dihydroisobenzofuran-5-yl)-2,2,2-trifluoroacetamide (240 mg, 1.04 mmol) to obtain N-((5-cyclohexylpyridin-2-yl)methyl)-2,2,2-trifluoro-N-(1-oxo-1,3-dihydroisobenzofuran-5-yl)acetamide (313 mg, 72% yield).

Step 3. THF (10 mL) and water (3 mL) were added to a flask containing N-((5-cyclohexylpyridin-2-yl)methyl)-2,2,2-trifluoro-N-(1-oxo-1,3-dihydroisobenzofuran-5-yl)acetamide (370 mg, 0.88 mmol) and cesium carbonate (864 mg, 2.65 mmol) under argon. The mixture was stirred at room temperature for 18 h. After the reaction was completed, water was added, and the mixture was extracted with ethyl acetate (2×). The extract was washed with brine, dried (Na2SO4). The crude product was purified by column chromatography to give 5-(((5-cyclohexylpyridin-2-yl)methyl)amino)isobenzofuran-1(3H)-one (566 mg, 85% yield).

Step 4. Preparation by a similar procedure to Example 38, step 4, starting from 5-(((5-cyclohexylpyridin-2-yl)methyl)amino)isobenzofuran-1(3H)-one (250 mg, 0.77 mmol) to obtain tert-butyl (R)-2-(((5-cyclohexylpyridin-2-yl)methyl)(1-oxo-1,3-dihydroisobenzofuran-5-yl)carbamoyl) azetidine-1-carboxylate (125 mg, 32% yield).

Step 5. Preparation by a similar procedure to Example 38, step 5, starting from tert-butyl (R)-2-(((5-cyclohexylpyridin-2-yl)methyl)(1-oxo-1,3-dihydroisobenzofuran-5-yl)carbamoyl) azetidine-1-carboxylate (120 mg, 0.23 mmol) to obtain (R)—N-((5-cyclohexylpyridin-2-yl)methyl)-N-(1-oxo-1,3-dihydroisobenzofuran-5-yl)azetidine-2-carboxamide (143 mg as nTFA salt).

Step 6. Preparation by a similar procedure to Example 38, step 6, starting from (R)—N-((5-cyclohexylpyridin-2-yl)methyl)-N-(1-oxo-1,3-dihydroisobenzofuran-5-yl)azetidine-2-carboxamide (143 mg of nTFA salt) to obtain (R)-1-((3-cyano-4,5-difluorophenyl)sulfonyl)-N-((5-cyclohexylpyridin-2-yl)methyl)-N-(1-oxo-1,3-dihydroisobenzofuran-5-yl)azetidine-2-carboxamide (60 mg, 42% yield for two steps). LRMS (ESI) m/z 607.2 [M+H]+

### Example 85

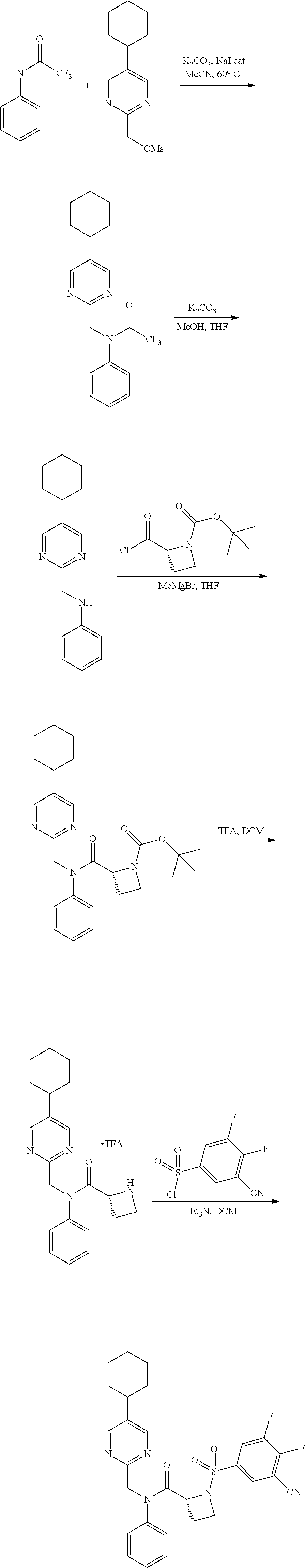

### (R)-1-((3-cyano-4,5-difluorophenyl)sulfonyl)-N-((5-cyclohexylpyrimidin-2-yl)methyl)-N-phenylazetidine-2-carboxamide

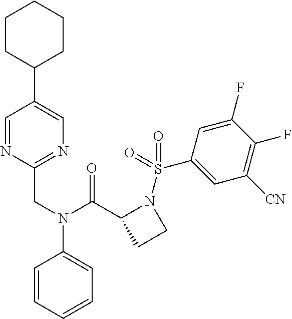 [SMILES: N#Cc1cc(S(=O)(=O)N2CC[C@@H]2C(=O)N(Cc2ncc(C3CCCCC3)cn2)c2ccccc2)cc(F)c1F]

Step 1. Preparation by a similar procedure to Example 38, step 2, starting from 2,2,2-trifluoro-N-phenylacetamide (300 mg, 1.59 mmol) and (5-cyclohexylpyrimidin-2-yl)methyl methanesulfonate (514 mg, 1.90 mmol) to obtain N-((5-cyclohexylpyrimidin-2-yl)methyl)-2,2,2-trifluoro-N-phenylacetamide (144 mg, 25% yield).

Step 2. Preparation by a similar procedure to Example 6, step 2, starting from N-((5-cyclohexylpyrimidin-2-yl)methyl)-2,2,2-trifluoro-N-phenylacetamide (140 mg, 0.38 mmol) to obtain N-((5-cyclohexylpyrimidin-2-yl)methyl)aniline (82 mg, 80% yield).

Step 3. Preparation by a similar procedure to Example 38, step 4, starting from N-((5-cyclohexylpyrimidin-2-yl)methyl)aniline (70 mg, 0.26 mmol) to obtain tert-butyl (R)-2-(((5-cyclohexylpyrimidin-2-yl)methyl)(phenyl)carbamoyl)azetidine-1-carboxylate (46 mg, 39%).

Step 4. Preparation by a similar procedure to Example 38, step 5, starting from tert-butyl (R)-2-(((5-cyclohexylpyrimidin-2-yl)methyl)(phenyl)carbamoyl)azetidine-1-carboxylate (58 mg, 0.13 mmol) to obtain (R)—N-((5-cyclohexylpyrimidin-2-yl)methyl)-N-phenylazetidine-2-carboxamide TFA salt (72 mg) as a crude thick oil carried as such to next step.

Step 5. Preparation by a similar procedure to Example 38, step 6, starting from (R)—N-((5-cyclohexylpyrimidin-2-yl)methyl)-N-phenylazetidine-2-carboxamide TFA salt (65 mg of crude thick oil from last step) to obtain (R)-1-((3-cyano-4,5-difluorophenyl)sulfonyl)-N-((5-cyclohexylpyrimidin-2-yl)methyl)-N-phenylazetidine-2-carboxamide (20 mg, 28% yield for 2 steps). LRMS (ESI) m/z 552.2 [M+H]+.

### Example 86

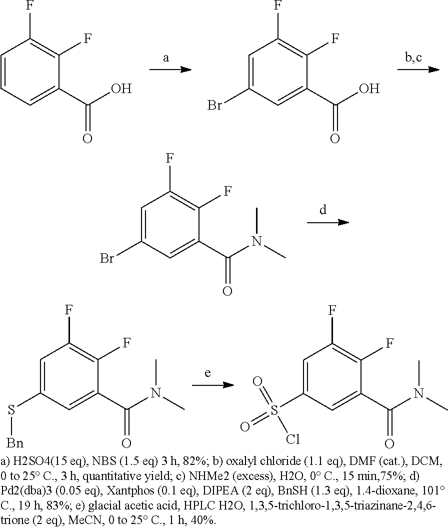 [SMILES: O=C(O)c1cccc(F)c1F ; O=C(O)c1cc(Br)cc(F)c1F ; CN(C)C(=O)c1cc(Br)cc(F)c1F ; CN(C)C(=O)c1cc(SCc2ccccc2)cc(F)c1F ; CN(C)C(=O)c1cc(S(=O)(=O)Cl)cc(F)c1F]

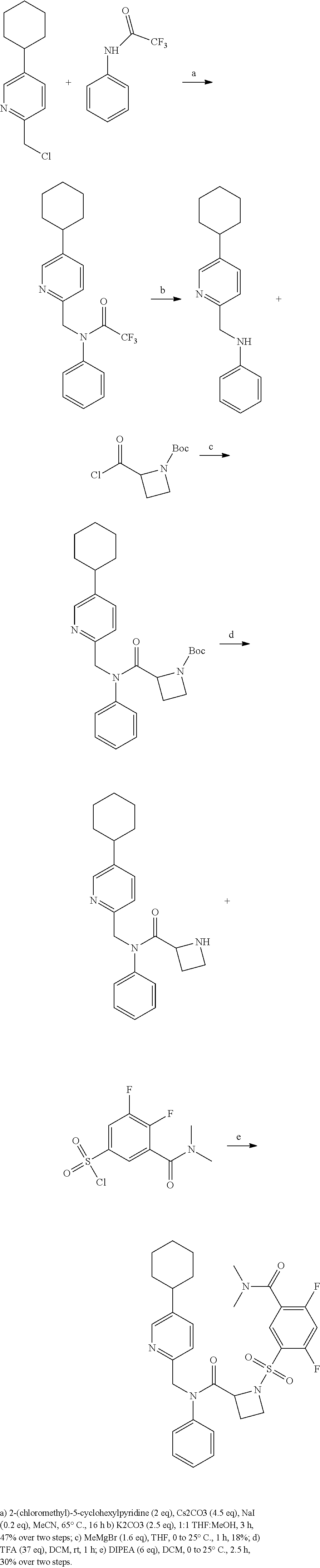

(R)—N-((5-cyclohexylpyridin-2-yl)methyl)-1-((3-(dimethylcarbamoyl)-4,5-difluorophenyl)sulfonyl)-N-phenylazetidine-2-carboxamide

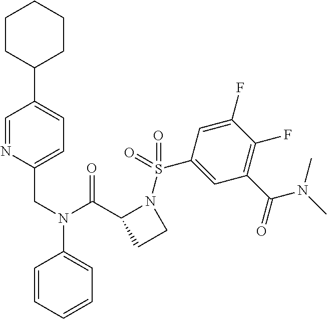 [SMILES: CN(C)C(=O)c1cc(S(=O)(=O)N2CC[C@@H]2C(=O)N(Cc2ccc(C3CCCCC3)cn2)c2ccccc2)cc(F)c1F]

Step 1. To 2,3-difluorobenzoic acid (3.5 g) was added concentrated H2SO4 (17 ml) and N-bromosuccinimide (4.33 g). The reaction mixture was heated with stirring at 60° C. for three hours under argon. Reaction was then allowed to cool to room temperature and poured onto ice water. This mixture was allowed to stir at room temperature for five minutes, and then filtered. The solid was then washed with room temperature water. The solid was then dissolved in ethyl acetate and extracted with 3M sodium hydroxide. The ethyl acetate layer was then discarded, and the aqueous layer was then acidified with 3M HCl. The aqueous layer was extracted with ethyl acetate. The combined extracts were washed with water, brine, dried over Na2SO4 and concentrated to obtain 5-bromo-2,3-difluorobenzoic acid as an impure mixture arising from a lack of regio-specificity in the bromination (4.39 g, 84% yield).

Step 2. To a solution of 5-bromo-2,3-difluorobenzoic acid (4.29 g) in dichloromethane (30 ml) was added oxalyl chloride (2.15 ml) followed by dry DMF (4 drops) under argon. The mixture was allowed to stir at room temperature for 2.5 hours and then concentrated. Dry acetonitrile (20 ml) was added, and the solution was poured onto a solution of dimethylamine (excess) in acetonitrile (10 ml). The mixture was allowed to reach room temperature and then stirred for 15 minutes. Water was added to the mixture and then it was extracted with ethyl acetate. The extracts were then washed with water, brine, dried over Na2SO4 and concentrated to obtain 5-bromo-2,3-difluoro-N,N-dimethylbenzamide (3.52 g, 75% yield).

Step 3. To 5-bromo-2,3-difluoro-N,N-dimethylbenzamide (725 mg) was added Pd2(dba)3 (128 mg) and Xantphos (161 mg). The reaction vessel was then flushed with argon. To the solids was added dioxane (10 ml), followed by i-Pr2NEt (0.975 ml), and benzyl mercaptan (0.425 ml). The reaction mixture was allowed to stir at 101° C. for 19 hours. After cooling to room temperature, water was added, and the mixture was then extracted with ethyl acetate. The combined organic extracts were washed with water, brine, dried and concentrated. Purification by column chromatography (1:4 ethyl acetate/hexanes) to resolve isomeric impurities gave 5-(benzylthio)-2,3-difluoro-N,N-dimethylbenzamide (701 mg, 83% Yield).

Step 4. To a solution of 5-(benzylthio)-2,3-difluoro-N,N-dimethylbenzamide (710 mg) in HPLC acetonitrile (30 ml) was added acetic acid (0.529 ml) and HPLC water (0.29 ml). The mixture was cooled to 0° C. and 1,3,5-trichloro-1,3,5-triazinane-2,4,6-trione was added (1.073 g). The ice bath was removed and the reaction was stirred for one hour. Added water to the reaction and extracted with ethyl acetate. The organic extracts were washed with pH 7 buffer, water, brine, dried, and concentrated. Purification by column chromatography (1:4 ethyl acetate/hexanes) gave 3-(dimethylcarbamoyl)-4,5-difluorobenzenesulfonyl chloride (291 mg, 45% yield). 1H NMR (300 MHz, CDCl3) δ 7.98-7.91 (m, 2H), 3.19 (s, 3H), 3.00 (s, 3H). 19F NMR (282 MHz, CDCl3) δ−125.90-−126.57 (m), −128.95-−129.55 (m).

Step 5. To 2,2,2-trifluoro-N-phenylacetamide (1.323 mg) under argon was added NaI (312 mg) and Cs2CO3 (10.237 g). The solids were then dissolved in acetonitrile (50 ml). A 1M solution of (chloromethyl)-5-cyclohexylpyridine (14 ml) in toluene as a 1M solution was added to the mixture which was then heated to 65° C. and allowed to stir for sixteen hours. The reaction was allowed to cool to room temperature. Saturated ammonium chloride solution was added, and the reaction mixture was then extracted with ethyl acetate. The combined organic extracts were then washed with water, brine, and died over Na2SO4. Purification by column chromatography (2:8 ethyl acetate/hexanes) gave N-((5-cyclohexylpyridin-2-yl)methyl)-2,2,2-trifluoro-N-phenylacetamide as a crude mixture with some starting material (2,2,2-trifluoro-N-phenylacetamide).

Step 6. To crude N-((5-cyclohexylpyridin-2-yl)methyl)-2,2,2-trifluoro-N-phenylacetamide (2.822 mg) under argon was added K2CO3 (2.867 mg) followed by THF (20 ml) and methanol (20 ml). The resulting mixture was allowed to stir at room temperature for four hours. Saturated ammonium chloride solution was added. The reaction mixture was then extracted with ethyl acetate. The combined organic extracts were then washed with water, brine, and dried over Na2SO4. Purification by column chromatography (2:8 ethyl acetate/hexanes) gave N-((5-cyclohexylpyridin-2-yl)methyl)aniline (1.131 g, 47% yield over two steps).

Step 7. To a stirred solution of N-((5-cyclohexylpyridin-2-yl)methyl)aniline (455 mg) in THF (8 ml) at 0° C. under argon was added a solution of 1.03 M MeMgBr (2.7 ml) in 1:3 toluene:tetrahydrofuran. The reaction mixture was allowed to stir for fifteen minutes before tert-butyl (R)-2-(chlorocarbonyl)azetidine-1-carboxylate (709 mg) in THF (8 ml) was added. The ice bath was then removed and the reaction was allowed to reach room temperature. After two and a half hours saturated ammonium chloride solution was added. The reaction mixture was then extracted with ethyl acetate. The combined organic extracts were then washed with water, brine, and dried over Na2SO4. Purification by column chromatography (4:6 ethyl acetate/hexanes) gave tert-butyl (R)-2-(((5-cyclohexylpyridin-2-yl)methyl)(phenyl)carbamoyl)azetidine-1-carboxylate (138 mg, 18% yield).

Step 8. To a solution of tert-butyl (R)-2-(((5-cyclohexylpyridin-2-yl)methyl)(phenyl)carbamoyl)azetidine-1-carboxylate (133 mg) in DCM (10 ml) under argon was added TFA (1 ml). The reaction mixture was stirred at room temperature for one hour. The reaction was then concentrated and the resulting solid ((R)—N-((5-cyclohexylpyridin-2-yl)methyl)-N-phenylazetidine-2-carboxamide TFA salt) was used directly in the next reaction.

Step 9. The solid ((R)—N-((5-cyclohexylpyridin-2-yl)methyl)-N-phenylazetidine-2-carboxamide TFA salt) was dissolved in DCM (10 ml), and DIPEA (0.31 ml) was added to the solution at 0° C. The reaction mixture was allowed to stir for fifteen minutes 3-(dimethylcarbamoyl)-4,5-difluorobenzenesulfonyl chloride (169 mg) in DCM (10 ml) was added. The ice bath was removed and the reaction was allowed to warm to room temperature. After two and a half hours saturated ammonium chloride solution was added. The reaction mixture was extracted with dichloromethane. The combined organic extracts were washed with water, brine, and dried over Na2SO4. Purification by column chromatography (27:63 acetone/hexanes) gave (R)—N-((5-cyclohexylpyridin-2-yl)methyl)-1-((5-(dimethylcarbamoyl)-2,4-difluorophenyl)sulfonyl)-N-phenylazetidine-2-carboxamide (3 mg of pure fraction, 2% yield over two steps at this purity). 1H NMR (300 MHz, CDCl3) δ 8.35 (d, J=2.2 Hz, 1H), 7.90-7.81 (m, 1H), 7.72 (dt, J=4.9, 1.9 Hz, 1H), 7.57 (dd, J=8.1, 2.2 Hz, 1H), 7.44-7.33 (m, 3H), 7.16 (dd, J=7.0, 2.6 Hz, 2H), 5.13-4.94 (m, 2H), 4.85 (t, J=8.3 Hz, 1H), 3.85 (q, J=8.1 Hz, 1H), 3.70 (td, J=8.0, 7.1, 3.9 Hz, 1H), 3.15 (s, 3H), 2.95 (d, J=1.3 Hz, 3H), 2.53 (s, 1H), 2.47-2.33 (m, 1H), 1.81 (m, 6H), 1.50-1.31 (m, 5H). 19F NMR (282 MHz, CDCl3) δ−131.69 (d, J=22.1 Hz), −132.05-−132.20 (m). LCMS (100% purity) (ESI) m/z 597.3 [M+H]+.

### Example 87

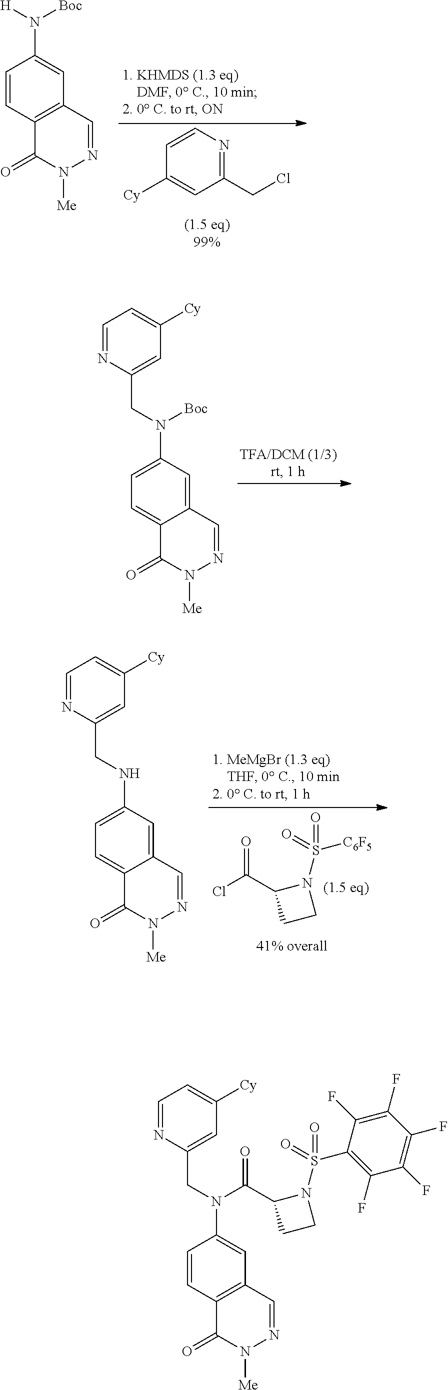

### (R)—N-((4-cyclohexylpyridin-2-yl)methyl)-N-(2-methyl-1-oxo-1,2-dihydrophthalazin-6-yl)-1-((perfluorophenyl)sulfonyl)azetidine-2-carboxamide

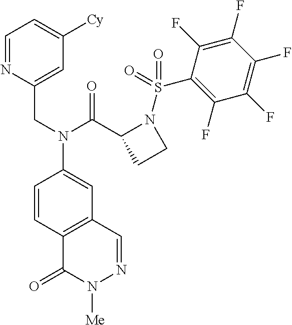

Step 1: To a solution of tert-butyl (2-methyl-1-oxo-1,2-dihydrophthalazin-6-yl)carbamate (146 mg, 0.53 mmol, 1.0 equiv) in 5 mL DMF was added KHMDS (0.7 mL, 0.69 mmol, 1.3 equiv, 1.0 M in THF) at 0° C. dropwise under Argon. 10 minutes late, the solution of 2-(chloromethyl)-4-cyclohexylpyridine (0.8 mmol, 1.5 equiv, 0.5 M in toluene) was added to the reaction mixture. The reaction was allowed to warm up to room temperature and stirred at room temperature overnight. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 1/1) to provide tert-butyl ((4-cyclohexylpyridin-2-yl)methyl) (2-methyl-1-oxo-1,2-dihydrophthalazin-6-yl)carbamate as light yellow oil (237 mg, 99%). 1H NMR (300 MHz, CDCl3) δ 8.44 (d, J=5.3 Hz, 1H), 8.33 (d, J=8.7 Hz, 1H), 8.06 (s, 1H), 7.83-7.66 (m, 2H), 7.16 (s, 1H), 7.08 (d, J=5.0 Hz, 1H), 5.07 (s, 2H), 3.83 (s, 3H), 2.58-2.44 (m, 1H), 1.92-1.73 (m, 5H), 1.60-1.18 (m, 14H).

Step 2: To a solution of tert-butyl ((4-cyclohexylpyridin-2-yl)methyl) (2-methyl-1-oxo-1,2-dihydrophthalazin-6-yl)carbamate (235 mg) in 6 mL DCM was added 2.3 mL TFA. The reaction was stirred at room temperature for 1 h. Then the reaction was concentrated under reduced pressure, diluted with dry DCE and concentrated again. The residue was dried under high vacuum for 30 minutes and used directly for the next step.

Step 3. To a solution of the above residue in 5 mL THF was added MeMgBr (0.5 mL, 0.68 mmol, 1.3 equiv, 1.4 M in THF/toluene) dropwise at 0° C. under Argon. After 10 minutes, (R)-1-((perfluorophenyl)sulfonyl)azetidine-2-carbonyl chloride (276 mg, 0.79 mmol, 1.5 equiv) was added at 0° C. under Argon. The reaction was allowed to warm up to room temperature and stirred at room temperature for 1 h. Then the reaction was quenched with water and extracted with EtOAc (3×). The combined organic extracts were washed with saturated brine, dried over anhydrous sodium sulfate, and concentrated under reduced pressure. The resulting residue was purified by flash chromatography (eluent: hexane/acetone 3/1) to provide (R)—N-((4-cyclohexylpyridin-2-yl)methyl)-N-(2-methyl-1-oxo-1,2-dihydrophthalazin-6-yl)-1-((perfluorophenyl)sulfonyl)azetidine-2-carboxamide as light yellow foam (143 mg, 41% over 3 steps). ¹H NMR (300 MHz, CDCl3) δ 8.41 (d, J=8.4 Hz, 1H), 8.34 (d, J=4.5 Hz, 1H), 8.08 (s, 1H), 7.61 (s, 1H), 7.55 (dd, J=8.5, 1.1 Hz, 1H), 7.12 (s, 1H), 7.03 (d, J=4.8 Hz, 1H), 4.97 (s, 3H), 4.17-4.00 (m, 2H), 3.85 (s, 3H), 2.55-2.42 (m, 1H), 2.41-2.26 (m, 1H), 1.88-1.68 (m, 7H), 1.49-1.29 (m, 4H). LRMS (ESI) m/z 662.2 [M+H]+; Purity 96%.

### Example 88

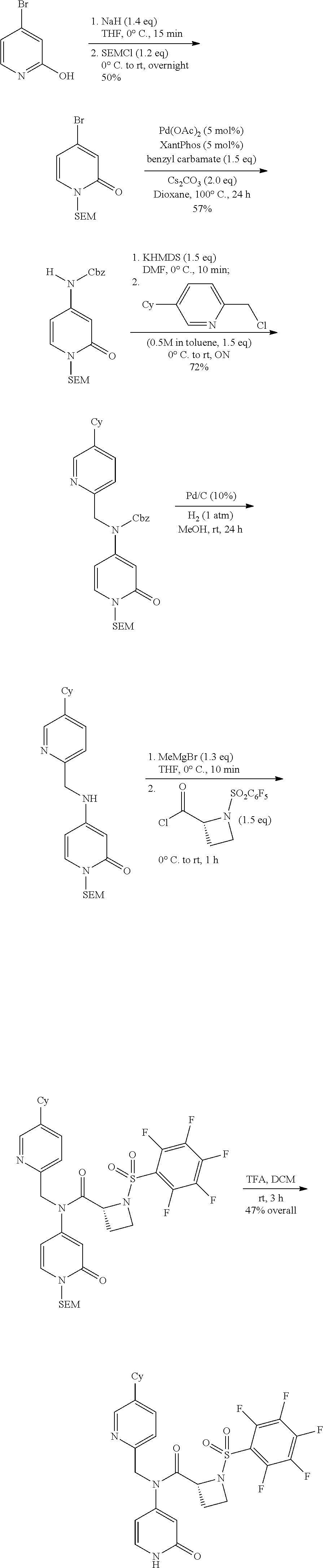

### (R)—N-((5-cyclohexylpyridin-2-yl)methyl)-N-(2-oxo-1,2-dihydropyridin-4-yl)-1-((perfluorophenyl)sulfonyl)azetidine-2-carboxamide

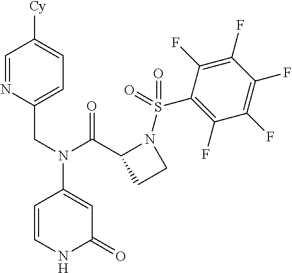

Step 1: To a solution of 4-bromopyridin-2-ol (521 mg, 2.99 mmol, 1.0 equiv) in 15 mL THF was added NaH (162 mg, 4.19 mmol, 1.4 equiv, 60% in mineral oil) slowly at 0° C. under Argon. After 15 minutes, SEMCl (0.64 mL, 3.59 mmol, 1.2 equiv) was added at 0° C. Then the reaction was allowed to warm up to room temperature and stirred for overnight. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc 7/3) to obtain 4-bromo-1-((2-(trimethylsilyl)ethoxy)methyl)pyridin-2(1H)-one as white solid (458 mg, 50%). ¹H NMR (300 MHz, CDCl3) δ 7.31 (d, J=7.3 Hz, 1H), 6.84 (d, J=2.0 Hz, 1H), 6.39 (dd, J=7.3, 2.1 Hz, 1H), 5.32 (s, 2H), 3.63 (t, J=8.3 Hz, 2H), 0.96 (t, J=8.4 Hz, 2H), 0.02 (s, 9H).

Step 2: 4-bromo-1-((2-(trimethylsilyl)ethoxy)methyl)pyridin-2(1H)-one (450 mg, 1.48 mmol, 1.0 equiv), benzyl carbamate (335 mg, 2.22 mmol, 1.5 equiv), and Cs2CO3 (964 mg, 2.96 mmol, 2.0 equiv) were dissolved in 15 mL 1,4-dioxane. After 10 minutes, Pd(OAc)2 (17 mg, 0.074 mmol, 5 mol %) and XantPhos (43 mg, 0.074 mmol, 5 mol %) were added to the reaction under Argon. Then the reaction was heated at 100° C. for 24 h. The reaction was quenched with water, extracted with ethyl acetate (3×). The combined organic extracts were washed with saturated brine, dried over anhydrous sodium sulfate, and concentrated under reduced pressure. The resulting residue was purified by flash chromatography (eluent: hexane/EtOAc/MeOH 1.5/1/3%) to provide benzyl (2-oxo-1-((2-(trimethylsilyl)ethoxy)methyl)-1,2-dihydropyridin-4-yl)carbamate as white solid (315 mg, 57%). ¹H NMR (300 MHz, CDCl3) δ 7.43-7.31 (m, 5H), 7.07 (s, 1H), 6.67 (dd, J=7.5, 2.1 Hz, 1H), 6.42 (d, J=2.3 Hz, 1H), 5.29 (s, 2H), 5.19 (s, 2H), 3.60 (t, J=8.3 Hz, 2H), 0.92 (t, J=8.3 Hz, 2H), −0.02 (s, 9H).

Step 3: To a solution of benzyl (2-oxo-1-((2-(trimethylsilyl)ethoxy)methyl)-1,2-dihydropyridin-4-yl)carbamate (150 mg, 0.40 mmol, 1.0 equiv) in 4 mL DMF was added KHMDS (0.5 mL, 0.52 mmol, 1.3 equiv, 1.0 M in THF) at 0° C. dropwise under Argon. 10 minutes late, 2-(chloromethyl)-5-cyclohexylpyridine (1.2 mL, 0.6 mmol, 1.5 equiv, 0.5 M in toluene) was added to the reaction mixture. The reaction was allowed to warm up to room temperature and stirred at room temperature for 24 h. Then the reaction was quenched with saturated NH4Cl aqueous solution and extracted with ethyl acetate (3×). The combined organic extracts were washed with saturated brine, dried over anhydrous sodium sulfate, and concentrated under reduced pressure. The resulting residue was purified by flash chromatography (eluent: hexane/acetone 2/1) to provide benzyl ((5-cyclohexylpyridin-2-yl)methyl) (2-oxo-1-((2-(trimethylsilyl)ethoxy)methyl)-1,2-dihydropyridin-4-yl)carbamate as light yellow oil (158 mg, 72%). 1H NMR (300 MHz, CDCl3) δ 8.38 (d, J=2.0 Hz, 1H), 7.42 (dd, J=8.0, 2.0 Hz, 1H), 7.31-7.26 (m, 4H), 7.24-7.16 (m, 2H), 7.03 (d, J=8.1 Hz, 1H), 6.66 (dd, J=7.7, 2.5 Hz, 1H), 6.29 (d, J=2.5 Hz, 1H), 5.27 (s, 2H), 5.20 (s, 2H), 4.99 (s, 2H), 3.65-3.54 (m, 2H), 2.56-2.45 (m, 1H), 1.91-1.78 (m, 6H), 1.47-1.35 (m, 4H), 0.98-0.87 (m, 2H), −0.05 (s, 9H).

Step 4: Step 3: benzyl ((5-cyclohexylpyridin-2-yl)methyl)(2-oxo-1-((2-(trimethylsilyl)ethoxy)methyl)-1,2-dihydropyridin-4-yl)carbamate (158 mg) and Pd/C (15 mg) were dissolved in 5 mL MeOH under hydrogen gas (1 atm). After 24 h, the catalyst was filtered off through a celite pad and washed with ethyl acetate. The combined solvent was concentrated under reduced pressure to obtain crude product 4-(((5-cyclohexylpyridin-2-yl)methyl)amino)-1-((2-(trimethylsilyl)ethoxy)methyl)pyridin-2(1H)-one as off-white solid (120 mg), which was used directly for next step without any further purification.

Step 5. To a solution of 4-(((5-cyclohexylpyridin-2-yl)methyl)amino)-1-((2-(trimethylsilyl)ethoxy)methyl)pyridin-2(1H)-one (42 mg, 0.10 mmol, 1.0 equiv) in 1 mL THF was added MeMgBr (0.1 mL, 0.13 mmol, 1.3 equiv, 1.4 M in THF/toluene) dropwise at 0° C. under Argon. After 10 minutes, (R)-1-((perfluorophenyl)sulfonyl)azetidine-2-carbonyl chloride (53 mg, 0.15 mmol, 1.5 equiv) was added at 0° C. under Argon. The reaction was allowed to warm up to room temperature and stirred at room temperature for 1 h. Then the reaction was quenched with water and extracted with EtOAc (3×). The combined organic extracts were washed with saturated brine, dried over anhydrous sodium sulfate, and concentrated under reduced pressure to provide crude product (R)—N-((5-cyclohexylpyridin-2-yl)methyl)-N-(2-oxo-1-((2-(trimethylsilyl)ethoxy)methyl)-1,2-dihydropyridin-4-yl)-1-((perfluorophenyesulfonyl)azetidine-2-carboxamide, which was used directly for next step.

Step 6. To the solution of the above residue in 3 mL DCM was added 0.8 mL TFA. The reaction was stirred at room temperature for 3 h. Then the reaction was concentrated under reduced pressure, then quenched with saturated Na2CO3 aqueous solution. The reaction was extracted with DCM (3×). The combined organic extracts were washed with saturated brine, dried over anhydrous sodium sulfate, and concentrated under reduced pressure. The resulting residue was purified by flash chromatography (eluent: hexane/acetone 1/1) to provide (R)—N-((5-cyclohexylpyridin-2-yl)methyl)-N-(2-oxo-1,2-dihydropyridin-4-yl)-1-((perfluorophenyl)sulfonyl)azetidine-2-carboxamide as white foam (28 mg, 47%). 1H NMR (300 MHz, CDCl3) δ 12.80 (s, 1H), 8.34 (s, 1H), 7.51 (d, J=7.5 Hz, 1H), 7.36 (d, J=6.7 Hz, 1H), 7.16 (d, J=7.9 Hz, 1H), 6.43-6.23 (m, 2H), 5.32-5.21 (m, 1H), 4.89 (s, 2H), 4.28-4.03 (m, 2H), 2.60-2.46 (m, 1H), 2.45-2.28 (m, 2H), 1.96-1.77 (m, 6H), 1.51-1.32 (m, 4H). LRMS (ESI) m/z 597.3 [M+H]+; Purity 96%.

### Example 89: In Vitro and In Vivo Analysis of the Effects on Stat3-Harboring Cells Using Compounds of this Disclosure

The methods and reagents described herein can be further described in Siddiquee, K., et al. Proc Natl Acad Sci USA. 2007 May 1; 104(18): 7391-7396, herein incorporated by reference as to the materials and methods described therein.

Cell lines and reagents. Normal mouse fibroblast (NIH3T3), and the human breast cancer MDA-MB-231, MDA-MB-468, MCF-7, and MCF-10A cell lines have been reported previously (Zhang X, et al., (2010) Biochem Pharmacol 79:1398-409; Zhang X, et al., (2012) Proc Natl Acad Sci USA 109:9623-8; Garcia R, et al., (1997) Cell Growth Diff. 8:1267-1276; Garcia R, et al., (2001) Oncogene 20:2499-2513, all of which are hereby incorporated by reference). Cells were grown in Dulbecco's modified Eagle's medium (DMEM) containing 10% heat-inactivated fetal bovine serum (for human cells) or bovine calf serum (for mouse cells). Human metastatic melanoma were maintained in RPMI with 10% FBS. Human metastatic melanoma C8161, 1205Lu, UACC903 cell lines, C81-61, an early stage melanoma from the same patient as the late stage C8161 cells, have been reported (Cui M, et al., (2013) Multifunctional albumin nanoparticles as combination drug carriers for intra-tumoral chemotherapy. Adv Healthc Mater 2:1236-41; Lee H J, et al. (2011) Glutamatergic pathway targeting in melanoma: single-agent and combinatorial therapies. Clin Cancer Res. 17:7080-92) and were maintained in RPMI with 10% FBS. The immortalized normal human melanocytes, AR7119 line was maintained in Medium 254 supplemented with human growth factors (GIBCO). Antibodies against STATS, pSTAT3, Src, pSrc Jak, pJak, ErkMAPK, and pErkMAPK were purchased from Cell Signaling Technology, Inc. (Danvers, Mass.). Tubulin was purchased from (Santa Cruz Biotechnology, Inc., Dallas, Tex.).

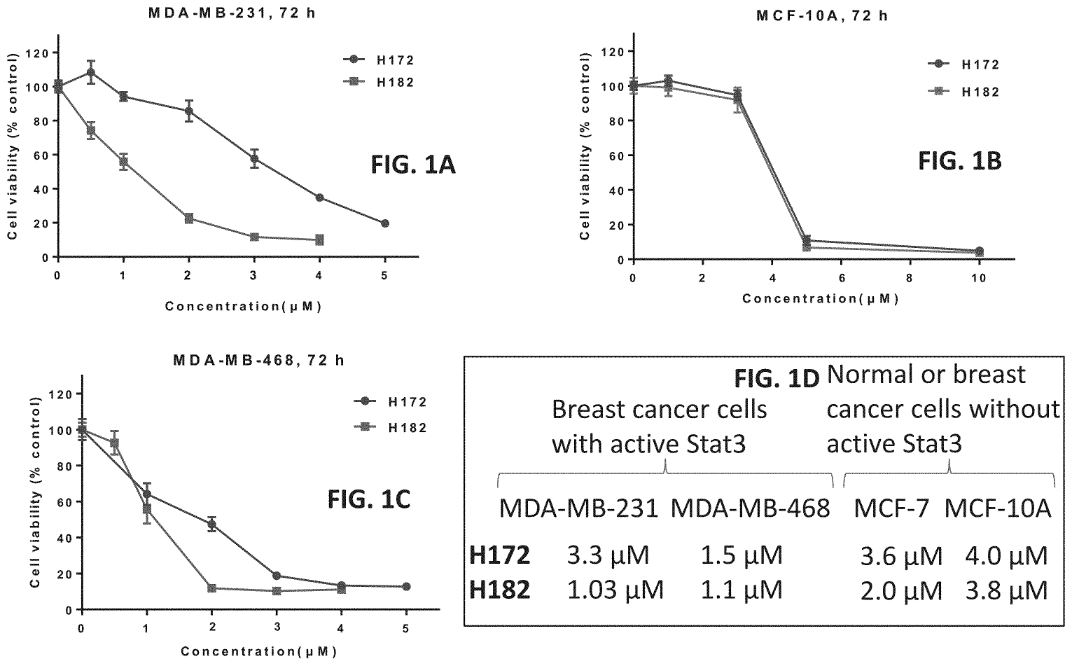

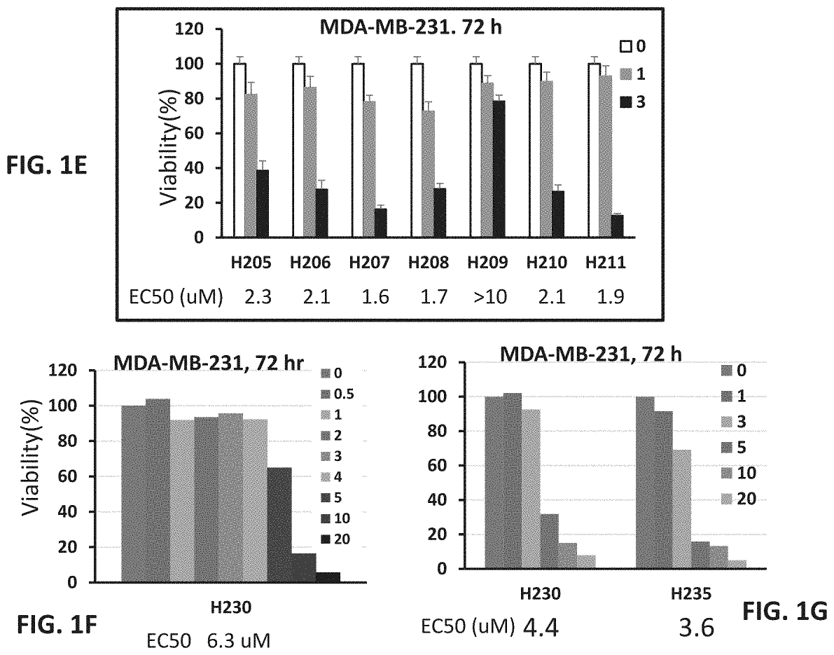

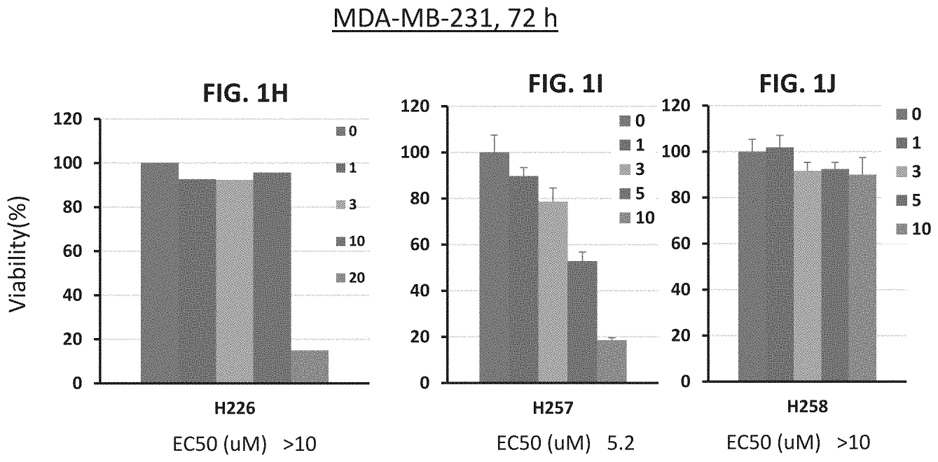

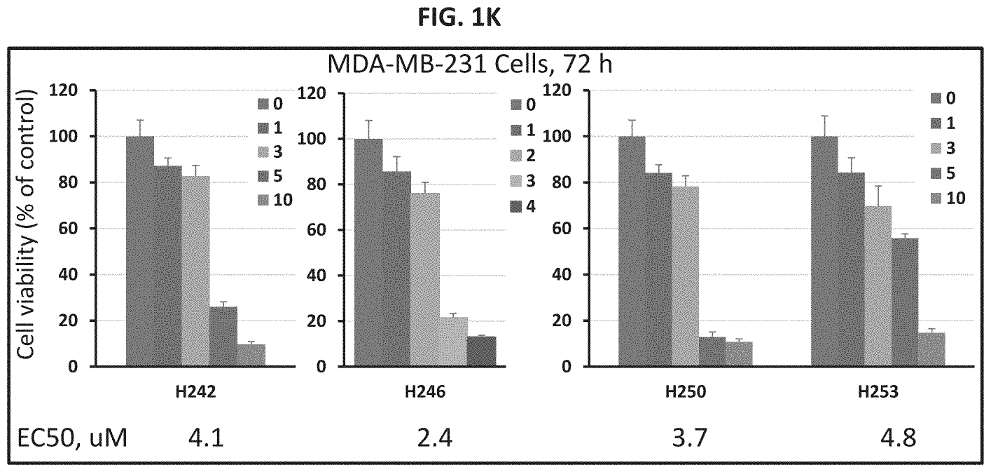

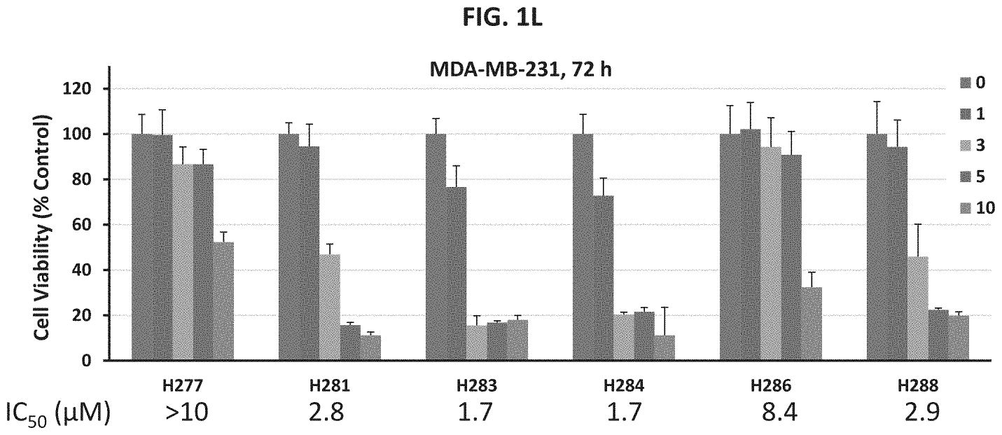

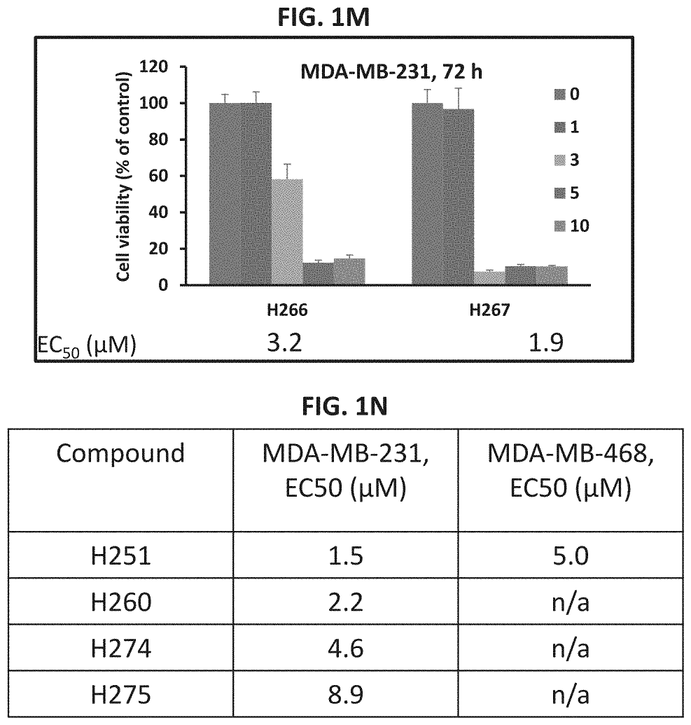

As shown in FIGS. 1A-1N, the effects of compounds on cell viability in vitro relative to control (untreated cells) indicate that the compounds were more potent to cells harboring active Stat3 than vehicle alone. The cells lines used were those of melanoma cells harboring aberrantly-active Stat3 (MDA-MB-231, MDA-MB-468) and counterpart that does not (MCF-7 and MCF-10A). Cell numbers were counted by trypan blue exclusion/phase-contrast microscopy every 34 h, and cell growth curve was plotted against concentration from which IC50 values were derived. FIG. 1A shows that MDA-MB-231 cells in 6-well plates were treated once with 0.05% DMSO, or 0.5, 1, 2, 3, 4, 5 and 10 μM of the aryl sulfonamido azetidine compounds H172 or H182 for 0-72 h. FIG. 1B shows that MCF-10A cells in 6-well plates were treated once with 0.05% DMSO, or 0.5, 1, 2, 3, 4, 5 and 10 μM of compound of H172 or H182 for 0-72 h. FIG. 1C shows that MDA-MB-468 cells in 6-well plates were treated once with 0.05% DMSO, or 0.5, 1, 2, 3, 4, 5 and 10 μM of compound of H172 or H182 for 0-72 h. FIG. 1D shows the summary of the relative IC50 values (in micromolar) against cell lines harboring Stat3 activity compared to the cell lines without active Stat3. FIGS. 1E-1N show the cell viability analysis to MDA-MB-231 or MDA-MB-468 breast cancer cells for representative aryl sulfonamido compounds described herein, demonstrating that the compounds are potent in treating breast cancer cells harboring Stat3.

Nuclear extract preparation, gel shift assays, and densitometric analysis. Nuclear extract preparations and DNA-binding activity/electrophoretic mobility shift assay (EMSA) were carried out as previously described (Zhang X, et al., (2010) Biochem Pharmacol 79:1398-409; Zhang X, et al., (2012) Proc Natl Acad Sci USA 109:9623-8). The ³²P-labeled oligonucleotide probes used were hSIE (high affinity sis-inducible element from the c-fos gene, m67 variant, 5′-AGCTTCATTTCCCGTAAATCCCTA) (SEQ ID NO: 1) that binds Stat1 and Stat3 and MGFe (mammary gland factor element from the bovine β-casein gene promoter, 5′-AGATTTCTAGGAATTCAA) (SEQ ID NO: 2) for Stat1 and Stat5 binding. Except where indicated, nuclear extracts were pre-incubated with compound for 30 min at room temperature prior to incubation with the radiolabeled probe for 30 min at 30° C. before subjecting to EMSA analysis. Where appropriate, bands corresponding to Stat3:DNA complexes were scanned and quantified for each concentration of compound using ImageJ and plotted as percent of control (DMSO) against concentration of compound, from which the IC50 values were derived.

Immunoblotting analysis. Whole cell lysate preparation and immunoblotting analysis were performed as previously reported (Zhang X, et al., (2010) Biochem Pharmacol 79:1398-409; Zhang X, et al., (2012) Proc Natl Acad Sci USA 109:9623-8). All antibodies tested were purchased from Cell Signaling Technology.

Cell proliferation and viability assay. Studies were conducted as previously reported (Zhang X, et al., (2010) Biochem Pharmacol 79:1398-409; Zhang X, et al., (2012) Proc Natl Acad Sci USA 109:9623-8; Siddiquee K, et al., (2007) Proc Natl Acad Sci USA 104:7391-6; Siddiquee K A, et al., (2007) ACS Chem Biol 2:787-98). Cells in 6-well or 96-well plates were treated with or without compounds for the indicated concentrations and time, and subjected to MTT assay or CyQuant cell proliferation assay (Invitrogen/ThermoFisher Scientific), or harvested and the viable cells were counted by trypan blue exclusion with phase-contrast microscopy.

Clonogenic survival assays. Colony survival assay was performed as previously reported (Zhang X, et al., (2010) Biochem Pharmacol 79:1398-409; Zhang X, et al., (2012) Proc Natl Acad Sci USA 109:9623-8). Briefly, cells were seeded as single-cell cultures in 6-cm dishes (250 cells per dish), treated once the next day with compounds at the indicated concentrations and allowed to culture until large colonies were visible. Colonies were stained with crystal violet for 4 h, counted and photographed.

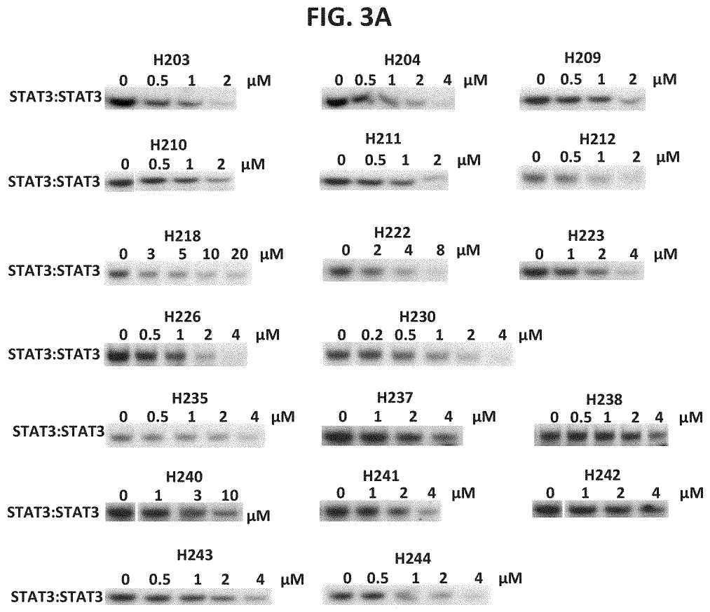

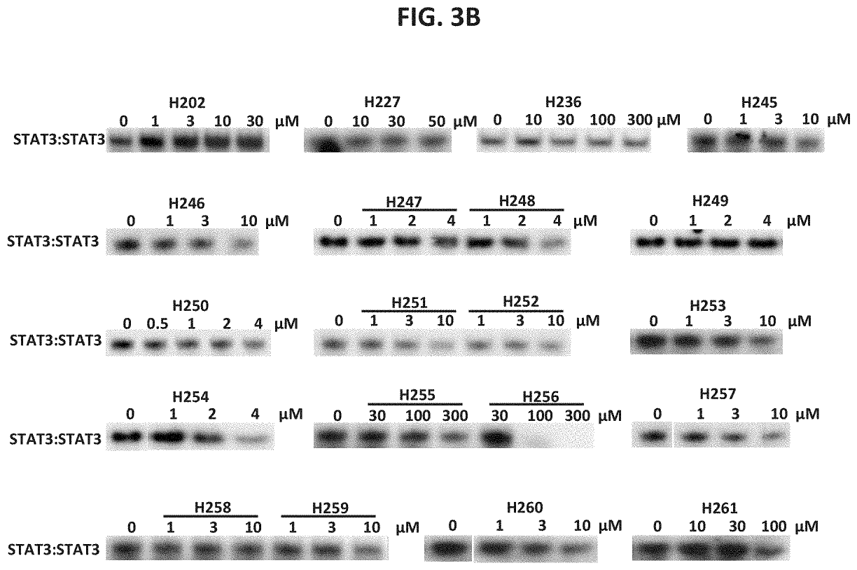

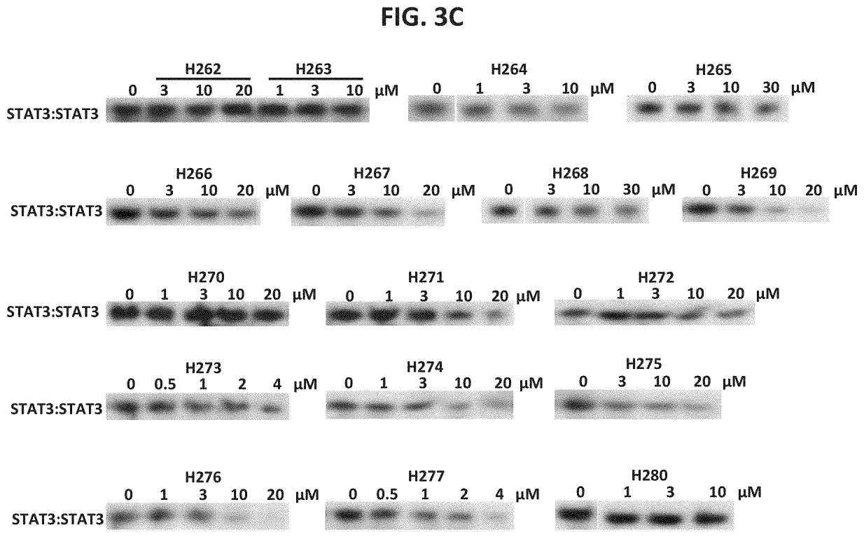

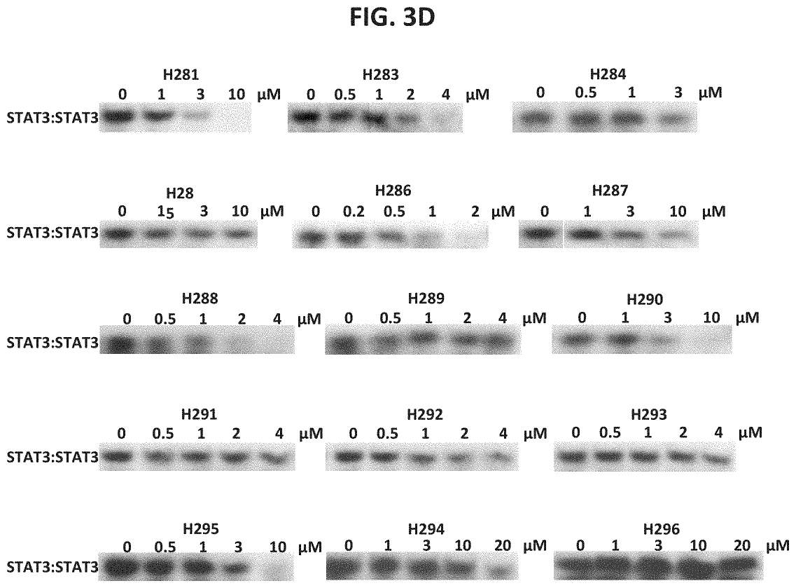

Formulae I-V were designed around a core scaffold to develop potent Stat3 inhibitor compounds with appropriate physicochemical properties. The inhibitory activities of the representative analogs against in vitro Stat3 DNA-binding activity, as measured by electrophoretic mobility shift assay (EMSA) for representative compounds from the Examples above are shown in FIGS. 3A-3D and summarized in Table 1.

| | also | | EMSA |
|---|---|---|---|
| Ex- | referred to | | IC₅₀ |
| ample | herein as | Chemical Structure | (uM) |
| 1 | H203 | 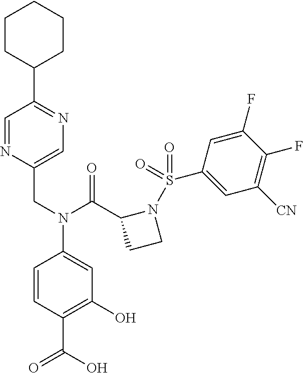 [SMILES: N#Cc1cc(S(=O)(=O)N2CC[C@@H]2C(=O)N(Cc2cnc(C3CCCCC3)cn2)c2ccc(C(=O)O)c(O)c2)cc(F)c1F] | 0.283 ± 0.031 |
| 2 | H216 | 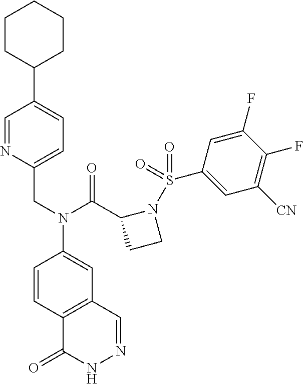 [SMILES: N#Cc1cc(S(=O)(=O)N2CC[C@@H]2C(=O)N(Cc2ccc(C3CCCCC3)cn2)c2ccc3c(=O)[nH]ncc3c2)cc(F)c1F] | 0.590 ± 0.079 |
| 3 | H217 | 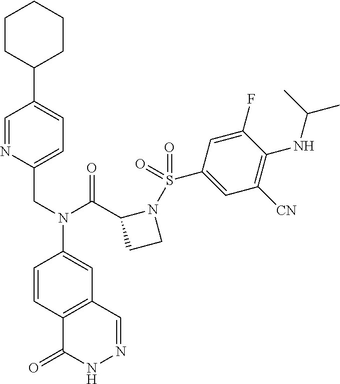 [SMILES: CC(C)Nc1c(F)cc(S(=O)(=O)N2CC[C@@H]2C(=O)N(Cc2ccc(C3CCCCC3)cn2)c2ccc3c(=O)[nH]ncc3c2)cc1C#N] | 10.9 ± 2.2 |
| 4 | H223 | 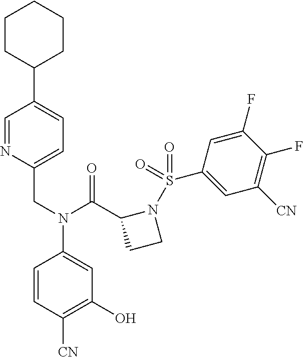 [SMILES: N#Cc1ccc(N(Cc2ccc(C3CCCCC3)cn2)C(=O)[C@H]2CCN2S(=O)(=O)c2cc(F)c(F)c(C#N)c2)cc1O] | 1.29 ± 0.20 |
| 5 | H238 | 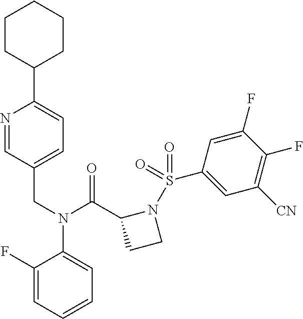 [SMILES: N#Cc1cc(S(=O)(=O)N2CC[C@@H]2C(=O)N(Cc2ccc(C3CCCCC3)nc2)c2ccccc2F)cc(F)c1F] | 3.2 ± 0.2 |
| 6 | H242 | 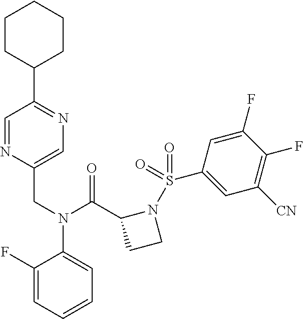 [SMILES: N#Cc1cc(S(=O)(=O)N2CC[C@@H]2C(=O)N(Cc2cnc(C3CCCCC3)cn2)c2ccccc2F)cc(F)c1F] | >4 |
| 7 | H209 | 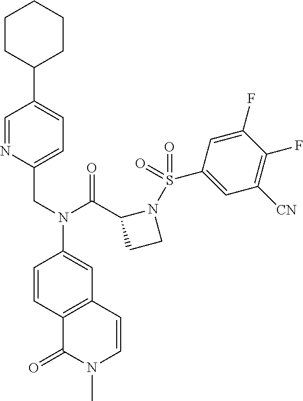 [SMILES: Cn1ccc2cc(N(Cc3ccc(C4CCCCC4)cn3)C(=O)[C@H]3CCN3S(=O)(=O)c3cc(F)c(F)c(C#N)c3)ccc2c1=O] | 0.884 ± 0.081 |
| 8 | H210 | 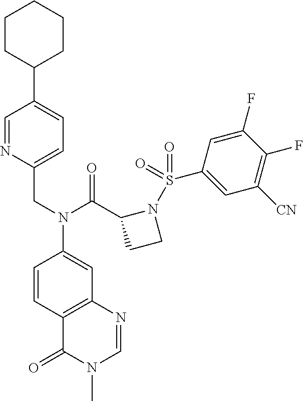 [SMILES: Cn1cnc2cc(N(Cc3ccc(C4CCCCC4)cn3)C(=O)[C@H]3CCN3S(=O)(=O)c3cc(F)c(F)c(C#N)c3)ccc2c1=O] | 0.971 ± 0.063 |
| 9 | H211 | 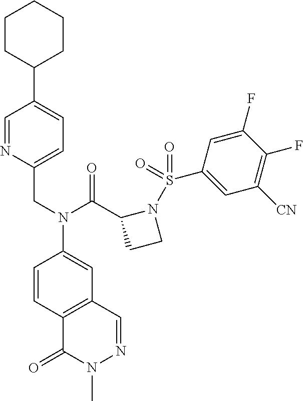 [SMILES: Cn1ncc2cc(N(Cc3ccc(C4CCCCC4)cn3)C(=O)[C@H]3CCN3S(=O)(=O)c3cc(F)c(F)c(C#N)c3)ccc2c1=O] | 0.799 ± 0.077 |
| 10 | H226 | 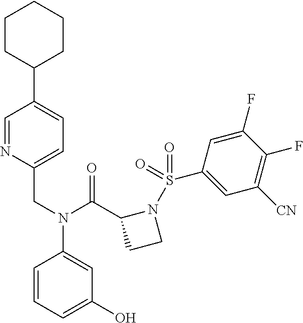 [SMILES: N#Cc1cc(S(=O)(=O)N2CC[C@@H]2C(=O)N(Cc2ccc(C3CCCCC3)cn2)c2cccc(O)c2)cc(F)c1F] | 0.780 ± 0.132 |
| 11 | H243 | 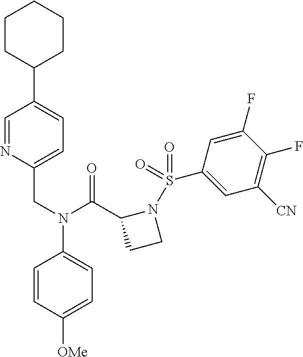 [SMILES: COc1ccc(N(Cc2ccc(C3CCCCC3)cn2)C(=O)[C@H]2CCN2S(=O)(=O)c2cc(F)c(F)c(C#N)c2)cc1] | 1.21 ± 0.31 |
| 12 | H240 | 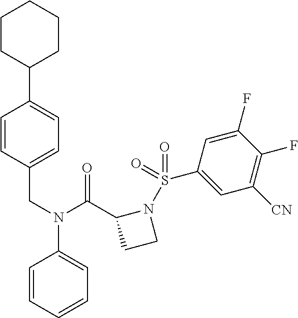 [SMILES: N#Cc1cc(S(=O)(=O)N2CC[C@@H]2C(=O)N(Cc2ccc(C3CCCCC3)cc2)c2ccccc2)cc(F)c1F] | 3.3 ± 0.2 |
| 13 | H241 | 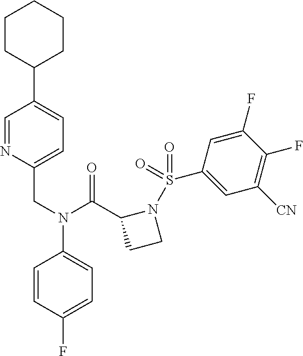 [SMILES: N#Cc1cc(S(=O)(=O)N2CC[C@@H]2C(=O)N(Cc2ccc(C3CCCCC3)cn2)c2ccc(F)cc2)cc(F)c1F] | 2.2 ± 0.6 |
| 14 | H204 | 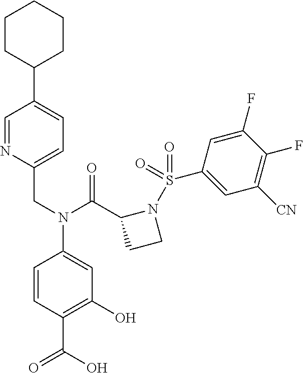 [SMILES: N#Cc1cc(S(=O)(=O)N2CC[C@@H]2C(=O)N(Cc2ccc(C3CCCCC3)cn2)c2ccc(C(=O)O)c(O)c2)cc(F)c1F] | 0.520 ± 0.016 |
| 15 | H212 | 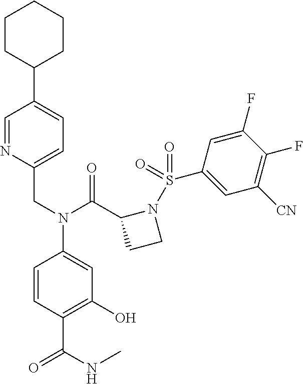 [SMILES: CNC(=O)c1ccc(N(Cc2ccc(C3CCCCC3)cn2)C(=O)[C@H]2CCN2S(=O)(=O)c2cc(F)c(F)c(C#N)c2)cc1O] | 0.711 ± 0.074 |
| 16 | H222 | 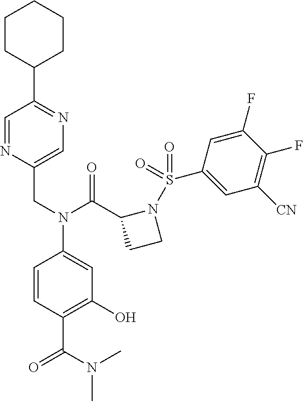 [SMILES: CN(C)C(=O)c1ccc(N(Cc2cnc(C3CCCCC3)cn2)C(=O)[C@H]2CCN2S(=O)(=O)c2cc(F)c(F)c(C#N)c2)cc1O] | 2.25 ± 0.04 |
| 17 | H230 | 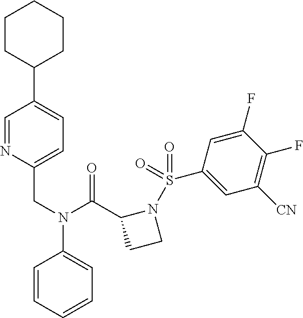 [SMILES: N#Cc1cc(S(=O)(=O)N2CC[C@@H]2C(=O)N(Cc2ccc(C3CCCCC3)cn2)c2ccccc2)cc(F)c1F] | 0.632 ± 0.052 |
| 18 | H235 | 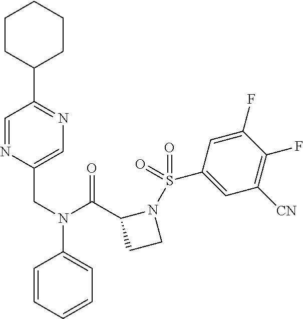 [SMILES: N#Cc1cc(S(=O)(=O)N2CC[C@@H]2C(=O)N(Cc2cnc(C3CCCCC3)cn2)c2ccccc2)cc(F)c1F] | 1.42 ± 0.14 |
| 19 | H237 | 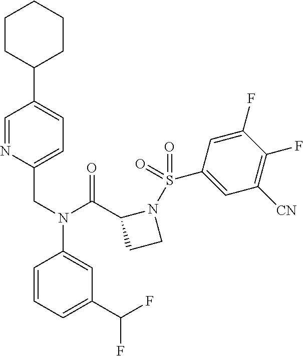 [SMILES: N#Cc1cc(S(=O)(=O)N2CC[C@@H]2C(=O)N(Cc2ccc(C3CCCCC3)cn2)c2cccc(C(F)F)c2)cc(F)c1F] | 2.58 ± 0.07 |
| 20 | H244 | 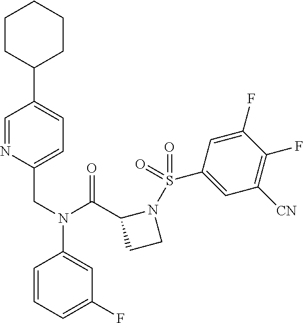 [SMILES: N#Cc1cc(S(=O)(=O)N2CC[C@@H]2C(=O)N(Cc2ccc(C3CCCCC3)cn2)c2cccc(F)c2)cc(F)c1F] | 0.978 ± 0.203 |
| 21 | H245 | 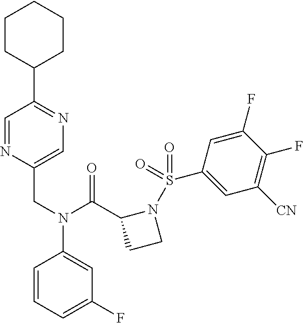 [SMILES: N#Cc1cc(S(=O)(=O)N2CC[C@@H]2C(=O)N(Cc2cnc(C3CCCCC3)cn2)c2cccc(F)c2)cc(F)c1F] | n/a |
| 22 | H205 | 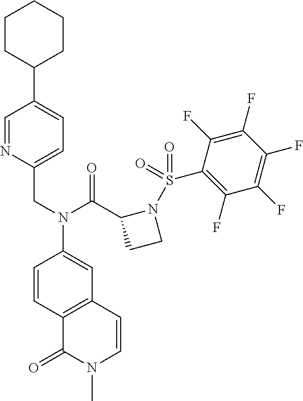 [SMILES: Cn1ccc2cc(N(Cc3ccc(C4CCCCC4)cn3)C(=O)[C@H]3CCN3S(=O)(=O)c3c(F)c(F)c(F)c(F)c3F)ccc2c1=O] | 0.866 ± 0.085 |
| 23 | H206 | 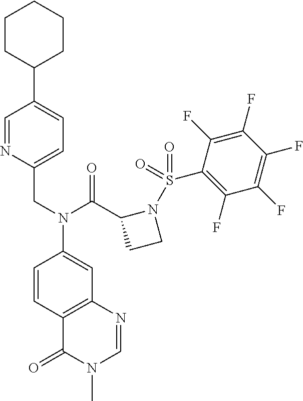 [SMILES: Cn1cnc2cc(N(Cc3ccc(C4CCCCC4)cn3)C(=O)[C@H]3CCN3S(=O)(=O)c3c(F)c(F)c(F)c(F)c3F)ccc2c1=O] | 1.15 ± 0.04 |
| 24 | H207 | 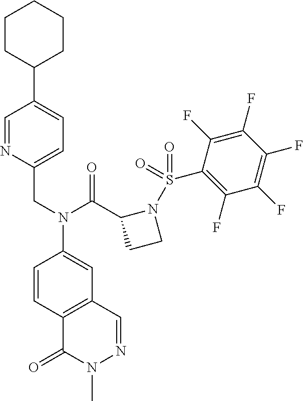 [SMILES: Cn1ncc2cc(N(Cc3ccc(C4CCCCC4)cn3)C(=O)[C@H]3CCN3S(=O)(=O)c3c(F)c(F)c(F)c(F)c3F)ccc2c1=O] | 0.961 ± 0.009 |
| 25 | H208 | 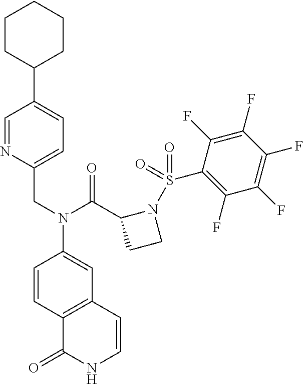 [SMILES: O=C([C@H]1CCN1S(=O)(=O)c1c(F)c(F)c(F)c(F)c1F)N(Cc1ccc(C2CCCCC2)cn1)c1ccc2c(=O)[nH]ccc2c1] | 0.788 ± 0.039 |
| 26 | H213 | 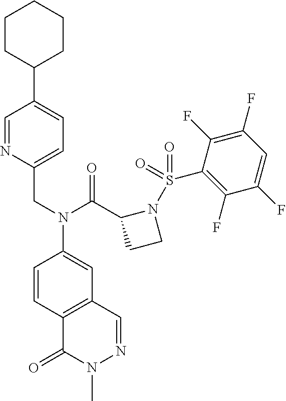 [SMILES: Cn1ncc2cc(N(Cc3ccc(C4CCCCC4)cn3)C(=O)[C@H]3CCN3S(=O)(=O)c3c(F)c(F)cc(F)c3F)ccc2c1=O] | >50 |
| 27 | H214 | 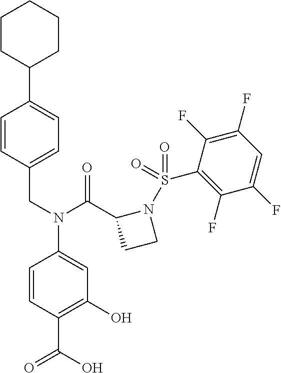 [SMILES: O=C(O)c1ccc(N(Cc2ccc(C3CCCCC3)cc2)C(=O)[C@H]2CCN2S(=O)(=O)c2c(F)c(F)cc(F)c2F)cc1O] | >50 |
| 28 | H215 | 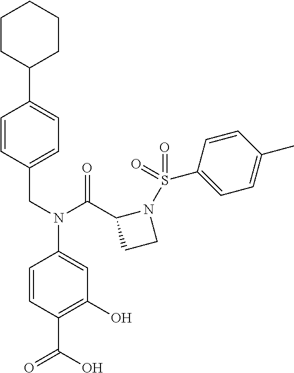 [SMILES: Cc1ccc(S(=O)(=O)N2CC[C@@H]2C(=O)N(Cc2ccc(C3CCCCC3)cc2)c2ccc(C(=O)O)c(O)c2)cc1] | >80 |
| 29 | H218 | 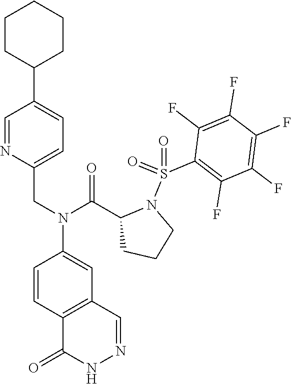 [SMILES: O=C([C@H]1CCCN1S(=O)(=O)c1c(F)c(F)c(F)c(F)c1F)N(Cc1ccc(C2CCCCC2)cn1)c1ccc2c(=O)[nH]ncc2c1] | 5.9 ± 1.8 |
| 30 | H219 | 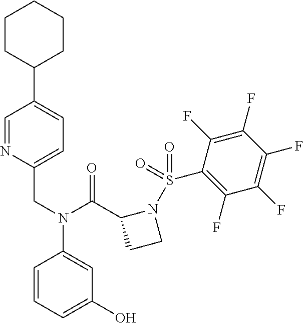 [SMILES: O=C([C@H]1CCN1S(=O)(=O)c1c(F)c(F)c(F)c(F)c1F)N(Cc1ccc(C2CCCCC2)cn1)c1cccc(O)c1] | 1.04 ± 0.07 |
| 31 | H220 | 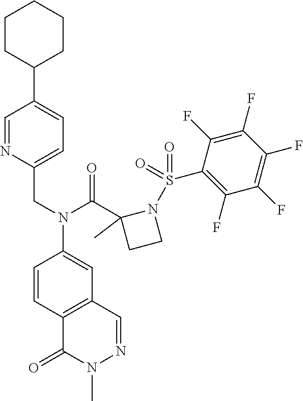 [SMILES: Cn1ncc2cc(N(Cc3ccc(C4CCCCC4)cn3)C(=O)C3(C)CCN3S(=O)(=O)c3c(F)c(F)c(F)c(F)c3F)ccc2c1=O] | >80 |
| 32 | H221 | 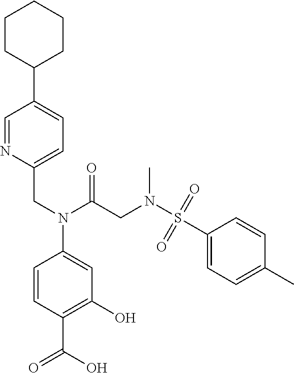 [SMILES: Cc1ccc(S(=O)(=O)N(C)CC(=O)N(Cc2ccc(C3CCCCC3)cn2)c2ccc(C(=O)O)c(O)c2)cc1] | 63.7 ± 3.9 |
| 33 | H224 | 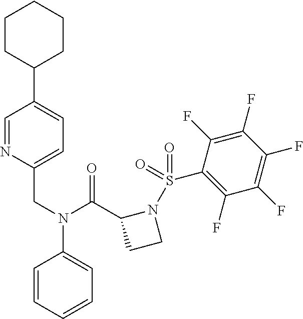 [SMILES: O=C([C@H]1CCN1S(=O)(=O)c1c(F)c(F)c(F)c(F)c1F)N(Cc1ccc(C2CCCCC2)cn1)c1ccccc1] | 1.08 ± 0.05 |
| 34 | H228 | 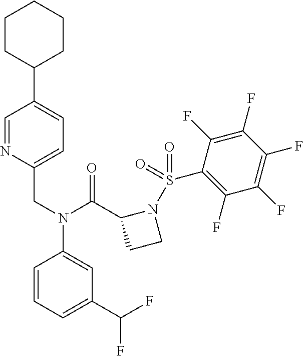 [SMILES: O=C([C@H]1CCN1S(=O)(=O)c1c(F)c(F)c(F)c(F)c1F)N(Cc1ccc(C2CCCCC2)cn1)c1cccc(C(F)F)c1] | 1.78 ± 0.28 |
| 35 | H229 | 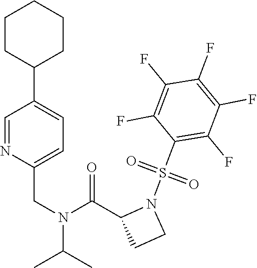 [SMILES: CC(C)N(Cc1ccc(C2CCCCC2)cn1)C(=O)[C@H]1CCN1S(=O)(=O)c1c(F)c(F)c(F)c(F)c1F] | >10 |
| 36 | H233 | 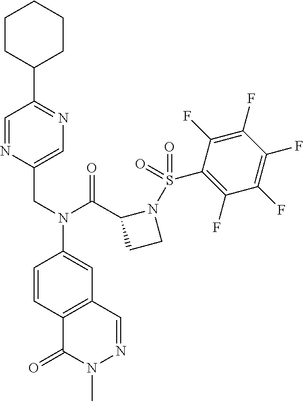 [SMILES: Cn1ncc2cc(N(Cc3cnc(C4CCCCC4)cn3)C(=O)[C@H]3CCN3S(=O)(=O)c3c(F)c(F)c(F)c(F)c3F)ccc2c1=O] | 4.32 ± 1.02 |
| 37 | H239 | 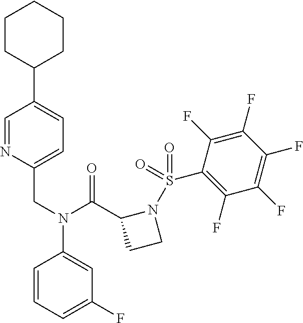 [SMILES: O=C([C@H]1CCN1S(=O)(=O)c1c(F)c(F)c(F)c(F)c1F)N(Cc1ccc(C2CCCCC2)cn1)c1cccc(F)c1] | >4 |
| 38 | H246 | 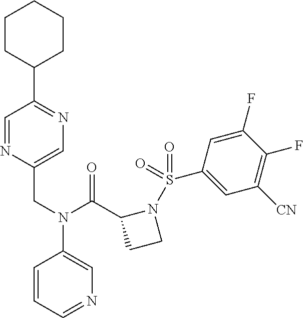 [SMILES: N#Cc1cc(S(=O)(=O)N2CC[C@@H]2C(=O)N(Cc2cnc(C3CCCCC3)cn2)c2cccnc2)cc(F)c1F] | 4.9 ± 1.2 |
| 39 | H250 | 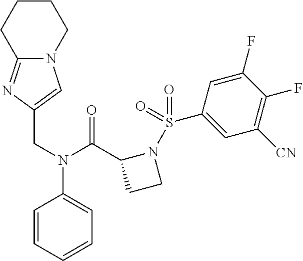 [SMILES: N#Cc1cc(S(=O)(=O)N2CC[C@@H]2C(=O)N(Cc2cn3c(n2)CCCC3)c2ccccc2)cc(F)c1F] | 0.900 ± 0.386 |
| 40 | H253 | 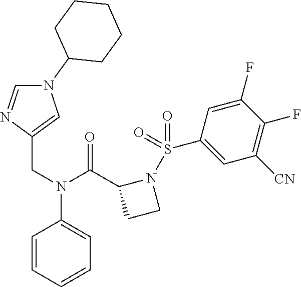 [SMILES: N#Cc1cc(S(=O)(=O)N2CC[C@@H]2C(=O)N(Cc2cn(C3CCCCC3)cn2)c2ccccc2)cc(F)c1F] | 1.5 ± 0.6 |
| 41 | H257 | 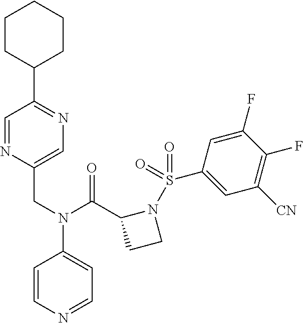 [SMILES: N#Cc1cc(S(=O)(=O)N2CC[C@@H]2C(=O)N(Cc2cnc(C3CCCCC3)cn2)c2ccncc2)cc(F)c1F] | 4.5 ± 0.6 |
| 42 | H258 | 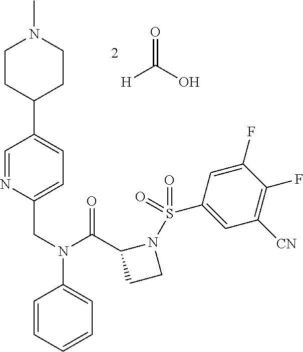 [SMILES: CN1CCC(c2ccc(CN(C(=O)[C@H]3CCN3S(=O)(=O)c3cc(F)c(F)c(C#N)c3)c3ccccc3)nc2)CC1 ; O=CO] | >10 |
| 43 | H260 | 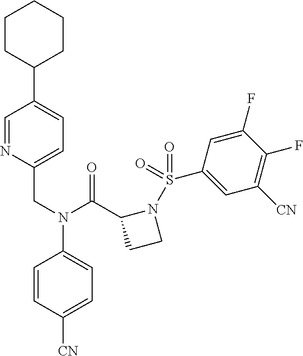 [SMILES: N#Cc1ccc(N(Cc2ccc(C3CCCCC3)cn2)C(=O)[C@H]2CCN2S(=O)(=O)c2cc(F)c(F)c(C#N)c2)cc1] | 2.4 ± 0.4 |
| 44 | H266 | 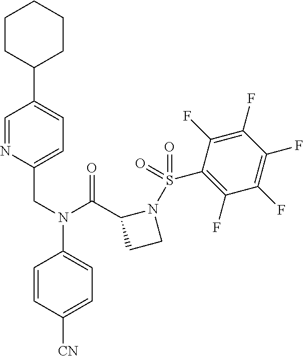 [SMILES: N#Cc1ccc(N(Cc2ccc(C3CCCCC3)cn2)C(=O)[C@H]2CCN2S(=O)(=O)c2c(F)c(F)c(F)c(F)c2F)cc1] | 10.6 ± 0.9 |
| 45 | H267 | 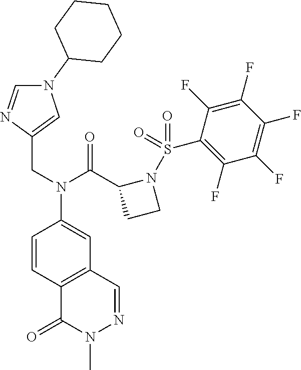 [SMILES: Cn1ncc2cc(N(Cc3cn(C4CCCCC4)cn3)C(=O)[C@H]3CCN3S(=O)(=O)c3c(F)c(F)c(F)c(F)c3F)ccc2c1=O] | 8.7 ± 0.6 |
| 46 | H272 | 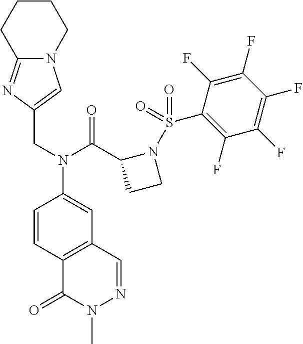 [SMILES: Cn1ncc2cc(N(Cc3cn4c(n3)CCCC4)C(=O)[C@H]3CCN3S(=O)(=O)c3c(F)c(F)c(F)c(F)c3F)ccc2c1=O] | 15.0 ± 0.4 |
| 47 | H273 | 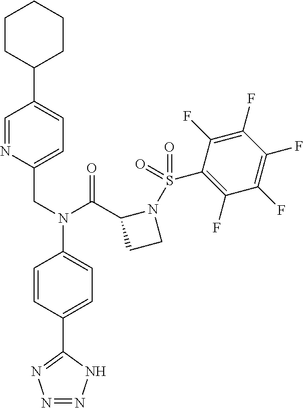 [SMILES: O=C([C@H]1CCN1S(=O)(=O)c1c(F)c(F)c(F)c(F)c1F)N(Cc1ccc(C2CCCCC2)cn1)c1ccc(-c2nnn[nH]2)cc1] | 2.7 ± 0.1 |
| 48 | H274 | 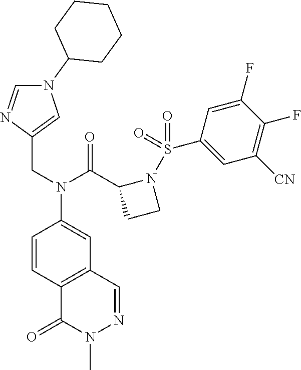 [SMILES: Cn1ncc2cc(N(Cc3cn(C4CCCCC4)cn3)C(=O)[C@H]3CCN3S(=O)(=O)c3cc(F)c(F)c(C#N)c3)ccc2c1=O] | 3.1 ± 0.2 |
| 49 | H277 | 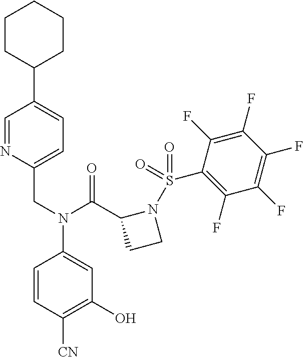 [SMILES: N#Cc1ccc(N(Cc2ccc(C3CCCCC3)cn2)C(=O)[C@H]2CCN2S(=O)(=O)c2c(F)c(F)c(F)c(F)c2F)cc1O] | 0.795 ± 0.136 |
| 50 | H285 | 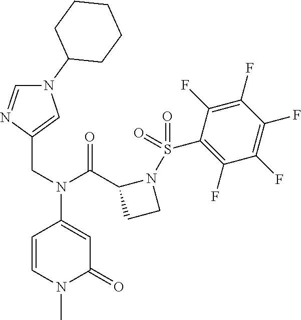 [SMILES: Cn1ccc(N(Cc2cn(C3CCCCC3)cn2)C(=O)[C@H]2CCN2S(=O)(=O)c2c(F)c(F)c(F)c(F)c2F)cc1=O] | 1.8 ± 0.1 |
| 51 | H286 | 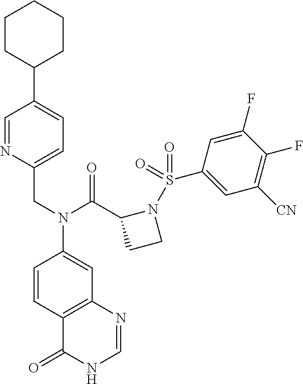 [SMILES: N#Cc1cc(S(=O)(=O)N2CC[C@@H]2C(=O)N(Cc2ccc(C3CCCCC3)cn2)c2ccc3c(=O)[nH]cnc3c2)cc(F)c1F] | 0.657 ± 0.187 |
| 52 | H290 | 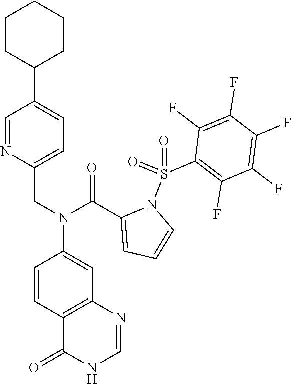 [SMILES: O=C(c1cccn1S(=O)(=O)c1c(F)c(F)c(F)c(F)c1F)N(Cc1ccc(C2CCCCC2)cn1)c1ccc2c(=O)[nH]cnc2c1] | 2.5 ± 0.4 |
| 53 | H291 | 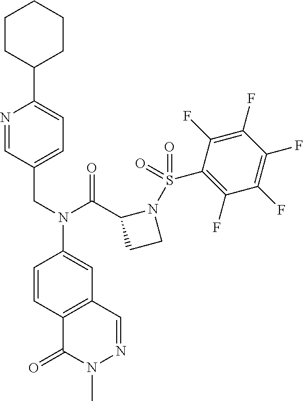 [SMILES: Cn1ncc2cc(N(Cc3ccc(C4CCCCC4)nc3)C(=O)[C@H]3CCN3S(=O)(=O)c3c(F)c(F)c(F)c(F)c3F)ccc2c1=O] | >4 |
| 54 | H293 | 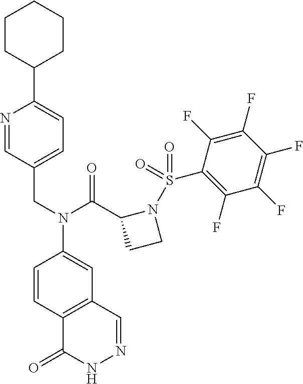 [SMILES: O=C([C@H]1CCN1S(=O)(=O)c1c(F)c(F)c(F)c(F)c1F)N(Cc1ccc(C2CCCCC2)nc1)c1ccc2c(=O)[nH]ncc2c1] | 4.38 ± 0.63 |
| 55 | H295 | 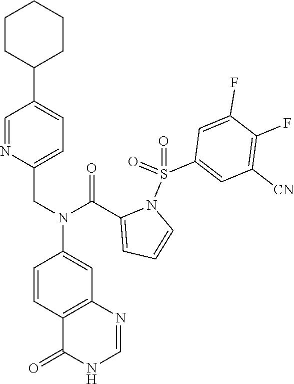 [SMILES: N#Cc1cc(S(=O)(=O)n2cccc2C(=O)N(Cc2ccc(C3CCCCC3)cn2)c2ccc3c(=O)[nH]cnc3c2)cc(F)c1F] | 1.58 ± 0.49 |
| 56 | H289 | 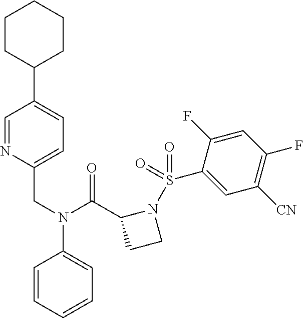 [SMILES: N#Cc1cc(S(=O)(=O)N2CC[C@@H]2C(=O)N(Cc2ccc(C3CCCCC3)cn2)c2ccccc2)c(F)cc1F] | >4 |
| 57 | H275 | 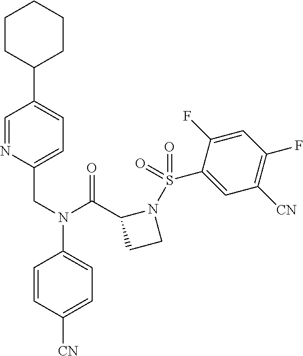 [SMILES: N#Cc1ccc(N(Cc2ccc(C3CCCCC3)cn2)C(=O)[C@H]2CCN2S(=O)(=O)c2cc(C#N)c(F)cc2F)cc1] | 2.2 ± 0.5 |
| 58 | H270 | 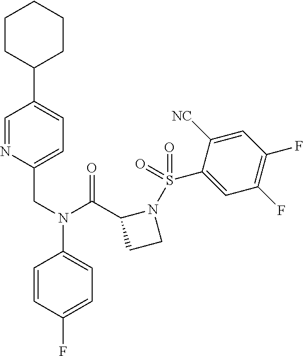 [SMILES: N#Cc1cc(F)c(F)cc1S(=O)(=O)N1CC[C@@H]1C(=O)N(Cc1ccc(C2CCCCC2)cn1)c1ccc(F)cc1] | >20 |
| 59 | H265 | 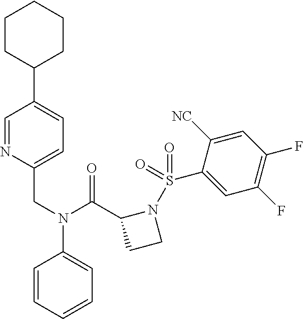 [SMILES: N#Cc1cc(F)c(F)cc1S(=O)(=O)N1CC[C@@H]1C(=O)N(Cc1ccc(C2CCCCC2)cn1)c1ccccc1] | >30 |
| 60 | H287 | 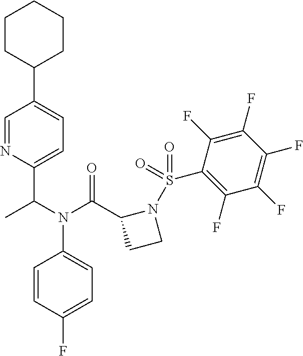 [SMILES: CC(c1ccc(C2CCCCC2)cn1)N(C(=O)[C@H]1CCN1S(=O)(=O)c1c(F)c(F)c(F)c(F)c1F)c1ccc(F)cc1] | 2.9 ± 0.1 |
| 61 | H247 | 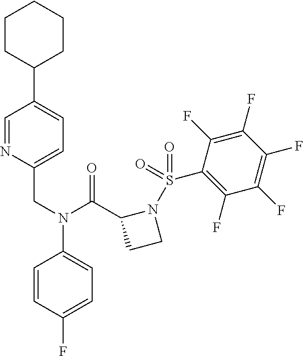 [SMILES: O=C([C@H]1CCN1S(=O)(=O)c1c(F)c(F)c(F)c(F)c1F)N(Cc1ccc(C2CCCCC2)cn1)c1ccc(F)cc1] | 4.7 ± 0.5 |
| 62 | H254 | 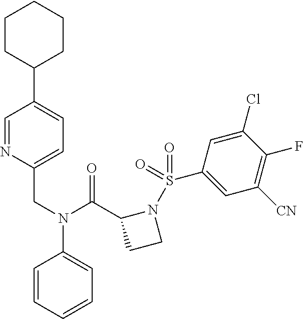 [SMILES: N#Cc1cc(S(=O)(=O)N2CC[C@@H]2C(=O)N(Cc2ccc(C3CCCCC3)cn2)c2ccccc2)cc(Cl)c1F] | 2.1 ± 0.1 |
| 63 | H261 | 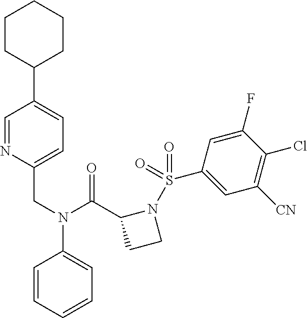 [SMILES: N#Cc1cc(S(=O)(=O)N2CC[C@@H]2C(=O)N(Cc2ccc(C3CCCCC3)cn2)c2ccccc2)cc(F)c1Cl] | 87.4 ± 5.6 |
| 64 | H249 | 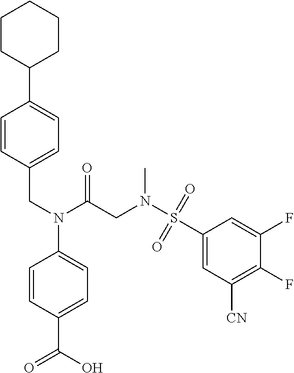 [SMILES: CN(CC(=O)N(Cc1ccc(C2CCCCC2)cc1)c1ccc(C(=O)O)cc1)S(=O)(=O)c1cc(F)c(F)c(C#N)c1] | >4 |
| 65 | H255 | 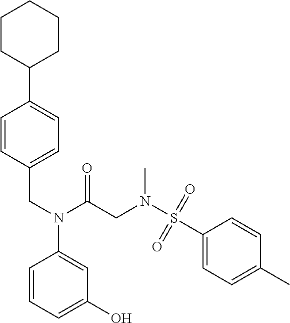 [SMILES: Cc1ccc(S(=O)(=O)N(C)CC(=O)N(Cc2ccc(C3CCCCC3)cc2)c2cccc(O)c2)cc1] | 164 ± 3 |
| 66 | H256 | 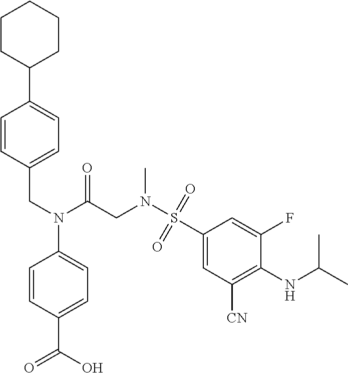 [SMILES: CC(C)Nc1c(F)cc(S(=O)(=O)N(C)CC(=O)N(Cc2ccc(C3CCCCC3)cc2)c2ccc(C(=O)O)cc2)cc1C#N] | >30 |
| 67 | H259 | 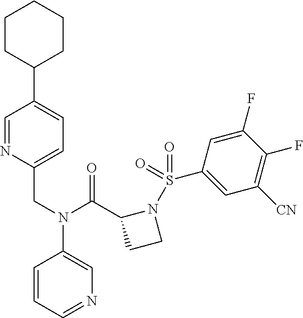 [SMILES: N#Cc1cc(S(=O)(=O)N2CC[C@@H]2C(=O)N(Cc2ccc(C3CCCCC3)cn2)c2cccnc2)cc(F)c1F] | 6.5 ± 0.1 |
| 68 | H262 | 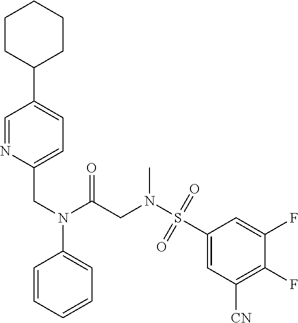 [SMILES: CN(CC(=O)N(Cc1ccc(C2CCCCC2)cn1)c1ccccc1)S(=O)(=O)c1cc(F)c(F)c(C#N)c1] | >20 |
| 69 | H263 | 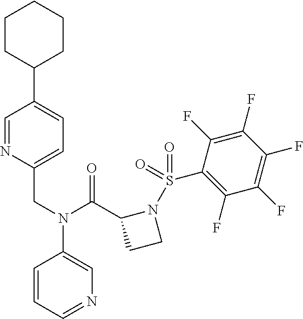 [SMILES: O=C([C@H]1CCN1S(=O)(=O)c1c(F)c(F)c(F)c(F)c1F)N(Cc1ccc(C2CCCCC2)cn1)c1cccnc1] | >10 |
| 70 | H264 | 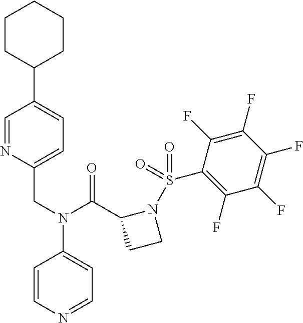 [SMILES: O=C([C@H]1CCN1S(=O)(=O)c1c(F)c(F)c(F)c(F)c1F)N(Cc1ccc(C2CCCCC2)cn1)c1ccncc1] | 4.8 ± 1.0 |
| 71 | H268 | 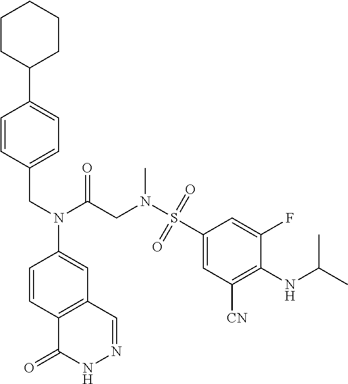 [SMILES: CC(C)Nc1c(F)cc(S(=O)(=O)N(C)CC(=O)N(Cc2ccc(C3CCCCC3)cc2)c2ccc3c(=O)[nH]ncc3c2)cc1C#N] | >30 |
| 72 | H269 | 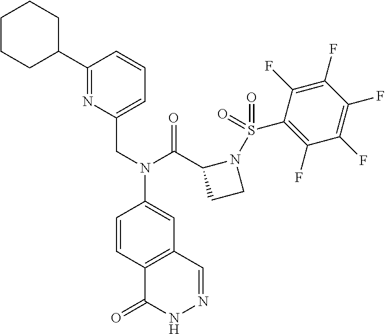 [SMILES: O=C([C@H]1CCN1S(=O)(=O)c1c(F)c(F)c(F)c(F)c1F)N(Cc1cccc(C2CCCCC2)n1)c1ccc2c(=O)[nH]ncc2c1] | 3.4 ± 0.2 |
| 73 | H271 | 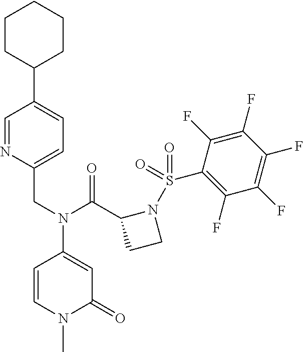 [SMILES: Cn1ccc(N(Cc2ccc(C3CCCCC3)cn2)C(=O)[C@H]2CCN2S(=O)(=O)c2c(F)c(F)c(F)c(F)c2F)cc1=O] | 5.8 ± 2.9 |
| 74 | H276 | 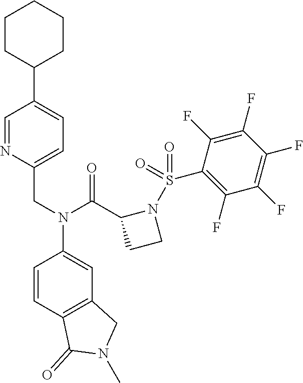 [SMILES: CN1Cc2cc(N(Cc3ccc(C4CCCCC4)cn3)C(=O)[C@H]3CCN3S(=O)(=O)c3c(F)c(F)c(F)c(F)c3F)ccc2C1=O] | 4.9 ± 0.5 |
| 75 | H280 | 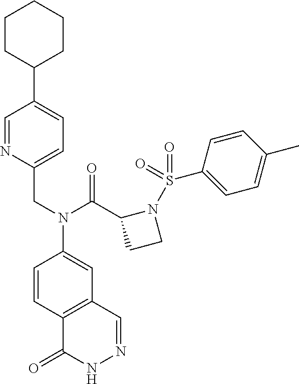 [SMILES: Cc1ccc(S(=O)(=O)N2CC[C@@H]2C(=O)N(Cc2ccc(C3CCCCC3)cn2)c2ccc3c(=O)[nH]ncc3c2)cc1] | >10 |
| 76 | H281 | 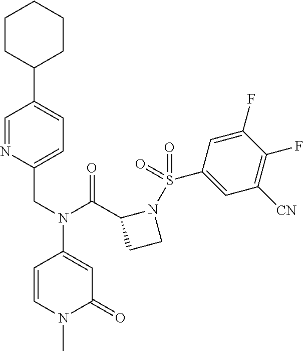 [SMILES: Cn1ccc(N(Cc2ccc(C3CCCCC3)cn2)C(=O)[C@H]2CCN2S(=O)(=O)c2cc(F)c(F)c(C#N)c2)cc1=O] | 1.14 ± 0.23 |
| 77 | H283 | 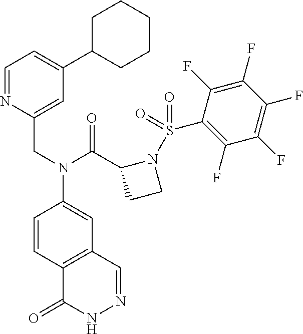 [SMILES: O=C([C@H]1CCN1S(=O)(=O)c1c(F)c(F)c(F)c(F)c1F)N(Cc1cc(C2CCCCC2)ccn1)c1ccc2c(=O)[nH]ncc2c1] | 0.733 ± 0.192 |
| 78 | H284 | 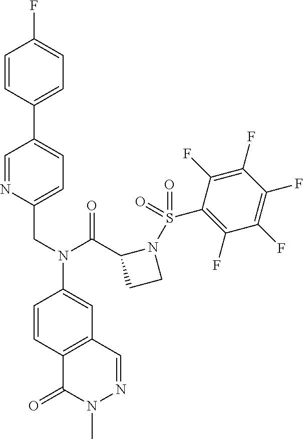 [SMILES: Cn1ncc2cc(N(Cc3ccc(-c4ccc(F)cc4)cn3)C(=O)[C@H]3CCN3S(=O)(=O)c3c(F)c(F)c(F)c(F)c3F)ccc2c1=O] | 2.2 ± 0.9 |
| 79 | H288 | 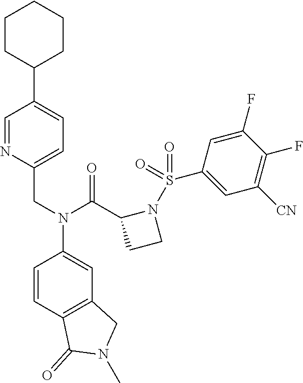 [SMILES: CN1Cc2cc(N(Cc3ccc(C4CCCCC4)cn3)C(=O)[C@H]3CCN3S(=O)(=O)c3cc(F)c(F)c(C#N)c3)ccc2C1=O] | 0.972 ± 0.054 |
| 80 | H279 | 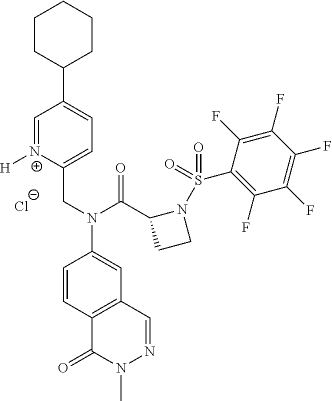 [SMILES: Cn1ncc2cc(N(Cc3ccc(C4CCCCC4)c[nH+]3)C(=O)[C@H]3CCN3S(=O)(=O)c3c(F)c(F)c(F)c(F)c3F)ccc2c1=O ; [Cl-]] | 0.961 ± 0.009 |
| 81 | H278 | 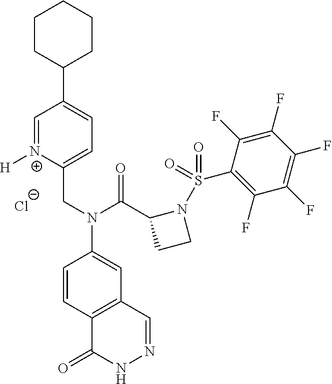 [SMILES: O=C([C@H]1CCN1S(=O)(=O)c1c(F)c(F)c(F)c(F)c1F)N(Cc1ccc(C2CCCCC2)c[nH+]1)c1ccc2c(=O)[nH]ncc2c1 ; [Cl-]] | 0.663 ± 0.102 |
| 82 | H282 | 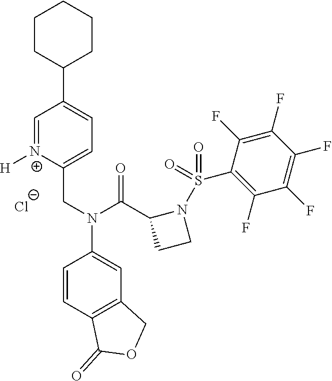 [SMILES: O=C1OCc2cc(N(Cc3ccc(C4CCCCC4)c[nH+]3)C(=O)[C@H]3CCN3S(=O)(=O)c3c(F)c(F)c(F)c(F)c3F)ccc21 ; [Cl-]] | 1.791 ± 0.107 |
| 83 | H248 | 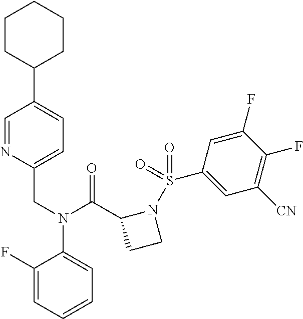 [SMILES: N#Cc1cc(S(=O)(=O)N2CC[C@@H]2C(=O)N(Cc2ccc(C3CCCCC3)cn2)c2ccccc2F)cc(F)c1F] | 2.2 ± 0.2 |
| 84 | H251 | 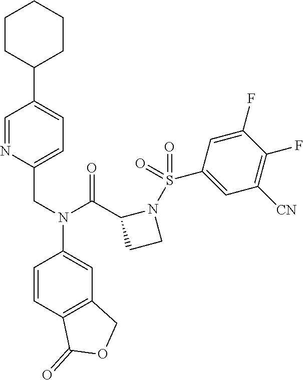 [SMILES: N#Cc1cc(S(=O)(=O)N2CC[C@@H]2C(=O)N(Cc2ccc(C3CCCCC3)cn2)c2ccc3c(c2)COC3=O)cc(F)c1F] | 3.2 ± 0.1 |
| 85 | H252 | 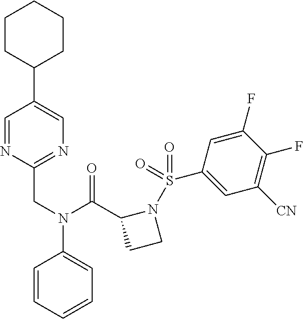 [SMILES: N#Cc1cc(S(=O)(=O)N2CC[C@@H]2C(=O)N(Cc2ncc(C3CCCCC3)cn2)c2ccccc2)cc(F)c1F] | 3.5 ± 0.5 |
| 86 | H296 | 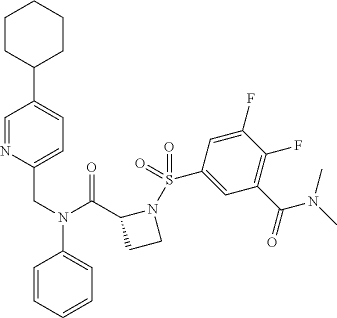 [SMILES: CN(C)C(=O)c1cc(S(=O)(=O)N2CC[C@@H]2C(=O)N(Cc2ccc(C3CCCCC3)cn2)c2ccccc2)cc(F)c1F] | >20 |
| 87 | H292 | 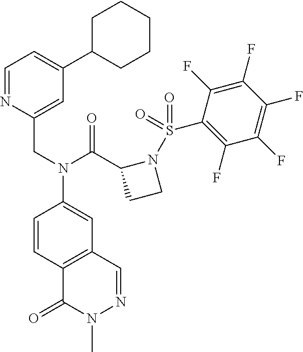 [SMILES: Cn1ncc2cc(N(Cc3cc(C4CCCCC4)ccn3)C(=O)[C@H]3CCN3S(=O)(=O)c3c(F)c(F)c(F)c(F)c3F)ccc2c1=O] | 2.89 ± 0.35 |
| 88 | H294 | 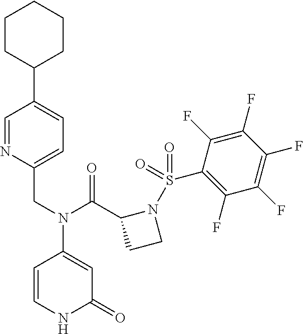 [SMILES: O=C([C@H]1CCN1S(=O)(=O)c1c(F)c(F)c(F)c(F)c1F)N(Cc1ccc(C2CCCCC2)cn1)c1cc[nH]c(=O)c1] | 9.6 ± 1.2 |

Table 1. Stat3 DNA-binding activity in vitro of aryl sulfonamido compounds described herein, including their corresponding referenced names (e.g., H203), as measured by electrophoretic mobility shift assay (EMSA). The inhibition metrics are presented in units of micromolar (μM).

In addition, EMSA values were measured for compounds H169, H172, and H182, the structures and values of which are provided below:

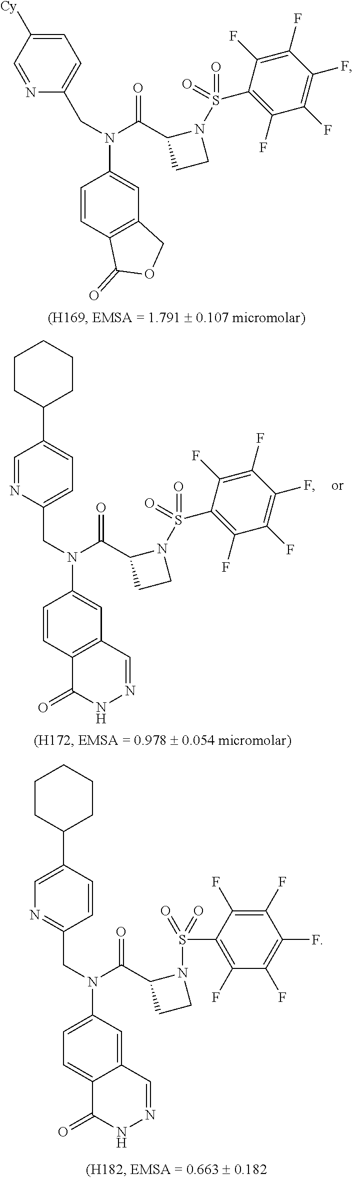

Aryl Sulfonamide Compounds Inhibit Constitutive Stat3 Activation and Function in Cancer Cells, with Minimal Effects on Other Signaling Proteins.

Aryl sulfonamido azetidine compounds were investigated for their inhibitory effects of the active analogs on intracellular Stat3 signaling. Human breast cancer cells, MDA-MB-231, and MDA-MB-468 were treated with 0.5, 1, 2, 3, 4, 5, and 10 μM of aryl sulfonamido azetidine compounds H172 and H182 for 0-72 h. Nuclear extracts are prepared and subjected to EMSA analysis for Stat3 DNA-binding activity. Results demonstrated that Stat3 DNA-binding activity, as shown in FIGS. 3A-3D, including FIGS. 3A, 3B, 3C, and 3D. Stat3 DNA-binding activity in MDA-MB-231 cells, is inhibited by the compounds following treatment as measured periodically up to 72 h.

### Aryl Sulfonamide Compounds Decrease the Growth of Human Cancer Cells Harboring Persistently Active Stat3

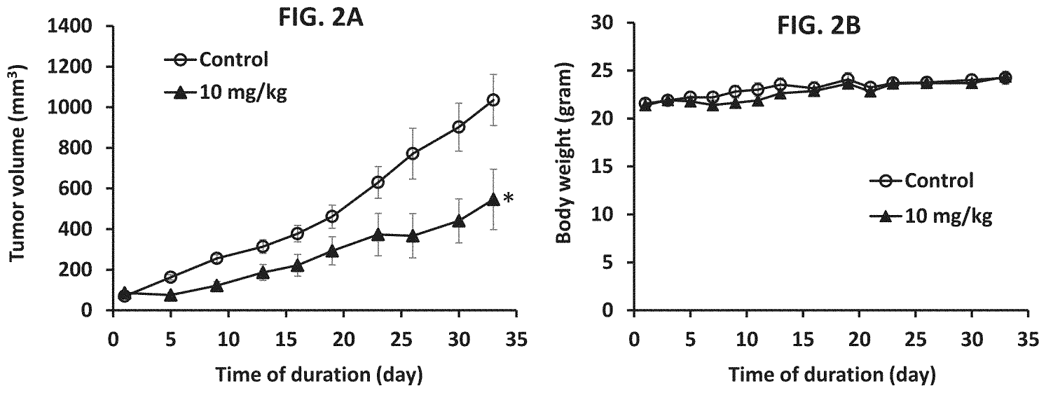

Constitutive Stat3 activity promotes tumor cell growth and proliferation and survival (Darnell J E (2005) Nat Med. 11:595-6; Bowman T, et al., (2000) Oncogene 19:2474-2488). An aryl sulfonamido azetidine compound, H182, was tested against tumor cells harboring constitutively-active Stat3. Compound H182 suppressed the human breast cancer line MDA-MB-231 cell growth in a time- or dose-dependent manner, as measured by trypan blue exclusion-phase contrast microscopy, with IC50 found to be lower than 10 μM against MDA-MB-231, as shown in FIGS. 2A-2B.

### Example 90. Synergistic Effects of Compounds of the Invention with Chemotherapeutic Agent

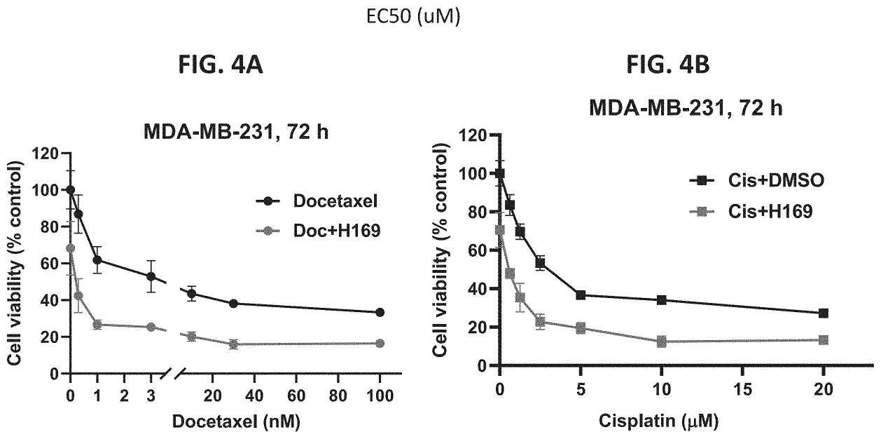

The synergistic effects of using an aryl sulfonamido azetidine compound (H169) and clinically used chemotherapy drugs on cell viability is shown in FIGS. 4A-4B. Human triple-negative breast cancer cell line MDA-MB-231 (5000 cells/well) were seeded in 96-well plates. On the next day, cells were first treated with H169 at 1 μM or DMSO (control, 0.5% (v/v)) for 6 hours prior to treatment with an increasing concentrations of cisplatin (which interferes with DNA repair mechanisms, causing DNA damage, and subsequently inducing apoptosis in cancer cells) or docetaxel (a tubulin modulator which interferes with cancer cell metastasis), and incubated for a total of 72 hours. Cell proliferation and viability were measured by CyQuant NF Cell Proliferation Assay kit (ThermoFisher Scientific). Cell viability was normalized to the percentage of the control groups. FIG. 4A shows the synergistic effect of docetaxel and H169, wherein the combination of docetaxel with H169 resulted in a cancer cell death than docetaxel alone. FIG. 4B shows the synergistic effect of cisplatin and H169, wherein the combination of docetaxel with H169 resulted in a cancer cell death than cisplatin alone. The synergistic effects are observed for several different orthogonal modes of action—the compounds of the present invention being Stat3 inhibitors, and cisplatin—DNA repair inhibitor, or docetaxel—a metastasis inhibitor. Therefore, the results indicate that a synergy can be achieved using the compounds of the invention with a variety of orthogonal mechanism of action chemotherapeutic agents. In addition, the inventors have observed that the combination of docetaxel and a compound of the invention results in a higher rate of tumor volume reduction than docetaxel alone or the compound alone, further establishing the synergistic effects of the methods of the present invention.

### Example 91: Reduction in Tumor Volume Growth Using Aryl Sulfonamido Compounds

An aryl sulfonamido compound, H182, was found to reduce the tumor growth rate, while maintaining subject body weight, throughout the course of treatment. As shown in FIGS. 2A-2B, the in vivo efficacy of an aryl sulfonamido azetidine compound (H182) was observed on the growth of subcutaneous MDA-MB-231 tumor xenografts. FIG. 2A shows the mouse model tumor volume as a function of time dosed (10 mg/kg every other day) treatment compared to vehicle control. (5 mice per group). The subjects were administered intraperitoneally. The results demonstrate that aryl sulfonamido azetidine compounds (H182) are effective in reducing the tumor growth volume relative to vehicle control, and thereby treat a subject having cancer. FIG. 2B shows the body weight of treated vs. untreated mice were equivalent throughout the treatment regime, indicating that the aryl sulfonamido compounds did not result in weight loss of the subjects.

### Example 92: Inhibiting Stat3 with Minimal Impact on Tubulin Activity Using Aryl Sulfonamido Compounds

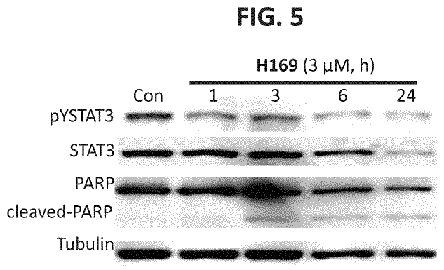

An aryl sulfonamido azetidine compounds, H169, was found to induce the apoptosis of human breast cancer cells. Human breast cancer cells, MDA-MB-231, were treated in culture with 3 micromolar concentration of H169 for 0-24 hours, whole-cell lysates prepared, and sample of equal total protein were subjected to SDS/PAGE-Western blotting analysis probing for pYSTAT3, STAT3, full-length PARP, cleaved PARP, and tubulin. FIG. 5 shows the positions of proteins in gel are shown; control (“Con”, no H169) lane represents whole-cell lysates prepared from 0.05% DMSO-treated cells. Data are representative of 2 independent determinations. The data show that while STAT3 is significantly inhibited in the treated cells after 24 hr exposure, the tubulin and PARP concentrations are not affected much, indicating the selective STAT3 inhibition of the aryl sulfonamido azetidine compound H169.

### Example 93: Inhibition of Multiple Cancer Cell Types Using Representative Compounds of the Invention

To further establish the anti-cancer properties of the aryl sulfonamido compounds described herein, the cell proliferation and viability assay described in Example 89 is performed against a wide variety of cancer cell types wherein intracellular Stat3 signaling is thereby modulated. STAT3 activation is clearly a factor linked to bad prognosis in patients with lung cancer, liver cancer, renal cell carcinoma (RCC) and gliomas (Igelmann, et al., Cancers (Basel). 2019 October; 11(10): 1428). The following cell lines are used to demonstrate the potency of the anti-cancer properties of the compounds of the invention using the methods described herein to the aforementioned cancer cell types: Normal cells (control): Hepatocytes, HUVECs, NHDF; Breast cancer: MCF-7, MDA-MB-231, ZR-75; Head and neck cancer: KB; Skin cancer: A431; Stomach cancer: KATO III; Liver cancer: Hep 3B, HepG2; Kidney cancer: A-498, ACHN; Melanoma: SK-MEL-5; Lung cancer: A549, NCI-H460, PC-6; Ovarian cancer: OVCAR4, OVCAR3; Prostate cancer: PC-3; Brain cancer (glioma): U-87 MG, T98. Results are expected to show inhibition of Stat3 DNA-binding activity for a variety of cancer cell types, suggesting the described compounds herein can be used to treat a variety of cancer types.

Patents, patent applications, publications, scientific articles, books, web sites, and other documents and materials referenced or mentioned herein are indicative of the levels of skill of those skilled in the art to which the inventions pertain. Each such referenced document and material is hereby incorporated by reference to the same extent as if it had been incorporated by reference in its entirety individually or set forth or reprinted herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, and the claims referred to above, including, but not limited to, any original claims.

The inventions have been described broadly and generically herein. Each of the narrower species and subgeneric groupings falling within the generic disclosure also form part of these inventions. This includes the generic description of each invention which hereby include, including any claims thereto, a proviso or negative limitation removing, or optionally allowing the removal of, any subject matter from the genus, regardless of whether or not the excised materials, or options, were specifically recited or identified in haec verba herein, and all such variations form a part of the original written description of the inventions. In addition, where features, or aspects, of an invention are described in terms of a Markush group, the invention shall be understood thereby to be described in terms of each and every, and any, individual member or subgroup of members of the Markush group.

The inventions illustratively described and claimed herein can suitably be practiced in the absence of any element or elements, limitation or limitations, not specifically disclosed herein, or described herein, as essential. Thus, for example, the terms “comprising,” “including,” “containing,” “for example,” etc., shall be read expansively and without limitation. The term “including” means “including but not limited to.” The phrase “for example” is not limited to, or by, the items that follow the phrase.

In claiming their inventions, the inventors reserve the right to substitute any transitional phrase with any other transitional phrase, and the inventions shall be understood to include such substituted transitions and form part of the original written description of the inventions. Thus, for example, the term “comprising” may be replaced with either of the transitional phrases “consisting essentially of” or “consisting of.”

The methods and processes illustratively described herein may be suitably practiced in differing orders of steps. They are not necessarily restricted to the orders of steps indicated herein, or in the claims.

Under no circumstances may the patent be interpreted to be limited to the specific examples, or embodiments, or methods, specifically disclosed herein. Under no circumstances may the patent be interpreted to be limited by any statement made by any Examiner, or any other official or employee of the Patent and Trademark Office, unless such statement was specifically, and without qualification or reservation, expressly adopted by Applicants in a responsive writing specifically relating to the application that led to this patent prior to its issuance.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary of, and not intended as limitations on, the scope of the invention. Other objects, aspects, and embodiments will occur to those skilled in the art upon consideration of this specification, and are encompassed within the spirit of the invention as defined by the scope of the claims. Where examples are given, the description shall be construed to include, but not to be limited to, only those examples. It will be readily apparent to one skilled in the art that varying substitutions and modifications may be made to the invention disclosed herein, without departing from the scope and spirit of the invention, and from the description of the inventions, including those illustratively set forth herein, it is manifest that various modifications and equivalents can be used to implement the concepts of the present invention, without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims.

While this invention has been disclosed with reference to specific embodiments, it is apparent that other embodiments and variations of this invention can be devised by those skilled in the art, without departing from the true spirit and scope of the invention. The appended claims include all such embodiments and equivalent variations.

What is claimed is:

1. A compound represented by Formula I: 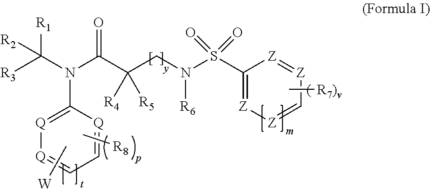
wherein R¹ is selected from aryl or a 5 or 6-membered aryl or heteroaryl, where the heteroatoms are one or more O, N, S(A)₂, where S is sulfur and A is selected from oxygen or an electron pair, the aryl or the 5 or 6-membered heteroaryl are optionally substituted with halogen, CF₃, C₁-C₆ alkyl, C₁-C₆ branched alkyl, aryl (which is optionally further substituted with 1-5 halogens), heteroaryl, C₃-C₇ cycloalkyl, C₃-C₇ cycloalkenyl, three- to six-membered heterocycle, three- to seven-membered saturated heterocycle, fused C₂-C₅ alkylene, where one or more CH₂ groups can be replaced with O, NR⁹, S(A)₂ where S is sulfur and A is selected from oxygen or an electron pair, the aryl or the 5 or 6-membered heteroaryl are optionally substituted naphthalene, optionally substituted indole, benzofuran, benzothiophene, benzimidazole; R² and R³ are independently selected from H or C₁-C₆ alkyl; where R² and R³ together can form a C₃-C₆ cycloalkane ring, where said C₃-C₆ cycloalkane ring can be substituted with 1 or more of C₁-C₆ alkyl, hydroxyl, NR⁹R¹⁰, or C₁-C₆ alkoxy; R⁴ is selected from H, C₁-C₆ alkyl, (CH₂)_{f}NR⁹R¹⁰, (CH₂)_{f}OR⁹, (CH₂)_{f}CO₂R⁹, (CH₂)_{f}CO₂NR⁹R¹⁰; R⁵ is selected from H, C₁-C₆ alkyl, (CH₂)_{f}NR⁹R¹⁰, (CH₂)_{f}OR⁹, (CH₂)_{f}CO2R⁹, (CH₂)_{f}CO₂NR⁹R¹⁰, where R⁴ and R⁵ together can form a C₃-C₆ cycloalkane ring, where said C₃-C₆ cycloalkane ring can be substituted with 1 or more of C₁-C₆ alkyl, hydroxyl, NR⁹R¹⁰, or C₁-C₆ alkoxy, where one or more CH₂ groups can be replaced with O, NR⁹, S(A)₂ where S is sulfur and A is selected from oxygen or an electron pair; R⁶ is selected from C₁-C₆ alkyl; where R⁴ and R⁶ together can form a C₃-C₆ N-heterocycle ring, where said C₃-C₆ N-heterocycle ring can be substituted with 1 or more of C₁-C₆ alkyl, hydroxyl, NR⁹R¹⁰ and where one or more CH₂ groups of said C₁-C₆ alkyl can be replaced with O, NR⁹, S(A)₂ where S is sulfur and A is selected from oxygen or an electron pair; wherein R⁴ and R⁶ together can form an optionally substituted pyrrole ring, wherein one or more CH groups of said pyrrole ring can be replaced with O, N, S(A)₂, where S is sulfur and A is selected from oxygen or an electron pair; where N of said pyrrole ring can be replaced with C and R⁴ and R⁶ together can form aryl, heteroaryl, C₃-C₇ cycloalkyl or C₃-C₇ cycloalkenyl ring, where said aryl, heteroaryl, C₃-C₇ cycloalkyl or C₃-C₇ cycloalkenyl ring can be substituted with 1 or more of C₁-C₆ alkyl, hydroxyl, NR⁹R¹⁰ and where one or more CH₂ groups can be replaced with O, NR⁹, S(A)₂ where S is sulfur and A is selected from oxygen or an electron pair; R⁷ is selected from C₁-C₆ alkyl, halogen, hydroxyl, CN, CF₃, C₁-C₆ alkyl- or dialkyl-amino, C₁-C₆ branched alkyl- or dialkylamino, or C₁-C₆ alkyl- or C₁-C₆ branched alkyl ether; R⁸ is a substitution selected from one or more of H, C₁-C₆ alkyl, C₁-C₆ alkoxy, hydroxyl, halogen, OC(O)CH₃, NR⁹R¹⁰, CN, CF₃, CO₂R⁹, CO₂NR⁹R¹⁰, (CH₂)_{f}NR⁹R¹⁰, (CH₂)_{f}OR⁹, or (CH₂)_{f}CO2R⁹; R⁹ is selected from H or C₁-C₆ alkyl; R¹⁰ is selected from H, C₁-C₆ alkyl; W is selected from H, CO2H, tetrazole, benzyl, C(O)NHOR¹⁰ and CF₂OH; each instance of Q is independently selected from C, CH, N, O, or S; or where Q and R⁸ together can form a 5 or 6 membered lactone or lactam ring, or a heterocyclic ring; each instance of Z is independently selected from C, CH, N, O, or S; p is selected from 0 or 1, y is selected from 0 or 1, f is selected from 0 to 4; t is selected from 0 or 1, v is an integer selected from 1 to 5, m is selected from 0 or 1,
and solvates, hydrates, or pharmaceutically acceptable salts thereof.

2. A compound of claim 1 represented by Formula II: 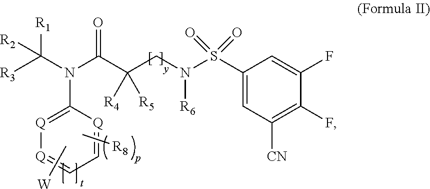
and solvates, hydrates, or pharmaceutically acceptable salts thereof.

3. A compound of claim 2 represented by Formula III: 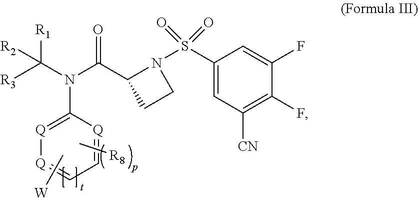
and solvates, hydrates, or pharmaceutically acceptable salts thereof.

4. A compound of claim 1, wherein R⁵ is H.

5. A compound of claim 1, wherein R⁴ and R⁶ together form a C₃-C₆ N-heterocycle ring, where said C₃-C₆ N-heterocycle ring can be substituted with 1 or more of C₁-C₆ alkyl, hydroxyl, NR⁹R¹⁰ and where one or more CH₂ groups of said C₁-C₆ alkyl can be replaced with O, NR⁹, S(A)₂ where S is sulfur and A is selected from oxygen or an electron pair; or where R⁴ and R⁶ form an optionally substituted pyrrole ring, wherein one or more CH groups of said pyrrole ring can be replaced with O, N, S(A)₂, where S is sulfur and A is selected from oxygen or an electron pair; where N of said pyrrole ring can be replaced with C and R⁴ and R⁶ can form aryl, heteroaryl, C₃-C₇ cycloalkyl or C₃-C₇ cycloalkenyl ring, where said aryl, heteroaryl, C₃-C₇ cycloalkyl or C₃-C₇ cycloalkenyl ring can be substituted with 1 or more of C₁-C₆ alkyl, hydroxyl, NR⁹R¹⁰ and where one or more CH₂ groups can be replaced with O, NR⁹, S(A)₂ where S is sulfur and A is selected from oxygen or an electron pair.

6. A compound of claim 1, wherein R⁷ is independently selected from aryl or heteroaryl group where the aryl or heteroaryl group is substituted with 1-5 substituents independently selected from C₁-C₆ alkyl- or dialkyl-amino, C₁-C₆ branched alkyl- or dialkylamino, or C₁-C₆ alkyl- or C₁-C₆ branched alkyl ether, or halogen, or CN.

7. A compound of claim 1, wherein R¹ is aryl or heteroaryl, wherein the aryl or heteroaryl is substituted with aryl which is itself substituted with 1-5 halogens.

8. A compound having the structure of formula IV: 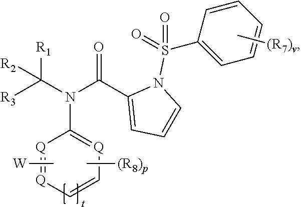
or prodrugs, solvates, hydrates or pharmaceutically acceptable salts thereof.

9. A compound having the structure of Formula V: 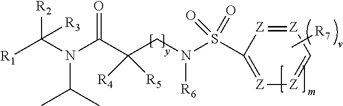
and prodrugs, solvates, hydrates, or pharmaceutically acceptable salts thereof.

10. A compound of claim 1, wherein W is H.

11. A compound having one of the following structures: 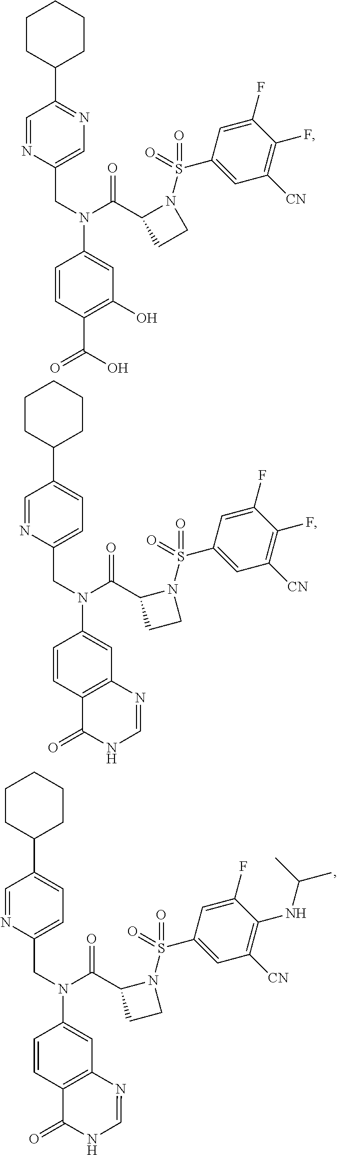 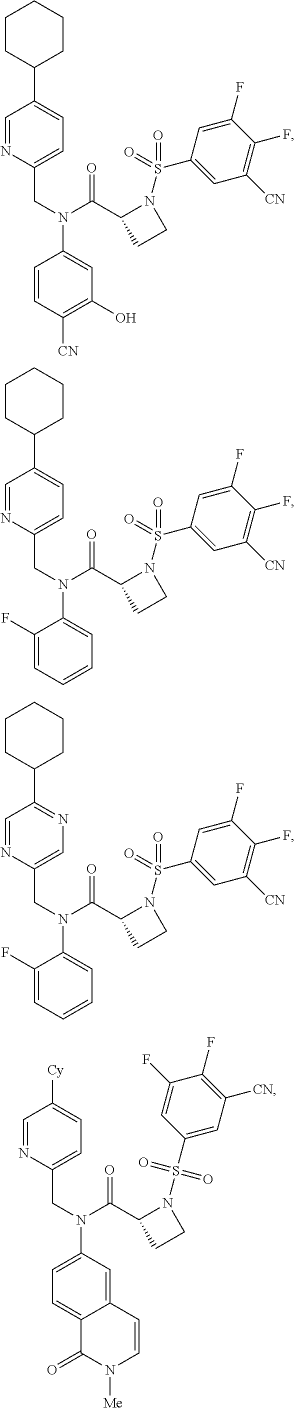 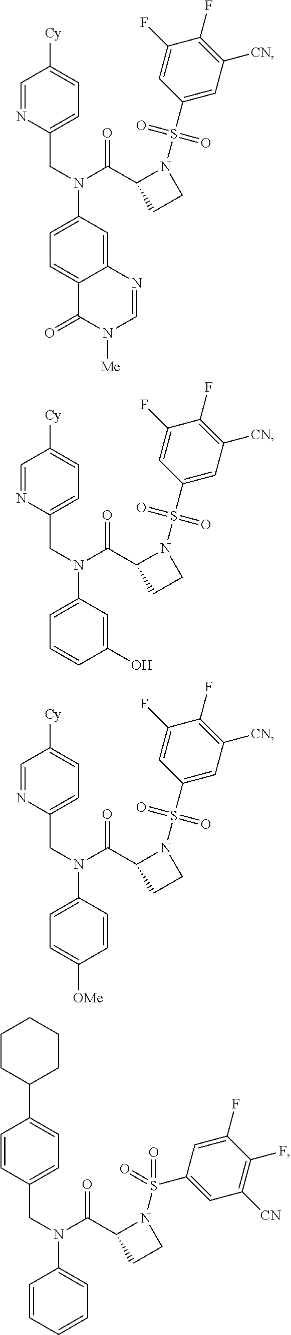 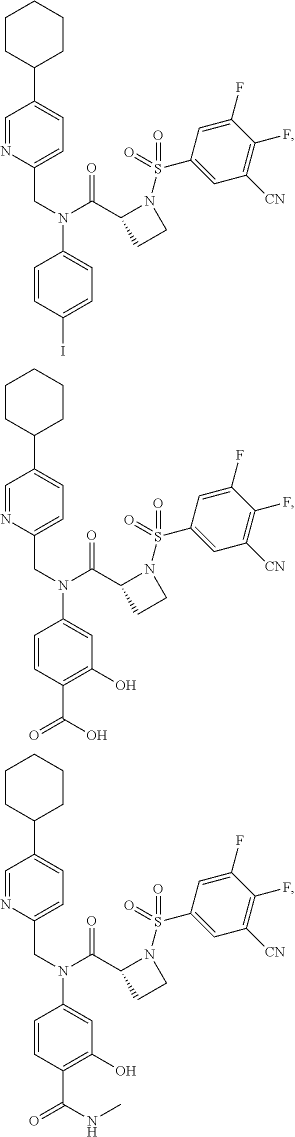 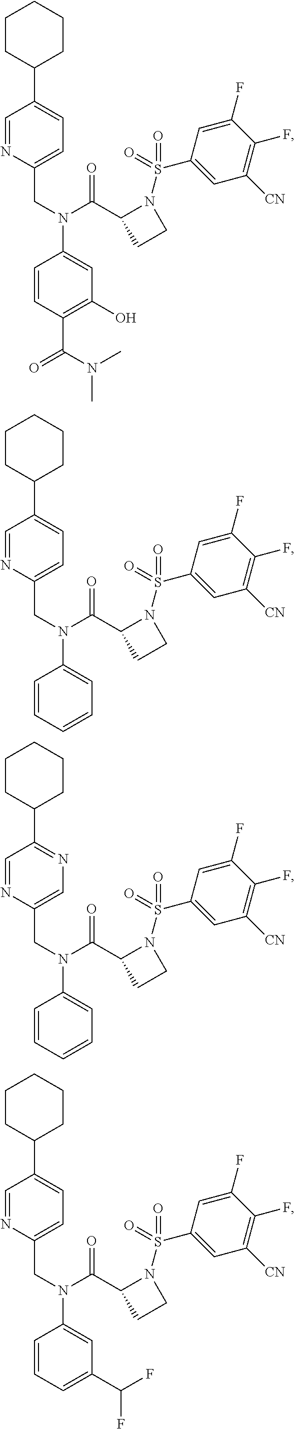 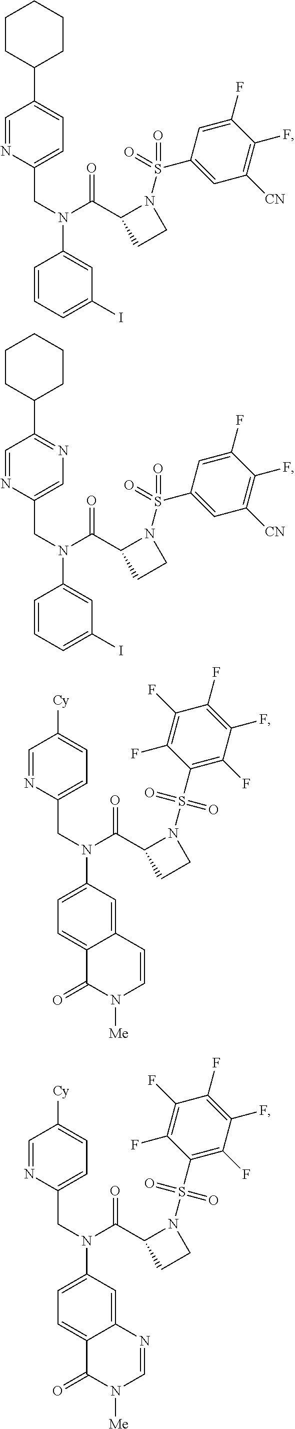 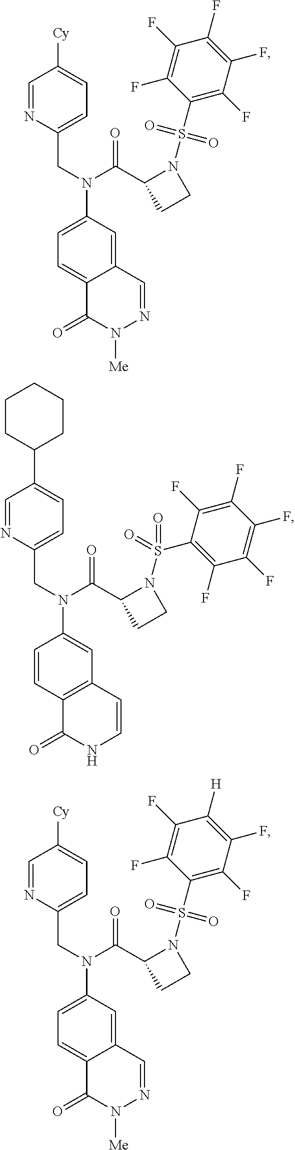 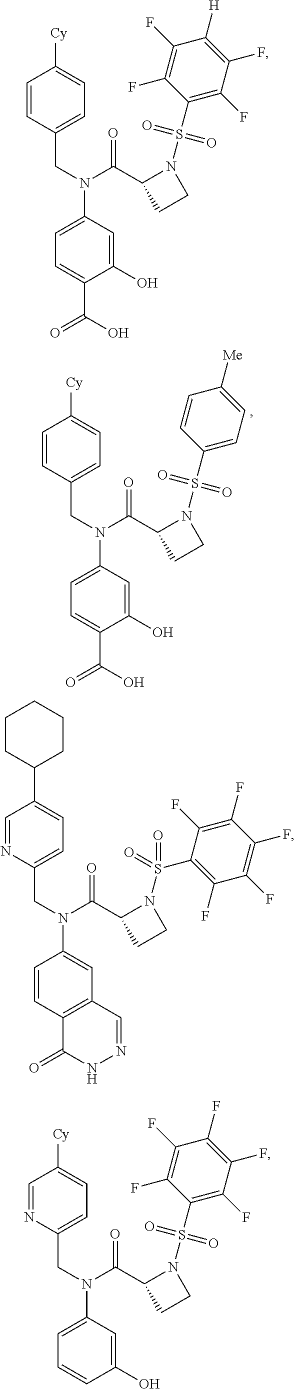 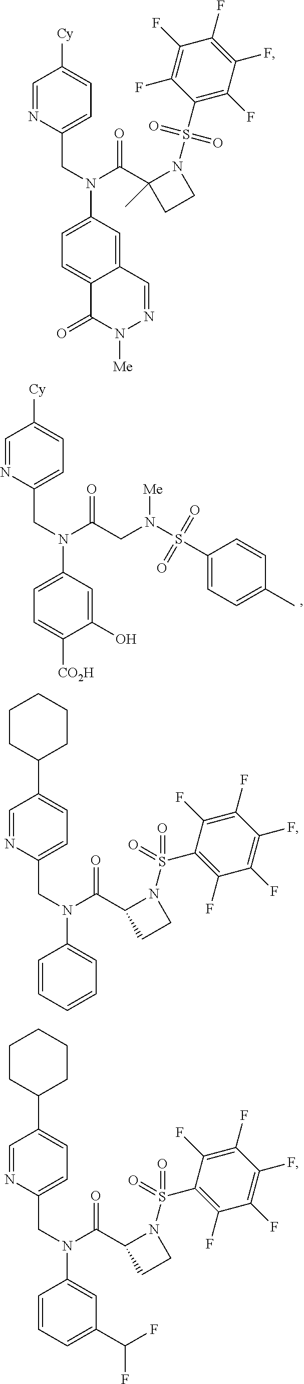 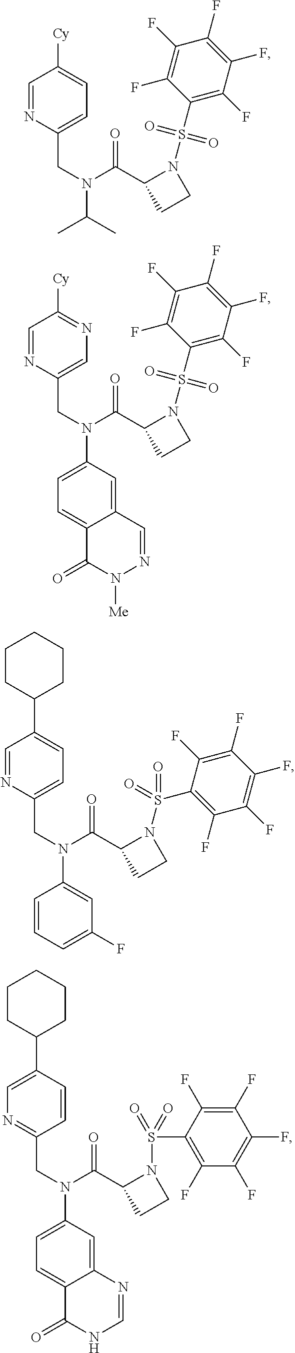
wherein Cy is cyclopropyl,
and solvates, hydrates, or pharmaceutically acceptable salts thereof.

12. A pharmaceutical composition comprising a therapeutically effective amount of a compound, solvate, hydrate, or pharmaceutically acceptable salt, of any one of claims 1-11, and a pharmaceutically acceptable excipient.

13. A composition for use in selectively treating tumor cells having a constituitively activated Stat3, comprising a therapeutically effective amount of a compound of any one of claims 1-11.

14. A compound of claim 1 having a Stat3 DNA-binding activity as measured by electrophoretic mobility shift assay (EMSA) of less than 5 micromolar, preferably of less than 1 micromolar.

15. A method of treating cancer, comprising administering to a subject in need thereof, a therapeutically effective amount of a composition of claim 12.

16. A method of administering a composition of claim 12 to a subject, wherein survival, growth or migration of a cell harboring abberantly active Stat3 is inhibited.

17. The method of claim 15, wherein the effective dose of the composition ranges from about 0.05 mg/kg to about 5 g/kg, from about 0.08 mg/kg to about 0.5 mg/kg, from about 0.08 to about 0.24 mg/kg, or from about 0.24 to about 0.5 mg/kg, or from about 0.08 to 0.5 mg/kg.

18. The method of claim 15, wherein the one or more effective doses of the composition are administered orally, subcutaneously, intravenously, or intramuscularly.

19. The method of claim 15, wherein the cancer is a solid tumor, preferably a solid tumor which is selected from glioma, breast cancer, pancreatic cancer, lung cancer, prostate cancer, ovarian cancer, bladder cancer, head and neck cancer, thyroid cancer, brain cancer, skin cancer and kidney cancer.

20. The method of claim 16, wherein the cancer is selected from the group consisting of: medulloblastomas, cerebral menangiomas, malignant melanoma, multiple myeloma, lymphomas, including anaplastic large T cell lymphoma, sezary syndrome, EBV-related Burkitt's Lymphoma, HSV Saimiri-dependent (T Cell), cutaneous T cell lymphoma, mycosis fungoides, leukemia, including HTLV-I dependent leukemia, erythroleukemia, acute lymphocytic leukemia (ALL), chronic lymphocytic leukemia (CLL), acute myelogenous leukemia (AML), chronic myelogenous leukemia (CML), megakaryocytic leukemia, and large granula lymphocyte (LGL) leukemia, renal cell carcinoma, pancreatic adenocarcinoma, ovarian carcinoma, squamous cell carcinoma of the head and neck, and Hodgkin's Lymphoma.

21. The use of a compound of any one of claims 1-11 for the preparation of a medicament for the treatment of a condition selected from the group consisting of cancer, hyperplasia, or neoplasia.
